(19) United States
(12) Patent Application Publication
(10) Pub. No.: US 2025/0066774 A1
(43) Pub. Date: Feb. 27, 2025

(54) Engineered Guide RNAs and Polynucleotides

(71) Applicant: Shape Therapeutics Inc., Seattle, WA (US)

(72) Inventors: Lan GUO, Seattle, WA (US); Richard Thomas SULLIVAN, Seattle, WA (US); Lina Rajili BAGEPALLI, Cambridge, MA (US); Yiannis SAVVA, Seattle, WA (US); Adrian Wrangham BRIGGS, Seattle, WA (US); Jason Thaddeus DEAN, Seattle, WA (US); Brian BOOTH, West Roxbury, MA (US)

(21) Appl. No.: 18/677,968

(22) Filed: May 30, 2024

Related U.S. Application Data
(63) Continuation of application No. PCT/US2022/080703, filed on Nov. 30, 2022.
(60) Provisional application No. 63/284,738, filed on Dec. 1, 2021.
(60) Provisional application No. 63/327,381, filed on Apr. 5, 2022.
(60) Provisional application No. 63/419,436, filed on Oct. 26, 2022.

(51) Int. Cl. C12N 15/11; A61P 1/00; A61P 25/16; C12N 15/86

(52) U.S. Cl. CPC C12N 15/111; A61P 1/00; A61P 25/16; C12N 15/86; C12N 2310/531; C12N 2750/14143

(57) ABSTRACT

Disclosed herein are engineered latent guide RNAs targeting LRRK2 and compositions comprising the same for treatment of diseases or conditions (e.g. Parkinson's Disease) in a subject. Also disclosed herein are methods of treating diseases or conditions (e.g. Parkinson's Disease) in a subject by administering engineered latent guide RNAs or pharmaceutical compositions described herein.

### CROSS REFERENCE

This application is a continuation of PCT/US2022/080703, filed Nov. 30, 2022, which claims priority under 35 U.S.C. § 119 from Provisional Application Ser. No. 63/284,738, filed Dec. 1, 2021, Provisional Application Ser. No. 63/327,381, filed Apr. 5, 2022, and Provisional Application Ser. No. 63/419,436 filed Oct. 26, 2022, the disclosures of which are incorporated herein by reference in their entirety.

### INCORPORATION BY REFERENCE OF SEQUENCE LISTING

The instant application contains a Sequence Listing, which has been submitted via Patent Center. The Sequence Listing titled 199235_748301_SL.xml which was created on May 24, 2024 and is 429,266 bytes in size, is hereby incorporated by reference in its entirety.

### SUMMARY

Disclosed herein is an engineered latent guide RNA wherein: (a) upon hybridization to a sequence of a target LRRK2 RNA, forms a guide-target RNA scaffold with the sequence of the target LRRK2 RNA; (b) formation of the guide-target RNA scaffold substantially forms a micro-footprint that comprises one or more structural features selected from the group consisting of: a mismatch, a bulge, an internal loop, and a hairpin; (c) the structural feature is not present within the engineered latent guide RNA prior to the hybridization of the engineered latent guide RNA to the LRRK2 target RNA; (d) upon hybridization of the engineered latent guide RNA to the sequence of the target LRRK2 RNA, the engineered latent guide RNA facilitates RNA editing of an on-target adenosine in the sequence of the target LRRK2 RNA by an RNA editing entity; and (e) the engineered latent guide RNA has at least 80%, at least 85%, at least 90%, at least 92%, at least 95%, at least 99%, or 100% sequence identity to any one of SEQ ID NO: 2-SEQ ID NO: 395 or SEQ ID NO: 398-SEQ ID NO: 427. In some embodiments, the engineered latent guide RNA comprises the polynucleotide sequence of any one of SEQ ID NO: 2-SEQ ID NO: 395 or SEQ ID NO: 398-SEQ ID NO: 427. In some embodiments, the engineered latent guide RNA comprises at least 20-50 contiguous nucleotides from a portion of any one of SEQ ID NO: 2-SEQ ID NO: 395 or SEQ ID NO: 398-SEQ ID NO: 427. In some embodiments, the sequence further comprises one or more of the following: T at position −7, T at position −6, G at position −3, A at position −2, G at position −1, C at position 1, C at position 2, T at position 3, G at position 4, and T at position 10, wherein these positions are relative to the target adenosine in the sequence of a target LRRK2 RNA targeted for editing by an RNA editing entity. In some embodiments, the engineered latent guide RNA comprises a cytosine that, when the engineered latent guide RNA is hybridized to the target RNA, is present in the guide-target RNA scaffold opposite the target adenosine that is edited by the RNA editing entity, thereby forming an A/C mismatch in the guide-target RNA scaffold. In some embodiments, the guide-target RNA scaffold comprises a barbell macro-footprint that comprises a first internal loop and a second internal loop that each flank opposing ends of the micro-footprint, wherein the first internal loop is 5′ of the micro-footprint and the second internal loop is a 3′ of the micro-footprint, and wherein the first internal loop and the second internal loop facilitate an increase in the amount of the editing of the target adenosine in the target RNA, relative to an otherwise comparable engineered guide RNA lacking the first internal loop and the second internal loop. In some embodiments, the first internal loop is positioned from about 7 bases away from the A/C mismatch to about 30 bases away from the A/C mismatch with respect to the base of the first internal loop that is most proximal to the A/C mismatch. In some embodiments, the first internal loop is positioned 10 bases away from the A/C mismatch with respect to the base of the first internal loop that is most proximal to the A/C mismatch. In some embodiments, the second internal loop is positioned from about 18 bases away from the A/C mismatch to about 34 bases away from the A/C mismatch with respect to the base of the second internal loop that is most proximal to the A/C mismatch. In some embodiments, the second internal loop is positioned 34 bases away from the A/C mismatch with respect to the base of the second internal loop that is most proximal to the A/C mismatch. In some embodiments, the target LRRK2 RNA encodes a LRRK2 polypeptide having a mutation with respect to a wild-type LRRK2 polypeptide, wherein the mutation is selected from the group consisting of: E10L, A30P, S52F, E46K, A53T, L119P, A211V, C228S, E334K, N363S, V366M, A419V, R506Q, N544E, N551K, A716V, M712V, I723V, P755L, R793M, I810V, K871E, Q923H, Q930R, R1067Q, S1096C, Q111H, I1122V, A1151T, L1165P, I1192V, H1216R, S1228T, P1262A, R1325Q, I1371V, R1398H, T1410M, D1420N, R1441G, R1441H, A1442P, P1446L, V1450I, K1468E, R1483Q, R1514Q, P1542S, V1613A, R1628P, M1646T, S1647T, Y1699C, R1728H, R1728L, L1795F, M1869V, M1869T, L1870F, E1874X, R1941H, Y2006H, I2012T, G2019S, I2020T, T2031S, N2081D, T2141M, R2143H, Y2189C, T2356I, G2385R, V2390M, E2395K, M2397T, L2466H, and Q2490NfsX3. In some embodiments, the mutation is a G2019S mutation. In some embodiments, the one or more structural features of the micro-footprint comprises a bulge, wherein the bulge is a symmetric bulge. In some embodiments, the one or more structural features of the micro-footprint comprises a bulge, wherein the bulge is an asymmetric bulge. In some embodiments, the one or more structural features of the micro-footprint comprises an internal loop, wherein the internal loop is a symmetric internal loop. In some embodiments, the one or more structural features of the micro-footprint comprises an internal loop, wherein the internal loop is an asymmetric internal loop. In some embodiments, the one or more structural features of the micro-footprint comprises a Wobble base pair. In some embodiments, the one or more structural features of the micro-footprint comprises a hairpin, wherein the hairpin is a recruitment hairpin or a non-recruitment hairpin. In some embodiments, the RNA editing entity comprises ADAR1, ADAR2, ADAR3, or any combination thereof. In some embodiments, the engineered latent guide RNA is encoded by an engineered polynucleotide. In some embodiments, the engineered polynucleotide is comprised in or on a vector. In some embodiments, the vector is a viral vector, and wherein the engineered polynucleotide is encapsidated in the viral vector. In some embodiments, the viral vector is an adeno-associated viral (AAV) vector or a derivative thereof. In some embodiments, the AAV vector is AAV1, AAV2, AAV3, AAV4, AAV5, AAV6, AAV7, AAV8, AAV9, AAV10, AAV11, or a derivative, a chimera, or a variant thereof. In some embodiments, the AAV vector is a recombinant AAV (rAAV) vector, a hybrid AAV vector, a chimeric AAV vector, a self-complementary AAV (scAAV) vector, or any combination thereof.

Also disclosed herein is an engineered latent guide RNA wherein: (a) upon hybridization to a sequence of a target LRRK2 RNA, forms a guide-target RNA scaffold with the sequence of the target LRRK2 RNA; (b) formation of the guide-target RNA scaffold substantially forms a micro-footprint that comprises one or more structural features selected from the group consisting of: a mismatch, a bulge, an internal loop, and a hairpin; (c) the structural feature is not present within the engineered latent guide RNA prior to the hybridization of the engineered latent guide RNA to the LRRK2 target RNA; (d) upon hybridization of the engineered latent guide RNA to the sequence of the target LRRK2 RNA, the engineered latent guide RNA facilitates RNA editing of an on-target adenosine in the sequence of the target LRRK2 RNA by an RNA editing entity; and (e) the sequence further comprises one or more of the following: T at position −7, T at position −6, G at position −3, A at position −2, G at position −1, C at position 1, C at position 2, T at position 3, G at position 4, and T at position 10, wherein these positions are relative to the target adenosine in the sequence of a target LRRK2 RNA targeted for editing by an RNA editing entity.

Also disclosed herein is a pharmaceutical composition comprising: (a) an engineered latent guide RNA as described herein; and (b) a pharmaceutically acceptable: excipient, carrier, or diluent.

Also disclosed herein is a method of treating a disease or a condition in a subject in need thereof, the method comprising administering to the subject a therapeutically effective amount of an engineered latent guide RNA as described herein or a pharmaceutical composition as described herein. In some embodiments, the disease or condition comprises Parkinson's disease. In some embodiments, the disease or condition comprises Crohn's disease. In some embodiments, the subject has a mutation in an LRRK2 polypeptide selected from the group consisting of: E10L, A30P, S52F, E46K, A53T, L119P, A211V, C228S, E334K, N363S, V366M, A419V, R506Q, N544E, N551K, A716V, M712V, I723V, P755L, R793M, I810V, K871E, Q923H, Q930R, R1067Q, S1096C, Q1111H, I1122V, A1151T, L1165P, I1192V, H1216R, S1228T, P1262A, R1325Q, I1371V, R1398H, T1410M, D1420N, R1441G, R1441H, A1442P, P1446L, V1450I, K1468E, R1483Q, R1514Q, P1542S, V1613A, R1628P, M1646T, S1647T, Y1699C, R1728H, R1728L, L1795F, M1869V, M1869T, L1870F, E1874X, R1941H, Y2006H, I2012T, G2019S, I2020T, T2031S, N2081D, T2141M, R2143H, Y2189C, T2356I, G2385R, V2390M, E2395K, M2397T, L2466H, and Q2490NfsX3. In some embodiments, the mutation in the LRRK2 polypeptide is associated with the disease or condition. In some embodiments, the mutation in the LRRK2 polypeptide is G2019S. In some embodiments, the subject is human or a non-human animal.

### BRIEF DESCRIPTION OF THE DRAWINGS

The novel features of the present disclosure are set forth with particularity in the appended claims. A better understanding of the features and advantages of embodiments of the present disclosure will be obtained by reference to the following detailed description that sets forth illustrative embodiments, and the accompanying drawings of which:

FIG. 1 shows a legend of various exemplary structural features present in guide-target RNA scaffolds formed upon hybridization of a latent guide RNA of the present disclosure to a target RNA. Example structural features shown include an 8/7 asymmetric loop (8 nucleotides on the target RNA side and 7 nucleotides on the guide RNA side), a 2/2 symmetric bulge (2 nucleotides on the target RNA side and 2 nucleotides on the guide RNA side), a 1/1 mismatch (1 nucleotide on the target RNA side and 1 nucleotide on the guide RNA side), a 5/5 symmetric internal loop (5 nucleotides on the target RNA side and 5 nucleotides on the guide RNA side), a 24 bp region (24 nucleotides on the target RNA side base paired to 24 nucleotides on the guide RNA side), and a 2/3 asymmetric bulge (2 nucleotides on the target RNA side and 3 nucleotides on the guide RNA side).

FIG. 2 shows a summary of how a library for screening longer self-annealing RNA structures was generated.

FIG. 3 shows a comparison of cell-free RNA editing using the high throughput described here versus in-cell RNA editing facilitated via the same engineered guide RNA sequence at various timepoints.

FIG. 4 shows heatmaps of all self-annealing RNA structures tested for 4 micro-footprints (A/C mismatch, 2108, 871, and 919) formed within varying placement of a barbell macro-footprint. The y-axis shows all candidate engineered guide RNAs tested and the x-axis shows the target sequence positions, with position 0 representing the target adenosine to be edited.

FIGS. 5A-D show LRRK2 RNA editing profiles of various engineered guide RNAs of the present disclosure

FIG. 6 shows the LRRK2 RNA editing profile of an engineered guide RNA of the present disclosure, which forms a barbell macro-footprint and a micro-footprint in the guide-target RNA scaffold.

FIGS. 7A-7C depict the ADAR-mediated RNA editing efficiency of guide RNAs designed through machine learning targeting LRRK2 in an in-cell editing model, each having a barbell macro-footprint with symmetrical internal loops at positions −20 and +26.

FIG. 8 shows LRRK2 target RNA editing for a control engineered guide and exemplary engineered guide 919 via ADAR1 and ADAR1+ADAR2.

FIG. 9 shows LRRK2 target RNA editing for exemplary engineered guide 1976 and exemplary engineered guide 2397 via ADAR1 and ADAR1+ADAR2.

FIG. 10 shows LRRK2 target RNA editing for exemplary engineered guide 871 and exemplary engineered guide 610 via ADAR1 and ADAR1+ADAR2.

FIG. 11 shows LRRK2 target RNA editing for exemplary engineered guides ML generative 0703 and ML generative 0719 designed by machine learning via ADAR1 and ADAR1+ADAR2.

FIG. 12 shows LRRK2 target RNA editing for exemplary engineered guides ML generative 0728 and ML generative 0732 designed by machine learning via ADAR1 and ADAR1+ADAR2.

FIG. 13 shows LRRK2 target RNA editing for exemplary engineered guides ML generative 0733 and ML generative 0742 designed by machine learning via ADAR1 and ADAR1+ADAR2.

FIG. 14 shows LRRK2 target RNA editing for exemplary engineered guides ML generative 0743 and ML generative 0745 designed by machine learning via ADAR1 and ADAR1+ADAR2.

FIG. 15 shows LRRK2 target RNA editing for exemplary engineered guides ML generative 0766 and ML generative 0769 designed by machine learning via ADAR1 and ADAR1+ADAR2.

FIG. 16 shows LRRK2 target RNA editing for exemplary engineered guides ML generative 0766 and ML generative 0769 designed by machine learning via ADAR1 and ADAR1+ADAR2.

FIG. 17 shows LRRK2 target RNA editing for exemplary engineered guides ML exhaustive 0049 and ML exhaustive 0069 designed by machine learning via ADAR1 and ADAR1+ADAR2.

FIG. 18 shows LRRK2 target RNA editing for exemplary engineered guides ML exhaustive 0090 and ML exhaustive 0139 designed by machine learning via ADAR1 and ADAR1+ADAR2.

FIG. 19 shows LRRK2 target RNA editing for exemplary engineered guides ML generative 0274 and ML generative 0325 designed by machine learning via ADAR1 and ADAR1+ADAR2.

FIG. 20 shows LRRK2 target RNA editing for exemplary engineered guides ML generative 0332 and ML generative 0559 designed by machine learning via ADAR1 and ADAR1+ADAR2.

FIG. 21 shows LRRK2 target RNA editing for exemplary engineered guides ML generative 0639 and ML generative 0643 designed by machine learning via ADAR1 and ADAR1+ADAR2.

FIG. 22 shows LRRK2 target RNA editing for exemplary engineered guides ML generative 0644 and ML generative 0690 designed by machine learning via ADAR1 and ADAR1+ADAR2.

FIG. 23 shows LRRK2 target RNA editing for exemplary engineered guides ML generative 0699 and ML generative 0701 designed by machine learning via ADAR1 and ADAR1+ADAR2.

FIG. 24 shows LRRK2 target RNA editing for exemplary engineered guides ML exhaustive 0395 and ML exhaustive 0453 designed by machine learning via ADAR1 and ADAR1+ADAR2.

FIG. 25 shows LRRK2 target RNA editing for exemplary engineered guides ML exhaustive 0464 and ML exhaustive 1042 designed by machine learning via ADAR1 and ADAR1+ADAR2.

FIG. 26 shows LRRK2 target RNA editing for exemplary engineered guides ML generative 0002 and ML generative 0013 designed by machine learning via ADAR1 and ADAR1+ADAR2.

FIG. 27 shows LRRK2 target RNA editing for exemplary engineered guides ML generative 0016 and ML generative 0043 designed by machine learning via ADAR1 and ADAR1+ADAR2.

FIG. 28 shows LRRK2 target RNA editing for exemplary engineered guides ML generative 0058 and ML generative 0071 designed by machine learning via ADAR1 and ADAR1+ADAR2.

FIG. 29 shows LRRK2 target RNA editing for exemplary engineered guides ML generative 0130 and ML generative 0156 designed by machine learning via ADAR1 and ADAR1+ADAR2.

FIG. 30 shows LRRK2 target RNA editing for exemplary engineered guides ML generative 0176 and ML generative 0218 designed by machine learning via ADAR1 and ADAR1+ADAR2.

FIG. 31 shows LRRK2 target RNA editing for exemplary engineered guides ML exhaustive 1045 and ML exhaustive 1540 designed by machine learning via ADAR1 and ADAR1+ADAR2.

FIG. 32 shows LRRK2 target RNA editing for exemplary engineered guides ML exhaustive 0315 and ML exhaustive 0414 designed by machine learning via ADAR1 and ADAR1+ADAR2.

FIG. 33 shows LRRK2 target RNA editing for exemplary engineered guide ML exhaustive 0013 designed by machine learning via ADAR1 and ADAR1+ADAR2.

FIG. 34A-34B depict selection of two exemplary LRRK2 guide RNAs designed through machine learning for further engineering.

FIG. 35 shows a plot of editing specificity of LRRK2 exhaustive guide RNAs designed through machine learning via ADAR1, ADAR2, or ADAR1+ADAR2.

FIG. 36 shows exemplary LRRK2 exhaustive guide RNAs designed through machine learning that display specificity for ADAR2.

FIGS. 37A and 37B show the top performing guide RNAs that display specificity for ADAR1+ADAR2.

FIGS. 38A and 38B show the top performing guide RNAs that display specificity for ADAR2.

FIGS. 39A and 39B show the top performing guide RNAs that display specificity for ADAR1.

FIG. 40 depicts a comparison between ML-derived gRNAs and gRNAs generated using in vitro high throughput screening (HTS) methods.

FIG. 41 depicts an overview of the engineering of guide RNAs produced from high-throughput screening.

FIGS. 42A and 42B depict cell-free and in-cell editing of exemplary LRRK2 guide610 without a barbell macro-footprint (FIG. 42A) and with a barbell macro-footprint (FIG. 42B) via ADAR.

FIGS. 43A-43C show engineering of the macro-footprint position for an exemplary guide610 targeting LRRK2. FIG. 43A shows tiling of the macro-footprint positioning for the exemplary guide with respect to the A/C mismatch, and how this tiling affects editing via ADAR1 and ADAR1+ADAR2. FIG. 43B shows the percent editing for the guide variants via ADAR1. FIG. 43C shows the percent editing for the guide variants via ADAR1+ADAR2.

FIGS. 44A-44C show engineering of right barbell coordinates for an exemplary guide610 targeting LRRK2. As shown in FIG. 44A, the coordinate of the right barbell was tiled between the following coordinates with respect to the A/C mismatch: +22. +23, +24, +25, +26, +28, +30, +32, and +34, and the effect of each position on ADAR1 and ADAR1+ADAR2 editing was determined. FIG. 44B shows the percent editing for the exemplary guide variants via ADAR1. FIG. 44C shows the percent editing for the exemplary guide variants via ADAR1+ADAR2.

FIGS. 45A and 45B show engineering of left barbell coordinates for an exemplary guide targeting LRRK2. As shown in FIG. 45A, the coordinate of the left barbell was tiled between the following coordinates with respect to the A/C mismatch: −10, −12, −14, −16, −18, −20, −22, and −24, and the effect of each position on ADAR1 and ADAR1+ADAR2 editing was determined. FIG. 45B shows the percent editing for the exemplary guide variants via ADAR1.

FIGS. 46A and 46B show engineering of guide length for an exemplary guide targeting LRRK2. FIG. 46A depicts the effect of guide length on ADAR1 and ADAR1+ADAR2 editing. FIG. 46B shows the percent editing for the exemplary guide variants of varying length via ADAR1. The y-axis shows all candidate engineered guide RNAs tested and the x-axis shows the target sequence positions, with position 0 representing the target adenosine to be edited.

FIGS. 47A and 47B show in cell and cell-free editing of LRRK2 by exemplary guide RNA 2063 variants without a barbell (FIG. 47A) and having a barbell (FIG. 47B) via ADAR1 and ADAR1+ADAR2. The y-axis shows all candidate engineered guide RNAs tested and the x-axis shows the target sequence positions, with position 0 representing the target adenosine to be edited.

FIGS. 48A and 48B show in cell and cell-free editing of LRRK2 by exemplary guide RNA 1590 variants without a barbell (FIG. 48A) and having a barbell (FIG. 48B) via ADAR1 and ADAR1+ADAR2. The y-axis shows all candidate engineered guide RNAs tested and the x-axis shows the target sequence positions, with position 0 representing the target adenosine to be edited.

FIGS. 49A-49C show in cell and cell-free editing of LRRK2 by exemplary guide RNA 2397 variants without a barbell (FIG. 49A) and having a barbell (FIG. 49B and FIG. 49C) via ADAR1 and ADAR1+ADAR2. The y-axis shows all candidate engineered guide RNAs tested and the x-axis shows the target sequence positions, with position 0 representing the target adenosine to be edited.

FIGS. 50A-50C show engineering of the macro-footprint positioning for exemplary guide 2397 RNA variants. FIG. 50A depicts a summary of the RNA editing efficiencies for the exemplary guide 2397 RNA variants, while FIG. 50B and FIG. 50C depict the editing efficiency by position for each exemplary guide RNA via ADAR1 (FIG. 50B) and ADAR1+ADAR2 (FIG. 50C). The y-axis shows all candidate engineered guide RNAs tested and the x-axis shows the target sequence positions, with position 0 representing the target adenosine to be edited.

FIGS. 51A-51C show engineering of the right barbell coordinate for exemplary guide 2397 RNA variants. FIG. 51A depicts a summary of the RNA editing efficiencies for the exemplary guide 2397 RNA variants, while FIG. 51B and FIG. 51C depict the editing efficiency by position for each exemplary guide RNA via ADAR1 (FIG. 51B) and ADAR1+ADAR2 (FIG. 51C). The y-axis shows all candidate engineered guide RNAs tested and the x-axis shows the target sequence positions, with position 0 representing the target adenosine to be edited.

FIG. 52 depicts engineering of the left barbell coordinate for exemplary guide 2397 RNA variants.

FIGS. 53A and 53B show in cell and cell-free editing of LRRK2 by exemplary guide RNA 1321 variants without a barbell (FIG. 53A) and having a barbell (FIG. 53B) via ADAR1 and ADAR1+ADAR2. The y-axis shows all candidate engineered guide RNAs tested and the x-axis shows the target sequence positions, with position 0 representing the target adenosine to be edited.

FIGS. 54A and 54B show in cell and cell-free editing of LRRK2 by exemplary guide RNA 295 variants without a barbell (FIG. 54A) and having a barbell (FIG. 54B) via ADAR1 and ADAR1+ADAR2. The y-axis shows all candidate engineered guide RNAs tested and the x-axis shows the target sequence positions, with position 0 representing the target adenosine to be edited.

FIGS. 55A and 55B show in cell and cell-free editing of LRRK2 by exemplary guide RNA 730 variants without a barbell (FIG. 55A) and having a barbell (FIG. 55B) via ADAR1 and ADAR1+ADAR2. The y-axis shows all candidate engineered guide RNAs tested and the x-axis shows the target sequence positions, with position 0 representing the target adenosine to be edited.

FIGS. 56A and 56B show in cell and cell-free editing of LRRK2 by exemplary guide RNA 708 variants without a barbell (FIG. 56A) and having a barbell (FIG. 56B) via ADAR1 and ADAR1+ADAR2. The y-axis shows all candidate engineered guide RNAs tested and the x-axis shows the target sequence positions, with position 0 representing the target adenosine to be edited.

FIGS. 57A and 57B show in cell and cell-free editing of LRRK2 by exemplary guide RNA 351 variants without a barbell (FIG. 57A) and having a barbell (FIG. 57B) via ADAR1 and ADAR1+ADAR2. The y-axis shows all candidate engineered guide RNAs tested and the x-axis shows the target sequence positions, with position 0 representing the target adenosine to be edited.

FIGS. 58A and 58B show in cell and cell-free editing of LRRK2 by exemplary guide RNA 1326 variants without a barbell (FIG. 58A) and having a barbell (FIG. 58B) via ADAR1 and ADAR1+ADAR2. The y-axis shows all candidate engineered guide RNAs tested and the x-axis shows the target sequence positions, with position 0 representing the target adenosine to be edited.

FIGS. 59A-590 show in cell and cell-free editing of LRRK2 by exemplary guide RNA 871 variants without a barbell (FIG. 59A) and having barbells (FIG. 59B-FIG. 590) via ADAR1 and ADAR1+ADAR2. The y-axis shows all candidate engineered guide RNAs tested and the x-axis shows the target sequence positions, with position 0 representing the target adenosine to be edited.

FIGS. 60A-60C show engineering of the macro-footprint positioning for exemplary guide 871 RNA variants. FIG. 60A depicts a summary of the RNA editing efficiencies for the exemplary guide 871 RNA variants, while FIG. 60B and FIG. 60C depict the editing efficiency by position for each exemplary guide RNA via ADAR1 (FIG. 60B) and ADAR1+ADAR2 (FIG. 60C). The y-axis shows all candidate engineered guide RNAs tested and the x-axis shows the target sequence positions, with position 0 representing the target adenosine to be edited.

FIGS. 61A-61C show engineering of the right barbell coordinate for exemplary guide 871 RNA variants. FIG. 61A depicts a summary of the RNA editing efficiencies for the exemplary guide 871 RNA variants, while FIG. 61B and FIG. 61C depict the editing efficiency by position for each exemplary guide RNA via ADAR1 (FIG. 61B) and ADAR1+ADAR2 (FIG. 61C). The y-axis shows all candidate engineered guide RNAs tested and the x-axis shows the target sequence positions, with position 0 representing the target adenosine to be edited.

FIGS. 62A-62C show engineering of the left barbell coordinate for exemplary guide 871 RNA variants. FIG. 62A depicts a summary of the RNA editing efficiencies for the exemplary guide 871 RNA variants, while FIG. 62B and FIG. 62C depict the editing efficiency by position for each exemplary guide RNA via ADAR1 (FIG. 62B) and ADAR1+ADAR2 (FIG. 62C). The y-axis shows all candidate engineered guide RNAs tested and the x-axis shows the target sequence positions, with position 0 representing the target adenosine to be edited.

FIGS. 63A-63C show engineering of the guide length for exemplary guide 871 RNA variants. FIG. 63A depicts a summary of the RNA editing efficiencies for the exemplary guide 871 RNA variants, while FIG. 63B and FIG. 63C depict the editing efficiency by position for each exemplary guide RNA via ADAR1 (FIG. 63B) and ADAR1+ADAR2 (FIG. 63C). The y-axis shows all candidate engineered guide RNAs tested and the x-axis shows the target sequence positions, with position 0 representing the target adenosine to be edited.

FIGS. 64A-64T show in cell and cell-free editing of LRRK2 by exemplary guide RNA 919 variants. FIG. 64A provides a summary of the in cell editing data for the exemplary guide 919 variants via ADAR1 and ADAR1+ADAR2. FIG. 64B-FIG. 64T depict the editing efficiency by position for each exemplary guide 919 RNA via ADAR1 and ADAR1+ADAR2. The y-axis shows all candidate engineered guide RNAs tested and the x-axis shows the target sequence positions, with position 0 representing the target adenosine to be edited.

FIGS. 65A-65C show engineering of the macro-footprint positioning for exemplary guide 919 RNA variants. FIG. 65A depicts a summary of the RNA editing efficiencies for the exemplary guide 919 RNA variants, while FIG. 65B and FIG. 65C depict the editing efficiency by position for each exemplary guide RNA via ADAR1 (FIG. 65B) and ADAR1+ADAR2 (FIG. 65C). The y-axis shows all candidate engineered guide RNAs tested and the x-axis shows the target sequence positions, with position 0 representing the target adenosine to be edited.

FIGS. 66A-66C show engineering of the right barbell coordinate for exemplary guide 919 RNA variants. FIG. 66A depicts a summary of the RNA editing efficiencies for the exemplary guide 919 RNA variants, while FIG. 66B and FIG. 66C depict the editing efficiency by position for each exemplary guide RNA via ADAR1 (FIG. 66B) and ADAR1+ADAR2 (FIG. 66C). The y-axis shows all candidate engineered guide RNAs tested and the x-axis shows the target sequence positions, with position 0 representing the target adenosine to be edited.

FIGS. 67A-67C show engineering of the left barbell coordinate for exemplary guide 919 RNA variants. FIG. 67A depicts a summary of the RNA editing efficiencies for the exemplary guide 919 RNA variants, while FIG. 67B and FIG. 67C depict the editing efficiency by position for each exemplary guide RNA via ADAR1 (FIG. 67B) and ADAR1+ADAR2 (FIG. 67C). The y-axis shows all candidate engineered guide RNAs tested and the x-axis shows the target sequence positions, with position 0 representing the target adenosine to be edited.

FIGS. 68A-68C show engineering of the guide length for exemplary guide 919 RNA variants. FIG. 68A depicts a summary of the RNA editing efficiencies for the exemplary guide 919 RNA variants, while FIG. 68B and FIG. 68C depict the editing efficiency by position for each exemplary guide RNA via ADAR1 (FIG. 68B) and ADAR1+ADAR2 (FIG. 68C). The y-axis shows all candidate engineered guide RNAs tested and the x-axis shows the target sequence positions, with position 0 representing the target adenosine to be edited.

FIGS. 69A-69C show in cell and cell-free editing of LRRK2 by exemplary guide RNA 844 variants via ADAR1 and ADAR1+ADAR2. The y-axis shows all candidate engineered guide RNAs tested and the x-axis shows the target sequence positions, with position 0 representing the target adenosine to be edited.

FIGS. 70A-70C show in cell and cell-free editing of LRRK2 by exemplary guide RNA 1976 variants via ADAR1 and ADAR1+ADAR2. The y-axis shows all candidate engineered guide RNAs tested and the x-axis shows the target sequence positions, with position 0 representing the target adenosine to be edited.

FIGS. 71A-71C show engineering of the macro-footprint positioning for exemplary guide 1976 RNA variants. FIG. 71A depicts a summary of the RNA editing efficiencies for the exemplary guide 1976 RNA variants, while FIG. 71B and FIG. 71C depict the editing efficiency by position for each exemplary guide RNA via ADAR1 (FIG. 71B) and ADAR1+ADAR2 (FIG. 71C). The y-axis shows all candidate engineered guide RNAs tested and the x-axis shows the target sequence positions, with position 0 representing the target adenosine to be edited.

FIGS. 72A-72C show engineering of the right barbell coordinate for exemplary guide 1976 RNA variants. FIG. 72A depicts a summary of the RNA editing efficiencies for the exemplary guide 1976 RNA variants, while FIG. 72B and FIG. 72C depict the editing efficiency by position for each exemplary guide RNA via ADAR1 (FIG. 72B) and ADAR1+ADAR2 (FIG. 72C). The y-axis shows all candidate engineered guide RNAs tested and the x-axis shows the target sequence positions, with position 0 representing the target adenosine to be edited.

FIG. 73 depicts engineering of the left barbell coordinate for exemplary guide 1976 RNA variants.

FIG. 74 shows in cell and cell-free editing of LRRK2 by an exemplary guide RNA 1700. The y-axis shows all candidate engineered guide RNAs tested and the x-axis shows the target sequence positions, with position 0 representing the target adenosine to be edited.

FIGS. 75A-75E show in cell and cell-free editing of LRRK2 by exemplary guide RNA 860 variants. The y-axis shows all candidate engineered guide RNAs tested and the x-axis shows the target sequence positions, with position 0 representing the target adenosine to be edited.

FIG. 76 shows in cell and cell-free editing of LRRK2 by an exemplary guide RNA 2108. The y-axis shows all candidate engineered guide RNAs tested and the x-axis shows the target sequence positions, with position 0 representing the target adenosine to be edited.

FIG. 77 depicts a comparison of editing efficiency between exemplary guide RNA variants targeting LRRK2.

FIG. 78 depicts an scAAV vector map for in vitro screening of LRRK2 guide RNA variant produced herein when expressed in an AAV vector.

FIGS. 79A and 79B depict editing efficiencies of exemplary LRRK2 guide provided herein when transfected as an scAAV vector plasmid (FIG. 79A) or transduced as an scAAVDJ virus (FIG. 79B) via ADAR.

FIG. 80 depicts a workflow for screening exemplary guide RNAs targeting LRRK2 in a broken GFP reporter system.

FIG. 81 depicts the editing efficiency of the exemplary guides targeting LRRK2 in the broken GFP reporter system via exogenous or endogenous ADAR

FIG. 82 provides a comparison between linear and circularized versions of exemplary guide RNAs targeting LRRK2.

FIG. 83A-FIG. 83B depict engineering of the length of circularized LRRK2 guide RNAs by increasing the length of the circularized guide RNA by an additional 15 nucleotides (FIG. 83A), 30 nucleotides (FIG. 83A), and 100 nucleotides (FIG. 83B).

FIG. 84 depicts the effect of deletion of selected uridines from an engineered circularized guide RNA targeting LRRK2 on editing of a target LRRK2 RNA.

FIG. 85A-FIG. 85D illustrate the in vivo editing of a target LRRK2 RNA upon administration of an scAAV vector encoding an engineered guide RNA targeting LRRK2. FIG. 85A and FIG. 85C depict the in vivo editing efficiencies for the scAAV vector encoding the engineered guide RNA targeting LRRK2, as measured in the brain (FIG. 85A) and liver (FIG. 85C). FIG. 85B and FIG. 85D illustrate quantitation of engineered guide RNA expression, as compared to expression of the GAPDH control, in the brain (FIG. 85B) and liver (FIG. 85D).

### DETAILED DESCRIPTION

### RNA Editing

RNA editing can refer to a process by which RNA can be enzymatically modified post synthesis at specific nucleosides. RNA editing can comprise any one of an insertion, deletion, or substitution of a nucleotide(s). Examples of RNA editing include chemical modifications, such as pseudouridylation (the isomerization of uridine residues) and deamination (removal of an amine group from cytidine to give rise to uridine, or C-to-U editing or from adenosine to inosine, or A-to-I editing). RNA editing can be used to introduce mutations, correct missense mutations, or edit coding or non-coding regions of RNA to inhibit RNA translation and effect protein knockdown.

Described herein are engineered guide RNAs that facilitate RNA editing of a LRRK2 RNA by an RNA editing entity (e.g., an adenosine Deaminase Acting on RNA (ADAR)) or biologically active fragments thereof. An engineered guide RNA as described herein can include an engineered guide RNA having a polynucleotide sequence of any one of SEQ ID NO: 2-SEQ ID NO: 395 or SEQ ID NO: 398-SEQ ID NO: 472. In some instances, ADARs can be enzymes that catalyze the chemical conversion of adenosines to inosines in RNA. Because the properties of inosine mimic those of guanosine (inosine will form two hydrogen bonds with cytosine, for example), inosine can be recognized as guanosine by the translational cellular machinery. “Adenosine-to-inosine (A-to-I) RNA editing”, therefore, effectively changes the primary sequence of RNA targets.

### Engineered Guide RNAs

Disclosed herein are engineered guide RNAs (e.g., a guide RNA having a polynucleotide sequence of any one of SEQ ID NO: 2-SEQ ID NO: 395 or SEQ ID NO: 398-SEQ ID NO: 472) and engineered polynucleotides encoding the same for site-specific, selective editing of a LRRK2 target RNA via an RNA editing entity or a biologically active fragment thereof. In some embodiments, engineered guide RNAs of the present disclosure that target LRRK2 comprise a micro-footprint sequence and/or a macro-footprint sequence that each comprise latent structures, such that when the engineered guide RNA is hybridized to the target RNA, the latent structures manifest. A latent structure, when manifested, produces at least one structural feature selected from the group consisting of: a bulge, an internal loop, a mismatch, a hairpin, and any combination thereof. In some embodiments, the engineered guide RNA of the disclosure, upon hybridization of the engineered guide RNA and the sequence of the target RNA form a guide-target RNA scaffold, comprising (i) a region that comprises at least one structural feature; and (ii) a macro-footprint, such as a first internal loop (also referred to as a “left bell” or “LB”) and a second internal loop (also referred to as a “right bell” or “RB”) that flank opposing ends of the region of the guide-target RNA scaffold, where the engineered guide RNA facilitates an increase in the amount of the targeted edit of the adenosine of the target RNA via the adenosine deaminase enzyme RNA editing entity, relative to an otherwise comparable engineered guide RNA lacking the first internal loop and the second internal loop. As described herein, a first internal loop and a second internal loop can be described with respect to their position relative to an A/C mismatch in the target RNA scaffold, where the A in the A/C mismatch is the target adenosine of the LRRK2 target RNA. In some embodiments, the first internal loop is positioned from about 7 bases away from the A/C mismatch to about 30 bases away from the A/C mismatch with respect to the base of the first internal loop that is most proximal to the A/C mismatch. In some embodiments, the first internal loop is positioned 10 bases away from the A/C mismatch with respect to the base of the first internal loop that is most proximal to the A/C mismatch. In some embodiments, the second internal loop is positioned from about 18 bases away from the A/C mismatch to about 34 bases away from the A/C mismatch with respect to the base of the second internal loop that is most proximal to the A/C mismatch. In some embodiments, the second internal loop is positioned 34 bases away from the A/C mismatch with respect to the base of the second internal loop that is most proximal to the A/C mismatch.

As described herein, a “micro-footprint” sequence refers to a sequence with latent structures that, when manifested, facilitate editing of the adenosine of a target RNA via an adenosine deaminase enzyme. A macro-footprint can serve to guide an RNA editing entity (e.g., ADAR) and direct its activity towards a micro-footprint. In some embodiments, included within the micro-footprint sequence is a nucleotide that is positioned such that, when the guide RNA is hybridized to the target RNA, the nucleotide opposes the adenosine to be edited by the adenosine deaminase and does not base pair with the adenosine to be edited. This nucleotide is referred to herein as the “mismatched position” or “mismatch” and can be a cytosine. Micro-footprint sequences as described herein have upon hybridization of the engineered guide RNA and target RNA, at least one structural feature selected from the group consisting of: a bulge, an internal loop, a mismatch, a hairpin, and any combination thereof. Engineered guide RNAs with superior micro-footprint sequences can be selected based on their ability to facilitate editing of a specific target RNA (such as LRRK2 mRNA).

In some embodiments, guide RNAs of the present disclosure (e.g., a guide RNA having a polynucleotide sequence of any one of SEQ ID NO: 2-SEQ ID NO: 395 or SEQ ID NO: 398-SEQ ID NO: 472) can further comprise a macro-footprint. In some embodiments, the macro-footprint comprises a barbell macro-footprint. A micro-footprint can serve to guide an RNA editing enzyme and direct its activity towards the target adenosine to be edited. A “barbell” as described herein refers to a pair of internal loop latent structures that manifest upon hybridization of the guide RNA to the target RNA. In some embodiments, each internal loop is positioned towards the 5′ end or the 3′ end of the guide-target RNA scaffold formed upon hybridization of the guide RNA and the target RNA. In some embodiments, each internal loop flanks opposing sides of the micro-footprint sequence. Insertion of a barbell macro-footprint sequence flanking opposing sides of the micro-footprint sequence, upon hybridization of the guide RNA to the LRRK2 target RNA, results in formation of barbell internal loops on opposing sides of the micro-footprint, which in turn comprises at least one structural feature that facilitates editing of the LRRK2 target RNA.

Provided herein are engineered guide RNAs (such as latent guide RNA that comprise a micro-footprint sequence and/or a macro-footprint sequence) and polynucleotides encoding the same; as well as compositions comprising said engineered guide RNAs or said polynucleotides. As used herein, the term “engineered” in reference to a guide RNA or polynucleotide encoding the same refers to a non-naturally occurring guide RNA or polynucleotide encoding the same. For example, the present disclosure provides for engineered polynucleotides encoding for engineered guide RNAs. In some embodiments, the engineered guide comprises RNA. In some embodiments, the engineered guide comprises DNA. In some examples, the engineered guide comprises modified RNA bases or unmodified RNA bases. In some embodiments, the engineered guide comprises modified DNA bases or unmodified DNA bases. In some examples, the engineered guide comprises both DNA and RNA bases.

In some examples, the engineered guides provided herein (e.g., a guide RNA having a polynucleotide sequence of any one of SEQ ID NO: 2-SEQ ID NO: 395 or SEQ ID NO: 398-SEQ ID NO: 472) comprise an engineered guide that can be configured, upon hybridization to a target RNA molecule, to form, at least in part, a guide-target RNA scaffold with at least a portion of a LRRK2 target RNA molecule, wherein the guide-target RNA scaffold comprises at least one structural feature, and wherein the guide-target RNA scaffold recruits an RNA editing entity and facilitates a chemical modification of a base of a nucleotide in the LRRK2 target RNA molecule by the RNA editing entity.

In some examples, a LRRK2 target RNA of an engineered guide RNA of the present disclosure can be a pre-mRNA or mRNA. In some embodiments, the engineered guide RNA of the present disclosure hybridizes to a sequence of the LRRK2 target RNA. In some embodiments, part of the engineered guide RNA (e.g., a targeting domain) hybridizes to the sequence of the LRRK2 target RNA. The part of the engineered guide RNA that hybridizes to the target RNA is of sufficient complementary to the sequence of the target RNA for hybridization to occur.

### A. Targeting Domain

Engineered guide RNAs useful for facilitating editing of a LRRK2 target RNA as disclosed herein (e.g., a guide RNA having a polynucleotide sequence of any one of SEQ ID NO: 2-SEQ ID NO: 395 or SEQ ID NO: 398-SEQ ID NO: 472) can be engineered in any way suitable for RNA editing. In some examples, an engineered guide RNA generally comprises at least a targeting sequence that allows it to hybridize to a region of a target RNA molecule. A targeting sequence can also be referred to as a “targeting domain” or a “targeting region”.

In some cases, a targeting domain of an engineered guide allows the engineered guide to target an RNA sequence through base pairing, such as Watson Crick base pairing. In some examples, the targeting sequence can be located at either the N-terminus or C-terminus of the engineered guide. In some cases, the targeting sequence can be located at both termini. The targeting sequence can be of any length. In some cases, the targeting sequence can be at least about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or up to about 200 nucleotides in length. In some cases, the targeting sequence can be no greater than about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or 200 nucleotides in length. In some examples, an engineered guide comprises a targeting sequence that can be from about 60 to about 500, from about 60 to about 200, from about 75 to about 100, from about 80 to about 200, from about 90 to about 120, or from about 95 to about 115 nucleotides in length. In some examples, an engineered guide RNA comprises a targeting sequence that can be about 100 nucleotides in length.

In some cases, a targeting domain comprises 95%, 96%, 97%, 98%, 99%, or 100% sequence complementarity to a target RNA. In some cases, a targeting sequence comprises less than 100% complementarity to a target RNA sequence. For example, a targeting sequence and a region of a target RNA that can be bound by the targeting sequence can have a single base mismatch.

### B. Engineered Guide RNAs Having a Recruiting Domain

In some examples, an engineered guide RNA useful for facilitating editing of a LRRK2 target RNA as described herein comprises a recruiting domain that recruits an RNA editing entity (e.g., ADAR), where in some instances, the recruiting domain is formed and present in the absence of binding to the LRRK2 target RNA. A “recruiting domain” can be referred to herein as a “recruiting sequence” or a “recruiting region”. In some examples, an engineered guide RNA can be configured to facilitate editing of a base of a nucleotide of a polynucleotide of a region of a LRRK2 target RNA, modulation expression of a polypeptide encoded by the LRRK2 target RNA, or both. In some cases, an engineered guide RNA can be configured to facilitate an editing of a base of a nucleotide or polynucleotide of a region of an RNA by an RNA editing entity. In order to facilitate editing, an engineered guide RNA of the disclosure can be configured to recruit an RNA editing entity. Some embodiments provide for an RNA editing entity comprising an ADAR protein, where the ADAR protein can be selected from the group consisting of an ADAR1 (e.g., human or mouse), an ADAR2 (e.g., human or mouse), and any combination thereof. Various RNA editing entity recruiting domains can be utilized. In some examples, a recruiting domain comprises: Glutamate ionotropic receptor AMPA type subunit 2 (GluR2) or Alu. In some embodiments of the disclosure, the RNA editing entity can have an ADAR protein. An ADAR protein can be selected from the group consisting of: an ADAR1, an ADAR2, and a combination of ADAR1 and ADAR2. Other embodiments can be directed to an RNA editing entity selected from the group consisting of: a human ADAR1, a mouse ADAR1, a human ADAR2, a mouse ADAR2, and any combination thereof.

In some examples, more than one recruiting domain can be included in an engineered guide RNA of the disclosure. In examples where a recruiting domain can be present, the recruiting domain can be utilized to position the RNA editing entity to effectively react with a target RNA after the targeting sequence, for example an antisense sequence, hybridizes to a target RNA. In some cases, a recruiting domain can allow for transient binding of the RNA editing entity to the engineered guide RNA. In some examples, the recruiting domain allows for permanent binding of the RNA editing entity to the engineered guide RNA. A recruiting domain can be of any length. In some cases, a recruiting domain can be from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, up to about 80 nucleotides in length. In some cases, a recruiting domain can be no more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or 80 nucleotides in length. In some cases, a recruiting domain can be about 45 nucleotides in length. In some cases, at least a portion of a recruiting domain comprises at least 1 to about 75 nucleotides. In some cases, at least a portion of a recruiting domain comprises about 45 nucleotides to about 60 nucleotides.

In some embodiments, a recruiting domain comprises a GluR2 sequence or functional fragment thereof. In some cases, a GluR2 sequence can be recognized by an RNA editing entity, such as an ADAR or biologically active fragment thereof. In some embodiments, a GluR2 sequence can be a non-naturally occurring sequence. In some cases, a GluR2 sequence can be modified, for example for enhanced recruitment. In some embodiments, a GluR2 sequence can comprise a portion of a naturally occurring GluR2 sequence and a synthetic sequence.

In some examples, a recruiting domain comprises a GluR2 sequence, or a sequence having at least about 70%, 80%, 85%, 90%, 95%, 98%, 99%, or 100% identity and/or length to: GUGGAAUAGUAUAACAAUAUGCUAAAUGUUGUUAUAGUAUCCCAC (SEQ ID NO: 1). In some cases, a recruiting domain can comprise at least about 80% sequence homology to at least about 10, 15, 20, 25, or 30 nucleotides of SEQ ID NO: 1. In some examples, a recruiting domain can comprise at least about 90%, 95%, 96%, 97%, 98%, or 99% sequence homology and/or length to SEQ ID NO: 1.

Any number of recruiting domains can be found in an engineered RNA of the present disclosure. In some examples, at least about 1, 2, 3, 4, 5, 6, 7, 8, 9, or up to about 10 recruiting domains can be included in an engineered RNA. Recruiting domains can be located at any position of an engineered guide RNA. In some cases, a recruiting domain can be on an N-terminus, middle, or C-terminus of a polynucleotide. A recruiting domain can be upstream or downstream of a targeting sequence. In some cases, a recruiting domain flanks a targeting sequence of a guide. A recruiting sequence can comprise all ribonucleotides or deoxyribonucleotides, although a recruiting domain comprising both ribo- and deoxyribonucleotides can in some cases not be excluded.

C. Engineered Guide RNAs with a Micro-Footprint Sequence Having Latent Structure

In some examples, an engineered guides disclosed herein useful for facilitating editing of a LRRK2 target RNA via an RNA editing entity (e.g., a guide RNA having a polynucleotide sequence of any one of SEQ ID NO: 2-SEQ ID NO: 395 or SEQ ID NO: 398-SEQ ID NO: 472) can be an engineered latent guide RNA. An “engineered latent guide RNA” refers to an engineered guide RNA that comprises latent structure. A micro-footprint sequence of a guide RNA comprising latent structures (e.g., a “latent structure guide RNA”) can comprise a portion of sequence that, upon hybridization to a target RNA, forms at least a portion of a structural feature, other than a single A/C mismatch feature at the target adenosine to be edited. “Latent structure” refers to a structural feature that substantially forms upon hybridization of a guide RNA to a target RNA. For example, the sequence of a guide RNA provides one or more structural features, but these structural features substantially form only upon hybridization to the target RNA, and thus the one or more latent structural features manifest as structural features upon hybridization to the target RNA. Upon hybridization of the guide RNA to the target RNA, the structural feature is formed and the latent structure provided in the guide RNA is, thus, unmasked.

A double stranded RNA (dsRNA) substrate is formed upon hybridization of an engineered guide RNA of the present disclosure to a target RNA. The resulting dsRNA substrate is also referred to herein as a “guide-target RNA scaffold.”

In some embodiments, the present disclosure provides for engineered guide RNAs comprising a barbell macro-footprint. In some embodiments, the present disclosure provides for engineered guide RNAs comprising a micro-footprint. In some embodiments, the present disclosure provides for engineered guide RNAs comprising a macro-footprint and a micro-footprint, where the macro-footprint includes barbells (or internal loops) near the 5′ and 3′ ends of the guide-target RNA scaffold and the micro-footprint includes other structural features including, but not limited to, mismatches, symmetric internal loops, asymmetric internal loops, symmetric bulges, or asymmetric bulges. For example, an engineered guide RNA disclosed herein can have a macro-footprint and a micro-footprint of A/G mismatches at local off-target adenosines. An engineered guide RNA disclosed herein may have a macro-footprint and a micro-footprint of 1/0 asymmetric bulges (formed by an A in the target RNA and deletion of a U in the engineered guide RNA) at local off-target adenosines. An engineered guide RNA disclosed herein can have a macro-footprint of barbells (including an internal loop near the 5′ end of the guide-target RNA scaffold and an internal loop near the 3′ end of the guide-target RNA scaffold) and a micro-footprint of A/G mismatches at local off-target adenosines. An engineered guide RNA disclosed herein may have a macro-footprint of barbells (including an internal loop near the 5′ end of the guide-target RNA scaffold and an internal loop near the 3′ end of the guide-target RNA scaffold) and a micro-footprint of 1/0 asymmetric bulges (formed by an A in the target RNA and deletion of a U in the engineered guide RNA) at local off-target adenosines. In some embodiments, an engineered guide RNA disclosed herein may have a macro-footprint of barbells (including an internal loop near the 5′ end of the guide-target RNA scaffold and an internal loop near the 3′ end of the guide-target RNA scaffold) and a micro-footprint of a 5/5 symmetric loop, 1/1 G/G mismatch, and a 3/3 symmetric bulge to boost on-target adenosine editing while also reducing local off-target adenosine editing. In some embodiments, the barbell macro-footprint is engineered to form an internal loop at the −14 position and an internal loop at the +22 position relative to the target adenosine (position 0). In some embodiments, the barbell macro-footprint is engineered to form an internal loop at the −20 position and an internal loop at the +26 position relative to the target adenosine (position 0).

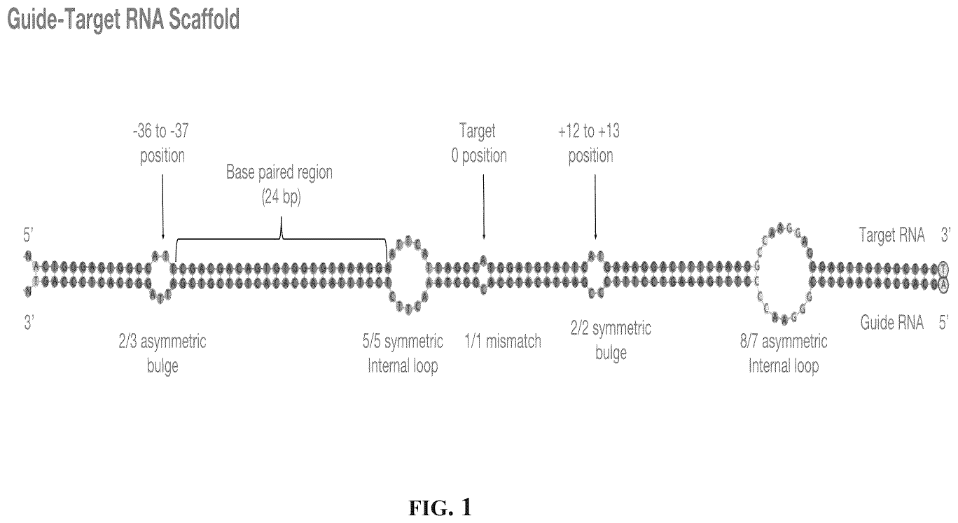

FIG. 1 shows a legend of various exemplary structural features present in guide-target RNA scaffolds formed upon hybridization of a latent guide RNA of the present disclosure to a target RNA. Example structural features shown include an 8/7 asymmetric loop (8 nucleotides on the target RNA side and 7 nucleotides on the guide RNA side), a 2/2 symmetric bulge (2 nucleotides on the target RNA side and 2 nucleotides on the guide RNA side), a 1/1 mismatch (1 nucleotide on the target RNA side and 1 nucleotide on the guide RNA side), a 5/5 symmetric internal loop (5 nucleotides on the target RNA side and 5 nucleotides on the guide RNA side), a 24 bp region (24 nucleotides on the target RNA side base paired to 24 nucleotides on the guide RNA side), and a 2/3 asymmetric bulge (2 nucleotides on the target RNA side and 3 nucleotides on the guide RNA side). Unless otherwise noted, the number of participating nucleotides in a given structural feature is indicated as the nucleotides on the target RNA side over nucleotides on the guide RNA side. Also shown in this legend is a key to the positional annotation of each figure. For example, the target nucleotide to be edited is designated as the 0 position. Downstream (3′) of the target nucleotide to be edited, each nucleotide is counted in increments of +1. Upstream (5′) of the target nucleotide to be edited, each nucleotide is counted in increments of −1. Thus, the example 2/2 symmetric bulge in this legend is at the +12 to +13 position in the guide-target RNA scaffold. Similarly, the 2/3 asymmetric bulge in this legend is at the −36 to −37 position in the guide-target RNA scaffold. As used herein, positional annotation is provided with respect to the target nucleotide to be edited and on the target RNA side of the guide-target RNA scaffold. As used herein, if a single position is annotated, the structural feature extends from that position away from position 0 (target nucleotide to be edited). For example, if a latent guide RNA is annotated herein as forming a 2/3 asymmetric bulge at position −36, then the 2/3 asymmetric bulge forms from −36 position to the −37 position with respect to the target nucleotide to be edited (position 0) on the target RNA side of the guide-target RNA scaffold. As another example, if a latent guide RNA is annotated herein as forming a 2/2 symmetric bulge at position +12, then the 2/2 symmetric bulge forms from the +12 to the +13 position with respect to the target nucleotide to be edited (position 0) on the target RNA side of the guide-target RNA scaffold.

In some instances, an engineered latent guide RNA lacks a recruiting domain, and recruitment of the RNA editing entity can be effectuated by structural features of the guide-target RNA scaffold formed by hybridization of the engineered guide RNA and the target RNA. In some examples, the engineered guide, when present in an aqueous solution and not bound to the target RNA molecule, does not comprise structural features that recruit the RNA editing entity (e.g., ADAR). The engineered latent guide RNA, upon hybridization to a target RNA, form with the target RNA molecule one or more structural features present in the guide-target RNA scaffold that recruits an RNA editing entity (e.g., ADAR).

Described herein are structural features which can be present in a guide-target RNA scaffold of the present disclosure. Examples of features include a mismatch, a bulge (symmetrical bulge or asymmetrical bulge), an internal loop (symmetrical internal loop or asymmetrical internal loop), or a hairpin (a recruiting hairpin or a non-recruiting hairpin). Engineered guide RNAs of the present disclosure can have from 1 to 50 features. Engineered guide RNAs of the present disclosure can have from 1 to 5, from 5 to 10, from 10 to 15, from 15 to 20, from 20 to 25, from 25 to 30, from 30 to 35, from 35 to 40, from 40 to 45, from 45 to 50, from 5 to 20, from 1 to 3, from 4 to 5, from 2 to 10, from 20 to 40, from 10 to 40, from 20 to 50, from 30 to 50, from 4 to 7, or from 8 to 10 features. In some embodiments, structural features (e.g., mismatches, bulges, internal loops) can be formed from latent structure in an engineered latent guide RNA upon hybridization of the engineered latent guide RNA to a target RNA and, thus, formation of a guide-target RNA scaffold. In some embodiments, structural features are not formed from latent structures and are, instead, pre-formed structures (e.g., a GluR2 recruitment hairpin or a hairpin from U7 snRNA).

A guide-target RNA scaffold is formed upon hybridization of an engineered guide RNA of the present disclosure to a target RNA. As disclosed herein, a mismatch refers to a single nucleotide in a guide RNA that is unpaired to an opposing single nucleotide in a target RNA within the guide-target RNA scaffold. A mismatch can comprise any two single nucleotides that do not base pair. Where the number of participating nucleotides on the guide RNA side and the target RNA side exceeds 1, the resulting structure is no longer considered a mismatch, but rather, is considered a bulge or an internal loop, depending on the size of the structural feature. In some embodiments, a mismatch is an A/C mismatch. An A/C mismatch can comprise a C in an engineered guide RNA of the present disclosure opposite an A in a target RNA. An A/C mismatch can comprise an A in an engineered guide RNA of the present disclosure opposite a C in a target RNA. A G/G mismatch can comprise a G in an engineered guide RNA of the present disclosure opposite a G in a target RNA.

In some embodiments, a mismatch positioned 5′ of the edit site can facilitate base-flipping of the target A to be edited. A mismatch can also help confer sequence specificity. Thus, a mismatch can be a structural feature formed from latent structure provided by an engineered latent guide RNA.

In another aspect, a structural feature comprises a wobble base. A wobble base pair refers to two bases that weakly base pair. For example, a wobble base pair of the present disclosure can refer to a G paired with a U. Thus, a wobble base pair can be a structural feature formed from latent structure provided by an engineered latent guide RNA.

In some cases, a structural feature can be a hairpin. As disclosed herein, a hairpin includes an RNA duplex wherein a portion of a single RNA strand has folded in upon itself to form the RNA duplex. The portion of the single RNA strand folds upon itself due to having nucleotide sequences that base pair to each other, where the nucleotide sequences are separated by an intervening sequence that does not base pair with itself, thus forming a base-paired portion and non-base paired, intervening loop portion. A hairpin can have from 10 to 500 nucleotides in length of the entire duplex structure. The loop portion of a hairpin can be from 3 to 15 nucleotides long. A hairpin can be present in any of the engineered guide RNAs disclosed herein. The engineered guide RNAs disclosed herein can have from 1 to 10 hairpins. In some embodiments, the engineered guide RNAs disclosed herein have 1 hairpin. In some embodiments, the engineered guide RNAs disclosed herein have 2 hairpins. As disclosed herein, a hairpin can include a recruitment hairpin or a non-recruitment hairpin. A hairpin can be located anywhere within the engineered guide RNAs of the present disclosure. In some embodiments, one or more hairpins is proximal to or present at the 3′ end of an engineered guide RNA of the present disclosure, proximal to or at the 5′ end of an engineered guide RNA of the present disclosure, proximal to or within the targeting domain of the engineered guide RNAs of the present disclosure, or any combination thereof.

In some aspects, a structural feature comprises a non-recruitment hairpin. A non-recruitment hairpin, as disclosed herein, does not have a primary function of recruiting an RNA editing entity. A non-recruitment hairpin, in some instances, does not recruit an RNA editing entity. In some instances, a non-recruitment hairpin has a dissociation constant for binding to an RNA editing entity under physiological conditions that is insufficient for binding. For example, a non-recruitment hairpin has a dissociation constant for binding an RNA editing entity at 25° C. that is greater than about 1 mM, 10 mM, 100 mM, or 1 M, as determined in an in vitro assay. A non-recruitment hairpin can exhibit functionality that improves localization of the engineered guide RNA to the target RNA. In some embodiments, the non-recruitment hairpin improves nuclear retention. In some embodiments, the non-recruitment hairpin comprises a hairpin from U7 snRNA. Thus, a non-recruitment hairpin such as a hairpin from U7 snRNA is a pre-formed structural feature that can be present in constructs comprising engineered guide RNA constructs, not a structural feature formed by latent structure provided in an engineered latent guide RNA.

A hairpin of the present disclosure can be of any length. In an aspect, a hairpin can be from about 10-500 or more nucleotides. In some cases, a hairpin can comprise about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 or more nucleotides. In other cases, a hairpin can also comprise 10 to 20, 10 to 30, 10 to 40, 10 to 50, 10 to 60, 10 to 70, 10 to 80, 10 to 90, 10 to 100, 10 to 110, 10 to 120, 10 to 130, 10 to 140, 10 to 150, 10 to 160, 10 to 170, 10 to 180, 10 to 190, 10 to 200, 10 to 210, 10 to 220, 10 to 230, 10 to 240, 10 to 250, 10 to 260, 10 to 270, 10 to 280, 10 to 290, 10 to 300, 10 to 310, 10 to 320, 10 to 330, 10 to 340, 10 to 350, 10 to 360, 10 to 370, 10 to 380, 10 to 390, 10 to 400, 10 to 410, 10 to 420, 10 to 430, 10 to 440, 10 to 450, 10 to 460, 10 to 470, 10 to 480, 10 to 490, or 10 to 500 nucleotides.

A guide-target RNA scaffold is formed upon hybridization of an engineered guide RNA of the present disclosure to a target RNA. As disclosed herein, a bulge refers to the structure substantially formed only upon formation of the guide-target RNA scaffold, where contiguous nucleotides in either the engineered guide RNA or the target RNA are not complementary to their positional counterparts on the opposite strand. A bulge can change the secondary or tertiary structure of the guide-target RNA scaffold. A bulge can independently have from 0 to 4 contiguous nucleotides on the guide RNA side of the guide-target RNA scaffold and 1 to 4 contiguous nucleotides on the target RNA side of the guide-target RNA scaffold or a bulge can independently have from 0 to 4 nucleotides on the target RNA side of the guide-target RNA scaffold and 1 to 4 contiguous nucleotides on the guide RNA side of the guide-target RNA scaffold. However, a bulge, as used herein, does not refer to a structure where a single participating nucleotide of the engineered guide RNA and a single participating nucleotide of the target RNA do not base pair—a single participating nucleotide of the engineered guide RNA and a single participating nucleotide of the target RNA that do not base pair is referred to herein as a mismatch. Further, where the number of participating nucleotides on either the guide RNA side or the target RNA side exceeds 4, the resulting structure is no longer considered a bulge, but rather, is considered an internal loop. In some embodiments, the guide-target RNA scaffold of the present disclosure has 2 bulges. In some embodiments, the guide-target RNA scaffold of the present disclosure has 3 bulges. In some embodiments, the guide-target RNA scaffold of the present disclosure has 4 bulges. Thus, a bulge can be a structural feature formed from latent structure provided by an engineered latent guide RNA.

In some embodiments, the presence of a bulge in a guide-target RNA scaffold can position or can help to position ADAR to selectively edit the target A in the target RNA and reduce off-target editing of non-target A(s) in the target RNA. In some embodiments, the presence of a bulge in a guide-target RNA scaffold can recruit or help recruit additional amounts of ADAR. Bulges in guide-target RNA scaffolds disclosed herein can recruit other proteins, such as other RNA editing entities. In some embodiments, a bulge positioned 5′ of the edit site can facilitate base-flipping of the target A to be edited. A bulge can also help confer sequence specificity for the A of the target RNA to be edited, relative to other A(s) present in the target RNA. For example, a bulge can help direct ADAR editing by constraining it in an orientation that yields selective editing of the target A.

A guide-target RNA scaffold is formed upon hybridization of an engineered guide RNA of the present disclosure to a target RNA. A bulge can be a symmetrical bulge or an asymmetrical bulge. A symmetrical bulge is formed when the same number of nucleotides is present on each side of the bulge. For example, a symmetrical bulge in a guide-target RNA scaffold of the present disclosure can have the same number of nucleotides on the engineered guide RNA side and the target RNA side of the guide-target RNA scaffold. A symmetrical bulge of the present disclosure can be formed by 2 nucleotides on the engineered guide RNA side of the guide-target RNA scaffold target and 2 nucleotides on the target RNA side of the guide-target RNA scaffold. A symmetrical bulge of the present disclosure can be formed by 3 nucleotides on the engineered guide RNA side of the guide-target RNA scaffold target and 3 nucleotides on the target RNA side of the guide-target RNA scaffold. A symmetrical bulge of the present disclosure can be formed by 4 nucleotides on the engineered guide RNA side of the guide-target RNA scaffold target and 4 nucleotides on the target RNA side of the guide-target RNA scaffold. Thus, a symmetrical bulge can be a structural feature formed from latent structure provided by an engineered latent guide RNA.

A guide-target RNA scaffold is formed upon hybridization of an engineered guide RNA of the present disclosure to a target RNA. A bulge can be a symmetrical bulge or an asymmetrical bulge. An asymmetrical bulge is formed when a different number of nucleotides is present on each side of the bulge. For example, an asymmetrical bulge in a guide-target RNA scaffold of the present disclosure can have different numbers of nucleotides on the engineered guide RNA side and the target RNA side of the guide-target RNA scaffold. An asymmetrical bulge of the present disclosure can be formed by 0 nucleotides on the engineered guide RNA side of the guide-target RNA scaffold and 1 nucleotide on the target RNA side of the guide-target RNA scaffold. An asymmetrical bulge of the present disclosure can be formed by 0 nucleotides on the target RNA side of the guide-target RNA scaffold and 1 nucleotide on the engineered guide RNA side of the guide-target RNA scaffold. An asymmetrical bulge of the present disclosure can be formed by 0 nucleotides on the engineered guide RNA side of the guide-target RNA scaffold and 2 nucleotides on the target RNA side of the guide-target RNA scaffold. An asymmetrical bulge of the present disclosure can be formed by 0 nucleotides on the target RNA side of the guide-target RNA scaffold and 2 nucleotides on the engineered guide RNA side of the guide-target RNA scaffold. An asymmetrical bulge of the present disclosure can be formed by 0 nucleotides on the engineered guide RNA side of the guide-target RNA scaffold and 3 nucleotides on the target RNA side of the guide-target RNA scaffold. An asymmetrical bulge of the present disclosure can be formed by 0 nucleotides on the target RNA side of the guide-target RNA scaffold and 3 nucleotides on the engineered guide RNA side of the guide-target RNA scaffold. An asymmetrical bulge of the present disclosure can be formed by 0 nucleotides on the engineered guide RNA side of the guide-target RNA scaffold and 4 nucleotides on the target RNA side of the guide-target RNA scaffold. An asymmetrical bulge of the present disclosure can be formed by 0 nucleotides on the target RNA side of the guide-target RNA scaffold and 4 nucleotides on the engineered guide RNA side of the guide-target RNA scaffold. An asymmetrical bulge of the present disclosure can be formed by 1 nucleotide on the engineered guide RNA side of the guide-target RNA scaffold and 2 nucleotides on the target RNA side of the guide-target RNA scaffold. An asymmetrical bulge of the present disclosure can be formed by 1 nucleotide on the target RNA side of the guide-target RNA scaffold and 2 nucleotides on the engineered guide RNA side of the guide-target RNA scaffold. An asymmetrical bulge of the present disclosure can be formed by 1 nucleotide on the engineered guide RNA side of the guide-target RNA scaffold and 3 nucleotides on the target RNA side of the guide-target RNA scaffold. An asymmetrical bulge of the present disclosure can be formed by 1 nucleotide on the target RNA side of the guide-target RNA scaffold and 3 nucleotides on the engineered guide RNA side of the guide-target RNA scaffold. An asymmetrical bulge of the present disclosure can be formed by 1 nucleotide on the engineered guide RNA side of the guide-target RNA scaffold and 4 nucleotides on the target RNA side of the guide-target RNA scaffold. An asymmetrical bulge of the present disclosure can be formed by 1 nucleotide on the target RNA side of the guide-target RNA scaffold and 4 nucleotides on the engineered guide RNA side of the guide-target RNA scaffold. An asymmetrical bulge of the present disclosure can be formed by 2 nucleotides on the engineered guide RNA side of the guide-target RNA scaffold and 3 nucleotides on the target RNA side of the guide-target RNA scaffold. An asymmetrical bulge of the present disclosure can be formed by 2 nucleotides on the target RNA side of the guide-target RNA scaffold and 3 nucleotides on the engineered guide RNA side of the guide-target RNA scaffold. An asymmetrical bulge of the present disclosure can be formed by 2 nucleotides on the engineered guide RNA side of the guide-target RNA scaffold and 4 nucleotides on the target RNA side of the guide-target RNA scaffold. An asymmetrical bulge of the present disclosure can be formed by 2 nucleotides on the target RNA side of the guide-target RNA scaffold and 4 nucleotides on the engineered guide RNA side of the guide-target RNA scaffold. An asymmetrical bulge of the present disclosure can be formed by 3 nucleotides on the engineered guide RNA side of the guide-target RNA scaffold and 4 nucleotides on the target RNA side of the guide-target RNA scaffold. An asymmetrical bulge of the present disclosure can be formed by 3 nucleotides on the target RNA side of the guide-target RNA scaffold and 4 nucleotides on the engineered guide RNA side of the guide-target RNA scaffold. Thus, an asymmetrical bulge can be a structural feature formed from latent structure provided by an engineered latent guide RNA.

In some cases, a structural feature can be an internal loop. As disclosed herein, an internal loop refers to the structure substantially formed only upon formation of the guide-target RNA scaffold, where nucleotides in either the engineered guide RNA or the target RNA are not complementary to their positional counterparts on the opposite strand and where one side of the internal loop, either on the target RNA side or the engineered guide RNA side of the guide-target RNA scaffold, has 5 nucleotides or more. Where the number of participating nucleotides on both the guide RNA side and the target RNA side drops below 5, the resulting structure is no longer considered an internal loop, but rather, is considered a bulge or a mismatch, depending on the size of the structural feature. An internal loop can be a symmetrical internal loop or an asymmetrical internal loop. Internal loops present in the vicinity of the edit site can help with base flipping of the target A in the target RNA to be edited.

One side of the internal loop, either on the target RNA side or the engineered guide RNA side of the guide-target RNA scaffold, can be formed by from 5 to 150 nucleotides. One side of the internal loop can be formed by 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 120, 135, 140, 145, 150, 200, 250, 300, 350, 400, 450, 500, 600, 700, 800, 900, or 1000 nucleotides, or any number of nucleotides therebetween. One side of the internal loop can be formed by 5 nucleotides. One side of the internal loop can be formed by 10 nucleotides. One side of the internal loop can be formed by 15 nucleotides. One side of the internal loop can be formed by 20 nucleotides. One side of the internal loop can be formed by 25 nucleotides. One side of the internal loop can be formed by 30 nucleotides. One side of the internal loop can be formed by 35 nucleotides. One side of the internal loop can be formed by 40 nucleotides. One side of the internal loop can be formed by 45 nucleotides. One side of the internal loop can be formed by 50 nucleotides. One side of the internal loop can be formed by 55 nucleotides. One side of the internal loop can be formed by 60 nucleotides. One side of the internal loop can be formed by 65 nucleotides. One side of the internal loop can be formed by 70 nucleotides. One side of the internal loop can be formed by 75 nucleotides. One side of the internal loop can be formed by 80 nucleotides. One side of the internal loop can be formed by 85 nucleotides. One side of the internal loop can be formed by 90 nucleotides. One side of the internal loop can be formed by 95 nucleotides. One side of the internal loop can be formed by 100 nucleotides. One side of the internal loop can be formed by 110 nucleotides. One side of the internal loop can be formed by 120 nucleotides. One side of the internal loop can be formed by 130 nucleotides. One side of the internal loop can be formed by 140 nucleotides. One side of the internal loop can be formed by 150 nucleotides. One side of the internal loop can be formed by 200 nucleotides. One side of the internal loop can be formed by 250 nucleotides. One side of the internal loop can be formed by 300 nucleotides. One side of the internal loop can be formed by 350 nucleotides. One side of the internal loop can be formed by 400 nucleotides. One side of the internal loop can be formed by 450 nucleotides. One side of the internal loop can be formed by 500 nucleotides. One side of the internal loop can be formed by 600 nucleotides. One side of the internal loop can be formed by 700 nucleotides. One side of the internal loop can be formed by 800 nucleotides. One side of the internal loop can be formed by 900 nucleotides. One side of the internal loop can be formed by 1000 nucleotides. Thus, an internal loop can be a structural feature formed from latent structure provided by an engineered latent guide RNA.

An internal loop can be a symmetrical internal loop or an asymmetrical internal loop. A symmetrical internal loop is formed when the same number of nucleotides is present on each side of the internal loop. For example, a symmetrical internal loop in a guide-target RNA scaffold of the present disclosure can have the same number of nucleotides on the engineered guide RNA side and the target RNA side of the guide-target RNA scaffold. A symmetrical internal loop of the present disclosure can be formed by 5 nucleotides on the engineered guide RNA side of the guide-target RNA scaffold target and 5 nucleotides on the target RNA side of the guide-target RNA scaffold. A symmetrical internal loop of the present disclosure can be formed by 6 nucleotides on the engineered guide RNA side of the guide-target RNA scaffold target and 6 nucleotides on the target RNA side of the guide-target RNA scaffold. A symmetrical internal loop of the present disclosure can be formed by 7 nucleotides on the engineered guide RNA side of the guide-target RNA scaffold target and 7 nucleotides on the target RNA side of the guide-target RNA scaffold. A symmetrical internal loop of the present disclosure can be formed by 8 nucleotides on the engineered guide RNA side of the guide-target RNA scaffold target and 8 nucleotides on the target RNA side of the guide-target RNA scaffold. A symmetrical internal loop of the present disclosure can be formed by 9 nucleotides on the engineered guide RNA side of the guide-target RNA scaffold target and 9 nucleotides on the target RNA side of the guide-target RNA scaffold. A symmetrical internal loop of the present disclosure can be formed by 10 nucleotides on the engineered guide RNA side of the guide-target RNA scaffold target and 10 nucleotides on the target RNA side of the guide-target RNA scaffold. A symmetrical internal loop of the present disclosure can be formed by 15 nucleotides on the engineered guide RNA side of the guide-target RNA scaffold target and 15 nucleotides on the target RNA side of the guide-target RNA scaffold. A symmetrical internal loop of the present disclosure can be formed by 20 nucleotides on the engineered guide RNA side of the guide-target RNA scaffold target and 20 nucleotides on the target RNA side of the guide-target RNA scaffold. A symmetrical internal loop of the present disclosure can be formed by 30 nucleotides on the engineered guide RNA side of the guide-target RNA scaffold target and 30 nucleotides on the target RNA side of the guide-target RNA scaffold. A symmetrical internal loop of the present disclosure can be formed by 40 nucleotides on the engineered guide RNA side of the guide-target RNA scaffold target and 40 nucleotides on the target RNA side of the guide-target RNA scaffold. A symmetrical internal loop of the present disclosure can be formed by 50 nucleotides on the engineered guide RNA side of the guide-target RNA scaffold target and 50 nucleotides on the target RNA side of the guide-target RNA scaffold. A symmetrical internal loop of the present disclosure can be formed by 60 nucleotides on the engineered guide RNA side of the guide-target RNA scaffold target and 60 nucleotides on the target RNA side of the guide-target RNA scaffold. A symmetrical internal loop of the present disclosure can be formed by 70 nucleotides on the engineered guide RNA side of the guide-target RNA scaffold target and 70 nucleotides on the target RNA side of the guide-target RNA scaffold. A symmetrical internal loop of the present disclosure can be formed by 80 nucleotides on the engineered guide RNA side of the guide-target RNA scaffold target and 80 nucleotides on the target RNA side of the guide-target RNA scaffold. A symmetrical internal loop of the present disclosure can be formed by 90 nucleotides on the engineered guide RNA side of the guide-target RNA scaffold target and 90 nucleotides on the target RNA side of the guide-target RNA scaffold. A symmetrical internal loop of the present disclosure can be formed by 100 nucleotides on the engineered guide RNA side of the guide-target RNA scaffold target and 100 nucleotides on the target RNA side of the guide-target RNA scaffold. A symmetrical internal loop of the present disclosure can be formed by 110 nucleotides on the engineered guide RNA side of the guide-target RNA scaffold target and 110 nucleotides on the target RNA side of the guide-target RNA scaffold. A symmetrical internal loop of the present disclosure can be formed by 120 nucleotides on the engineered guide RNA side of the guide-target RNA scaffold target and 120 nucleotides on the target RNA side of the guide-target RNA scaffold. A symmetrical internal loop of the present disclosure can be formed by 130 nucleotides on the engineered guide RNA side of the guide-target RNA scaffold target and 130 nucleotides on the target RNA side of the guide-target RNA scaffold. A symmetrical internal loop of the present disclosure can be formed by 140 nucleotides on the engineered guide RNA side of the guide-target RNA scaffold target and 140 nucleotides on the target RNA side of the guide-target RNA scaffold. A symmetrical internal loop of the present disclosure can be formed by 150 nucleotides on the engineered guide RNA side of the guide-target RNA scaffold target and 150 nucleotides on the target RNA side of the guide-target RNA scaffold. A symmetrical internal loop of the present disclosure can be formed by 200 nucleotides on the engineered guide RNA side of the guide-target RNA scaffold target and 200 nucleotides on the target RNA side of the guide-target RNA scaffold. A symmetrical internal loop of the present disclosure can be formed by 250 nucleotides on the engineered guide RNA side of the guide-target RNA scaffold target and 250 nucleotides on the target RNA side of the guide-target RNA scaffold. A symmetrical internal loop of the present disclosure can be formed by 300 nucleotides on the engineered guide RNA side of the guide-target RNA scaffold target and 300 nucleotides on the target RNA side of the guide-target RNA scaffold. A symmetrical internal loop of the present disclosure can be formed by 350 nucleotides on the engineered guide RNA side of the guide-target RNA scaffold target and 350 nucleotides on the target RNA side of the guide-target RNA scaffold. A symmetrical internal loop of the present disclosure can be formed by 400 nucleotides on the engineered guide RNA side of the guide-target RNA scaffold target and 400 nucleotides on the target RNA side of the guide-target RNA scaffold. A symmetrical internal loop of the present disclosure can be formed by 450 nucleotides on the engineered guide RNA side of the guide-target RNA scaffold target and 450 nucleotides on the target RNA side of the guide-target RNA scaffold. A symmetrical internal loop of the present disclosure can be formed by 500 nucleotides on the engineered guide RNA side of the guide-target RNA scaffold target and 500 nucleotides on the target RNA side of the guide-target RNA scaffold. A symmetrical internal loop of the present disclosure can be formed by 600 nucleotides on the engineered guide RNA side of the guide-target RNA scaffold target and 600 nucleotides on the target RNA side of the guide-target RNA scaffold. A symmetrical internal loop of the present disclosure can be formed by 700 nucleotides on the engineered guide RNA side of the guide-target RNA scaffold target and 700 nucleotides on the target RNA side of the guide-target RNA scaffold. A symmetrical internal loop of the present disclosure can be formed by 800 nucleotides on the engineered guide RNA side of the guide-target RNA scaffold target and 800 nucleotides on the target RNA side of the guide-target RNA scaffold. A symmetrical internal loop of the present disclosure can be formed by 900 nucleotides on the engineered guide RNA side of the guide-target RNA scaffold target and 900 nucleotides on the target RNA side of the guide-target RNA scaffold. A symmetrical internal loop of the present disclosure can be formed by 1000 nucleotides on the engineered guide RNA side of the guide-target RNA scaffold target and 1000 nucleotides on the target RNA side of the guide-target RNA scaffold. Thus, a symmetrical internal loop can be a structural feature formed from latent structure provided by an engineered latent guide RNA.

An asymmetrical internal loop is formed when a different number of nucleotides is present on each side of the internal loop. For example, an asymmetrical internal loop in a guide-target RNA scaffold of the present disclosure can have different numbers of nucleotides on the engineered guide RNA side and the target RNA side of the guide-target RNA scaffold.

An asymmetrical internal loop of the present disclosure can be formed by from 5 to 150 nucleotides on the engineered guide RNA side of the guide-target RNA scaffold and from 5 to 150 nucleotides on the target RNA side of the guide-target RNA scaffold, wherein the number of nucleotides is the different on the engineered side of the guide-target RNA scaffold target than the number of nucleotides on the target RNA side of the guide-target RNA scaffold. An asymmetrical internal loop of the present disclosure can be formed by from 5 to 1000 nucleotides on the engineered guide RNA side of the guide-target RNA scaffold and from 5 to 1000 nucleotides on the target RNA side of the guide-target RNA scaffold, wherein the number of nucleotides is the different on the engineered side of the guide-target RNA scaffold target than the number of nucleotides on the target RNA side of the guide-target RNA scaffold. An asymmetrical internal loop of the present disclosure can be formed by 5 nucleotides on the engineered guide RNA side of the guide-target RNA scaffold and 6 nucleotides on the target RNA side of the guide-target RNA scaffold. An asymmetrical internal loop of the present disclosure can be formed by 5 nucleotides on the target RNA side of the guide-target RNA scaffold and 6 nucleotides on the engineered guide RNA side of the guide-target RNA scaffold. An asymmetrical internal loop of the present disclosure can be formed by 5 nucleotides on the engineered guide RNA side of the guide-target RNA scaffold and 7 nucleotides on the target RNA side of the guide-target RNA scaffold. An asymmetrical internal loop of the present disclosure can be formed by 5 nucleotides on the target RNA side of the guide-target RNA scaffold and 7 nucleotides on the engineered guide RNA side of the guide-target RNA scaffold. An asymmetrical internal loop of the present disclosure can be formed by 5 nucleotides on the engineered guide RNA side of the guide-target RNA scaffold and 8 nucleotides internal loop the target RNA side of the guide-target RNA scaffold. An asymmetrical internal loop of the present disclosure can be formed by 5 nucleotides on the target RNA side of the guide-target RNA scaffold and 8 nucleotides on the engineered guide RNA side of the guide-target RNA scaffold. An asymmetrical internal loop of the present disclosure can be formed by 5 nucleotides on the engineered guide RNA side of the guide-target RNA scaffold and 9 nucleotides internal loop the target RNA side of the guide-target RNA scaffold. An asymmetrical internal loop of the present disclosure can be formed by 5 nucleotides on the target RNA side of the guide-target RNA scaffold and 9 nucleotides on the engineered guide RNA side of the guide-target RNA scaffold. An asymmetrical internal loop of the present disclosure can be formed by 5 nucleotides on the engineered guide RNA side of the guide-target RNA scaffold and 10 nucleotides internal loop the target RNA side of the guide-target RNA scaffold. An asymmetrical internal loop of the present disclosure can be formed by 5 nucleotides on the target RNA side of the guide-target RNA scaffold and 10 nucleotides on the engineered guide RNA side of the guide-target RNA scaffold. An asymmetrical internal loop of the present disclosure can be formed by 6 nucleotides on the engineered guide RNA side of the guide-target RNA scaffold and 7 nucleotides internal loop the target RNA side of the guide-target RNA scaffold. An asymmetrical internal loop of the present disclosure can be formed by 6 nucleotides on the target RNA side of the guide-target RNA scaffold and 7 nucleotides on the engineered guide RNA side of the guide-target RNA scaffold. An asymmetrical internal loop of the present disclosure can be formed by 6 nucleotides on the engineered guide RNA side of the guide-target RNA scaffold and 8 nucleotides internal loop the target RNA side of the guide-target RNA scaffold. An asymmetrical internal loop of the present disclosure can be formed by 6 nucleotides on the target RNA side of the guide-target RNA scaffold and 8 nucleotides on the engineered guide RNA side of the guide-target RNA scaffold. An asymmetrical internal loop of the present disclosure can be formed by 6 nucleotides on the engineered guide RNA side of the guide-target RNA scaffold and 9 nucleotides internal loop the target RNA side of the guide-target RNA scaffold. An asymmetrical internal loop of the present disclosure can be formed by 6 nucleotides on the target RNA side of the guide-target RNA scaffold and 9 nucleotides on the engineered guide RNA side of the guide-target RNA scaffold. An asymmetrical internal loop of the present disclosure can be formed by 6 nucleotides on the engineered guide RNA side of the guide-target RNA scaffold and 10 nucleotides internal loop the target RNA side of the guide-target RNA scaffold. An asymmetrical internal loop of the present disclosure can be formed by 6 nucleotides on the target RNA side of the guide-target RNA scaffold and 10 nucleotides on the engineered guide RNA side of the guide-target RNA scaffold. An asymmetrical internal loop of the present disclosure can be formed by 7 nucleotides on the engineered guide RNA side of the guide-target RNA scaffold and 8 nucleotides internal loop the target RNA side of the guide-target RNA scaffold. An asymmetrical internal loop of the present disclosure can be formed by 7 nucleotides on the target RNA side of the guide-target RNA scaffold and 8 nucleotides on the engineered guide RNA side of the guide-target RNA scaffold. An asymmetrical internal loop of the present disclosure can be formed by 7 nucleotides on the engineered guide RNA side of the guide-target RNA scaffold and 9 nucleotides internal loop the target RNA side of the guide-target RNA scaffold. An asymmetrical internal loop of the present disclosure can be formed by 7 nucleotides on the target RNA side of the guide-target RNA scaffold and 9 nucleotides on the engineered guide RNA side of the guide-target RNA scaffold. An asymmetrical internal loop of the present disclosure can be formed by 7 nucleotides on the engineered guide RNA side of the guide-target RNA scaffold and 10 nucleotides internal loop the target RNA side of the guide-target RNA scaffold. An asymmetrical internal loop of the present disclosure can be formed by 7 nucleotides on the target RNA side of the guide-target RNA scaffold and 10 nucleotides on the engineered guide RNA side of the guide-target RNA scaffold. An asymmetrical internal loop of the present disclosure can be formed by 8 nucleotides on the engineered guide RNA side of the guide-target RNA scaffold and 9 nucleotides internal loop the target RNA side of the guide-target RNA scaffold. An asymmetrical internal loop of the present disclosure can be formed by 8 nucleotides on the target RNA side of the guide-target RNA scaffold and 9 nucleotides on the engineered guide RNA side of the guide-target RNA scaffold. An asymmetrical internal loop of the present disclosure can be formed by 8 nucleotides on the engineered guide RNA side of the guide-target RNA scaffold and 10 nucleotides internal loop the target RNA side of the guide-target RNA scaffold. An asymmetrical internal loop of the present disclosure can be formed by 8 nucleotides on the target RNA side of the guide-target RNA scaffold and 10 nucleotides on the engineered guide RNA side of the guide-target RNA scaffold. An asymmetrical internal loop of the present disclosure can be formed by 9 nucleotides on the engineered guide RNA side of the guide-target RNA scaffold and 10 nucleotides internal loop the target RNA side of the guide-target RNA scaffold. An asymmetrical internal loop of the present disclosure can be formed by 9 nucleotides on the target RNA side of the guide-target RNA scaffold and 10 nucleotides on the engineered guide RNA side of the guide-target RNA scaffold. An asymmetrical internal loop of the present disclosure can be formed by 5 nucleotides on the target RNA side of the guide-target RNA scaffold and 50 nucleotides on the engineered guide RNA side of the guide-target RNA scaffold. An asymmetrical internal loop of the present disclosure can be formed by 5 nucleotides on the target RNA side of the guide-target RNA scaffold and 100 nucleotides on the engineered guide RNA side of the guide-target RNA scaffold. An asymmetrical internal loop of the present disclosure can be formed by 5 nucleotides on the target RNA side of the guide-target RNA scaffold and 150 nucleotides on the engineered guide RNA side of the guide-target RNA scaffold. An asymmetrical internal loop of the present disclosure can be formed by 5 nucleotides on the target RNA side of the guide-target RNA scaffold and 200 nucleotides on the engineered guide RNA side of the guide-target RNA scaffold. An asymmetrical internal loop of the present disclosure can be formed by 5 nucleotides on the target RNA side of the guide-target RNA scaffold and 300 nucleotides on the engineered guide RNA side of the guide-target RNA scaffold. An asymmetrical internal loop of the present disclosure can be formed by 5 nucleotides on the target RNA side of the guide-target RNA scaffold and 400 nucleotides on the engineered guide RNA side of the guide-target RNA scaffold. An asymmetrical internal loop of the present disclosure can be formed by 5 nucleotides on the target RNA side of the guide-target RNA scaffold and 500 nucleotides on the engineered guide RNA side of the guide-target RNA scaffold. An asymmetrical internal loop of the present disclosure can be formed by 5 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 5 nucleotides on the engineered guide RNA side of the guide-target RNA scaffold. An asymmetrical internal loop of the present disclosure can be formed by 500 nucleotides on the target RNA side of the guide-target RNA scaffold and 5 nucleotides on the engineered guide RNA side of the guide-target RNA scaffold. An asymmetrical internal loop of the present disclosure can be formed by 400 nucleotides on the target RNA side of the guide-target RNA scaffold and 5 nucleotides on the engineered guide RNA side of the guide-target RNA scaffold. An asymmetrical internal loop of the present disclosure can be formed by 300 nucleotides on the target RNA side of the guide-target RNA scaffold and 5 nucleotides on the engineered guide RNA side of the guide-target RNA scaffold. An asymmetrical internal loop of the present disclosure can be formed by 200 nucleotides on the target RNA side of the guide-target RNA scaffold and 5 nucleotides on the engineered guide RNA side of the guide-target RNA scaffold. An asymmetrical internal loop of the present disclosure can be formed by 150 nucleotides on the target RNA side of the guide-target RNA scaffold and 5 nucleotides on the engineered guide RNA side of the guide-target RNA scaffold. An asymmetrical internal loop of the present disclosure can be formed by 100 nucleotides on the target RNA side of the guide-target RNA scaffold and 5 nucleotides on the engineered guide RNA side of the guide-target RNA scaffold. An asymmetrical internal loop of the present disclosure can be formed by 50 nucleotides on the target RNA side of the guide-target RNA scaffold and 5 nucleotides on the engineered guide RNA side of the guide-target RNA scaffold. An asymmetrical internal loop of the present disclosure can be formed by 50 nucleotides on the target RNA side of the guide-target RNA scaffold and 100 nucleotides on the engineered guide RNA side of the guide-target RNA scaffold. An asymmetrical internal loop of the present disclosure can be formed by 50 nucleotides on the target RNA side of the guide-target RNA scaffold and 150 nucleotides on the engineered guide RNA side of the guide-target RNA scaffold. An asymmetrical internal loop of the present disclosure can be formed by 50 nucleotides on the target RNA side of the guide-target RNA scaffold and 200 nucleotides on the engineered guide RNA side of the guide-target RNA scaffold. An asymmetrical internal loop of the present disclosure can be formed by 50 nucleotides on the target RNA side of the guide-target RNA scaffold and 300 nucleotides on the engineered guide RNA side of the guide-target RNA scaffold. An asymmetrical internal loop of the present disclosure can be formed by 50 nucleotides on the target RNA side of the guide-target RNA scaffold and 400 nucleotides on the engineered guide RNA side of the guide-target RNA scaffold. An asymmetrical internal loop of the present disclosure can be formed by 50 nucleotides on the target RNA side of the guide-target RNA scaffold and 500 nucleotides on the engineered guide RNA side of the guide-target RNA scaffold. An asymmetrical internal loop of the present disclosure can be formed by 50 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 50 nucleotides on the engineered guide RNA side of the guide-target RNA scaffold. An asymmetrical internal loop of the present disclosure can be formed by 500 nucleotides on the target RNA side of the guide-target RNA scaffold and 50 nucleotides on the engineered guide RNA side of the guide-target RNA scaffold. An asymmetrical internal loop of the present disclosure can be formed by 400 nucleotides on the target RNA side of the guide-target RNA scaffold and 50 nucleotides on the engineered guide RNA side of the guide-target RNA scaffold. An asymmetrical internal loop of the present disclosure can be formed by 300 nucleotides on the target RNA side of the guide-target RNA scaffold and 50 nucleotides on the engineered guide RNA side of the guide-target RNA scaffold. An asymmetrical internal loop of the present disclosure can be formed by 200 nucleotides on the target RNA side of the guide-target RNA scaffold and 50 nucleotides on the engineered guide RNA side of the guide-target RNA scaffold. An asymmetrical internal loop of the present disclosure can be formed by 150 nucleotides on the target RNA side of the guide-target RNA scaffold and 50 nucleotides on the engineered guide RNA side of the guide-target RNA scaffold. An asymmetrical internal loop of the present disclosure can be formed by 100 nucleotides on the target RNA side of the guide-target RNA scaffold and 50 nucleotides on the engineered guide RNA side of the guide-target RNA scaffold. An asymmetrical internal loop of the present disclosure can be formed by 100 nucleotides on the target RNA side of the guide-target RNA scaffold and 150 nucleotides on the engineered guide RNA side of the guide-target RNA scaffold. An asymmetrical internal loop of the present disclosure can be formed by 100 nucleotides on the target RNA side of the guide-target RNA scaffold and 200 nucleotides on the engineered guide RNA side of the guide-target RNA scaffold. An asymmetrical internal loop of the present disclosure can be formed by 100 nucleotides on the target RNA side of the guide-target RNA scaffold and 300 nucleotides on the engineered guide RNA side of the guide-target RNA scaffold. An asymmetrical internal loop of the present disclosure can be formed by 100 nucleotides on the target RNA side of the guide-target RNA scaffold and 400 nucleotides on the engineered guide RNA side of the guide-target RNA scaffold. An asymmetrical internal loop of the present disclosure can be formed by 100 nucleotides on the target RNA side of the guide-target RNA scaffold and 500 nucleotides on the engineered guide RNA side of the guide-target RNA scaffold. An asymmetrical internal loop of the present disclosure can be formed by 100 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 100 nucleotides on the engineered guide RNA side of the guide-target RNA scaffold. An asymmetrical internal loop of the present disclosure can be formed by 500 nucleotides on the target RNA side of the guide-target RNA scaffold and 100 nucleotides on the engineered guide RNA side of the guide-target RNA scaffold. An asymmetrical internal loop of the present disclosure can be formed by 400 nucleotides on the target RNA side of the guide-target RNA scaffold and 100 nucleotides on the engineered guide RNA side of the guide-target RNA scaffold. An asymmetrical internal loop of the present disclosure can be formed by 300 nucleotides on the target RNA side of the guide-target RNA scaffold and 100 nucleotides on the engineered guide RNA side of the guide-target RNA scaffold. An asymmetrical internal loop of the present disclosure can be formed by 200 nucleotides on the target RNA side of the guide-target RNA scaffold and 100 nucleotides on the engineered guide RNA side of the guide-target RNA scaffold. An asymmetrical internal loop of the present disclosure can be formed by 150 nucleotides on the target RNA side of the guide-target RNA scaffold and 100 nucleotides on the engineered guide RNA side of the guide-target RNA scaffold. An asymmetrical internal loop of the present disclosure can be formed by 150 nucleotides on the target RNA side of the guide-target RNA scaffold and 200 nucleotides on the engineered guide RNA side of the guide-target RNA scaffold. An asymmetrical internal loop of the present disclosure can be formed by 150 nucleotides on the target RNA side of the guide-target RNA scaffold and 300 nucleotides on the engineered guide RNA side of the guide-target RNA scaffold. An asymmetrical internal loop of the present disclosure can be formed by 150 nucleotides on the target RNA side of the guide-target RNA scaffold and 400 nucleotides on the engineered guide RNA side of the guide-target RNA scaffold. An asymmetrical internal loop of the present disclosure can be formed by 150 nucleotides on the target RNA side of the guide-target RNA scaffold and 500 nucleotides on the engineered guide RNA side of the guide-target RNA scaffold. An asymmetrical internal loop of the present disclosure can be formed by 150 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 150 nucleotides on the engineered guide RNA side of the guide-target RNA scaffold. An asymmetrical internal loop of the present disclosure can be formed by 500 nucleotides on the target RNA side of the guide-target RNA scaffold and 5 nucleotides on the engineered guide RNA side of the guide-target RNA scaffold. An asymmetrical internal loop of the present disclosure can be formed by 400 nucleotides on the target RNA side of the guide-target RNA scaffold and 150 nucleotides on the engineered guide RNA side of the guide-target RNA scaffold. An asymmetrical internal loop of the present disclosure can be formed by 300 nucleotides on the target RNA side of the guide-target RNA scaffold and 150 nucleotides on the engineered guide RNA side of the guide-target RNA scaffold. An asymmetrical internal loop of the present disclosure can be formed by 200 nucleotides on the target RNA side of the guide-target RNA scaffold and 300 nucleotides on the engineered guide RNA side of the guide-target RNA scaffold. An asymmetrical internal loop of the present disclosure can be formed by 200 nucleotides on the target RNA side of the guide-target RNA scaffold and 400 nucleotides on the engineered guide RNA side of the guide-target RNA scaffold. An asymmetrical internal loop of the present disclosure can be formed by 200 nucleotides on the target RNA side of the guide-target RNA scaffold and 500 nucleotides on the engineered guide RNA side of the guide-target RNA scaffold. An asymmetrical internal loop of the present disclosure can be formed by 200 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 200 nucleotides on the engineered guide RNA side of the guide-target RNA scaffold. An asymmetrical internal loop of the present disclosure can be formed by 500 nucleotides on the target RNA side of the guide-target RNA scaffold and 200 nucleotides on the engineered guide RNA side of the guide-target RNA scaffold. An asymmetrical internal loop of the present disclosure can be formed by 400 nucleotides on the target RNA side of the guide-target RNA scaffold and 200 nucleotides on the engineered guide RNA side of the guide-target RNA scaffold. An asymmetrical internal loop of the present disclosure can be formed by 300 nucleotides on the target RNA side of the guide-target RNA scaffold and 200 nucleotides on the engineered guide RNA side of the guide-target RNA scaffold. An asymmetrical internal loop of the present disclosure can be formed by 300 nucleotides on the target RNA side of the guide-target RNA scaffold and 400 nucleotides on the engineered guide RNA side of the guide-target RNA scaffold. An asymmetrical internal loop of the present disclosure can be formed by 300 nucleotides on the target RNA side of the guide-target RNA scaffold and 500 nucleotides on the engineered guide RNA side of the guide-target RNA scaffold. An asymmetrical internal loop of the present disclosure can be formed by 300 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 300 nucleotides on the engineered guide RNA side of the guide-target RNA scaffold. An asymmetrical internal loop of the present disclosure can be formed by 500 nucleotides on the target RNA side of the guide-target RNA scaffold and 300 nucleotides on the engineered guide RNA side of the guide-target RNA scaffold. An asymmetrical internal loop of the present disclosure can be formed by 400 nucleotides on the target RNA side of the guide-target RNA scaffold and 300 nucleotides on the engineered guide RNA side of the guide-target RNA scaffold. An asymmetrical internal loop of the present disclosure can be formed by 400 nucleotides on the target RNA side of the guide-target RNA scaffold and 500 nucleotides on the engineered guide RNA side of the guide-target RNA scaffold. An asymmetrical internal loop of the present disclosure can be formed by 400 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 400 nucleotides on the engineered guide RNA side of the guide-target RNA scaffold. An asymmetrical internal loop of the present disclosure can be formed by 500 nucleotides on the target RNA side of the guide-target RNA scaffold and 400 nucleotides on the engineered guide RNA side of the guide-target RNA scaffold. An asymmetrical internal loop of the present disclosure can be formed by 500 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 500 nucleotides on the engineered guide RNA side of the guide-target RNA scaffold. Thus, an asymmetrical internal loop can be a structural feature formed from latent structure provided by an engineered latent guide RNA.

As disclosed herein, a “base paired (bp) region” refers to a region of the guide-target RNA scaffold in which bases in the guide RNA are paired with opposing bases in the target RNA. Base paired regions can extend from one end or proximal to one end of the guide-target RNA scaffold to or proximal to the other end of the guide-target RNA scaffold. Base paired regions can extend between two structural features. Base paired regions can extend from one end or proximal to one end of the guide-target RNA scaffold to or proximal to a structural feature. Base paired regions can extend from a structural feature to the other end of the guide-target RNA scaffold. In some embodiments, a base paired region has from 1 bp to 100 bp, from 1 bp to 90 bp, from 1 bp to 80 bp, from 1 bp to 70 bp, from 1 bp to 60 bp, from 1 bp to 50 bp, from 1 bp to 45 bp, from 1 bp to 40 bp, from 1 bp to 35 bp, from 1 bp to 30 bp, from 1 bp to 25 bp, from 1 bp to 20 bp, from 1 bp to 15 bp, from 1 bp to 10 bp, from 1 bp to 5 bp, from 5 bp to 10 bp, from 5 bp to 20 bp, from 10 bp to 20 bp, from 10 bp to 50 bp, from 5 bp to 50 bp, at least 1 bp, at least 2 bp, at least 3 bp, at least 4 bp, at least 5 bp, at least 6 bp, at least 7 bp, at least 8 bp, at least 9 bp, at least 10 bp, at least 12 bp, at least 14 bp, at least 16 bp, at least 18 bp, at least 20 bp, at least 25 bp, at least 30 bp, at least 35 bp, at least 40 bp, at least 45 bp, at least 50 bp, at least 60 bp, at least 70 bp, at least 80 bp, at least 90 bp, at least 100 bp.

### Barbell Macro-Footprints

In some embodiments, an engineered guide RNA targeting LRRK2 can comprise a macro-footprint sequence such as a barbell macro-footprint. As disclosed herein, a barbell macro-footprint sequence, upon hybridization to a target RNA, produces a pair of internal loop structural features that improve one or more aspects of editing, as compared to an otherwise comparable guide RNA lacking the pair of internal loop structural features. In some instances, inclusion of a barbell macro-footprint sequence improves an amount of editing of an adenosine of interest (e.g., an on-target adenosine), relative to an amount of editing of on-target adenosine in a comparable guide RNA lacking the barbell macro-footprint sequence. In some instances, inclusion of a barbell macro-footprint sequence decreases an amount of editing of adenosines other than the adenosine of interest (e.g., decreases off-target adenosine), relative to an amount of off-target adenosine in a comparable guide RNA lacking the barbell macro-footprint sequence.

A macro-footprint sequence can be positioned such that it flanks a micro-footprint sequence. Further, while a macro-footprint sequence can flank a micro-footprint sequence, additional latent structures can be incorporated that flank either end of the macro-footprint as well. In some embodiments, such additional latent structures are included as part of the macro-footprint. In some embodiments, such additional latent structures are separate, distinct, or both separate and distinct from the macro-footprint.

In some embodiments, a macro-footprint sequence can comprise a barbell macro-footprint sequence comprising latent structures that, when manifested, produce a first internal loop and a second internal loop.

In some examples, a first internal loop is positioned “near the 5′ end of the guide-target RNA scaffold” and a second internal loop is positioned near the 3′ end of the guide-target RNA scaffold. The length of the dsRNA comprises a 5′ end and a 3′ end, where up to half of the length of the guide-target RNA scaffold at the 5′ end can be considered to be “near the 5′ end” while up to half of the length of the guide-target RNA scaffold at the 3′ end can be considered “near the 3′ end.” Non-limiting examples of the 5′ end can include about 50% or less of the total length of the dsRNA at the 5′ end, about 45%, about 40%, about 35%, about 30%, about 25%, about 20%, about 15%, about 10%, or about 5%. Non-limiting examples of the 3′ end can include about 50% or less of the total length of the dsRNA at the 3′ end about 45%, about 40%, about 35%, about 30%, about 25%, about 20%, about 15%, about 10%, or about 5%.

The engineered guide RNAs of the disclosure comprising a barbell macro-footprint sequence (that manifests as a first internal loop and a second internal loop) can improve RNA editing efficiency of a LRRK2 target RNA, increase the amount or percentage of RNA editing generally, as well as for on-target nucleotide editing, such as on-target adenosine. In some embodiments, the engineered guide RNAs of the disclosure comprising a first internal loop and a second internal loop can also facilitate a decrease in the amount of or reduce off-target nucleotide editing, such as off-target adenosine or unintended adenosine editing. The decrease or reduction in some examples can be of the number of off-target edits or the percentage of off-target edits.

In some embodiments, the first internal loop is positioned from about 7 bases away from the A/C mismatch to about 30 bases away from the A/C mismatch with respect to the base of the first internal loop that is most proximal to the A/C mismatch. In some embodiments, the first internal loop is positioned 10 bases away from the A/C mismatch with respect to the base of the first internal loop that is most proximal to the A/C mismatch. In some embodiments, the second internal loop is positioned from about 18 bases away from the A/C mismatch to about 34 bases away from the A/C mismatch with respect to the base of the second internal loop that is most proximal to the A/C mismatch. In some embodiments, the second internal loop is positioned 34 bases away from the A/C mismatch with respect to the base of the second internal loop that is most proximal to the A/C mismatch.

Each of the first and second internal loops of the barbell macro-footprint can independently be symmetrical or asymmetrical, where symmetry is determined by the number of bases or nucleotides of the engineered guide RNA and the number of bases or nucleotides of the target RNA, that together form each of the first and second internal loops.

As described herein, a double stranded RNA (dsRNA) substrate (a guide-target RNA scaffold) is formed upon hybridization of an engineered guide RNA of the present disclosure to an LRRK2 target RNA. An internal loop can be a symmetrical internal loop or an asymmetrical internal loop. A “symmetrical internal loop” is formed when the same number of nucleotides is present on each side of the internal loop. For example, a symmetrical internal loop in a guide-target RNA scaffold of the present disclosure can have the same number of nucleotides on the engineered guide RNA side and the target RNA side of the guide-target RNA scaffold. A symmetrical internal loop of the present disclosure can be formed by 5 nucleotides on the engineered guide RNA side of the guide-target RNA scaffold target and 5 nucleotides on the target RNA side of the guide-target RNA scaffold. A symmetrical internal loop of the present disclosure can be formed by 6 nucleotides on the engineered guide RNA side of the guide-target RNA scaffold target and 6 nucleotides on the target RNA side of the guide-target RNA scaffold. A symmetrical internal loop of the present disclosure can be formed by 7 nucleotides on the engineered guide RNA side of the guide-target RNA scaffold target and 7 nucleotides on the target RNA side of the guide-target RNA scaffold. A symmetrical internal loop of the present disclosure can be formed by 8 nucleotides on the engineered guide RNA side of the guide-target RNA scaffold target and 8 nucleotides on the target RNA side of the guide-target RNA scaffold. A symmetrical internal loop of the present disclosure can be formed by 9 nucleotides on the engineered guide RNA side of the guide-target RNA scaffold target and 9 nucleotides on the target RNA side of the guide-target RNA scaffold. A symmetrical internal loop of the present disclosure can be formed by 10 nucleotides on the engineered guide RNA side of the guide-target RNA scaffold target and 10 nucleotides on the target RNA side of the guide-target RNA scaffold. A symmetrical internal loop of the present disclosure can be formed by 15 nucleotides on the engineered polynucleotide side of the guide-target RNA scaffold target and 15 nucleotides on the target RNA side of the guide-target RNA scaffold. A symmetrical internal loop of the present disclosure can be formed by 20 nucleotides on the engineered polynucleotide side of the guide-target RNA scaffold target and 20 nucleotides on the target RNA side of the guide-target RNA scaffold. A symmetrical internal loop of the present disclosure can be formed by 30 nucleotides on the engineered polynucleotide side of the guide-target RNA scaffold target and 30 nucleotides on the target RNA side of the guide-target RNA scaffold. A symmetrical internal loop of the present disclosure can be formed by 40 nucleotides on the engineered polynucleotide side of the guide-target RNA scaffold target and 40 nucleotides on the target RNA side of the guide-target RNA scaffold. A symmetrical internal loop of the present disclosure can be formed by 50 nucleotides on the engineered polynucleotide side of the guide-target RNA scaffold target and 50 nucleotides on the target RNA side of the guide-target RNA scaffold. A symmetrical internal loop of the present disclosure can be formed by 60 nucleotides on the engineered polynucleotide side of the guide-target RNA scaffold target and 60 nucleotides on the target RNA side of the guide-target RNA scaffold. A symmetrical internal loop of the present disclosure can be formed by 70 nucleotides on the engineered polynucleotide side of the guide-target RNA scaffold target and 70 nucleotides on the target RNA side of the guide-target RNA scaffold. A symmetrical internal loop of the present disclosure can be formed by 80 nucleotides on the engineered polynucleotide side of the guide-target RNA scaffold target and 80 nucleotides on the target RNA side of the guide-target RNA scaffold. A symmetrical internal loop of the present disclosure can be formed by 90 nucleotides on the engineered polynucleotide side of the guide-target RNA scaffold target and 90 nucleotides on the target RNA side of the guide-target RNA scaffold. A symmetrical internal loop of the present disclosure can be formed by 100 nucleotides on the engineered polynucleotide side of the guide-target RNA scaffold target and 100 nucleotides on the target RNA side of the guide-target RNA scaffold. A symmetrical internal loop of the present disclosure can be formed by 110 nucleotides on the engineered polynucleotide side of the guide-target RNA scaffold target and 110 nucleotides on the target RNA side of the guide-target RNA scaffold. A symmetrical internal loop of the present disclosure can be formed by 120 nucleotides on the engineered polynucleotide side of the guide-target RNA scaffold target and 120 nucleotides on the target RNA side of the guide-target RNA scaffold. A symmetrical internal loop of the present disclosure can be formed by 130 nucleotides on the engineered polynucleotide side of the guide-target RNA scaffold target and 130 nucleotides on the target RNA side of the guide-target RNA scaffold. A symmetrical internal loop of the present disclosure can be formed by 140 nucleotides on the engineered polynucleotide side of the guide-target RNA scaffold target and 140 nucleotides on the target RNA side of the guide-target RNA scaffold. A symmetrical internal loop of the present disclosure can be formed by 150 nucleotides on the engineered polynucleotide side of the guide-target RNA scaffold target and 150 nucleotides on the target RNA side of the guide-target RNA scaffold. A symmetrical internal loop of the present disclosure can be formed by 200 nucleotides on the engineered polynucleotide side of the guide-target RNA scaffold target and 200 nucleotides on the target RNA side of the guide-target RNA scaffold. A symmetrical internal loop of the present disclosure can be formed by 250 nucleotides on the engineered polynucleotide side of the guide-target RNA scaffold target and 250 nucleotides on the target RNA side of the guide-target RNA scaffold. A symmetrical internal loop of the present disclosure can be formed by 300 nucleotides on the engineered polynucleotide side of the guide-target RNA scaffold target and 300 nucleotides on the target RNA side of the guide-target RNA scaffold. A symmetrical internal loop of the present disclosure can be formed by 350 nucleotides on the engineered polynucleotide side of the guide-target RNA scaffold target and 350 nucleotides on the target RNA side of the guide-target RNA scaffold. A symmetrical internal loop of the present disclosure can be formed by 400 nucleotides on the engineered polynucleotide side of the guide-target RNA scaffold target and 400 nucleotides on the target RNA side of the guide-target RNA scaffold. A symmetrical internal loop of the present disclosure can be formed by 450 nucleotides on the engineered polynucleotide side of the guide-target RNA scaffold target and 450 nucleotides on the target RNA side of the guide-target RNA scaffold. A symmetrical internal loop of the present disclosure can be formed by 500 nucleotides on the engineered polynucleotide side of the guide-target RNA scaffold target and 500 nucleotides on the target RNA side of the guide-target RNA scaffold. A symmetrical internal loop of the present disclosure can be formed by 600 nucleotides on the engineered polynucleotide side of the guide-target RNA scaffold target and 600 nucleotides on the target RNA side of the guide-target RNA scaffold. A symmetrical internal loop of the present disclosure can be formed by 700 nucleotides on the engineered polynucleotide side of the guide-target RNA scaffold target and 700 nucleotides on the target RNA side of the guide-target RNA scaffold. A symmetrical internal loop of the present disclosure can be formed by 800 nucleotides on the engineered polynucleotide side of the guide-target RNA scaffold target and 800 nucleotides on the target RNA side of the guide-target RNA scaffold. A symmetrical internal loop of the present disclosure can be formed by 900 nucleotides on the engineered polynucleotide side of the guide-target RNA scaffold target and 900 nucleotides on the target RNA side of the guide-target RNA scaffold. A symmetrical internal loop of the present disclosure can be formed by 1000 nucleotides on the engineered polynucleotide side of the guide-target RNA scaffold target and 1000 nucleotides on the target RNA side of the guide-target RNA scaffold. Thus, a symmetrical internal loop can be a structural feature formed from latent structure provided by an engineered latent guide RNA.

As described herein, a double stranded RNA (dsRNA) substrate (e.g., a guide-target RNA scaffold) is formed upon hybridization of an engineered guide RNA of the present disclosure to an LRRK2 target RNA. An internal loop can be a symmetrical internal loop or an asymmetrical internal loop. An “asymmetrical internal loop” is formed when a different number of nucleotides is present on each side of the internal loop. For example, an asymmetrical internal loop in a guide-target RNA scaffold of the present disclosure can have different numbers of nucleotides on the engineered guide RNA side and the target RNA side of the guide-target RNA scaffold.

An asymmetrical internal loop of the present disclosure can be formed by from 5 to 150 nucleotides on the engineered polynucleotide side of the guide-target RNA scaffold and from 5 to 150 nucleotides on the target RNA side of the guide-target RNA scaffold, wherein the number of nucleotides is the different on the engineered side of the guide-target RNA scaffold target than the number of nucleotides on the target RNA side of the guide-target RNA scaffold. An asymmetrical internal loop of the present disclosure can be formed by from 5 to 1000 nucleotides on the engineered polynucleotide side of the guide-target RNA scaffold and from 5 to 1000 nucleotides on the target RNA side of the guide-target RNA scaffold, wherein the number of nucleotides is the different on the engineered side of the guide-target RNA scaffold target than the number of nucleotides on the target RNA side of the guide-target RNA scaffold. An asymmetrical internal loop of the present disclosure can be formed by 5 nucleotides on the engineered guide RNA side of the guide-target RNA scaffold and 6 nucleotides on the target RNA side of the guide-target RNA scaffold. An asymmetrical internal loop of the present disclosure can be formed by 5 nucleotides on the target RNA side of the guide-target RNA scaffold and 6 nucleotides on the engineered guide RNA side of the guide-target RNA scaffold. An asymmetrical internal loop of the present disclosure can be formed by 5 nucleotides on the engineered guide RNA side of the guide-target RNA scaffold and 7 nucleotides on the target RNA side of the guide-target RNA scaffold. An asymmetrical internal loop of the present disclosure can be formed by 5 nucleotides on the target RNA side of the guide-target RNA scaffold and 7 nucleotides on the engineered guide RNA side of the guide-target RNA scaffold. An asymmetrical internal loop of the present disclosure can be formed by 5 nucleotides on the engineered guide RNA side of the guide-target RNA scaffold and 8 nucleotides internal loop the target RNA side of the guide-target RNA scaffold. An asymmetrical internal loop of the present disclosure can be formed by 5 nucleotides on the target RNA side of the guide-target RNA scaffold and 8 nucleotides on the engineered guide RNA side of the guide-target RNA scaffold. An asymmetrical internal loop of the present disclosure can be formed by 5 nucleotides on the engineered guide RNA side of the guide-target RNA scaffold and 9 nucleotides internal loop the target RNA side of the guide-target RNA scaffold. An asymmetrical internal loop of the present disclosure can be formed by 5 nucleotides on the target RNA side of the guide-target RNA scaffold and 9 nucleotides on the engineered guide RNA side of the guide-target RNA scaffold. An asymmetrical internal loop of the present disclosure can be formed by 5 nucleotides on the engineered guide RNA side of the guide-target RNA scaffold and 10 nucleotides internal loop the target RNA side of the guide-target RNA scaffold. An asymmetrical internal loop of the present disclosure can be formed by 5 nucleotides on the target RNA side of the guide-target RNA scaffold and 10 nucleotides on the engineered guide RNA side of the guide-target RNA scaffold. An asymmetrical internal loop of the present disclosure can be formed by 6 nucleotides on the engineered guide RNA side of the guide-target RNA scaffold and 7 nucleotides internal loop the target RNA side of the guide-target RNA scaffold. An asymmetrical internal loop of the present disclosure can be formed by 6 nucleotides on the target RNA side of the guide-target RNA scaffold and 7 nucleotides on the engineered guide RNA side of the guide-target RNA scaffold. An asymmetrical internal loop of the present disclosure can be formed by 6 nucleotides on the engineered guide RNA side of the guide-target RNA scaffold and 8 nucleotides internal loop the target RNA side of the guide-target RNA scaffold. An asymmetrical internal loop of the present disclosure can be formed by 6 nucleotides on the target RNA side of the guide-target RNA scaffold and 8 nucleotides on the engineered guide RNA side of the guide-target RNA scaffold. An asymmetrical internal loop of the present disclosure can be formed by 6 nucleotides on the engineered guide RNA side of the guide-target RNA scaffold and 9 nucleotides internal loop the target RNA side of the guide-target RNA scaffold. An asymmetrical internal loop of the present disclosure can be formed by 6 nucleotides on the target RNA side of the guide-target RNA scaffold and 9 nucleotides on the engineered guide RNA side of the guide-target RNA scaffold. An asymmetrical internal loop of the present disclosure can be formed by 6 nucleotides on the engineered guide RNA side of the guide-target RNA scaffold and 10 nucleotides internal loop the target RNA side of the guide-target RNA scaffold. An asymmetrical internal loop of the present disclosure can be formed by 6 nucleotides on the target RNA side of the guide-target RNA scaffold and 10 nucleotides on the engineered guide RNA side of the guide-target RNA scaffold. An asymmetrical internal loop of the present disclosure can be formed by 7 nucleotides on the engineered guide RNA side of the guide-target RNA scaffold and 8 nucleotides internal loop the target RNA side of the guide-target RNA scaffold. An asymmetrical internal loop of the present disclosure can be formed by 7 nucleotides on the target RNA side of the guide-target RNA scaffold and 8 nucleotides on the engineered guide RNA side of the guide-target RNA scaffold. An asymmetrical internal loop of the present disclosure can be formed by 7 nucleotides on the engineered guide RNA side of the guide-target RNA scaffold and 9 nucleotides internal loop the target RNA side of the guide-target RNA scaffold. An asymmetrical internal loop of the present disclosure can be formed by 7 nucleotides on the target RNA side of the guide-target RNA scaffold and 9 nucleotides on the engineered guide RNA side of the guide-target RNA scaffold. An asymmetrical internal loop of the present disclosure can be formed by 7 nucleotides on the engineered guide RNA side of the guide-target RNA scaffold and 10 nucleotides internal loop the target RNA side of the guide-target RNA scaffold. An asymmetrical internal loop of the present disclosure can be formed by 7 nucleotides on the target RNA side of the guide-target RNA scaffold and 10 nucleotides on the engineered guide RNA side of the guide-target RNA scaffold. An asymmetrical internal loop of the present disclosure can be formed by 8 nucleotides on the engineered guide RNA side of the guide-target RNA scaffold and 9 nucleotides internal loop the target RNA side of the guide-target RNA scaffold. An asymmetrical internal loop of the present disclosure can be formed by 8 nucleotides on the target RNA side of the guide-target RNA scaffold and 9 nucleotides on the engineered guide RNA side of the guide-target RNA scaffold. An asymmetrical internal loop of the present disclosure can be formed by 8 nucleotides on the engineered guide RNA side of the guide-target RNA scaffold and 10 nucleotides internal loop the target RNA side of the guide-target RNA scaffold. An asymmetrical internal loop of the present disclosure can be formed by 8 nucleotides on the target RNA side of the guide-target RNA scaffold and 10 nucleotides on the engineered guide RNA side of the guide-target RNA scaffold. An asymmetrical internal loop of the present disclosure can be formed by 9 nucleotides on the engineered guide RNA side of the guide-target RNA scaffold and 10 nucleotides internal loop the target RNA side of the guide-target RNA scaffold. An asymmetrical internal loop of the present disclosure can be formed by 9 nucleotides on the target RNA side of the guide-target RNA scaffold and 10 nucleotides on the engineered guide RNA side of the guide-target RNA scaffold. An asymmetrical internal loop of the present disclosure can be formed by 5 nucleotides on the target RNA side of the guide-target RNA scaffold and 50 nucleotides on the engineered polynucleotide side of the guide-target RNA scaffold. An asymmetrical internal loop of the present disclosure can be formed by 5 nucleotides on the target RNA side of the guide-target RNA scaffold and 100 nucleotides on the engineered polynucleotide side of the guide-target RNA scaffold. An asymmetrical internal loop of the present disclosure can be formed by 5 nucleotides on the target RNA side of the guide-target RNA scaffold and 150 nucleotides on the engineered polynucleotide side of the guide-target RNA scaffold. An asymmetrical internal loop of the present disclosure can be formed by 5 nucleotides on the target RNA side of the guide-target RNA scaffold and 200 nucleotides on the engineered polynucleotide side of the guide-target RNA scaffold. An asymmetrical internal loop of the present disclosure can be formed by 5 nucleotides on the target RNA side of the guide-target RNA scaffold and 300 nucleotides on the engineered polynucleotide side of the guide-target RNA scaffold. An asymmetrical internal loop of the present disclosure can be formed by 5 nucleotides on the target RNA side of the guide-target RNA scaffold and 400 nucleotides on the engineered polynucleotide side of the guide-target RNA scaffold. An asymmetrical internal loop of the present disclosure can be formed by 5 nucleotides on the target RNA side of the guide-target RNA scaffold and 500 nucleotides on the engineered polynucleotide side of the guide-target RNA scaffold. An asymmetrical internal loop of the present disclosure can be formed by 5 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 5 nucleotides on the engineered polynucleotide side of the guide-target RNA scaffold. An asymmetrical internal loop of the present disclosure can be formed by 500 nucleotides on the target RNA side of the guide-target RNA scaffold and 5 nucleotides on the engineered polynucleotide side of the guide-target RNA scaffold. An asymmetrical internal loop of the present disclosure can be formed by 400 nucleotides on the target RNA side of the guide-target RNA scaffold and 5 nucleotides on the engineered polynucleotide side of the guide-target RNA scaffold. An asymmetrical internal loop of the present disclosure can be formed by 300 nucleotides on the target RNA side of the guide-target RNA scaffold and 5 nucleotides on the engineered polynucleotide side of the guide-target RNA scaffold. An asymmetrical internal loop of the present disclosure can be formed by 200 nucleotides on the target RNA side of the guide-target RNA scaffold and 5 nucleotides on the engineered polynucleotide side of the guide-target RNA scaffold. An asymmetrical internal loop of the present disclosure can be formed by 150 nucleotides on the target RNA side of the guide-target RNA scaffold and 5 nucleotides on the engineered polynucleotide side of the guide-target RNA scaffold. An asymmetrical internal loop of the present disclosure can be formed by 100 nucleotides on the target RNA side of the guide-target RNA scaffold and 5 nucleotides on the engineered polynucleotide side of the guide-target RNA scaffold. An asymmetrical internal loop of the present disclosure can be formed by 50 nucleotides on the target RNA side of the guide-target RNA scaffold and 5 nucleotides on the engineered polynucleotide side of the guide-target RNA scaffold. An asymmetrical internal loop of the present disclosure can be formed by 50 nucleotides on the target RNA side of the guide-target RNA scaffold and 100 nucleotides on the engineered polynucleotide side of the guide-target RNA scaffold. An asymmetrical internal loop of the present disclosure can be formed by 50 nucleotides on the target RNA side of the guide-target RNA scaffold and 150 nucleotides on the engineered polynucleotide side of the guide-target RNA scaffold. An asymmetrical internal loop of the present disclosure can be formed by 50 nucleotides on the target RNA side of the guide-target RNA scaffold and 200 nucleotides on the engineered polynucleotide side of the guide-target RNA scaffold. An asymmetrical internal loop of the present disclosure can be formed by 50 nucleotides on the target RNA side of the guide-target RNA scaffold and 300 nucleotides on the engineered polynucleotide side of the guide-target RNA scaffold. An asymmetrical internal loop of the present disclosure can be formed by 50 nucleotides on the target RNA side of the guide-target RNA scaffold and 400 nucleotides on the engineered polynucleotide side of the guide-target RNA scaffold. An asymmetrical internal loop of the present disclosure can be formed by 50 nucleotides on the target RNA side of the guide-target RNA scaffold and 500 nucleotides on the engineered polynucleotide side of the guide-target RNA scaffold. An asymmetrical internal loop of the present disclosure can be formed by 50 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 50 nucleotides on the engineered polynucleotide side of the guide-target RNA scaffold. An asymmetrical internal loop of the present disclosure can be formed by 500 nucleotides on the target RNA side of the guide-target RNA scaffold and 50 nucleotides on the engineered polynucleotide side of the guide-target RNA scaffold. An asymmetrical internal loop of the present disclosure can be formed by 400 nucleotides on the target RNA side of the guide-target RNA scaffold and 50 nucleotides on the engineered polynucleotide side of the guide-target RNA scaffold. An asymmetrical internal loop of the present disclosure can be formed by 300 nucleotides on the target RNA side of the guide-target RNA scaffold and 50 nucleotides on the engineered polynucleotide side of the guide-target RNA scaffold. An asymmetrical internal loop of the present disclosure can be formed by 200 nucleotides on the target RNA side of the guide-target RNA scaffold and 50 nucleotides on the engineered polynucleotide side of the guide-target RNA scaffold. An asymmetrical internal loop of the present disclosure can be formed by 150 nucleotides on the target RNA side of the guide-target RNA scaffold and 50 nucleotides on the engineered polynucleotide side of the guide-target RNA scaffold. An asymmetrical internal loop of the present disclosure can be formed by 100 nucleotides on the target RNA side of the guide-target RNA scaffold and 50 nucleotides on the engineered polynucleotide side of the guide-target RNA scaffold. An asymmetrical internal loop of the present disclosure can be formed by 100 nucleotides on the target RNA side of the guide-target RNA scaffold and 150 nucleotides on the engineered polynucleotide side of the guide-target RNA scaffold. An asymmetrical internal loop of the present disclosure can be formed by 100 nucleotides on the target RNA side of the guide-target RNA scaffold and 200 nucleotides on the engineered polynucleotide side of the guide-target RNA scaffold. An asymmetrical internal loop of the present disclosure can be formed by 100 nucleotides on the target RNA side of the guide-target RNA scaffold and 300 nucleotides on the engineered polynucleotide side of the guide-target RNA scaffold. An asymmetrical internal loop of the present disclosure can be formed by 100 nucleotides on the target RNA side of the guide-target RNA scaffold and 400 nucleotides on the engineered polynucleotide side of the guide-target RNA scaffold. An asymmetrical internal loop of the present disclosure can be formed by 100 nucleotides on the target RNA side of the guide-target RNA scaffold and 500 nucleotides on the engineered polynucleotide side of the guide-target RNA scaffold. An asymmetrical internal loop of the present disclosure can be formed by 100 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 100 nucleotides on the engineered polynucleotide side of the guide-target RNA scaffold. An asymmetrical internal loop of the present disclosure can be formed by 500 nucleotides on the target RNA side of the guide-target RNA scaffold and 100 nucleotides on the engineered polynucleotide side of the guide-target RNA scaffold. An asymmetrical internal loop of the present disclosure can be formed by 400 nucleotides on the target RNA side of the guide-target RNA scaffold and 100 nucleotides on the engineered polynucleotide side of the guide-target RNA scaffold. An asymmetrical internal loop of the present disclosure can be formed by 300 nucleotides on the target RNA side of the guide-target RNA scaffold and 100 nucleotides on the engineered polynucleotide side of the guide-target RNA scaffold. An asymmetrical internal loop of the present disclosure can be formed by 200 nucleotides on the target RNA side of the guide-target RNA scaffold and 100 nucleotides on the engineered polynucleotide side of the guide-target RNA scaffold. An asymmetrical internal loop of the present disclosure can be formed by 150 nucleotides on the target RNA side of the guide-target RNA scaffold and 100 nucleotides on the engineered polynucleotide side of the guide-target RNA scaffold. An asymmetrical internal loop of the present disclosure can be formed by 150 nucleotides on the target RNA side of the guide-target RNA scaffold and 200 nucleotides on the engineered polynucleotide side of the guide-target RNA scaffold. An asymmetrical internal loop of the present disclosure can be formed by 150 nucleotides on the target RNA side of the guide-target RNA scaffold and 300 nucleotides on the engineered polynucleotide side of the guide-target RNA scaffold. An asymmetrical internal loop of the present disclosure can be formed by 150 nucleotides on the target RNA side of the guide-target RNA scaffold and 400 nucleotides on the engineered polynucleotide side of the guide-target RNA scaffold. An asymmetrical internal loop of the present disclosure can be formed by 150 nucleotides on the target RNA side of the guide-target RNA scaffold and 500 nucleotides on the engineered polynucleotide side of the guide-target RNA scaffold. An asymmetrical internal loop of the present disclosure can be formed by 150 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 150 nucleotides on the engineered polynucleotide side of the guide-target RNA scaffold. An asymmetrical internal loop of the present disclosure can be formed by 500 nucleotides on the target RNA side of the guide-target RNA scaffold and 5 nucleotides on the engineered polynucleotide side of the guide-target RNA scaffold. An asymmetrical internal loop of the present disclosure can be formed by 400 nucleotides on the target RNA side of the guide-target RNA scaffold and 150 nucleotides on the engineered polynucleotide side of the guide-target RNA scaffold. An asymmetrical internal loop of the present disclosure can be formed by 300 nucleotides on the target RNA side of the guide-target RNA scaffold and 150 nucleotides on the engineered polynucleotide side of the guide-target RNA scaffold. An asymmetrical internal loop of the present disclosure can be formed by 200 nucleotides on the target RNA side of the guide-target RNA scaffold and 300 nucleotides on the engineered polynucleotide side of the guide-target RNA scaffold. An asymmetrical internal loop of the present disclosure can be formed by 200 nucleotides on the target RNA side of the guide-target RNA scaffold and 400 nucleotides on the engineered polynucleotide side of the guide-target RNA scaffold. An asymmetrical internal loop of the present disclosure can be formed by 200 nucleotides on the target RNA side of the guide-target RNA scaffold and 500 nucleotides on the engineered polynucleotide side of the guide-target RNA scaffold. An asymmetrical internal loop of the present disclosure can be formed by 200 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 200 nucleotides on the engineered polynucleotide side of the guide-target RNA scaffold. An asymmetrical internal loop of the present disclosure can be formed by 500 nucleotides on the target RNA side of the guide-target RNA scaffold and 200 nucleotides on the engineered polynucleotide side of the guide-target RNA scaffold. An asymmetrical internal loop of the present disclosure can be formed by 400 nucleotides on the target RNA side of the guide-target RNA scaffold and 200 nucleotides on the engineered polynucleotide side of the guide-target RNA scaffold. An asymmetrical internal loop of the present disclosure can be formed by 300 nucleotides on the target RNA side of the guide-target RNA scaffold and 200 nucleotides on the engineered polynucleotide side of the guide-target RNA scaffold. An asymmetrical internal loop of the present disclosure can be formed by 300 nucleotides on the target RNA side of the guide-target RNA scaffold and 400 nucleotides on the engineered polynucleotide side of the guide-target RNA scaffold. An asymmetrical internal loop of the present disclosure can be formed by 300 nucleotides on the target RNA side of the guide-target RNA scaffold and 500 nucleotides on the engineered polynucleotide side of the guide-target RNA scaffold. An asymmetrical internal loop of the present disclosure can be formed by 300 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 300 nucleotides on the engineered polynucleotide side of the guide-target RNA scaffold. An asymmetrical internal loop of the present disclosure can be formed by 500 nucleotides on the target RNA side of the guide-target RNA scaffold and 300 nucleotides on the engineered polynucleotide side of the guide-target RNA scaffold. An asymmetrical internal loop of the present disclosure can be formed by 400 nucleotides on the target RNA side of the guide-target RNA scaffold and 300 nucleotides on the engineered polynucleotide side of the guide-target RNA scaffold. An asymmetrical internal loop of the present disclosure can be formed by 400 nucleotides on the target RNA side of the guide-target RNA scaffold and 500 nucleotides on the engineered polynucleotide side of the guide-target RNA scaffold. An asymmetrical internal loop of the present disclosure can be formed by 400 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 400 nucleotides on the engineered polynucleotide side of the guide-target RNA scaffold. An asymmetrical internal loop of the present disclosure can be formed by 500 nucleotides on the target RNA side of the guide-target RNA scaffold and 400 nucleotides on the engineered polynucleotide side of the guide-target RNA scaffold. An asymmetrical internal loop of the present disclosure can be formed by 500 nucleotides on the target RNA side of the guide-target RNA scaffold and 1000 nucleotides on the engineered polynucleotide side of the guide-target RNA scaffold. An asymmetrical internal loop of the present disclosure can be formed by 1000 nucleotides on the target RNA side of the guide-target RNA scaffold and 500 nucleotides on the engineered polynucleotide side of the guide-target RNA scaffold. Thus, an asymmetrical internal loop can be a structural feature formed from latent structure provided by an engineered latent guide RNA.

In some embodiments, a first internal loop or a second internal loop can independently comprise a number of bases of at least about 5 bases or greater (e.g., 6, 7, 8, 9, 10, 11, 12, 13, 14, 15, 16, 17, 18, 19, 20, 30, 40, 50, 60, 70, 80, 90, 100, 110, 120, 130, 140, 150); about 150 bases or fewer (e.g., 145, 135, 125, 115, 95, 85, 75, 65, 55, 45, 35, 25, 19, 18, 17, 16, 15, 14, 13, 12, 11, 10, 9, 8, 7, 6, 5); or at least about 5 bases to at least about 150 bases (e.g., 5-150, 6-145, 7-140, 8-135, 9-130, 10-125, 11-120, 12-115, 13-110, 14-105, 15-100, 16-95, 17-90, 18-85, 19-80, 20-75, 21-70, 22-65, 23-60, 24-55, 25-50) of the engineered guide RNA and a number of bases of at least about 5 bases or greater (e.g., 6, 7, 8, 9, 10, 11, 12, 13, 14, 15, 16, 17, 18, 19, 20, 30, 40, 50, 60, 70, 80, 90, 100, 110, 120, 130, 140, 150); about 150 bases or fewer (e.g., 145, 135, 125, 115, 95, 85, 75, 65, 55, 45, 35, 25, 19, 18, 17, 16, 15, 14, 13, 12, 11, 10, 9, 8, 7, 6, 5); or at least about 5 bases to at least about 150 bases (e.g., 5-150, 6-145, 7-140, 8-135, 9-130, 10-125, 11-120, 12-115, 13-110, 14-105, 15-100, 16-95, 17-90, 18-85, 19-80, 20-75, 21-70, 22-65, 23-60, 24-55, 25-50) of the target RNA.

In some embodiments, an engineered guide RNA comprising a barbell macro-footprint (e.g., a latent structure that manifests as a first internal loop and a second internal loop) comprises a cytosine in a micro-footprint sequence in between the macro-footprint sequence that, when the engineered guide RNA is hybridized to the LRRK2 target RNA, is present in the guide-target RNA scaffold opposite an adenosine that is edited by the RNA editing entity (e.g., an on-target adenosine). In such embodiments, the cytosine of the micro-footprint is comprised in an A/C mismatch with the on-target adenosine of the target RNA in the guide-target RNA scaffold.

A first internal loop and a second internal loop of the barbell macro-footprint can be positioned a certain distance from the A/C mismatch, with respect to the base of the first internal loop and the base of the second internal loop that is the most proximal to the A/C mismatch. In some embodiments, the first internal loop and the second internal loop can be positioned the same number of bases from the A/C mismatch, with respect to the base of the first internal loop and the base of the second internal loop that is the most proximal to the A/C mismatch. In some embodiments, the first internal loop and the second internal loop can be positioned a different number of bases from the A/C mismatch, with respect to the base of the first internal loop and the base of the second internal loop that is the most proximal to the A/C mismatch.

In some embodiments, the first internal loop of the barbell or the second internal loop of the barbell can be positioned at least about 5 bases (e.g., 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 bases) away from the A/C mismatch with respect to the base of the first internal loop or the second internal loop that is the most proximal to the A/C mismatch. In some embodiments, the first internal loop of the barbell or the second internal loop of the barbell can be positioned at most about 50 bases away from the A/C mismatch (e.g., 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5) with respect to the base of the first internal loop or the second internal loop that is the most proximal to the A/C mismatch.

In some embodiments, the first internal loop can be positioned from about 5 bases away from the A/C mismatch to about 15 bases away from the A/C mismatch (e.g., 6-14, 7-13, 8-12, 9-11) with respect to the base of the first internal loop that is most proximal to the A/C mismatch. In some examples, the first internal loop can be positioned from about 9 bases away from the A/C mismatch to about 15 bases away from the A/C mismatch (e.g., 10-14, 11-13) with respect to the base of the first internal loop that is the most proximal to the A/C mismatch.

In some embodiments, the second internal loop can be positioned from about 12 bases away from the A/C mismatch to about 40 bases away from the A/C mismatch (e.g., 13-39, 14-38, 15-37, 16-36, 17-35, 18-34, 19-33, 20-32, 21-31, 22-30, 23-29, 24-28, 25-27) with respect to the base of the second internal loop that is the most proximal to the A/C mismatch. In some embodiments, the second internal loop can be positioned from about 20 bases away from the A/C mismatch to about 33 bases away from the A/C mismatch with respect to the base of the second internal loop that is most proximal to the A/C mismatch.

TABLE 1
EXEMPLARY GUIDE RNAS THAT TARGET LRRK2 MRNA

| SEQ | | | |
|---|---|---|---|
| ID NO | ID | Sequence | Structural Features (target/guide) |
| SEQ | LRRK2_bnPCR_ID9 | CCCTGGTGTGCCC | 1/1 U/G wobble base pair at −3 position; |
| ID | 4725_count351_ | TCTGATGTTTTTA | 1/1 A/C mismatch at 0 position; |
| NO: 2 | NoLoops_gID_02513 | TCCCCATTCTACA | 2_2/2 symmetric bulge at position +2 (CA-AU); |
| | | GCGGTACTGAGC | 1/1 U-G wobble base pair at +15 |
| | | AAATCCGTGGTC | |
| | | AGCAATCTTTGCA | |
| | | ATGATGGCAGCA | |
| | | TTGGGATACAGT | |
| | | GTGAAGAGCAGC | |
| | | A | |
| SEQ | LRRK2_bnPCR_ID9 | CCCTGGTGTGCCC | 6/6_symmetric internal loop at −14_position |
| ID | 4725_count351_- | TCTGATGTTTTTT | (UGCAAA-AAACGU); |
| NO: 3 | 14_26_gID_08570 | AGGGGATTCTAC | 1/1_U/G wobble base pair at −3 position; |
| | | AGCGGTACTGAG | 1/1 A/C mismatch at 0 position; |
| | | CAAATCCGTGGT | 2/2 CA/AU symmetric bulge at +2_position; |
| | | CAGCAATCAAAC | 1/1_U/G wobble at +15 position; |
| | | GTATGATGGCAG | 6/6 symmetric internal loop at +26 position (GGGGAU- |
| | | CATTGGGATACA | UAGGGG) |
| | | GTGTGAAGAGCA | |
| | | GCA | |
| SEQ | LRRK2_bnPCR_ID7 | CCCTGGTGTGCCC | 1/1 C/C mismatch at −4 position; |
| ID | 9791_count610_ | TCTGATGTTTTTA | 1/1 (U/G) wobble base pair at −3 position; |
| NO: 4 | NoLoops_gID_06724 | TCCCCATTCTACA | 1/1 A/C mismatch at 0 position; |
| | | TCTGTAGTGAGC | 1/1 C/U mismatch at +2 position; 1/1 G/G mismatch at |
| | | AATTCCGTGCTCA | +11 position; 1/1 U/U mismatch at +15 position; 1/1 |
| | | GCAATCTTTGCAA | C/U mismatch at +17 position |
| | | TGATGGCAGCAT | |
| | | TGGGATACAGTG | |
| | | TGAAGAGCAGCA | |
| SEQ | LRRK2_bnPCR_ID7 | CCCTGGTGTGCCC | 6/6 symmetric internal loop at −14 position (UGCAAA- |
| ID | 9791_count610_- | TCTGATGTTTTTT | AAACGU); |
| NO: 5 | 14_26_gID_09469 | AGGGGATTCTAC | 1/1 C/C mismatch at −4 position; |
| | | ATCTGTAGTGAG | 1/1 U/G wobble base pair at −3 position; |
| | | CAATTCCGTGCTC | 1/1 A/C mismatch at 0 position; |
| | | AGCAATCAAACG | 1/1 C/U mismatch at +2 position; |
| | | TATGATGGCAGC | 1/1 G/G mismatch at +11 position; |
| | | ATTGGGATACAG | 1/1 U/U mismatch +15 position; |
| | | TGTGAAGAGCAG | 1/1 C/U mismatch at +17 position; |
| | | CA | 6/6 symmetric internal loop at +26 position (GGGGAU- |
| | | | UAGGGG) |
| SEQ | LRRK2_bnPCR_ID6 | CCCTGGTGTGCCC | 1/1 C/C mismatch at −4 position; |
| ID | 6010_count1326 | TCTGATGTTTTTA | 1/1 A/C mismatch at 0 position; |
| NO: 6 | NoLoops_gID_092 | TCCCCATTCTACA | 1/1 C/C mismatch at +2 position; |
| | 47 | CCTGGACTGAGA | 1/1 G/A mismatch at +6 position; |
| | | AATCCCGTACTCA | 3/3 symmetric bulge at +13 position (ACU-UGG); |
| | | GCAATCTTTGCAA | 1/1 C/C mismatch at +17 position |
| | | TGATGGCAGCAT | |
| | | TGGGATACAGTG | |
| | | TGAAGAGCAGCA | |
| SEQ | LRRK2_bnPCR ID6 | CCCTGGTGTGCCC | 6/6 symmetric internal loop at −14 position (UGCAAA- |
| ID | 6010_count1326_- | TCTGATGTTTTTT | AAACGU); |
| NO: 7 | 14_26_gID_11327 | AGGGGATTCTAC | 1/1 C/C mismatch at −4 position; |
| | | ACCTGGACTGAG | 1/1 A/C mismatch at 0 position; |
| | | AAATCCCGTACTC | 1/1 C/C mismatch at +2 position; |
| | | AGCAATCAAACG | 1/1 G/A mismatch at +6 position; |
| | | TATGATGGCAGC | 3/3 symmetric bulge at +13 position (ACU-UGG); |
| | | ATTGGGATACAG | 1/1 C/C mismatch at +17 position; |
| | | TGTGAAGAGCAG | 6/6_symmetric internal loop at +26 position (GGGGAU- |
| | | CA | UAGGGG) |
| SEQ | LRRK2_bnPCR_ID5 | CCCTGGTGTGCCC | 1/1 U/G wobble base pair at −3 position; |
| ID | 0437_count708_ | TCTGATGTTTTTA | 1/1 A/C mismatch at 0 position; |
| NO: 8 | NoLoops_gID_11520 | TCCCCATTCTACA | 1/1 U/G wobble base pair at +4 position; |
| | | GCAGTACGGTGC | 1/1 U/U mismatch at +8 position; |
| | | AGTGCCGTGGTC | 1/1 A/G mismatch at +10 position |
| | | AGCAATCTTTGCA | |
| | | ATGATGGCAGCA | |
| | | TTGGGATACAGT | |
| | | GTGAAGAGCAGC | |
| | | A | |
| SEQ | LRRK2_bnPCR ID5 | CCCTGGTGTGCCC | 6/6 symmetric internal loop at −14 position (UGCAAA- |
| ID | 0437_count708_- | TCTGATGTTTTTT | AAACGU); |
| NO: 9 | 14_26_gID_09857 | AGGGGATTCTAC | 1/1 U/G wobble at −3 position; |
| | | AGCAGTACGGTG | 1/1 A/C mismatch at 0 position; |
| | | CAGTGCCGTGGT | 1/1 U/G wobble base pair at +4 position; |
| | | CAGCAATCAAAC | 1/1 U/U mismatch at +8 position; |
| | | GTATGATGGCAG | 1/1 A/G mismatch at +10 position; |
| | | CATTGGGATACA | 6/6 symmetric internal loop at +26 position (GGGGAU- |
| | | GTGTGAAGAGCA | UAGGGG) |
| | | GCA | |
| SEQ | LRRK2_bnPCR_ID4 | CCCTGGTGTGCCC | 1/1 U/G wobble base pair at −3 position; |
| ID | 1799_count730_ | TCTGATGTTTTTA | 1/1 A/C mismatch at 0 position; |
| NO: | NoLoops_gID_04947 | TCCCCCTTCTACA | 2/2 symmetric bulge at +4 position (UU-UU); |
| 10 | | GCAGTAGTGAGT | 1/1 G/U wobble base pair at +6 position; |
| | | TTTGCCGTGGTCA | 1/1 G/G mismatch at +11 position; |
| | | GCAATCTTTGCAA | 1/1 U/C mismatch at +25 position |
| | | TGATGGCAGCAT | |
| | | TGGGATACAGTG | |
| | | TGAAGAGCAGCA | |
| SEQ | LRRK2_bnPCR_ID4 | CCCTGGTGTGCCC | 6/6 symmetric internal loop at −14_position (UGCAAA- |
| ID | 1799_count730_- | TCTGATGTTTTTT | AAACGU); |
| NO: | 14_26_gID_07755 | AGGGGCTTCTAC | 1/1 U/G wobble base pair at −3 position; |
| 11 | | AGCAGTAGTGAG | 1/1 A/C mismatch at 0 position; |
| | | TTTTGCCGTGGTC | 2/2 symmetric bulge at +4 position (UU-UU); |
| | | AGCAATCAAACG | 1/1 G/U wobble base pair at +6 position; |
| | | TATGATGGCAGC | 1/1 G/G mismatch at +11 position; |
| | | ATTGGGATACAG | 7/7 symmetric internal loop at +25 position |
| | | TGTGAAGAGCAG | (UGGGGAU-UAGGGGC) |
| | | CA | |
| SEQ | LRRK2_bnPCR ID3 | CCCTGGTGTGCCC | 1/1 C/A mismatch at −4 position; |
| ID | 5277_count295_ | TCTGATGTTTTTA | 1/1 U/G wobble base pair at −3 position; |
| NO: | NoLoops_gID_06840 | TCCCCATTCTACA | 1/1 A/C mismatch at 0 position; |
| 12 | | GCAGTCCTGTAC | 1/1 U/G wobble base pair at +4 position; |
| | | AGTGCCGTGATC | 2/2 symmetric bulge at +7 position (CU-UA); |
| | | AGCAATCTTTGCA | 1/1 U/C mismatch at +12 position |
| | | ATGATGGCAGCA | |
| | | TTGGGATACAGT | |
| | | GTGAAGAGCAGC | |
| | | A | |
| SEQ | LRRK2_bnPCR_ID3 | CCCTGGTGTGCCC | 6/6 symmetric internal loop at −14 position (UGCAAA- |
| ID | 5277_count295_- | TCTGATGTTTTTT | AAACGU); |
| NO: | 14_26_gID_09359 | AGGGGATTCTAC | 1/1 C/A mismatch at −4 position; |
| 13 | | AGCAGTCCTGTA | 1/1 U/G wobble base pair at −3 position; |
| | | CAGTGCCGTGAT | 1/1 A/C mismatch at 0 position; |
| | | CAGCAATCAAAC | 1/1 U/G wobble base pair at +4 position; |
| | | GTATGATGGCAG | 2/2 symmetric bulge at +7 position (CU-UA); |
| | | CATTGGGATACA | 1/1 U/C mismatch at +12 position; |
| | | GTGTGAAGAGCA | 6/6 symmetric internal loop at +26 position (GGGGAU- |
| | | GCA | UAGGGG |
| SEQ | LRRK2_bnPCR_ID3 | CCCTGGTGTGCCC | 6/6 symmetric internal loop at −10 position (AAGAUU- |
| ID | 4601_count2108_- | TCTGATGTTTTTA | CUAGGC); |
| NO: | 10_16_gID_01444 | TCCCCATTCGCCT | 1/1 G/U wobble base pair at −6 position; |
| 14 | | GAGGTACTGACC | 1/1 A/C mismatch at 0 position; |
| | | AATCCCGTAGTTA | 1/1 C/C mismatch at +2 position; |
| | | GCCTAGGCTGCA | 1/1 C/C mismatch at +7 position; |
| | | ATGATGGCAGCA | 1/1 U/G wobble base pair at +15 position; |
| | | TTGGGATACAGT | 6/6 symmetric internal loop at +16 position (GCUGUA- |
| | | GTGAAGAGCAGC | GCCUGA) |
| | | A | |
| SEQ | LRRK2_bnPCR_ID3 | CCCTGGTGTGCCC | 6/6 symmetric internal loop at −22 position (GCCAUC- |
| ID | 3405_count860_- | TCTGATGTTTTTC | AACCUG); |
| NO: | 22_26_gID_00318 | TACAGATTCTACA | 1/1 C/C mismatch at −8 position; |
| 15 | | GCAGTACAGAGG | 1/1 U/G wobble base pair at −3 position; |
| | | ACTGCCGAGGTC | 1/1 A/A mismatch at −2 position; |
| | | ACCAATCTTTGCA | 1/1 A/C mismatch at 0 position; |
| | | ATAACCTGAGCA | 1/1 U/C mismatch at +4 position; |
| | | TTGGGATACAGT | 1/1 G/G mismatch at +6 position; |
| | | GTGAAGAGCAGC | 1/1 A/A mismatch at +10 position; |
| | | A | 2/2 symmetric bulge at +26 position (GG-AG); |
| | | | 3/3 symmetric bulge at +29 position (GAU-CUA) |
| SEQ | LRRK2_bnPCR_ID3 | CCCTGGTGTGCCC | 6/6 symmetric internal loop at −20 position (CAUCAU- |
| ID | 3405_count860_- | TCTGATGTTTTTT | UACUAC); |
| NO: | 20_26_gID_09192 | AGGGGATTCTAC | 1/1 C/C mismatch at −8 position; |
| 16 | | AGCAGTACAGAG | 1/1 U/G wobble base pair at −3 position; |
| | | GACTGCCGAGGT | 1/1 A/A mismatch at −2 position; |
| | | CACCAATCTTTGC | 1/1 A/C mismatch at 0 position; |
| | | ATACTACGCAGC | 1/1 U/C mismatch at +4 position; 1/1 G/G mismatch at |
| | | ATTGGGATACAG | +6 position; |
| | | TGTGAAGAGCAG | 1/1 A/A mismatch at +10 position; 6/6 symmetric |
| | | CA | internal loop at +26 position (GGGGAU-UAGGGG) |
| SEQ | LRRK2_bnPCR ID3 | CCCTGGTGTGCCC | 6/6 symmetric internal loop at −16 position (AUUGCA- |
| ID | 3405_count860_- | TCTGATGTTTTTC | GUCUAG); |
| NO: | 16_26_gID_11898 | GGGAGATTCTAC | 1/1 C/C mismatch at −8 position; |
| 17 | | AGCAGTACAGAG | 1/1 U/G wobble at −3 position; |
| | | GACTGCCGAGGT | 1/1 A/A mismatch at −2 position; |
| | | CACCAATCTTGTC | 1/1 A/C mismatch at 0 position; |
| | | TAGGATGGCAGC | 1/1 U/C mismatch at +4 position; |
| | | ATTGGGATACAG | 1/1 G/G mismatch at +6 position; |
| | | TGTGAAGAGCAG | 1/1 A/A mismatch at +10 position; 6/6 symmetric |
| | | CA | internal loop at +26 position (GGGGAU-CGGGAG) |
| SEQ | LRRK2_bnPCR_ID3 | CCCTGGTGTGCCC | 6/6 symmetric internal loop at −12 position (CAAAGA- |
| ID | 3405_count860_- | TCTGATGTATGTT | CGCUAA); |
| NO: | 12_30_gID_05698 | CCCCCATTCTACA | 1/1 C/C mismatch at −8 position; |
| 18 | | GCAGTACAGAGG | 1/1 U/G wobble base pair at −3 position; |
| | | ACTGCCGAGGTC | 1/1 A/A mismatch at −2 position; |
| | | ACCAACGCTAAC | 1/1 A/C mismatch at 0 position; |
| | | AATGATGGCAGC | 1/1 U/C mismatch at +4 position; |
| | | ATTGGGATACAG | 1/1 G/G mismatch at +6 position; |
| | | TGTGAAGAGCAG | 1/1 A/A mismatch at +10 position; |
| | | CA | 4/4 symmetric bulge at +30 position (AUAA-GUUC); |
| | | | 1/1 A/A mismatch at +35 position |
| SEQ | LRRK2_bnPCR_ID3 | CCCTGGTGTGCCC | 6/6 symmetric internal loop at −8 position (GAUUGC- |
| ID | 3405_count860_- | TCTGATGTTTTTA | CUUUGA); |
| NO: | 8_24_gID_05952 | TGGAAAGTCTAC | 1/1 U/G wobble base pair at −3 position; |
| 19 | | AGCAGTACAGAG | 1/1 A/A mismatch at −2 position; |
| | | GACTGCCGAGGT | 1/1 A/C mismatch at 0 position; |
| | | CACTTTGATTTGC | 1/1 U/C mismatch at +4 position; |
| | | AATGATGGCAGC | 1/1 G/G mismatch at +6 position; |
| | | ATTGGGATACAG | 1/1 A/A mismatch at +10 position; |
| | | TGTGAAGAGCAG | 6/6 symmetric internal loop at +24 position (AUGGGG- |
| | | CA | GGAAAG) |
| SEQ | LRRK2_bnPCR_ID2 | CCCTGGTGTGCCC | 6/6 symmetric internal loop at −22_position (GCCAUC- |
| ID | 9209_count871_- | TCTGATGTTTTTC | CAAAAA); |
| NO: | 22_26_gID_01244 | GAAGAATTCTAC | 1/1 U/G wobble base pair at −7 position; |
| 20 | | AGTAGGACTGAG | 1/1 G/G mismatch at −6 position; |
| | | CACTGCCGAGCT | 0/1 asymmetric bulge at −4 position (-C); |
| | | GGGCAATCTTTGC | 1/0 asymmetric bulge at −2 position (A-); |
| | | AATCAAAAAAGC | 1/1 A/C mismatch at 0 position; |
| | | ATTGGGATACAG | 1/1 U/C mismatch at +4 position; |
| | | TGTGAAGAGCAG | 1/1 A/G mismatch at +13 position; |
| | | CA | 1/1 G/U wobble at +16 position; |
| | | | 6/6 symmetric internal loop at +26 position (GGGGAU- |
| | | | CGAAGA) |
| SEQ | LRRK2_bnPCR ID2 | CCCTGGTGTGCCC | 6/6 symmetric internal loop at −20 position (CAUCAU- |
| ID | 9209_count871_- | TCTGATGTTTTTT | UACUAC); |
| NO: | 20_26_gID_07422 | AGGGGATTCTAC | 1/1 U/G wobble base pair at −7 position; |
| 21 | | AGTAGGACTGAG | 1/1 G/G mismatch at −6 position; |
| | | CACTGCCGAGCT | 0/1 asymmetric bulge at −4 position (-C); |
| | | GGGCAATCTTTGC | 1/0 asymmetric bulge at −2 position (A-); |
| | | ATACTACGCAGC | 1/1 A/C mismatch at 0 position; |
| | | ATTGGGATACAG | 1/1 U/C mismatch at +4 position; |
| | | TGTGAAGAGCAG | 1/1 A/G mismatch at +13 position; |
| | | CA | 1/1 G/U wobble base pair at +16 position; |
| | | | 6/6 symmetric internal loop at +26 position (GGGGAU- |
| | | | UAGGGG) |
| SEQ | LRRK2_bnPCR_ID2 | CCCTGGTGTGCCC | 6/6 symmetric internal loop at −16 position (AUUGCA- |
| ID | 9209_count871_- | TCTGATGTTTTTC | CAUUUG); |
| NO: | 16_26_gID_01312 | TACAGATTCTACA | 1/1 U/G wobble base pair at −7 position; |
| 22 | | GTAGGACTGAGC | 1/1 G/G mismatch at −6 position; |
| | | ACTGCCGAGCTG | 0/1 aysmmetric bulge at −4 position (-C); |
| | | GGCAATCTTCATT | 1/0 asymmetric bulge at −2 position (A-); |
| | | TGGATGGCAGCA | 1/1 A/C mismatch at 0 position; |
| | | TTGGGATACAGT | 1/1 U/C mismatch at +4 position; |
| | | GTGAAGAGCAGC | 1/1 A/G mismatch at +13 position; 1/1 G/U wobble base |
| | | A | pair at +16 position; |
| | | | 2/2 symmetric bulge at +26 position (GG-AG); |
| | | | 3/3 symmetric bulge at +29 position (GAU-CUA) |
| SEQ | LRRK2_bnPCR_ID2 | CCCTGGTGTGCCC | 6/6 symmetric internal loop at −16 position (AUUGCA- |
| ID | 9209_count871_- | TCTGATGTTTTTA | GAGUCG); |
| NO: | 16_24_gID_00727 | TGGGGTGTCTAC | 1/1 U/G wobble base pair at −7 position; |
| 23 | | AGTAGGACTGAG | 1/1 G/G mismatch at −6 position; |
| | | CACTGCCGAGCT | 0/1 asymmetric bulge at −4 position (-C); |
| | | GGGCAATCTTGA | 1/0 asymmetric bulge at −2 position (A-); |
| | | GTCGGATGGCAG | 1/1 A/C mismatch at 0 position; |
| | | CATTGGGATACA | 1/1 U/C mismatch at +4 position; |
| | | GTGTGAAGAGCA | 1/1 A/G mismatch at +13 position; |
| | | GCA | 1/1 G/U wobble at +16 position; 6/6 symmetric internal |
| | | | loop at +24 position (AUGGGG-GGGGUG) |
| SEQ | LRRK2_bnPCR_ID2 | CCCTGGTGTGCCC | 6/6 symmetric internal loop at −16 position (AUUGCA- |
| ID | 9209_count871_- | TCTGATGTTTTTA | CUCCCA); |
| NO: | 16_22_gID_11427 | TCCGAAACATAC | 1/1 U/G wobble at −7 position; |
| 24 | | AGTAGGACTGAG | 1/1 G/G mismatch at −6 positon; |
| | | CACTGCCGAGCT | 0/1 asymmetric bulge at −4 position (-C); |
| | | GGGCAATCTTCTC | 1/0 asymmetric bulge at −2 position (A-); |
| | | CCAGATGGCAGC | 1/1 A/C mismatch at 0 position; |
| | | ATTGGGATACAG | 1/1 U/C mismatch at +4 position; |
| | | TGTGAAGAGCAG | 1/1 A/G mismatch at +13 position; |
| | | CA | 1/1 G/U wobble base pair at +16 position; |
| | | | 6/6 symmetric internal loop at +22 position (GAAUGG- |
| | | | GAAACA) |
| SEQ | LRRK2_bnPCR_ID2 | CCCTGGTGTGCCC | 6/6 symmetric internal loop at −10 position (AAGAUU- |
| ID | 9209_count871_- | TCTGATGTTTTTC | CUCAGG); |
| NO: | 10_26_gID_00091 | ATGAGATTCTAC | 1/1 U/G wobble base pair at −7 position; |
| 25 | | AGTAGGACTGAG | 1/1 G/G mismatch at −6 position; |
| | | CACTGCCGAGCT | 0/1 asymmetric bulge at −4 position (-C); |
| | | GGGCCTCAGGTG | 1/0 asymmetric bulge at −2 position (A-); |
| | | CAATGATGGCAG | 1/1 A/C mismatch at 0 position; |
| | | CATTGGGATACA | 1/1 U/C mismatch at +4 position; |
| | | GTGTGAAGAGCA | 1/1 A/G mismatch at +13 position; |
| | | GCA | 1/1 G/U wobble base pair at +16 position; |
| | | | 6/6 symmetric internal loop at +26 position (GGGGAU- |
| | | | CAUGAG) |
| SEQ | LRRK2_bnPCR_ID2 | CCCTGGTGTGCCC | 4/4 symmetric bulge at −9 position (AUUG-GUUC); |
| ID | 9209_count871_- | TCTGATGTTTTTT | 1/1 U/G wobble base pair at −7 position; |
| NO: | 8_26_gID_11298 | ACAGAATTCTAC | 1/1 G/G mismatch at −6 position; |
| 26 | | AGTAGGACTGAG | 0/1 asymmetric bulge at −4 position (-C); |
| | | CACTGCCGAGCT | 1/0 asymmetric bulge at −2 position (A-); |
| | | GGGGTTCCTTTGC | 1/1 A/C mismatch at 0 position; |
| | | AATGATGGCAGC | 1/1 U/C mismatch at +4 position; |
| | | ATTGGGATACAG | 1/1 A/G mismatch at +13 position; |
| | | TGTGAAGAGCAG | 1/1 G/U wobble base pair at +16 position; |
| | | CA | 6/6 symmetric internal loop at +26 position (GGGGAU- |
| | | | UACAGA) |
| SEQ | LRRK2_bnPCR_ID2 | CCCTGGTGTGCCC | 6/6 symmetric internal loop at −20 position (CAUCAU- |
| ID | 8134_count1700_- | TCTGATGTTTTTT | UACUAC); 1/1 A/C mismatch at 0 position; |
| NO: | 20_26_gID_06283 | AGGGGATTCTAC | 1/1 C/C mismatch at +2 position; |
| 27 | | CGCAGTACTACC | 1/1 U/G wobble base pair at +5 position; |
| | | CGATCCCGTAGTC | 3/3 symmetric bulge at +7 position (CUC-ACC); |
| | | AGCAATCTTTGCA | 1/1 U/C mismatch at +18 position; |
| | | TACTACGCAGCA | 6/6 symmetric internal loop at +26 position (GGGGAU- |
| | | TTGGGATACAGT | UAGGGG) |
| | | GTGAAGAGCAGC | |
| | | A | |
| SEQ | LRRK2_bnPCR_ID2 | CCCTGGTGTGCCC | 1/1 U/G wobble base pair at −7 position; |
| ID | 4310_count1321_ | TCTGATGTTTTTA | 1/1 G/G mismatch at −6 position; |
| NO: | NoLoops_gID_110 | TCCCCATTCTAGG | 1/1 U/G wobble base pair at −3 position; |
| 28 | 60 | GCAGTAGGGTGC | 1/1 A/C mismatch at 0 position; |
| | | ACTGCCGTGGTG | 1/1 U/C mismatch at +4 position; |
| | | GGCAATCTTTGCA | 1/1 U/U mismatch at +8 position; |
| | | ATGATGGCAGCA | 2/2 symmetric bulge at +10 position (AG-GG); |
| | | TTGGGATACAGT | 1/1 U/G wobble base pair at +18 position; |
| | | GTGAAGAGCAGC | 1/1 G/G mismatch at +19 position |
| | | A | |
| SEQ | LRRK2_bnPCR_ID2 | CCCTGGTGTGCCC | 6/6 symmetric internal loop at −14 position (UGCAAA- |
| ID | 4310_count1321_- | TCTGATGTTTTTT | AAACGU); |
| NO: | 14_26_gID_02264 | AGGGGATTCTAG | 1/1 U/G wobble base pair at −7 position; |
| 29 | | GGCAGTAGGGTG | 1/1 G/G mismatch at −6 position; |
| | | CACTGCCGTGGT | 1/1 U/G wobble base pair at −3 position; |
| | | GGGCAATCAAAC | 1/1 A/C mismatch at 0 position; |
| | | GTATGATGGCAG | 1/1 U/C mismatch at +4 position; |
| | | CATTGGGATACA | 1/1 U/U mismatch at +8 position; |
| | | GTGTGAAGAGCA | 2/2 symmetric bulge at +10 position (AG-GG); |
| | | GCA | 1/1 U/G wobble at +18 position; |
| | | | 1/1 G/G mismatch at +19 position; |
| | | | 6/6 symmetric internal loop at +26 position (GGGGAU- |
| | | | UAGGGG) |
| SEQ | LRRK2_bnPCR_ID2 | CCCTGGTGTGCCC | 1/1 U/G wobble base pair at −3 position; |
| ID | 3393_count2397_ | TCTGATGTTTTTA | 1/1 A/C mismatch at 0 position; |
| NO: | NoLoops_gID_096 | TCCCCATTCTACC | 1/1 C/C mismatch at +2 position; |
| 30 | 25 | GCTGTGCTGGGC | 1/1 U/G wobble base pair at +8 position; |
| | | AATCCCGTGGTC | 1/1 U/G wobble base pair at +12 position; |
| | | AGCAATCTTTGCA | 1/1 U/U mismatch at +15 position; |
| | | ATGATGGCAGCA | 1/1 U/C mismatch at +18 position |
| | | TTGGGATACAGT | |
| | | GTGAAGAGCAGC | |
| | | A | |
| SEQ | LRRK2_bnPCR_ID2 | CCCTGGTGTGCCC | 6/6 symmetric internal loop at −20 position (CAUCAU- |
| ID | 3393_count2397_- | TCTGATGTTTTTT | UACUAC); |
| NO: | 20_26_gID_06774 | AGGGGATTCTAC | 1/1 U/G wobble base pair at −3 position; |
| 31 | | CGCTGTGCTGGG | 1/1 A/C mismatch at 0 position; |
| | | CAATCCCGTGGTC | 1/1 C/C mismatch at +2 position; |
| | | AGCAATCTTTGCA | 1/1 U/G wobble base pair at +8 position; |
| | | TACTACGCAGCA | 1/1 U/G wobble base pair at +12 position; |
| | | TTGGGATACAGT | 1/1 U/U mismatch at +15 position; |
| | | GTGAAGAGCAGC | 1/1 U/C mismatch at +18 position; |
| | | A | 6/6 symmetric internal loop at +26 position (GGGGAU- |
| | | | UAGGGG) |
| SEQ | LRRK2_bnPCR ID2 | CCCTGGTGTGCCC | 6/6 symmetric internal loop at −14 position (UGCAAA- |
| ID | 3393_count2397_- | TCTGATGTTTTTT | AAACGU); |
| NO: | 14_26_gID_06456 | AGGGGATTCTAC | 1/1 U/G wobble base pair at −3 position; |
| 32 | | CGCTGTGCTGGG | 1/1 A/C mismatch at 0 position; |
| | | CAATCCCGTGGTC | 1/1 C/C mismatch at +2 position; |
| | | AGCAATCAAACG | 1/1 U/G wobble base pair at +8 position; |
| | | TATGATGGCAGC | 1/1 U/G wobble base pair at +12 position; |
| | | ATTGGGATACAG | 1/1 U/G mismatch at +15 position; |
| | | TGTGAAGAGCAG | 1/1 U/C mismatch at +18 position; |
| | | CA | 6/6 symmetric internal loop at +26 position (GGGGAU- |
| | | | UAGGGG) |
| SEQ | LRRK2_bnPCR_ID2 | CCCTGGTGTGCCC | 1/1 U/G wobble at −3 position; |
| ID | 2759_count1590_- | TCTGATGTTTTTA | 1/1 A/C mismatch at 0 position; |
| NO: | NoLoops_gID_06335 | TCCCCATTCTACA | 1/1 U/G wobble base pair at +4 position; |
| 33 | | ACAGTACGGTGA | 5/5 symmetric internal loop at +6 position (GCUCA- |
| | | AGTGCCGTGGTC | GGUGA); |
| | | AGCAATCTTTGCA | 1/1 C/A mismatch at +17 position |
| | | ATGATGGCAGCA | |
| | | TTGGGATACAGT | |
| | | GTGAAGAGCAGC | |
| | | A | |
| SEQ | LRRK2_bnPCR_ID2 | CCCTGGTGTGCCC | 6/6 symmetric internal loop at −14 position (UGCAAA- |
| ID | 2759_count1590_- | TCTGATGTTTTTT | AAACGU); |
| NO: | 14_26_gID_08476 | AGGGGATTCTAC | 1/1 U/G wobble base pair at −3 position; |
| 34 | | AACAGTACGGTG | 1/1 A/C mismatch at 0 position; |
| | | AAGTGCCGTGGT | 1/1 U/G wobble base pair at +4 position; |
| | | CAGCAATCAAAC | 5/5 symmetric internal loop at +6 position (GCUCA- |
| | | GTATGATGGCAG | GGUGA); |
| | | CATTGGGATACA | 1/1 C/A mismatch at +17 position; |
| | | GTGTGAAGAGCA | 6/6 symmetric internal loop at +26 position (GGGGAU- |
| | | GCA | UAGGGG) |
| SEQ | LRRK2_bnPCR ID2 | CCCTGGTGTGCCC | 9/9 symmetric internal loop at −19 position |
| ID | 2357_count844_- | TCTGATGTTTTTC | (GCCAUCAUU-CAUUAUUUG); |
| NO: | 22_26_gID_02673 | TAGATATTCTACG | 1/1 C/A mismatch at −8 position; |
| 35 | | GCAGTTCATAGC | 1/1 A/C mismatch at 0 position; |
| | | AATCCCGTAGTC | 1/1 C/C mismatch at +2 position; |
| | | AACAATCTTTGCC | 2/2 symmetric bulge at +9 position (CA-AU); |
| | | ATTATTTGAGCAT | 1/1 U/G mismatch at +12 position; |
| | | TGGGATACAGTG | 1/1 U/G wobble base pair at +18 position; |
| | | TGAAGAGCAGCA | 1/1 G/U wobble base pair at +26 position; |
| | | | 5/5 symmetric internal loop at +27 position (GGGAU- |
| | | | CUAGA) |
| SEQ | LRRK2_bnPCR_ID2 | CCCTGGTGTGCCC | 8/8 symmetric internal loop at −12 position |
| ID | 2357_count844_- | TCTGATGTTTTTC | (UGCAAAGA-GGGUCCCC); |
| NO: | 12_26_gID_07465 | GGAGGATTCTAC | 1/1 C/A mismatch at −8 position; |
| 36 | | GGCAGTTCATAG | 1/1 A/C mismatch at 0 position; |
| | | CAATCCCGTAGTC | 1/1 C/C mismatch at +2 position; |
| | | AACAAGGGTCCC | 2/2 symmetric bulge at +9 position (CA-AU); |
| | | CATGATGGCAGC | 1/1 U/U mismatch at +12 position; |
| | | ATTGGGATACAG | 1/1 U/G wobble base pair at +18 position; |
| | | TGTGAAGAGCAG | 6/6 symmetric internal loop at +26 position (GGGGAU- |
| | | CA | CGGAGG) |
| SEQ | LRRK2_bnPCR_ID2 | CCCTGGTGTGCCC | 8/8 symmetric internal loop at −12 position |
| ID | 2357_count844_- | TCTGATGTTTTTA | (UGCAAAGA-CAAUAUCC); |
| NO: | 12_18_gID_11904 | TCCCCATCACCCT | 1/1 C/A mismatch at −8 position; |
| 37 | | GCAGTTCATAGC | 1/1 A/C mismatch at 0 position; |
| | | AATCCCGTAGTC | 1/1 C/C mismatch at +2 position; |
| | | AACAACAATATC | 2/2 symmetric bulge at +9 position (CA-AU); |
| | | CATGATGGCAGC | 1/1 U/U mismatch at +12 position; |
| | | ATTGGGATACAG | 6/6 symmetric internal loop at +18 position (UGUAGA- |
| | | TGTGAAGAGCAG | CACCCU) |
| | | CA | |
| SEQ | LRRK2_bnPCR ID1 | CCCTGGTGTGCCC | 6/6 symmetric internal loop at −22 position (GCCAUC- |
| ID | 6690_count1976_- | TCTGATGTTTTTC | AAGCGA); |
| NO: | 22_26_gID_01893 | TGGTGATTCTACA | 1/1 C/A mismatch at −8 position; |
| 38 | | ACAGTACTGAGC | 10/10 symmetric internal loop at −4 −> 5 position |
| | | TATCCCGAATTCA | (CUACAGCAUU-UAUCCCGAAU); |
| | | ACAATCTTTGCAA | |
| | | TAAGCGAAGCAT | |
| | | TGGGATACAGTG | |
| | | TGAAGAGCAGCA | 1/1 C/A mismatch at +17 position; |
| | | | 1/1 G/G mismatch at +26 position; |
| | | | 1/1 G/U wobble base pair at +27 position; |
| | | | 4/4 symmetric bulge at +28 position (GGAU-CUGG) |
| SEQ | LRRK2_bnPCR_ID1 | CCCTGGTGTGCCC | 6/6 symmetric internal loop at −22 position (GCCAUC- |
| ID | 6690_count1976_- | TCTGATGTTTTTA | CAAUCA); |
| NO: | 22_16_gID_06801 | TCCCCATTCGAAA | 1/1 C/A mismatch at −8 position; |
| 39 | | AAAGTACTGAGC | 10/10 symmetrica internal loop at −4 −> 5 position |
| | | TATCCCGAATTCA | (CUACAGCAUU-UAUCCCGAAU); |
| | | ACAATCTTTGCAA | 6/6 symmetric internal loop at +16 position (GCUGUA- |
| | | TCAATCAAGCATT | GAAAAA) |
| | | GGGATACAGTGT | |
| | | GAAGAGCAGCA | |
| SEQ | LRRK2_bnPCR ID1 | CCCTGGTGTGCCC | 6/6 symmetric internal loop at −16 position (AUUGCA- |
| ID | 6690_count1976_- | TCTGATGTTTTTA | CUAUUA); |
| NO: | 16_16_gID_10719 | TCCCCATTCGAAT | 1/1 C/A mismatch at −8 position; |
| 40 | | AAAGTACTGAGC | 10/10 symmetric internal loop at −4 −> 5 position |
| | | TATCCCGAATTCA | (CUACAGCAUU-UAUCCCGAAU); |
| | | ACAATCTTCTATT | 6/6 symmetric internal loop at +16 position (GCUGUA- |
| | | AGATGGCAGCAT | GAAUAA) |
| | | TGGGATACAGTG | |
| | | TGAAGAGCAGCA | |
| SEQ | LRRK2_bnPCR_ID1 | CCCTGGTGTGCCC | 1/1 C/U mismatch at −8 position; |
| ID | 4524_count2063_ | TCTGATGTTTTTA | 1/1 U/G wobble base pair at −3 position; |
| NO: | NoLoops_gID_011 | TCCCCATTCTACA | 1/1 A/C mismatch at 0 position; |
| 41 | 59 | GCAGTAGTGTGC | 1/1 U/g wobble base pair at +4 position; |
| | | AGTGCCGTGGTC | 1/1 U/U mismatch at +8 position; |
| | | ATCAATCTTTGCA | 1/1 G/G mismatch at +11 position |
| | | ATGATGGCAGCA | |
| | | TTGGGATACAGT | |
| | | GTGAAGAGCAGC | |
| | | A | |
| SEQ | LRRK2_bnPCR_ID1 | CCCTGGTGTGCCC | 6/6 symmetric internal loop at −14 position (UGCAAA- |
| ID | 4524_count2063_- | TCTGATGTTTTTT | AAACGU); |
| NO: | 14_26_gID_05349 | AGGGGATTCTAC | 1/1 C/U mismatch at −8 position; |
| 42 | | AGCAGTAGTGTG | 1/1 U/G wobble base pair at −3 position; |
| | | CAGTGCCGTGGT | 1/1 A/C mismatch at 0 position; |
| | | CATCAATCAAAC | 1/1 U/G wobble base pair at +4 position; |
| | | GTATGATGGCAG | 1/1 U/U mismatch at +8 position; |
| | | CATTGGGATACA | 1/1 G/G mismatch at +11 position; |
| | | GTGTGAAGAGCA | 6/6 symmetric internal loop at +26 position (GGGGAU- |
| | | GCA | UAGGGG) |
| SEQ | LRRK2_bnPCR_ID8 | CCCTGGTGTGCCC | 6/6 symmetric internal loop at −22 position (GCCAUC- |
| ID | 030_count919_- | TCTGATGTTTTTC | AAGAGA); |
| NO: | 22_26_gID_12156 | AGTAGCTTCTACA | 1/1 U/G wobble base pair at −3 position; |
| 43 | | GCAGTTCGGAGG | 1/1 A/A mismatch at −2 position; |
| | | AATCCCGAGGTC | 1/1 A/C mismatch at 0 position; |
| | | AGCAATCTTTGCA | 1/1 C/C mismatch at +2 position; |
| | | ATAAGAGAAGCA | 1/1 G/G mismatch at +6 position; |
| | | TTGGGATACAGT | 3/3 symmetric bulge at +10 position (AGU-UCG); |
| | | GTGAAGAGCAGC | 7/7 symmetric internal loop at +25 position |
| | | A | (UGGGGAU-CAGUAGC) |
| SEQ | LRRK2_bnPCR_ID8 | CCCTGGTGTGCCC | 6/6 symmetric internal loop at −20 position (CAUCAU- |
| ID | 030_count919_- | TCTGATGTTTTTT | UACUAC); |
| NO: | 20_26_gID_00460 | AGGGGCTTCTAC | 1/1 U/G wobble base pair at −3 position; |
| 44 | | AGCAGTTCGGAG | 1/1 A/A mismatch at −2 position; |
| | | GAATCCCGAGGT | 1/1 A/C mismatch at 0 position; |
| | | CAGCAATCTTTGC | 1/1 C/C mismatch at +2 position; 1/1 G/G mismatch at +6 |
| | | ATACTACGCAGC | position; |
| | | ATTGGGATACAG | 3/3 symmetric bulge at +10 position (AGU-UCG); |
| | | TGTGAAGAGCAG | 7/7 symmetric internal loop at +25 position |
| | | CA | (UGGGGAU-UAGGGGC) |
| SEQ | LRRK2_bnPCR_ID8 | CCCTGGTGTGCCC | 6/6 symmetric internal loop at −18 position (UCAUUG- |
| ID | 030_count919_- | TCTGATGTTTTTA | GUAGCU); |
| NO: | 18_24_gID_04981 | TGGAACGTCTAC | 1/1 U/G wobble base pair at −3 position; |
| 45 | | AGCAGTTCGGAG | 1/1 A/A mismatch at −2 position; |
| | | GAATCCCGAGGT | 1/1 A/C mismatch at 0 position; |
| | | CAGCAATCTTTGG | 1/1 C/C mismatch at +2 position; |
| | | TAGCTTGGCAGC | 1/1 G/G mismatch at +6 position; |
| | | ATTGGGATACAG | 3/3 symmetric bulge at +10 position (AGU-UCG); |
| | | TGTGAAGAGCAG | 6/6 symmetric internal loop at +24 position (AUGGGG- |
| | | CA | GGAACG) |
| SEQ | LRRK2_bnPCR_ID8 | CCCTGGTGTGCCC | 6/6 symmetric internal loop at −18 position (UCAUUG- |
| ID | 030_count919_- | TCTGATGTTTTTA | GUCUUC); |
| NO: | 18_22_gID_06761 | TCCAGCGGGTAC | 1/1 U/G wobble base pair at −3 position; |
| 46 | | AGCAGTTCGGAG | 1/1 A/A mismatch at −2 position; |
| | | GAATCCCGAGGT | 1/1 A/C mismatch at 0 position; |
| | | CAGCAATCTTTGG | 1/1 C/C mismatch at +2 position; |
| | | TCTTCTGGCAGCA | 1/1 G/G mismatch at +6 position; |
| | | TTGGGATACAGT | 3/3 symmetric bulge at +10 position (AGU-UCG); |
| | | GTGAAGAGCAGC | 6/6 symmetric internal loop at +22 position (GAAUGG- |
| | | A | AGCGGG) |
| SEQ | LRRK2_bnPCR_ID8 | CCCTGGTGTGCCC | 6/6 symmetric internal loop at −16 position (AUUGCA- |
| ID | 030_count919_- | TCTGATGTTTCAA | ACCCUG); |
| NO: | 16_28_gID_07038 | CGGCCCTTCTACA | 1/1 U/G wobble base pair at −3 position; |
| 47 | | GCAGTTCGGAGG | 1/1 A/A mismatch at −2 position; |
| | | AATCCCGAGGTC | 1/1 A/C mismatch at 0 position; |
| | | AGCAATCTTACCC | 1/1 C/C mismatch at +2 position; |
| | | TGGATGGCAGCA | 1/1 G/G mismatch at +6 position; |
| | | TTGGGATACAGT | 3/3 symmetric bulge at +10 position (AGU-UCG); |
| | | GTGAAGAGCAGC | 1/0 asymmetric bulge at +25 position (U-); |
| | | A | 5/6 asymmetric internal loop at +29 position (GAUAA- |
| | | | CAACGG) |
| SEQ | LRRK2_bnPCR_ID8 | CCCTGGTGTGCCC | 6/6 symmetric internal loop at −16 position (AUUGCA- |
| ID | 030_count919_- | TCTGATGTTTTTA | AAAUUA); |
| NO: | 16_24_gID_09086 | TTGAGCCTCTACA | 1/1 U/G wobble base pair at −3 position; |
| 48 | | GCAGTTCGGAGG | 1/1 A/A mismatch at −2 position; |
| | | AATCCCGAGGTC | 1/1 A/C mismatch at 0 position; |
| | | AGCAATCTTAAA | 1/1 C/C mismatch at +2 position; |
| | | TTAGATGGCAGC | 1/1 G/G mismatch at +6 position; |
| | | ATTGGGATACAG | 3/3 symmetric bulge at +10 position (AGU-UCG); |
| | | TGTGAAGAGCAG | 5/5 symmetric internal loop at +24 position (AUGGG- |
| | | CA | GAGCC); |
| | | | 1/1 G/U wobble base pair at +29 position |
| SEQ | LRRK2_bnPCR_ID8 | CCCTGGTGTGCCC | 6/6 symmetric internal loop at −14 position (UGCAAA- |
| ID | 030_count919_- | TCTGATGTTTTTT | AAACGU); |
| NO: | 14_26_gID_07899 | AGGGGCTTCTAC | 1/1 U/G wobble base pair at −3 position; |
| 49 | | AGCAGTTCGGAG | 1/1 A/A mismatch at −2 position; |
| | | GAATCCCGAGGT | 1/1 A/C mismatch at 0 position; |
| | | CAGCAATCAAAC | 1/1 C/C mismatch at +2 position; |
| | | GTATGATGGCAG | 1/1 G/G mismatch at +6 position; |
| | | CATTGGGATACA | 3/3 symmetric bulge at +10 position (AGU-UCG); |
| | | GTGTGAAGAGCA | 7/7 symmetric internal loop at +25 position |
| | | GCA | (UGGGGAU-UAGGGGC) |
| SEQ | LRRK2_bnPCR_ID8 | CCCTGGTGTGCCC | 6/6 symmetric internal loop at −12 position (CAAAGA- |
| ID | 030_count919_- | TCTGATGTTTTTA | GACUAA); |
| NO: | 12_24_gID_03363 | TGAGGCGTCTAC | 1/1 U/G wobble base pair at −3 position; |
| 50 | | AGCAGTTCGGAG | 1/1 A/A mismatch at −2 position; |
| | | GAATCCCGAGGT | 1/1 A/C mismatch at 0 position; |
| | | CAGCAAGACTAA | 1/1 C/C mismatch at +2 position; |
| | | CAATGATGGCAG | 1/1 G/G mismatch at +6 position; |
| | | CATTGGGATACA | 3/3 symmetric bulge at +10 position (AGU-UCG); |
| | | GTGTGAAGAGCA | 6/6 symmetric internal loop at +24 position (AUGGGG- |
| | | GCA | GAGGCG) |
| SEQ | LRRK2_bnPCR_ID8 | CCCTGGTGTGCCC | 6/6 symmetric internal loop at −10 position (AAGAUU- |
| ID | 030_count919_- | TCTGATGTTTTTA | UUAGCC); |
| NO: | 10_24_gID_09672 | TTGAGCCTCTACA | 1/1 U/G wobble base pair at −3 position; |
| 51 | | GCAGTTCGGAGG | 1/1 A/A mismatch at −2 position; |
| | | AATCCCGAGGTC | 1/1 A/C mismatch at 0 position; |
| | | AGCTTAGCCTGC | 1/1 C/C mismatch at +2 position; |
| | | AATGATGGCAGC | 1/1 G/G mismatch at +6 position; |
| | | ATTGGGATACAG | 3/3 symmetric bulge (AGU-UCG); |
| | | TGTGAAGAGCAG | 5/5 symmetric internal loop at +24 position (AUGGG- |
| | | CA | GAGCC); |
| | | | 1/1 G/U wobble base pair at +29 position |
| SEQ | ML_generative_0284 | ATTCTACGGCAGT | 2/2 symmetric bulge at position −8 (GC-UG); |
| ID | | ACTGGGACATCC | −1/1 U/G wobble base pair at −7 position; 1/1 U/G |
| NO: | | CGTGGTCGTGAA | wobble base pair at −3 position; |
| 52 | | TCTTTGCA | 5/4 asymmetric internal loop at 0 position spanning + 4 |
| | | | position (ACGAU-UCCC); |
| | | | 0/1 asymmetric bulge at position +6 (A); |
| | | | 8_1/1 U/G wobble base pair at +8 position; |
| | | | 1/1 U/G wobble base pair at +18 position |
| SEQ | ML_generative_0291 | ATTCTACAGCAG | 1/1 G/A mismatch at −9 position; |
| ID | | GACTGTTCAGTCC | 1/1 U/G wobble base pair at −7 position; |
| NO: | | CGTAGTCGGAAA | 1/1 A/C mismatch at 0 position; |
| 53 | | TCTTTGCA | 1/1 C/C mismatch at +2 position; |
| | | | 1/1 U/G wobble base at +4 position; |
| | | | 2/2 symmetric bulge at +7 position (CU-UU); |
| | | | 1/1 A/G mismatch at +13 position |
| SEQ | ML_generative_0157 | ATTCTACAGCAG | 1/1 C/C mismatch at −8 position; |
| ID | | GGCTCAGACATC | 1/1 U/G wobble base pair at −7 position; |
| NO | | CCGTGGTCGCCA | 1/1 U/G wobble base pair at −3 position; |
| 54 | | ATCTTTGCA | 5/4 asymmetric internal loop at 0 position spanning +4 |
| | | | position (AGCAU-UCCC); |
| | | | 0/1 asymmetric bulge at +6 position (A); |
| | | | 1/1 C/C mismatch at +9 position; |
| | | | 1/1 U/G wobble base pair at +12 position; |
| | | | 1/1 A/G mismatch at +13 position |
| SEQ | ML_generative_0224 | ATTCTACAGCAA | 1/1 U/U mismatch at −11 position; |
| ID | | GACTGGGACATC | 1/1 U/G wobble base pair at −7 position; |
| NO: | | CCGTGGTCGGCA | 1/1 U/G wobble base pair at −3 position; |
| 55 | | TTCTTTGCA | 5/4 asymmetric internal loop at 0 position spanning +4 |
| | | | position (AGCAU-UCCC); |
| | | | 0/1 asymmetric bulge at +6 position (A); |
| | | | 1/1 U/G wobble base pair at +8 position; |
| | | | 2/2 symmetric bulge at +13 position (AC-AG) |
| SEQ | ML_generative_0156 | ATTCTACAGCAGT | 2/2 symmetric bulge at −9 position (UG-AU); |
| ID | | ACTCAGGAAATC | 1/1 U/G wobble base pair at −7 position; |
| NO: | | CGTGGTCGGATA | 1/1 U/G wobble base pair at −3 position; |
| 56 | | TCTTTGCA | 1/1 A/C mismatch at 0 position; |
| | | | 2/2 symmetric bulge at +2 position (CA-AU); |
| | | | 1/1 G/G mismatch at +6 position; |
| | | | 1/1 C/C mismatch at + 9 position |
| SEQ | ML_generative_0469 | ATTCTACAGCAGT | 2/2 symmetric bulge at −8 position (GC-UA); |
| ID | | ACTGTTCAGTTCC | 1/1 U/G wobble base pair at −3 position; |
| NO: | | GAGGTCATAAAT | 1/1 A/A mismatch at −2 position; |
| 57 | | CTTTGCA | 1/1 A/C mismatch at 0 position; |
| | | | 1/1 C/U mismatch at +2 position; |
| | | | 1/1 U/G wobble base pair at +4 position; |
| | | | 2/2 symmetric bulge at +7 position (CU-UU) |
| SEQ | ML_generative_0579 | ATTCTACAGCAG | 1/1 A/G mismatch at −14 position; |
| ID | | GACTGGGCTCTCC | 1/1 C/U mismatch at −8 position; |
| NO: | | CGTGGTCATCAAT | 1/1 U/G wobble base pair at −3 position; |
| 58 | | CGTTGCA | 6/6 symmetric internal loop at 0 position spanning +5 |
| | | | position (AGCAUU-UCUCCC); |
| | | | 1/1 U/G wobble base pair at +8 position; |
| | | | 1/1 A/G mismatch at +13 position |
| SEQ | ML_generative_0278 | ATTCTACAGCGGT | 1/1 A/A mismatch at −12 position; |
| ID | | ACTGGGACATCC | 1/1 G/A mismatch at −9 position; |
| NO: | | CGTGGTCGGAAA | 1/1 U/G wobble base pair at −7 position; |
| 59 | | ACTTTGCA | 1/1 U/G wobble base pair at −3 position; |
| | | | 5/4 symmetric internal loop at 0 position spanning +4 |
| | | | position (AGCAU-UCCC); |
| | | | 0/1 asymmetric bulge at +6 position (A); |
| | | | 1/1 U/G wobble base pair at +8 position; |
| | | | 1/1 U/G wobble base pair at +15 position |
| SEQ | ML_generative_0464 | ATTCTATCGCAGT | 2/2 symmetric bulge at −7 position (CU-UU); |
| ID | | ATTGGGGAATCC | 1/1 U/G wobble base pair at −3 position; |
| NO: | | CGTGGTCTTCAAT | 1/1 A/C mismatch at 0 position; |
| 60 | | CTTTGCA | 1/1 C/C mismatch at +2 position; |
| | | | 1/1 G/G mismatch at +6 position; |
| | | | 1/1 U/G wobble base pair at +8 position; |
| | | | 1/1 G/U wobble base pair at +11 position; |
| | | | 1/1 U/C mismatch at +18 position; |
| | | | 1/1 G/U wobble base pair at +19 position |
| SEQ | ML_generative_0628 | ATTCTACAGCAGT | 1/2 asymmetric bulge at −8 position (C-AC); |
| ID | | ACTGGGCTTTCCC | 1/1 G/U wobble base pair at −6 position; |
| NO: | | GTGTTAACCAATC | 1/0 asymmetric bulge at −3 position (U); |
| 61 | | TTTGCA | 1/1 A/C mismatch at 0 position; |
| | | | 1/1 C/C mismatch at +2 position; |
| | | | 2/2 symmetric bulge at +4 position (UU-UU); |
| | | | 1/1 U/G wobble base pair at +8 position |
| SEQ | ML_generative_0427 | ATTCTACAGCAGT | 1/1 C/U mismatch at −8 position; |
| ID | | ACTGTGGAAATC | 1/1 U/G wobble base pair at −7 position; |
| NO: | | CGTGGTTGTCAAT | 1/1 G/U wobble base pair at −6 position; |
| 62 | | CTTTGCA | 1/1 U/G wobble base pair at −3 position; |
| | | | 1/1 A/C mismatch at 0 position; |
| | | | 2/2 symmetric bulge at +2 position (CA-AU); |
| | | | 1/1 G/G mismatch at +6 position; |
| | | | 1/1 U/U mismatch at +8 position |
| SEQ | ML_generative_0521 | ATTCTACAGCAG | 1/1 C/C mismatch at −8 position; |
| ID | | GACTCAGCACAG | 1/1 U/G wobble base pair at −7 position; |
| NO: | | CCGAAGTTGCCA | 1/1 G/U wobble base pair at −6 position; |
| 63 | | ATCTTTGCA | 1/1 A/A mismatch at −2 position; |
| | | | 1/1 A/C mismatch at 0 position; |
| | | | 2/2 symmetric bulge at +3 position (AU-CA); |
| | | | 1/1 C/C mismatch at +9 position; |
| | | | 1/1 A/G mismatch at +13 position |
| SEQ | ML_generative_0188 | GTTCTAGAGCAG | 1/1 U/G wobble base pair at −7 position; |
| ID | | GACTGTCCAGTCC | 1/1 A/C mismatch at 0 position; |
| NO: | | CGTAGTCGGCAA | 1/1 C/C mismatch at +2 position; |
| 64 | | TCTTTGCA | 1/1 U/G wobble base pair at +4 position; |
| | | | 2/2 symmetric bulge at +7 position (CU-UC); |
| | | | 1/1 A/G mismatch at +13 position; |
| | | | 1/1 G/G mismatch at +19 position; |
| | | | 1/1 U/G wobble base pair at +25 position |
| SEQ | ML_generative_0226 | ATTCTACGGCAGT | 1/1 U/G wobble base pair at −7 position; |
| ID | | ACTGGGGAAATC | 1/1 U/G wobble base pair at −3 position; |
| NO: | | CGTGGTCGGCAA | 1/1 A/C mismatch at 0 position; |
| 65 | | TCTTTGCA | 2/2 symmetric bulge at +2 position (CA-AU); |
| | | | 1/1 G/G mismatch at +6 position; |
| | | | 1/1 U/G wobble base pair at +8 position; |
| | | | 1/1 U/G wobble base pair at +18 position |
| SEQ | ML_generative_0008 | ATTCTACCGCAGT | 2/2 symmetric bulge at −7 position (CU-UU); |
| ID | | ACTGTGACATCCC | 1/1 U/G wobble base pair at −3 position; |
| NO: | | GTGGTCTTCAATC | 5/4 symmetric internal loop at 0 position spanning +4 |
| 66 | | TTTGCA | position (AGCAU-UCCC); |
| | | | 0/1 asymmetric bulge at +6 position (A); |
| | | | 1/1 U/U mismatch at +8 position; |
| | | | 1/1 U/C mismatch at +18 position |
| SEQ | ML_generative_0285 | ATTCTACAGCAG | 1/1 U/G wobble base pair at −7 position; |
| ID | | GAGTGTGCAGTC | 1/1 A/A mismatch at −5 position; |
| NO: | | CCGAAGACGGCA | 1/1 A/A mismatch at −2 position; |
| 67 | | ATCTTTGCA | 1/1 A/C mismatch at O position; |
| | | | 1/1 C/C mismatch at +2 position; |
| | | | 1/1 U/G wobble base pair at +4 position; |
| | | | 1/1 U/U mismatch at +8 position; |
| | | | 1/1 G/G mismatch at +11 position; |
| | | | 1/1 A/G mismatch at +13 position |
| SEQ | ML_generative_0058 | ATTCTACGGCAG | 1/1 U/C mismatch at −10 position; |
| ID | | GACTGGGGAAAT | 1/1 U/G wobble base pair at −7 position; |
| NO: | | CCGTGGTCGGCC | 1/1 U/G wobble base pair at −3 position; |
| 68 | | ATCTTTGCA | 1/1 A/C mismatch at 0 position; |
| | | | 2/2 symmetric bulge at +2 position (CA-AU); |
| | | | 1/1 G/G mismatch at +6 position; |
| | | | 1/1 U/G wobble base pair at +8 position; |
| | | | 1/1 A/G mismatch at +13 position; |
| | | | 1/1 U/G wobble base pair at +18 position |
| SEQ | ML_generative_0229 | ATTCTACAGCGGT | 1/1 C/U mismatch at −8 position; |
| ID | | ACTCACCAGTCCC | 1/1 A/A mismatch at −2 position; |
| NO: | | GAAGTCATCAAT | 1/1 A/C mismatch at 0 position; |
| 69 | | CTTTGCC | 1/1 C/C mismatch at +2 position; |
| | | | 1/1 U/G wobble base pair at +4 position; |
| | | | 3/3 symmetric bulge at +7 position (CUC-CAC); |
| | | | 1/1 U/G wobble base pair at +15 position |
| SEQ | ML_generative_0433 | ATTCTACCGCAGT | 1/1 U/G wobble base pair at −7 position; |
| ID | | ACTGCGAAATCC | 1/1 U/G wobble base pair at −3 position; |
| NO: | | CGAGGTCGGCAA | 1/1 A/A mismatch at −2 position; |
| 70 | | TCTTTGCA | 1/1 A/C mismatch at 0 position; |
| | | | 1/1 C/C mismatch at +2 position; |
| | | | 1/1 G/A mismatch at +6 position; |
| | | | 1/1 U/C mismatch at +8 position; |
| | | | 1/1 U/C mismatch at +18 position |
| SEQ | ML_generative_0430 | ATTCTACTGCAGG | 1/1 U/U mismatch at −11position; |
| ID | | ACTTAGGAGTCC | 1/1 U/G wobble base pair at −7 position; |
| NO: | | CGTGGTCGGCATT | 1/1 U/G wobble base pair at −3 position; |
| 71 | | CTTTGCA | 1/1 A/C mismatch at 0 position; |
| | | | 1/1 C/C mismatch at +2 position; |
| | | | 1/1 U/G wobble base pair at +4 position; |
| | | | 1/1 G/G mismatch at +6 position; |
| | | | 1/1 C/U mismatch at +9 position; |
| | | | 1/1 A/G mismatch at +13 position; |
| | | | 1/1 U/U mismatch at +18 position |
| SEQ | ML_generative_0183 | ATTCTAGGGCAG | 1/1 U/G wobble base pair at −7 position; |
| ID | | TACTGGGACATC | 1/1 U/G wobble base pair at −3 position; |
| NO: | | CCGTGGTCGGCA | 5/4 symmetric internal loop at 0 position spanning +4 |
| 72 | | ATCTTTGCA | position (AGCAU-UCCC); |
| | | | 0/1 asymmetric bulge at +6 position (A); |
| | | | 1/1 U/G wobble base pair at +8 positon; |
| | | | 1/1 U/G wobble base pair at +18 positon; |
| | | | 1/1 G/G mismatch at +19 position |
| SEQ | ML_generative_0099 | ATTCTACAGCAGT | 1/1 U/G wobble base pair at −7 position; |
| ID | | ACTCACCAGTCCC | 1/1 G/G mismatch at −6 position; |
| NO: | | GTGGTGGGCAAT | 1/1 U/G wobble base pair at −3 position; |
| 73 | | CTTTGCA | 1/1 A/C mismatch at 0 position; |
| | | | 1/1 C/C mismatch at +2 position; |
| | | | 1/1 U/G wobble base pair at +4 position; |
| | | | 3/3 symmetric bulge at +7 position (CUC-CAC) |
| SEQ | ML_generative_0073 | ATTCTACAGCAGT | 1/1 U/G wobble base pair at −7 position; |
| ID | | GCTCACCAGTCCC | 1/1 U/G wobble base pair at −3 position; |
| NO: | | GTGGTCGGCAAT | 1/1 A/C mismatch at 0 position; |
| 74 | | CTTTGCA | 1/1 C/C mismatch at +2 position; |
| | | | 1/1 U/G wobble base pair at +4 position; |
| | | | 3/3 symmetric bulge at +7 position (CUC-CAC); |
| | | | 1/1 U/G wobble base pair at +12 position |
| SEQ | ML_generative_0263 | ATTCTAGAGCAG | 1/1 C/U mismatch at −8 position; |
| ID | | TACTGGGGAGTT | 1/1 U/G wobble base pair at −7 position; |
| NO: | | CCGTGGTCGTCA | 1/1 U/G wobble base pair at −3 position; |
| 75 | | ATCTTTGCA | 1/1 A/C mismatch at 0 position; |
| | | | 1/1 C/U mismatch at +2 position; |
| | | | 1/1 U/G wobble base pair at +4 position; |
| | | | 1/1 G/G mismatch at +6 position; |
| | | | 1/1 U/G wobble base pair at +8 position; |
| | | | 1/1 G/G mismatch at +19 position |
| SEQ | ML_generative_0225 | ATTCTACAGCAG | 1/1 U/G wobble base pair at −7 position; |
| ID | | GAGTGGGCAGTC | 1/1 U/G wobble base pair at −3 position; |
| NO: | | CCGTGGTCGGCA | 1/1 A/C mismatch at 0 position; |
| 76 | | ATCTTTGCA | 1/1 C/C mismatch at +2 position; |
| | | | 1/1 U/G wobble base pair at +4 position; |
| | | | 1/1 U/G wobble base pair at +8 position; |
| | | | 1/1 G/G mismatch at +11 position; |
| | | | 1/1 A/G mismatch at +13 position |
| SEQ | ML_generative_0182 | ATTCTACGGCAG | 1/1 G/G mismatch at −9 position; |
| ID | | GACTGGGGAAAT | 1/1 U/G wobble base pair at −7 position; |
| NO: | | CCGTGGTCGGGA | 1/1 U/G wobble base pair at −3 position; |
| 77 | | ATCTTTGCA | 1/1 A/C mismatch at 0 position; |
| | | | 2/2 symmetric bulge at +2 position (CA-AU); |
| | | | 1/1 G/G mismatch at +6 position; |
| | | | 1/1 U/G wobble base pair at +8 position; |
| | | | 1/1 A/G mismatch at +13 position; |
| | | | 1/1 U/G wobble base pair at +18 position |
| SEQ | ML_generative_0060 | ATTCTACAGCAG | 1/1 U/G wobble base pair at −7 position; |
| ID | | GAGTGTGCAGTC | 1/1 U/G wobble base pair at −3 position; |
| NO: | | CCGTGGTCGGCA | 1/1 A/C mismatch at 0 position; |
| 78 | | ATCTTTGCA | 1/1 C/C mismatch at +2 position; |
| | | | 1/1 U/G wobble base pair at +4 position; |
| | | | 1/1 U/U mismatch at +8 position; |
| | | | 1/1 G/G mismatch at +11 position; |
| | | | 1/1 A/G mismatch at +13 position |
| SEQ | ML_generative_0532 | ATTCTACATCTGT | 1/1 U/G wobble base pair at −7 position; |
| ID | | ATTGGGACATCC | 1/1 U/G wobble base pair at −3 position; |
| NO: | | CGTGGTCGGCAA | 5/4 symmetric internal loop at 0 position spanning +4 |
| 79 | | TCTTTGCA | position (AGCAU-UCCC); |
| | | | 0/1 asymmetric bulge at +6 position (A); |
| | | | 1/1 U/G wobble base pair at +8 position; |
| | | | 1/1 G/U wobble base pair at +11 position; |
| | | | 1/1 U/U mismatch at +15 position; |
| | | | 1/1 C/U mismatch at +17 position |
| SEQ | ML_generative_0239 | ATTCTACAGCAA | 1/1 U/G wobble base pair at −7 position; |
| ID | | GACTGGGCAGTC | 1/1 A/A mismatch at −5 position; |
| NO: | | CCGTAGACGGCA | 1/1 A/C mismatch at 0 position; |
| 80 | | ATCTTTGCA | 1/1 C/C mismatch at +2 position; |
| | | | 1/1 U/G wobble base pair at +4 position; |
| | | | 1/1 U/G wobble base pair at +8 position; |
| | | | 2/2 symmetric bulge at +13 position (AC-AG) |
| SEQ | ML_generative_0125 | ATTCTAGAGCAG | 1/1 U/G wobble base pair at −7 position; |
| ID | | TACTGTGACATCC | 1/1 U/G wobble base pair at −3 position; |
| NO: | | CGTGGTCGGCAA | 5/4 symmetric internal loop at 0 position spanning +4 |
| 81 | | TCTTTGCA | position (AGCAU-UCCC); |
| | | | 0/1 asymmetric bulge at +6 position (A); |
| | | | 1/1 U/U mismatch at +8 position; |
| | | | 1/1 G/G mismatch at +19 position |
| SEQ | ML_generative_0343 | ATTCTATAGCAG | 1/1 C/A mismatch at −8 position; |
| ID | | GACTGAGCACTC | 1/1 U/G wobble base pair at −7 position; |
| NO: | | CCGTGGTCGACA | 1/1 U/G wobble base pair at −3 position; |
| 82 | | ATCTTTGCA | 1/1 A/C mismatch at 0 position; |
| | | | 3/3 symmetric bulge at +2 position (CAU-CUC); |
| | | | 1/1 A/G mismatch at +13 position; |
| | | | 1/1 G/U wobble base pair at +19 position |
| SEQ | ML_generative_0101 | ATTCTACCGCAGT | 1/1 U/G wobble base pair at −7 position; |
| ID | | ACTTATCAGTCCC | 1/1 U/G wobble base pair at −3 position; |
| NO: | | GTGGTCGGCAAT | 1/1 A/C mismatch at 0 position; |
| 83 | | CTTTGCA | 1/1 C/C mismatch at +2 position; |
| | | | 1/1 U/G wobble base pair at +4 position; |
| | | | 3/3 symmetric bulge at +7 position (CUC-UAU); |
| | | | 1/1 U/C mismatch at +18 position |
| SEQ | ML_generative_0439 | GTTCTACAGCAG | 1/1 U/G wobble base pair at −7 position; |
| ID | | GCTTGGGCAGTC | 1/1 U/G wobble base pair at −3 position; |
| NO: | | CCGTGGTCGGCA | 1/1 A/C mismatch at 0 position; |
| 84 | | ATCTTTGCA | 1/1 C/C mismatch at +2 position; |
| | | | 1/1 U/G wobble base pair at +4 position; |
| | | | 1/1 U/G wobble base pair at +8 positon; |
| | | | 1/1 G/U wobble base pair at +11 position; |
| | | | 2/2 symmetric bulge at +12 position (UA-GC); |
| | | | 1/1 U/G wobble base pair at +25 position |
| SEQ | ML_generative_0232 | ATTCTACAGCAGT | 1/1 C/A mismatch at −8 position; |
| ID | | ACTCAGCTTTCCC | 1/1 U/G wobble base pair at −7 position; |
| NO: | | GTGGTCGACAAT | 1/1 U/G wobble base pair at −3 position; |
| 85 | | CTTTGCA | 1/1 A/C mismatch at 0 position; |
| | | | 1/1 C/C mismatch at +2 position; |
| | | | 2/2 symmetric bulge at +4 position (UU-UU); |
| | | | 1/1 C/C mismatch at +9 position |
| SEQ | ML_generative_0088 | ATTCTACGGTAG | 1/1 U/G wobble base pair at −7 position; |
| ID | | GACGGAGCAGTC | 1/1 U/G wobble base pair at −3 position; |
| NO: | | CCGTGGTCGGCA | 1/1 A/C mismatch at 0 position; |
| 86 | | ATCTTTGCA | 1/1 C/C mismatch at +2 position; |
| | | | 1/1 U/G wobble base pair at +4 position; |
| | | | 1/1 A/G mismatch at +10 position; |
| | | | 1/1 A/G mismatch at +13 position; |
| | | | 1/1 G/U wobble base pair at +16 position; |
| | | | 1/1 U/G wobble base pair at +18 position |
| SEQ | ML_generative_0032 | ATTCTACAGCAGT | 1/1 U/G wobble base pair at −7 position; |
| ID | | ACTCACCAGTCCC | 1/1 U/G wobble base pair at −3 position; |
| NO: | | GTGGTCGGCAAT | 1/1 A/C mismatch at 0 position; |
| 87 | | CTTTGCA | 1/1 C/C mismatch at +2 position; |
| | | | 1/1 U/G wobble base pair at +4 position; |
| | | | 3/3 symmetric bulge at +7 position (CUC-CAC) |
| SEQ | ML_generative_0191 | ATTCTACAGCCGT | 1/1 U/G wobble base pair at −7 position; |
| ID | | ACAGAGCAGTCC | 1/1 A/A mismatch at −5 position; |
| NO: | | CGTGGACGGCAA | 1/1 U/G wobble base pair at −3 position; |
| 88 | | TCTTTGCA | 1/1 A/C mismatch at 0 position; |
| | | | 1/1 C/C mismatch at +2 position; |
| | | | 1/1 U/G wobble base pair at +4 position; |
| | | | 1/1 A/A mismatch at +10 position; |
| | | | 1/1 U/C mismatch at +15 position |
| SEQ | ML_generative_0057 | ATTCTACGGCAG | 1/1 U/G wobble base pair at −7 position; |
| ID | | GACTGGGCAGTC | 1/1 A/C mismatch at 0 position; |
| NO: | | CCGTAGTCGGCA | 1/1 C/C mismatch at +2 position; |
| 89 | | ATCTTTGCA | 1/1 U/G wobble base pair at +4 position; |
| | | | 1/1 U/G wobble base pair at +8 position; |
| | | | 1/1 A/G mismatch at +13 position; |
| | | | 1/1 U/G wobble base pair at +18 position |
| SEQ | ML_generative_0363 | ATTCTAGTGCAG | 1/1 U/G wobble base pair at −7 position; |
| ID | | GACTGGGACATC | 5/4 symmetric internal loop at 0 position spanning +4 |
| NO: | | CCGTAGTCGGCA | position (AGCAU-UCCC); |
| 90 | | ATCTTTGCA | 0/1 asymmetric bulge at +6 position (A); |
| | | | 1/1 U/G wobble base pair at +8 position; |
| | | | 1/1 A/G mismatch at +13 position; |
| | | | 2/2 symmetric bulge at +18 position (UG-GU) |
| SEQ | ML_generative_0048 | ATTCTACAGTGGT | 1/1 U/G wobble base pair at −7 position; |
| ID | | ACTCACCAGTCCC | 1/1 U/G wobble base pair at −3 position; |
| NO: | | GTGGTCGGCAAT | 1/1 A/C mismatch at 0 position; |
| 91 | | CTTTGCA | 1/1 C/C mismatch at +2 position; |
| | | | 1/1 U/G wobble base pair at +4 position; |
| | | | 3/3 symmetric bulge at +7 position (CUC-CAC); |
| | | | 1/1 U/G wobble base pair at +15 position; |
| | | | 1/1 G/U wobble base pair at +16 position |
| SEQ | ML_generative_0110 | ATTCTACAGCAGT | 1/1 C/C mismatch at −8 position; |
| ID | | ACTCAGAAGTTC | 1/1 U/G wobble base pair at −7 position; |
| NO: | | CGTGGTCGCCAA | 1/1 U/G wobble base pair at −3 position; |
| 92 | | TCTTTGCA | 1/1 A/C mismatch at 0 position; |
| | | | 1/1 C/U mismatch at +2 position; |
| | | | 1/1 U/G wobble base pair at +4 position; |
| | | | 1/1 G/A mismatch at +6 position; |
| | | | 1/1 C/C mismatch at +9 position |
| SEQ | ML_generative_0461 | ATTCTACAGCGGT | 1/1 A/G mismatch at −14 position; |
| ID | | ACTGTTTAATCCC | 1/1 U/G wobble base pair at −7 position; |
| NO: | | GTGGTCGGCAAT | 1/1 U/G wobble base pair at −3 position; |
| 93 | | CGTTGCA | 1/1 A/C mismatch at 0 position; |
| | | | 1/1 C/C mismatch at +2 position; |
| | | | 1/1 G/U wobble base pair at +6 position; |
| | | | 2/2 symmetric bulge at +7 position (CU-UU); |
| | | | 1/1 U/G wobble base pair at +15 position |
| SEQ | ML_generative_0098 | ATTCTATGGCAGT | 1/1 C/A mismatch at −8 position; |
| ID | | ACTGTGCAGTCCC | 1/1 U/G wobble base pair at −7 position; |
| NO: | | GTGGTCGACAAT | 1/1 U/G wobble base pair at −3 position; |
| 94 | | CTTTGCA | 1/1 A/C mismatch at 0 position; |
| | | | 1/1 C/C mismatch at +2 position; |
| | | | 1/1 U/G wobble base pair at +4 position; |
| | | | 1/1 U/U mismatch at +8 position; |
| | | | 1/1 U/G wobble base pair at +18 position; |
| | | | 1/1 G/U wobble base pair at +19 position |
| SEQ | ML_generative_0623 | ATTCTACAGGAG | 1/1 U/G wobble base pair at −7 position; |
| ID | | GACTGAGCAGTC | 1/2 asymmetric bulge at −6 position (G-UG); |
| NO: | | CCGAGTTGGGCA | 1/0 asymmetric bulge at −2 position (A); |
| 95 | | ATCTTTGCA | 1/1 A/C mismatch at 0 position; 1/1 C/C mismatch at +2 |
| | | | position; |
| | | | 1/1 U/G wobble base pair at +4 position; |
| | | | 1/1 A/G mismatch at +13 position; |
| | | | 1/1 G/G mismatch at +16 position |
| SEQ | ML_generative_0094 | ATTCTAGAGCAG | 1/1 U/G wobble base pair at −7 position; |
| ID | | TACTGCGCAAAT | 1/1 U/G wobble base pair at −3 position; |
| NO: | | CCGTGGTCGGCA | 1/1 A/C mismatch at 0 position; |
| 96 | | ATCTTTGCA | 2/2 symmetric bulge at +2 position (CA-AU); |
| | | | 1/1 U/C mismatch at +8 position; |
| | | | 1/1 G/G mismatch at +19 position |
| SEQ | ML_generative_0636 | ATTCTACAGCAG | 1/1 U/G wobble base pair at −7 position; |
| ID | | GACTGAGGAGTC | 1/1 A/C mismatch at −5 position; |
| NO: | | CCGAGGCCGGCA | 1/1 U/G wobble base pair at −3 position; |
| 97 | | ATCTTTGCA | 1/1 A/A mismatch at −2 position; |
| | | | 1/1 A/C mismatch at 0 position; |
| | | | 1/1 C/C mismatch at +2 position; |
| | | | 1/1 U/G wobble base pair at +4 position; |
| | | | 1/1 G/G mismatch at +6 position; |
| | | | 1/1 A/G mismatch at +13 position |
| SEQ | ML_generative_0324 | ATTCTACAGGAG | 1/1 G/G mismatch at −9 position; |
| ID | | TACTGATTAAATC | 1/1 U/G wobble base pair at −7 position; |
| NO: | | CGTGGTTGGGAA | 1/1 G/U wobble base pair at −6 position; |
| 98 | | TCTTTGCA | 1/1 U/G wobble base pair at −3 position; |
| | | | 1/1 A/C mismatch at 0 position; |
| | | | 2/2 symmetric bulge at +2 position (CA-AU); |
| | | | 1/1 G/U wobble base pair at +6 position; |
| | | | 1/1 C/U mismatch at +7 position; |
| | | | 1/1 G/G mismatch at +16 position |
| SEQ | ML_generative_0204 | ATTCTACAGGAG | 1/1 G/U wobble base pair at −9 position; |
| ID | | GACTGGGACATC | 1/1 C/C mismatch at −8 position; |
| NO: | | CCGTGGTCGCTA | 1/1 U/G wobble base pair at −7 position; |
| 99 | | ATCTTTGCA | 1/1 U/G wobble base pair at −3 position; |
| | | | 5/4 symmetric internal loop at 0 position spanning +4 |
| | | | position (AGCAU-UCCC); |
| | | | 0/1 asymmetric bulge at +6 position (A); |
| | | | 1/1 U/G wobble base pair at +8 position; |
| | | | 1/1 A/G mismatch at +13 position; |
| | | | 1/1 G/G mismatch at +16 position |
| SEQ | ML_generative_0140 | ATTCTACAGCCGT | 1/1 U/G wobble base pair at −7 position; |
| ID | | | 1/1 A/A mismatch at −5 position; |
| NO: | | ACAGGGCAGTCC | 1/1 U/G wobble base pair at −3 position; |
| 100 | | CGTGGACGGCAA | 1/1 A/C mismatch at 0 position; |
| | | TCTTTGCA | 1/1 C/C mismatch at +2 position; |
| | | | 1/1 U/G wobble base pair at +4 position; |
| | | | 1/1 U/G wobble base pair at +8 positon; |
| | | | 1/1 A/A mismatch at +10 position; |
| | | | 1/1 U/C mismatch at +15 position |
| SEQ | ML_generative_0222 | ATTCTACAGCAGT | 1/1 U/G wobble base pair at −7 position; |
| ID | | | 1/1 U/G wobble base pairt at −3 position; |
| NO: | | ACGGTCCAGTCC | 1/1 A/C mismatch at 0 position; |
| 101 | | CGTGGTCGGCAA | 1/1 C/C mismatch at +2 position; |
| | | TCTTTGCA | 1/1 U/G wobble base pair at +4 position; |
| | | | 2/2 symmetric bulge at +7 position (CU-UC); |
| | | | 1/1 A/G mismatch at +10 position |

Also disclosed herein are methods of treating a subject by administering a guide RNA targeting LRRK2 or a polynucleotide encoding the same. Administration of a guide RNA targeting LRRK2 as described herein can be used to treat a disease or condition associated with a mutation of LRRK2 as described herein (e.g. Parkinson's Disease or Crohn's Disease).

Pro-inflammatory signals upregulate LRRK2 expression in various immune cell types, suggesting that LRRK2 is a critical regulator in the immune response. Studies have found that both systemic and central nervous system (CNS) inflammation are involved in Parkinson's Disease's symptoms. Moreover, LRRK2 mutations associated with Parkinson's Disease modulate its expression levels in response to inflammatory stimuli. Many mutations in LRRK2 are associated with immune-related disorders such as inflammatory bowel disease such as Crohn's Disease. For example, both G2019S and N2081D increase LRRK2's kinase activity and are over-represented in Crohn's Disease patients in specific populations. Because of its critical role in these disorders, LRRK2 is an important therapeutic target for Parkinson's Disease and Crohn's Disease. In particular, many mutations, such as point mutations including G2019S, play roles in developing these diseases, making LRRK2 an attractive for therapeutic strategy such as RNA editing.

In some embodiments, the present disclosure provides compositions and methods of use thereof of guide RNAs that are capable of facilitating RNA editing of LRRK2. In some embodiments, a guide RNA of the present disclosure can target the following mutations in LRRK2: E10L, A30P, S52F, E46K, A53T, L119P, A211V, C228S, E334K, N363S, V366M, A419V, R506Q, N544E, N551K, A716V, M712V, I723V, P755L, R793M, I810V, K871E, Q923H, Q930R, R1067Q, S1096C, Q1111H, I1122V, A1151T, L1165P, I1192V, H1216R, S1228T, P1262A, R1325Q, I1371V, R1398H, T1410M, D1420N, R1441G, R1441H, A1442P, P1446L, V1450I, K1468E, R1483Q, R1514Q, P1542S, V1613A, R1628P, M1646T, S1647T, Y1699C, R1728H, R1728L, L1795F, M1869V, M1869T, L1870F, E1874X, R1941H, Y2006H, I2012T, G2019S, I2020T, T2031S, N2081D, T2141M, R2143H, Y2189C, T2356I, G2385R, V2390M, E2395K, M2397T, L2466H, or Q2490NfsX3. Said guide RNAs targeting a site in LRRK2 can be encoded by an engineered polynucleotide construct of the present disclosure.

Said guide RNAs targeting a site in LRRK2 can be encoded by an engineered polynucleotide construct of the present disclosure. An engineered guide RNA targeting LRRK2 can comprise a polynucleotide of any of the sequences recited in TABLE 1 provided in the present disclosure. An engineered guide RNA targeting LRRK2 can comprise a polynucleotide of any of the sequences recited in TABLE 2 provided in the present disclosure. An engineered guide RNA targeting LRRK2 can comprise a polynucleotide of any of the sequences recited in TABLE 3 provided in the present disclosure. An engineered guide RNA targeting LRRK2 can comprise a polynucleotide of any of the sequences recited in TABLE 4 provided in the present disclosure. An engineered guide RNA targeting LRRK2 can comprise a polynucleotide of any of the sequences recited in TABLE 5 provided in the present disclosure. An engineered guide RNA targeting LRRK2 can comprise a polynucleotide of any of the sequences recited in TABLE 6 provided in the present disclosure. An engineered guide RNA targeting LRRK2 can comprise a polynucleotide of any of the sequences recited in TABLE 7 provided in the present disclosure. An engineered guide RNA targeting LRRK2 can comprise a polynucleotide of any of the sequences recited in TABLE 8 provided in the present disclosure. An engineered guide RNA targeting LRRK2 can comprise a polynucleotide of any of the sequences recited in TABLE 9 provided in the present disclosure.

A guide RNA targeting LRRK2 can comprise any one of SEQ ID NO: 2-SEQ ID NO: 395 or SEQ ID NO: 398-SEQ ID NO: 472. In some examples, the engineered guide comprises a polynucleotide having at least 99% identity, at least 95% identity, at least 90% identity, at least 85% identity, at least 80% identity, or at least 70% identity to any one of SEQ ID NO: 2-SEQ ID NO: 395 or SEQ ID NO: 398-SEQ ID NO: 472. In some examples, the engineered guide comprises a polynucleotide having at least 99% length, at least 95% length, at least 90% length, at least 85% length, at least 80% length, or at least 70% length to the above SEQ ID NO: 2-SEQ ID NO: 395 or SEQ ID NO: 398-SEQ ID NO: 472. Further, a guide RNA targeting LRRK2 can comprise a sequence having a portion of any one of SEQ ID NO: 2-SEQ ID NO: 395 or SEQ ID NO: 398-SEQ ID NO: 472. In some examples, the engineered guide comprises a polynucleotide having a portion of any one of SEQ ID NO: 2-SEQ ID NO: 395 or SEQ ID NO: 398-SEQ ID NO: 472. In some examples, the engineered guide comprises a polynucleotide having a portion of any one of SEQ ID NO: 5, SEQ ID NO: 23, SEQ ID NO: 32, SEQ ID NO: 38, or SEQ ID NO: 50. In some examples, the engineered guide comprises a polynucleotide having a portion of SEQ ID NO: 5. In some examples, the engineered guide comprises a polynucleotide having a portion of SEQ ID NO: 23. In some examples, the engineered guide comprises a polynucleotide having a portion of SEQ ID NO: 32. In some examples, the engineered guide comprises a polynucleotide having a portion of SEQ ID NO: 38. In some examples, the engineered guide comprises a polynucleotide having a portion of SEQ ID NO: 50. In some examples, the engineered guide comprises a polynucleotide having at least 20-50 contiguous nucleotides form a portion of any one of SEQ ID NO: 2-SEQ ID NO: 395 or SEQ ID NO: 398-SEQ ID NO: 472. In some examples, the engineered guide comprises a polynucleotide having at least 20-50 contiguous nucleotides form a portion of any one of SEQ ID NO: 5, SEQ ID NO: 23, SEQ ID NO: 32, SEQ ID NO: 38, or SEQ ID NO: 50. In some examples, the engineered guide comprises a polynucleotide having from 20-50 contiguous nucleotides form a portion of SEQ ID NO: 5. In some examples, the engineered guide comprises a polynucleotide having from 20-50 contiguous nucleotides form a portion of SEQ ID NO: 23. In some examples, the engineered guide comprises a polynucleotide having from 20-50 contiguous nucleotides form a portion of SEQ ID NO: 32. In some examples, the engineered guide comprises a polynucleotide having from 20-50 contiguous nucleotides form a portion of SEQ ID NO: 38. In some examples, the engineered guide comprises a polynucleotide having from 20-50 contiguous nucleotides form a portion of SEQ ID NO: 50. In some examples, the engineered guide comprises a polynucleotide having 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 contiguous nucleotides form a portion of any one of SEQ ID NO: 2-SEQ ID NO: 395 or SEQ ID NO: 398-SEQ ID NO: 472. In some examples, the engineered guide comprises a polynucleotide having 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 contiguous nucleotides form a portion of any one of SEQ ID NO: 5, SEQ ID NO: 23, SEQ ID NO: 32, SEQ ID NO: 38, or SEQ ID NO: 50. In some examples, the engineered guide comprises a polynucleotide having 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 contiguous nucleotides form a portion of SEQ ID NO: 5. In some examples, the engineered guide comprises a polynucleotide having 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 contiguous nucleotides form a portion of SEQ ID NO: 23. In some examples, the engineered guide comprises a polynucleotide having 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 contiguous nucleotides form a portion of SEQ ID NO: 32. In some examples, the engineered guide comprises a polynucleotide having 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 contiguous nucleotides form a portion of SEQ ID NO: 38. In some examples, the engineered guide comprises a polynucleotide having 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 contiguous nucleotides form a portion of SEQ ID NO: 50. Said engineered guide RNA can be delivered via viral vector (e.g., encoded for and delivered via AAV) as disclosed herein and can be administered via any route of administration disclosed herein to a subject in need thereof. The subject may be human and may be at risk of developing or has developed a disease or condition associated with LRRK2 (e.g. Parkinson's disease or Crohn's disease). Such disease or condition can be at least partially caused by a mutation of LRRK2, for which an engineered guide RNA described herein can facilitate editing in, thus correcting the mutation in LRRK2 and reducing the incidence of the disease or condition in the subject. Thus, the guide RNAs of the present disclosure can be used in a method of treatment of diseases such as Crohn's disease or Parkinson's disease.

### Pharmaceutical Compositions

An engineered guide RNA targeting LRRK2 described herein (e.g., a guide RNA having a polynucleotide sequence of any one of SEQ ID NO: 2-SEQ ID NO: 395 or SEQ ID NO: 398-SEQ ID NO: 472) or a polynucleotide encoding an engineered guide RNA described herein can be formulated with a pharmaceutically acceptable carrier for administration to a subject (e.g., a human or a non-human animal). A pharmaceutically acceptable carrier can include, but is not limited to, phosphate buffered saline solution, water, emulsions (e.g., an oil/water emulsion or a water/oil emulsions), glycerol, liquid polyethylene glycols, aprotic solvents such (e.g., dimethylsulfoxide, N-methylpyrrolidone, or mixtures thereof), and various types of wetting agents, solubilizing agents, anti-oxidants, bulking agents, protein carriers such as albumins, any and all solvents, dispersion media, coatings, sodium lauryl sulfate, isotonic and absorption delaying agents, disintegrants (e.g., potato starch or sodium starch glycolate), and the like. The compositions also can include stabilizers and preservatives. Additional examples of carriers, stabilizers and adjuvants consistent with the compositions of the present disclosure can be found in, for example, Remington's Pharmaceutical Sciences. 21st Ed., Mack Publ. Co., Easton, Pa. (2005), incorporated herein by reference in its entirety.

### Delivery

An engineered guide RNA targeting LRRK2 described herein (e.g., a guide RNA having a polynucleotide sequence of any one of SEQ ID NO: 2-SEQ ID NO: 395 or SEQ ID NO: 398-SEQ ID NO: 472) or a polynucleotide encoding an engineered guide RNA described herein can be delivered via a delivery vehicle. In some embodiments, the delivery vehicle is a vector. A vector can facilitate delivery of the engineered guide RNA into a cell to genetically modify the cell. In some examples, the vector comprises DNA, such as double stranded or single stranded DNA. In some examples, the delivery vector can be a eukaryotic vector, a prokaryotic vector (e.g., a bacterial vector or plasmid), a viral vector, or any combination thereof. In some embodiments, the vector is an expression cassette. In some embodiments, a viral vector comprises a viral capsid, an inverted terminal repeat sequence, and the engineered polynucleotide can be used to deliver the engineered guide RNA to a cell.

In some embodiments, the viral vector can be a retroviral vector, an adenoviral vector, an adeno-associated viral (AAV) vector, an alphavirus vector, a lentivirus vector (e.g., human or porcine), a Herpes virus vector, an Epstein-Barr virus vector, an SV40 virus vectors, a pox virus vector, or a combination thereof. In some embodiments, the viral vector can be a recombinant vector, a hybrid vector, a chimeric vector, a self-complementary vector, a single-stranded vector, or any combination thereof.

In some embodiments, the viral vector can be an adeno-associated virus (AAV). In some embodiments, the AAV can be any AAV known in the art. In some embodiments, the viral vector can be of a specific serotype. In some embodiments, the viral vector can be an AAV1 serotype, AAV2 serotype, AAV3 serotype, AAV4 serotype, AAV5 serotype, AAV6 serotype, AAV7 serotype, AAV8 serotype, AAV9 serotype, AAV10 serotype, AAV11 serotype, AAV12 serotype, AAV13 serotype, AAV14 serotype, AAV15 serotype, AAV16 serotype, AAV.rh8 serotype, AAV.rh10 serotype, AAV.rh20 serotype, AAV.rh39 serotype, AAV.Rh74 serotype, AAV.RHM4-1 serotype, AAV.hu37 serotype, AAV.Anc80 serotype, AAV.Anc80L65 serotype, AAV.7m8 serotype, AAV.PHP.B serotype, AAV2.5 serotype, AAV2tYF serotype, AAV3B serotype, AAV.LK03 serotype, AAV.HSC1 serotype, AAV.HSC2 serotype, AAV.HSC3 serotype, AAV.HSC4 serotype, AAV.HSC5 serotype, AAV.HSC6 serotype, AAV.HSC7 serotype, AAV.HSC8 serotype, AAV.HSC9 serotype, AAV.HSC10 serotype, AAV.HSC11 serotype, AAV.HSC12 serotype, AAV.HSC13 serotype, AAV.HSC14 serotype, AAV.HSC15 serotype, AAV.HSC16 serotype, and AAVhu68 serotype, a derivative of any of these serotypes, or any combination thereof.

In some embodiments, the AAV vector can be a recombinant vector, a hybrid AAV vector, a chimeric AAV vector, a self-complementary AAV (scAAV) vector, a single-stranded AAV, or any combination thereof.

In some embodiments, the AAV vector can be a recombinant AAV (rAAV) vector. Methods of producing recombinant AAV vectors can be known in the art and generally involve, in some cases, introducing into a producer cell line: (1) DNA necessary for AAV replication and synthesis of an AAV capsid, (b) one or more helper constructs comprising the viral functions missing from the AAV vector, (c) a helper virus, and (d) the plasmid construct containing the genome of the AAV vector, e.g., ITRs, promoter and engineered guide RNA sequences, etc. In some examples, the viral vectors described herein can be engineered through synthetic or other suitable means by references to published sequences, such as those that can be available in the literature. For example, the genomic and protein sequences of various serotypes of AAV, as well as the sequences of the native terminal repeats (TRs), Rep proteins, and capsid subunits can be known in the art and can be found in the literature or in public databases such as GenBank or Protein Data Bank (PDB).

In some examples, methods of producing delivery vectors herein comprising packaging an engineered guide RNA of the present disclosure or an engineered polynucleotide of the present disclosure (e.g., an engineered polynucleotide encoding for an engineered guide RNA) in an AAV vector. In some examples, methods of producing the delivery vectors described herein comprise, (a) introducing into a cell: (i) a polynucleotide comprising a promoter and an engineered guide RNA payload disclosed herein; and (ii) a viral genome comprising a Replication (Rep) gene and Capsid (Cap) gene that encodes a wild-type AAV capsid protein or modified version thereof; (b) expressing in the cell the wild-type AAV capsid protein or modified version thereof; (c) assembling an AAV particle; and (d) packaging the payload disclosed herein in the AAV particle, thereby generating an AAV delivery vector. In some examples, the recombinant vectors comprise one or more inverted terminal repeats and the inverted terminal repeats comprise a 5′ inverted terminal repeat, a 3′ inverted terminal repeat, and a mutated inverted terminal repeat. In some examples, the mutated terminal repeat lacks a terminal resolution site, thereby enabling formation of a self-complementary AAV.

In some examples, a hybrid AAV vector can be produced by transcapsidation, e.g., packaging an inverted terminal repeat (ITR) from a first serotype into a capsid of a second serotype, wherein the first and second serotypes can be not the same. In some examples, the Rep gene and ITR from a first AAV serotype (e.g., AAV2) can be used in a capsid from a second AAV serotype (e.g., AAV5 or AAV9), wherein the first and second AAV serotypes may not be the same. As a non-limiting example, a hybrid AAV serotype comprising the AAV2 ITRs and AAV9 capsid protein can be indicated AAV2/9. In some examples, the hybrid AAV delivery vector comprises an AAV2/1, AAV2/2, AAV 2/4, AAV2/5, AAV2/8, or AAV2/9 vector.

In some examples, the AAV vector can be a chimeric AAV vector. In some examples, the chimeric AAV vector comprises an exogenous amino acid or an amino acid substitution, or capsid proteins from two or more serotypes. In some examples, a chimeric AAV vector can be genetically engineered to increase transduction efficiency, selectivity, or a combination thereof.

In some examples, the AAV vector comprises a self-complementary AAV genome. Self-complementary AAV genomes can be generally known in the art and contain both DNA strands which can anneal together to form double-stranded DNA.

In some examples, the delivery vector can be a retroviral vector. In some examples, the retroviral vector can be a Moloney Murine Leukemia Virus vector, a spleen necrosis virus vector, or a vector derived from the Rous Sarcoma Virus, Harvey Sarcoma Virus, avian leukosis virus, human immunodeficiency virus, myeloproliferative sarcoma virus, or mammary tumor virus, or a combination thereof. In some examples, the retroviral vector can be transfected such that the majority of sequences coding for the structural genes of the virus (e.g., gag, pol, and env) can be deleted and replaced by the gene(s) of interest.

In some examples, the delivery vehicle can be a non-viral vector. In some examples, the delivery vehicle can be a plasmid. In some embodiments, the plasmid comprises DNA. In some examples, the plasmid comprises circular double-stranded DNA. In some examples, the plasmid can be linear. In some examples, the plasmid comprises one or more genes of interest and one or more regulatory elements. In some examples, the plasmid comprises a bacterial backbone containing an origin of replication and an antibiotic resistance gene or other selectable marker for plasmid amplification in bacteria. In some examples, the plasmid can be a minicircle plasmid. In some examples, the plasmid contains one or more genes that provide a selective marker to induce a target cell to retain the plasmid. In some examples, the plasmid can be formulated for delivery through injection by a needle carrying syringe. In some examples, the plasmid can be formulated for delivery via electroporation. In some examples, the plasmids can be engineered through synthetic or other suitable means known in the art. For example, in some cases, the genetic elements can be assembled by restriction digest of the desired genetic sequence from a donor plasmid or organism to produce ends of the DNA which can then be readily ligated to another genetic sequence.

In some embodiments, the vector containing the engineered guide RNA or the engineered polynucleotide is a non-viral vector system. In some embodiments, the non-viral vector system comprises cationic lipids, or polymers. For example, the non-viral vector system comprises can be a liposome or polymeric nanoparticle. In some embodiments, the engineered polynucleotide or a non-viral vector comprising the engineered guide RNA or engineered polynucleotide is delivered to a cell by hydrodynamic injection or ultrasound.

### Administration

Administration can refer to methods that can be used to enable the delivery of an engineered guide RNA targeting LRRK2 described herein (e.g., a guide RNA having a polynucleotide sequence of any one of SEQ ID NO: 2-SEQ ID NO: 395 or SEQ ID NO: 398-SEQ ID NO: 472) or a polynucleotide encoding an engineered guide RNA described herein to the desired site of biological action. For example, an engineered guide RNA can be comprised in a DNA construct, a viral vector, or both and be administered by intravenous administration. Administration disclosed herein to an area in need of treatment or therapy can be achieved by, for example, and not by way of limitation, oral administration, topical administration, intravenous administration, inhalation administration, or any combination thereof. In some embodiments, delivery can include inhalation, otic, buccal, conjunctival, dental, endocervical, endosinusial, endotracheal, enteral, epidural, extra-amniotic, extracorporeal, hemodialysis, infiltration, interstitial, intraabdominal, intraamniotic, intraarterial, intraarticular, intrabiliary, intrabronchial, intrabursal, intracardiac, intracartilaginous, intracaudal, intracavernous, intracavitary, intracerebroventricular, intracisternal, intracorneal, intracoronal, intracoronary, intracorpous cavernaosum, intradermal, intradiscal, intraductal, intraduodenal, intradural, intraepidermal, intraesophageal, intragastric, intragingival, intrahippocampal, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathoracic, intratubular, intratumor, intratympanic, intrauterine, intravascular, intravenous, intravenous bolus, intravenous drip, intravesical, intravitreal, iontophoresis, irrigation, laryngeal, nasal, nasogastric, ophthalmic, oral, oropharyngeal, parenteral, percutaneous, periarticular, peridural, perineural, periodontal, rectal, retrobulbar, subarachnoid, subconjunctival, subcutaneous, sublingual, submucosal, topical, transdermal, transmucosal, transplacental, transtracheal, transtympanic, ureteral, urethral, vaginal, infraorbital, intraparenchymal, intrathecal, intraventricular, stereotactic, or any combination thereof. Delivery can include parenteral administration (including intravenous, subcutaneous, intrathecal, intraperitoneal, intramuscular, intravascular or infusion), oral administration, inhalation administration, intraduodenal administration, rectal administration, or a combination thereof. Delivery can include direct application to the affected tissue or region of the body. In some cases, topical administration can comprise administering a lotion, a solution, an emulsion, a cream, a balm, an oil, a paste, a stick, an aerosol, a foam, a jelly, a foam, a mask, a pad, a powder, a solid, a tincture, a butter, a patch, a gel, a spray, a drip, a liquid formulation, an ointment to an external surface of a surface, such as a skin. Delivery can include a parenchymal injection, an intra-thecal injection, an intra-ventricular injection, or an intra-cisternal injection. A composition provided herein can be administered by any method. A method of administration can be by intra-arterial injection, intracisternal injection, intramuscular injection, intraparenchymal injection, intraperitoneal injection, intraspinal injection, intrathecal injection, intravenous injection, intraventricular injection, stereotactic injection, subcutaneous injection, epidural, or any combination thereof. Delivery can include parenteral administration (including intravenous, subcutaneous, intrathecal, intraperitoneal, intramuscular, intravascular or infusion administration). In some embodiments, delivery can comprise a nanoparticle, a liposome, an exosome, an extracellular vesicle, an implant, or a combination thereof. In some cases, delivery can be from a device. In some instances, delivery can be administered by a pump, an infusion pump, or a combination thereof. In some embodiments, delivery can be by an enema, an eye drop, a nasal spray, or any combination thereof. In some instances, a subject can administer the composition in the absence of supervision. In some instances, a subject can administer the composition under the supervision of a medical professional (e.g., a physician, nurse, physician's assistant, orderly, hospice worker, etc.). In some embodiments, a medical professional can administer the composition.

In some cases, administering can be oral ingestion. In some cases, delivery can be a capsule or a tablet. Oral ingestion delivery can comprise a tea, an elixir, a food, a drink, a beverage, a syrup, a liquid, a gel, a capsule, a tablet, an oil, a tincture, or any combination thereof. In some embodiments, a food can be a medical food. In some instances, a capsule can comprise hydroxymethylcellulose. In some embodiments, a capsule can comprise a gelatin, hydroxypropylmethyl cellulose, pullulan, or any combination thereof. In some cases, capsules can comprise a coating, for example, an enteric coating. In some embodiments, a capsule can comprise a vegetarian product or a vegan product such as a hypromellose capsule. In some embodiments, delivery can comprise inhalation by an inhaler, a diffuser, a nebulizer, a vaporizer, or a combination thereof.

In some embodiments, disclosed herein can be a method, comprising administering a composition disclosed herein to a subject (e.g., a human) in need thereof. In some instances, the method can treat or prevent a disease in the subject.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments are presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, the term “about” a number can refer to that number plus or minus 10% of that number.

As disclosed herein, a “bulge” refers to the structure substantially formed only upon formation of the guide-target RNA scaffold, where contiguous nucleotides in either the engineered guide RNA or the target RNA are not complementary to their positional counterparts on the opposite strand. A bulge can independently have from 0 to 4 contiguous nucleotides on the guide RNA side of the guide-target RNA scaffold and 1 to 4 contiguous nucleotides on the target RNA side of the guide-target RNA scaffold or a bulge can independently have from 0 to 4 nucleotides on the target RNA side of the guide-target RNA scaffold and 1 to 4 contiguous nucleotides on the guide RNA side of the guide-target RNA scaffold. However, a bulge, as used herein, does not refer to a structure where a single participating nucleotide of the engineered guide RNA and a single participating nucleotide of the target RNA do not base pair—a single participating nucleotide of the engineered guide RNA and a single participating nucleotide of the target RNA that do not base pair is referred to herein as a “mismatch.” Further, where the number of participating nucleotides on either the guide RNA side or the target RNA side exceeds 4, the resulting structure is no longer considered a bulge, but rather, is considered an “internal loop.” A “symmetrical bulge” refers to a bulge where the same number of nucleotides is present on each side of the bulge. An “asymmetrical bulge” refers to a bulge where a different number of nucleotides are present on each side of the bulge.

The term “complementary” or “complementarity” refers to the ability of a nucleic acid to form one or more bonds with a corresponding nucleic acid sequence by, for example, hydrogen bonding (e.g., traditional Watson-Crick), covalent bonding, or other similar methods. In Watson-Crick base pairing, a double hydrogen bond forms between nucleobases T and A, whereas a triple hydrogen bond forms between nucleobases C and G. For example, the sequence A-G-T can be complementary to the sequence T-C-A. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” can mean that all the contiguous residues of a nucleic acid sequence will hydrogen bond with the same number of contiguous residues in a second nucleic acid sequence. “Substantially complementary” as used herein can refer to a degree of complementarity that can be at least 60%, 65%, 70%, 75%, 80%, 85%, 90%, 95%, 97%, 98%, 99%, or 100% over a region of 10, 15, 20, 25, 30, 35, 40, 45, 50, or more nucleotides, or can refer to two nucleic acids that hybridize under stringent conditions (i.e., stringent hybridization conditions). Nucleic acids can include nonspecific sequences. As used herein, the term “nonspecific sequence” or “not specific” can refer to a nucleic acid sequence that contains a series of residues that can be not designed to be complementary to or can be only partially complementary to any other nucleic acid sequence.

The terms “determining,” “measuring,” “evaluating,” “assessing,” “assaying,” and “analyzing” can be used interchangeably herein to refer to forms of measurement. The terms include determining if an element may be present or not (for example, detection). These terms can include quantitative, qualitative or quantitative and qualitative determinations. Assessing can be relative or absolute. “Detecting the presence of” can include determining the amount of something present in addition to determining whether it may be present or absent depending on the context.

The term “encode,” as used herein, refers to an ability of a polynucleotide to provide information or instructions sequence sufficient to produce a corresponding gene expression product. In a non-limiting example, mRNA can encode for a polypeptide during translation, whereas DNA can encode for an mRNA molecule during transcription.

An “engineered latent guide RNA” refers to an engineered guide RNA that comprises a portion of sequence that, upon hybridization or only upon hybridization to a target RNA, substantially forms at least a portion of a structural feature, other than a single A/C mismatch feature at the target adenosine to be edited.

As used herein, the term “facilitates RNA editing” by an engineered guide RNA refers to the ability of the engineered guide RNA when associated with an RNA editing entity and a target RNA to provide a targeted edit of the target RNA by the RNA edited entity. In some instances, the engineered guide RNA can directly recruit or position/orient the RNA editing entity to the proper location for editing of the target RNA. In other instances, the engineered guide RNA when hybridized to the target RNA forms a guide-target RNA scaffold with one or more structural features as described herein, where the guide-target RNA scaffold with one or more structural features recruits or positions/orients the RNA editing entity to the proper location for editing of the target RNA.

A “guide-target RNA scaffold,” as disclosed herein, is the resulting double stranded RNA formed upon hybridization of a guide RNA, with latent structure, to a target RNA. A guide-target RNA scaffold has one or more structural features formed within the double stranded RNA duplex upon hybridization. For example, the guide-target RNA scaffold can have one or more structural features selected from a bulge, mismatch, internal loop, hairpin, or wobble base pair.

As disclosed herein, a “hairpin” includes an RNA duplex wherein a portion of a single RNA strand has folded in upon itself to form the RNA duplex. The portion of the single RNA strand folds upon itself due to having nucleotide sequences that base pair to each other, where the nucleotide sequences are separated by an intervening sequence that does not base pair with itself, thus forming a base-paired portion and non-base paired, intervening loop portion.

As used herein, the term percent “identity,” in the context of two or more nucleic acid or polypeptide sequences, can refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

For purposes herein, percent identity and sequence similarity can be performed using the BLAST algorithm, which is described in Altschul et al. (J. Mol. Biol. 215:403-410 (1990)). Software for performing BLAST analyses is publicly available through the National Center for Biotechnology Information.

As disclosed herein, an “internal loop” refers to the structure substantially formed only upon formation of the guide-target RNA scaffold, where nucleotides in either the engineered guide RNA or the target RNA are not complementary to their positional counterparts on the opposite strand and where one side of the internal loop, either on the target RNA side or the engineered guide RNA side of the guide-target RNA scaffold, has 5 nucleotides or more. Where the number of participating nucleotides on both the guide RNA side and the target RNA side drops below 5, the resulting structure is no longer considered an internal loop, but rather, is considered a “bulge” or a “mismatch,” depending on the size of the structural feature. A “symmetrical internal loop” is formed when the same number of nucleotides is present on each side of the internal loop. An “asymmetrical internal loop” is formed when a different number of nucleotides is present on each side of the internal loop.

“Latent structure” refers to a structural feature that substantially forms only upon hybridization of a guide RNA to a target RNA. For example, the sequence of a guide RNA provides one or more structural features, but these structural features substantially form only upon hybridization to the target RNA, and thus the one or more latent structural features manifest as structural features upon hybridization to the target RNA. Upon hybridization of the guide RNA to the target RNA, the structural feature is formed and the latent structure provided in the guide RNA is, thus, unmasked.

“Messenger RNA” or “mRNA” are RNA molecules comprising a sequence that encodes a polypeptide or protein. In general, RNA can be transcribed from DNA. In some cases, precursor mRNA containing non-protein coding regions in the sequence can be transcribed from DNA and then processed to remove all or a portion of the non-coding regions (introns) to produce mature mRNA. As used herein, the term “pre-mRNA” can refer to the RNA molecule transcribed from DNA before undergoing processing to remove the non-protein coding regions.

As disclosed herein, a “mismatch” refers to a single nucleotide in a guide RNA that is unpaired to an opposing single nucleotide in a target RNA within the guide-target RNA scaffold. A mismatch can comprise any two single nucleotides that do not base pair. Where the number of participating nucleotides on the guide RNA side and the target RNA side exceeds 1, the resulting structure is no longer considered a mismatch, but rather, is considered a “bulge” or an “internal loop,” depending on the size of the structural feature.

As used herein, the term “polynucleotide” can refer to a single or double-stranded polymer of deoxyribonucleotide (DNA) or ribonucleotide (RNA) bases read from the 5′ to the 3′ end. The term “RNA” is inclusive of dsRNA (double stranded RNA), snRNA (small nuclear RNA), lncRNA (long non-coding RNA), mRNA (messenger RNA), miRNA (microRNA) RNAi (inhibitory RNA), siRNA (small interfering RNA), shRNA (short hairpin RNA), tRNA (transfer RNA), rRNA (ribosomal RNA), snoRNA (small nucleolar RNA), and cRNA (complementary RNA). The term DNA is inclusive of cDNA, genomic DNA, and DNA-RNA hybrids.

The term “protein”, “peptide” and “polypeptide” can be used interchangeably and in their broadest sense can refer to a compound of two or more subunit amino acids, amino acid analogs or peptidomimetics. The subunits can be linked by peptide bonds. In another embodiment, the subunit can be linked by other bonds, e.g., ester, ether, etc. A protein or peptide can contain at least two amino acids and no limitation can be placed on the maximum number of amino acids which can comprise a protein's or peptide's sequence. As used herein the term “amino acid” can refer to either natural amino acids, unnatural amino acids, or synthetic amino acids, including glycine and both the D and L optical isomers, amino acid analogs and peptidomimetics. As used herein, the term “fusion protein” can refer to a protein comprised of domains from more than one naturally occurring or recombinantly produced protein, where generally each domain serves a different function. In this regard, the term “linker” can refer to a protein fragment that can be used to link these domains together—optionally to preserve the conformation of the fused protein domains, prevent unfavorable interactions between the fused protein domains which can compromise their respective functions, or both.

The term “structured motif” refers to a combination of two or more structural features in a guide-target RNA scaffold.

The terms “subject,” “individual,” or “patient” can be used interchangeably herein. A “subject” refers to a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject can be diagnosed or suspected of being at high risk for a disease. In some cases, the subject may be not necessarily diagnosed or suspected of being at high risk for the disease

The term “in vivo” refers to an event that takes place in a subject's body.

The term “ex vivo” refers to an event that takes place outside of a subject's body. An ex vivo assay may be not performed on a subject. Rather, it can be performed upon a sample separate from a subject. An example of an ex vivo assay performed on a sample can be an “in vitro” assay.

The term “in vitro” refers to an event that takes places contained in a container for holding laboratory reagent such that it can be separated from the biological source from which the material can be obtained. In vitro assays can encompass cell-based assays in which living or dead cells can be employed. In vitro assays can also encompass a cell-free assay in which no intact cells can be employed.

The term “wobble base pair” refers to two bases that weakly pair. For example, a wobble base pair can refer to a G paired with a U.

The term “substantially forms” as described herein, when referring to a particular secondary structure, refers to formation of at least 80% of the structure under physiological conditions (e.g. physiological pH, physiological temperature, physiological salt concentration, etc.).

As used herein, the terms “treatment” or“treating” can be used in reference to a pharmaceutical or other intervention regimen for obtaining beneficial or desired results in the recipient. Beneficial or desired results include but can be not limited to a therapeutic benefit and/or a prophylactic benefit. A therapeutic benefit can refer to eradication or amelioration of one or more symptoms of an underlying disorder being treated. Also, a therapeutic benefit can be achieved with the eradication or amelioration of one or more of the physiological symptoms associated with the underlying disorder such that an improvement can be observed in the subject, notwithstanding that the subject can still be afflicted with the underlying disorder. A prophylactic effect includes delaying, preventing, or eliminating the appearance of a disease or condition, delaying or eliminating the onset of one or more symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease can undergo treatment, even though a diagnosis of this disease may not have been made.

### EXAMPLES

The following illustrative examples are representative of embodiments of the stimulation, systems, and methods described herein and are not meant to be limiting in any way.

### Example 1

High Throughput Screening of Engineered Guide RNAs Targeting LRRK2 mRNA

Using the compositions and methods described herein, high throughput screening (HTS) of long engineered guide RNAs (e.g., 100mer and longer) that target LRRK2 mRNA was performed, where said engineered guide RNAs form a micro-footprint comprised of various structural features in the guide-target RNA scaffold and form a barbell macro-footprint comprising two 6/6 internal loops near both ends of the guide-target RNA scaffold. Additionally, in this high throughput screen, self-annealing RNA structures were of a size (231 nucleotides) that allowed for screening for engineered guide RNAs that were 113 nucleotides in length, with the target adenosine to be edited positioned at the 57^{th} nucleotide. The high throughput screen was able to identify engineered guide RNAs that show high on-target adenosine editing (>60%, 30 min incubation with ADAR1 and ADAR2) and reduced to no local off-target adenosine editing (e.g., at the −2 position relative to the target adenosine to be edited, which is at position 0). Self-annealing RNA structures that formed a barbell macro-footprint in the guide-target RNA scaffold were screened to include 4 different micro-footprints (A/C mismatch (ATCTACAGCAGTACTGAGCAATGCCGTAGTCAGCAATCTTTGCA (SEQ ID NO: 102)), 2108 (ATTCTACGGCGGTACTGACCAATCCCGTAGTAGCAATCTTTGCA (SEQ ID NO: 103), 871 (ATTCTACAGTAGGACTGAGCACTGCCGAGCTGGGCAATCTTTGCA (SEQ ID NO: 104)), and 919 (CTTCTACAGCAGTTCGGAGGAATCCCGAGGTCAGCAATC TTTGCA (SEQ ID NO: 105)), tiling the position of the barbell macro-footprint from the −22 position to the −12 position at one end of the self-annealing RNA structure and from the +12 position to the +34 position at the other end of the self-annealing RNA structure. Self-annealing RNA structures comprising 1939 distinct guide RNA sequences and the sequences of the regions targeted by the guide RNAs were contacted with an RNA editing entity (e.g., a recombinant ADAR1 and/or ADAR2) for 30 minutes under conditions that allow for the editing of the regions targeted by the guide RNAs. The regions targeted by the guide RNAs were subsequently assessed for editing using next generation sequencing (NGS).

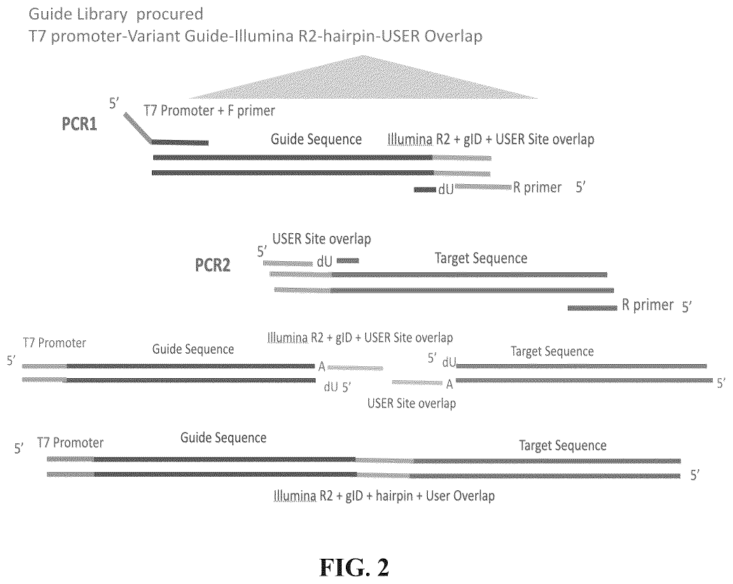

Libraries for screening of these longer engineered guides were generated as follows, and as summarized in FIG. 2: a candidate engineered guide library was procured having a construct with a T7 promoter, followed by the candidate engineered guide RNA sequence to be tested, followed by an Illumina R2 hairpin, followed by a sequence for a USER (Uracil-specific excision reagent) site Overlap. This library and the target sequence were PCR amplified, incorporating a deoxy-Uridine (dU) at the 3′ end of the constructs containing the candidate engineered guide RNA sequences and at the 5′ end of the target. Next, the PCR amplified library and target are incubated with the USER enzyme, resulting in nicking at the dU positions and ligation (using Taq ligase) of a given library construct containing the candidate engineered guide RNA sequence to the target sequence.

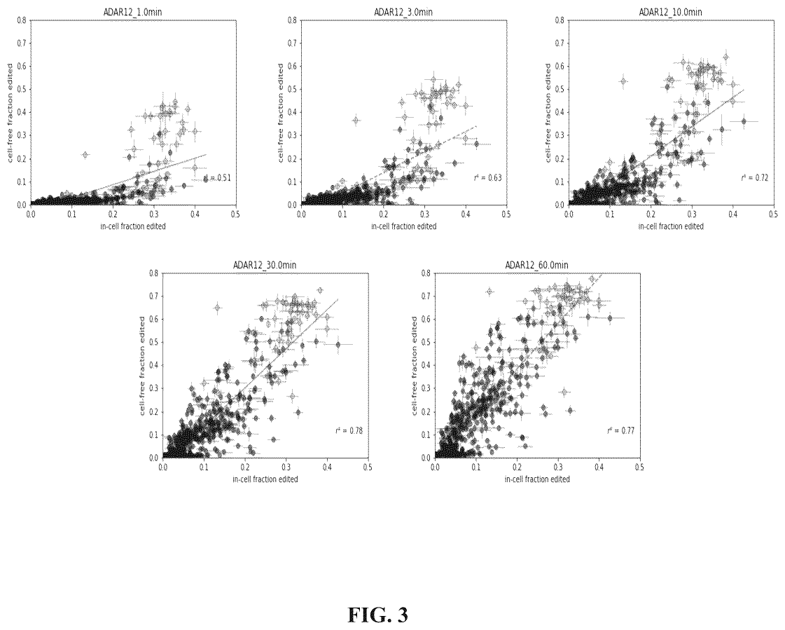

FIG. 3 shows a comparison of cell-free RNA editing using the methods and compositions described here versus in-cell RNA editing facilitated via the same engineered guide RNA sequence at various timepoints (20 s, 1 min, 3 min, 10 min, 30 min, and 60 min). In this experiment, 40 candidate guide RNAs were screened. 50 nM ADAR1+100 nM ADAR2 was present in each cell. The editing values for each guide and the position of the adenosine that was edited is presented in FIG. 3 as a cumulative of 6 values. The open circles represent on target adenosine editing for each guide, while the black circles represent editing of adenosines other than the on target adenosine (off target adenosines). As a whole, these data show that the cell-free high throughput screen is able to correlate well with in-cell RNA editing, in particular at certain timepoints (e.g., at 30 minutes).

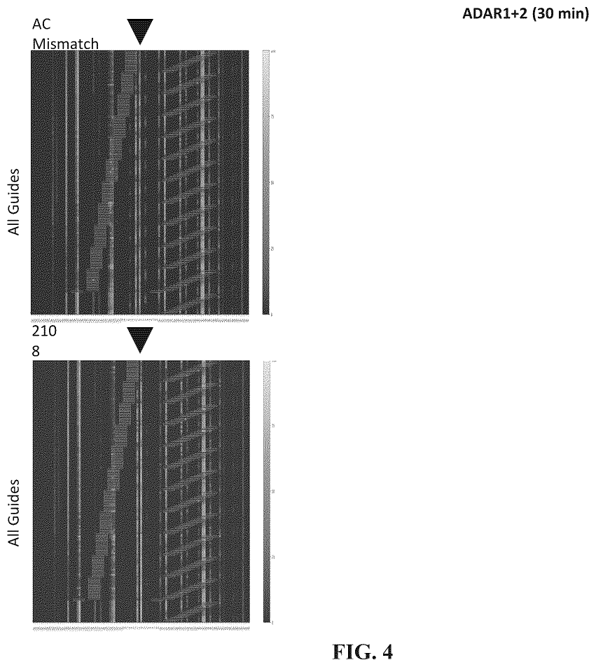

FIG. 4 shows heatmaps of all self-annealing RNA structures tested for the 4 micro-footprints described above formed within varying placement of a barbell macro-footprint. The y-axis shows all engineered guide RNAs tested and the x-axis shows the target sequence positions, with position 0 representing the target adenosine to be edited.

Exemplary engineered guide RNAs from the high throughput screen of this example are described in TABLE 2. The candidate engineered guide RNAs of TABLE 2 showed specific editing of the A nucleotide at position 6055 of the mRNA encoding the LRRK2 G2019S. Percent on-target editing is calculated by the following formula: the number of reads containing “G” at the target/the total number of reads. Specificity is calculated by the following formula: (percent on target editing+100)/(sum of off target editing percentage at selected off-targets sites+100). The addition of barbells produced specific editing patterns. In particular, the presence of barbell at position −14 and position +26 appeared to increase the specificity of ADAR editing. Thus, specificity can be improved significantly through the combination of micro-footprint structural features and macro-footprint structural features such as barbells.

TABLE 2
EXEMPLARY GUIDE RNAS THAT TARGET LRRK2 MRNA

| SEQ | | | Structural | |
|---|---|---|---|---|
| ID | | | Features | |
| NO | ID | Sequence | (target/guide) | Metrics |
| SEQ | LRRK2_bnPCR_ | CCCTGGTGTGCCCTCTGATGTTT | 1/1 U/G wobble | ADAR1 on target: |
| ID | ID94725_count351_ | TTATCCCCATTCTACAGCGGTAC | base pair at −3 | 0.38% |
| NO: | NoLoops_gID | TGAGCAAATCCGTGGTCAGCAA | position; | ADAR1 specificity: |
| 2 | 02513 | TCTTTGCAATGATGGCAGCATTG | 1/1 A/C mismatch | 0.5 |
| | | GGATACAGTGTGAAGAGCAGCA | at 0 position; | ADAR2 on target: |
| | | | 2_2/2 symmetric | 0.64% |
| | | | bulge at position | ADAR2 specificity: |
| | | | +2 (CA-AU); | 0.37 |
| | | | 1/1 U-G wobble | ADAR1/2 on target: |
| | | | base pair at +15 | 0.6% |
| | | | | ADAR1/2 specificity: |
| | | | | 0.43 |
| SEQ | LRRK2_bnPCR_ | CCCTGGTGTGCCCTCTGATGTTT | 6/6_ symmetric | ADAR1 on target: |
| ID | ID94725_count351_ | TTTAGGGGATTCTACAGCGGTA | internal loop at - | 0.32% |
| NO: | −14_26_gID_08570 | CTGAGCAAATCCGTGGTCAGCA | 14_position | ADAR1 specificity: |
| 3 | | ATCAAACGTATGATGGCAGCAT | (UGCAAA- | 0.77 |
| | | TGGGATACAGTGTGAAGAGCAG | AAACGU); | ADAR2 on target: |
| | | CA | 1/1 U/G wobble | 0.7% |
| | | | base pair at −3 | ADAR2 specificity: |
| | | | position; | 0.72 |
| | | | 1/1 A/C mismatch | ADAR1/2 on target: |
| | | | at 0 position; | 0.64% |
| | | | 2/2 CA/AU | ADAR1/2 specificity: |
| | | | symmetric bulge | 0.74 |
| | | | at +2_position; | |
| | | | 1/1 U/G wobble at | |
| | | | +15 position; | |
| | | | 6/6 symmetric | |
| | | | internal loop at | |
| | | | +26 position | |
| | | | (GGGGAU- | |
| | | | UAGGGG) | |
| SEQ | LRRK2_bnPCR_ | CCCTGGTGTGCCCTCTGATGTTT | 1/1 C/C mismatch | ADAR1 on target: |
| ID | ID79791_count610_ | TTATCCCCATTCTACATCTGTAG | at −4 position; | 0.43% |
| NO: | NoLoops_ | TGAGCAATTCCGTGCTCAGCAA | 1/1 (U/G) wobble | ADAR1 specificity: |
| 4 | gID_06724 | TCTTTGCAATGATGGCAGCATTG | base pair at −3 | 0.54 |
| | | GGATACAGTGTGAAGAGCAGCA | position; | ADAR2 on target: |
| | | | 1/1 A/C mismatch | 0.69% |
| | | | at 0 position; | ADAR2 specificity: |
| | | | 1/1 C/U mismatch | 0.38 |
| | | | at +2 position; 1/1 | ADAR1/2 on target: |
| | | | G/G mismatch at | 0.66% |
| | | | +11 position; 1/1 | ADAR1/2 specificity: |
| | | | U/U mismatch at | 0.42 |
| | | | +15 position; 1/1 | |
| | | | C/U mismatch at | |
| | | | +17 position | |
| SEQ | LRRK2_bnPCR_ | CCCTGGTGTGCCCTCTGATGTTT | 6/6 symmetric | ADAR1 on target: |
| ID | ID79791_count610_ | TTTAGGGGATTCTACATCTGTAG | internal loop at | 0.21% |
| NO: | −14_26_gID_09469 | TGAGCAATTCCGTGCTCAGCAA | −14 position | ADAR1 specificity: |
| 5 | | TCAAACGTATGATGGCAGCATT | (UGCAAA- | 0.77 |
| | | GGGATACAGTGTGAAGAGCAGC | AAACGU); | ADAR2 on target: |
| | | A | 1/1 C/C mismatch | 0.66% |
| | | | at −4 position; | ADAR2 specificity: |
| | | | 1/1 U/G wobble | 0.71 |
| | | | base pair at −3 | ADAR1/2 on target: |
| | | | position; | 0.62% |
| | | | 1/1 A/C mismatch | ADAR1/2 specificity: |
| | | | at 0 position; | 0.75 |
| | | | 1/1 C/U mismatch | |
| | | | at +2 position; | |
| | | | 1/1 G/G mismatch | |
| | | | at +11 position; | |
| | | | 1/1 U/U mismatch | |
| | | | +15 position; | |
| | | | 1/1 C/U mismatch | |
| | | | at +17 position; | |
| | | | 6/6 symmetric | |
| | | | internal loop at | |
| | | | +26 position | |
| | | | (GGGGAU- | |
| | | | UAGGGG) | |
| SEQ | LRRK2_bnPCR_ | CCCTGGTGTGCCCTCTGATGTTT | 1/1 C/C mismatch | ADAR1 on target: |
| ID | ID66010_count1326_ | TTATCCCCATTCTACACCTGGAC | at −4 position; | 0.45% |
| NO | NoLoops_ | TGAGAAATCCCGTACTCAGCAA | 1/1 A/C mismatch | ADAR1 specificity: |
| 6 | gID_09247 | TCTTTGCAATGATGGCAGCATTG | at 0 position; | 0.57 |
| | | GGATACAGTGTGAAGAGCAGCA | 1/1 C/C mismatch | ADAR2 on target: |
| | | | at +2 position; | 0.67% |
| | | | 1/1 G/A mismatch | ADAR2 specificity: |
| | | | at +6 position; | 0.39 |
| | | | 3/3 symmetric | ADAR1/2 on target: |
| | | | bulge at +13 | 0.65% |
| | | | position (ACU- | ADAR1/2 specificity: |
| | | | UGG); | 0.42 |
| | | | 1/1 C/C mismatch | |
| | | | at +17 position | |
| SEQ | LRRK2_bnPCR_ | CCCTGGTGTGCCCTCTGATGTTT | 6/6 symmetric | ADAR1 on target: |
| ID | ID66010_count1326_ | TTTAGGGGATTCTACACCTGGAC | internal loop at | 0.29% |
| NO: | −14 26_gID_11327 | TGAGAAATCCCGTACTCAGCAA | −14 position | ADAR1 specificity: |
| 7 | | TCAAACGTATGATGGCAGCATT | (UGCAAA- | 0.8 |
| | | GGGATACAGTGTGAAGAGCAGC | AAACGU); | ADAR2 on target: |
| | | A | 1/1 C/C mismatch | 0.64% |
| | | | at −4 position; | ADAR2 specificity: |
| | | | 1/1 A/C mismatch | 0.71 |
| | | | at 0 position; | ADAR1/2 on target: |
| | | | 1/1 C/C mismatch | 0.62% |
| | | | at +2 position; | ADAR1/2 specificity: |
| | | | 1/1 G/A mismatch | 0.76 |
| | | | at +6 position; | |
| | | | 3/3 symmetric | |
| | | | bulge at +13 | |
| | | | position (ACU- | |
| | | | UGG); | |
| | | | 1/1 C/C mismatch | |
| | | | at +17 position; | |
| | | | 6/6_symmetric | |
| | | | internal loop at | |
| | | | +26 position | |
| | | | (GGGGAU- | |
| | | | UAGGGG) | |
| SEQ | LRRK2_bnPCR_ | CCCTGGTGTGCCCTCTGATGTTT | 1/1 U/G wobble | ADAR1 on target: |
| ID | ID50437_count708_ | TTATCCCCATTCTACAGCAGTAC | base pair at −3 | 0.5% |
| NO: | NoLoops_ | GGTGCAGTGCCGTGGTCAGCAA | position; | ADAR1 specificity: |
| 8 | gID_11520 | TCTTTGCAATGATGGCAGCATTG | 1/1 A/C mismatch | 0.52 |
| | | GGATACAGTGTGAAGAGCAGCA | at 0 position; | ADAR2 on target: |
| | | | 1/1 U/G wobble | 0.7% |
| | | | base pair at +4 | ADAR2 specificity: |
| | | | position; | 0.38 |
| | | | 1/1 U/U mismatch | ADAR1/2 on target: |
| | | | at +8 position; | 0.65% |
| | | | 1/1 A/G mismatch | ADAR1/2 specificity: |
| | | | at +10 position | 0.43 |
| SEQ | LRRK2_bnPCR_ | CCCTGGTGTGCCCTCTGATGTTT | 6/6 symmetric | ADAR1 on target: |
| ID | ID50437_count708_ | TTTAGGGGATTCTACAGCAGTA | internal loop at | 0.36% |
| NO: | −14_26_gID_09857 | CGGTGCAGTGCCGTGGTCAGCA | −14 position | ADAR1 specificity: |
| 9 | | ATCAAACGTATGATGGCAGCAT | (UGCAAA- | 0.76 |
| | | TGGGATACAGTGTGAAGAGCAG | AAACGU); | ADAR2 |
| | | CA | 1/1 U/G wobble at | on target: |
| | | | −3 position; | 0.68% |
| | | | 1/1 A/C mismatch | ADAR2 specificity: |
| | | | at 0 position; | 0.68 |
| | | | 1/1 U/G wobble | |
| | | | base pair at +4 | ADAR1/2 on target: |
| | | | position; | 0.69% |
| | | | 1/1 U/U mismatch | ADAR1/2 specificity: |
| | | | at +8 position; | 0.71 |
| | | | 1/1 A/G mismatch | |
| | | | at +10 position; | |
| | | | 6/6 symmetric | |
| | | | internal loop at | |
| | | | +26 position | |
| | | | (GGGGAU- | |
| | | | UAGGGG) | |
| SEQ | LRRK2_bnPCR_ | CCCTGGTGTGCCCTCTGATGTTT | 1/1 U/G wobble | ADAR1 on target: |
| ID | ID41799_count730_ | TTATCCCCCTTCTACAGCAGTAG | base pair at −3 | 0.4% |
| NO: | _NoLoops_ | TGAGTTTTGCCGTGGTCAGCAAT | position; | ADAR1 specificity: |
| 10 | gID_04947 | CTTTGCAATGATGGCAGCATTG | 1/1 A/C mismatch | 0.56 |
| | | GGATACAGTGTGAAGAGCAGCA | at 0 position; | ADAR2 on target: |
| | | | 2/2 symmetric | 0.69% |
| | | | bulge at +4 | ADAR2 specificity: |
| | | | position (UU- | 0.37 |
| | | | UU); | ADAR1/2 on target: |
| | | | 1/1 G/U wobble | 0.66% |
| | | | base pair at +6 | ADAR1/2 specificity: |
| | | | position; | 0.41 |
| | | | 1/1 G/G mismatch | |
| | | | at +11 position; | |
| | | | 1/1 U/C mismatch | |
| | | | at +25 position | |
| SEQ | LRRK2_bnPCR_ | CCCTGGTGTGCCCTCTGATGTTT | 6/6 symmetric | ADAR1 on target: |
| ID | ID41799_count730_ | TTTAGGGGCTTCTACAGCAGTA | internal loop at | 0.3% |
| NO: | −14_26_gID_07755 | GTGAGTTTTGCCGTGGTCAGCA | −14_position | ADAR1 specificity: |
| 11 | | ATCAAACGTATGATGGCAGCAT | (UGCAAA- | 0.79 |
| | | TGGGATACAGTGTGAAGAGCAG | AAACGU); | ADAR2 on target: |
| | | CA | 1/1 U/G wobble | 0.68% |
| | | | base pair at −3 | ADAR2 specificity: |
| | | | position; | 0.67 |
| | | | 1/1 A/C mismatch | ADAR1/2 on target: |
| | | | at 0 position; | 0.65% |
| | | | 2/2 symmetric | ADAR1/2 specificity: |
| | | | bulge at +4 | 0.73 |
| | | | position (UU- | |
| | | | UU); | |
| | | | 1/1 G/U wobble | |
| | | | base pair at +6 | |
| | | | position; | |
| | | | 1/1 G/G mismatch | |
| | | | at +11 position; | |
| | | | 7/7 symmetric | |
| | | | internal loop at | |
| | | | +25 position | |
| | | | (UGGGGAU- | |
| | | | UAGGGGC) | |
| SEQ | LRRK2_bnPCR_ | CCCTGGTGTGCCCTCTGATGTTT | 1/1 C/A mismatch | ADAR1 on target: |
| ID | ID35277_count295_ | TTATCCCCATTCTACAGCAGTCC | at −4 position; | 0.45% |
| NO: | NoLoops_ | TGTACAGTGCCGTGATCAGCAA | 1/1 U/G wobble | ADAR1 specificity: |
| 12 | gID_06840 | TCTTTGCAATGATGGCAGCATTG | base pair at −3 | 0.57 |
| | | GGATACAGTGTGAAGAGCAGCA | position; | ADAR2 on target: |
| | | | 1/1 A/C mismatch | 0.66% |
| | | | at 0 position; | ADAR2 specificity: |
| | | | 1/1 U/G wobble | 0.39 |
| | | | base pair at +4 | ADAR1/2 on target: |
| | | | position; | 0.64% |
| | | | 2/2 symmetric | ADAR1/2 specificity: |
| | | | bulge at +7 | 0.44 |
| | | | position (CU- | |
| | | | UA); | |
| | | | 1/1 U/C mismatch | |
| | | | at +12 position | |
| SEQ | LRRK2_bnPCR_ | CCCTGGTGTGCCCTCTGATGTTT | 6/6 symmetric | ADAR1 on target: |
| ID | ID35277_count295_ | TTTAGGGGATTCTACAGCAGTCC | internal loop at | 0.27% |
| NO: | −14_26_gID_09359 | TGTACAGTGCCGTGATCAGCAA | −14 position | ADAR1 specificity: |
| 13 | | TCAAACGTATGATGGCAGCATT | (UGCAAA- | 0.76 |
| | | GGGATACAGTGTGAAGAGCAGC | AAACGU); | ADAR2 on target: |
| | | A | 1/1 C/A mismatch | 0.68% |
| | | | at −4 position; | ADAR2 specificity: |
| | | | 1/1 U/G wobble | 0.68 |
| | | | base pair at −3 | ADAR1/2 on target: |
| | | | position; | 0.65% |
| | | | 1/1 A/C mismatch | ADAR1/2 specificity: |
| | | | at 0 position; | 0.71 |
| | | | 1/1 U/G wobble | |
| | | | base pair at +4 | |
| | | | position; | |
| | | | 2/2 symmetric | |
| | | | bulge at +7 | |
| | | | position (CU- | |
| | | | UA); | |
| | | | 1/1 U/C mismatch | |
| | | | at +12 position; | |
| | | | 6/6 symmetric | |
| | | | internal loop at | |
| | | | +26 position | |
| | | | (GGGGAU- | |
| | | | UAGGGG | |
| SEQ | LRRK2_bnPCR_ | CCCTGGTGTGCCCTCTGATGTTT | 6/6 symmetric | ADAR1 on target: |
| ID | ID34601_count2108_ | TTATCCCCATTCGCCTGAGGTAC | internal loop at | 0.33% |
| NO | −10_16_gID_01444 | TGACCAATCCCGTAGTTAGCCTA | −10 position | ADAR1 specificity: |
| 14 | | GGCTGCAATGATGGCAGCATTG | (AAGAUU- | 0.7 |
| | | GGATACAGTGTGAAGAGCAGCA | CUAGGC); | ADAR2 on target: |
| | | | 1/1 G/U wobble | 0.63% |
| | | | base pair at −6 | ADAR2 specificity: |
| | | | position; | 0.52 |
| | | | 1/1 A/C mismatch | ADAR1/2 on target: |
| | | | at 0 position; | 0.6% |
| | | | 1/1 C/C mismatch | ADAR1/2 specificity: |
| | | | at +2 position; | 0.58 |
| | | | 1/1 C/C mismatch | |
| | | | at +7 position; | |
| | | | 1/1 U/G wobble | |
| | | | base pair at +15 | |
| | | | position; | |
| | | | 6/6 symmetric | |
| | | | internal loop at | |
| | | | +16 position | |
| | | | (GCUGUA- | |
| | | | GCCUGA) | |
| SEQ | LRRK2_bnPCR_ | CCCTGGTGTGCCCTCTGATGTTT | 6/6 symmetric | ADAR1 on target: |
| ID | ID33405_count860_ | TTCTACAGATTCTACAGCAGTAC | internal loop at | 0.22% |
| NO: | −22_26_gID_00318 | AGAGGACTGCCGAGGTCACCAA | −22 position | ADAR1 specificity: |
| 15 | | TCTTTGCAATAACCTGAGCATTG | (GCCAUC- | 0.73 |
| | | GGATACAGTGTGAAGAGCAGCA | AACCUG); | ADAR2 on target: |
| | | | 1/1 C/C mismatch | 0.61% |
| | | | at −8 position; | ADAR2 specificity: |
| | | | 1/1 U/G wobble | 0.56 |
| | | | base pair at −3 | ADAR1/2 on target: |
| | | | position; | 0.56% |
| | | | 1/1 A/A mismatch | ADAR1/2 specificity: |
| | | | at −2 position; | 0.63 |
| | | | 1/1 A/C mismatch | |
| | | | at 0 position; | |
| | | | 1/1 U/C mismatch | |
| | | | at +4 position; | |
| | | | 1/1 G/G mismatch | |
| | | | at +6 position; | |
| | | | 1/1 A/A mismatch | |
| | | | at +10 position; | |
| | | | 2/2 symmetric | |
| | | | bulge at +26 | |
| | | | position (GG- | |
| | | | AG); | |
| | | | 3/3 symmetric | |
| | | | bulge at +29 | |
| | | | position (GAU- | |
| | | | CUA) | |
| SEQ | LRRK2_bnPCR_ | CCCTGGTGTGCCCTCTGATGTTT | 6/6 symmetric | ADAR1 on target: |
| ID | ID33405_count860_ | TTTAGGGGATTCTACAGCAGTA | internal loop at | 0.22% |
| NO: | −20_26_gID_09192 | CAGAGGACTGCCGAGGTCACCA | −20 position | ADAR1 specificity: |
| 16 | | ATCTTTGCATACTACGCAGCATT | (CAUCAU- | 0.73 |
| | | GGGATACAGTGTGAAGAGCAGC | UACUAC); | ADAR2 on target: |
| | | A | 1/1 C/C mismatch | 0.63% |
| | | | at −8 position; | ADAR2 specificity: |
| | | | 1/1 U/G wobble | 0.58 |
| | | | base pair at −3 | ADAR1/2 on target: |
| | | | position; | 0.58% |
| | | | 1/1 A/A mismatch | ADAR1/2 specificity: |
| | | | at −2 position; | 0.64 |
| | | | 1/1 A/C mismatch | |
| | | | at 0 position; | |
| | | | 1/1 U/C mismatch | |
| | | | at +4 position; 1/1 | |
| | | | G/G mismatch at | |
| | | | +6 position; | |
| | | | 1/1 A/A mismatch | |
| | | | at +10 position; | |
| | | | 6/6 symmetric | |
| | | | internal loop at | |
| | | | +26 position | |
| | | | (GGGGAU- | |
| | | | UAGGGG) | |
| SEQ | LRRK2_bnPCR_ | CCCTGGTGTGCCCTCTGATGTTT | 6/6 symmetric | ADAR1 on target: |
| ID | ID33405_count860_ | TTCGGGAGATTCTACAGCAGTA | internal loop at | 0.21% |
| NO: | −16_26_gID_11898 | CAGAGGACTGCCGAGGTCACCA | −16 position | ADAR1 specificity: |
| 17 | | ATCTTGTCTAGGATGGCAGCATT | (AUUGCA- | 0.76 |
| | | GGGATACAGTGTGAAGAGCAGC | GUCUAG); | ADAR2 on target: |
| | | A | 1/1 C/C mismatch | 0.66% |
| | | | at −8 position; | ADAR2 specificity: |
| | | | 1/1 U/G wobble at | 0.6 |
| | | | −3 position; | ADAR1/2 on target: |
| | | | 1/1 A/A mismatch | 0.59% |
| | | | at −2 position; | ADAR1/2 specificity: |
| | | | 1/1 A/C mismatch | 0.63 |
| | | | at 0 position; | |
| | | | 1/1 U/C mismatch | |
| | | | at +4 position; | |
| | | | 1/1 G/G mismatch | |
| | | | at +6 position; | |
| | | | 1/1 A/A mismatch | |
| | | | at +10 position; | |
| | | | 6/6 symmetric | |
| | | | internal loop at | |
| | | | +26 position | |
| | | | (GGGGAU- | |
| | | | CGGGAG) | |
| SEQ | LRRK2_bnPCR_ | CCCTGGTGTGCCCTCTGATGTAT | 6/6 symmetric | ADAR1 on target: |
| ID | ID33405_count860_ | GTTCCCCCATTCTACAGCAGTAC | internal loop at | 0.39% |
| NO: | −12 30 gID_05698 | AGAGGACTGCCGAGGTCACCAA | −12 position | ADAR1 specificity: |
| 18 | | CGCTAACAATGATGGCAGCATT | (CAAAGA- | 0.75 |
| | | GGGATACAGTGTGAAGAGCAGC | CGCUAA); | ADAR2 on target: |
| | | A | 1/1 C/C mismatch | 0.45% |
| | | | at −8 position; | ADAR2 specificity: |
| | | | 1/1 U/G wobble | 0.55 |
| | | | base pair at −3 | ADAR1/2 on target: |
| | | | position; | 0.5% |
| | | | 1/1 A/A mismatch | ADAR1/2 specificity: |
| | | | at −2 position; | 0.66 |
| | | | 1/1 A/C mismatch | |
| | | | at 0 position; | |
| | | | 1/1 U/C mismatch | |
| | | | at +4 position; | |
| | | | 1/1 G/G mismatch | |
| | | | at +6 position; | |
| | | | 1/1 A/A mismatch | |
| | | | at +10 position; | |
| | | | 4/4 symmetric | |
| | | | bulge at +30 | |
| | | | position (AUAA- | |
| | | | GUUC); | |
| | | | 1/1 A/A mismatch | |
| | | | at +35 position | |
| SEQ | LRRK2_bnPCR_ | CCCTGGTGTGCCCTCTGATGTTT | 6/6 symmetric | ADAR1 on target: |
| ID | ID33405_count860_ | TTATGGAAAGTCTACAGCAGTA | internal loop at −8 | 0.33% |
| NO: | −8_24_gID_05952 | CAGAGGACTGCCGAGGTCACTT | position | ADAR1 specificity: |
| 19 | | TGATTTGCAATGATGGCAGCATT | (GAUUGC- | 0.67 |
| | | GGGATACAGTGTGAAGAGCAGC | CUUUGA); | ADAR2 on target: |
| | | A | 1/1 U/G wobble | 0.58% |
| | | | base pair at −3 | ADAR2 specificity: |
| | | | position; | 0.58 |
| | | | 1/1 A/A mismatch | ADAR1/2 on target: |
| | | | at −2 position; | 0.49% |
| | | | 1/1 A/C mismatch | ADAR1/2 specificity: |
| | | | at 0 position; | 0.64 |
| | | | 1/1 U/C mismatch | |
| | | | at +4 position; | |
| | | | 1/1 G/G mismatch | |
| | | | at +6 position; | |
| | | | 1/1 A/A mismatch | |
| | | | at +10 position; | |
| | | | 6/6 symmetric | |
| | | | internal loop at | |
| | | | +24 position | |
| | | | (AUGGGG- | |
| | | | GGAAAG) | |
| SEQ | LRRK2_bnPCR_ | CCCTGGTGTGCCCTCTGATGTTT | 6/6 symmetric | ADAR1 on target: |
| ID | ID29209_count871_ | TTCGAAGAATTCTACAGTAGGA | internal loop at | 0.25% |
| NO: | −22_26_gID_01244 | CTGAGCACTGCCGAGCTGGGCA | −22_ position | ADAR1 specificity: |
| 20 | | ATCTTTGCAATCAAAAAAGCAT | (GCCAUC- | 0.72 |
| | | TGGGATACAGTGTGAAGAGCAG | CAAAAA); | ADAR2 on target: |
| | | CA | 1/1 U/G wobble | 0.59% |
| | | | base pair at −7 | ADAR2 specificity: |
| | | | position; | 0.61 |
| | | | 1/1 G/G mismatch | ADAR1/2 on target: |
| | | | at −6 position; | 0.56% |
| | | | 0/1 asymmetric | ADAR1/2 specificity: |
| | | | bulge at −4 | 0.67 |
| | | | position (-C); | |
| | | | 1/0 asymmetric | |
| | | | bulge at −2 | |
| | | | position (A-); | |
| | | | 1/1 A/C mismatch | |
| | | | at 0 position; | |
| | | | 1/1 U/C mismatch | |
| | | | at +4 position; | |
| | | | 1/1 A/G mismatch | |
| | | | at +13 position; | |
| | | | 1/1 G/U wobble at | |
| | | | +16 position; | |
| | | | 6/6 symmetric | |
| | | | internal loop at | |
| | | | +26 position | |
| | | | (GGGGAU- | |
| | | | CGAAGA) | |
| SEQ | LRRK2_bnPCR_ | CCCTGGTGTGCCCTCTGATGTTT | 6/6 symmetric | ADAR1 on target: |
| ID | ID29209_count820_ | TTTAGGGGATTCTACAGTAGGA | internal loop at | 0.25% |
| NO: | −26_gID_07422 | CTGAGCACTGCCGAGCTGGGCA | −20 position | ADAR1 specificity: |
| 21 | | ATCTTTGCATACTACGCAGCATT | (CAUCAU- | 0.73 |
| | | GGGATACAGTGTGAAGAGCAGC | UACUAC); | ADAR2 on target: |
| | | A | 1/1 U/G wobble | 0.62% |
| | | | base pair at −7 | ADAR2 specificity: |
| | | | position; | 0.67 |
| | | | 1/1 G/G mismatch | ADAR1/2 on target: |
| | | | at −6 position; | 0.6% |
| | | | 0/1 asymmetric | ADAR1/2 specificity: |
| | | | bulge at −4 | 0.69 |
| | | | position (-C); | |
| | | | 1/0 asymmetric | |
| | | | bulge at −2 | |
| | | | position (A-); | |
| | | | 1/1 A/C mismatch | |
| | | | at 0 position; | |
| | | | 1/1 U/C mismatch | |
| | | | at +4 position; | |
| | | | 1/1 A/G mismatch | |
| | | | at +13 position; | |
| | | | 1/1 G/U wobble | |
| | | | base pair at +16 | |
| | | | position; | |
| | | | 6/6 symmetric | |
| | | | internal loop at | |
| | | | +26 position | |
| | | | (GGGGAU- | |
| | | | UAGGGG) | |
| SEQ | LRRK2_bnPCR_ | CCCTGGTGTGCCCTCTGATGTTT | 6/6 symmetric | ADAR1 on target: |
| ID | ID29209_count871_ | TTCTACAGATTCTACAGTAGGAC | internal loop at | 0.32% |
| NO: | −16_26_gID_01312 | TGAGCACTGCCGAGCTGGGCAA | −16 position | ADAR1 specificity: |
| 22 | | TCTTCATTTGGATGGCAGCATTG | (AUUGCA- | 0.77 |
| | | GGATACAGTGTGAAGAGCAGCA | CAUUUG); | ADAR2 on target: |
| | | | 1/1 U/G wobble | 0.6% |
| | | | base pair at −7 | ADAR2 specificity: |
| | | | position; | 0.66 |
| | | | 1/1 G/G mismatch | ADAR1/2 on target: |
| | | | at −6 position; | 0.57% |
| | | | 0/1 aysmmetric | ADAR1/2 specificity: |
| | | | bulge at −4 | 0.69 |
| | | | position (-C); | |
| | | | 1/0 asymmetric | |
| | | | bulge at −2 | |
| | | | position (A-); | |
| | | | 1/1 A/C mismatch | |
| | | | at 0 position; | |
| | | | 1/1 U/C mismatch | |
| | | | at +4 position; | |
| | | | 1/1 A/G mismatch | |
| | | | at +13 position; | |
| | | | 1/1 G/U wobble | |
| | | | base pair at +16 | |
| | | | position; | |
| | | | 2/2 symmetric | |
| | | | bulge at +26 | |
| | | | position (GG- | |
| | | | AG); | |
| | | | 3/3 symmetric | |
| | | | bulge at +29 | |
| | | | position (GAU- | |
| | | | CUA) | |
| SEQ | LRRK2_bnPCR_ | CCCTGGTGTGCCCTCTGATGTTT | 6/6 symmetric | ADAR1 on target: |
| ID | ID29209_count871_ | TTATGGGGTGTCTACAGTAGGA | internal loop at | 0.28% |
| NO: | −16_24_gID_00727 | CTGAGCACTGCCGAGCTGGGCA | −16 position | ADAR1 specificity: |
| 23 | | ATCTTGAGTCGGATGGCAGCAT | (AUUGCA- | 0.81 |
| | | TGGGATACAGTGTGAAGAGCAG | GAGUCG); | ADAR2 on target: |
| | | CA | 1/1 U/G wobble | 0.62% |
| | | | base pair at −7 | ADAR2 specificity: |
| | | | position; | 0.73 |
| | | | 1/1 G/G mismatch | ADAR1/2 on target: |
| | | | at −6 position; | 0.58% |
| | | | 0/1 asymmetric | ADAR1/2 specificity: |
| | | | bulge at −4 | 0.76 |
| | | | position (-C); | |
| | | | 1/0 asymmetric | |
| | | | bulge at −2 | |
| | | | position (A-); | |
| | | | 1/1 A/C mismatch | |
| | | | at 0 position; | |
| | | | 1/1 U/C mismatch | |
| | | | at +4 position; | |
| | | | 1/1 A/G mismatch | |
| | | | at +13 position; | |
| | | | 1/1 G/U wobble at | |
| | | | +16 position; 6/6 | |
| | | | symmetric internal | |
| | | | loop at +24 | |
| | | | position | |
| | | | (AUGGGG- | |
| | | | GGGGUG) | |
| SEQ | LRRK2_bnPCR_ | CCCTGGTGTGCCCTCTGATGTTT | 6/6 symmetric | ADAR1 on target: |
| ID | ID29209_count871_ | TTATCCGAAACATACAGTAGGA | internal loop at | 0.2% |
| NO: | −16_22_gID_11427 | CTGAGCACTGCCGAGCTGGGCA | −16 position | ADAR1 specificity: |
| 24 | | ATCTTCTCCCAGATGGCAGCATT | (AUUGCA- | 0.81 |
| | | GGGATACAGTGTGAAGAGCAGC | CUCCCA); | ADAR2 on target: |
| | | A | 1/1 U/G wobble at | 0.55% |
| | | | −7 position; | ADAR2 specificity: |
| | | | 1/1 G/G mismatch | 0.74 |
| | | | at −6 positon; | ADAR1/2 on target: |
| | | | 0/1 asymmetric | 0.48% |
| | | | bulge at −4 | ADAR1/2 specificity: |
| | | | position (-C); | 0.75 |
| | | | 1/0 asymmetric | |
| | | | bulge at −2 | |
| | | | position (A-); | |
| | | | 1/1 A/C mismatch | |
| | | | at 0 position; | |
| | | | 1/1 U/C mismatch | |
| | | | at +4 position; | |
| | | | 1/1 A/G mismatch | |
| | | | at +13 position; | |
| | | | 1/1 G/U wobble | |
| | | | base pair at +16 | |
| | | | position; | |
| | | | 6/6 symmetric | |
| | | | internal loop at | |
| | | | +22 position | |
| | | | (GAAUGG- | |
| | | | GAAACA) | |
| SEQ | LRRK2_bnPCR_ | CCCTGGTGTGCCCTCTGATGTTT | 6/6 symmetric | ADAR1 on target: |
| ID | ID29209_count871_− | TTCATGAGATTCTACAGTAGGA | internal loop at | 0.45% |
| NO: | −10_26_gID_00091 | CTGAGCACTGCCGAGCTGGGCC | −10 position | ADAR1 specificity: |
| 25 | | TCAGGTGCAATGATGGCAGCAT | (AAGAUU- | 0.78 |
| | | TGGGATACAGTGTGAAGAGCAG | CUCAGG); | ADAR2 on target: |
| | | CA | 1/1 U/G wobble | 0.64% |
| | | | base pair at −7 | ADAR2 specificity: |
| | | | position; | 0.7 |
| | | | 1/1 G/G mismatch | ADAR1/2 on target: |
| | | | at −6 position; | 0.59% |
| | | | 0/1 asymmetric | ADAR1/2 specificity: |
| | | | bulge at −4 | 0.73 |
| | | | position (-C); | |
| | | | 1/0 asymmetric | |
| | | | bulge at −2 | |
| | | | position (A-); | |
| | | | 1/1 A/C mismatch | |
| | | | at 0 position; | |
| | | | 1/1 U/C mismatch | |
| | | | at +4 position; | |
| | | | 1/1 A/G mismatch | |
| | | | at +13 position; | |
| | | | 1/1 G/U wobble | |
| | | | base pair at +16 | |
| | | | position; | |
| | | | 6/6 symmetric | |
| | | | internal loop at | |
| | | | +26 position | |
| | | | (GGGGAU- | |
| | | | CAUGAG) | |
| SEQ | LRRK2_bnPCR_ | CCCTGGTGTGCCCTCTGATGTTT | 4/4 symmetric | ADAR1 on target: |
| ID | ID29209_count871_ | TTTACAGAATTCTACAGTAGGA | bulge at -9 | 0.38% |
| NO: | −8 26 gID 11298 | CTGAGCACTGCCGAGCTGGGGT | position (AUUG- | ADAR1 specificity: |
| 26 | | TCCTTTGCAATGATGGCAGCATT | GUUC); | 0.74 |
| | | GGGATACAGTGTGAAGAGCAGC | 1/1 U/G wobble | ADAR2 on target: |
| | | A | base pair at −7 | 0.57% |
| | | | position; | ADAR2 specificity: |
| | | | 1/1 G/G mismatch | 0.65 |
| | | | at −6 position; | ADAR1/2 on target: |
| | | | 0/1 asymmetric | 0.53% |
| | | | bulge at −4 | ADAR1/2 specificity: |
| | | | position (-C); | 0.71 |
| | | | 1/0 asymmetric | |
| | | | bulge at −2 | |
| | | | position (A-); | |
| | | | 1/1 A/C mismatch | |
| | | | at 0 position; | |
| | | | 1/1 U/C mismatch | |
| | | | at +4 position; | |
| | | | 1/1 A/G mismatch | |
| | | | at +13 position; | |
| | | | 1/1 G/U wobble | |
| | | | base pair at +16 | |
| | | | position; | |
| | | | 6/6 symmetric | |
| | | | internal loop at | |
| | | | +26 position | |
| | | | (GGGGAU- | |
| | | | UACAGA) | |
| SEQ | LRRK2_bnPCR_ | CCCTGGTGTGCCCTCTGATGTTT | 6/6 symmetric | ADAR1 on target: |
| ID | ID28134_count1700_ | TTTAGGGGATTCTACCGCAGTAC | internal loop at | 0.39% |
| NO: | −20_26_gID_06283 | TACCCGATCCCGTAGTCAGCAA | −20 position | ADAR1 specificity: |
| 27 | | TCTTTGCATACTACGCAGCATTG | (CAUCAU- | 0.71 |
| | | GGATACAGTGTGAAGAGCAGCA | UACUAC); 1/1 | ADAR2 on target: |
| | | | A/C mismatch at 0 | 0.7% |
| | | | position; | ADAR2 specificity: |
| | | | 1/1 C/C mismatch | 0.64 |
| | | | at +2 position; | ADAR1/2 on target: |
| | | | 1/1 U/G wobble | 0.56% |
| | | | base pair at +5 | ADAR1/2 specificity: |
| | | | position; | 0.7 |
| | | | 3/3 symmetric | |
| | | | bulge at +7 | |
| | | | position (CUC- | |
| | | | ACC); | |
| | | | 1/1 U/C mismatch | |
| | | | at +18 position; | |
| | | | 6/6 symmetric | |
| | | | internal loop at | |
| | | | +26 position | |
| | | | (GGGGAU- | |
| | | | UAGGGG) | |
| SEQ | LRRK2_bnPCR_ | CCCTGGTGTGCCCTCTGATGTTT | 1/1 U/G wobble | ADAR1 on target: |
| ID | ID24310_count1321_ | TTATCCCCATTCTAGGGCAGTAG | base pair at −7 | 0.44% |
| NO: | NoLoops_ | GGTGCACTGCCGTGGTGGGCAA | position; | ADAR1 specificity: |
| 28 | gID_11060 | TCTTTGCAATGATGGCAGCATTG | 1/1 G/G mismatch | 0.65 |
| | | GGATACAGTGTGAAGAGCAGCA | at −6 position; | ADAR2 on target: |
| | | | 1/1 U/G wobble | 0.7% |
| | | | base pair at −3 | ADAR2 specificity: |
| | | | position; | 0.39 |
| | | | 1/1 A/C mismatch | ADAR1/2 on target: |
| | | | at 0 position; | 0.68% |
| | | | 1/1 U/C mismatch | ADAR1/2 specificity: |
| | | | at +4 position; | 0.46 |
| | | | 1/1 U/U mismatch | |
| | | | at +8 position; | |
| | | | 2/2 symmetric | |
| | | | bulge at +10 | |
| | | | position (AG- | |
| | | | GG); | |
| | | | 1/1 U/G wobble | |
| | | | base pair at +18 | |
| | | | position; | |
| | | | 1/1 G/G mismatch | |
| | | | at +19 position | |
| SEQ | LRRK2_bnPCR_ | CCCTGGTGTGCCCTCTGATGTTT | 6/6 symmetric | ADAR1 on target: |
| ID | ID24310_count1321_ | TTTAGGGGATTCTAGGGCAGTA | internal loop at | 0.15% |
| NO: | −14_26_gID_02264 | GGGTGCACTGCCGTGGTGGGCA | −14 position | ADAR1 specificity: |
| 29 | | ATCAAACGTATGATGGCAGCAT | (UGCAAA- | 0.82 |
| | | TGGGATACAGTGTGAAGAGCAG | AAACGU); | ADAR2 on target: |
| | | CA | 1/1 U/G wobble | 0.67% |
| | | | base pair at −7 | ADAR2 specificity: |
| | | | position; | 0.72 |
| | | | 1/1 G/G mismatch | ADAR1/2 on target: |
| | | | at −6 position; | 0.64% |
| | | | 1/1 U/G wobble | ADAR1/2 specificity: |
| | | | base pair at −3 | 0.75 |
| | | | position; | |
| | | | 1/1 A/C mismatch | |
| | | | at 0 position; | |
| | | | 1/1 U/C mismatch | |
| | | | at +4 position; | |
| | | | 1/1 U/U mismatch | |
| | | | at +8 position; | |
| | | | 2/2 symmetric | |
| | | | bulge at +10 | |
| | | | position (AG- | |
| | | | GG); | |
| | | | 1/1 U/G wobble at | |
| | | | +18 position; | |
| | | | 1/1 G/G mismatch | |
| | | | at +19 position; | |
| | | | 6/6 symmetric | |
| | | | internal loop at | |
| | | | +26 position | |
| | | | (GGGGAU- | |
| | | | UAGGGG) | |
| SEQ | LRRK2_bnPCR_ | CCCTGGTGTGCCCTCTGATGTTT | 1/1 U/G wobble | ADAR1 on target: |
| ID | ID23393_count2397_ | TTATCCCCATTCTACCGCTGTGC | base pair at −3 | 0.56% |
| NO: | NoLoops_ | TGGGCAATCCCGTGGTCAGCAA | position; | ADAR1 specificity: |
| 30 | gID_09625 | TCTTTGCAATGATGGCAGCATTG | 1/1 A/C mismatch | 0.59 |
| | | GGATACAGTGTGAAGAGCAGCA | at 0 position; | ADAR2 on target: |
| | | | 1/1 C/C mismatch | 0.69% |
| | | | at +2 position; | ADAR2 specificity: |
| | | | 1/1 U/G wobble | 0.41 |
| | | | base pair at +8 | ADAR1/2 on target: |
| | | | position; | 0.67% |
| | | | 1/1 U/G wobble | ADAR1/2 specificity: |
| | | | base pair at +12 | 0.45 |
| | | | position; | |
| | | | 1/1 U/U mismatch | |
| | | | at +15 position; | |
| | | | 1/1 U/C mismatch | |
| | | | at +18 position | |
| SEQ | LRRK2_bnPCR_ | CCCTGGTGTGCCCTCTGATGTTT | 6/6 symmetric | ADAR1 on target: |
| ID | ID23393_count2397_ | TTTAGGGGATTCTACCGCTGTGC | internal loop at | 0.41% |
| NO: | −20_26_gID_06774 | TGGGCAATCCCGTGGTCAGCAA | −20 position | ADAR1 specificity: |
| 31 | | TCTTTGCATACTACGCAGCATTG | (CAUCAU- | 0.67 |
| | | GGATACAGTGTGAAGAGCAGCA | UACUAC); | ADAR2 on target: |
| | | | 1/1 U/G wobble | 0.64% |
| | | | base pair at −3 | ADAR2 specificity: |
| | | | position; | 0.7 |
| | | | 1/1 A/C mismatch | ADAR1/2 on target: |
| | | | at 0 position; | 0.63% |
| | | | 1/1 C/C mismatch | ADAR1/2 specificity: |
| | | | at +2 position; | 0.73 |
| | | | 1/1 U/G wobble | |
| | | | base pair at +8 | |
| | | | position; | |
| | | | 1/1 U/G wobble | |
| | | | base pair at +12 | |
| | | | position; | |
| | | | 1/1 U/U mismatch | |
| | | | at +15 position; | |
| | | | 1/1 U/C mismatch | |
| | | | at +18 position; | |
| | | | 6/6 symmetric | |
| | | | internal loop at | |
| | | | +26 position | |
| | | | (GGGGAU- | |
| | | | UAGGGG) | |
| SEQ | LRRK2_bnPCR_ | CCCTGGTGTGCCCTCTGATGTTT | 6/6 symmetric | ADAR1 on target: |
| ID | ID23393_count2397_ | TTTAGGGGATTCTACCGCTGTGC | internal loop at | 0.39% |
| NO: | −14_26_gID_06456 | TGGGCAATCCCGTGGTCAGCAA | −14 position | ADAR1 specificity: |
| 32 | | TCAAACGTATGATGGCAGCATT | (UGCAAA- | 0.83 |
| | | GGGATACAGTGTGAAGAGCAGC | AAACGU); | ADAR2 on target: |
| | | A | 1/1 U/G wobble | 0.67% |
| | | | base pair at −3 | ADAR2 specificity: |
| | | | position; | 0.75 |
| | | | 1/1 A/C mismatch | ADAR1/2 on target: |
| | | | at 0 position; | 0.62% |
| | | | 1/1 C/C mismatch | ADAR1/2 specificity: |
| | | | at +2 position; | 0.75 |
| | | | 1/1 U/G wobble | |
| | | | base pair at +8 | |
| | | | position; | |
| | | | 1/1 U/G wobble | |
| | | | base pair at +12 | |
| | | | position; | |
| | | | 1/1 U/G mismatch | |
| | | | at +15 position; | |
| | | | 1/1 U/C mismatch | |
| | | | at +18 position; | |
| | | | 6/6 symmetric | |
| | | | internal loop at | |
| | | | +26 position | |
| | | | (GGGGAU- | |
| | | | UAGGGG) | |
| SEQ | LRRK2_bnPCR_ | CCCTGGTGTGCCCTCTGATGTTT | 1/1 U/G wobble at | ADAR1 on target: |
| ID | ID22759_count1590_ | TTATCCCCATTCTACAACAGTAC | −3 position; | 0.48% |
| NO: | NoLoops_ | GGTGAAGTGCCGTGGTCAGCAA | 1/1 A/C mismatch | ADAR1 specificity: |
| 33 | gID_06335 | TCTTTGCAATGATGGCAGCATTG | at 0 position; | 0.5 |
| | | GGATACAGTGTGAAGAGCAGCA | 1/1 U/G wobble | ADAR2 on target: |
| | | | base pair at +4 | 0.68% |
| | | | position; | ADAR2 specificity: |
| | | | 5/5 symmetric | 0.43 |
| | | | internal loop at +6 | ADAR1/2 on target: |
| | | | position | 0.62% |
| | | | (GCUCA- | ADAR1/2 specificity: |
| | | | GGUGA); | 0.46 |
| | | | 1/1 C/A mismatch | |
| | | | at +17 position | |
| SEQ | LRRK2_bnPCR_ | CCCTGGTGTGCCCTCTGATGTTT | 6/6 symmetric | ADAR1 on target: |
| ID | ID22759_count1590_ | TTTAGGGGATTCTACAACAGTA | internal loop at | 0.28% |
| NO: | −14_26_gID_08476 | CGGTGAAGTGCCGTGGTCAGCA | −14 position | ADAR1 specificity: |
| 34 | | ATCAAACGTATGATGGCAGCAT | (UGCAAA- | 0.77 |
| | | TGGGATACAGTGTGAAGAGCAG | AAACGU); | ADAR2 on target: |
| | | CA | 1/1 U/G wobble | 0.68% |
| | | | base pair at −3 | ADAR2 specificity: |
| | | | position; | 0.74 |
| | | | 1/1 A/C mismatch | ADAR1/2 on target: |
| | | | at 0 position; | 0.66% |
| | | | 1/1 U/G wobble | ADAR1/2 specificity: |
| | | | base pair at +4 | 0.77 |
| | | | position; | |
| | | | 5/5 symmetric | |
| | | | internal loop at +6 | |
| | | | position | |
| | | | (GCUCA- | |
| | | | GGUGA); | |
| | | | 1/1 C/A mismatch | |
| | | | at +17 position; | |
| | | | 6/6 symmetric | |
| | | | internal loop at | |
| | | | +26 position | |
| | | | (GGGGAU- | |
| | | | UAGGGG) | |
| SEQ | LRRK2_bnPCR_ | CCCTGGTGTGCCCTCTGATGTTT | 9/9 symmetric | ADAR1 on target: |
| ID | ID22357_count844_ | TTCTAGATATTCTACGGCAGTTC | internal loop at | 0.43% |
| NO: | −22_26_gID_02673 | ATAGCAATCCCGTAGTCAACAA | −19 position | ADAR1 specificity: |
| 35 | | TCTTTGCCATTATTTGAGCATTG | (GCCAUCAUU- | 0.74 |
| | | GGATACAGTGTGAAGAGCAGCA | CAUUAUUUG); | ADAR2 on target: |
| | | | 1/1 C/A mismatch | 0.64% |
| | | | at −8 position; | ADAR2 specificity: |
| | | | 1/1 A/C mismatch | 0.5 |
| | | | at 0 position; | ADAR1/2 on target: |
| | | | 1/1 C/C mismatch | 0.52% |
| | | | at +2 position; | ADAR1/2 specificity: |
| | | | 2/2 symmetric | 0.57 |
| | | | bulge at +9 | |
| | | | position (CA- | |
| | | | AU); | |
| | | | 1/1 U/G mismatch | |
| | | | at +12 position; | |
| | | | 1/1 U/G wobble | |
| | | | base pair at +18 | |
| | | | position; | |
| | | | 1/1 G/U wobble | |
| | | | base pair at +26 | |
| | | | position; | |
| | | | 5/5 symmetric | |
| | | | internal loop at | |
| | | | +27 position | |
| | | | (GGGAU- | |
| | | | CUAGA) | |
| SEQ | LRRK2_bnPCR_ | CCCTGGTGTGCCCTCTGATGTTT | 8/8 symmetric | ADAR1 on target: |
| ID | ID22357_count844_ | TTCGGAGGATTCTACGGCAGTTC | internal loop at | 0.4% |
| NO: | −12_26_gID_07465 | ATAGCAATCCCGTAGTCAACAA | −12 position | ADAR1 specificity: |
| 36 | | GGGTCCCCATGATGGCAGCATT | (UGCAAAGA- | 0.82 |
| | | GGGATACAGTGTGAAGAGCAGC | GGGUCCCC); | ADAR2 on target: |
| | | A | 1/1 C/A mismatch | 0.52% |
| | | | at −8 position; | ADAR2 specificity: |
| | | | 1/1 A/C mismatch | 0.71 |
| | | | at 0 position; | ADAR1/2 on target: |
| | | | 1/1 C/C mismatch | 0.48% |
| | | | at +2 position; | ADAR1/2 specificity: |
| | | | 2/2 symmetric | 0.74 |
| | | | bulge at +9 | |
| | | | position (CA- | |
| | | | AU); | |
| | | | 1/1 U/U mismatch | |
| | | | at +12 position; | |
| | | | 1/1 U/G wobble | |
| | | | base pair at +18 | |
| | | | position; | |
| | | | 6/6 symmetric | |
| | | | internal loop at | |
| | | | +26 position | |
| | | | (GGGGAU- | |
| | | | CGGAGG) | |
| SEQ | LRRK2_bnPCR_ | CCCTGGTGTGCCCTCTGATGTTT | 8/8 symmetric | ADAR1 on target: |
| ID | ID22357_count844_ | TTATCCCCATCACCCTGCAGTTC | internal loop at | 0.3% |
| NO: | −12_18_gID_11904 | ATAGCAATCCCGTAGTCAACAA | −12 position | ADAR1 specificity: |
| 37 | | CAATATCCATGATGGCAGCATT | (UGCAAAGA- | 0.79 |
| | | GGGATACAGTGTGAAGAGCAGC | CAAUAUCC); | ADAR2 on target: |
| | | A | 1/1 C/A mismatch | 0.68% |
| | | | at −8 position; | ADAR2 specificity: |
| | | | 1/1 A/C mismatch | 0.67 |
| | | | at 0 position; | ADAR1/2 on target: |
| | | | 1/1 C/C mismatch | 0.65% |
| | | | at +2 position; | ADAR1/2 specificity: |
| | | | 2/2 symmetric | 0.73 |
| | | | bulge at +9 | |
| | | | position (CA- | |
| | | | AU); | |
| | | | 1/1 U/U mismatch | |
| | | | at +12 position; | |
| | | | 6/6 symmetric | |
| | | | internal loop at | |
| | | | +18 position | |
| | | | (UGUAGA- | |
| | | | CACCCU) | |
| SEQ | LRRK2_bnPCR_ | CCCTGGTGTGCCCTCTGATGTTT | 6/6 symmetric | ADAR1 on target: |
| ID | ID16690_count1976_ | TTCTGGTGATTCTACAACAGTAC | internal loop at | 0.5% |
| NO: | −22_26_gID_01893 | TGAGCTATCCCGAATTCAACAA | −22 position | ADAR1 specificity: |
| 38 | | TCTTTGCAATAAGCGAAGCATT | (GCCAUC- | 0.66 |
| | | GGGATACAGTGTGAAGAGCAGC | AAGCGA); | ADAR2 on target: |
| | | A | 1/1 C/A mismatch | 0.64% |
| | | | at −8 position; | ADAR2 specificity: |
| | | | 10/10 symmetric | 0.51 |
| | | | internal loop at −4- | ADAR1/2 on target: |
| | | | >5 position | 0.6% |
| | | | (CUACAGCAUU- | ADAR1/2 specificity: |
| | | | UAUCCCGAAU); | 0.56 |
| | | | 1/1 C/A mismatch | |
| | | | at +17 position; | |
| | | | 1/1 G/G mismatch | |
| | | | at +26 position; | |
| | | | 1/1 G/U wobble | |
| | | | base pair at +27 | |
| | | | position; | |
| | | | 4/4 symmetric | |
| | | | bulge at +28 | |
| | | | position (GGAU- | |
| | | | CUGG) | |
| SEQ | LRRK2_bnPCR_ | CCCTGGTGTGCCCTCTGATGTTT | 6/6 symmetric | ADAR1 on target: |
| ID | ID16690_count1976_ | TTATCCCCATTCGAAAAAAGTA | internal loop at | 0.22% |
| NO: | −22_16_gID_06801 | CTGAGCTATCCCGAATTCAACA | −22 position | ADAR1 specificity: |
| 39 | | ATCTTTGCAATCAATCAAGCATT | (GCCAUC- | 0.7 |
| | | GGGATACAGTGTGAAGAGCAGC | CAAUCA); | ADAR2 on target: |
| | | A | 1/1 C/A mismatch | 0.61% |
| | | | at −8 position; | ADAR2 specificity: |
| | | | 10/10 symmetrica | 0.61 |
| | | | internal loop at −4- | ADAR1/2 on target: |
| | | | >5 position | 0.57% |
| | | | (CUACAGCAUU- | ADAR1/2 specificity: |
| | | | UAUCCCGAAU); | 0.61 |
| | | | 6/6 symmetric | |
| | | | internal loop at | |
| | | | +16 position | |
| | | | (GCUGUA- | |
| | | | GAAAAA) | |
| SEQ | LRRK2_bnPCR_ | CCCTGGTGTGCCCTCTGATGTTT | 6/6 symmetric | ADAR1 on target: |
| ID | ID16690_count1976_ | TTATCCCCATTCGAATAAAGTAC | internal loop at | 0.22% |
| NO: | −16_16_gID_10719 | TGAGCTATCCCGAATTCAACAA | −16 position | ADAR1 specificity: |
| 40 | | TCTTCTATTAGATGGCAGCATTG | (AUUGCA- | 0.8 |
| | | GGATACAGTGTGAAGAGCAGCA | CUAUUA); | ADAR2 on target: |
| | | | 1/1 C/A mismatch | 0.66% |
| | | | at −8 position; | ADAR2 specificity: |
| | | | 10/10 symmetric | 0.67 |
| | | | internal loop at −4- | ADAR1/2 on target: |
| | | | >5 position | 0.6% |
| | | | (CUACAGCAUU- | ADAR1/2 specificity: |
| | | | UAUCCCGAAU); | 0.7 |
| | | | 6/6 symmetric | |
| | | | internal loop at | |
| | | | +16 position | |
| | | | (GCUGUA- | |
| | | | GAAUAA) | |
| SEQ | LRRK2_bnPCR_ | CCCTGGTGTGCCCTCTGATGTTT | 1/1 C/U mismatch | ADAR1 on target: |
| ID | ID14524_count2063_ | TTATCCCCATTCTACAGCAGTAG | at −8 position; | 0.49% |
| NO: | NoLoops_ | TGTGCAGTGCCGTGGTCATCAAT | 1/1 U/G wobble | ADAR1 specificity: |
| 41 | gID_01159 | CTTTGCAATGATGGCAGCATTG | base pair at −3 | 0.54 |
| | | GGATACAGTGTGAAGAGCAGCA | position; | ADAR2 on target: |
| | | | 1/1 A/C mismatch | 0.7% |
| | | | at 0 position; | ADAR2 specificity: |
| | | | 1/1 U/g wobble | 0.38 |
| | | | base pair at +4 | ADAR1/2 on target: |
| | | | position; | 0.66% |
| | | | 1/1 U/U mismatch | ADAR1/2 specificity: |
| | | | at +8 position; | 0.43 |
| | | | 1/1 G/G mismatch | |
| | | | at +11 position | |
| SEQ | LRRK2_bnPCR_ | CCCTGGTGTGCCCTCTGATGTTT | 6/6 symmetric | ADAR1 on target: |
| ID | ID14524_count2063_ | TTTAGGGGATTCTACAGCAGTA | internal loop at | 0.31% |
| NO: | −14_26_gID_05349 | GTGTGCAGTGCCGTGGTCATCA | −14 position | ADAR1 specificity: |
| 42 | | ATCAAACGTATGATGGCAGCAT | (UGCAAA- | 0.77 |
| | | TGGGATACAGTGTGAAGAGCAG | AAACGU); | ADAR2 on target: |
| | | CA | 1/1 C/U mismatch | 0.72% |
| | | | at −8 position; | ADAR2 specificity: |
| | | | 1/1 U/G wobble | 0.69 |
| | | | base pair at −3 | ADAR1/2 on target: |
| | | | position; | 0.68% |
| | | | 1/1 A/C mismatch | ADAR1/2 specificity: |
| | | | at 0 position; | 0.73 |
| | | | 1/1 U/G wobble | |
| | | | base pair at +4 | |
| | | | position; | |
| | | | 1/1 U/U mismatch | |
| | | | at +8 position; | |
| | | | 1/1 G/G mismatch | |
| | | | at +11 position; | |
| | | | 6/6 symmetric | |
| | | | internal loop at | |
| | | | +26 position | |
| | | | (GGGGAU- | |
| | | | UAGGGG) | |
| SEQ | LRRK2_bnPCR_ | CCCTGGTGTGCCCTCTGATGTTT | 6/6 symmetric | ADAR1 on target: |
| ID | ID8030_count919_ | TTCAGTAGCTTCTACAGCAGTTC | internal loop at | 0.37% |
| NO: | −22_26_gID_12156 | GGAGGAATCCCGAGGTCAGCAA | −22 position | ADAR1 specificity: |
| 43 | | TCTTTGCAATAAGAGAAGCATT | (GCCAUC- | 0.68 |
| | | GGGATACAGTGTGAAGAGCAGC | AAGAGA); | ADAR2 on target: |
| | | A | 1/1 U/G wobble | 0.6% |
| | | | base pair at −3 | ADAR2 specificity: |
| | | | position; | 0.61 |
| | | | 1/1 A/A mismatch | ADAR1/2 on target: |
| | | | at −2 position; | 0.56% |
| | | | 1/1 A/C mismatch | ADAR1/2 specificity: |
| | | | at 0 position; | 0.68 |
| | | | 1/1 C/C mismatch | |
| | | | at +2 position; | |
| | | | 1/1 G/G mismatch | |
| | | | at +6 position; | |
| | | | 3/3 symmetric | |
| | | | bulge at +10 | |
| | | | position (AGU- | |
| | | | UCG); | |
| | | | 7/7 symmetric | |
| | | | internal loop at | |
| | | | +25 position | |
| | | | (UGGGGAU- | |
| | | | CAGUAGC) | |
| SEQ | LRRK2_bnPCR_ | CCCTGGTGTGCCCTCTGATGTTT | 6/6 symmetric | ADAR1 on target: |
| ID | ID8030_count919_ | TTTAGGGGCTTCTACAGCAGTTC | internal loop at | 0.37% |
| NO: | −20_26_gID_00460 | GGAGGAATCCCGAGGTCAGCAA | −20 position | ADAR1 specificity: |
| 44 | | TCTTTGCATACTACGCAGCATTG | (CAUCAU- | 0.72 |
| | | GGATACAGTGTGAAGAGCAGCA | UACUAC); | ADAR2 on target: |
| | | | 1/1 U/G wobble | 0.59% |
| | | | base pair at −3 | ADAR2 specificity: |
| | | | position; | 0.67 |
| | | | 1/1 A/A mismatch | ADAR1/2 on target: |
| | | | at −2 position; | 0.53% |
| | | | 1/1 A/C mismatch | ADAR1/2 specificity: |
| | | | at 0 position; | 0.72 |
| | | | 1/1 C/C mismatch | |
| | | | at +2 position; 1/1 | |
| | | | G/G mismatch at | |
| | | | +6 position; | |
| | | | 3/3 symmetric | |
| | | | bulge at +10 | |
| | | | position (AGU- | |
| | | | UCG); | |
| | | | 7/7 symmetric | |
| | | | internal loop at | |
| | | | +25 position | |
| | | | (UGGGGAU- | |
| | | | UAGGGGC) | |
| SEQ | LRRK2_bnPCR_ | CCCTGGTGTGCCCTCTGATGTTT | 6/6 symmetric | ADAR1 on target: |
| ID | ID8030_count919_ | TTATGGAACGTCTACAGCAGTTC | internal loop at | 0.36% |
| NO: | −18_24_gID_04981 | GGAGGAATCCCGAGGTCAGCAA | −18 position | ADAR1 specificity: |
| 45 | | TCTTTGGTAGCTTGGCAGCATTG | (UCAUUG- | 0.77 |
| | | GGATACAGTGTGAAGAGCAGCA | GUAGCU); | ADAR2 on target: |
| | | | 1/1 U/G wobble | 0.63% |
| | | | base pair at −3 | ADAR2 specificity: |
| | | | position; | 0.67 |
| | | | 1/1 A/A mismatch | ADAR1/2 on target: |
| | | | at −2 position; | 0.57% |
| | | | 1/1 A/C mismatch | ADAR1/2 specificity: |
| | | | at 0 position; | 0.7 |
| | | | 1/1 C/C mismatch | |
| | | | at +2 position; | |
| | | | 1/1 G/G mismatch | |
| | | | at +6 position; | |
| | | | 3/3 symmetric | |
| | | | bulge at +10 | |
| | | | position (AGU- | |
| | | | UCG); | |
| | | | 6/6 symmetric | |
| | | | internal loop at | |
| | | | +24 position | |
| | | | (AUGGGG- | |
| | | | GGAACG) | |
| SEQ | LRRK2_bnPCR_ | CCCTGGTGTGCCCTCTGATGTTT | 6/6 symmetric | ADAR1 on target: |
| ID | ID8030_count919_ | TTATCCAGCGGGTACAGCAGTT | internal loop at | 0.33% |
| NO: | −18 22 gID_06761 | CGGAGGAATCCCGAGGTCAGCA | −18 position | ADAR1 specificity: |
| 46 | | ATCTTTGGTCTTCTGGCAGCATT | (UCAUUG- | 0.78 |
| | | GGGATACAGTGTGAAGAGCAGC | GUCUUC); | ADAR2 on target: |
| | | A | 1/1 U/G wobble | 0.62% |
| | | | base pair at −3 | ADAR2 specificity: |
| | | | position; | 0.69 |
| | | | 1/1 A/A mismatch | ADAR1/2 on target: |
| | | | at −2 position; | 0.5% |
| | | | 1/1 A/C mismatch | ADAR1/2 specificity: |
| | | | at 0 position; | 0.73 |
| | | | 1/1 C/C mismatch | |
| | | | at +2 position; | |
| | | | 1/1 G/G mismatch | |
| | | | at +6 position; | |
| | | | 3/3 symmetric | |
| | | | bulge at +10 | |
| | | | position (AGU- | |
| | | | UCG); | |
| | | | 6/6 symmetric | |
| | | | internal loop at | |
| | | | +22 position | |
| | | | (GAAUGG- | |
| | | | AGCGGG) | |
| SEQ | LRRK2_bnPCR_ | CCCTGGTGTGCCCTCTGATGTTT | 6/6 symmetric | ADAR1 on target: |
| ID | ID8030_count919_ | CAACGGCCCTTCTACAGCAGTTC | internal loop at | 0.43% |
| NO: | −16_28_gID_07038 | GGAGGAATCCCGAGGTCAGCAA | −16 position | ADAR1 specificity: |
| 47 | | TCTTACCCTGGATGGCAGCATTG | (AUUGCA- | 0.75 |
| | | GGATACAGTGTGAAGAGCAGCA | ACCCUG); | ADAR2 on target: |
| | | | 1/1 U/G wobble | 0.56% |
| | | | base pair at −3 | ADAR2 specificity: |
| | | | position; | 0.65 |
| | | | 1/1 A/A mismatch | ADAR1/2 on target: |
| | | | at −2 position; | 0.53% |
| | | | 1/1 A/C mismatch | ADAR1/2 specificity: |
| | | | at 0 position; | 0.66 |
| | | | 1/1 C/C mismatch | |
| | | | at +2 position; | |
| | | | 1/1 G/G mismatch | |
| | | | at +6 position; | |
| | | | 3/3 symmetric | |
| | | | bulge at +10 | |
| | | | position (AGU- | |
| | | | UCG); | |
| | | | 1/0 asymmetric | |
| | | | bulge at +25 | |
| | | | position (U-); | |
| | | | 5/6 asymmetric | |
| | | | internal loop at | |
| | | | +29 position | |
| | | | (GAUAA- | |
| | | | CAACGG) | |
| SEQ | LRRK2_bnPCR_ | CCCTGGTGTGCCCTCTGATGTTT | 6/6 symmetric | ADAR1 on target: |
| ID | ID8030_count919_ | TTATTGAGCCTCTACAGCAGTTC | internal loop at | 0.38% |
| NO: | −16_24_gID_09086 | GGAGGAATCCCGAGGTCAGCAA | −16 position | ADAR1 specificity: |
| 48 | | TCTTAAATTAGATGGCAGCATTG | (AUUGCA- | 0.81 |
| | | GGATACAGTGTGAAGAGCAGCA | AAAUUA); | ADAR2 on target: |
| | | | 1/1 U/G wobble | 0.58% |
| | | | base pair at −3 | ADAR2 specificity: |
| | | | position; | 0.69 |
| | | | 1/1 A/A mismatch | ADAR1/2 on target: |
| | | | at −2 position; | 0.52% |
| | | | 1/1 A/C mismatch | ADAR1/2 specificity: |
| | | | at 0 position; | 0.72 |
| | | | 1/1 C/C mismatch | |
| | | | at +2 position; | |
| | | | 1/1 G/G mismatch | |
| | | | at +6 position; | |
| | | | 3/3 symmetric | |
| | | | bulge at +10 | |
| | | | position (AGU- | |
| | | | UCG); | |
| | | | 5/5 symmetric | |
| | | | internal loop at | |
| | | | +24 position | |
| | | | (AUGGG- | |
| | | | GAGCC); | |
| | | | 1/1 G/U wobble | |
| | | | base pair at +29 | |
| | | | position | |
| SEQ | LRRK2_bnPCR_ | CCCTGGTGTGCCCTCTGATGTTT | 6/6 symmetric | ADAR1 on target: |
| ID | ID8030_count919_ | TTTAGGGGCTTCTACAGCAGTTC | internal loop at | 0.35% |
| NO: | −14_26_gID_07899 | GGAGGAATCCCGAGGTCAGCAA | −14 position | ADAR1 specificity: |
| 49 | | TCAAACGTATGATGGCAGCATT | (UGCAAA- | 0.82 |
| | | GGGATACAGTGTGAAGAGCAGC | AAACGU); | ADAR2 on target: |
| | | A | 1/1 U/G wobble | 0.59% |
| | | | base pair at −3 | ADAR2 specificity: |
| | | | position; | 0.73 |
| | | | 1/1 A/A mismatch | ADAR1/2 on target: |
| | | | at −2 position; | 0.55% |
| | | | 1/1 A/C mismatch | ADAR1/2 specificity: |
| | | | at 0 position; | 0.75 |
| | | | 1/1 C/C mismatch | |
| | | | at +2 position; | |
| | | | 1/1 G/G mismatch | |
| | | | at +6 position; | |
| | | | 3/3 symmetric | |
| | | | bulge at +10 | |
| | | | position (AGU- | |
| | | | UCG); | |
| | | | 7/7 symmetric | |
| | | | internal loop at | |
| | | | +25 position | |
| | | | (UGGGGAU- | |
| | | | UAGGGGC) | |
| SEQ | LRRK2_bnPCR_ | CCCTGGTGTGCCCTCTGATGTTT | 6/6 symmetric | ADAR1 on target: |
| ID | ID8030_count919_ | TTATGAGGCGTCTACAGCAGTTC | internal loop at | 0.46% |
| NO: | −12 24 gID_03363 | GGAGGAATCCCGAGGTCAGCAA | −12 position | ADAR1 specificity: |
| 50 | | GACTAACAATGATGGCAGCATT | (CAAAGA- | 0.83 |
| | | GGGATACAGTGTGAAGAGCAGC | GACUAA); | ADAR2 on target: |
| | | A | 1/1 U/G wobble | 0.67% |
| | | | base pair at −3 | ADAR2 specificity: |
| | | | position; | 0.7 |
| | | | 1/1 A/A mismatch | ADAR1/2 on target: |
| | | | at −2 position; | 0.58% |
| | | | 1/1 A/C mismatch | ADAR1/2 specificity: |
| | | | at 0 position; | 0.76 |
| | | | 1/1 C/C mismatch | |
| | | | at +2 position; | |
| | | | 1/1 G/G mismatch | |
| | | | at +6 position; | |
| | | | 3/3 symmetric | |
| | | | bulge at +10 | |
| | | | position (AGU- | |
| | | | UCG); | |
| | | | 6/6 symmetric | |
| | | | internal loop at | |
| | | | +24 position | |
| | | | (AUGGGG- | |
| | | | GAGGCG) | |
| SEQ | LRRK2_bnPCR_ | CCCTGGTGTGCCCTCTGATGTTT | 6/6 symmetric | ADAR1 on target: |
| ID | ID8030_count919_ | TTATTGAGCCTCTACAGCAGTTC | internal loop at | 0.5% |
| NO: | −10_24_gID_09672 | GGAGGAATCCCGAGGTCAGCTT | −10 position | ADAR1 specificity: |
| 51 | | AGCCTGCAATGATGGCAGCATT | (AAGAUU- | 0.84 |
| | | GGGATACAGTGTGAAGAGCAGC | UUAGCC); | ADAR2 on target: |
| | | A | 1/1 U/G wobble | 0.48% |
| | | | base pair at −3 | ADAR2 specificity: |
| | | | position; | 0.75 |
| | | | 1/1 A/A mismatch | ADAR1/2 on target: |
| | | | at −2 position; | 0.59% |
| | | | 1/1 A/C mismatch | ADAR1/2 specificity: |
| | | | at 0 position; | 0.77 |
| | | | 1/1 C/C mismatch | |
| | | | at +2 position; | |
| | | | 1/1 G/G mismatch | |
| | | | at +6 position; | |
| | | | 3/3 symmetric | |
| | | | bulge (AGU- | |
| | | | UCG); | |
| | | | 5/5 symmetric | |
| | | | internal loop at | |
| | | | +24 position | |
| | | | (AUGGG- | |
| | | | GAGCC); | |
| | | | 1/1 G/U wobble | |
| | | | base pair at +29 | |
| | | | position | |

1AA

Machine Learning to Predict Percent Target Editing and Specificity Score of an Engineered Guide that target LRRK2 mRNA.

This example describes using machine learning to predict on-target editing (percentage of editing of the target adenosine in the LRRK2 mRNA) and a specificity score ((on-target edits of the target adenosine in the LRRK2 miRNA)/(sum of off-target edits in the LRRK2 mRNA)) based on an engineered guide RNA sequence. A set of 70,743 guides targeting LRRK2 mRNA, in which the guide RNAs of this set form various structural features in the guide-target RNA scaffold, was used to train and test a convolutional neural network (CNN). Of this set of guides, 60% were used to train the model and 40% were used to test the accuracy of the CNN for predicting on-target editing and specificity score based on an engineered guide sequence. There was a high correlation between the predicted on-target editing and specificity score and the experimentally tested on-target editing and specificity score, indicating that the trained CNN accurately predicts on-target editing and specificity score based on an engineered guide sequence. The experimental testing was done in a cell-free system via high throughput screening of self-annealing guide RNAs linked to target RNAs by a hairpin and using ADAR1 and/or ADAR2 to perform the editing.

### Example 3

Machine Learning for Engineered Guides that Target LRRK2 mRNA

This example describes generating engineered guide RNA sequences that target LRRK2 mRNA based on a specified on-target editing and a specified specificity score using machine learning. The trained CNN of EXAMPLE 2 was used in reverse, in which a specified on-target editing and specified specificity score was inputted into the trained CNN to predict an engineered guide RNA sequence having that target editing and specificity score. 768 engineered guide sequences were generated. The generated guide RNAs on-target editing and specificity score were then experimentally tested as described in EXAMPLE 2 by high-throughput screen. There was a high correlation between the inputted specified on-target editing and specificity score and the experimentally measured on-target editing and specificity score, with a Spearman correlation coefficient of 0.74 for on-target editing and 0.67 for the specificity score. This indicates the trained CNN accurately generated engineered guide sequences based on the on-target editing and specificity score inputs. Exemplary engineered guide RNA sequences generated by the trained CNN and having a high on-target editing and/or high specificity score are SEQ ID NO: 52 to SEQ ID NO: 101.

### Example 4

Machine Learning for Determining gRNA Features that Impact LRRK2 mRNA Editing

This example describes using machine learning to determine features of a guide RNA that impact on-target editing and specificity score for editing a LRRK2 mRNA. A set of 1709 engineered guide RNAs was used to train and test a random forest (RF) model. Of this set of guides, 1000 engineered guides were used to train the RF model and 709 engineered guides were used to test the accuracy of the trained RF model for predicting on-target editing and specificity score based on an engineered guide sequence. There was a high correlation between the predicted on-target editing and specificity score and the experimentally tested on-target editing and specificity score, indicating that the trained RF model accurately predicts on-target editing and specificity score based on an engineered guide sequence. This trained RF model was then used to determine features of the guide RNAs that impact on-target editing and specificity score, such as length of time for editing (20 sec, 1 min, 3 min, 10 min, 30 min, or 60 min), the ADAR used for editing (ADAR1, ADAR2, or ADAR1 and ADAR2), positioning of a right barbell (relative to the target adenosine to be edited), positioning of left barbell (relative to the target nucleotide to be edited). The right barbell positioning was the most important feature for predicting specificity of an engineered guide RNA and the third most important feature for predicting on-target editing. For engineered guide RNAs using ADAR1 for editing, the best positioning of the right barbell in an engineered guide RNA to achieve a high specificity score was +28 or +30 nts, wherein the positioning is relative to the target adenosine in the LRRK2 mRNA to be edited. For engineered guide RNAs using ADAR2 for editing, the best positioning of the right barbell in an engineered guide RNA to achieve a high specificity score was +24 or +26 nts, wherein the positioning is relative to the target adenosine in the LRRK2 mRNA to be edited.

### Example 5

Machine Learning for an Engineered Guide RNA that Targets LRRK2 mRNA

This example describes using machine learning to determine identities of nucleotides at specific positions in engineered guide RNAs that target LRRK2 mRNA to achieve high on-target editing. Machine learning was performed using a Logistic Regression model trained on a set of engineered guide RNAs. Logistic regression coefficients were extracted from the Logistic Regression model. The trained RF model from EXAMPLE 4 was also used. Shapley values were extracted from this trained RF model. The Shapley values and the logistic regression coefficients were then assessed for overlapping nucleotides at specific positions in the engineered guide RNAs that had high on-target editing. This overlap was used to determine the identities of nucleotides at specific positioning in engineered guide RNAs that target LRRK2 mRNA that achieve high target editing. These nucleotides and positions in the engineered guide RNA are as follows: T at position −7, T at position −6, G at position −3, A at position −2, G at position −1, C at position 1, C at position 2, G at position 4, and T at position 10, wherein these positions are relative to the target adenosine in the LRRK2 mRNA to be edited.

### Example 6

### Engineered Guide RNAs Targeting LRRK2

This example describes the on-target and off-target editing efficiencies of various engineered guide RNAs targeting the LRRK2 G2019S mutation. A side-by-side comparison of engineered guide RNAs with varying structural features in the guide-target RNA scaffold that forms upon hybridization of the engineered guide RNA to a target LRRK2 mRNA was assessed in trans. Engineered guide RNAs were dosed in vitro in cells expressing ADAR1. RNA editing at the on-target adenosine and at local off-target adenosines was assessed by Sanger sequencing.

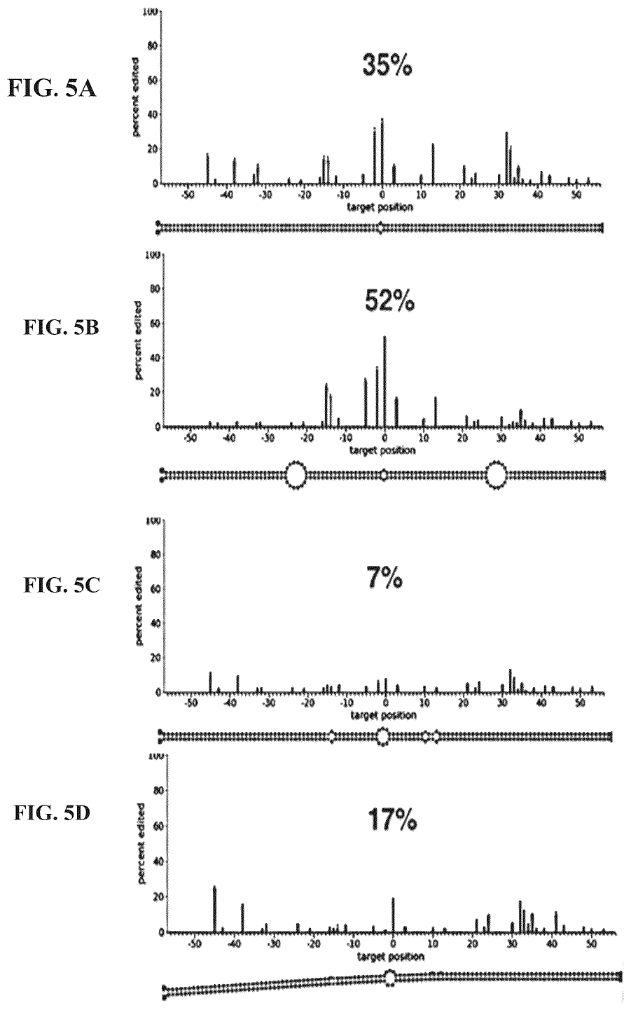

FIGS. 5A-D show the editing profiles of four engineered guide RNAs, including an engineered guide RNA that forms an A/C mismatch structural feature at the target adenosine (FIG. 5A), an engineered guide RNA that forms an A/C mismatch structural feature at the target adenosine and barbells at the −20 and +26 positions relative to the target adenosine (position 0) (FIG. 5B), an engineered guide RNA that forms A/G mismatch structural features at local off-target adenosines at the −14, −2, +10, and +13 positions relative to the target adenosine (position 0) (FIG. 5C), and an engineered guide RNA that forms a I/O asymmetrical bulge at local off-target adenosines at the −14, −2, +10, and +13 positions relative to the target adenosine (position 0) by deletion of a U opposite the local off-target adenosine (FIG. 5D). The plots shown in FIGS. 5A-D depict the position along the target RNA on the x-axis and report percent editing on the y-axis. A diagram of the guide-target RNA scaffold for each engineered guide RNA is shown directly below each graph. As shown in FIGS. 5A-D, simple addition of A/G mismatches or I/O bulges at local off-target adenosines resulted in abrogated on-target editing as compared to the A/C mismatch engineered guide RNA. The addition of a barbell macro-footprint resulted in increase of on-target editing as compared to the A/C mismatch engineered guide RNA.

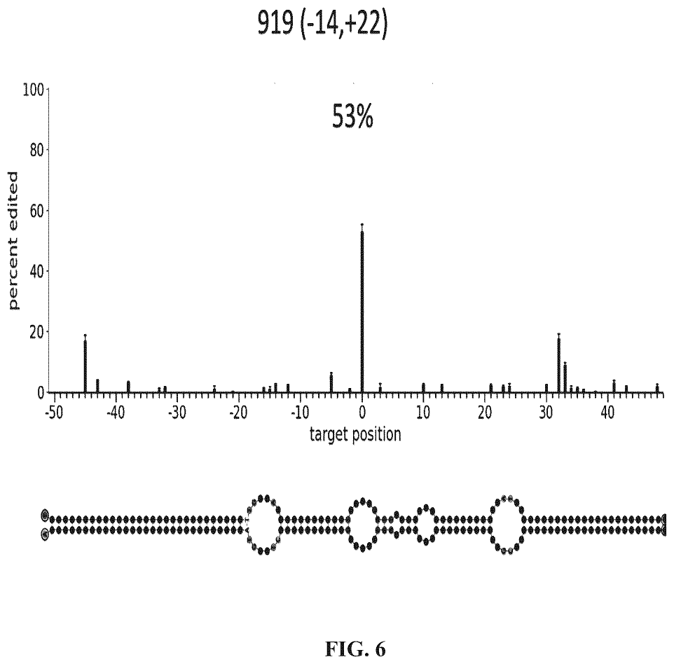

An engineered guide RNA that forms a barbell macro-footprint at the −14 and +22 positions relative to the target adenosine (position 0) and which also forms a micro-footprint comprising other structural features was also evaluated in cells (FIG. 6). This engineered guide RNA displayed a boost in on-target adenosine editing and a reduction in local off-target adenosine editing as compared to the A/C mismatch engineered guide RNA.

### Example 7

### In-Cell Editing Efficiencies for Machine Learning Derived LRRK2 Engineered Guide RNAs (−20,+26)

This example describes targeting the LRRK2 G2019S mutation for editing in vitro, in cells, using engineered guide RNAs of the present disclosure that were derived using multiple machine learning (ML) models, including an exhaustive ML model and a generative ML model. 750 ng of plasmid was transfected in cells (20,000 cells/well) of 68 cell line (LRRK2 cDNA minigene+ADAR1) and 219 cell line (LRRK2 cDNA minigene+ADAR1+2). 4 technical replicates were performed for each engineered guide RNA. Each engineered guide RNA tested contained a barbell macro-footprint of symmetrical internal loops with coordinates at −20 and +26 relative to the target A.

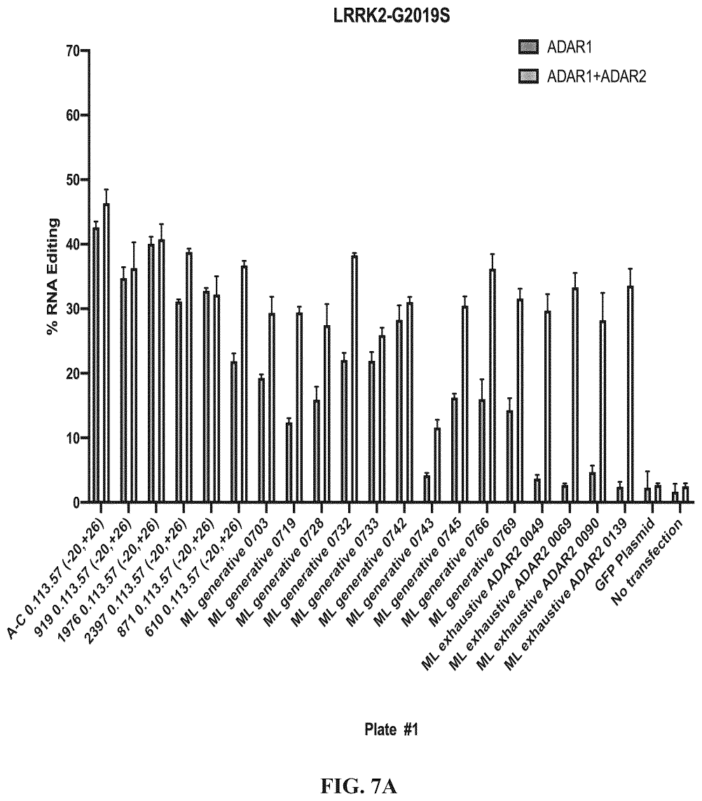

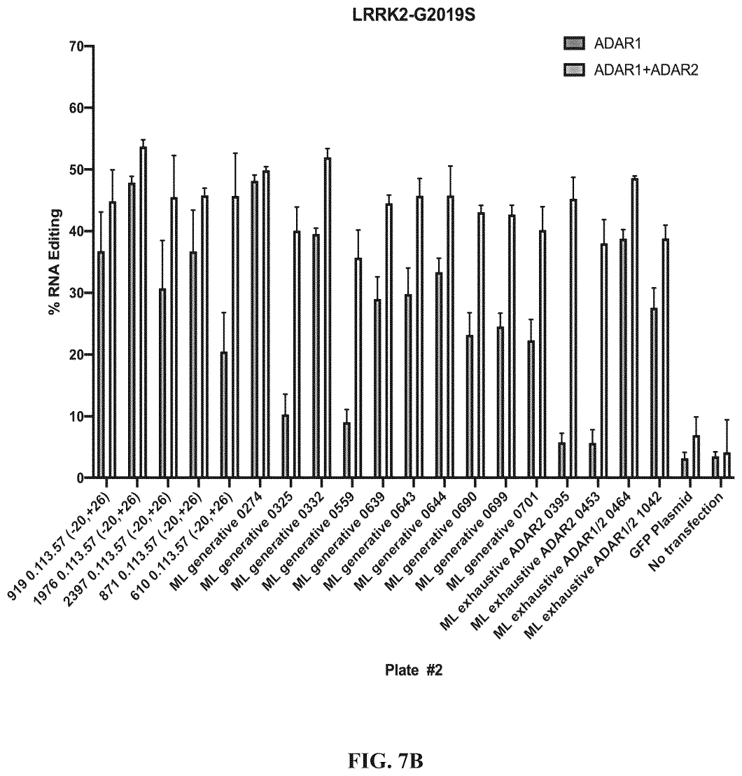

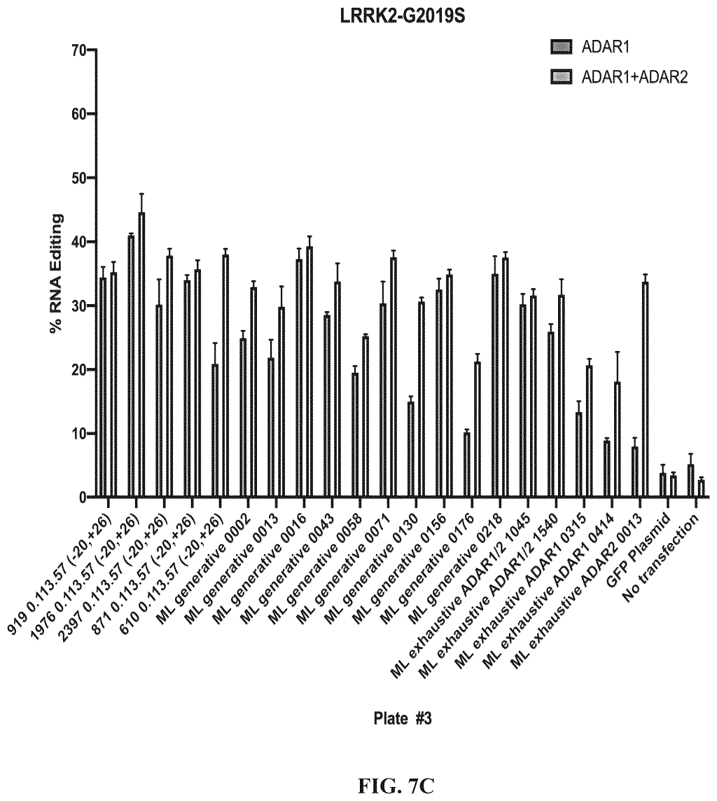

FIG. 7A-FIG. 7C provide a summary of the RNA editing efficiency of each LRRK2 engineered guide RNA tested via ADAR. The following LRRK2 engineered guide RNAs recited in TABLE 3 are utilized in the summary of RNA editing provided in FIG. 7A-FIG. 7C. Each engineered guide RNA sequence can also be represented as a DNA sequence in which each U is replaced with a T.

TABLE 3
LRRK2 ML Engineered Guide RNA Sequences

| SEQ | | Engineered | | Left | Right |
|---|---|---|---|---|---|
| ID | Guide | Guide RNA | | Barbell | Barbell |
| NO | Name | Sequence | Structural Features (target/guide) | Position | Position |
| 106 | Ml_ | CCCUGGUGUG | −49_1-1_mismatch_C-A | −20 | 26 |
| | Ex- | CCCUCUGAUG | −20_6-6_internal_loop-symmetric_CAUCAU-UACUAC | | |
| | haustive_ | UUUUUUAGGG | −13_1-1_mismatch_G-A | | |
| | ADAR1_ | GAUUCUACAG | −3_1-1_wobble_U-G | | |
| | 0315 | CAGGACUGAG | 1_1-1_wobble_G-U | | |
| | | CAAUCUUGUG | 2_1-1_mismatch_C-C | | |
| | | GUCAGCAAUA | 13_1-1_mismatch_A-G | | |
| | | UUUGCAUACU | 26_6-6_internal_loop-symmetric_GGGGAU-UAGGGG | | |
| | | ACGCAGCAUU | 34_1-1_wobble_G-U | | |
| | | GGGAUACAGU | | | |
| | | GUGAAAAGCA | | | |
| | | GCA | | | |
| 107 | Ml_ | CCCUGGUGUG | −49_1-1_mismatch_C-A | −20 | 26 |
| | Ex- | CCCUCUGAUG | −20_6-6_internal_loop-symmetric_CAUCAU-UACUAC | | |
| | haustive_ | UUUUUUAGGG | −13_1-1_mismatch_G-A | | |
| | ADAR1_ | GAUUCUACAG | −3_1-1_wobble_U-G | | |
| | 0414 | CAGGACUGAG | 0->1_2-2_bulge-symmetric_AG-GA | | |
| | | CAAUGGAGUG | 13_1-1_mismatch_A-G | | |
| | | GUCAGCAAUA | 26_6-6_internal_loop-symmetric_GGGGAU-UAGGGG | | |
| | | UUUGCAUACU | 34_1-1_wobble_G-U | | |
| | | ACGCAGCAUU | | | |
| | | GGGAUACAGU | | | |
| | | GUGAAAAGCA | | | |
| | | GCA | | | |
| 108 | ML_ | CCCUGGUGUG | −49_1-1_mismatch_C-A | −20 | 26 |
| | Ex- | CCCUCUGAUG | −20_6-6_internal_loop-symmetric_CAUCAU-UACUAC | | |
| | haustive_ | UUUUUUAGGG | −10_1-1_mismatch_U-C | | |
| | ADAR2_ | GAUUCUACAG | −1->0_2-2_bulge-symmetric_CA-GU | | |
| | 0013 | CACGACUGAG | 4_1-1_wobble_U-G | | |
| | | CAGUGCGUUA | 13_2-2_bulge-symmetric_AC-CG | | |
| | | GUCAGCCAUC | 26_6-6_internal_loop-symmetric_GGGGAU-UAGGGG | | |
| | | UUUGCAUACU | 34_1-1_wobble_G-U | | |
| | | ACGCAGCAUU | | | |
| | | GGGAUACAGU | | | |
| | | GUGAAAAGCA | | | |
| | | GCA | | | |
| 109 | Ml_ | CCCUGGUGUG | −49_1-1_mismatch_C-A | −20 | 26 |
| | Ex- | CCCUCUGAUG | −20_6-6_internal_loop-symmetric_CAUCAU-UACUAC | | |
| | haustive_ | UUUUUUAGGG | −11_1-1_mismatch_U-C | | |
| | ADAR2_ | GAUUCUACAG | −1->0_2-2_bulge-symmetric_CA-GU | | |
| | 0049 | CAUUACUCAG | 4_1-1_wobble_U-G | | |
| | | CAGUGCGUUA | 9_1-1_mismatch_C-C | | |
| | | GUCAGCACUC | 14_1-1_mismatch_C-U | | |
| | | UUUGCAUACU | 26_6-6_internal_loop-symmetric_GGGGAU-UAGGGG | | |
| | | ACGCAGCAUU | 34_1-1_wobble_G-U | | |
| | | GGGAUACAGU | | | |
| | | GUGAAAAGCA | | | |
| | | GCA | | | |
| 110 | Ml_ | CCCUGGUGUG | −49_1-1_mismatch_C-A | −20 | 26 |
| | Ex- | CCCUCUGAUG | −20_6-6_internal_loop-symmetric_CAUCAU-UACUAC | | |
| | haustive_ | UUUUUUAGGG | −7_1-1_wobble_U-G | | |
| | ADAR2_ | GAUUCUACAG | −1->0_2-2_bulge-symmetric_CA-GU | | |
| | 0269 | CACGACUGAG | 4_1-1_wobble_U-G | | |
| | | CAGUGCGUUA | 13_2-2_bulge-symmetric_AC-CG | | |
| | | GUCGGCAAUC | 26_6-6_internal_loop-symmetric_GGGGAU-UAGGGG | | |
| | | UUUGCAUACU | 34_1-1_wobble_G-U | | |
| | | ACGCAGCAUU | | | |
| | | GGGAUACAGU | | | |
| | | GUGAAAAGCA | | | |
| | | GCA | | | |
| 111 | ML_ | CCCUGGUGUG | −49_1-1_mismatch_C-A | −20 | 26 |
| | Ex- | CCCUCUGAUG | −20_6-6_internal_loop-symmetric_CAUCAU-UACUAC | | |
| | haustive_ | UUUUUUAGGG | −2->0_3-3_bulge-symmetric_ACA-GUG | | |
| | ADAR2_ | GAUUCUACAG | 4_1-1_wobble_U-G | | |
| | 0090 | CAAUACUCAG | 9_1-1_mismatch_C-C | | |
| | | CAGUGCGUGA | 14_1-1_mismatch_C-A | | |
| | | GUCAGCAAUC | 26_6-6_internal_loop-symmetric_GGGGAU-UAGGGG | | |
| | | UUUGCAUACU | 34_1-1_wobble_G-U | | |
| | | ACGCAGCAUU | | | |
| | | GGGAUACAGU | | | |
| | | GUGAAAAGCA | | | |
| | | GCA | | | |
| 112 | Ml_ | CCCUGGUGUG | −49_1-1_mismatch_C-A | −20 | 26 |
| | Ex- | CCCUCUGAUG | −20_6-6_internal_loop-symmetric_CAUCAU-UACUAC | | |
| | haustive_ | UUUUUUAGGG | −7_1-1_wobble_U-G | | |
| | ADAR2_ | GAUUCUACAG | −1->0_2-2_bulge-symmetric_CA-GC | | |
| | 0139 | CACGACUGAG | 4_1-1_wobble_U-G | | |
| | | CAGUGCGCUA | 13_2-2_bulge-symmetric_AC-CG | | |
| | | GUCGGCAAUC | 26_6-6_internal_loop-symmetric_GGGGAU-UAGGGG | | |
| | | UUUGCAUACU | 34_1-1_wobble_G-U | | |
| | | ACGCAGCAUU | | | |
| | | GGGAUACAGU | | | |
| | | GUGAAAAGCA | | | |
| | | GCA | | | |
| 113 | Ml_ | CCCUGGUGUG | −49_1-1_mismatch_C-A | −20 | 26 |
| | Ex | CCCUCUGAUG | −20_6-6_internal_loop-symmetric_CAUCAU-UACUAC | | |
| | haustive_ | UUUUUUAGGG | −13_1-1_mismatch_G-A | | |
| | ADAR2_ | GAUUCUACAG | −1->0_2-2_bulge-symmetric_CA-GC | | |
| | 0395 | CACGACUGAG | 4_1-1_wobble_U-G | | |
| | | CAGUGCGCUA | 13_2-2_bulge-symmetric_AC-CG | | |
| | | GUCAGCAAUA | 26_6-6_internal_loop-symmetric_GGGGAU-UAGGGG | | |
| | | UUUGCAUACU | 34_1-1_wobble_G-U | | |
| | | ACGCAGCAUU | | | |
| | | GGGAUACAGU | | | |
| | | GUGAAAAGCA | | | |
| | | GCA | | | |
| 114 | Ml_ | CCCUGGUGUG | -49_1-1_mismatch_C-A | −20 | 26 |
| | Ex- | CCCUCUGAUG | -20_6-6_internal_loop-symmetric_CAUCAU-UACUAC | | |
| | haustive_ | UUUUUUAGGG | -10_1-1_mismatch_U-C | | |
| | ADAR2_ | GAUUCUACAG | -1->0_2-2_bulge-symmetric_CA-GC | | |
| | 0453 | CAAUACUCAG | 4_1-1_wobble_U-G | | |
| | | CAGUGCGCUA | 9_1-1_mismatch_C-C | | |
| | | GUCAGCCAUC | 14_1-1_mismatch_C-A | | |
| | | UUUGCAUACU | 26_6-6_internal_loop-symmetric_GGGGAU-UAGGGG | | |
| | | ACGCAGCAUU | 34_1-1_wobble_G-U | | |
| | | GGGAUACAGU | | | |
| | | GUGAAAAGCA | | | |
| | | GCA | | | |
| 115 | Ml_ | CCCUGGUGUG | −49_1-1_mismatch_C-A | −20 | 26 |
| | Ex- | CCCUCUGAUG | −20_6-6_internal_loop-symmetric_CAUCAU-UACUAC | | |
| | haustive_ | UUUUUUAGGG | −7_1-1_wobble_U-G | | |
| | ADAR12_ | GAUUCUACAG | 0_1-1_mismatch_A-C | | |
| | 0464 | CAUGACUGAG | 2_1-1_mismatch_C-C | | |
| | | CAGUCCCGUA | 4_1-1_wobble_U-G | | |
| | | GUCGGCAAUC | 13_2-2_bulge-symmetric_AC-UG | | |
| | | UUUGCAUACU | 26_6-6_internal_loop-symmetric_GGGGAU-UAGGGG | | |
| | | ACGCAGCAUU | 34_1-1_wobble_G-U | | |
| | | GGGAUACAGU | | | |
| | | GUGAAAAGCA | | | |
| | | GCA | | | |
| 116 | Ml_ | CCCUGGUGUG | −49_1-1_mismatch_C-A | −20 | 26 |
| | Ex- | CCCUCUGAUG | −20_6-6_internal_loop-symmetric_CAUCAU-UACUAC | | |
| | haustive_ | UUUUUUAGGG | −13_1-1_mismatch_G-A | | |
| | ADAR12_ | GAUUCUACAG | 0_1-1_mismatch_A-C | | |
| | 1042 | CAAUACUCAG | 2_1-1_mismatch_C-U | | |
| | | CAGUUCCGUA | 4_1-1_wobble_U-G | | |
| | | GUCAGCAAUA | 9_1-1_mismatch_C-C | | |
| | | UUUGCAUACU | 14_1-1_mismatch_C-A | | |
| | | ACGCAGCAUU | 26_6-6_internal_loop-symmetric_GGGGAU-UAGGGG | | |
| | | GGGAUACAGU | 34_1-1_wobble_G-U | | |
| | | GUGAAAAGCA | | | |
| | | GCA | | | |
| 117 | ML_ | CCCUGGUGUG | −49_1-1_mismatch_C-A | −20 | 26 |
| | Ex- | CCCUCUGAUG | −20_6-6_internal_loop-symmetric_CAUCAU-UACUAC | | |
| | haustve_ | UUUUUUAGGG | −13_1-1_mismatch_G-A | | |
| | ADAR12_ | GAUUCUACAG | 0_1-1_mismatch_A-C | | |
| | 104 | CAUUACUCAG | 2_1-1_mismatch_C-C | | |
| | | CAGUCCCGUA | 4_1-1_wobble_U-G | | |
| | | GUCAGCAAUA | 9_1-1_mismatch_C-C | | |
| | | UUUGCAUACU | 14_1-1_mismatch_C-U | | |
| | | ACGCAGCAUU | 26_6-6_internal_loop-symmetric_GGGGAU-UAGGGG | | |
| | | GGGAUACAGU | 34_1-1_wobble_G-U | | |
| | | GUGAAAAGCA | | | |
| | | GCA | | | |
| 118 | Ml_ | CCCUGGUGUG | −49_1-1_mismatch_C-A | −20 | 26 |
| | Ex- | CCCUCUGAUG | −20_6-6_internal_loop-symmetric_CAUCAU-UACUAC | | |
| | haustive_ | UUUUUUAGGG | −9_1-1_mismatch_G-A | | |
| | ADAR12_ | GAUUCUACAG | 0_1-1_mismatch_A-C | | |
| | 1540 | CAUUACUCAG | 2_2-2_bulge-symmetric_CA-AU | | |
| | | CAAAUCCGUA | 9_1-1_mismatch_C-C | | |
| | | GUCAGAAAUC | 14_1-1_mismatch_C-U | | |
| | | UUUGCAUACU | 26_6-6_internal_loop-symmetric_GGGGAU-UAGGGG | | |
| | | ACGCAGCAUU | 34_1-1_wobble_G-U | | |
| | | GGGAUACAGU | | | |
| | | GUGAAAAGCA | | | |
| | | GCA | | | |
| 119 | Ml_ | CCCUGGUGUG | −49_1-1_mismatch_C-A | −20 | 26 |
| | Gen- | CCCUCUGAUG | −20_6-6_internal_loop-symmetric_CAUCAU-UACUAC | | |
| | erative_ | UUUUUUAGGG | −10_1-1_mismatch_U-C | | |
| | 0002 | GAUUCUAGAG | −7_1-1_wobble_U-G | | |
| | | AGGUACUGUG | −3_1-1_wobble_U-G | | |
| | | CCAUCCCGUG | 0_1-1_mismatch_A-C | | |
| | | GUCGGCCAUC | 2_1-1_mismatch_C-C | | |
| | | UUUGCAUACU | 5_1-1_mismatch_U-C | | |
| | | ACGCAGCAUU | 8_1-1_mismatch_U-U | | |
| | | GGGAUACAGU | 15_1-1_wobble_U-G | | |
| | | GUGAAAAGCA | 16_1_mismatch_G-A | | |
| | | GCA | 19_1-1_mismatch_G-G | | |
| | | | 26_6-6_internal_loop-symmetric_GGGGAU-UAGGGG | | |
| | | | 34_1-1_wobble_G-U | | |
| 120 | Ml_ | CCCUGGUGUG | −49_1-1_mismatch_C-A | −20 | 26 |
| | Gen- | CCCUCUGAUG | −20_6-6_internal_loop-symmetric_CAUCAU-UACUAC | | |
| | erative_ | UUUUUUAGGG | −7_1-1_wobble_U-G | | |
| | 0013 | GAUUCUAGAG | −3_1-1_wobble_U-G | | |
| | | CAGGACGGUG | 0_1-1_mismatch_A-C | | |
| | | CAGUCCCGUG | 2_1-1_mismatch_C-C | | |
| | | GUCGGCAAUC | 4_1-1_wobble_U-G | | |
| | | UUUGCAUACU | 8_1-1_mismatch_U-U | | |
| | | ACGCAGCAUU | 10_1-1_mismatch_A-G | | |
| | | GGGAUACAGU | 13_1-1_mismatch_A-G | | |
| | | GUGAAAAGCA | 19_1-1_mismatch_G-G | | |
| | | GCA | 26_6-6_internal_loop-symmetric_GGGGAU-UAGGGG | | |
| | | | 34_1-1_wobble_G-U | | |
| 121 | Ml_ | CCCUGGUGUG | −49_1-1_mismatch_C-A | −20 | 26 |
| | Gen- | CCCUCUGAUG | −20_6-6_internal_loop-symmetric_CAUCAU-UACUAC | | |
| | erative_ | UUUUUUAGGG | −10_2-2_bulge-symmetric_UU-CU | | |
| | 0016 | GAUUCUACAG | −8_1-1_mismatch_C-C | | |
| | | GAGGACUGGG | −7_1-1_wobble_U-G | | |
| | | CAGUCCCGUG | −3_1_wobble_U-G | | |
| | | GUCGCCCUUC | 0_1-1_mismatch_A-C | | |
| | | UUUGCAUACU | 2_1-1_mismatch_C-C | | |
| | | ACGCAGCAUU | 4_1-1_wobble_U-G | | |
| | | GGGAUACAGU | 8_1-1_wobble_U-G | | |
| | | GUGAAAAGCA | 13_1-1_mismatch_A-G | | |
| | | GCA | 16_1-1_mismatch_G-G | | |
| | | | 26_6-6_internal_loop-symmetric_GGGGAU-UAGGGG | | |
| | | | 34_1-1_wobble_G-U | | |
| 122 | Ml_ | CCCUGGUGUG | −49_1-1_mismatch_C-A | −20 | 26 |
| | Gen- | CCCUCUGAUG | −20_6-6_internal_loop-symmetric_CAUCAU-UACUAC | | |
| | erative_ | UUUUUUAGGG | −10_1-1_mismatch_U-C | | |
| | 0043 | GAUUCUACAG | −9_1-1_wobble_G-U | | |
| | | CGGUACUGUG | −7_1-1_wobble_U-G | | |
| | | CAAAUCCGUG | −3_1-1_wobble_U-G | | |
| | | GUCGGUCAUC | 0_1-1_mismatch_A-C | | |
| | | UUUGCAUACU | 2_2-2_bulge-symmetric_CA-AU | | |
| | | ACGCAGCAUU | 8_1-1_mismatch_U-U | | |
| | | GGGAUACAGU | 15_1-1_wobble_U-G | | |
| | | GUGAAAAGCA | 26_6-6_internal_loop-symmetric_GGGGAU-UAGGGG | | |
| | | GCA | 34_1-1_wobble_G-U | | |
| 123 | Ml_ | CCCUGGUGUG | −49_1-1_mismatch_C-A | −20 | 26 |
| | Gen- | CCCUCUGAUG | −20_6-6_internal_loop-symmetric_CAUCAU-UACUAC | | |
| | erative_ | UUUUUUAGGG | −10_1-1_mismatch_U-C | | |
| | 0058 | GAUUCUACGG | −7_1-1_wobble_U-G | | |
| | | CAGGACUGGG | −3_1-1_wobble_U-G | | |
| | | GAAAUCCGUG | 0_1-1_mismatch_A-C | | |
| | | GUCGGCCAUC | 2_2-2_bulge-symmetric_CA-AU | | |
| | | UUUGCAUACU | 6_1-1_mismatch_G-G | | |
| | | ACGCAGCAUU | 8_1-1_wobble_U-G | | |
| | | GGGAUACAGU | 13_1-1_mismatch_A-G | | |
| | | GUGAAAAGCA | 18_1-1_wobble_U-G | | |
| | | GCA | 26_6-6_internal_loop-symmetric_GGGGAU-UAGGGG | | |
| | | | 34_1-1_wobble_G-U | | |
| 124 | Ml_ | CCCUGGUGUG | −49_1-1_mismatch_C-A | −20 | 26 |
| | Gen- | CCCUCUGAUG | −20_6-6_internal_loop-symmetric_CAUCAU-UACUAC | | |
| | erative_ | UUUUUUAGGG | −10_1-1_mismatch_U-C | | |
| | 0071 | GAUUCUACAG | −8_1-1_mismatch_C-U | | |
| | | CGGUACUGCG | −7_1-1_wobble_U-G | | |
| | | CAGUCCCGUG | −6_1-1_mismatch_G-G | | |
| | | GUGGUCCAUC | −3_1-1_wobble_U-G | | |
| | | UUUGCAUACU | 0_1-1_mismatch_A-C | | |
| | | ACGCAGCAUU | 2_1-1_mismatch_C-C | | |
| | | GGGAUACAGU | 4_1-1_wobble_U-G | | |
| | | GUGAAAAGCA | 8_1-1_mismatch_U-C | | |
| | | GCA | 15_1-1_wobble_U-G | | |
| | | | 26_6-6_internal_loop-symmetric_GGGGAU-UAGGGG | | |
| | | | 34_1-1_wobble_G-U | | |
| 125 | Ml_ | CCCUGGUGUG | −49_1-1_mismatch_C-A | −20 | 26 |
| | Gen- | CCCUCUGAUG | −20_6-6_internal_loop-symmetric_CAUCAU-UACUAC | | |
| | erative_ | UUUUUUAGGG | −7_1-1_wobble_U-G | | |
| | 0130 | GAUUCUAUAG | −3_1-1_wobble_U-G | | |
| | | CAGGAAUGAG | 0_1-1_mismatch_A-C | | |
| | | GAAAUCCGUG | 2_2-2_bulge-symmetric_CA-AU | | |
| | | GUCGGCAAUC | 6_1-1_mismatch_G-G | | |
| | | UUUGCAUACU | 11_3-3_bulge-symmetric_GUA-GAA | | |
| | | ACGCAGCAUU | 19_1-1_wobble_G-U | | |
| | | GGGAUACAGU | 26_6-6_internal_loop-symmetric_GGGGAU-UAGGGG | | |
| | | GUGAAAAGCA | 34_1-1_wobble_G-U | | |
| | | GCA | | | |
| 126 | Ml_ | CCCUGGUGUG | −49_1-1_mismatch_C-A | −20 | 26 |
| | Gen- | CCCUCUGAUG | −20_6-6_internal_loop-symmetric_CAUCAU-UACUAC | | |
| | erative_ | UUUUUUAGGG | −9_2-2_bulge-symmetric_UG-AU | | |
| | 0156 | GAUUCUACAG | −7_1-1_wobble_U-G | | |
| | | CAGUACUCAG | −3_1-1_wobble_U-G | | |
| | | GAAAUCCGUG | 0_1-1_mismatch_A-C | | |
| | | GUCGGAUAUC | 2_2-2_bulge-symmetric_CA-AU | | |
| | | UUUGCAUACU | 6_1-1_mismatch_G-G | | |
| | | ACGCAGCAUU | 9_1-1_mismatch_C-C | | |
| | | GGGAUACAGU | 26_6-6_internal_loop-symmetric_GGGGAU-UAGGGG | | |
| | | GUGAAAAGCA | 34_1-1_wobble_G-U | | |
| | | GCA | | | |
| 127 | Ml_ | CCCUGGUGUG | −49_1-1_mismatch_C-A | −20 | 26 |
| | Gen- | CCCUCUGAUG | −20_6-6_internal_loop-symmetric_CAUCAU-UACUAC | | |
| | erative_ | UUUUUUAGGG | −7_1-1_wobble_U-G | | |
| | 0176 | GAUUCUAAAG | −3_1-1_wobble_U-G | | |
| | | CAGGAGUGAG | 0->3_4-4_bulge-symmetric_AGCA-AUCC | | |
| | | CAGAUCCGUG | 4_1-1_wobble_U-G | | |
| | | GUCGGCAAUC | 11_1-1_mismatch_G-G | | |
| | | UUUGCAUACU | 13_1-1_mismatch_A-G | | |
| | | ACGCAGCAUU | 19_1-1_mismatch_G-A | | |
| | | GGGAUACAGU | 26_6-6_internal_loop-symmetric_GGGGAU-UAGGGG | | |
| | | GUGAAAAGCA | 34_1-1_wobble_G-U | | |
| | | GCA | | | |
| 128 | Ml_ | CCCUGGUGUG | −49_1-1_mismatch_C-A | −20 | 26 |
| | Gen- | CCCUCUGAUG | −20_6-6_internal_loop-symmetric_CAUCAU-UACUAC | | |
| | erative_ | UUUUUUAGGG | −8_2-2_bulge-symmetric_GC-UA | | |
| | 0218 | GAUUCUACAG | −3_1-1_wobble_U-G | | |
| | | GUGUACUGUG | 0_1-1_mismatch_A-C | | |
| | | AAAUCCCGUG | 2_1-1_mismatch_C-C | | |
| | | GUCAUAAAUC | 6_1-1_mismatch_G-A | | |
| | | UUUGCAUACU | 8_1-1_mismatch_U-U | | |
| | | ACGCAGCAUU | 15_2-2_bulge-symmetric_UG-GU | | |
| | | GGGAUACAGU | 26_6-6_internal_loop-symmetric_GGGGAU-UAGGGG | | |
| | | GUGAAAAGCA | 34_1-1_wobble_G-U | | |
| | | GCA | | | |
| 129 | Ml_ | CCCUGGUGUG | −49_1-1_mismatch_C-A | −20 | 26 |
| | Gen- | CCCUCUGAUG | −20_6-6_internal_loop-symmetric_CAUCAU-UACUAC | | |
| | erative_ | UUUUUUAGGG | −8_2-2_bulge-symmetric_GC-UA | | |
| | 0274 | GAUUCUACAG | −7_1-1_wobble_U-G | | |
| | | CAGUACUGUC | −3_1-1_wobble_U-G | | |
| | | CAGUCCCGUG | 0_1-1_mismatch_A-C | | |
| | | GUCGUAAAUC | 2_1-1_mismatch_C-C | | |
| | | UUUGCAUACU | 4_1-1_wobble_U-G | | |
| | | ACGCAGCAUU | 7_2-2_bulge-symmetric_CU-UC | | |
| | | GGGAUACAGU | 26_6-6_internal_loop-symmetric_GGGGAU-UAGGGG | | |
| | | GUGAAAAGCA | 34_1-1_wobble_G-U | | |
| | | GCA | | | |
| 130 | ML_ | CCCUGGUGUG | −49_1-1_mismatch_C-A | −20 | 26 |
| | Gen- | CCCUCUGAUG | −20_6-6_internal_loop-symmetric_CAUCAU-UACUAC | | |
| | erative_ | UUUUUUAGGG | −10_1-1_mismatch_U-U | | |
| | 0325 | GAUUCUACAU | −7_1-1_wobble_U-G | | |
| | | CAGGACUGAG | −3_1-1_wobble_U-G | | |
| | | CUUUUCCGAG | −2->5_8-8_internal_loop-symmetric_ACAGCAUU- | | |
| | | GUCGGCUAUC | UUUUCCGA | | |
| | | UUUGCAUACU | 13_1-1_mismatch_A-G | | |
| | | ACGCAGCAUU | 17_1-1_mismatch_C-U | | |
| | | GGGAUACAGU | 26_6-6_internal_loop-symmetric_GGGGAU-UAGGGG | | |
| | | GUGAAAAGCA | 34_1-1_wobble_G-U | | |
| | | GCA | | | |
| 131 | Ml_ | CCCUGGUGUG | −49_1-1_mismatch_C-A | −20 | 26 |
| | Gen- | CCCUCUGAUG | −20_6-6_internal_loop-symmetric_CAUCAU-UACUAC | | |
| | erative_ | UUUUUUAGGG | −7_1-1_wobble_U-G | | |
| | 0332 | GAUUCUACAU | 0_1-1_mismatch_A-C | | |
| | | CAGUACUGGG | 2_1-1_mismatch_C-U | | |
| | | CAGUUCCGUA | 4_1-1_wobble_U-G | | |
| | | GUCGGCAAUC | 8_1-1_wobble_U-G | | |
| | | UUUGCAUACU | 17_1-1_mismatch_C-U | | |
| | | ACGCAGCAUU | 26_6-6_internal_loop-symmetric_GGGGAU-UAGGGG | | |
| | | GGGAUACAGU | 34_1-1_wobble_G-U | | |
| | | GUGAAAAGCA | | | |
| | | GCA | | | |
| 132 | Ml_ | CCCUGGUGUG | −49_1-1_mismatch_C-A | −20 | 26 |
| | Gen- | CCCUCUGAUG | −20_6-6_internal_loop-symmetric_CAUCAU-UACUAC | | |
| | erative_ | UUUUUUAGGG | −11_1-1_mismatch_U-C | | |
| | 0559 | GAUUCUACAG | −7_1-1_wobble_U-G | | |
| | | CAGUACUGGG | −1->0_2-2_bulge-symmetric_CA-GC | | |
| | | CAGUGCGCUA | 4_1-1_wobble_U-G | | |
| | | GUCGGCACUC | 8_1-1_wobble_U-G | | |
| | | UUUGCAUACU | 26_6-6_internal_loop-symmetric_GGGGAU-UAGGGG | | |
| | | ACGCAGCAUU | 34_1-1_wobble_G-U | | |
| | | GGGAUACAGU | | | |
| | | GUGAAAAGCA | | | |
| | | GCA | | | |
| 133 | ML_ | CCCUGGUGUG | −49_1-1_mismatch_C-A | −20 | 26 |
| | Gen- | CCCUCUGAUG | −20_6-6_internal_loop-symmetric_CAUCAU-UACUAC | | |
| | erative_ | UUUUUUAGGG | −6_1-1_mismatch_G-A | | |
| | 0639 | GAUUCUACAG | −3->2_6-6_internal_loop-symmetric_UACAGC- | | |
| | | CAAUACUCAC | CCCGAU | | |
| | | CAGUCCCGAU | 4_1-1_wobble_U-G | | |
| | | GUAAGCAAUC | 7_3-3_bulge-symmetric_CUC-CAC | | |
| | | UUUGCAUACU | 14_1-1_mismatch_C-A | | |
| | | ACGCAGCAUU | 26_6-6_internal_loop-symmetric_GGGGAU-UAGGGG | | |
| | | GGGAUACAGU | 34_1-1_wobble_G-U | | |
| | | GUGAAAAGCA | | | |
| | | GCA | | | |
| 134 | Ml_ | CCCUGGUGUG | −49_1-1_mismatch_C-A | −20 | 26 |
| | Gen- | CCCUCUGAUG | −20_6-6_internal_loop-symmetric_CAUCAU-UACUAC | | |
| | erative_ | UUUUUUAGGG | −7_1-1_wobble_U-G | | |
| | 0643 | GAUUCUACGG | −3->6_10-10_internal_loop- | | |
| | | CAGUACUGUG | symmetric_UACAGCAUUG-AAAUCCCGAU | | |
| | | AAAUCCCGAU | 8_1-1_mismatch_U-U | | |
| | | GUCGGCAAUC | 18_1-1_wobble_U-G | | |
| | | UUUGCAUACU | 26_6-6_internal_loop-symmetric_GGGGAU-UAGGGG | | |
| | | ACGCAGCAUU | 34_1-1_wobble_G-U | | |
| | | GGGAUACAGU | | | |
| | | GUGAAAAGCA | | | |
| | | GCA | | | |
| 135 | Ml_ | CCCUGGUGUG | −49_1-1_mismatch_C-A | −20 | 26 |
| | Gen- | CCCUCUGAUG | −20_6-6_internal_loop-symmetric_CAUCAU-UACUAC | | |
| | erative_ | UUUUUUAGGG | −7_0-1_bulge-asymmetric_-G | | |
| | 0644 | GAUUCUACCG | −6_1-1_wobble_G-U | | |
| | | CAGGACGGAG | −2->5_8-7_internal_loop-asymmetric_ACAGCAUU- | | |
| | | CUAUCCCGAG | UAUCCCG | | |
| | | UUAGGCAAUC | 10_1-1_mismatch_A-G | | |
| | | UUUGCAUACU | 13_1-1_mismatch_A-G | | |
| | | ACGCAGCAUU | 18_1-1_mismatch_U-C | | |
| | | GGGAUACAGU | 26_6-6_internal_loop-symmetric_GGGGAU-UAGGGG | | |
| | | GUGAAAAGCA | 34_1-1_wobble_G-U | | |
| | | GCA | | | |
| 136 | Ml_ | CCCUGGUGUG | −49_1-1_mismatch_C-A | −20 | 26 |
| | Gen- | CCCUCUGAUG | −20_6-6_internal_loop-symmetric_CAUCAU-UACUAC | | |
| | erative_ | UUUUUUAGGG | −14_2-2_bulge-symmetric_AA-GA | | |
| | 0690 | GAUUCUACGG | −9_4-4_bulge-symmetric_AUUG-ACCC | | |
| | | CAUGACUGAU | −7_1-1_wobble_U-G | | |
| | | CAGUCCCGUG | −3_1-1_wobble_U-G | | |
| | | GUCGGACCCC | 0_1-1_mismatch_A-C | | |
| | | GAUGCAUACU | 2_1-1_mismatch_C-C | | |
| | | ACGCAGCAUU | 4_1-1_wobble_U-G | | |
| | | GGGAUACAGU | 7_1-1_mismatch_C-U | | |
| | | GUGAAAAGCA | 13_2-2_bulge-symmetric_AC-UG | | |
| | | GCA | 18_1-1_wobble_U-G | | |
| | | | 26_6-6_internal_loop-symmetric_GGGGAU-UAGGGG | | |
| | | | 34_1-1_wobble_G-U | | |
| 137 | Ml_ | CCCUGGUGUG | −49_1-1_mismatch_C-A | −20 | 26 |
| | Gen- | CCCUCUGAUG | −20_6-6_internal_loop-symmetric_CAUCAU-UACUAC | | |
| | erative_ | UUUUUUAGGG | −9_7-7_internal_loop-symmetric_AAGAUUG- | | |
| | 0699 | GAUUCUACGG | ACCCCCG | | |
| | | CAAGACUGGG | −7_1-1_wobble_U-G | | |
| | | CAGUCCCGUG | −3_1-1_wobble_U-G | | |
| | | GUCGGACCCC | 0_1-1_mismatch_A-C | | |
| | | CGUGCAUACU | 2_1-1_mismatch_C-C | | |
| | | ACGCAGCAUU | 4_1-1_wobble_U-G | | |
| | | GGGAUACAGU | 8_1-1_wobble_U-G | | |
| | | GUGAAAAGCA | 13_2-2_bulge-symmetric_AC-AG | | |
| | | GCA | 18_1-1_wobble_U-G | | |
| | | | 26_6-6_internal_loop-symmetric_GGGGAU-UAGGGG | | |
| | | | 34_1-1_wobble_G-U | | |
| 138 | Ml_ | CCCUGGUGUG | −49_1-1_mismatch_C-A | −20 | 26 |
| | Gen- | CCCUCUGAUG | −20_6-6_internal_loop-symmetric_CAUCAU-UACUAC | | |
| | erative_ | UUUUUUAGGG | −14_2-2_bulge-symmetric_AA-CG | | |
| | 0701 | GAUUCUACGG | −9_3-3_bulge-symmetric_UUG-ACC | | |
| | | CAGGACUGGG | −7_1-1_wobble_U-G | | |
| | | CUUUCCCGUG | −3_1-1_wobble_U-G | | |
| | | GUCGGACCUC | 0_1-1_mismatch_A-C | | |
| | | CGUGCAUACU | 2_1-1_mismatch_C-C | | |
| | | ACGCAGCAUU | 4_2-2_bulge-symmetric_UU-UU | | |
| | | GGGAUACAGU | 8_1-1_wobble_U-G | | |
| | | GUGAAAAGCA | 13_1-1_mismatch_A-G | | |
| | | GCA | 18_1-1_wobble_U-G | | |
| | | | 26_6-6_internal_loop-symmetric_GGGGAU-UAGGGG | | |
| | | | 34_1-1_wobble_G-U | | |
| 139 | Ml_ | CCCUGGUGUG | −49_1-1_mismatch_C-A | −20 | |
| | Gen- | CCCUCUGAUG | −20_6-6_internal_loop-symmetric_CAUCAU-UACUAC | | |
| | erative_ | UUUUUUAGGG | | | |
| | 0703 | GAUUCUACGG | | | |
| | | UAGGACUGAU | | | |
| | | CAGUCCCGUG | | | |
| | | GUCGAACCAC | | | |
| | | GUUGCAUACU | | | |
| | | ACGCAGCAUU | | | |
| | | GGGAUACAGU | | | |
| | | GUGAAAAGCA | | | |
| | | GCA | | | |
| 140 | Ml_ | CCCUGGUGUG | −49_1-1_mismatch_C-A | −20 | 26 |
| | Gen- | CCCUCUGAUG | −20_6-6_internal_loop-symmetric_CAUCAU-UACUAC | | |
| | erative_ | UUUUUUAGGG | −6_9-9_internal_loop-symmetric_AGAUUGCUG- | | |
| | 0719 | GAUUCUACGG | GGAACCCCG | | |
| | | CAAGACUGAG | −3_1-1_wobble_U-G | | |
| | | CAGUCCCGUG | 0_1-1_mismatch_A-C | | |
| | | GUGGAACCCC | 2_1-1_mismatch_C-C | | |
| | | GUUGCAUACU | 4_1-1_wobble_U-G | | |
| | | ACGCAGCAUU | 13_2-2_bulge-symmetric_AC-AG | | |
| | | GGGAUACAGU | 18_1-1_wobble_U-G | | |
| | | GUGAAAAGCA | 26_6-6_internal_loop-symmetric_GGGGAU-UAGGGG | | |
| | | GCA | 34_1-1_wobble_G-U | | |
| 141 | Ml_ | CCCUGGUGUG | −49_1-1_mismatch_C-A | −20 | 26 |
| | Gen- | CCCUCUGAUG | −20_6-6_internal_loop-symmetric_CAUCAU-UACUAC | | |
| | erative_ | UUUUUUAGGG | −11_4-5_internal_loop-asymmetric_AGAU-CCCCG | | |
| | 0728 | GAUCCUACGG | −7_1-1_wobble_U-G | | |
| | | CAGUACUGAU | −6_1-0_bulge-asymmetric_G- | | |
| | | CAAAUCCGUG | −3_1-1_wobble_U-G | | |
| | | GUGGCACCCC | 0_1-1_mismatch_A-C | | |
| | | GUUGCAUACU | 2_2-2_bulge-symmetric_CA-AU | | |
| | | ACGCAGCAUU | 7_1-1_mismatch_C-U | | |
| | | GGGAUACAGU | 18_1-1_wobble_U-G | | |
| | | GUGAAAAGCA | 23_1-1_mismatch_A-C | | |
| | | GCA | 26_6-6_internal_loop-symmetric_GGGGAU-UAGGGG | | |
| | | | 34_1-1_wobble_G-U | | |
| 142 | Ml_ | CCCUGGUGUG | −49_1-1_mismatch_C-A | −20 | 26 |
| | Gen- | CCCUCUGAUG | −20_6-6_internal_loop-symmetric_CAUCAU-UACUAC | | |
| | erative_ | UUUUUUAGGG | −14_2-2_bulge-symmetric_AA-CG | | |
| | 0732 | GAUUCUACAG | −11_1-2_bulge-asymmetric_U-CC | | |
| | | CAGGACUGAC | −7_1-1_wobble_U-G | | |
| | | CAGUUCCGUG | −6_1-0_bulge-asymmetric_G- | | |
| | | GUGGCACCUC | −3_1-1_wobble_U-G | | |
| | | CGUGCAUACU | 0_1-1_mismatch_A-C | | |
| | | ACGCAGCAUU | 2_1-1_mismatch_C-U | | |
| | | GGGAUACAGU | 4_1-1_wobble_U-G | | |
| | | GUGAAAAGCA | 7_1-1_mismatch_C-C | | |
| | | GCA | 13_1-1_mismatch_A-G | | |
| | | | 26_6-6_internal_loop-symmetric_GGGGAU-UAGGGG | | |
| | | | 34_1-1_wobble_G-U | | |
| 143 | Ml_ | CCCUGGUGUG | -49_1-1_mismatch_C-A | −20 | 26 |
| | Gen- | CCCUCUGAUG | -20_6-6_internal_loop-symmetric_CAUCAU-UACUAC | | |
| | erative_ | UUUUUUAGGG | -11_4-5_internal_loop-asymmetric_AGAU-CCCCG | | |
| | 0733 | GAUUCUACGG | -7_1-1_wobble_U-G | | |
| | | CAGUACUGAG | -6_1-0_bulge-asymmetric_G- | | |
| | | AAAUCCCGUG | -3_1-1_wobble_U-G | | |
| | | GUGGCACCCC | 0_1-1_mismatch_A-C | | |
| | | GUUGCAUACU | 2_1-1_mismatch_C-C | | |
| | | ACGCAGCAUU | 6_1-1_mismatch_G-A | | |
| | | GGGAUACAGU | 18_1-1_wobble_U-G | | |
| | | GUGAAAAGCA | 26_6-6_internal_loop-symmetric_GGGGAU-UAGGGG | | |
| | | GCA | 34_1-1_wobble_G-U | | |
| 144 | Ml_ | CCCUGGUGUG | −49_1-1_mismatch_C-A | −20 | 26 |
| | Gen- | CCCUCUGAUG | −20_6-6_internal_loop-symmetric_CAUCAU-UACUAC | | |
| | erative_ | UUUUUUAGGG | −14_2-2_bulge-symmetric_AA-CG | | |
| | 0742 | GAUUCUACAG | −11_1-2_bulge-asymmetric_U-CC | | |
| | | CAGUCCUGAC | −9_1-1_wobble_G-U | | |
| | | CAGUCCCGUG | −7_1-0_bulge-asymmetric_U- | | |
| | | GUCGUACCUC | −3_1-1_wobble_U-G | | |
| | | CGUGCAUACU | 0_1-1_mismatch_A-C | | |
| | | ACGCAGCAUU | 2_1-1_mismatch_C-C | | |
| | | GGGAUACAGU | 4_1-1_wobble_U-G | | |
| | | GUGAAAAGCA | 7_1-1_mismatch_C-C | | |
| | | GCA | 12_1-1_mismatch_U-C | | |
| | | | 26_6-6_internal_loop-symmetric_GGGGAU-UAGGGG | | |
| | | | 34_1-1_wobble_G-U | | |
| 145 | Ml_ | CCCUGGUGUG | −49_1-1_mismatch_C-A | −20 | |
| | Gen- | CCCUCUGAUG | −20_6-6_internal_loop-symmetric_CAUCAU-UACUAC | | |
| | erative_ | UUUUUUAGGG | | | |
| | 0743 | GCUUCUACAG | | | |
| | | CAGGACUGAU | | | |
| | | CAAAUCCGUG | | | |
| | | GUGGAACCCC | | | |
| | | GUUGCAUACU | | | |
| | | ACGCAGCAUU | | | |
| | | GGGAUACAGU | | | |
| | | GUGAAAAGCA | | | |
| | | GCA | | | |
| 146 | Ml_ | CCCUGGUGUG | −49_1-1_mismatch_C-A | | |
| | Gen- | CCCUCUGAUG | | | |
| | erative_ | UUUUUUAGGG | | | |
| | 0745 | GAUUCUACGG | | | |
| | | CAGUACUGAG | | | |
| | | ACAUCCCGUG | | | |
| | | GUGGCACCCC | | | |
| | | CGUGCAUACU | | | |
| | | ACGCAGCAUU | | | |
| | | GGGAUACAGU | | | |
| | | GUGAAAAGCA | | | |
| | | GCA | | | |
| 147 | Ml_ | CCCUGGUGUG | −49_1-1_mismatch_C-A | −20 | 26 |
| | Gen- | CCCUCUGAUG | −20_6-6_internal_loop-symmetric_CAUCAU-UACUAC | | |
| | erative_ | UUUUUUAGGG | −8_1-1_mismatch_C-U | | |
| | 0766 | GAUUCUACCG | −7_1-1_wobble_U-G | | |
| | | CAGUACUCAC | −3_1-0_bulge-asymmetric_U- | | |
| | | CAGUCGCCGU | 0_1-1_mismatch_A-C | | |
| | | GUCGUCAAUC | 2_0-1_bulge-asymmetric_-C | | |
| | | UUUGCAUACU | 4_1-1_wobble_U-G | | |
| | | ACGCAGCAUU | 7_3-3_bulge-symmetric_CUC-CAC | | |
| | | GGGAUACAGU | 18_1-1_mismatch_U-C | | |
| | | GUGAAAAGCA | 26_6-6_internal_loop-symmetric_GGGGAU-UAGGGG | | |
| | | GCA | 34_1-1_wobble_G-U | | |
| 148 | Ml_ | CCCUGGUGUG | −49_1-1_mismatch_C-A | −20 | 26 |
| | Gen- | CCCUCUGAUG | −20_6-6_internal_loop-symmetric_CAUCAU-UACUAC | | |
| | erative_ | UUUUUUAGGG | −7_1-1_wobble_U-G | | |
| | 0769 | GAUUCUACCG | −3_1-0_bulge-asymmetric_U- | | |
| | | CAGUACUCAC | 0_1-1_mismatch_A-C | | |
| | | CAGUCGCCGU | 2_0-1_bulge-asymmetric_-C | | |
| | | GUCGGCAAUC | 4_1-1_wobble_U-G | | |
| | | UUUGCAUACU | 7_3-3_bulge-symmetric_CUC-CAC | | |
| | | ACGCAGCAUU | 18_1-1_mismatch_U-C | | |
| | | GGGAUACAGU | 26_6-6_internal_loop-symmetric_GGGGAU-UAGGGG | | |
| | | GUGAAAAGCA | 34_1-1_wobble_G-U | | |
| | | GCA | | | |

The sequences for the engineered guides used as comparators in FIG. 7A-FIG. 7C is provided below in TABLE 4. While the engineered guide RNA sequences in TABLE 4 are provided as DNA sequences with a T substituted for each U, the corresponding RNA sequences are also encompassed herein.

TABLE 4
Comparator LRRK2 Engineered Guide RNA Sequences

| SEQ | | | | | Right |
|---|---|---|---|---|---|
| ID | | | Structural Features | Left Barbell | Barbell |
| NO | Guide Name | Sequence | (target/guide) | Position | Position |
| 149 | A-C 0.113.57 | CCCTGGTGTGCCCTCT | −20_6-6_internal_loop- | −20 | 26 |
| | (−20, +26) | GATGTTTTTTAGGGG | symmetric_CAUCAU- | | |
| | | ATTCTACAGCAGTAC | UACUAC, 0_1- | | |
| | | TGAGCAATGCCGTAG | 1_mismatch_A-C, 26_6- | | |
| | | TCAGCAATCTTTGCA | 6_internal_loop- | | |
| | | TACTACGCAGCATTG | symmetric_GGGGAU- | | |
| | | GGATACAGTGTGAAA | UAGGGG | | |
| | | AGCAGCA | | | |
| 150 | 1976 | CCCTGGTGTGCCCTCT | −20_6-6_internal_loop- | −20 | 26 |
| | 0.113.57 (−20, | GATGTTTTTTAGGGG | symmetric_CAUCAU- | | |
| | 26) | ATTCTACAACAGTAC | UACUAC, −8_1- | | |
| | | TGAGCTATCCCGAAT | 1_mismatch_C-A, −4- | | |
| | | TCAACAATCTTTGCA | >5_10−10_internal_loop- | | |
| | | TACTACGCAGCATTG | symmetric_CUACAGCAUU- | | |
| | | GGATACAGTGTGAAA | UAUCCCGAAU, 17_1- | | |
| | | AGCAGCA | 1_mismatch_C-A, 26_6- | | |
| | | | 6_internal_loop- | | |
| | | | symmetric_GGGGAU- | | |
| | | | UAGGGG | | |
| 151 | 610 0.113.57 | CCCTGGTGTGCCCTCT | −20_6-6_internal_loop- | −20 | 26 |
| | (−20,26) | GATGTTTTTTAGGGG | symmetric_CAUCAU- | | |
| | | ATTCTACATCTGTAGT | UACUAC, −4 1- | | |
| | | GAGCAATTCCGTGCT | 1_mismatch_C-C, −3_1- | | |
| | | CAGCAATCTTTGCAT | 1_wobble_U-G, 0_1- | | |
| | | ACTACGCAGCATTGG | 1_mismatch_A-C, 2_1- | | |
| | | GATACAGTGTGAAAA | 1_mismatch_C-U, 11_1- | | |
| | | GCAGCA | 1_mismatch_G-G, 15_1- | | |
| | | | 1_mismatch_U-U, 17_1- | | |
| | | | 1_mismatch_C-U, 26_6- | | |
| | | | 6_internal_loop- | | |
| | | | symmetric_GGGGAU- | | |
| | | | UAGGGG | | |

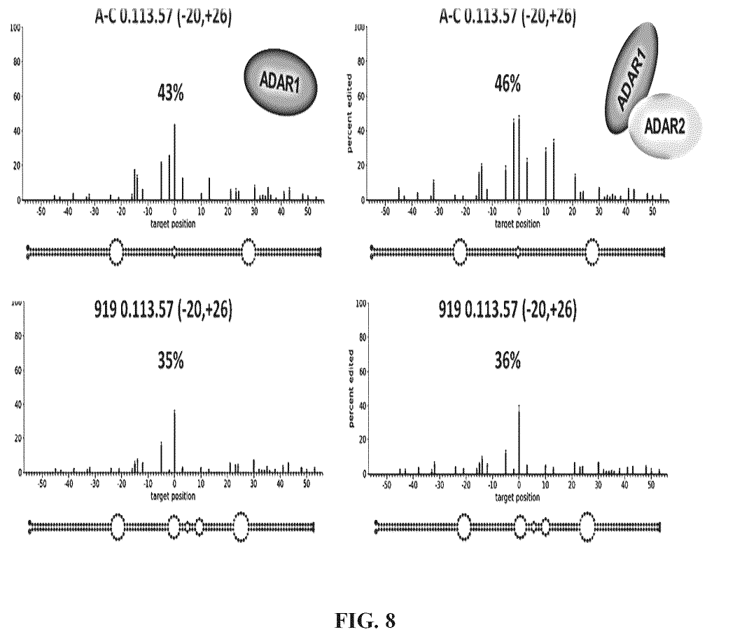

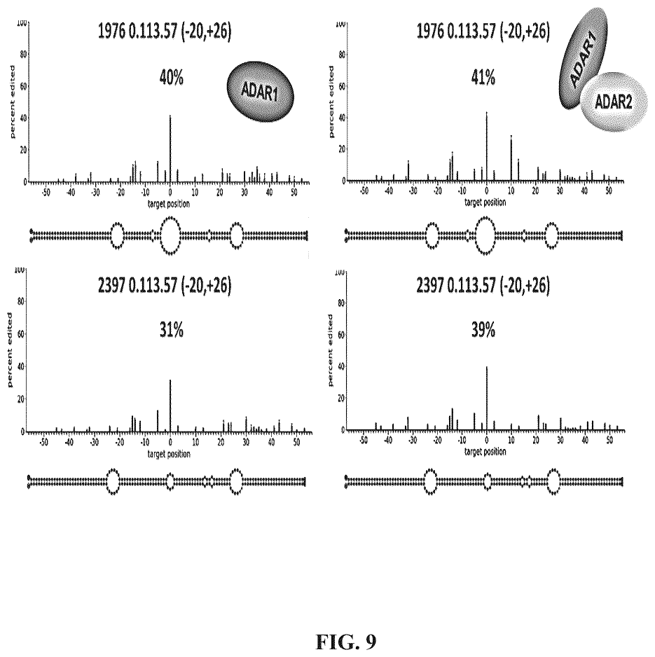

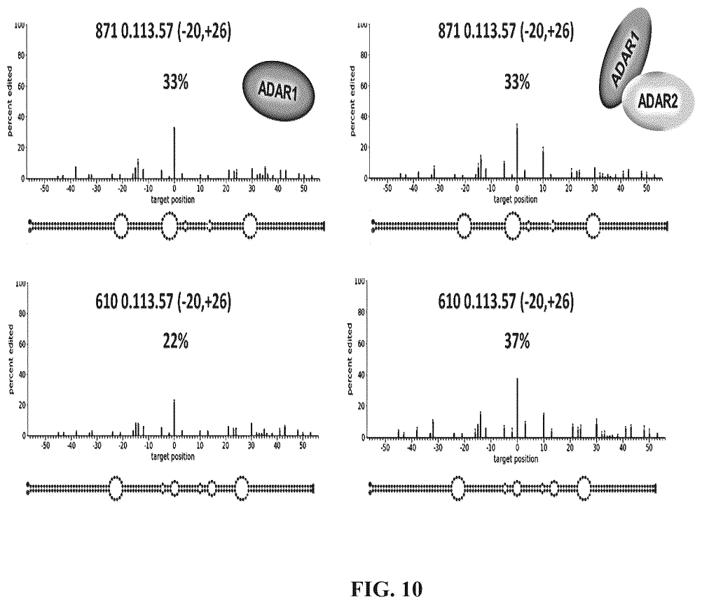

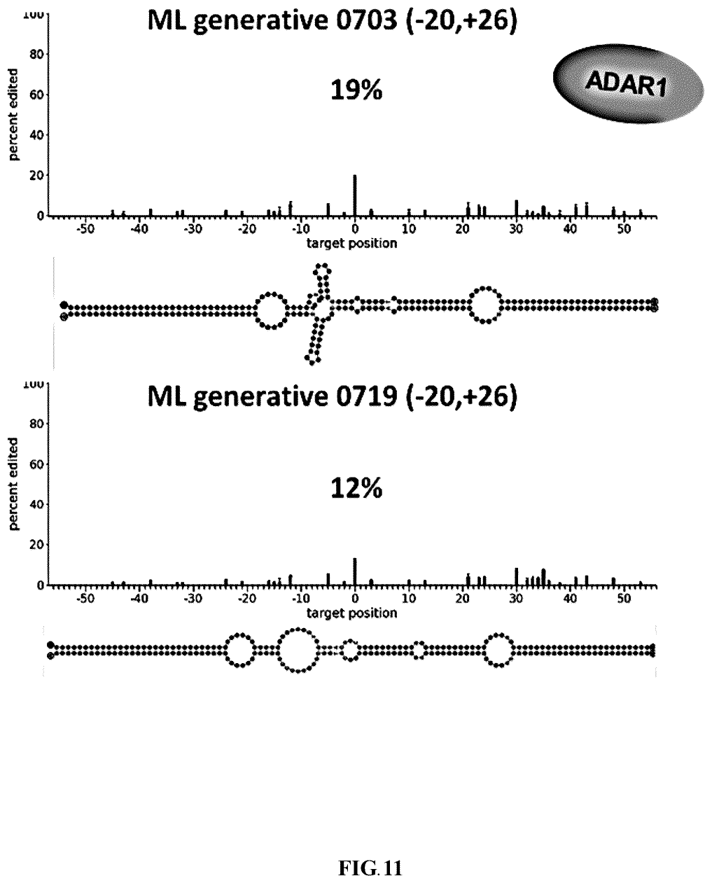

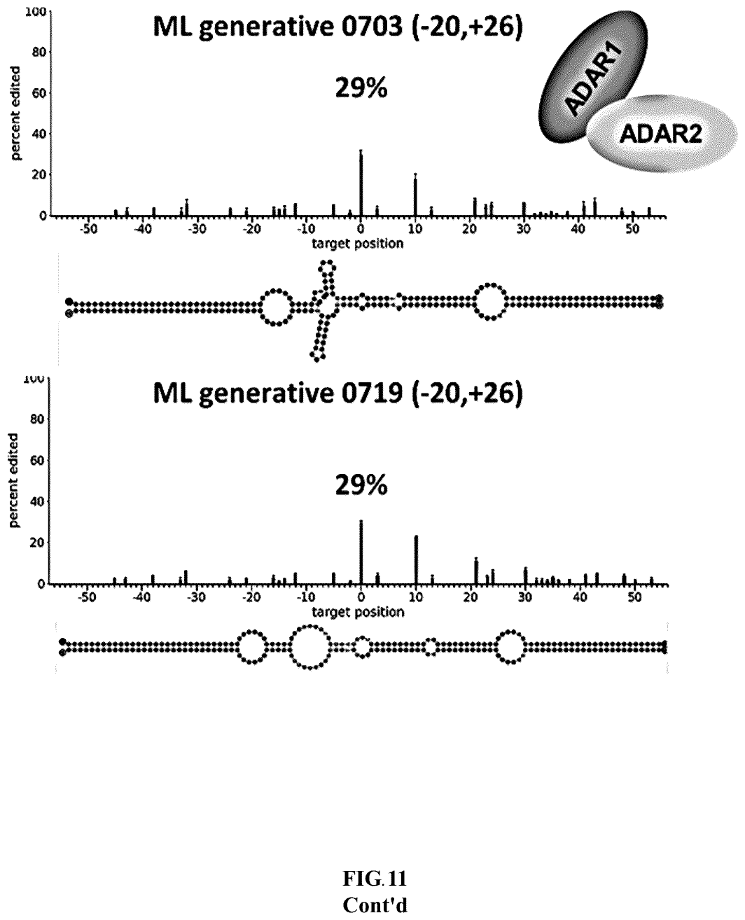

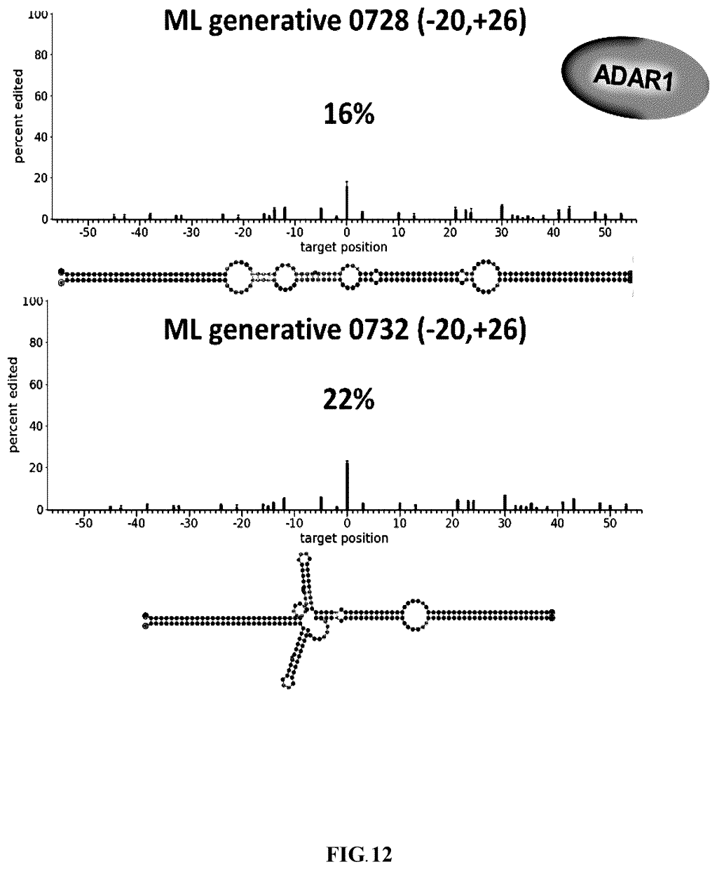

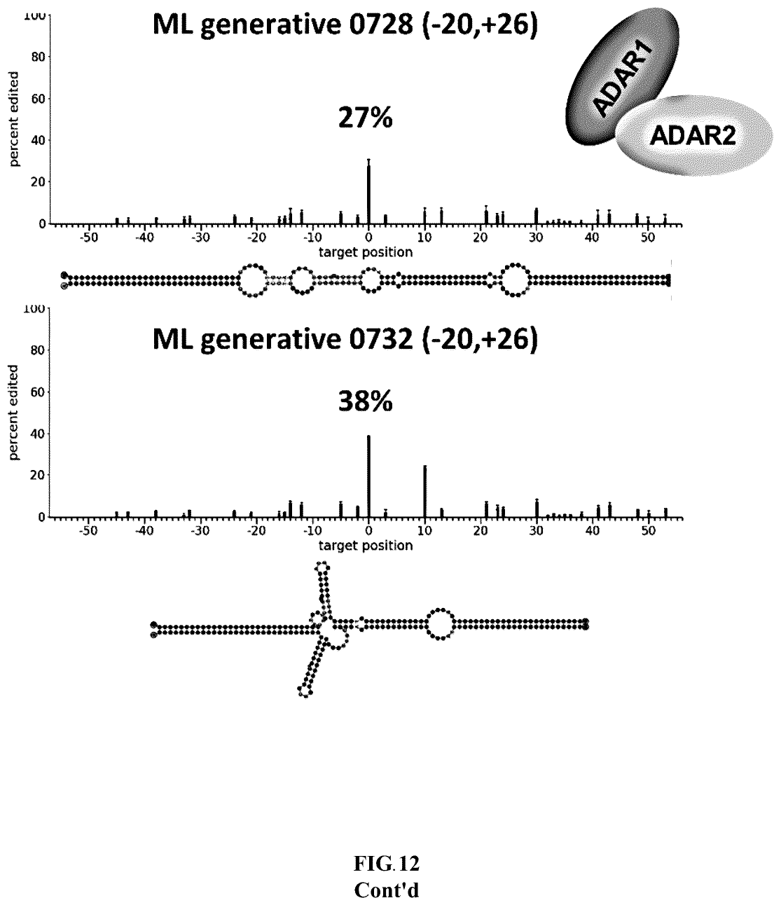

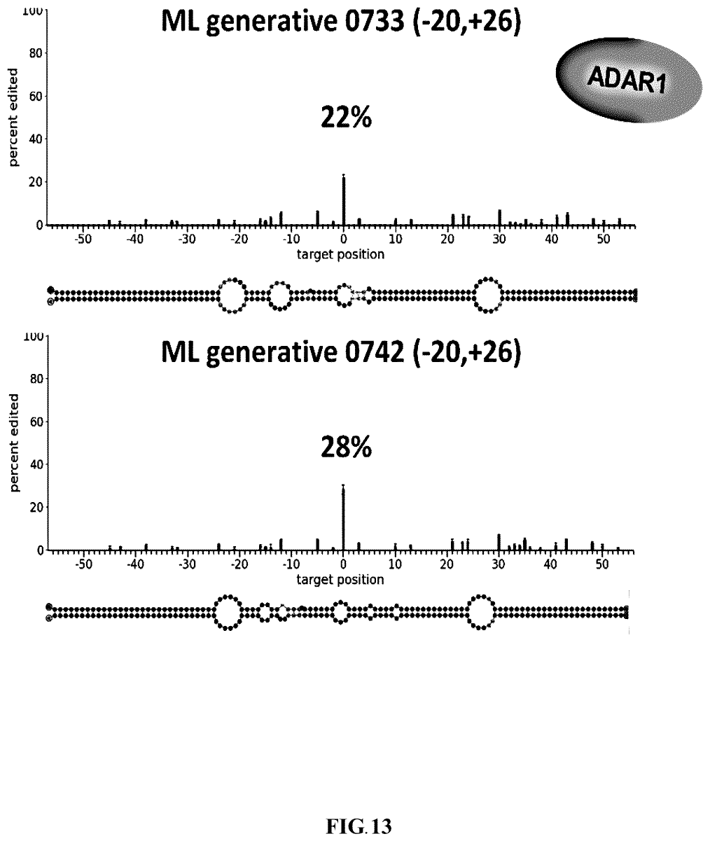

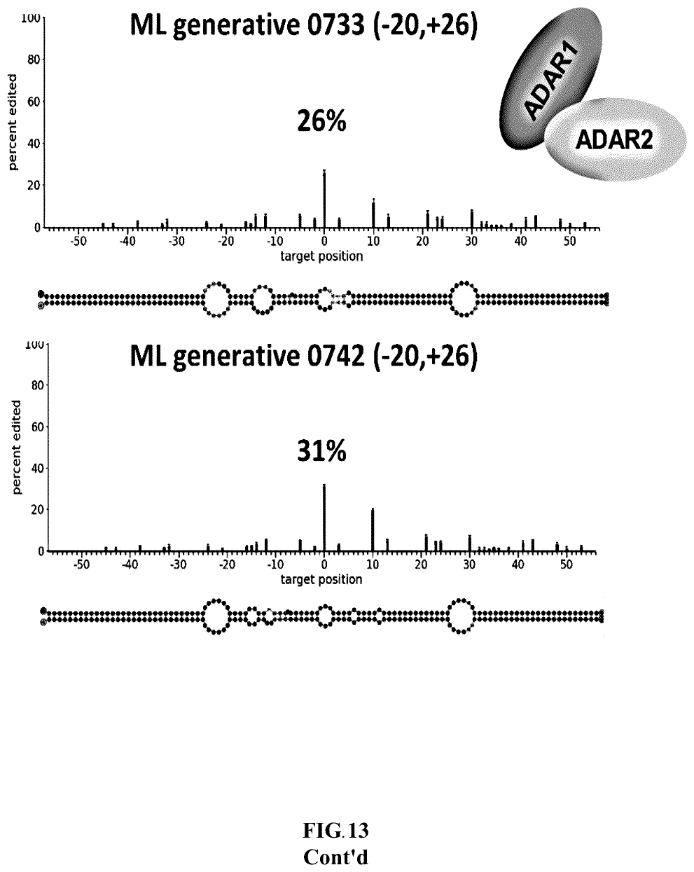

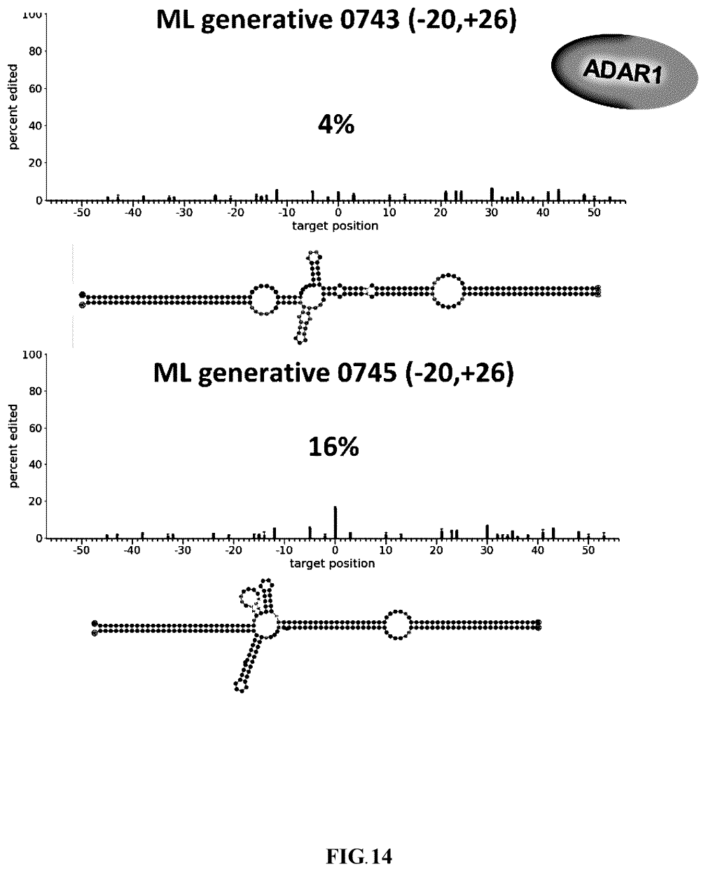

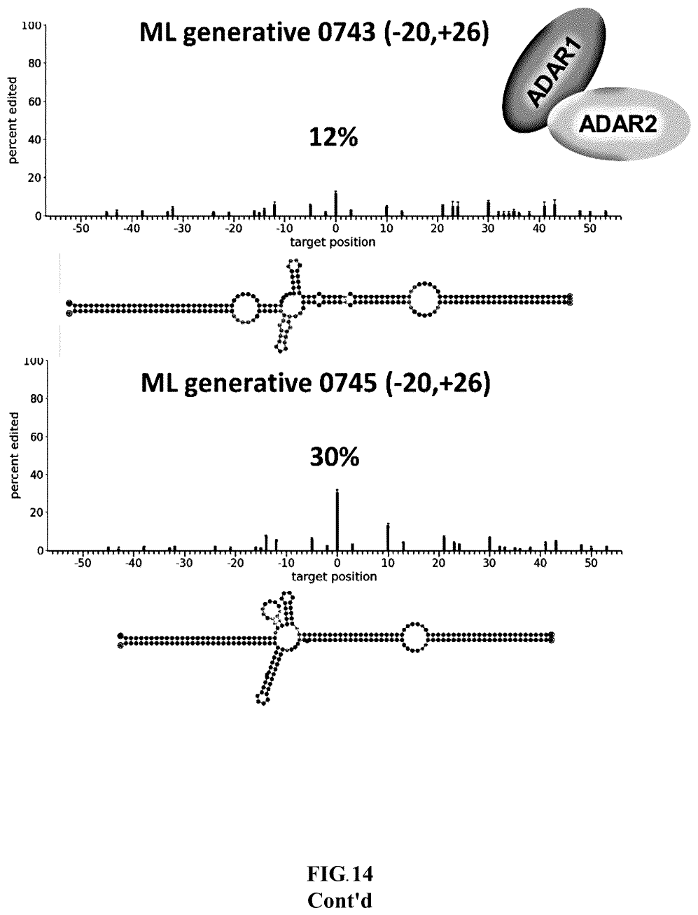

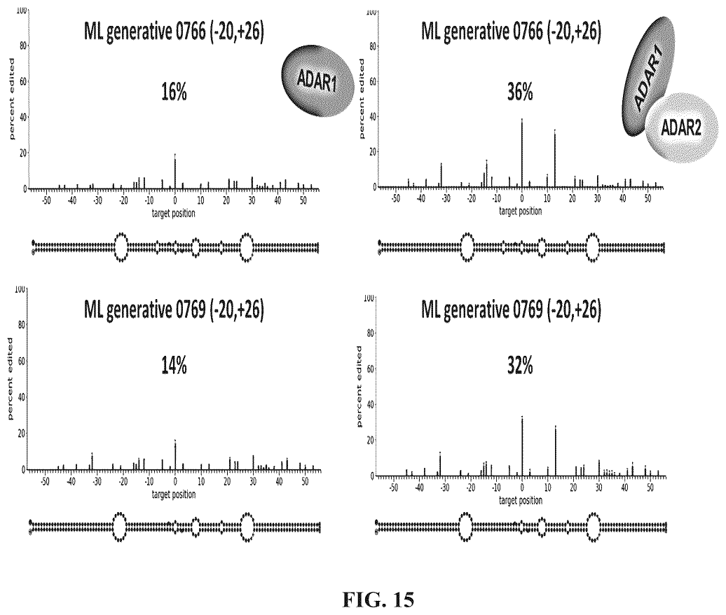

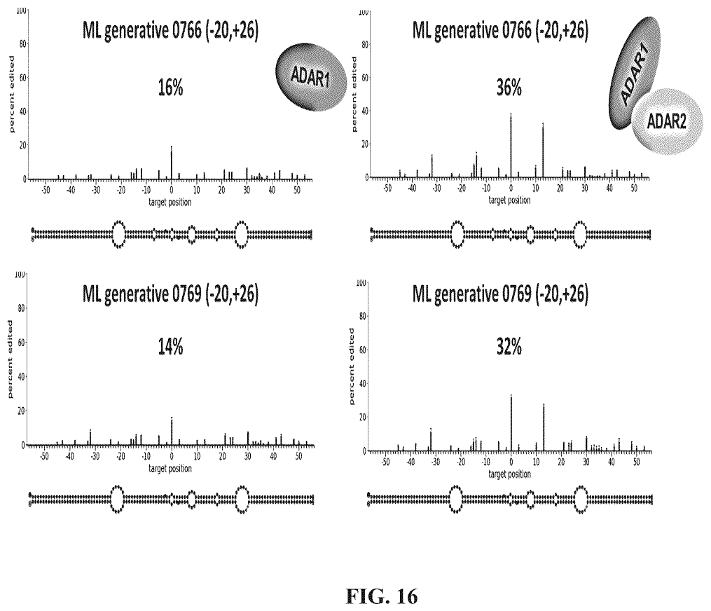

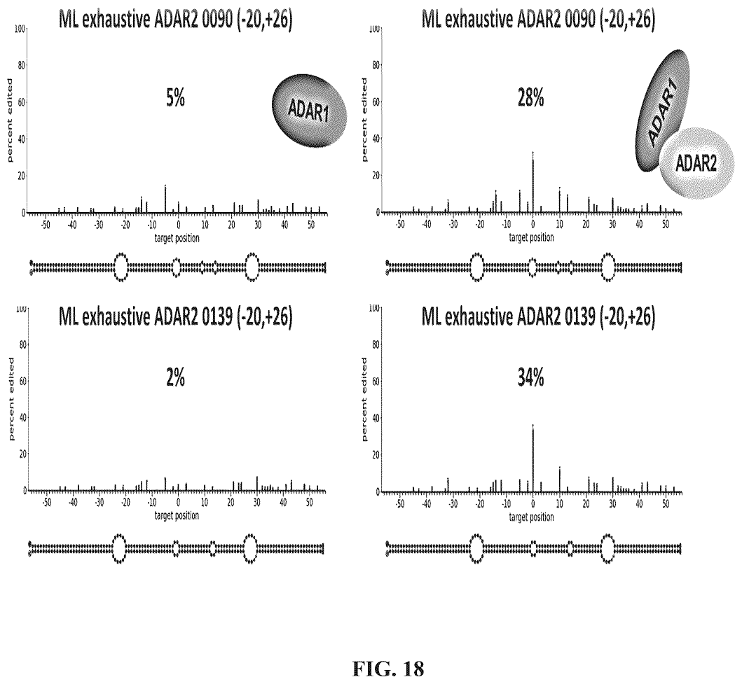

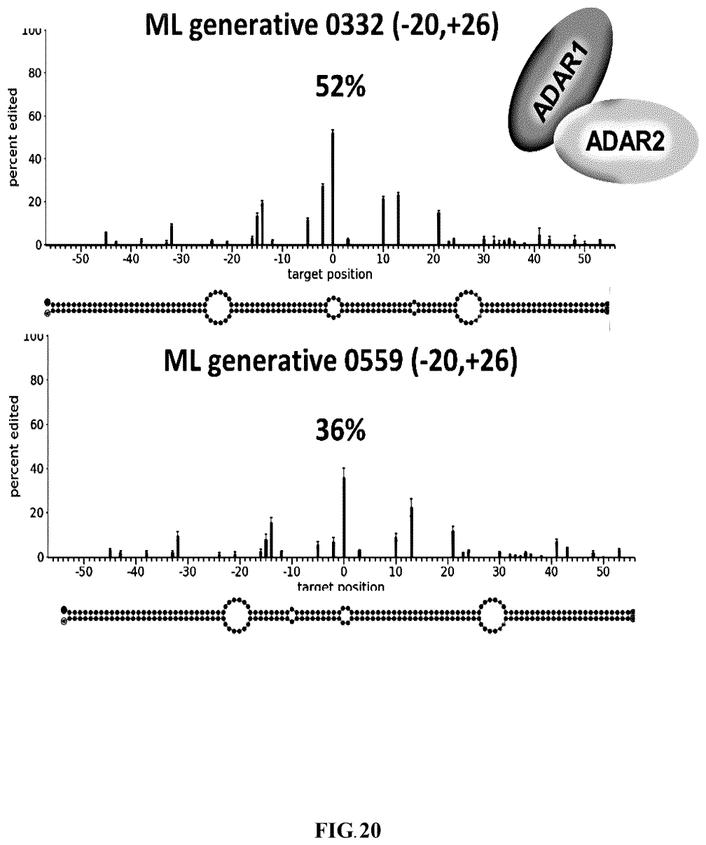

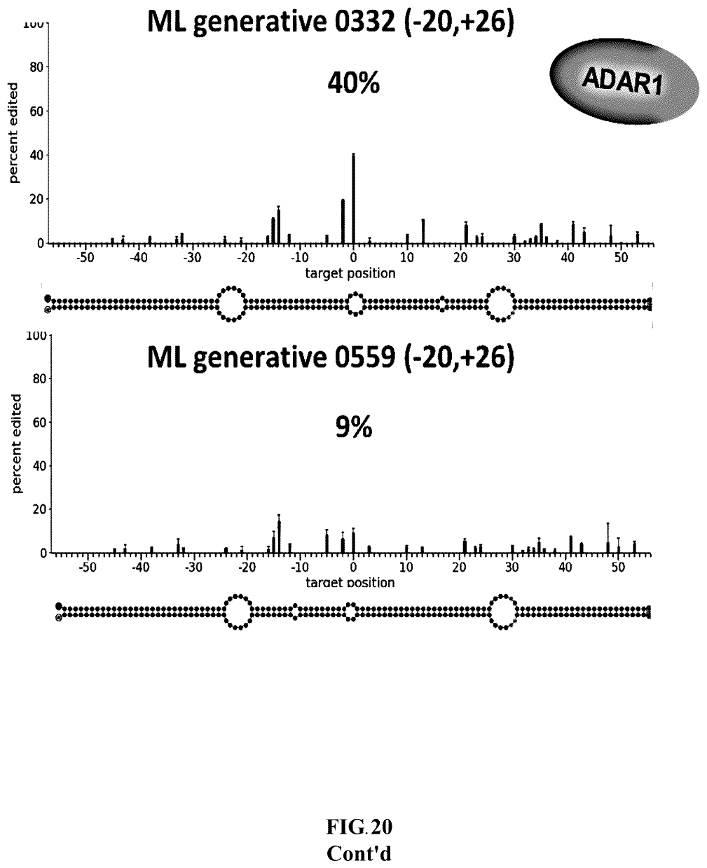

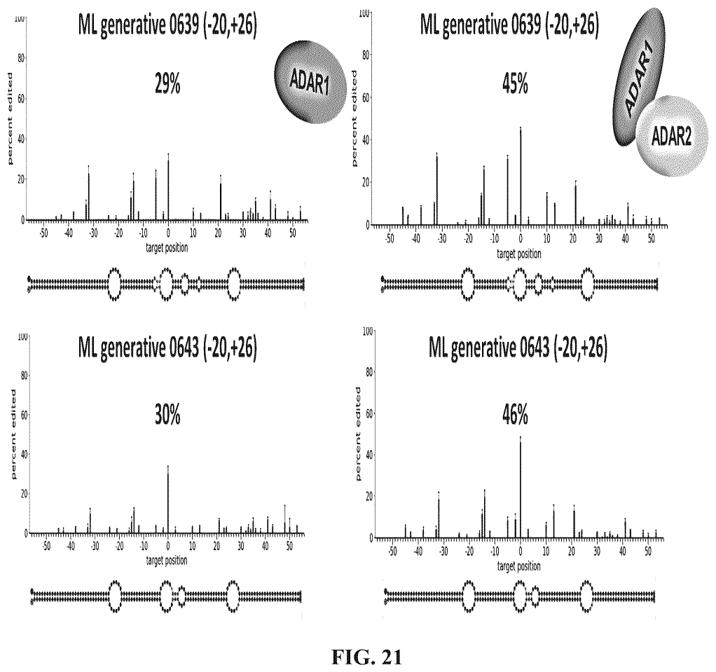

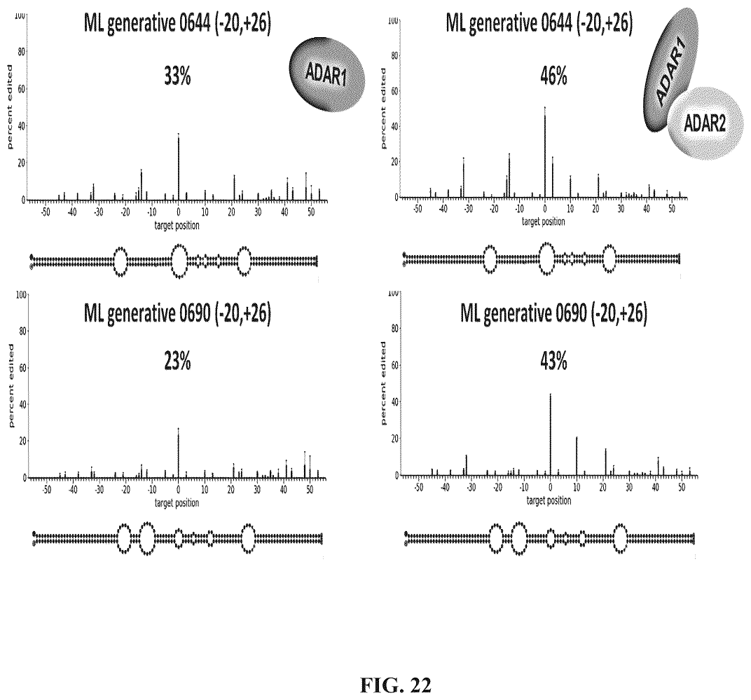

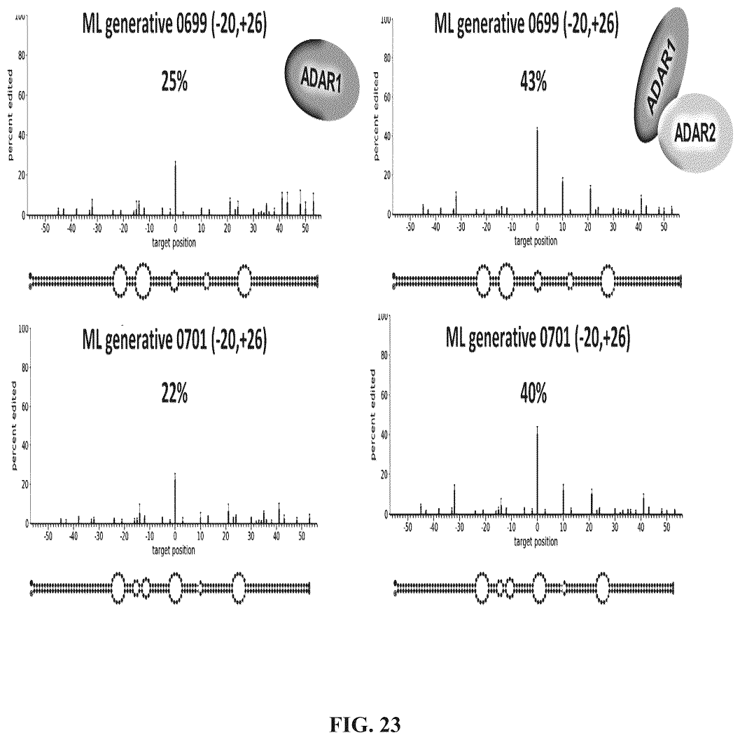

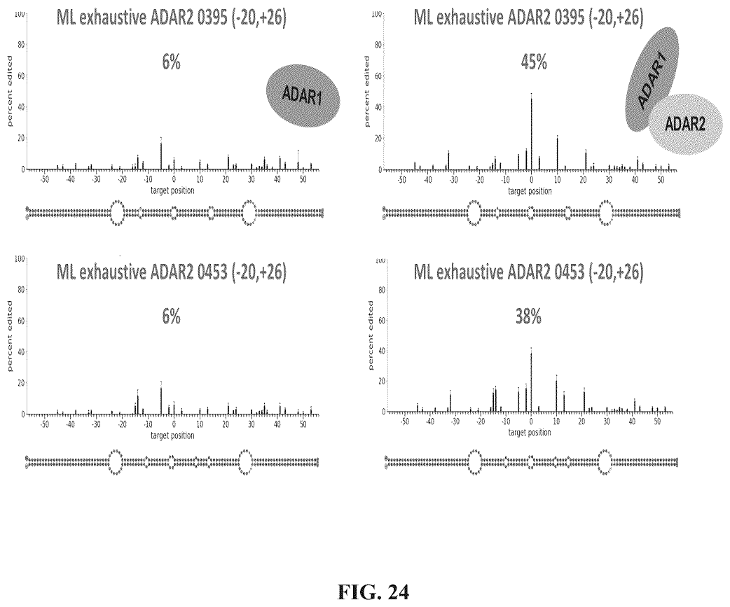

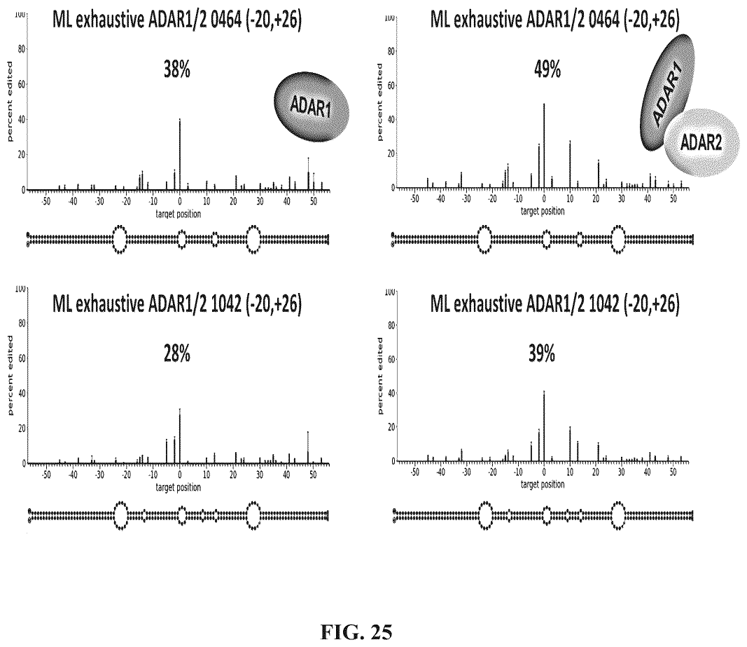

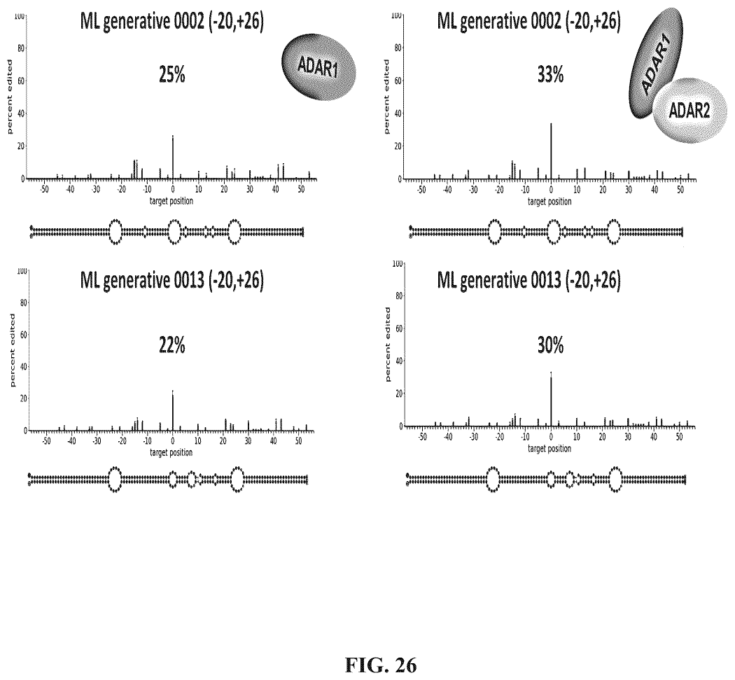

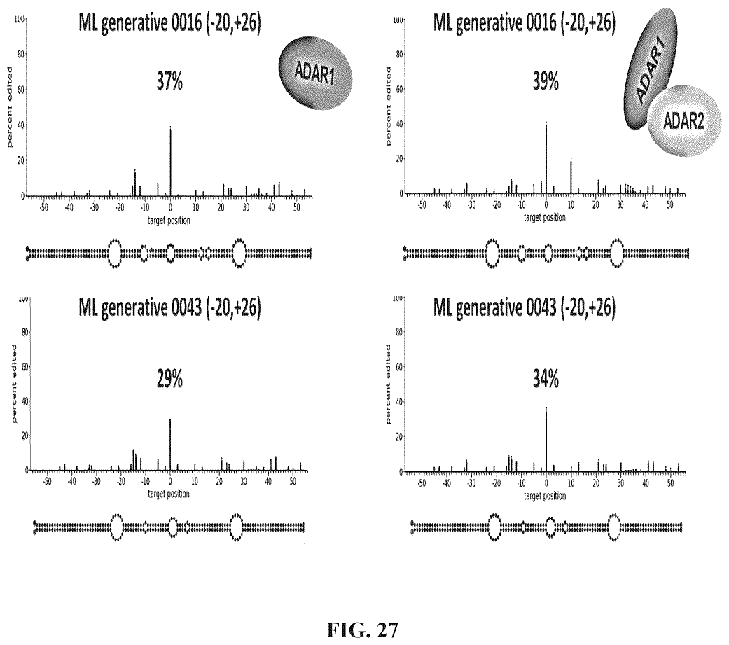

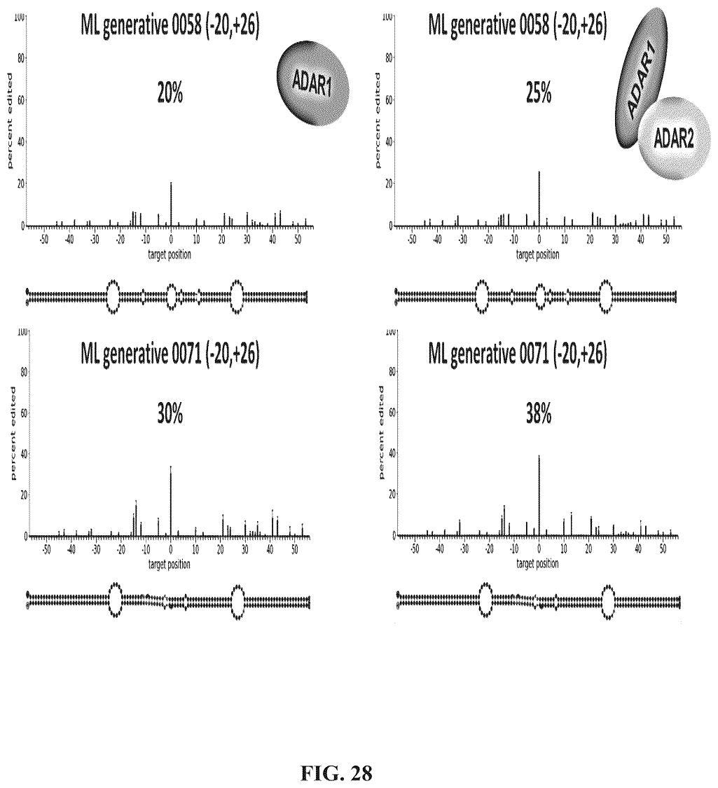

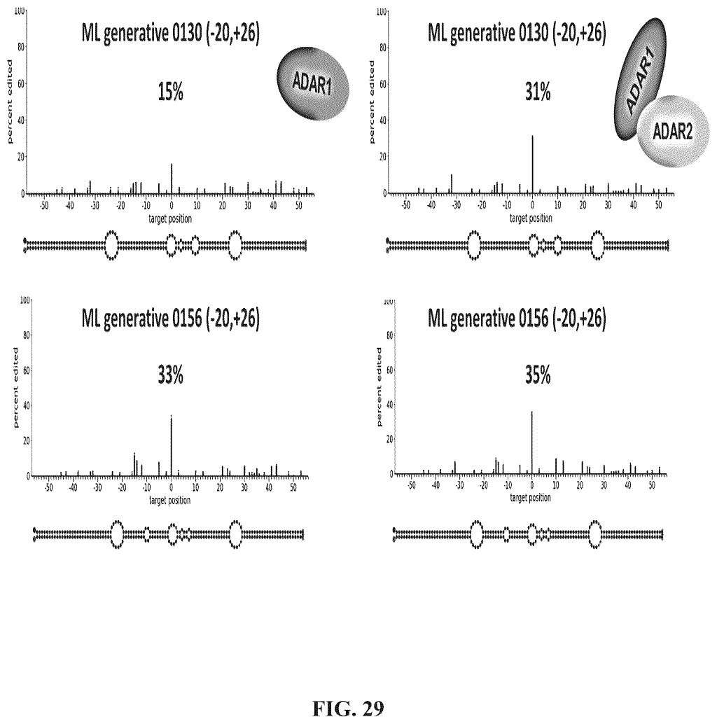

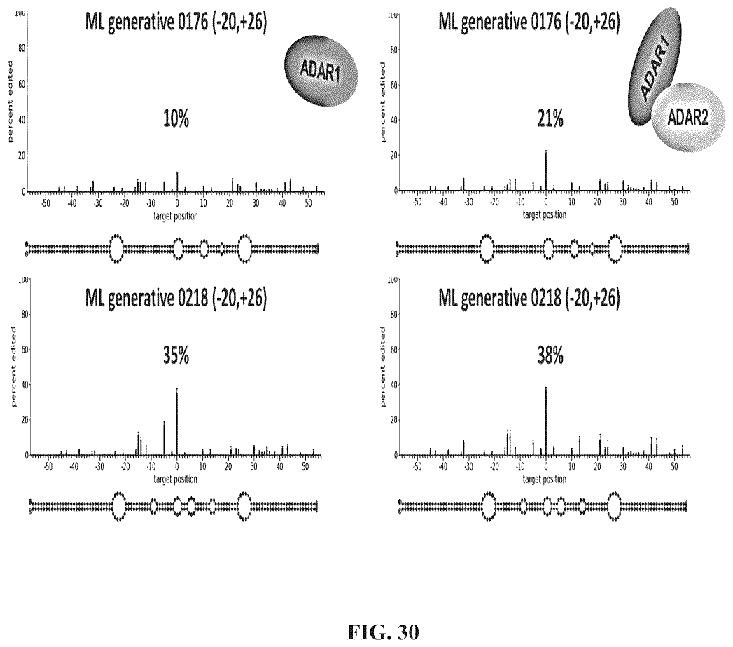

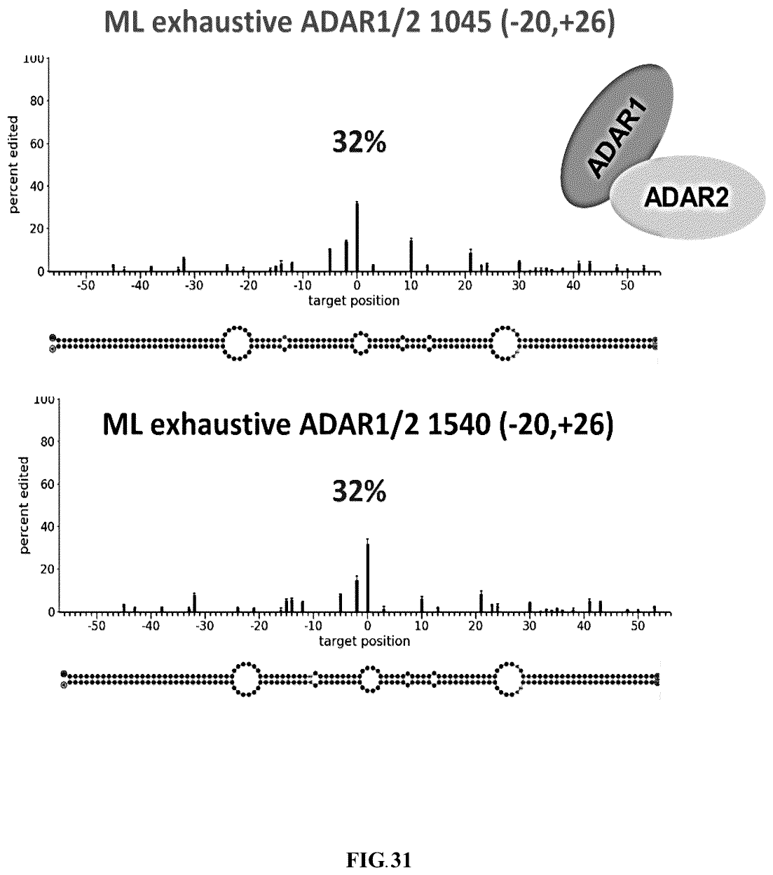

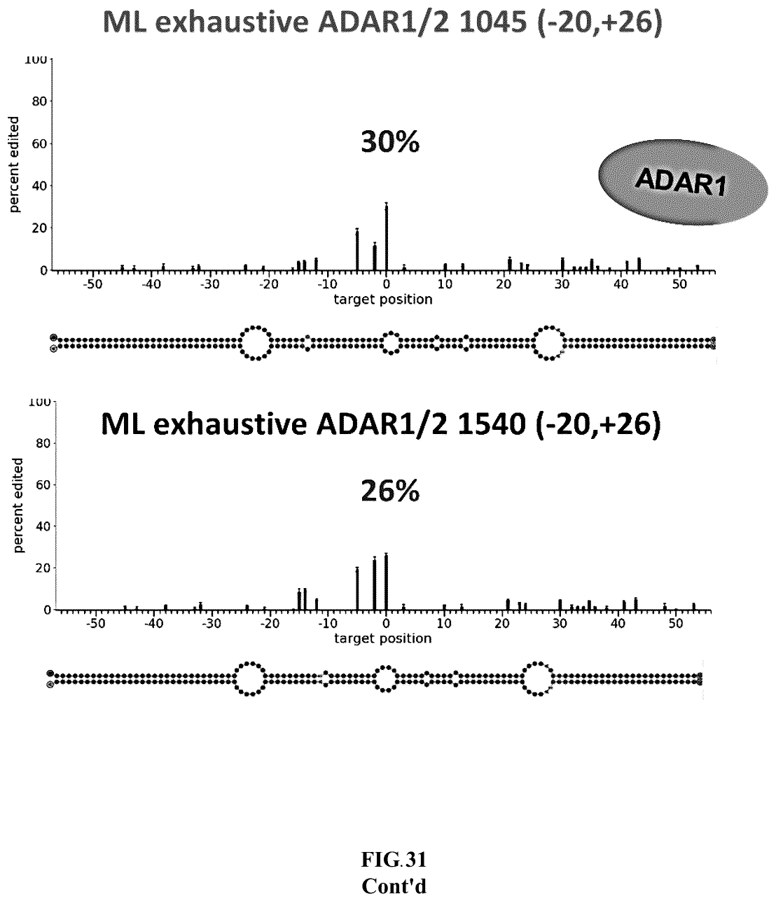

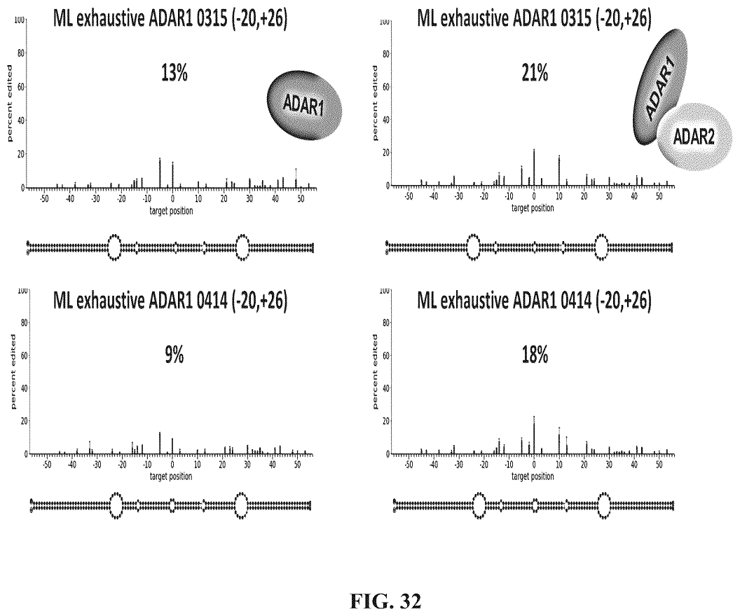

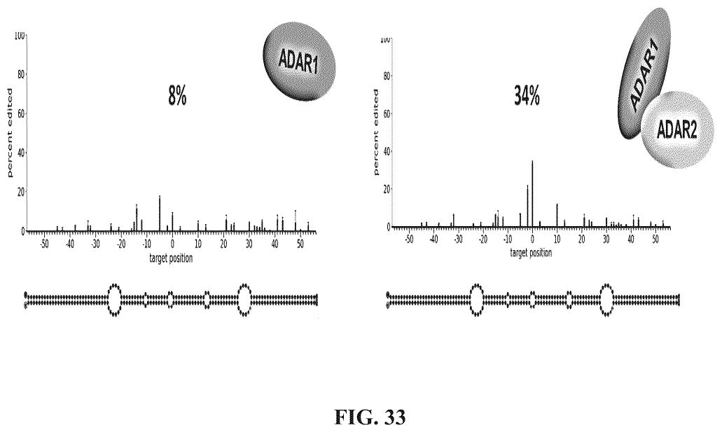

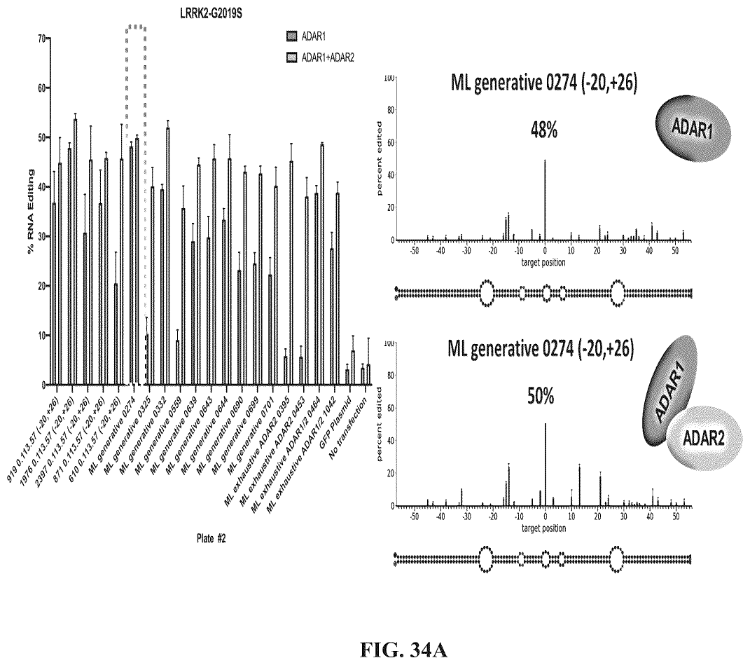

FIG. 8-FIG. 33 show the editing efficiency of each engineered guide RNA via either ADAR1-only or ADAR1+ADAR2. Engineered guide RNAs that facilitated superior editing via ADAR1 and ADAR1+ADAR2 were selected for further engineering. The following LRRK2 engineered guide RNAs recited in TABLE 5 correspond to the editing efficiency plots provided in FIG. 8-FIG. 33. While the engineered guide RNA sequences in TABLE 5 are provided as DNA sequences with a T substituted for each U, the corresponding RNA sequences are also encompassed herein.

TABLE 5
LRRK2 Engineered Guide RNA Sequences Utilized in Example 7

| SEQ | | | | | Left | Right |
|---|---|---|---|---|---|---|
| ID | | | | | Barbell | Barbell |
| NO | FIG. | Guide Name | Sequence | Structural Features (target/guide) | Position | Position |
| 152 | 8 | A-C 0.113.57 (−20, | CCCTGGTGTGCCCTCTGATGTTTTTT | −20_6-6_internal_loop-symmetric_CAUCAU- | −20 | 26 |
| | | +26) | AGGGGATTCTACAGCAGTACTGAGC | UACUAC, 0_1-1_mismatch_A-C, 26_6- | | |
| | | | AATGCCGTAGTCAGCAATCTTTGCA | 6_internal_loop-symmetric_GGGGAU-UAGGGG | | |
| | | | TACTACGCAGCATTGGGATACAGTG | | | |
| | | | TGAAAAGCAGCA | | | |
| 153 | 8 | 919 0.113.57 (−20, | CCCTGGTGTGCCCTCTGATGTTTTTT | −20_6-6_internal_loop-symmetric_CAUCAU- | −20 | 25 |
| | | +26) | AGGGGCTTCTACAGCAGTTCGGAGG | UACUAC, −3_1-1_wobble_U-G, −2_1- | | |
| | | | AATCCCGAGGTCAGCAATCTTTGCA | 1_mismatch_A-A, 0_1-1_mismatch_A-C, 2_1- | | |
| | | | TACTACGCAGCATTGGGATACAGTG | 1_mismatch_C-C, 6_1-1_mismatch_G-G, 10_3- | | |
| | | | TGAAAAGCAGCA | 3_bulge-symmetric_AGU-UCG, 25_7- | | |
| | | | | 7_internal_loop-symmetric_UGGGGAU- | | |
| | | | | UAGGGGC | | |
| 154 | 9 | 1976 0.113.57 (−20, | CCCTGGTGTGCCCTCTGATGTTTTTT | −20_6-6_internal_loop-symmetric_CAUCAU- | −20 | 26 |
| | | +26) | AGGGGATTCTACAACAGTACTGAGC | UACUAC, −8_1-1_mismatch_C-A, −4->5_10- | | |
| | | | TATCCCGAATTCAACAATCTTTGCA | 10_internal_loop-symmetric_CUACAGCAUU- | | |
| | | | TACTACGCAGCATTGGGATACAGTG | UAUCCCGAAU, 17_1-1_mismatch_C-A, 26_6- | | |
| | | | TGAAAAGCAGCA | 6_internal_loop-symmetric_GGGGAU-UAGGGG | | |
| 155 | 9 | 2397 0.113.57 (−20, | CCCTGGTGTGCCCTCTGATGTTTTTT | −49_1-1_wobble_U-G, −20_6-6_internal_loop- | −20 | 26 |
| | | +26) | AGGGGATTCTACCGCTGTGCTGGGC | symmetric_CAUCAU-UACUAC, −3_1- | | |
| | | | AATCCCGTGGTCAGCAATCTTTGCA | 1_wobble_U-G, 0_1-1_mismatch_A-C, 2_1- | | |
| | | | TACTACGCAGCATTGGGATACAGTG | 1_mismatch_C-C, 8_1-1_wobble_U-G, 12_1- | | |
| | | | TGAAAAGCAGCA | 1_wobble_U-G, 15_1-1_mismatch_U-U, 18_1- | | |
| | | | | 1_mismatch_U-C, 26_6-6_internal_loop- | | |
| | | | | symmetric GGGGAU-UAGGGG | | |
| 156 | 10 | 871 0.113.57 (−20, | CCCTGGTGTGCCCTCTGATGTTTTTT | −20_6-6_internal_loop-symmetric_CAUCAU- | −20 | 26 |
| | | +26) | AGGGGATTCTACAGTAGGACTGAGC | UACUAC, −7_1-1_wobble_U-G, −6_1- | | |
| | | | ACTGCCGAGCTGGGCAATCTTTGCA | 1_mismatch_G-G, −4_0-1_bulge-asymmetric_-C, | | |
| | | | TACTACGCAGCATTGGGATACAGTG | −2_1-0_bulge-asymmetric_A-, 0_1-1_mismatch_A- | | |
| | | | TGAAAAGCAGCA | C, 4_1-1_mismatch_U-C, 13_1-1_mismatch_A-G, | | |
| | | | | 16_1-1_wobble_G-U, 26_6-6_internal_loop- | | |
| | | | | symmetric_GGGGAU-UAGGGG | | |
| 157 | 10 | 610 0.113.57 (−20, | CCCTGGTGTGCCCTCTGATGTTTTTT | −20_6-6_internal_loop-symmetric_CAUCAU- | −20 | 26 |
| | | +26) | AGGGGATTCTACATCTGTAGTGAGC | UACUAC, −4_1-1_mismatch_C-C, −3_1- | | |
| | | | AATTCCGTGCTCAGCAATCTTTGCA | 1_wobble_U-G, 0_1-1_mismatch_A-C, 2_1- | | |
| | | | TACTACGCAGCATTGGGATACAGTG | 1_mismatch_C-U, 11_1-1_mismatch_G-G, 15_1- | | |
| | | | TGAAAAGCAGCA | 1_mismatch_U-U, 17_1-1_mismatch_C-U, 26_6- | | |
| | | | | 6_internal_loop-symmetric_GGGGAU-UAGGGG | | |
| 158 | 11 | ML generative 0703 | CCCTGGTGTGCCCTCTGATGTTTTTT | −20_6-6_internal_loop-symmetric_CAUCAU- | −20 | |
| | | (−20, +26) | AGGGGATTCTACGGTAGGACTGATC | UACUAC | | |
| | | | AGTCCCGTGGTCGAACCACGTTGCA | | | |
| | | | TACTACGCAGCATTGGGATACAGTG | | | |
| | | | TGAAAAGCAGCA | | | |
| 159 | 11 | ML generative 0719 | CCCTGGTGTGCCCTCTGATGTTTTTT | −20_6-6_internal_loop-symmetric_CAUCAU- | −20 | 26 |
| | | (−20, +26) | AGGGGATTCTACGGCAAGACTGAGC | UACUAC, −6_9-9_internal_loop- | | |
| | | | AGTCCCGTGGTGGAACCCCGTTGCA | symmetric_AGAUUGCUG-GGAACCCCG, −3_1- | | |
| | | | TACTACGCAGCATTGGGATACAGTG | 1_wobble_U-G, 0_1-1_mismatch_A-C, 2_1- | | |
| | | | TGAAAAGCAGCA | 1_mismatch_C-C, 4_1-1_wobble_U-G, 13_2- | | |
| | | | | 2_bulge-symmetric_AC-AG, 18_1-1_wobble_U-G, | | |
| | | | | 26_6-6_internal_loop-symmetric_GGGGAU- | | |
| | | | | UAGGGG | | |
| 160 | 12 | ML generative 0728 | CCCTGGTGTGCCCTCTGATGTTTTTT | −20_6-6_internal_loop-symmetric_CAUCAU- | −20 | 26 |
| | | (−20, +26) | AGGGGATCCTACGGCAGTACTGATC | UACUAC, −11_4-5_internal_loop- | | |
| | | | AAATCCGTGGTGGCACCCCGTTGCA | asymmetric_AGAU-CCCCG, −7_1-1_wobble_U-G, | | |
| | | | TACTACGCAGCATTGGGATACAGTG | −6_1-0_bulge-asymmetric_G-, −3_1-1_wobble_U- | | |
| | | | TGAAAAGCAGCA | G, 0_1-1_mismatch_A-C, 2_2−2_bulge- | | |
| | | | | symmetric_CA-AU, 7_1-1_mismatch_C-U, 18_1- | | |
| | | | | 1_wobble_U-G, 23_1-1_mismatch_A-C, 26_6- | | |
| | | | | 6_internal_loop-symmetric_GGGGAU-UAGGGG | | |
| 161 | 12 | ML generative 0732 | CCCTGGTGTGCCCTCTGATGTTTTTT | −20_6-6_internal_loop-symmetric_CAUCAU- | −20 | 26 |
| | | (−20, +26) | AGGGGATTCTACAGCAGGACTGACC | UACUAC, −14_2−2_bulge-symmetric_AA-CG, | | |
| | | | AGTTCCGTGGTGGCACCTCCGTGCA | −11_1−2_bulge-asymmetric_U-CC, −7_1- | | |
| | | | TACTACGCAGCATTGGGATACAGTG | 1_wobble_U-G, −6_1-0_bulge-asymmetric_G-, | | |
| | | | TGAAAAGCAGCA | −3_1-1_wobble_U-G, 0_1-1_mismatch_A-C, 2_1- | | |
| | | | | 1_mismatch_C-U, 4_1-1_wobble_U-G, 7_1- | | |
| | | | | 1_mismatch_C-C, 13_1-1_mismatch_A-G, 26_6- | | |
| | | | | 6_internal_loop-symmetric_GGGGAU-UAGGGG | | |
| 162 | 13 | ML_generative_0733 | CCCTGGTGTGCCCTCTGATGTTTTTT | −20_6-6_internal_loop-symmetric_CAUCAU- | −20 | 26 |
| | | (−20, +26) | AGGGGATTCTACGGCAGTACTGAGA | UACUAC, −11_4-5_internal_loop- | | |
| | | | AATCCCGTGGTGGCACCCCGTTGCA | asymmetric_AGAU-CCCCG, −7_1-1_wobble_U-G, | | |
| | | | TACTACGCAGCATTGGGATACAGTG | −6_1-0_bulge-asymmetric_G-, −3_1-1_wobble_U- | | |
| | | | TGAAAAGCAGCA | G, 0_1-1_mismatch_A-C, 2_1-1_mismatch_C-C, | | |
| | | | | 6_1-1_mismatch_G-A, 18_1-1_wobble_U-G, 26_6- | | |
| | | | | 6_internal_loop-symmetric GGGGAU-UAGGGG | | |
| 163 | 13 | ML_generative_0742 | CCCTGGTGTGCCCTCTGATGTTTTTT | −20_6-6_internal_loop-symmetric_CAUCAU- | −20 | 26 |
| | | (−20, +26) | AGGGGATTCTACAGCAGTCCTGACC | UACUAC, −14_2−2_bulge-symmetric_AA-CG, | | |
| | | | AGTCCCGTGGTCGTACCTCCGTGCA | −11_1−2_bulge-asymmetric_U-CC, −9_1- | | |
| | | | TACTACGCAGCATTGGGATACAGTG | 1_wobble_G-U, −7_1-0_bulge-asymmetric_U-, | | |
| | | | TGAAAAGCAGCA | −3_1-1_wobble_U-G, 0_1-1_mismatch_A-C, 2_1- | | |
| | | | | 1_mismatch_C-C, 4_1-1_wobble_U-G, 7_1- | | |
| | | | | 1_mismatch_C-C, 12_1-1_mismatch_U-C, 26_6- | | |
| | | | | 6 internal_loop-symmetric_GGGGAU-UAGGGG | | |
| 164 | 14 | ML_generative_0743 | CCCTGGTGTGCCCTCTGATGTTTTTT | −20_6-6_internal_loop-symmetric_CAUCAU- | −20 | |
| | | (−20, +26) | AGGGGCTTCTACAGCAGGACTGATC | UACUAC | | |
| | | | AAATCCGTGGTGGAACCCCGTTGCA | | | |
| | | | TACTACGCAGCATTGGGATACAGTG | | | |
| | | | TGAAAAGCAGCA | | | |
| 165 | 14 | ML_generative_0745 | CCCTGGTGTGCCCTCTGATGTTTTTT | | | |
| | | (−20, +26) | AGGGGATTCTACGGCAGTACTGAGA | | | |
| | | | CATCCCGTGGTGGCACCCCCGTGCA | | | |
| | | | TACTACGCAGCATTGGGATACAGTG | | | |
| | | | TGAAAAGCAGCA | | | |
| 166 | 15 | ML_generative_0766 | CCCTGGTGTGCCCTCTGATGTTTTTT | −20_6-6_internal_loop-symmetric_CAUCAU- | −20 | 26 |
| | 16 | (−20, +26) | AGGGGATTCTACCGCAGTACTCACC | UACUAC, −8_1-1_mismatch_C-U, −7_1- | | |
| | | | AGTCGCCGTGTCGTCAATCTTTGCA | 1_wobble_U-G, −3_1-0_bulge-asymmetric_U-, | | |
| | | | TACTACGCAGCATTGGGATACAGTG | 0_1-1_mismatch_A-C, 2_0-1_bulge-asymmetric_- | | |
| | | | TGAAAAGCAGCA | C, 4_1-1_wobble_U-G, 7_3−3_bulge- | | |
| | | | | symmetric_CUC-CAC, 18_1-1_mismatch_U-C, | | |
| | | | | 26_6-6_internal_loop-symmetric_GGGGAU- | | |
| | | | | UAGGGG | | |
| 167 | 15 | ML_generative_0769 | CCCTGGTGTGCCCTCTGATGTTTTTT | −20_6-6_internal_loop-symmetric_CAUCAU- | −20 | 26 |
| | 16 | (−20, +26) | AGGGGATTCTACCGCAGTACTCACC | UACUAC, −7_1-1_wobble_U-G, −3_1-0_bulge- | | |
| | | | AGTCGCCGTGTCGGCAATCTTTGCA | asymmetric_U-, 0_1-1_mismatch_A-C, 2_0- | | |
| | | | TACTACGCAGCATTGGGATACAGTG | 1_bulge-asymmetric_-C, 4_1-1_wobble_U-G, 7_3- | | |
| | | | TGAAAAGCAGCA | 3_bulge-symmetric_CUC-CAC, 18_1- | | |
| | | | | 1_mismatch_U-C, 26_6-6_internal_loop- | | |
| | | | | symmetric GGGGAU-UAGGGG | | |
| 168 | 17 | ML_exhaustive_ADAR2_ | CCCTGGTGTGCCCTCTGATGTTTTTT | −20_6-6_internal_loop-symmetric_CAUCAU- | −20 | 26 |
| | 36 | 0049 (−20, +26) | AGGGGATTCTACAGCATTACTCAGC | UACUAC, −11_1-1_mismatch_U-C, −1->0_2- | | |
| | | | AGTGCGTTAGTCAGCACTCTTTGCA | 2_bulge-symmetric_CA-GU, 4_1-1_wobble_U-G, | | |
| | | | TACTACGCAGCATTGGGATACAGTG | 9_1-1_mismatch_C-C, 14_1-1_mismatch_C-U, | | |
| | | | TGAAAAGCAGCA | 26_6-6_internal_loop-symmetric_GGGGAU- | | |
| | | | | UAGGGG | | |
| 169 | 17 | ML_exhaustive_ADAR2_ | CCCTGGTGTGCCCTCTGATGTTTTTT | −20_6-6_internal_loop-symmetric_CAUCAU- | −20 | 26 |
| | 36 | 0069 (−20, +26) | AGGGGATTCTACAGCACGACTGAGC | UACUAC, −7_1-1_wobble_U-G, −1->0_2−2_bulge- | | |
| | | | AGTGCGTTAGTCGGCAATCTTTGCA | symmetric_CA-GU, 4_1-1_wobble_U-G, 13_2- | | |
| | | | TACTACGCAGCATTGGGATACAGTG | 2_bulge-symmetric_AC-CG, 26_6-6_internal_loop- | | |
| | | | TGAAAAGCAGCA | symmetric_GGGGAU-UAGGGG | | |
| 170 | 18 | ML_exhaustive_ADAR2_ | CCCTGGTGTGCCCTCTGATGTTTTTT | −20_6-6_internal_loop-symmetric_CAUCAU- | −20 | 26 |
| | 36 | 0090 (−20, +26) | AGGGGATTCTACAGCAATACTCAGC | UACUAC, −2->0_3−3_bulge-symmetric_ACA- | | |
| | | | AGTGCGTGAGTCAGCAATCTTTGCA | GUG, 4_1-1_wobble_U-G, 9_1-1_mismatch_C-C, | | |
| | | | TACTACGCAGCATTGGGATACAGTG | 14_1-1_mismatch_C-A, 26_6-6_internal_loop- | | |
| | | | TGAAAAGCAGCA | symmetric GGGGAU-UAGGGG | | |
| 171 | 18 | ML exhaustive ADAR2_ | CCCTGGTGTGCCCTCTGATGTTTTTT | −20_6-6_internal_loop-symmetric_CAUCAU- | −20 | 26 |
| | 36 | 0139 (−20, +26) | AGGGGATTCTACAGCACGACTGAGC | UACUAC, −7_1-1_wobble_U-G, −1->0_2−2_bulge- | | |
| | | | AGTGCGCTAGTCGGCAATCTTTGCA | symmetric_CA-GC, 4_1-1_wobble_U-G, 13 2- | | |
| | | | TACTACGCAGCATTGGGATACAGTG | 2_bulge-symmetric_AC-CG, 26_6-6_internal_loop- | | |
| | | | TGAAAAGCAGCA | symmetric_GGGGAU-UAGGGG | | |
| 172 | 19 | ML_generative_0274 | CCCTGGTGTGCCCTCTGATGTTTTTT | −20_6-6_internal_loop-symmetric_CAUCAU- | −20 | 26 |
| | | (−20, +26) | AGGGGATTCTACAGCAGTACTGTCC | UACUAC, −8_2−2_bulge-symmetric_GC-UA, | | |
| | | | AGTCCCGTGGTCGTAAATCTTTGCA | −7_1-1_wobble_U-G, −3_1-1_wobble_U-G, 0_1- | | |
| | | | TACTACGCAGCATTGGGATACAGTG | 1_mismatch_A-C, 2_1-1_mismatch_C-C, 4_1- | | |
| | | | TGAAAAGCAGCA | 1_wobble_U-G, 7_2−2_bulge-symmetric_CU-UC, | | |
| | | | | 26_6-6_internal_loop-symmetric_GGGGAU- | | |
| | | | | UAGGGG | | |
| 173 | 19 | ML_generative_0325 | CCCTGGTGTGCCCTCTGATGTTTTTT | −20 6-6_internal_loop-symmetric_CAUCAU- | −20 | 26 |
| | | (−20, +26) | AGGGGATTCTACATCAGGACTGAGC | UACUAC, −10_1-1_mismatch_U-U, −7_1- | | |
| | | | TTTTCCGAGGTCGGCTATCTTTGCAT | 1_wobble_U-G, −3_1-1_wobble_U-G, −2->5_8- | | |
| | | | ACTACGCAGCATTGGGATACAGTGT | 8_internal_loop-symmetric_ACAGCAUU- | | |
| | | | GAAAAGCAGCA | UUUUCCGA, 13_1-1_mismatch_A-G, 17_1- | | |
| | | | | 1_mismatch_C-U, 26_6-6_internal_loop- | | |
| | | | | symmetric_GGGGAU-UAGGGG | | |
| 174 | 20 | ML_generative_0332 | CCCTGGTGTGCCCTCTGATGTTTTTT | −20 6-6_internal_loop-symmetric_CAUCAU- | −20 | 26 |
| | | (−20, +26) | AGGGGATTCTACATCAGTACTGGGC | UACUAC, −7_1-1_wobble_U-G, 0_1- | | |
| | | | AGTTCCGTAGTCGGCAATCTTTGCA | 1_mismatch_A-C, 2_1-1_mismatch_C-U, 4_1- | | |
| | | | TACTACGCAGCATTGGGATACAGTG | 1_wobble_U-G, 8_1-1_wobble_U-G, 17_1- | | |
| | | | TGAAAAGCAGCA | 1_mismatch_C-U, 26_6-6_internal_loop- | | |
| | | | | symmetric_GGGGAU-UAGGGG | | |
| 175 | 20 | ML_generative_0559 | CCCTGGTGTGCCCTCTGATGTTTTTT | −20 6-6_internal_loop-symmetric_CAUCAU- | −20 | 26 |
| | | (−20, +26) | AGGGGATTCTACAGCAGTACTGGGC | UACUAC, −11_1-1_mismatch_U-C, −7_1- | | |
| | | | AGTGCGCTAGTCGGCACTCTTTGCA | 1_wobble_U-G, −1->0_2−2_bulge-symmetric_CA- | | |
| | | | TACTACGCAGCATTGGGATACAGTG | GC, 4_1-1_wobble_U-G, 8_1-1_wobble_U-G, | | |
| | | | TGAAAAGCAGCA | 26_6-6_internal_loop-symmetric_GGGGAU- | | |
| | | | | UAGGGG | | |
| 176 | 21 | ML_generative_0639 | CCCTGGTGTGCCCTCTGATGTTTTTT | −20_6-6_internal_loop-symmetric_CAUCAU- | −20 | 26 |
| | | (−20, +26) | AGGGGATTCTACAGCAATACTCACC | UACUAC, −6_1-1_mismatch_G-A, −3->2_6- | | |
| | | | AGTCCCGATGTAAGCAATCTTTGCA | 6_internal_loop-symmetric_UACAGC-CCCGAU, | | |
| | | | TACTACGCAGCATTGGGATACAGTG | 4_1-1_wobble_U-G, 7_3−3_bulge- | | |
| | | | TGAAAAGCAGCA | symmetric_CUC-CAC, 14_1-1_mismatch_C-A, | | |
| | | | | 26_6-6_internal_loop-symmetric_GGGGAU- | | |
| | | | | UAGGGG | | |
| 177 | 21 | ML_generative_0643 | CCCTGGTGTGCCCTCTGATGTTTTTT | −20 6-6_internal_loop-symmetric_CAUCAU- | −20 | 26 |
| | | (−20, +26) | AGGGGATTCTACGGCAGTACTGTGA | UACUAC, −7_1-1_wobble_U-G, −3->6_10- | | |
| | | | AATCCCGATGTCGGCAATCTTTGCA | 10_internal_loop-symmetric_UACAGCAUUG- | | |
| | | | TACTACGCAGCATTGGGATACAGTG | AAAUCCCGAU, 8_1-1_mismatch_U-U, 18_1- | | |
| | | | TGAAAAGCAGCA | 1_wobble_U-G, 26_6-6_internal_loop- | | |
| | | | | symmetric_GGGGAU-UAGGGG | | |
| 178 | 22 | ML_generative_0644 | CCCTGGTGTGCCCTCTGATGTTTTTT | −20 6-6_internal_loop-symmetric_CAUCAU- | −20 | 26 |
| | | (−20, +26) | AGGGGATTCTACCGCAGGACGGAG | UACUAC, −7_0-1_bulge-asymmetric _-G, −6_1- | | |
| | | | CTATCCCGAGTTAGGCAATCTTTGC | 1_wobble_G-U, −2->5_8-7_internal_loop- | | |
| | | | ATACTACGCAGCATTGGGATACAGT | asymmetric_ACAGCAUU-UAUCCCG, 10_1- | | |
| | | | GTGAAAAGCAGCA | 1_mismatch_A-G, 13_1-1_mismatch_A-G, 18_1- | | |
| | | | | 1_mismatch_U-C, 26_6-6_internal_loop- | | |
| | | | | symmetric_GGGGAU-UAGGGG | | |
| 179 | 22 | ML_generative_0690 | CCCTGGTGTGCCCTCTGATGTTTTTT | −20_6-6_internal_loop-symmetric_CAUCAU- | −20 | 26 |
| | | (−20, +26) | AGGGGATTCTACGGCATGACTGATC | UACUAC, −14_2−2_bulge-symmetric_AA-GA, | | |
| | | | AGTCCCGTGGTCGGACCCCGATGCA | −9_4−4_bulge-symmetric_AUUG-ACCC, −7_1- | | |
| | | | TACTACGCAGCATTGGGATACAGTG | 1_wobble_U-G, −3_1-1_wobble_U-G, 0_1- | | |
| | | | TGAAAAGCAGCA | 1_mismatch_A-C, 2_1-1_mismatch_C-C, 4_1- | | |
| | | | | 1_wobble_U-G, 7_1-1_mismatch_C-U, 13_2- | | |
| | | | | 2_bulge-symmetric_AC-UG, 18_1-1_wobble_U-G, | | |
| | | | | 26_6-6_internal_loop-symmetric_GGGGAU- | | |
| | | | | UAGGGG | | |
| 180 | 23 | ML_generative_0699 | CCCTGGTGTGCCCTCTGATGTTTTTT | −20_6-6_internal_loop-symmetric_CAUCAU- | −20 | 26 |
| | | (−20, +26) | AGGGGATTCTACGGCAAGACTGGGC | UACUAC, −9_7-7_internal_loop- | | |
| | | | AGTCCCGTGGTCGGACCCCCGTGCA | symmetric_AAGAUUG-ACCCCCG, −7_1- | | |
| | | | TACTACGCAGCATTGGGATACAGTG | 1_wobble_U-G, −3_1-1_wobble_U-G, 0_1- | | |
| | | | TGAAAAGCAGCA | 1_mismatch_A-C, 2_1-1_mismatch_C-C, 4_1- | | |
| | | | | 1_wobble_U-G, 8_1-1_wobble_U-G, 13 2- | | |
| | | | | 2_bulge-symmetric_AC-AG, 18_1-1_wobble_U-G, | | |
| | | | | 26_6-6_internal_loop-symmetric_GGGGAU- | | |
| | | | | UAGGGG | | |
| 181 | 23 | ML_generative_0701 | CCCTGGTGTGCCCTCTGATGTTTTTT | −20_6-6_internal_loop-symmetric_CAUCAU- | −20 | 26 |
| | | (−20, +26) | AGGGGATTCTACGGCAGGACTGGGC | UACUAC, −14_2−2_bulge-symmetric_AA-CG, | | |
| | | | TTTCCCGTGGTCGGACCTCCGTGCA | −9_3−3_bulge-symmetric_UUG-ACC, −7_1- | | |
| | | | TACTACGCAGCATTGGGATACAGTG | 1_wobble_U-G, −3_1-1_wobble_U-G, 0 1- | | |
| | | | TGAAAAGCAGCA | 1_mismatch_A-C, 2_1-1_mismatch_C-C, 4_2- | | |
| | | | | 2_bulge-symmetric_UU-UU, 8_1-1_wobble_U-G, | | |
| | | | | 13_1-1_mismatch_A-G, 18_1-1_wobble_U-G, | | |
| | | | | 26_6-6_internal_loop-symmetric_GGGGAU- | | |
| | | | | UAGGGG | | |
| 182 | 24 | ML exhaustive_ADAR2_ | CCCTGGTGTGCCCTCTGATGTTTTTT | −20_6-6_internal_loop-symmetric_CAUCAU- | −20 | 26 |
| | 36 | 0395 (−20, +26) | AGGGGATTCTACAGCACGACTGAGC | UACUAC, −13_1-1_mismatch_G-A, −1->0 2- | | |
| | | | AGTGCGCTAGTCAGCAATATTTGCA | 2_bulge-symmetric_CA-GC, 4_1-1_wobble_U-G, | | |
| | | | TACTACGCAGCATTGGGATACAGTG | 13_2−2_bulge-symmetric_AC-CG, 26 6- | | |
| | | | TGAAAAGCAGCA | 6_internal_loop-symmetric_GGGGAU-UAGGGG | | |
| 183 | 24 | ML_exhaustive_ADAR2_ | CCCTGGTGTGCCCTCTGATGTTTTTT | −20_6-6_internal_loop-symmetric_CAUCAU- | −20 | 26 |
| | 36 | 0453 (−20, +26) | AGGGGATTCTACAGCAATACTCAGC | UACUAC, −10_1-1_mismatch_U-C, −1->0_2- | | |
| | | | AGTGCGCTAGTCAGCCATCTTTGCA | 2_bulge-symmetric_CA-GC, 4_1-1_wobble_U-G, | | |
| | | | TACTACGCAGCATTGGGATACAGTG | 9_1-1_mismatch_C-C, 14_1-1_mismatch_C-A, | | |
| | | | TGAAAAGCAGCA | 26_6-6_internal_loop-symmetric_GGGGAU- | | |
| | | | | UAGGGG | | |
| 184 | 25 | ML_exhaustive_ADAR1/2_ | CCCTGGTGTGCCCTCTGATGTTTTTT | −20_6-6_internal_loop-symmetric_CAUCAU- | −20 | 26 |
| | | 0464 (−20, +26) | AGGGGATTCTACAGCATGACTGAGC | UACUAC, −7_1-1_wobble_U-G, 0_1- | | |
| | | | AGTCCCGTAGTCGGCAATCTTTGCA | 1_mismatch_A-C, 2_1-1_mismatch_C-C, 4_1- | | |
| | | | TACTACGCAGCATTGGGATACAGTG | 1_wobble_U-G, 13_2−2_bulge-symmetric_AC-UG, | | |
| | | | TGAAAAGCAGCA | 26_6-6_internal_loop-symmetric_GGGGAU- | | |
| | | | | UAGGGG | | |
| 185 | 25 | ML exhaustive_ADAR1/2_ | CCCTGGTGTGCCCTCTGATGTTTTTT | −20_6-6_internal_loop-symmetric_CAUCAU- | −20 | 26 |
| | | 1042 (−20, +26) | AGGGGATTCTACAGCAATACTCAGC | UACUAC, −13_1-1_mismatch_G-A, 0_1- | | |
| | | | AGTTCCGTAGTCAGCAATATTTGCA | 1_mismatch_A-C, 2_1-1_mismatch_C-U, 4_1- | | |
| | | | TACTACGCAGCATTGGGATACAGTG | 1_wobble_U-G, 9_1-1_mismatch_C-C, 14 1- | | |
| | | | TGAAAAGCAGCA | 1_mismatch_C-A, 26_6-6_internal_loop- | | |
| | | | | symmetric_GGGGAU-UAGGGG | | |
| 186 | 26 | ML_generative_0002 | CCCTGGTGTGCCCTCTGATGTTTTTT | −20_6-6_internal_loop-symmetric_CAUCAU- | −20 | 26 |
| | | (−20, +26) | AGGGGATTCTAGAGAGGTACTGTGC | UACUAC, −10_1-1_mismatch_U-C, −7_1- | | |
| | | | CATCCCGTGGTCGGCCATCTTTGCA | 1_wobble_U-G, −3 1-1_wobble_U-G, 0_1- | | |
| | | | TACTACGCAGCATTGGGATACAGTG | 1_mismatch_A-C, 2 1-1_mismatch_C-C, 5_1- | | |
| | | | TGAAAAGCAGCA | 1_mismatch_U-C, 8 1-1_mismatch_U-U, 15 1- | | |
| | | | | 1_wobble_U-G, 16_1-1_mismatch_G-A, 19 1- | | |
| | | | | 1_mismatch_G-G, 26_6-6_internal_loop- | | |
| | | | | symmetric GGGGAU-UAGGGG | | |
| 187 | 26 | ML_generative_0013 | CCCTGGTGTGCCCTCTGATGTTTTTT | −20_6-6_internal_loop-symmetric_CAUCAU- | −20 | 26 |
| | | (−20, +26) | AGGGGATTCTAGAGCAGGACGGTG | UACUAC, −7_1-1_wobble_U-G, −3_1- | | |
| | | | CAGTCCCGTGGTCGGCAATCTTTGC | 1_wobble_U-G, 0_1-1_mismatch_A-C, 2_1- | | |
| | | | ATACTACGCAGCATTGGGATACAGT | 1_mismatch_C-C, 4_1-1_wobble_U-G, 8_1- | | |
| | | | GTGAAAAGCAGCA | 1_mismatch_U-U, 10_1-1_mismatch_A-G, 13_1- | | |
| | | | | 1_mismatch_A-G, 19_1-1_mismatch_G-G, 26_6- | | |
| | | | | 6_internal_loop-symmetric_GGGGAU-UAGGGG | | |
| 188 | 27 | ML_generative_0016 | CCCTGGTGTGCCCTCTGATGTTTTTT | −20_6-6_internal_loop-symmetric_CAUCAU- | −20 | 26 |
| | | (−20, +26) | AGGGGATTCTACAGGAGGACTGGG | UACUAC, −10_2−2_bulge-symmetric_UU-CU, | | |
| | | | CAGTCCCGTGGTCGCCCTTCTTTGCA | −8_1-1_mismatch_C-C, -7_1-1_wobble_U-G, −3_1- | | |
| | | | TACTACGCAGCATTGGGATACAGTG | 1_wobble_U-G, 0_1-1_mismatch_A-C, 2_1- | | |
| | | | TGAAAAGCAGCA | 1_mismatch_C-C, 4_1-1_wobble_U-G, 8 1- | | |
| | | | | 1_wobble_U-G, 13_1-1_mismatch_A-G, 16_1- | | |
| | | | | 1_mismatch_G-G, 26_6-6_internal_loop- | | |
| | | | | symmetric_GGGGAU-UAGGGG | | |
| 189 | 27 | ML_generative_0043 | CCCTGGTGTGCCCTCTGATGTTTTTT | −20_6-6_internal_loop-symmetric_CAUCAU- | −20 | 26 |
| | | (−20, +26) | AGGGGATTCTACAGCGGTACTGTGC | UACUAC, −10_1-1_mismatch_U-C, −9_1- | | |
| | | | AAATCCGTGGTCGGTCATCTTTGCA | 1_wobble_G-U, −7_1-1_wobble_U-G, −3_1- | | |
| | | | TACTACGCAGCATTGGGATACAGTG | 1_wobble_U-G, 0_1-1_mismatch_A-C, 2 2- | | |
| | | | TGAAAAGCAGCA | 2_bulge-symmetric_CA-AU, 8_1-1_mismatch_U- | | |
| | | | | U, 15_1-1_wobble_U-G, 26_6-6_internal_loop- | | |
| | | | | symmetric GGGGAU-UAGGGG | | |
| 190 | 28 | ML_generative_0058 | CCCTGGTGTGCCCTCTGATGTTTTTT | −20_6-6_internal_loop-symmetric_CAUCAU- | −20 | 26 |
| | | (−20, +26) | AGGGGATTCTACGGCAGGACTGGG | UACUAC, −10_1-1_mismatch_U-C, −7 1- | | |
| | | | GAAATCCGTGGTCGGCCATCTTTGC | 1_wobble_U-G, −3_1-1_wobble_U-G, 0_1- | | |
| | | | ATACTACGCAGCATTGGGATACAGT | 1_mismatch_A-C, 2_2−2_bulge-symmetric_CA- | | |
| | | | GTGAAAAGCAGCA | AU, 6_1-1_mismatch_G-G, 8_1-1_wobble_U-G, | | |
| | | | | 13_1-1_mismatch_A-G, 18_1-1_wobble_U-G, | | |
| | | | | 26_6-6_internal_loop-symmetric_GGGGAU- | | |
| | | | | UAGGGG | | |
| 191 | 28 | ML_generative_0071 | CCCTGGTGTGCCCTCTGATGTTTTTT | −20_6-6_internal_loop-symmetric_CAUCAU- | −20 | 26 |
| | | (−20, +26) | AGGGGATTCTACAGCGGTACTGCGC | UACUAC, −10_1-1_mismatch_U-C, −8_1- | | |
| | | | AGTCCCGTGGTGGTCCATCTTTGCA | 1_mismatch_C-U, −7_1-1_wobble_U-G, −6_1- | | |
| | | | TACTACGCAGCATTGGGATACAGTG | 1_mismatch_G-G, −3_1-1_wobble_U-G, 0_1- | | |
| | | | TGAAAAGCAGCA | 1_mismatch_A-C, 2_1-1_mismatch_C-C, 4_1- | | |
| | | | | 1_wobble_U-G, 8_1-1_mismatch_U-C, 15_1- | | |
| | | | | 1_wobble_U-G, 26_6-6_internal_loop- | | |
| | | | | symmetric_GGGGAU-UAGGGG | | |
| 192 | 29 | ML_generative_0130 | CCCTGGTGTGCCCTCTGATGTTTTTT | −20_6-6_internal_loop-symmetric_CAUCAU- | −20 | 26 |
| | | (−20, +26) | AGGGGATTCTATAGCAGGAATGAG | UACUAC, −7_1-1_wobble_U-G, −3_1- | | |
| | | | GAAATCCGTGGTCGGCAATCTTTGC | 1_wobble_U-G, 0_1-1_mismatch_A-C, 2_2- | | |
| | | | ATACTACGCAGCATTGGGATACAGT | 2_bulge-symmetric_CA-AU, 6_1-1_mismatch_G- | | |
| | | | GTGAAAAGCAGCA | G, 11_3−3_bulge-symmetric_GUA-GAA, 19_1- | | |
| | | | | 1_wobble_G-U, 26_6-6_internal_loop- | | |
| | | | | symmetric_GGGGAU-UAGGGG | | |
| 193 | 29 | ML_generative_0156 | CCCTGGTGTGCCCTCTGATGTTTTTT | −20_6-6_internal_loop-symmetric_CAUCAU- | −20 | 26 |
| | | (−20, +26) | AGGGGATTCTACAGCAGTACTCAGG | UACUAC, −9_2−2_bulge-symmetric_UG-AU, | | |
| | | | AAATCCGTGGTCGGATATCTTTGCA | −7_1-1_wobble_U-G, −3_1-1_wobble_U-G, 0_1- | | |
| | | | TACTACGCAGCATTGGGATACAGTG | 1_mismatch_A-C, 2_2−2_bulge-symmetric_CA- | | |
| | | | TGAAAAGCAGCA | AU, 6_1-1_mismatch_G-G, 9_1-1_mismatch_C-C, | | |
| | | | | 26_6-6_internal_loop-symmetric_GGGGAU- | | |
| | | | | UAGGGG | | |
| 194 | 30 | ML_generative_0176 | CCCTGGTGTGCCCTCTGATGTTTTTT | −20_6-6_internal_loop-symmetric_CAUCAU- | −20 | 26 |
| | | (−20, +26) | AGGGGATTCTAAAGCAGGAGTGAG | UACUAC, −7_1-1_wobble_U-G, −3_1- | | |
| | | | CAGATCCGTGGTCGGCAATCTTTGC | 1_wobble_U-G, 0->3_4−4_bulge- | | |
| | | | ATACTACGCAGCATTGGGATACAGT | symmetric_AGCA-AUCC, 4_1-1_wobble_U-G, | | |
| | | | GTGAAAAGCAGCA | 11_1-1_mismatch_G-G, 13_1-1_mismatch_A-G, | | |
| | | | | 19_1-1_mismatch_G-A, 26_6-6_internal_loop- | | |
| | | | | symmetric_GGGGAU-UAGGGG | | |
| 195 | 30 | ML_generative_0218 | CCCTGGTGTGCCCTCTGATGTTTTTT | −20_6-6_internal_loop-symmetric_CAUCAU- | −20 | 26 |
| | | (−20, +26) | AGGGGATTCTACAGGTGTACTGTGA | UACUAC, −8_2−2_bulge-symmetric_GC-UA, | | |
| | | | AATCCCGTGGTCATAAATCTTTGCA | −3_1-1_wobble_U-G, 0_1-1_mismatch_A-C, 2_1- | | |
| | | | TACTACGCAGCATTGGGATACAGTG | 1_mismatch_C-C, 6_1-1_mismatch_G-A, 8_1- | | |
| | | | TGAAAAGCAGCA | 1_mismatch_U-U, 15_2−2_bulge-symmetric_UG- | | |
| | | | | GU, 26_6-6_internal_loop-symmetric_GGGGAU- | | |
| | | | | UAGGGG | | |
| 196 | 31 | ML_exhaustive_ADAR1/2_ | CCCTGGTGTGCCCTCTGATGTTTTTT | −20_6-6_internal_loop-symmetric_CAUCAU- | −20 | 26 |
| | | 1045 (−20, +26) | AGGGGATTCTACAGCATTACTCAGC | UACUAC, −13_1-1_mismatch_G-A, 0_1- | | |
| | | | AGTCCCGTAGTCAGCAATATTTGCA | 1_mismatch_A-C, 2_1-1_mismatch_C-C, 4_1- | | |
| | | | TACTACGCAGCATTGGGATACAGTG | 1_wobble_U-G, 9_1-1_mismatch_C-C, 14 1- | | |
| | | | TGAAAAGCAGCA | 1_mismatch_C-U, 26_6-6_internal_loop- | | |
| | | | | symmetric_GGGGAU-UAGGGG | | |
| 197 | 31 | ML_exhaustive_ADA | CCCTGGTGTGCCCTCTGATGTTTTTT | −20_6-6_internal_loop-symmetric_CAUCAU- | −20 | 26 |
| | | R1/2_1540 (−20, +26) | AGGGGATTCTACAGCATTACTCAGC | UACUAC, −9_1-1_mismatch_G-A, 0_1- | | |
| | | | AAATCCGTAGTCAGAAATCTTTGCA | 1_mismatch_A-C, 2_2−2_bulge-symmetric_CA- | | |
| | | | TACTACGCAGCATTGGGATACAGTG | AU, 9_1-1_mismatch_C-C, 14_1-1_mismatch_C- | | |
| | | | TGAAAAGCAGCA | U, 26_6-6_internal_loop-symmetric_GGGGAU- | | |
| | | | | UAGGGG | | |
| 198 | 32 | ML exhaustive ADAR1_ | CCCTGGTGTGCCCTCTGATGTTTTTT | −20_6-6_internal_loop-symmetric_CAUCAU- | −20 | 26 |
| | | 0315 (−20, +26) | AGGGGATTCTACAGCAGGACTGAGC | UACUAC, −13_1-1_mismatch_G-A, −3_1- | | |
| | | | AATCTTGTGGTCAGCAATATTTGCA | 1_wobble_U-G, 1_1-1_wobble_G-U, 2 1- | | |
| | | | TACTACGCAGCATTGGGATACAGTG | 1_mismatch_C-C, 13_1-1_mismatch_A-G, 26_6- | | |
| | | | TGAAAAGCAGCA | 6 internal_loop-symmetric_GGGGAU-UAGGGG | | |
| 199 | 32 | ML_exhaustive_ADAR1_ | CCCTGGTGTGCCCTCTGATGTTTTTT | −20_6-6_internal_loop-symmetric_CAUCAU- | −20 | 26 |
| | | 0414 (−20, +26) | AGGGGATTCTACAGCAGGACTGAGC | UACUAC, −13_1-1_mismatch_G-A, −3_1- | | |
| | | | AATGGAGTGGTCAGCAATATTTGCA | 1_wobble_U-G, 0->1_2−2_bulge-symmetric_AG- | | |
| | | | TACTACGCAGCATTGGGATACAGTG | GA, 13_1-1_mismatch_A-G, 26_6-6_internal_loop- | | |
| | | | TGAAAAGCAGCA | symmetric_GGGGAU-UAGGGG | | |
| 200 | 33 | ML_exhaustive_ADAR2_ | CCCTGGTGTGCCCTCTGATGTTTTTT | −20_6-6_internal_loop-symmetric_CAUCAU- | −20 | 26 |
| | 36 | 0013 (−20, +26) | AGGGGATTCTACAGCACGACTGAGC | UACUAC, −10_1-1_mismatch_U-C, −1->0_2- | | |
| | | | AGTGCGTTAGTCAGCCATCTTTGCA | 2_bulge-symmetric_CA-GU, 4_1-1_wobble_U-G, | | |
| | | | TACTACGCAGCATTGGGATACAGTG | 13_2−2_bulge-symmetric_AC-CG, 26_6- | | |
| | | | TGAAAAGCAGCA | 6_internal_loop-symmetric_GGGGAU-UAGGGG | | |

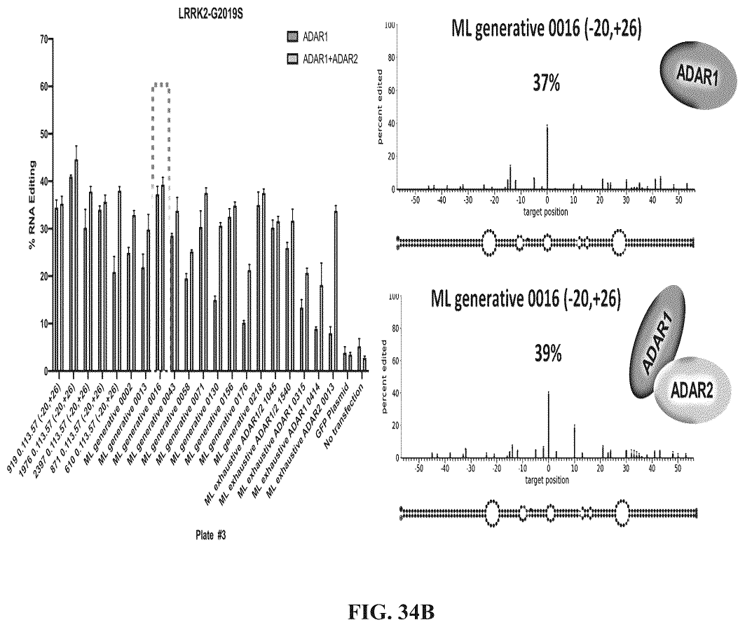

FIG. 34A and FIG. 34B shows selection of two exemplary engineered guide RNAs displaying superior editing that were selected for further engineering.

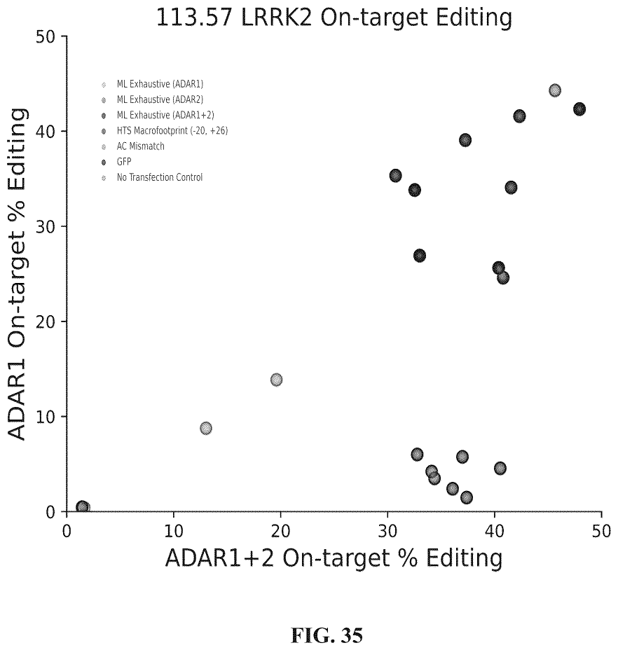

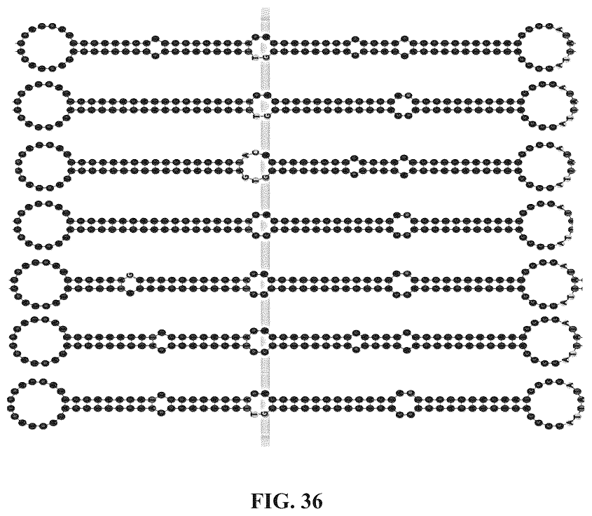

Of note, the machine learning algorithm provided an accurate prediction of ADAR specificity. As shown in FIG. 35, guide RNAs selected for ADAR1, ADAR2, or ADAR1 and ADAR2 displayed specificity for the appropriate ADAR enzyme in vitro. FIG. 36 depicts engineered guide RNA designs that showed specificity for ADAR2 in FIG. 35. In this system, engineered guide RNAs designed to form an A-G mismatch at the target adenosine exhibited facilitating preferential RNA editing by ADAR2.

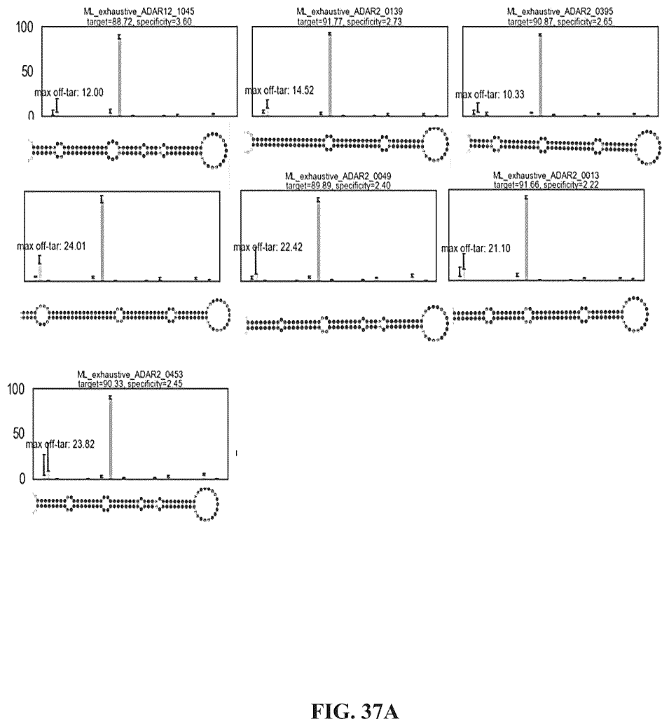

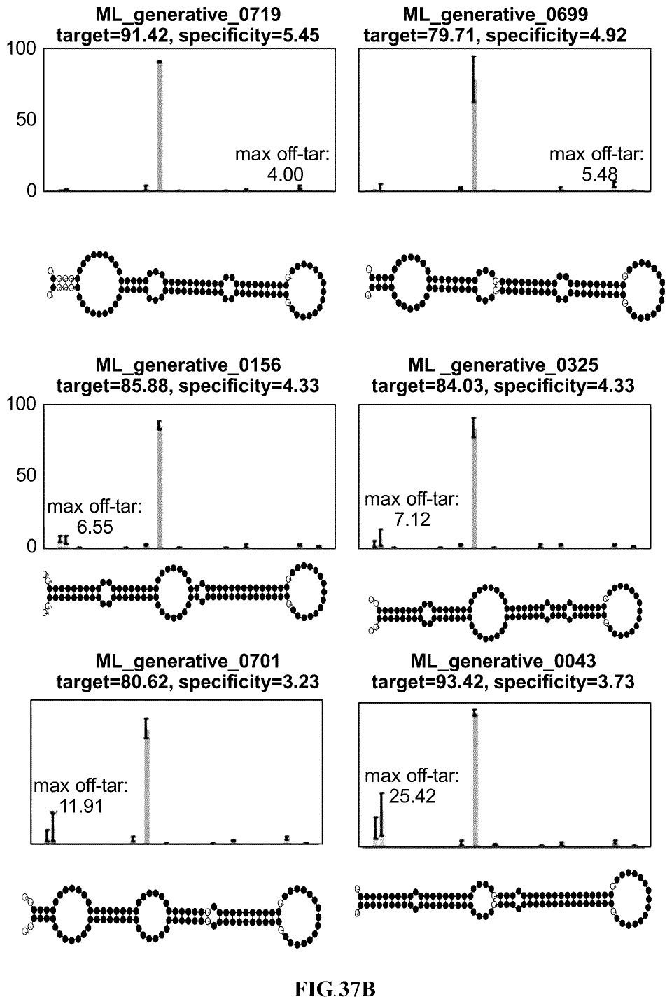

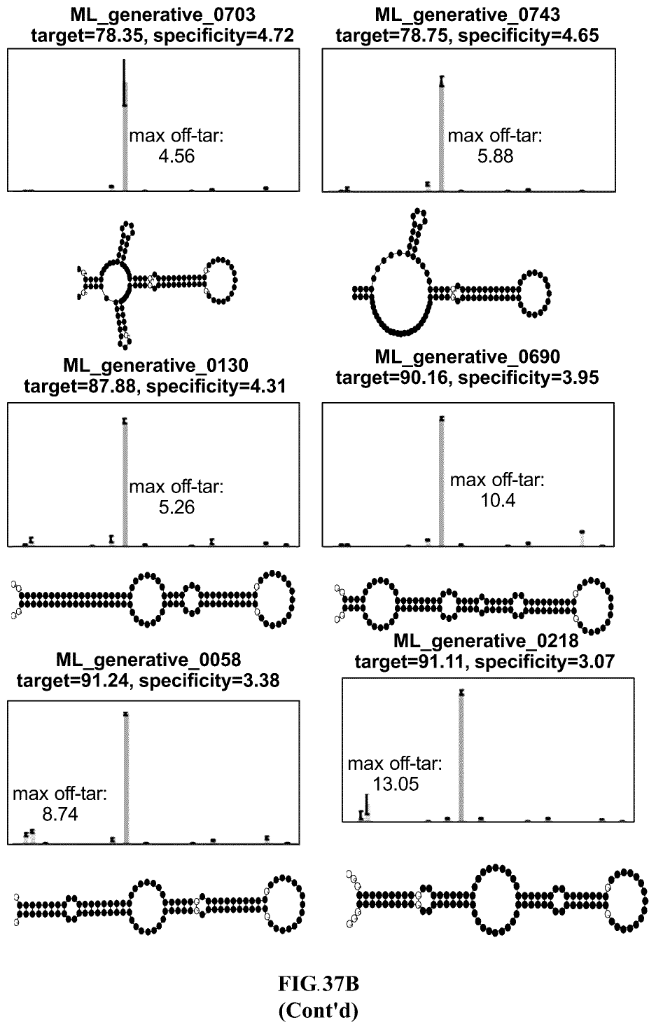

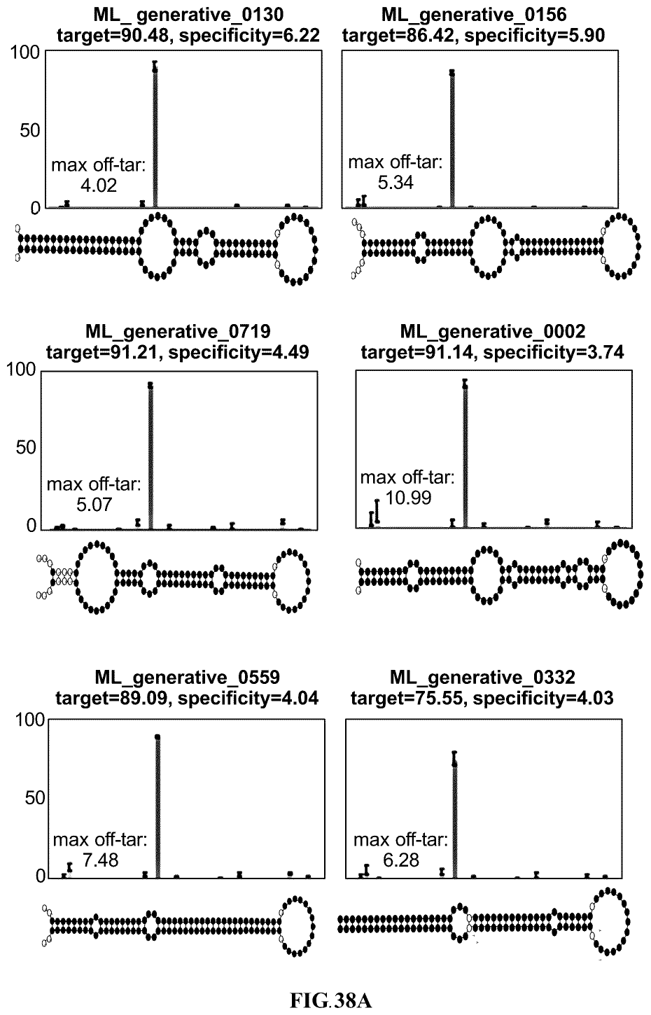

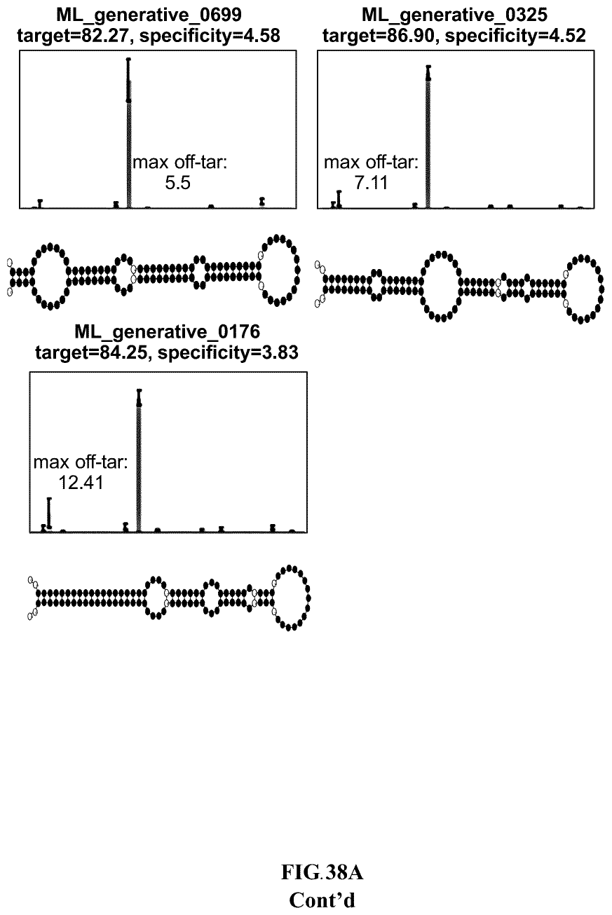

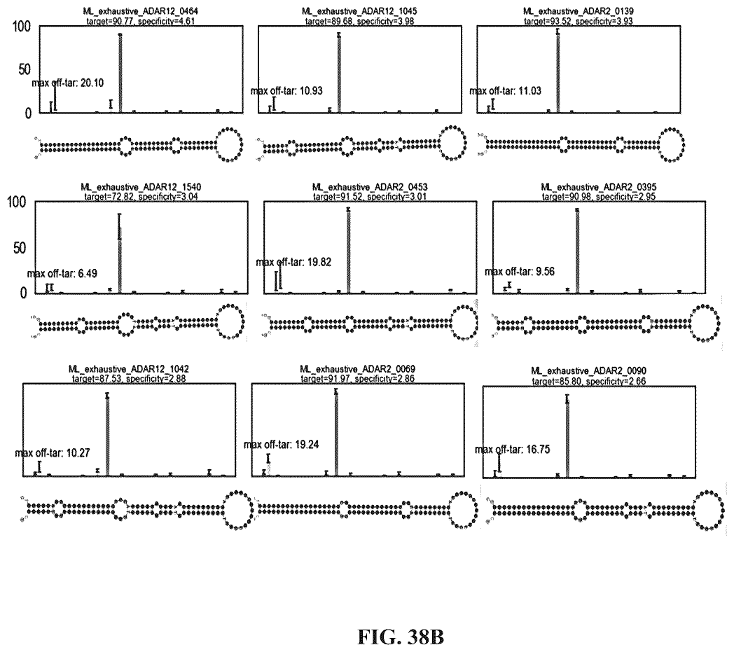

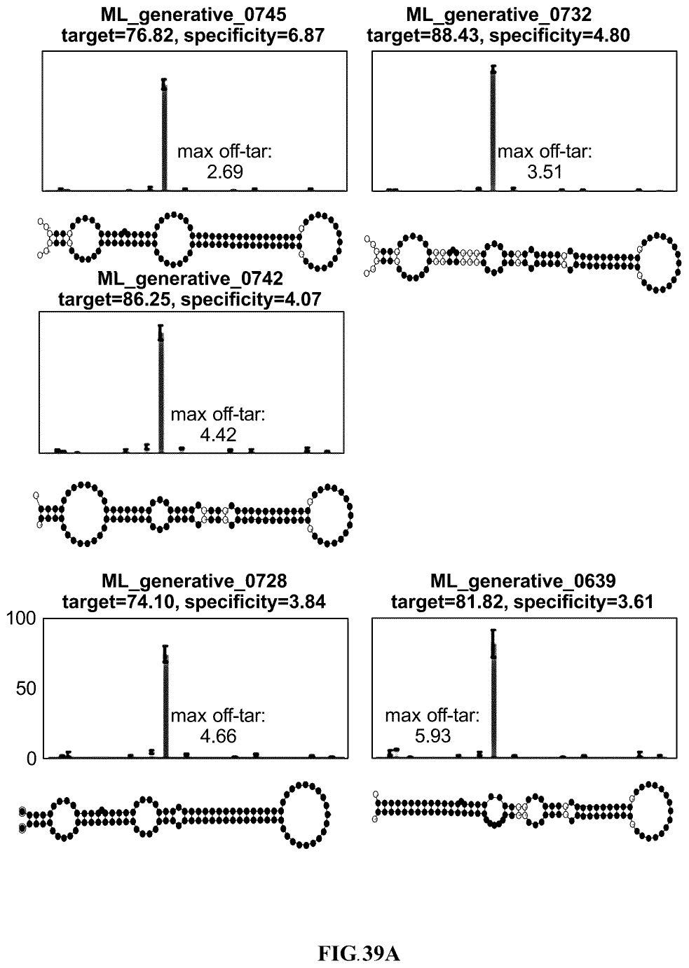

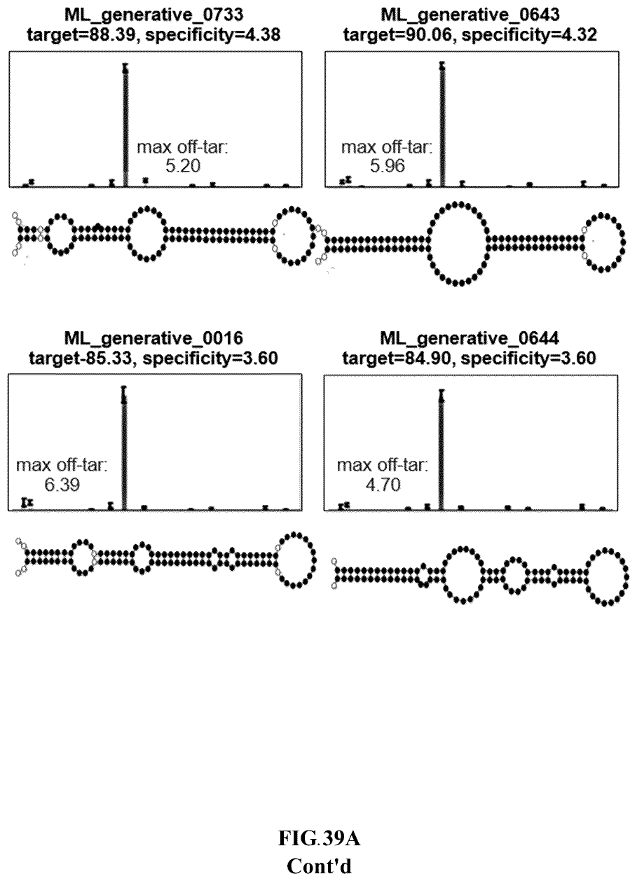

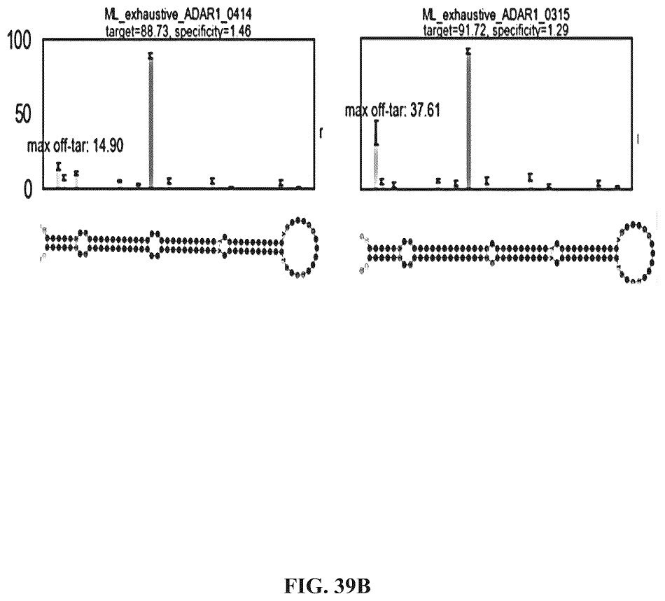

FIG. 37A and FIG. 37B show the top performing engineered guide RNAs that display specificity for ADAR1+ADAR2. FIG. 38A and FIG. 38B show the top performing engineered guide RNAs that display specificity for ADAR2. FIG. 39A and FIG. 39B show the top performing engineered guide RNAs that display specificity for ADAR1.

### Example 8

ML gRNAs Targeting LRRK2

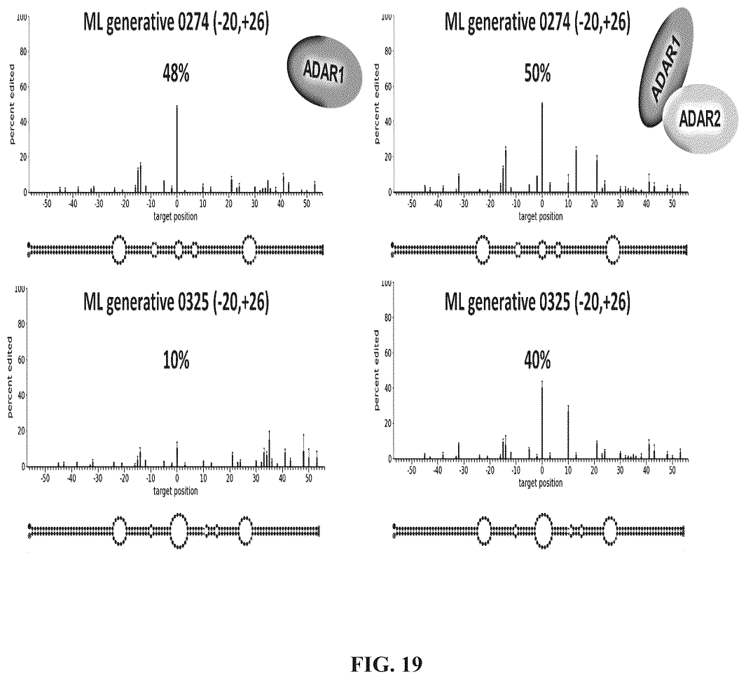

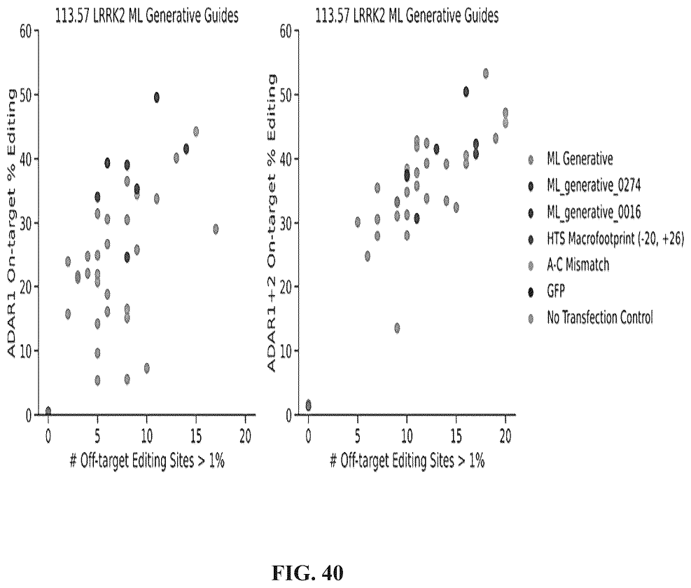

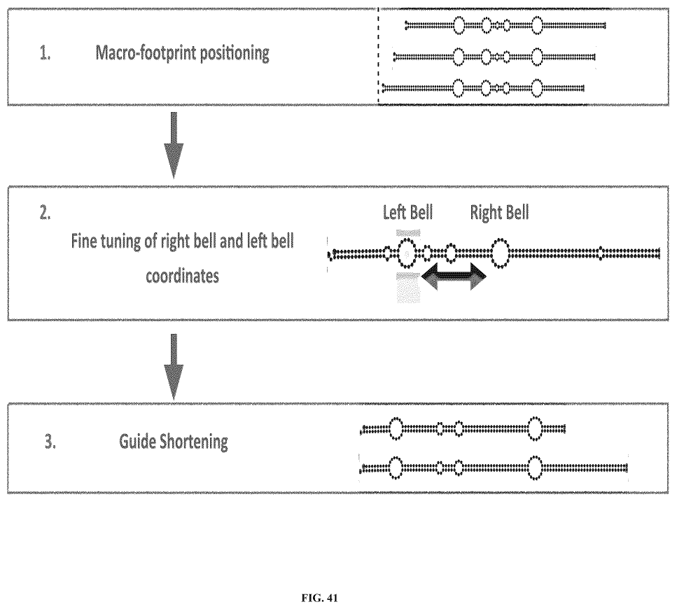

This example describes machine learning (ML)-derived gRNAs targeting LRRK2. Two machine learning model types were utilized, a generative model and an exhaustive model, to engineer LRRK2 gRNAs that were subsequently evaluated. Next-generation sequencing (NGS) was used to compare highly efficient and specific ML-derived gRNAs and gRNAs generated using in vitro high throughput screening (HTS) methods. gRNAs were dosed in HEK293 cells expressing a LRRK2 cDNA minigene. Two generative ML gRNAs, in particular, leveraged ADAR to facilitate highly efficient and specific RNA editing (FIG. 27—CCCTGGTGTGCCCTCTGATGTTTTTTAGGGGATTCTACAGGAGGACTGGGCAGTCCCGTGGT CGCCCTTCTIGCATACTACGCAGCATGGGATACAGTGTGAAAAGCAGCA (SEQ ID NO: 201), FIG. 19—CCCTGGTGTGCCCTCTGATGTTTTTTAGGGGATTCTACAGCAGTACTGTCCAGTCCCGTGGTC GTAAATCTTGCATACTACGCAGCATGGGATACAGTGTGAAAAGCAGCA (SEQ ID NO: 202), and FIG. 40).

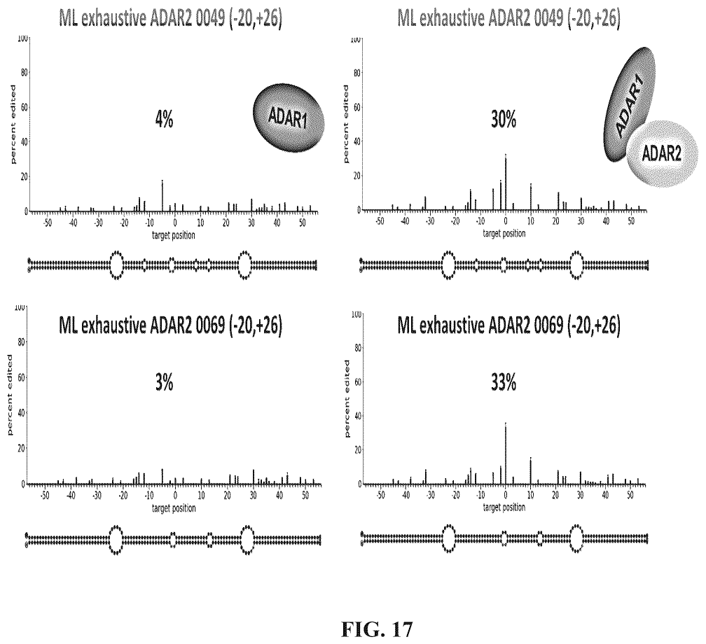

In addition, gRNAs that preferentially leverage ADAR2 for RNA editing are also disclosed herein (e.g., FIG. 17—CCCTGGTGTGCCCTCTGATGTTTTTTAGGGGATCTACAGCACGACTGAGCAGTGCGTAGTC GGCAATCTTGCATACTACGCAGCATTGGGATACAGTGTGAAAAGCAGCA (SEQ ID NO: 203)). FIG. 35 shows a plot of ADAR1+2% on-target editing (x-axis) versus ADAR1-only % on-target editing (y-axis). As shown in this figure, several gRNAs of the present disclosure (structures of the guide-target RNA scaffold shown in FIG. 36), that comprise an A-G mismatch, show an ADAR2 preference. Thus, an A-G mismatch at the target A may potentially drive ADAR2-specific editing.

### Example 9

### Engineering of LRRK2 Guide RNAs Selected Using HTS

This example describes engineering of guide RNAs using a high throughput screen (see EXAMPLE 1) for targeting of the LRRK2 G2019S mutation for RNA editing by ADAR. FIG. 41 provides an overview of the engineering process. As depicted in FIG. 41, the engineering process includes: 1. positioning of the macro-footprint; 2. fine-tuning of the left-barbell and right-barbell coordinates; and 3. shortening of the guide length. LRRK2 guide RNAs count610 (−14, 26)—SEQ ID NO: 5, count871 (−16, 24)—SEQ ID NO: 23, and count919 (−12, 24)—SEQ ID NO: 50 were selected for further engineering.

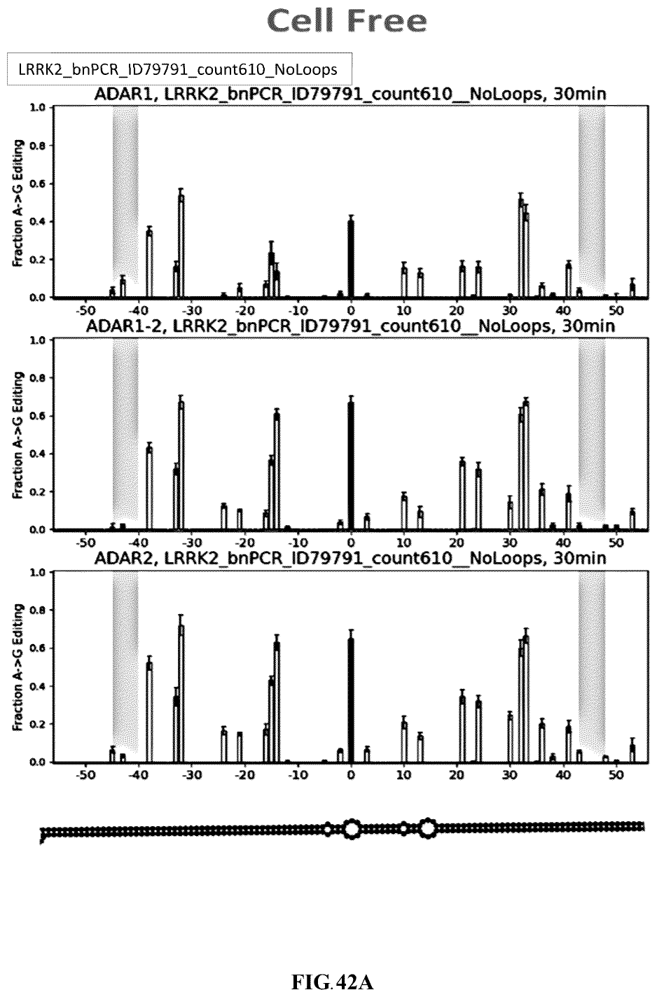

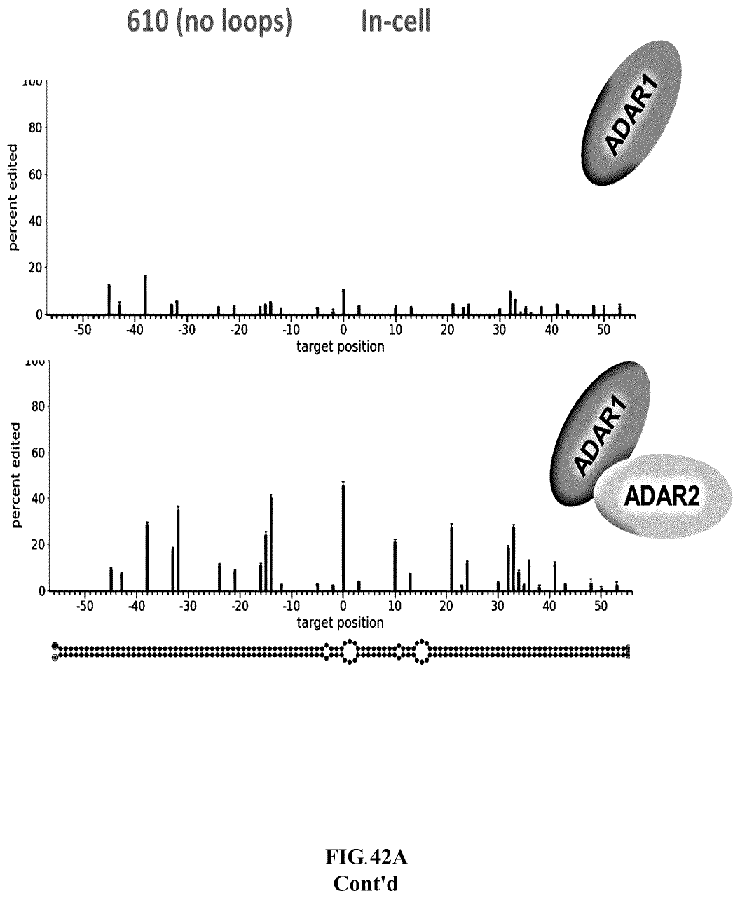

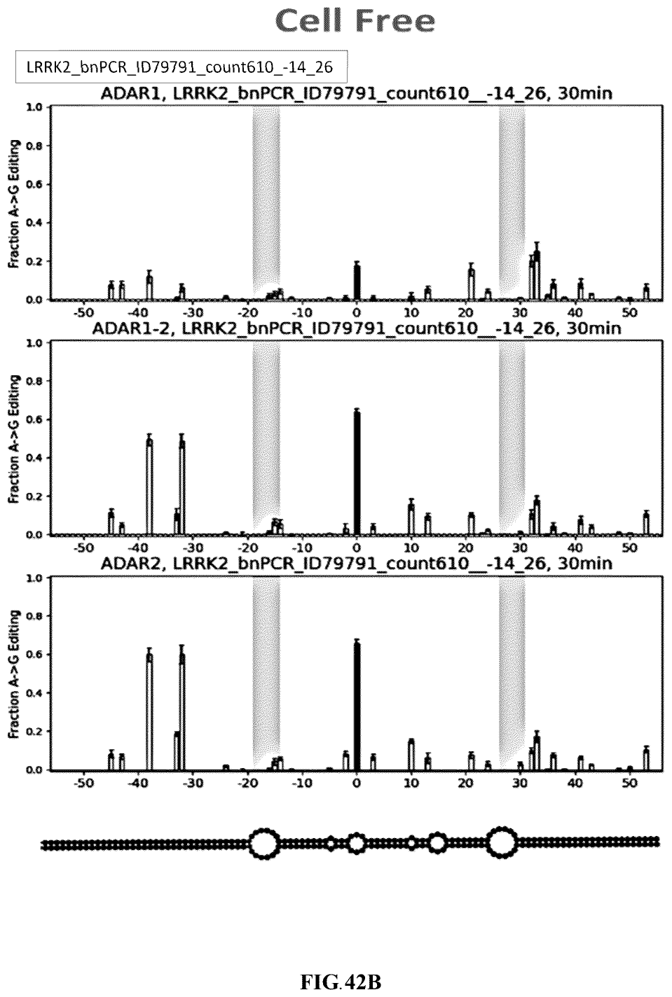

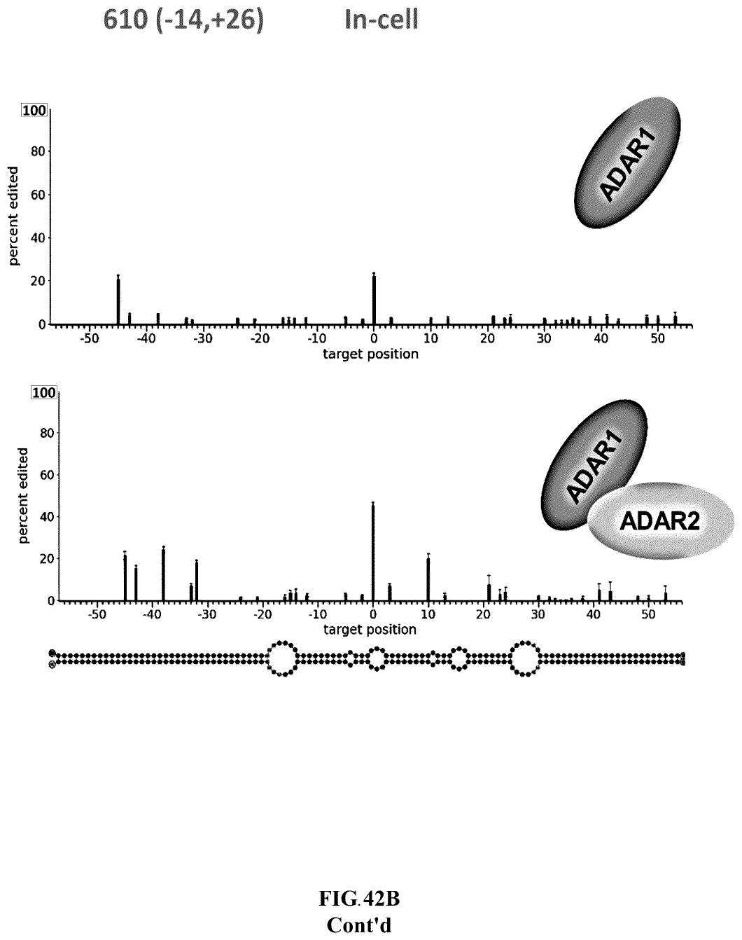

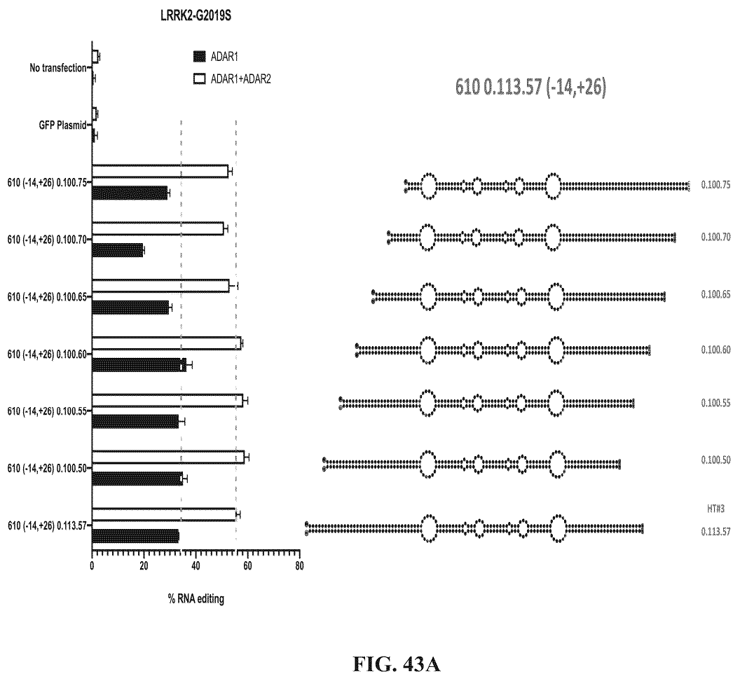

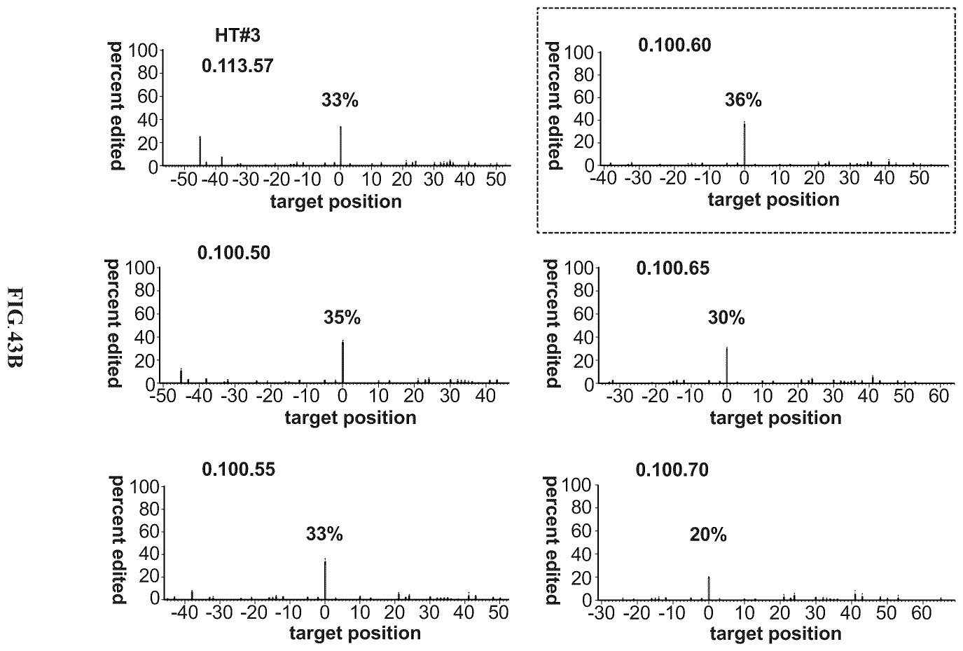

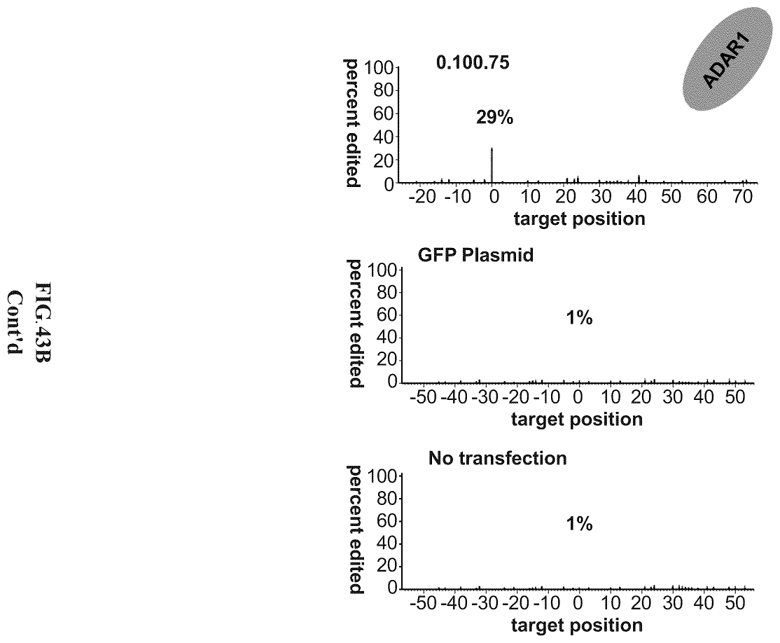

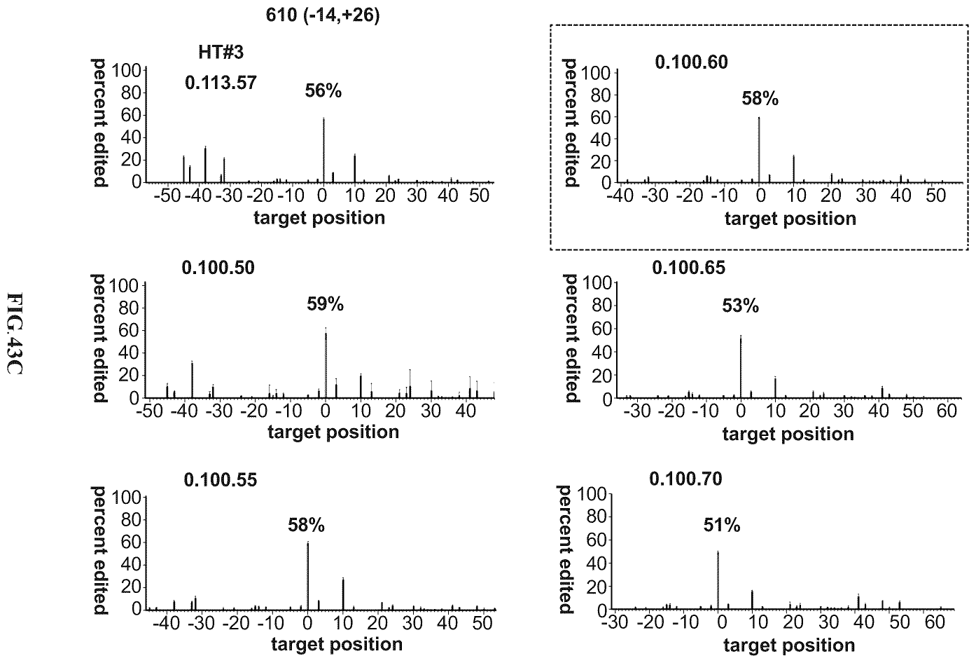

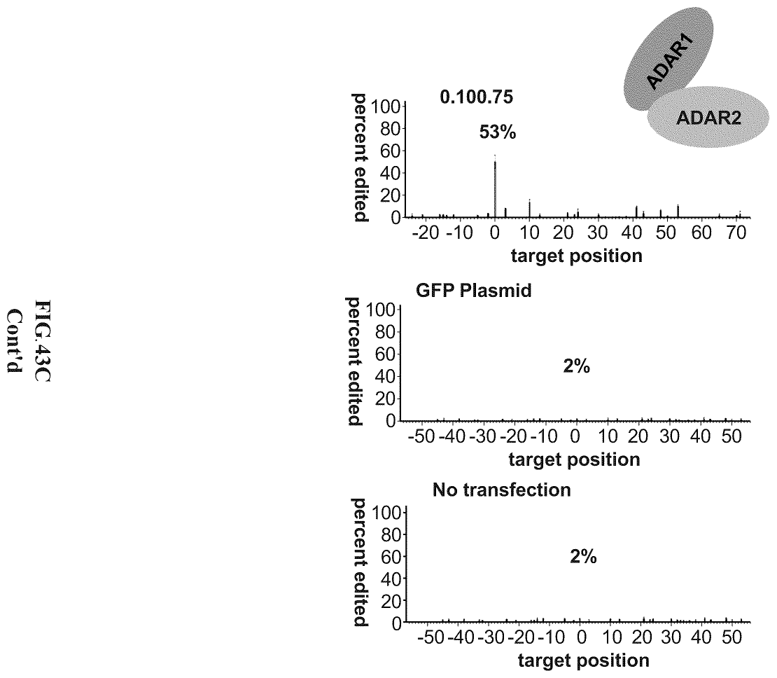

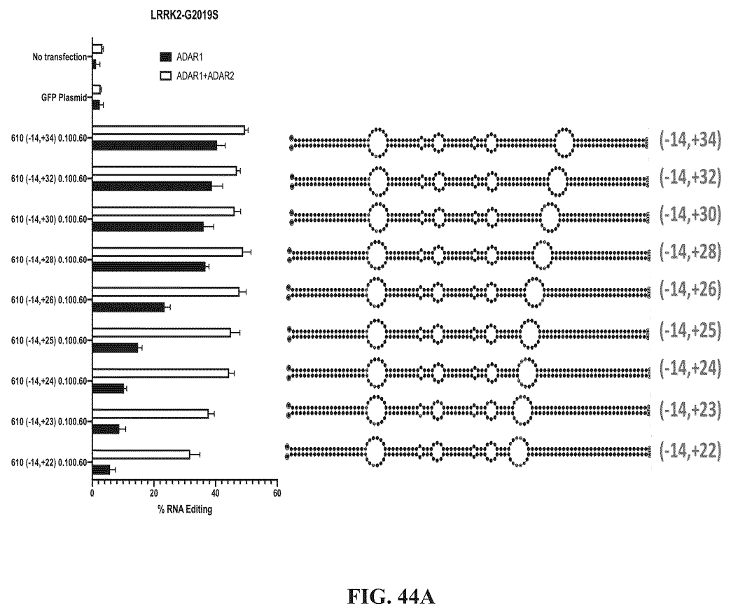

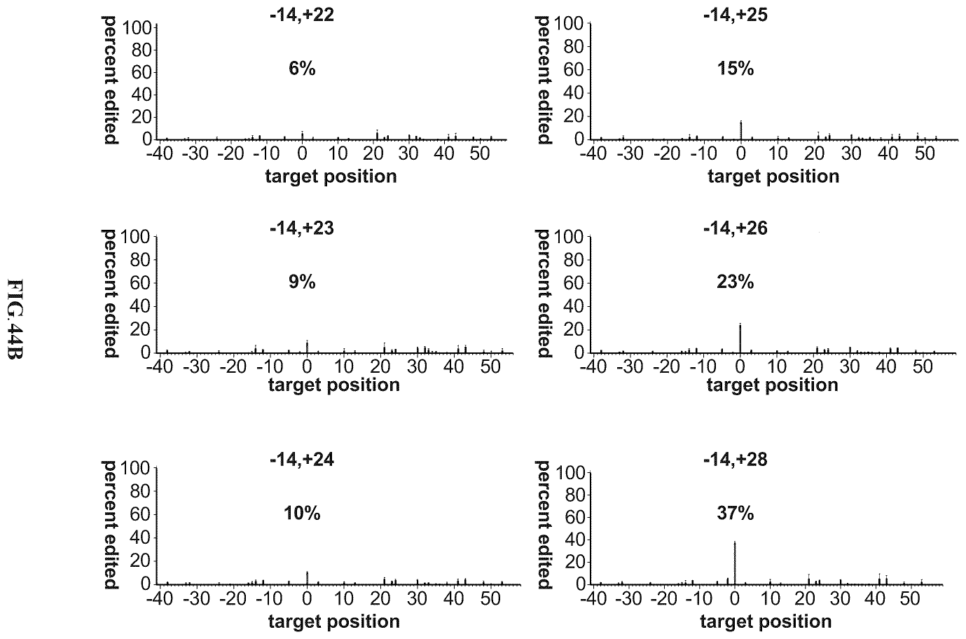

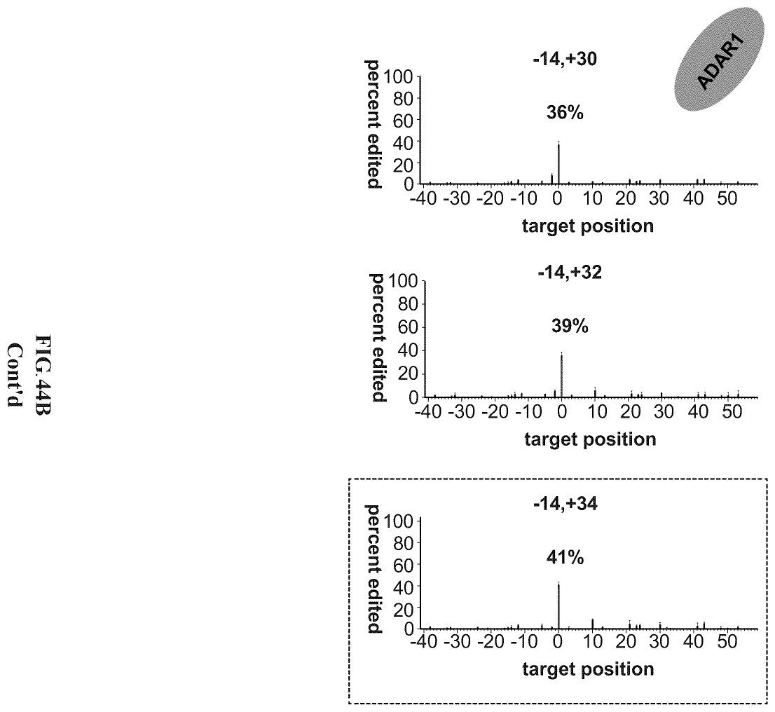

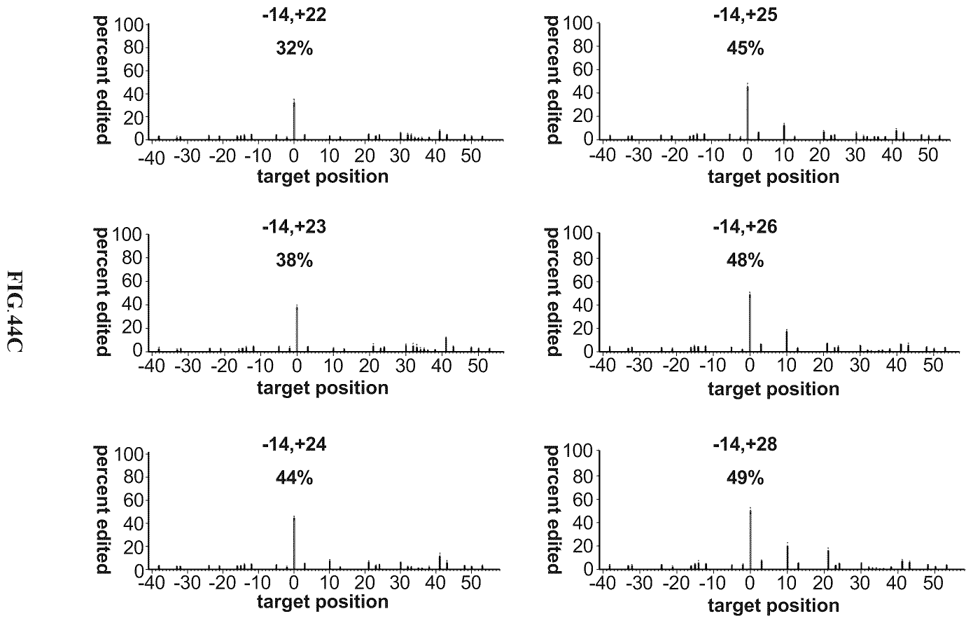

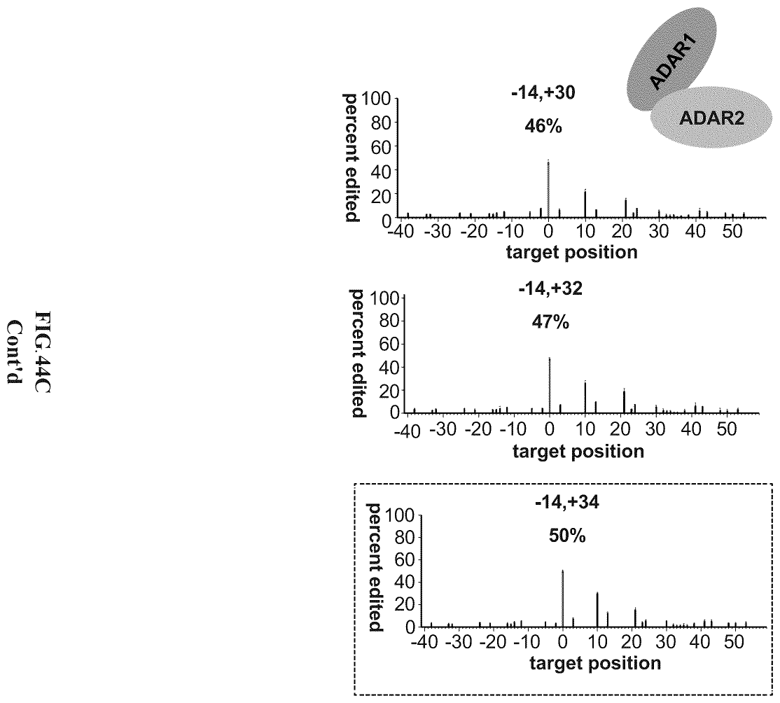

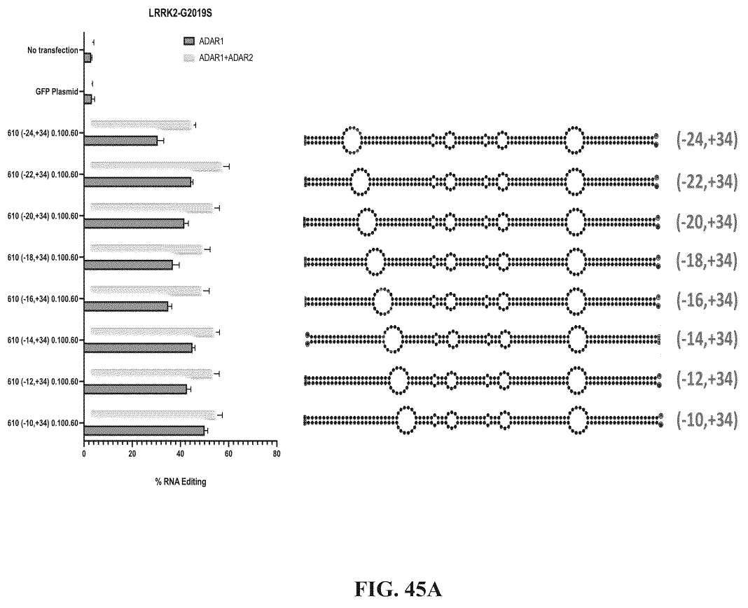

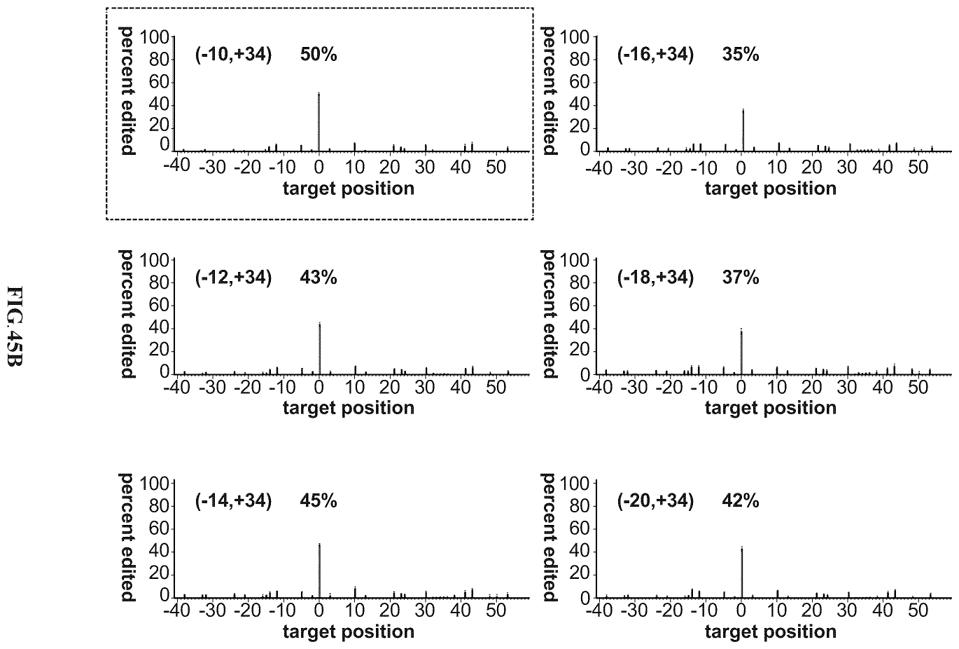

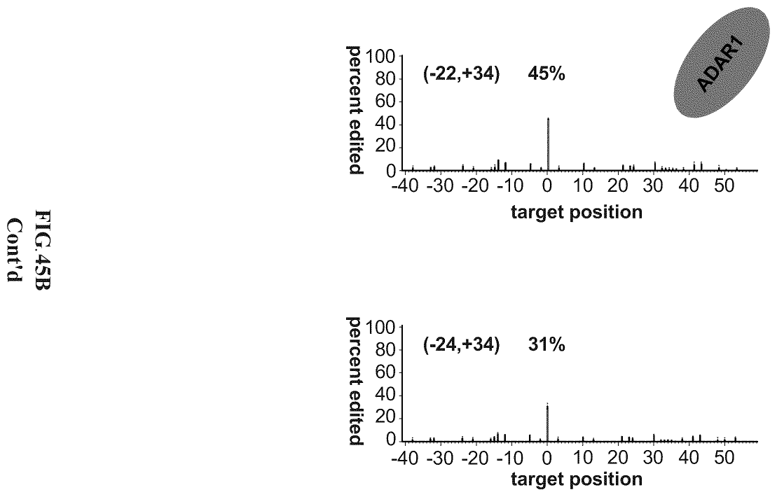

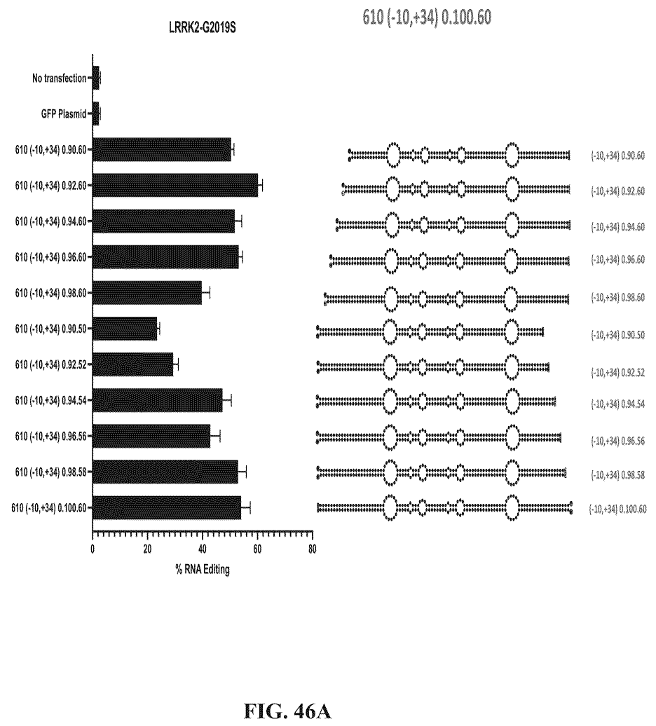

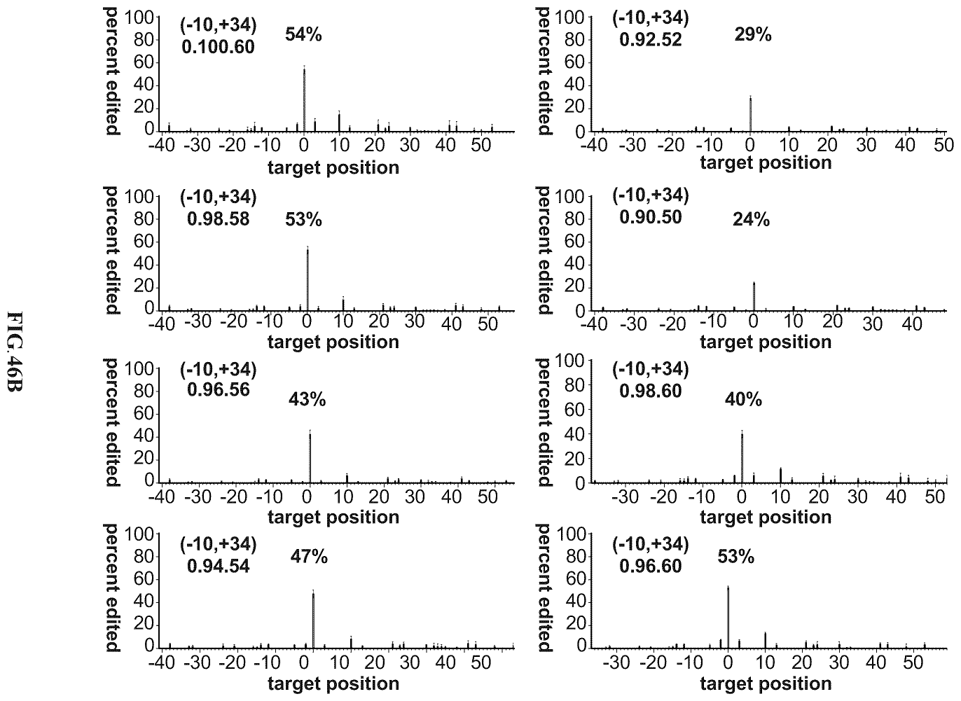

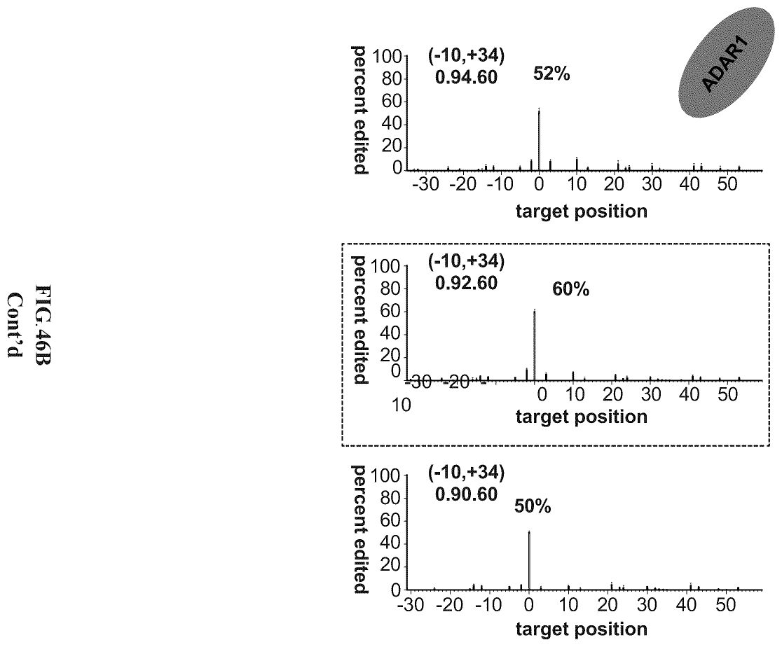

The addition of barbell macro-footprints formed in the guide-target RNA scaffold results in an increase in on-target adenosine editing relative to the amount of off-target editing. As demonstrated in FIG. 42A and FIG. 42B, guide610 (forms a barbell macro-footprint upon hybridization to target RNA with barbells at position −14, +26) displayed a reduction in off-target editing for both ADAR1 and ADAR1+ADAR2, relative to the same engineered guide RNAs lacking the latent structure that would result in a barbell macro-footprint upon hybridization to target RNA.

FIG. 43A-FIG. 43C depict the first step of the design process for guide 610: positioning of the macro-footprint. FIG. 43A shows tiling of the macro-footprint positioning within the guide-target RNA scaffold for guide610 with respect to the A/C mismatch and how this tiling affects RNA editing by ADAR1 and ADAR1+ADAR2. FIG. 43B shows the percent editing for the tiled guide610 variants via ADAR1. As noted, the engineered guide RNA with the mismatch positioned 60 nucleotides (0.100.60) from the end of the guide, displaying a LRRK2 editing of 36%, was selected for further engineering. FIG. 43C shows the percent editing for the tiled guide610 variants via ADAR1+ADAR2. As noted, the engineered guide RNA with the mismatch positioned 60 nucleotides (0.100.60) from the end of the engineered guide RNA, displaying a LRRK2 editing of 58%, was selected for further engineering.

The 0.100.60 guide610 was carried into the next step of design: fine-tuning of the left-barbell and right-barbell coordinates. FIG. 44A-FIG. 44C show engineering of the right barbell coordinates. As shown in FIG. 44A, the coordinate of the right barbell was tiled between the following coordinates with respect to the A/C mismatch: +22, +23, +24, +25, +26, +28, +30, +32, and +34, and the effect of each position on ADAR1 and ADAR1+ADAR2 editing was determined. FIG. 44B shows the percent editing for the tiled guide610 variants via ADAR1. As noted, the guide with the right barbell at position +34 (with respect to the A/C mismatch), displaying a LRRK2 editing of 41%, was selected for further engineering. FIG. 44C shows the percent editing for the tiled guide610 variants via ADAR1+ADAR2. As noted, the guide with the right barbell at position +34 (with respect to the A/C mismatch), displaying a LRRK2 editing of 50% via ADAR, was selected for further engineering.

The 0.100.60 guide610 having a right barbell at position +34 (with respect to the A/C mismatch) was utilized as a starting scaffold for left-barbell coordinate tiling. FIG. 45A and FIG. 45B show engineering of the left barbell coordinates. As shown in FIG. 45A, the coordinate of the left barbell was tiled between the following coordinates with respect to the A/C mismatch: −10, −12, −14, −16, −18, −20, −22, and −24, and the effect of each position on ADAR1 and ADAR1+ADAR2 editing was determined. FIG. 45B shows the percent editing for the tiled guide610 variants via ADAR1. As noted, the guide with the left barbell at position −10 and right barbell at position +34 (with respect to the A/C mismatch), displaying a LRRK2 editing of 50% via ADAR, was selected for further engineering.

The guide610 variant having barbell coordinates at (−10, +34) was then subjected to the third stage of design: shortening of the guide length. FIG. 46A and FIG. 46B show engineering of the guide length. As shown in FIG. 46A, the effect of each guide length on ADAR1 and ADAR1+ADAR2 editing was determined. FIG. 46B shows the percent editing for the guide610 variants of varying length via ADAR1. As noted, the engineered guide RNA having a length of 92 nt with the mismatch positioned 60 nt from the end of the guide (0.92.60), displaying a LRRK2 editing of 60%, was selected as the top performing guide. TABLE 6 below recites the sequences of the engineered guide610 RNAs depicted in FIGS. 42A-46B. While the engineered guide RNA sequences in TABLE 6 are provided as DNA sequences with a T substituted for each U, the corresponding RNA sequences are also encompassed herein.

TABLE 6
Engineered LRRK2 Guide610 Variant Sequences

| | | | | | Left | Right |
|---|---|---|---|---|---|---|
| | | | | | Bar- | Bar- |
| SEQ | | | | | bell | bell |
| ID | | | | | Posi- | Posi- |
| NO | FIG. | Guide Name | Sequence | Structural Features (target/guide) | tion | tion |
| 4 | 42A | 610 (no loops) | CCCTGGTGTGCCCTCTGATGTTTTT | −49_1-1_wobble_U-G, −4_1- | | |
| | | | ATCCCCATTCTACATCTGTAGTGA | 1_mismatch_C-C, −3_1-1_wobble_U-G, | | |
| | | | GCAATTCCGTGCTCAGCAATCTTT | 0 1-1_mismatch_A-C, 2_1- | | |
| | | | GCAATGATGGCAGCATTGGGATA | 1_mismatch_C-U, 11 1-1_mismatch_G- | | |
| | | | CAGTGTGAAGAGCAGCA | G, 15_1-1_mismatch_U-U, 17_1- | | |
| | | | | 1_mismatch_C-U | | |
| 5 | 42B | 610 (−14, +26) | CCCTGGTGTGCCCTCTGATGTTTTT | −49 1-1_wobble_U-G, −14_6- | −14 | 26 |
| | | | TAGGGGATTCTACATCTGTAGTGA | 6_internal_loop-symmetric_UGCAAA- | | |
| | | | GCAATTCCGTGCTCAGCAATCAAA | AAACGU, −4_1-1_mismatch_C-C, | | |
| | | | CGTATGATGGCAGCATTGGGATAC | −3_1-1_wobble_U-G, 0_1- | | |
| | | | AGTGTGAAGAGCAGCA | 1_mismatch_A-C, 2_1-1_mismatch_C- | | |
| | | | | U, 11_1-1_mismatch_G-G, 15_1- | | |
| | | | | 1_mismatch_U-U, 17_1-1_mismatch_C- | | |
| | | | | U, 26_6-6_internal_loop- | | |
| | | | | symmetric_GGGGAU-UAGGGG | | |
| 204 | 43A−43C | 610 (−14, +26) | AACTTCAGGTGCACGAAACCCTG | −14_6-6_internal_loop- | −14 | 26 |
| | | 0.100.75 | GTGTGCCCTCTGATGTTTTTTAGG | symmetric_UGCAAA-AAACGU, −4_1- | | |
| | | | GGATTCTACATCTGTAGTGAGCAA | 1_mismatch_C-C, −3_1-1_wobble_U-G, | | |
| | | | TTCCGTGCTCAGCAATCAAACGTA | 0_1-1_mismatch_A-C, 2 1- | | |
| | | | TGATG | 1_mismatch_C-U, 11 1-1_mismatch_G- | | |
| | | | | G, 15_1-1_mismatch_U-U, 17_1- | | |
| | | | | 1_mismatch_C-U, 26_6- | | |
| | | | | 6_internal_loop-symmetric_GGGGAU- | | |
| | | | | UAGGGG | | |
| 205 | 43A−43C | 610 (−14, +26) | CAGGTGCACGAAACCCTGGTGTG | −14_6-6_internal_loop- | −14 | 26 |
| | | 0.100.70 | CCCTCTGATGTTTTTTAGGGGATT | symmetric_UGCAAA-AAACGU, −4_1- | | |
| | | | CTACATCTGTAGTGAGCAATTCCG | 1_mismatch_C-C, −3_1-1_wobble_U-G, | | |
| | | | TGCTCAGCAATCAAACGTATGATG | 0_1-1_mismatch_A-C, 2_1- | | |
| | | | GCAGC | 1_mismatch_C-U, 11_1-1_mismatch_G- | | |
| | | | | G, 15_1-1_mismatch_U-U, 17_1- | | |
| | | | | 1_mismatch_C-U, 26_6- | | |
| | | | | 6_internal_loop-symmetric_GGGGAU- | | |
| | | | | UAGGGG | | |
| 206 | 43A−43C | 610 (−14, +26) | GCACGAAACCCTGGTGTGCCCTCT | −14_6-6_internal_loop- | −14 | 26 |
| | | 0.100.65 | GATGTTTTTTAGGGGATTCTACAT | symmetric_UGCAAA-AAACGU, −4_1- | | |
| | | | CTGTAGTGAGCAATTCCGTGCTCA | 1_mismatch_C-C, −3_1-1_wobble_U-G, | | |
| | | | GCAATCAAACGTATGATGGCAGC | 0_1-1_mismatch_A-C, 2_1- | | |
| | | | ATTGG | 1_mismatch_C-U, 11_1-1_mismatch_G- | | |
| | | | | G, 15_1-1_mismatch_U-U, 17_1- | | |
| | | | | 1_mismatch_C-U, 26_6- | | |
| | | | | 6_internal_loop-symmetric_GGGGAU- | | |
| | | | | UAGGGG | | |
| 207 | 43A−43C | 610 (−14, +26) | AAACCCTGGTGTGCCCTCTGATGT | −14_6-6_internal_loop- | −14 | 26 |
| | | 0.100.60 | TTTTTAGGGGATTCTACATCTGTA | symmetric_UGCAAA-AAACGU, −4_1- | | |
| | | | GTGAGCAATTCCGTGCTCAGCAAT | 1_mismatch_C-C, −3_1-1_wobble_U-G, | | |
| | | | CAAACGTATGATGGCAGCATTGG | 0_1-1_mismatch_A-C, 2_1- | | |
| | | | GATAC | 1_mismatch_C-U, 11 1-1_mismatch_G- | | |
| | | | | G, 15_1-1_mismatch_U-U, 17_1- | | |
| | | | | 1_mismatch_C-U, 26_6- | | |
| | | | | 6_internal_loop-symmetric_GGGGAU- | | |
| | | | | UAGGGG | | |
| 208 | 43A−43C | 610 (−14, +26) | CTGGTGTGCCCTCTGATGTTTTTTA | −14_6-6_internal_loop- | −14 | 26 |
| | | 0.100.55 | GGGGATTCTACATCTGTAGTGAGC | symmetric_UGCAAA-AAACGU, −4_1- | | |
| | | | AATTCCGTGCTCAGCAATCAAACG | 1_mismatch_C-C, −3_1-1_wobble_U-G, | | |
| | | | TATGATGGCAGCATTGGGATACA | 0_1-1_mismatch_A-C, 2_1- | | |
| | | | GTGT | 1_mismatch_C-U, 11 1-1_mismatch_G- | | |
| | | | | G, 15_1-1_mismatch_U-U, 17_1- | | |
| | | | | 1_mismatch_C-U, 26_6- | | |
| | | | | 6_internal_loop-symmetric_GGGGAU- | | |
| | | | | UAGGGG | | |
| 209 | 43A−43C | 610 (−14, +26) | GTGCCCTCTGATGTTTTTTAGGGG | −14_6-6_internal_loop- | −14 | 26 |
| | | 0.100.50 | ATTCTACATCTGTAGTGAGCAATT | symmetric_UGCAAA-AAACGU, −4_1- | | |
| | | | CCGTGCTCAGCAATCAAACGTATG | 1_mismatch_C-C, −3_1-1_wobble_U-G, | | |
| | | | ATGGCAGCATTGGGATACAGTGT | 0_1-1_mismatch_A-C, 2_1- | | |
| | | | GAAAA | 1_mismatch_C-U, 11_1-1_mismatch_G- | | |
| | | | | G, 15_1-1_mismatch_U-U, 17_1- | | |
| | | | | 1_mismatch_C-U, 26_6- | | |
| | | | | 6_internal_loop-symmetric_GGGGAU- | | |
| | | | | UAGGGG | | |
| 5 | 43A−43C | 610 (−14, +26) | CCCTGGTGTGCCCTCTGATGTTTTT | −49_1-1_wobble_U-G, −14_6- | −14 | 26 |
| | | 0.113.57 | TAGGGGATTCTACATCTGTAGTGA | 6 internal_loop-symmetric_UGCAAA- | | |
| | | | GCAATTCCGTGCTCAGCAATCAAA | AAACGU, −4_1-1_mismatch_C-C, | | |
| | | | CGTATGATGGCAGCATTGGGATAC | −3_1-1_wobble_U-G, 0_1- | | |
| | | | AGTGTGAAGAGCAGCA | 1_mismatch_A-C, 2_1-1_mismatch_C- | | |
| | | | | U, 11_1-1_mismatch_G-G, 15_1- | | |
| | | | | 1_mismatch_U-U, 17_1-1_mismatch_C- | | |
| | | | | U, 26_6-6_internal_loop- | | |
| | | | | symmetric_GGGGAU-UAGGGG | | |
| 210 | 44A−44C | 610 (−14, +34) | AAACCCTGGTGTGCCCTCTGTCCA | −14_6-6_internal_loop- | −14 | 34 |
| | | 0.100.60 | AATTATCCCCATTCTACATCTGTA | symmetric_UGCAAA-AAACGU, −4_1- | | |
| | | | GTGAGCAATTCCGTGCTCAGCAAT | 1_mismatch_C-C, −3_1-1_wobble_U-G, | | |
| | | | CAAACGTATGATGGCAGCATTGG | 0_1-1_mismatch_A-C, 2 1- | | |
| | | | GATAC | 1_mismatch_C-U, 11_1-1_mismatch_G- | | |
| | | | | G, 15_1-1_mismatch_U-U, 17 1- | | |
| | | | | 1_mismatch_C-U, 34 6- | | |
| | | | | 6_internal_loop-symmetric_AAACAU- | | |
| | | | | UCCAAA | | |
| 211 | 44A−44C | 610 (−14, +32) | AAACCCTGGTGTGCCCTCTGATCA | −14_6-6_internal_loop- | −14 | 32 |
| | | 0.100.60 | AAACATCCCCATTCTACATCTGTA | symmetric_UGCAAA-AAACGU, −4_1- | | |
| | | | GTGAGCAATTCCGTGCTCAGCAAT | 1_mismatch_C-C, −3_1-1_wobble_U-G, | | |
| | | | CAAACGTATGATGGCAGCATTGG | 0_1-1_mismatch_A-C, 2_1- | | |
| | | | GATAC | 1_mismatch_C-U, 11_1-1_mismatch_G- | | |
| | | | | G, 15_1-1_mismatch_U-U, 17_1- | | |
| | | | | 1_mismatch_C-U, 32_6- | | |
| | | | | 6_internal_loop-symmetric_AAAAAC- | | |
| | | | | CAAAAC | | |
| 212 | 44A−44C | 610 (−14, +30) | AAACCCTGGTGTGCCCTCTGATGT | −14_6-6_internal_loop- | −14 | 30 |
| | | 0.100.60 | AAACTACCCCATTCTACATCTGTA | symmetric_UGCAAA-AAACGU, −4_1- | | |
| | | | GTGAGCAATTCCGTGCTCAGCAAT | 1_mismatch_C-C, −3_1-1_wobble_U-G, | | |
| | | | CAAACGTATGATGGCAGCATTGG | 0_1-1_mismatch_A-C, 2_1- | | |
| | | | GATAC | 1_mismatch_C-U, 11_1-1_mismatch_G- | | |
| | | | | G, 15_1-1_mismatch_U-U, 17_1- | | |
| | | | | 1_mismatch_C-U, 30_6- | | |
| | | | | 6_internal_loop-symmetric_AUAAAA- | | |
| | | | | AAACUA | | |
| 213 | 44A−44C | 610 (−14, +28) | AAACCCTGGTGTGCCCTCTGATGT | −14_6-6_internal_loop- | −14 | 28 |
| | | 0.100.60 | TTACTAGGCCATTCTACATCTGTA | symmetric_UGCAAA-AAACGU, −4_1- | | |
| | | | GTGAGCAATTCCGTGCTCAGCAAT | 1_mismatch_C-C, −3_1-1_wobble_U-G, | | |
| | | | CAAACGTATGATGGCAGCATTGG | 0 1-1_mismatch_A-C, 2 1- | | |
| | | | GATAC | 1_mismatch_C-U, 11_1-1_mismatch_G- | | |
| | | | | G, 15_1-1_mismatch_U-U, 17_1- | | |
| | | | | 1_mismatch_C-U, 28_6- | | |
| | | | | 6_internal_loop-symmetric_GGAUAA- | | |
| | | | | ACUAGG | | |
| 214 | 44A−44C | 610 (−14, +26) | AAACCCTGGTGTGCCCTCTGATGT | −14_6-6_internal_loop- | −14 | 26 |
| | | 0.100.60 | TTTTTAGGGGATTCTACATCTGTA | symmetric_UGCAAA-AAACGU, −4_1- | | |
| | | | GTGAGCAATTCCGTGCTCAGCAAT | 1_mismatch_C-C, −3_1-1_wobble_U-G, | | |
| | | | CAAACGTATGATGGCAGCATTGG | 0_1-1_mismatch_A-C, 2_1- | | |
| | | | GATAC | 1_mismatch_C-U, 11_1-1_mismatch_G- | | |
| | | | | G, 15_1-1_mismatch_U-U, 17_1- | | |
| | | | | 1_mismatch_C-U, 26_6- | | |
| | | | | 6_internal_loop-symmetric_GGGGAU- | | |
| | | | | UAGGGG | | |
| 215 | 44A−44C | 610 (−14, +25) | AAACCCTGGTGTGCCCTCTGATGT | −14_6-6_internal_loop- | −14 | 28 |
| | | 0.100.60 | TTTTAAGGGGTTTCTACATCTGTA | symmetric_UGCAAA-AAACGU, −4_1- | | |
| | | | GTGAGCAATTCCGTGCTCAGCAAT | 1_mismatch_C-C, −3_1-1_wobble_U-G, | | |
| | | | CAAACGTATGATGGCAGCATTGG | 0_1-1_mismatch_A-C, 2_1- | | |
| | | | GATAC | 1_mismatch_C-U, 11_1-1_mismatch_G- | | |
| | | | | G, 15_1-1_mismatch_U-U, 17_1- | | |
| | | | | 1_mismatch_C-U, 28_6- | | |
| | | | | 6_internal_loop-symmetric_GGAUAA- | | |
| | | | | ACUAGG | | |
| 216 | 44A−44C | 610 (−14, +24) | AAACCCTGGTGTGCCCTCTGATGT | −14_6-6_internal_loop- | −14 | 24 |
| | | 0.100.60 | TTTTATGGGGTCTCTACATCTGTA | symmetric_UGCAAA-AAACGU, −4_1- | | |
| | | | GTGAGCAATTCCGTGCTCAGCAAT | 1_mismatch_C-C, −3_1-1_wobble_U-G, | | |
| | | | CAAACGTATGATGGCAGCATTGG | 0_1-1_mismatch_A-C, 2 1- | | |
| | | | GATAC | 1_mismatch_C-U, 11 1-1_mismatch_G- | | |
| | | | | G, 15_1-1_mismatch_U-U, 17_1- | | |
| | | | | 1_mismatch_C-U, 24_6- | | |
| | | | | 6_internal_loop-symmetric_AUGGGG- | | |
| | | | | GGGGUC | | |
| 217 | 44A−44C | 610 (−14, +23) | AAACCCTGGTGTGCCCTCTGATGT | −14_6-6_internal_loop- | −14 | 23 |
| | | 0.100.60 | TTTTATCGGGTCACTACATCTGTA | symmetric_UGCAAA-AAACGU, −4_1- | | |
| | | | GTGAGCAATTCCGTGCTCAGCAAT | 1_mismatch_C-C, −3_1-1_wobble_U-G, | | |
| | | | CAAACGTATGATGGCAGCATTGG | 0_1-1_mismatch_A-C, 2 1- | | |
| | | | GATAC | 1_mismatch_C-U, 11_1-1_mismatch_G- | | |
| | | | | G, 15_1-1_mismatch_U-U, 17_1- | | |
| | | | | 1_mismatch_C-U, 23_6- | | |
| | | | | 6_internal_loop-symmetric_AAUGGG- | | |
| | | | | GGGUCA | | |
| 218 | 44A−44C | 610 (−14, +22) | AAACCCTGGTGTGCCCTCTGATGT | −14_6-6_internal_loop- | −14 | 22 |
| | | 0.100.60 | TTTTATCCGGTCAGTACATCTGTA | symmetric_UGCAAA-AAACGU, −4_1- | | |
| | | | GTGAGCAATTCCGTGCTCAGCAAT | 1_mismatch_C-C, −3_1-1_wobble_U-G, | | |
| | | | CAAACGTATGATGGCAGCATTGG | 0_1-1_mismatch_A-C, 2_1- | | |
| | | | GATAC | 1_mismatch_C-U, 11_1-1_mismatch_G- | | |
| | | | | G, 15_1-1_mismatch_U-U, 17_1- | | |
| | | | | 1_mismatch_C-U, 22_6- | | |
| | | | | 6_internal_loop-symmetric_GAAUGG- | | |
| | | | | GGUCAG | | |
| 219 | 45A−45B | 610 (−24, +34) | AAACCCTGGTGTGCCCTCTGTCCA | −24 6-6_internal_loop- | −24 | 34 |
| | | 0.100.60 | AATTATCCCCATTCTACATCTGTA | symmetric_CUGCCA-ACUGUC, −4_1- | | |
| | | | GTGAGCAATTCCGTGCTCAGCAAT | 1_mismatch_C-C, −3_1-1_wobble_U-G, | | |
| | | | CTTTGCAATGAACTGTCCATTGGG | 0_1-1_mismatch_A-C, 2_1- | | |
| | | | ATAC | 1_mismatch_C-U, 11_1-1_mismatch_G- | | |
| | | | | G, 15_1-1_mismatch_U-U, 17_1- | | |
| | | | | 1_mismatch_C-U, 34 6- | | |
| | | | | 6_internal_loop-symmetric_AAACAU- | | |
| | | | | UCCAAA | | |
| 220 | 45A−45B | 610 (−22, +34) | AAACCCTGGTGTGCCCTCTGTCCA | −22_6-6_internal_loop- | −22 | 34 |
| | | 0.100.60 | AATTATCCCCATTCTACATCTGTA | symmetric_GCCAUC-CUACUG, −4_1- | | |
| | | | GTGAGCAATTCCGTGCTCAGCAAT | 1_mismatch_C-C, −3_1-1_wobble_U-G, | | |
| | | | CTTTGCAATCTACTGAGCATTGGG | 0_1-1_mismatch_A-C, 2_1- | | |
| | | | ATAC | 1_mismatch_C-U, 11_1-1_mismatch_G- | | |
| | | | | G, 15_1-1_mismatch_U-U, 17_1- | | |
| | | | | 1_mismatch_C-U, 34_6- | | |
| | | | | 6_internal_loop-symmetric_AAACAU- | | |
| | | | | UCCAAA | | |
| 221 | 45A−45B | 610 (−20, +34) | AAACCCTGGTGTGCCCTCTGTCCA | −20_6-6_internal_loop- | −20 | 34 |
| | | 0.100.60 | AATTATCCCCATTCTACATCTGTA | symmetric_CAUCAU-CCCUAC, −4 1- | | |
| | | | GTGAGCAATTCCGTGCTCAGCAAT | 1_mismatch_C-C, −3_1-1_wobble_U-G, | | |
| | | | CTTTGCACCCTACGCAGCATTGGG | 0_1-1_mismatch_A-C, 2_1- | | |
| | | | ATAC | 1_mismatch_C-U, 11_1-1_mismatch_G- | | |
| | | | | G, 15_1-1_mismatch_U-U, 17_1- | | |
| | | | | 1_mismatch_C-U, 34_6- | | |
| | | | | 6_internal_loop-symmetric_AAACAU- | | |
| | | | | UCCAAA | | |
| 222 | 45A−45B | 610 (−18, +34) | AAACCCTGGTGTGCCCTCTGTCCA | −18_6-6_internal_loop- | −18 | 34 |
| | | 0.100.60 | AATTATCCCCATTCTACATCTGTA | symmetric_UCAUUG-GUCCCU, −4_1- | | |
| | | | GTGAGCAATTCCGTGCTCAGCAAT | 1_mismatch_C-C, −3_1-1_wobble_U-G, | | |
| | | | CTTTGGTCCCTTGGCAGCATTGGG | 0_1-1_mismatch_A-C, 2_1- | | |
| | | | ATAC | 1_mismatch_C-U, 11 1-1_mismatch_G- | | |
| | | | | G, 15_1-1_mismatch_U-U, 17 1- | | |
| | | | | 1_mismatch_C-U, 34 6- | | |
| | | | | 6_internal_loop-symmetric_AAACAU- | | |
| | | | | UCCAAA | | |
| 223 | 45A−45B | 610 (−16, +34) | AAACCCTGGTGTGCCCTCTGTCCA | −16_6-6_internal_loop- | −16 | 34 |
| | | 0.100.60 | AATTATCCCCATTCTACATCTGTA | symmetric_AUUGCA-ACGUCC, −4_1- | | |
| | | | GTGAGCAATTCCGTGCTCAGCAAT | 1_mismatch_C-C, −3_1-1_wobble_U-G, | | |
| | | | CTTACGTCCGATGGCAGCATTGGG | 0_1-1_mismatch_A-C, 2_1- | | |
| | | | ATAC | 1_mismatch_C-U, 11_1-1_mismatch_G- | | |
| | | | | G, 15_1-1_mismatch_U-U, 17_1- | | |
| | | | | 1_mismatch_C-U, 34_6- | | |
| | | | | 6_internal_loop-symmetric_AAACAU- | | |
| | | | | UCCAAA | | |
| 224 | 45A−45B | 610 (−14, +34) | AAACCCTGGTGTGCCCTCTGTCCA | −14_6-6_internal_loop- | −14 | 34 |
| | | 0.100.60 | AATTATCCCCATTCTACATCTGTA | symmetric_UGCAAA-AAACGU, −4 1- | | |
| | | | GTGAGCAATTCCGTGCTCAGCAAT | 1_mismatch_C-C, −3_1-1_wobble_U-G, | | |
| | | | CAAACGTATGATGGCAGCATTGG | 0_1-1_mismatch_A-C, 2_1- | | |
| | | | GATAC | 1_mismatch_C-U, 11_1-1_mismatch_G- | | |
| | | | | G, 15_1-1_mismatch_U-U, 17_1- | | |
| | | | | 1_mismatch_C-U, 34_6- | | |
| | | | | 6_internal_loop-symmetric_AAACAU- | | |
| | | | | UCCAAA | | |
| 225 | 45A−45B | 610 (-12, +34) | AAACCCTGGTGTGCCCTCTGTCCA | -12_6-6_internal_loop- | -12 | 34 |
| | | 0.100.60 | AATTATCCCCATTCTACATCTGTA | symmetric_CAAAGA-AGAAAC, −4_1- | | |
| | | | GTGAGCAATTCCGTGCTCAGCAAA | 1_mismatch_C-C, −3_1-1_wobble_U-G, | | |
| | | | GAAACCAATGATGGCAGCATTGG | 0_1-1_mismatch_A-C, 2_1- | | |
| | | | GATAC | 1_mismatch_C-U, 11_1-1_mismatch_G- | | |
| | | | | G, 15_1-1_mismatch_U-U, 17_1- | | |
| | | | | 1_mismatch_C-U, 34_6- | | |
| | | | | 6_internal_loop-symmetric_AAACAU- | | |
| | | | | UCCAAA | | |
| 226 | 45A−45B | 610 (−10, +34) | AAACCCTGGTGTGCCCTCTGTCCA | −10_6-6_internal_loop- | −10 | 34 |
| | | 0.100.60 | AATTATCCCCATTCTACATCTGTA | symmetric_AAGAUU-UCAGAA, −4_1- | | |
| | | | GTGAGCAATTCCGTGCTCAGCTCA | 1_mismatch_C-C, −3_1-1_wobble_U-G, | | |
| | | | GAATGCAATGATGGCAGCATTGG | 0_1-1_mismatch_A-C, 2_1- | | |
| | | | GATAC | 1_mismatch_C-U, 11_1-1_mismatch_G- | | |
| | | | | G, 15_1-1_mismatch_U-U, 17_1- | | |
| | | | | 1_mismatch_C-U, 34_6- | | |
| | | | | 6_internal_loop-symmetric_AAACAU- | | |
| | | | | UCCAAA | | |
| 227 | 46A−46B | 610 (−10, +34) | AAACCCTGGTGTGCCCTCTGTCCA | −10_6-6_internal_loop- | −10 | 34 |
| | | 0.90.60 | AATTATCCCCATTCTACATCTGTA | symmetric_AAGAUU-UCAGAA, −4_1- | | |
| | | | GTGAGCAATTCCGTGCTCAGCTCA | 1_mismatch_C-C, −3_1-1_wobble_U-G, | | |
| | | | GAATGCAATGATGGCAGC | 0_1-1_mismatch_A-C, 2_1- | | |
| | | | | 1_mismatch_C-U, 11_1-1_mismatch_G- | | |
| | | | | G, 15_1-1_mismatch_U-U, 17_1- | | |
| | | | | 1_mismatch_C-U, 34_6- | | |
| | | | | 6_internal_loop-symmetric_AAACAU- | | |
| | | | | UCCAAA | | |
| 228 | 46A−46B | 610 (−10, +34) | AAACCCTGGTGTGCCCTCTGTCCA | −10_6-6_internal_loop- | −10 | 34 |
| | | 0.92.60 | AATTATCCCCATTCTACATCTGTA | symmetric_AAGAUU-UCAGAA, −4_1- | | |
| | | | GTGAGCAATTCCGTGCTCAGCTCA | 1_mismatch_C-C, −3_1-1_wobble_U-G, | | |
| | | | GAATGCAATGATGGCAGCAT | 0_1-1_mismatch_A-C, 2_1- | | |
| | | | | 1_mismatch_C-U, 11_1-1_mismatch_G- | | |
| | | | | G, 15_1-1_mismatch_U-U, 17_1- | | |
| | | | | 1_mismatch_C-U, 34_6- | | |
| | | | | 6_internal_loop-symmetric_AAACAU- | | |
| | | | | UCCAAA | | |
| 229 | 46A−46B | 610 (−10, +34) | AAACCCTGGTGTGCCCTCTGTCCA | −10_6-6_internal_loop- | −10 | 34 |
| | | 0.94.60 | AATTATCCCCATTCTACATCTGTA | symmetric_AAGAUU-UCAGAA, −4_1- | | |
| | | | GTGAGCAATTCCGTGCTCAGCTCA | 1_mismatch_C-C, −3_1-1_wobble_U-G, | | |
| | | | GAATGCAATGATGGCAGCATTG | 0_1-1_mismatch_A-C, 2_1- | | |
| | | | | 1_mismatch_C-U, 11_1-1_mismatch_G- | | |
| | | | | G, 15_1-1_mismatch_U-U, 17_1- | | |
| | | | | 1_mismatch_C-U, 34_6- | | |
| | | | | 6_internal_loop-symmetric_AAACAU- | | |
| | | | | UCCAAA | | |
| 230 | 46A−46B | 610 (−10, +34) | AAACCCTGGTGTGCCCTCTGTCCA | −10_6-6_internal_loop- | −10 | 34 |
| | | 0.96.60 | AATTATCCCCATTCTACATCTGTA | symmetric_AAGAUU-UCAGAA, −4 1- | | |
| | | | GTGAGCAATTCCGTGCTCAGCTCA | 1_mismatch_C-C, −3_1-1_wobble_U-G, | | |
| | | | GAATGCAATGATGGCAGCATTGG | 0_1-1_mismatch_A-C, 2_1- | | |
| | | | G | 1_mismatch_C-U, 11_1-1_mismatch_G- | | |
| | | | | G, 15_1-1_mismatch_U-U, 17_1- | | |
| | | | | 1_mismatch_C-U, 34_6- | | |
| | | | | 6_internal_loop-symmetric_AAACAU- | | |
| | | | | UCCAAA | | |
| 231 | 46A−46B | 610 (−10, +34) | AAACCCTGGTGTGCCCTCTGTCCA | −10_6-6_internal_loop- | −10 | 34 |
| | | 0.98.60 | AATTATCCCCATTCTACATCTGTA | symmetric_AAGAUU-UCAGAA, −4 1- | | |
| | | | GTGAGCAATTCCGTGCTCAGCTCA | 1_mismatch_C-C, −3_1-1_wobble_U-G, | | |
| | | | GAATGCAATGATGGCAGCATTGG | 0_1-1_mismatch_A-C, 2 1- | | |
| | | | GAT | 1_mismatch_C-U, 11_1-1_mismatch_G- | | |
| | | | | G, 15_1-1_mismatch_U-U, 17_1- | | |
| | | | | 1_mismatch_C-U, 34_6- | | |
| | | | | 6_internal_loop-symmetric_AAACAU- | | |
| | | | | UCCAAA | | |
| 232 | 46A−46B | 610 (−10, +34) | GTGCCCTCTGTCCAAATTATCCCC | −10_6-6_internal_loop- | −10 | 34 |
| | | 0.90.50 | ATTCTACATCTGTAGTGAGCAATT | symmetric_AAGAUU-UCAGAA, −4 1- | | |
| | | | CCGTGCTCAGCTCAGAATGCAATG | 1_mismatch_C-C, −3_1-1_wobble_U-G, | | |
| | | | ATGGCAGCATTGGGATAC | 0_1-1_mismatch_A-C, 2_1- | | |
| | | | | 1_mismatch_C-U, 11_1-1_mismatch_G- | | |
| | | | | G, 15_1-1_mismatch_U-U, 17_1- | | |
| | | | | 1_mismatch_C-U, 34_6- | | |
| | | | | 6_internal_loop-symmetric_AAACAU- | | |
| | | | | UCCAAA | | |
| 233 | 46A−46B | 610 (−10, +34) | GTGTGCCCTCTGTCCAAATTATCC | −10_6-6_internal_loop- | −10 | 34 |
| | | 0.92.52 | CCATTCTACATCTGTAGTGAGCAA | symmetric_AAGAUU-UCAGAA, −4 1- | | |
| | | | TTCCGTGCTCAGCTCAGAATGCAA | 1_mismatch_C-C, −3_1-1_wobble_U-G, | | |
| | | | TGATGGCAGCATTGGGATAC | 0_1-1_mismatch_A-C, 2_1- | | |
| | | | | 1_mismatch_C-U, 11_1-1_mismatch_G- | | |
| | | | | G, 15_1-1_mismatch_U-U, 17_1- | | |
| | | | | 1_mismatch_C-U, 34_6- | | |
| | | | | 6_internal_loop-symmetric_AAACAU- | | |
| | | | | UCCAAA | | |
| 234 | 46A−46B | 610 (−10, +34) | TGGTGTGCCCTCTGTCCAAATTAT | −10_6-6_internal_loop- | −10 | 34 |
| | | 0.94.54 | CCCCATTCTACATCTGTAGTGAGC | symmetric_AAGAUU-UCAGAA, −4 1- | | |
| | | | AATTCCGTGCTCAGCTCAGAATGC | 1_mismatch_C-C, −3_1-1_wobble_U-G, | | |
| | | | AATGATGGCAGCATTGGGATAC | 0 1-1_mismatch_A-C, 2_1- | | |
| | | | | 1_mismatch_C-U, 11_1-1_mismatch_G- | | |
| | | | | G, 15_1-1_mismatch_U-U, 17_1- | | |
| | | | | 1_mismatch_C-U, 34_6- | | |
| | | | | 6_internal_loop-symmetric_AAACAU- | | |
| | | | | UCCAAA | | |
| 235 | 46A−46B | 610 (−10, +34) | CCTGGTGTGCCCTCTGTCCAAATT | −10_6-6_internal_loop- | −10 | 34 |
| | | 0.96.56 | ATCCCCATTCTACATCTGTAGTGA | symmetric_AAGAUU-UCAGAA, −4 1- | | |
| | | | GCAATTCCGTGCTCAGCTCAGAAT | 1_mismatch_C-C, −3_1-1_wobble_U-G, | | |
| | | | GCAATGATGGCAGCATTGGGATA | 0_1-1_mismatch_A-C, 2_1- | | |
| | | | C | 1_mismatch_C-U, 11_1-1_mismatch_G- | | |
| | | | | G, 15_1-1_mismatch_U-U, 17_1- | | |
| | | | | 1_mismatch_C-U, 34 6- | | |
| | | | | 6_internal_loop-symmetric_AAACAU- | | |
| | | | | UCCAAA | | |
| 236 | 46A−46B | 610 (−10, +34) | ACCCTGGTGTGCCCTCTGTCCAAA | −10_6-6_internal_loop- | −10 | 34 |
| | | 0.98.58 | TTATCCCCATTCTACATCTGTAGT | symmetric_AAGAUU-UCAGAA, −4 1- | | |
| | | | GAGCAATTCCGTGCTCAGCTCAGA | 1_mismatch_C-C, −3_1-1_wobble_U-G, | | |
| | | | ATGCAATGATGGCAGCATTGGGA | 0_1-1_mismatch_A-C, 2_1- | | |
| | | | TAC | 1_mismatch_C-U, 11_1-1_mismatch_G- | | |
| | | | | G, 15_1-1_mismatch_U-U, 17_1- | | |
| | | | | 1_mismatch_C-U, 34_6- | | |
| | | | | 6_internal_loop-symmetric_AAACAU- | | |
| | | | | UCCAAA | | |
| 237 | 46A−46B | 610 (−10, +34) | AAACCCTGGTGTGCCCTCTGTCCA | −10_6-6_internal_loop- | −10 | 34 |
| | | 0.100.60 | AATTATCCCCATTCTACATCTGTA | symmetric_AAGAUU-UCAGAA, −4_1- | | |
| | | | GTGAGCAATTCCGTGCTCAGCTCA | 1_mismatch_C-C, −3_1-1_wobble_U-G, | | |
| | | | GAATGCAATGATGGCAGCATTGG | 0_1-1_mismatch_A-C, 2_1- | | |
| | | | GATAC | 1_mismatch_C-U, 11_1-1_mismatch_G- | | |
| | | | | G, 15_1-1_mismatch_U-U, 17_1- | | |
| | | | | 1_mismatch_C-U, 34_6- | | |
| | | | | 6_internal_loop-symmetric_AAACAU- | | |
| | | | | UCCAAA | | |

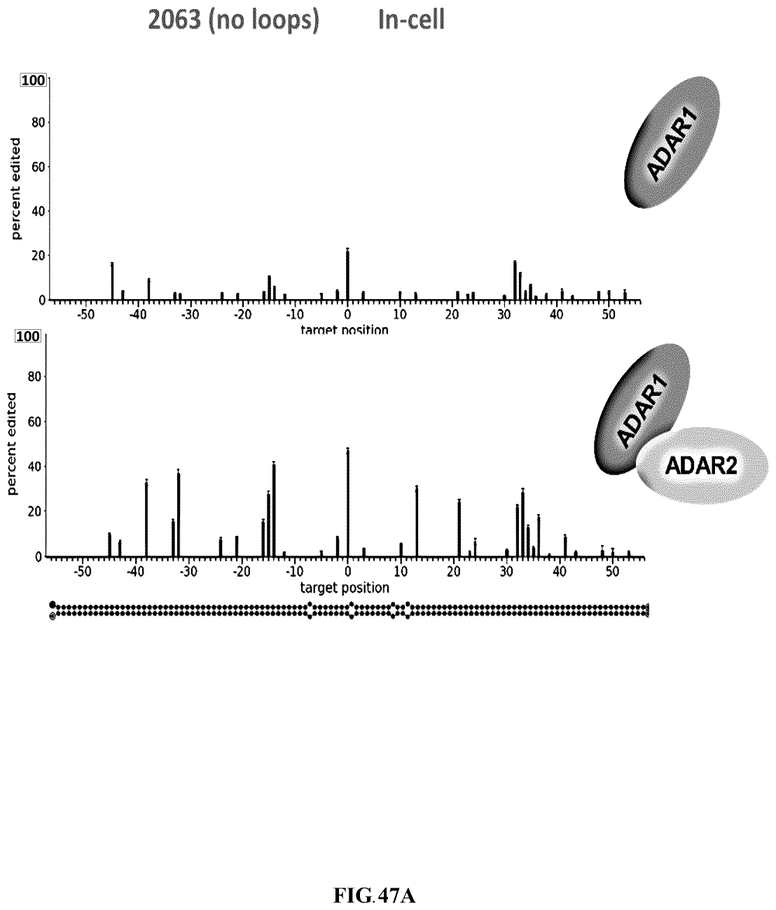

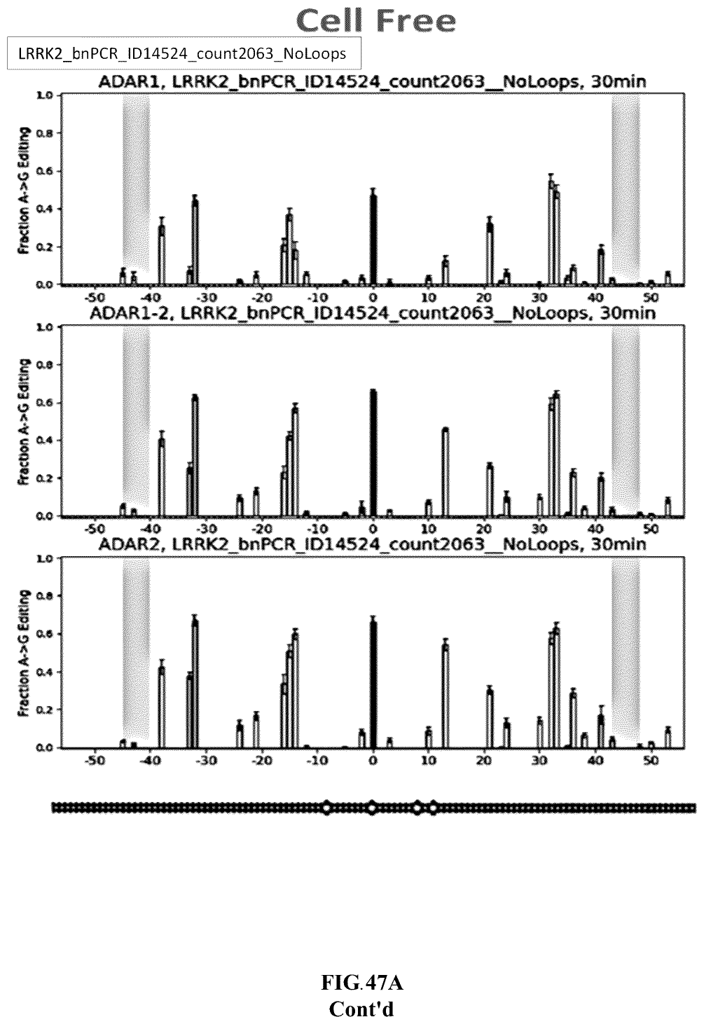

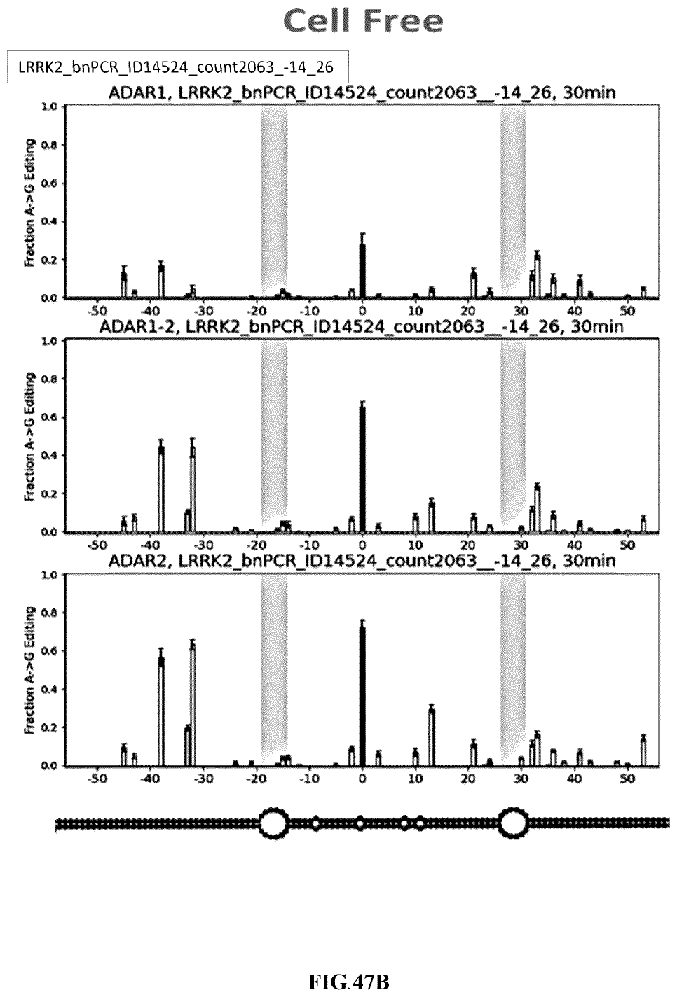

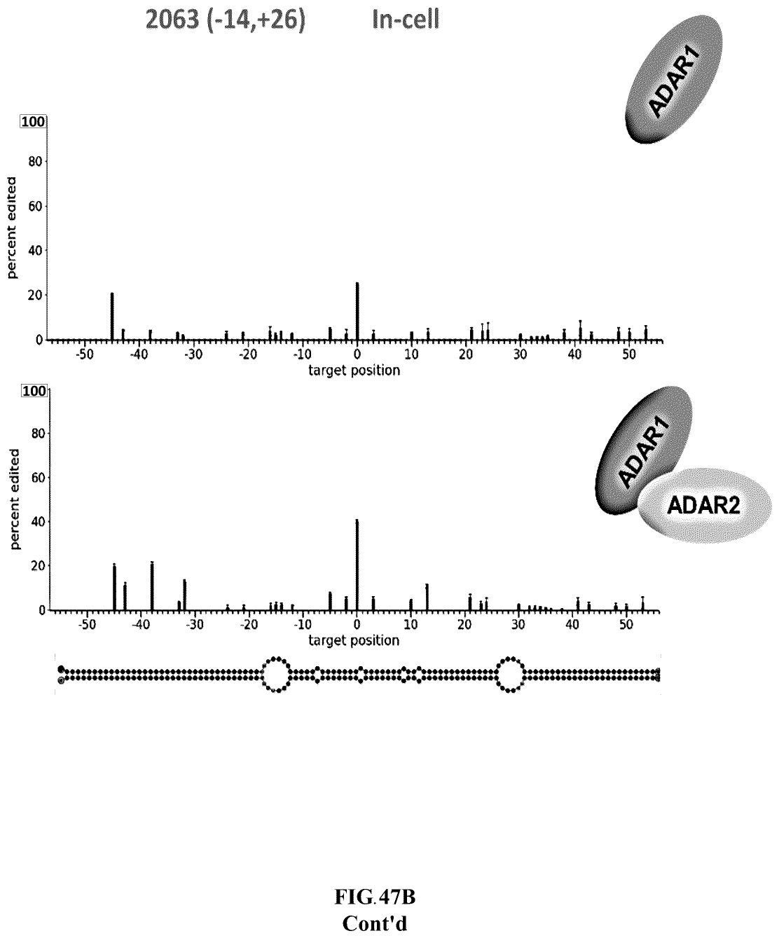

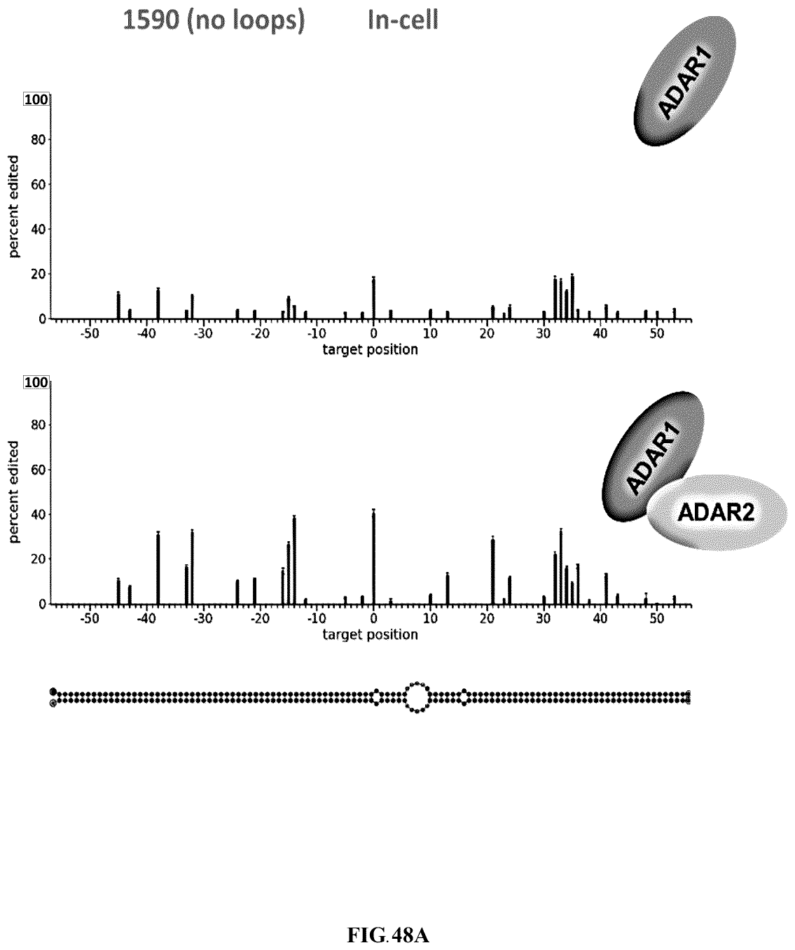

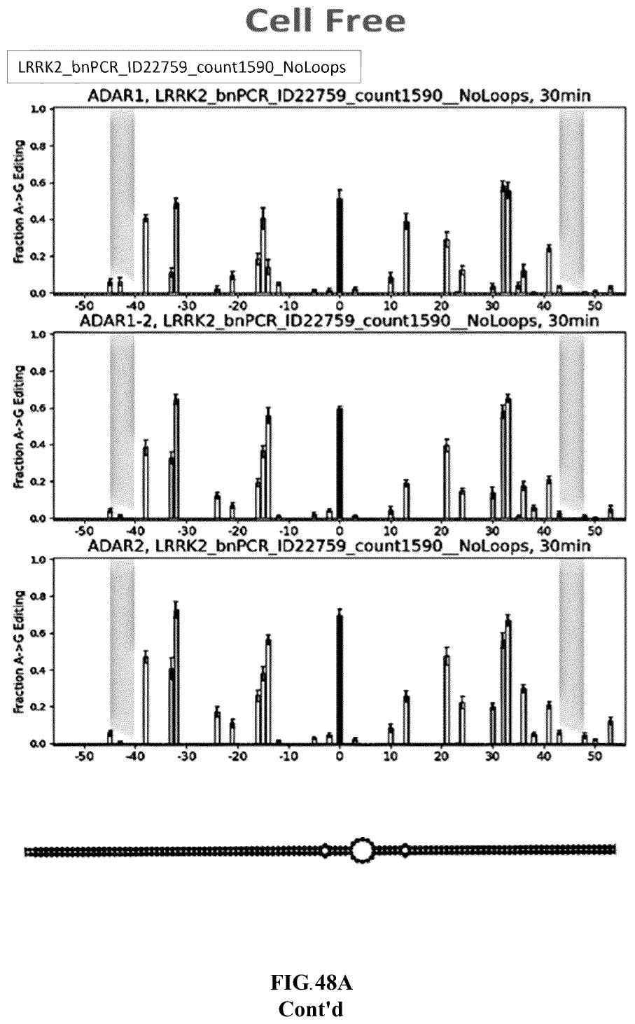

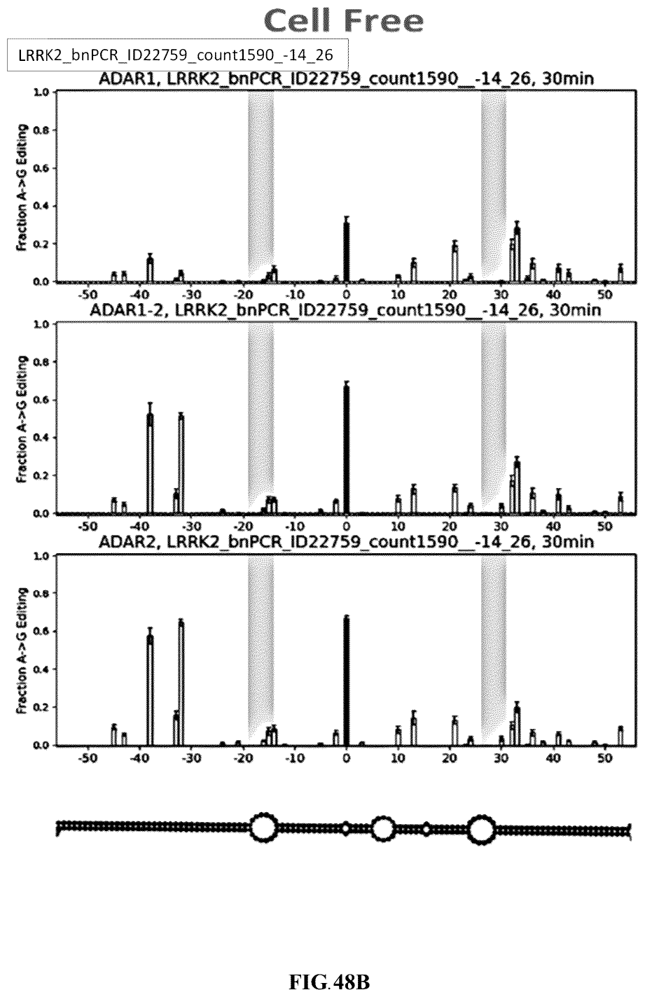

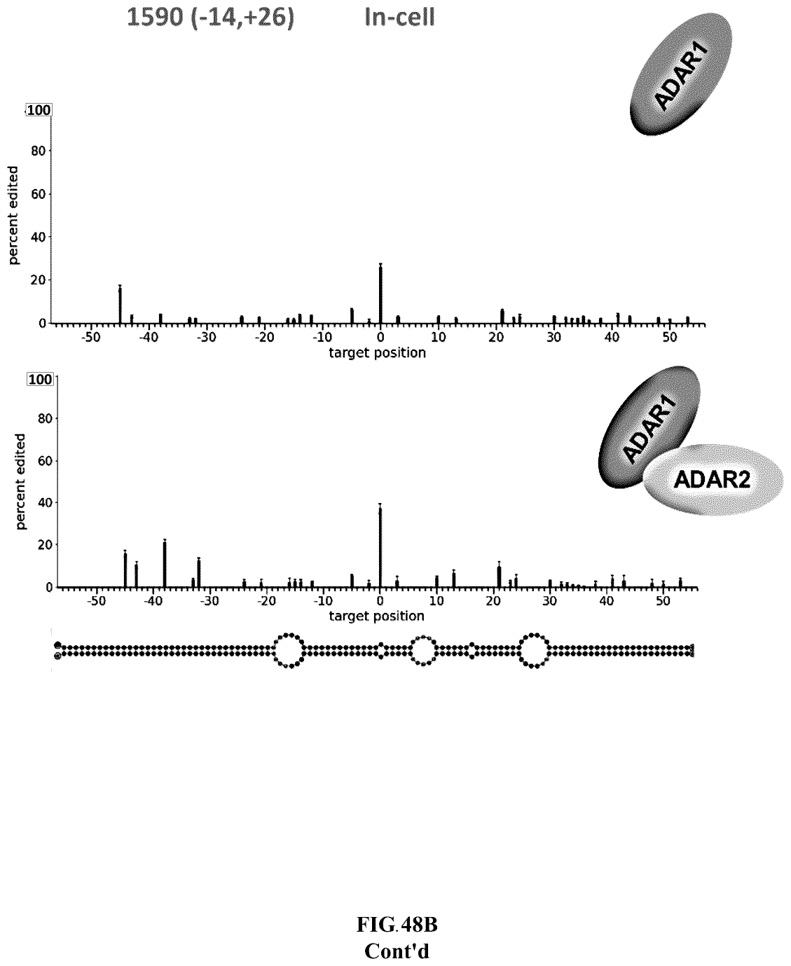

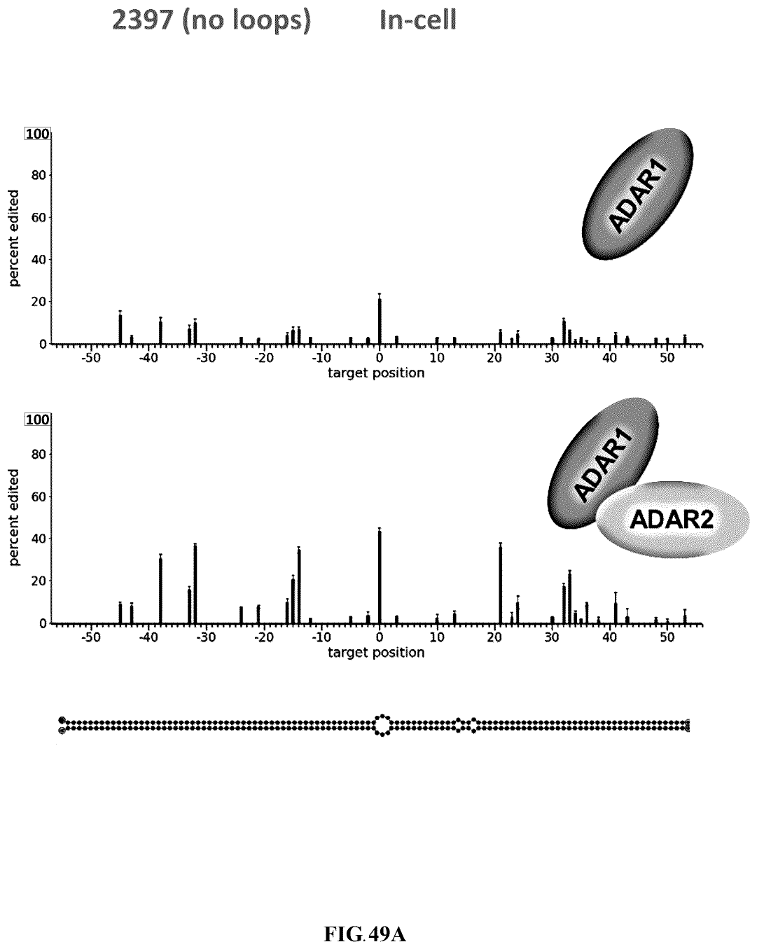

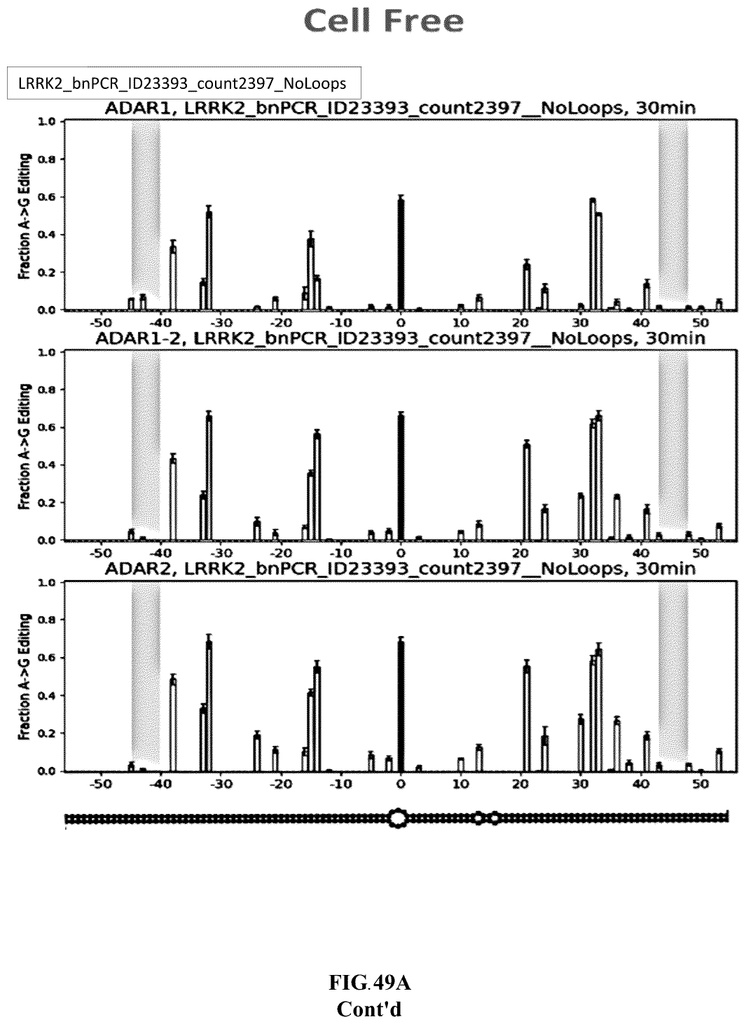

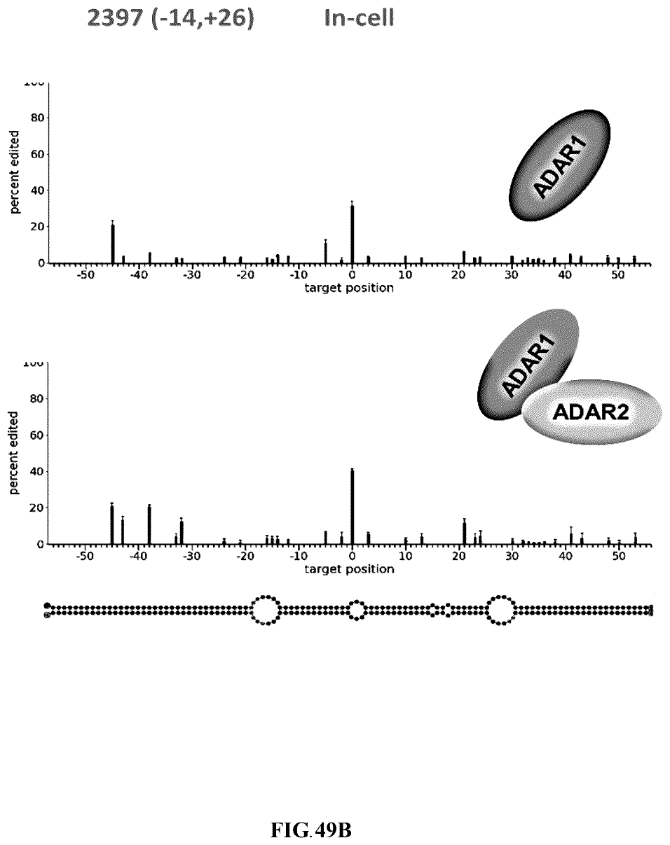

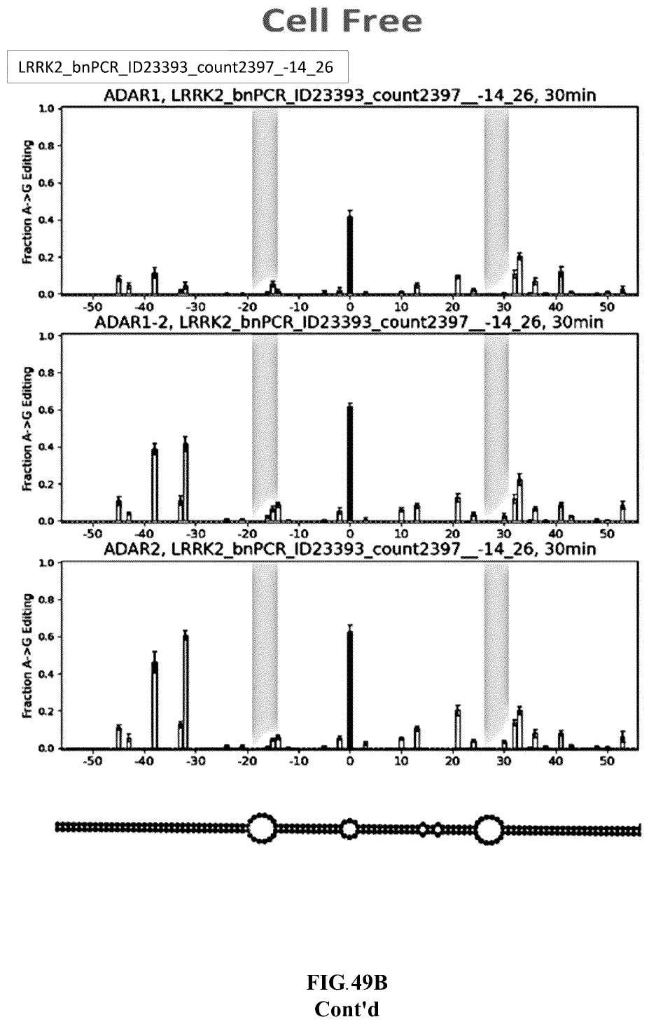

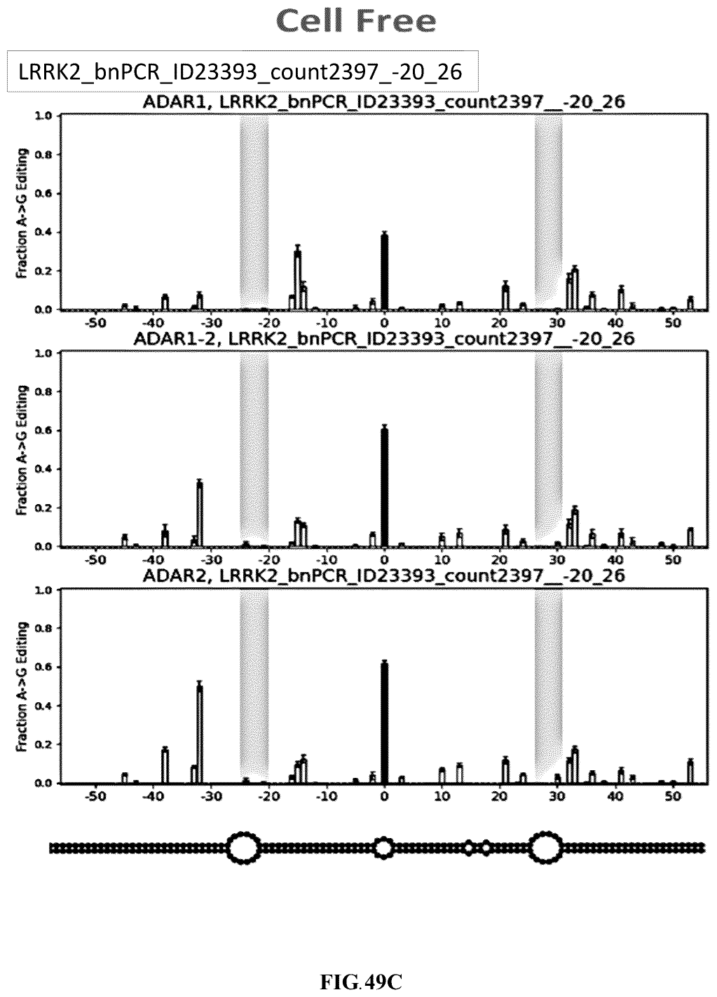

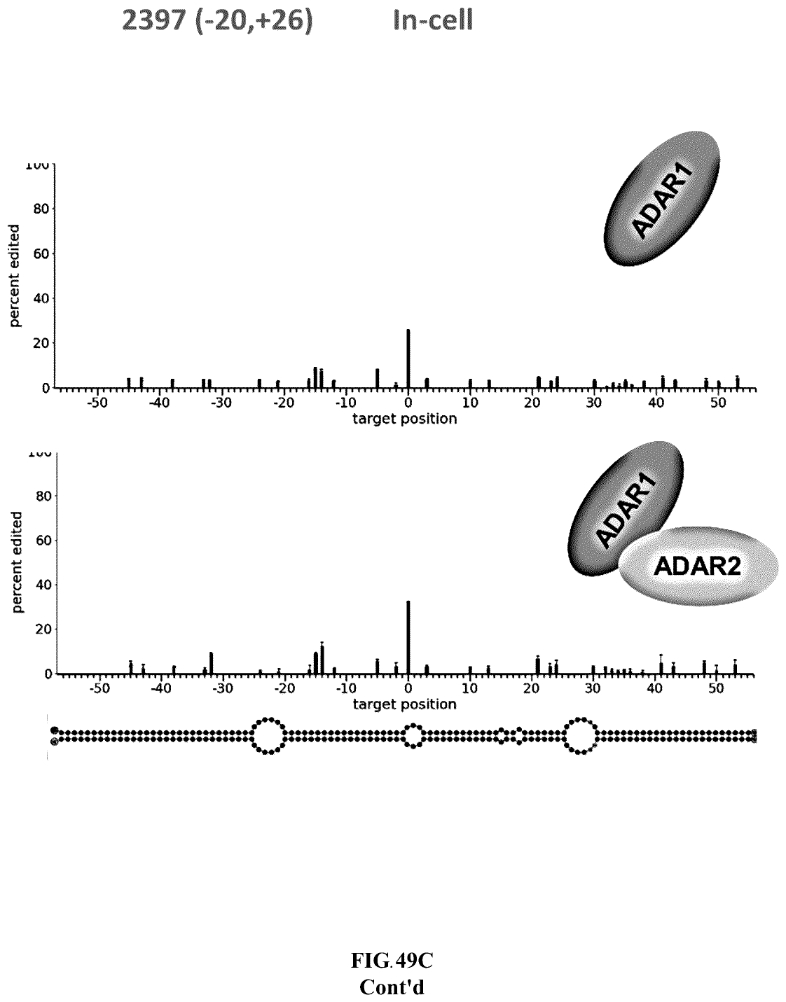

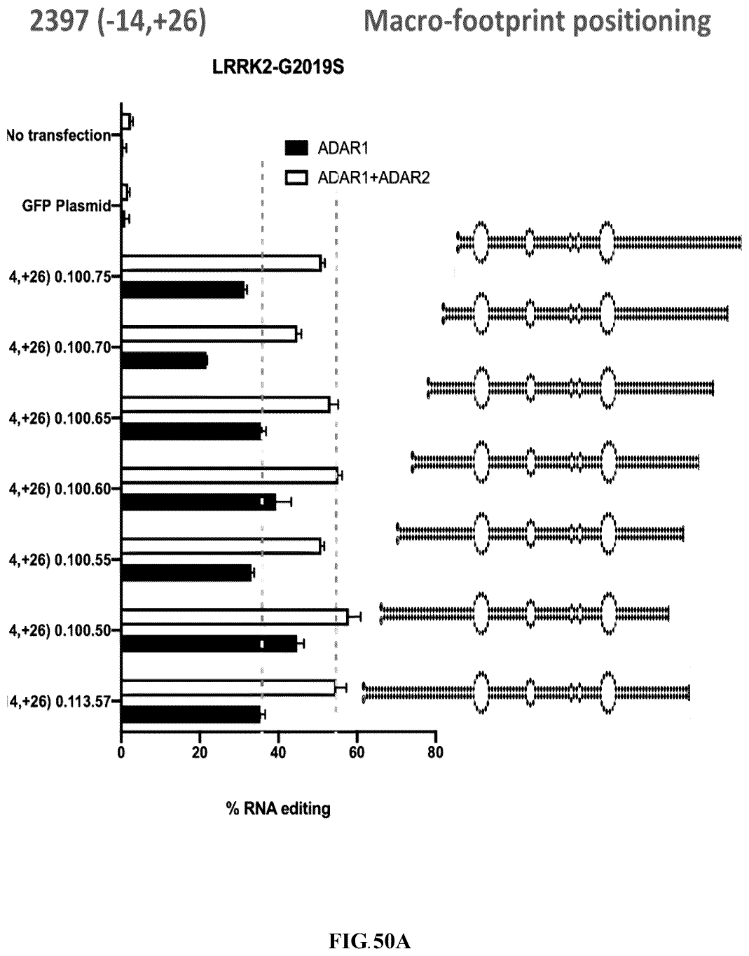

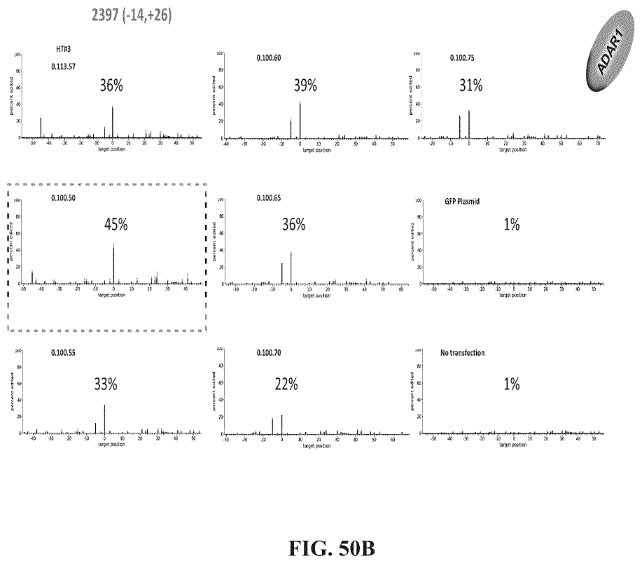

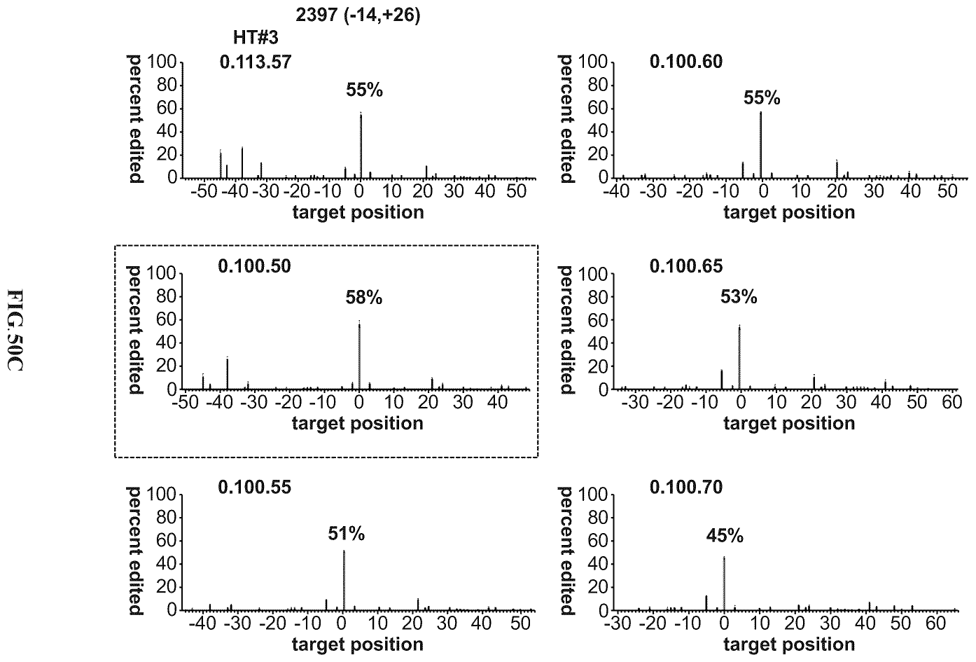

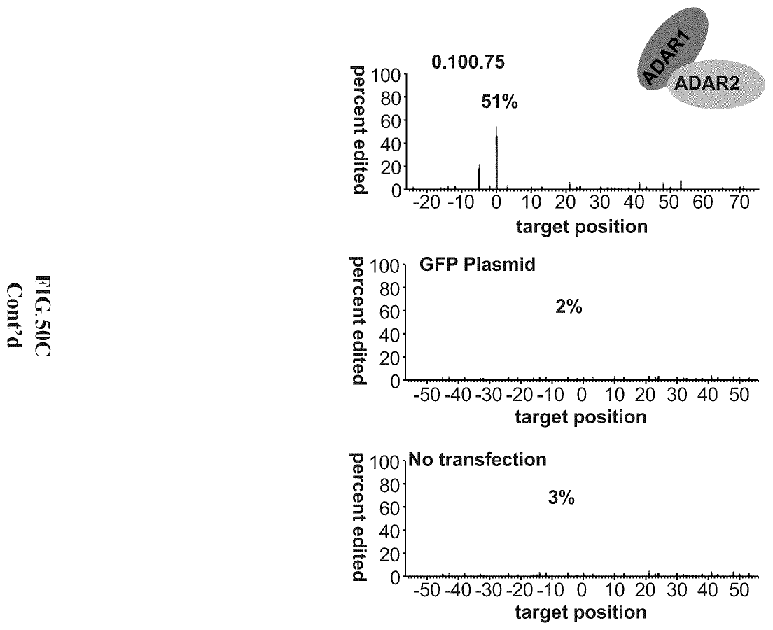

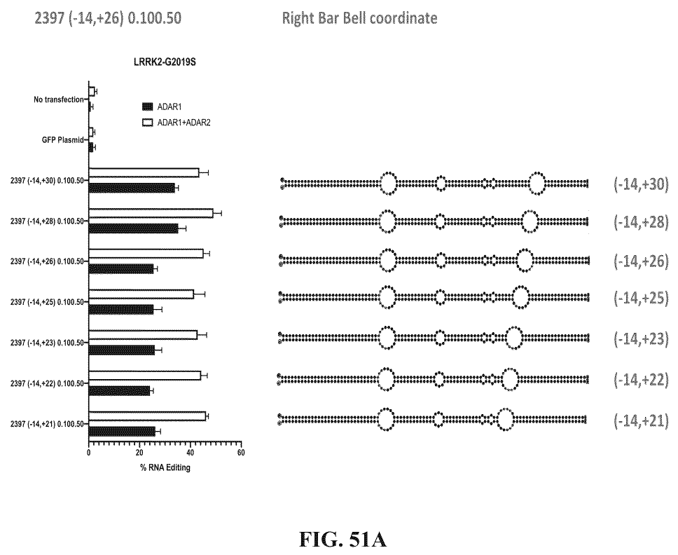

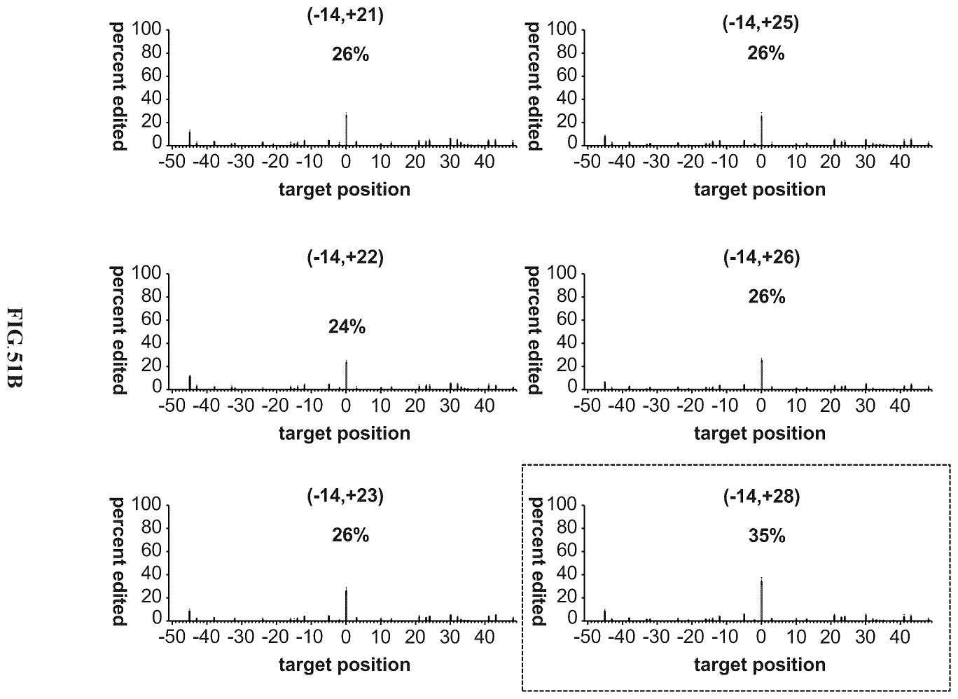

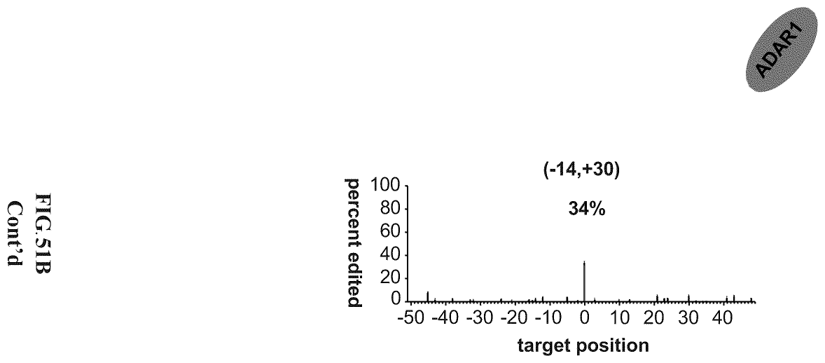

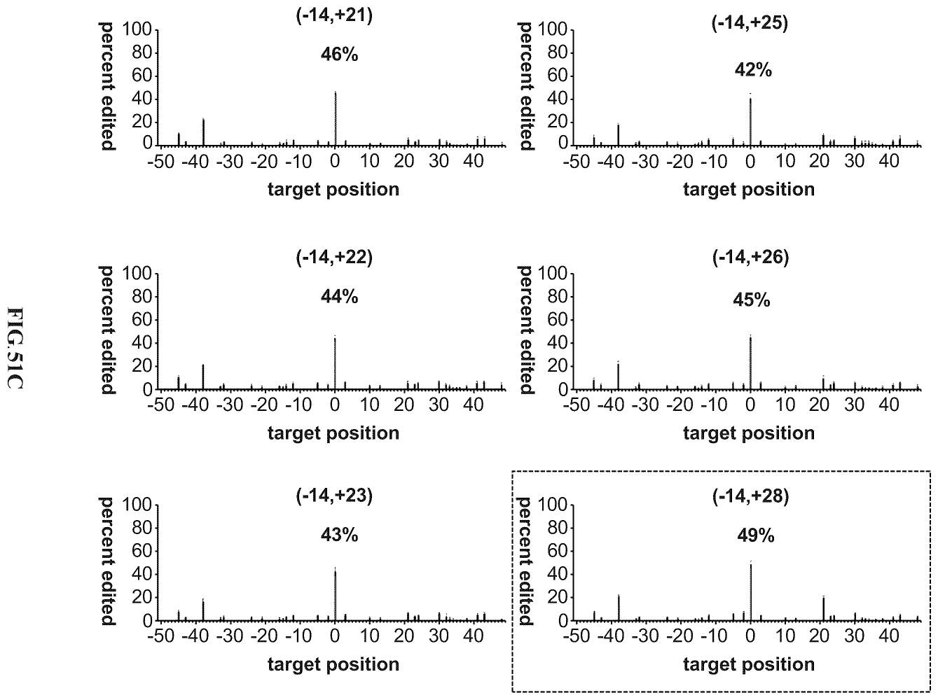

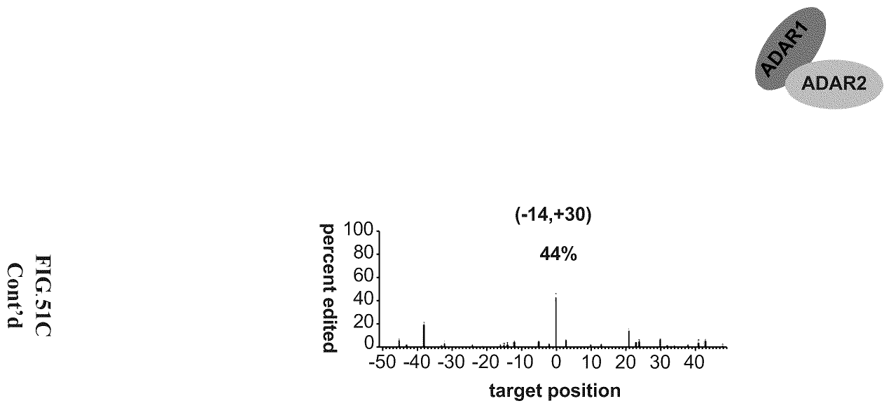

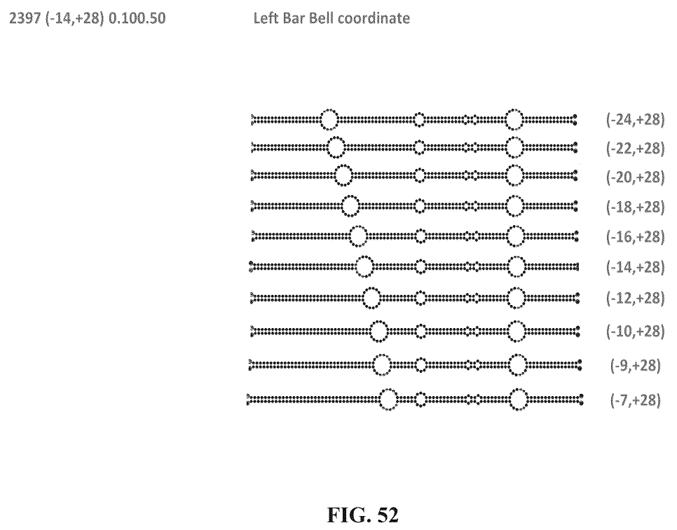

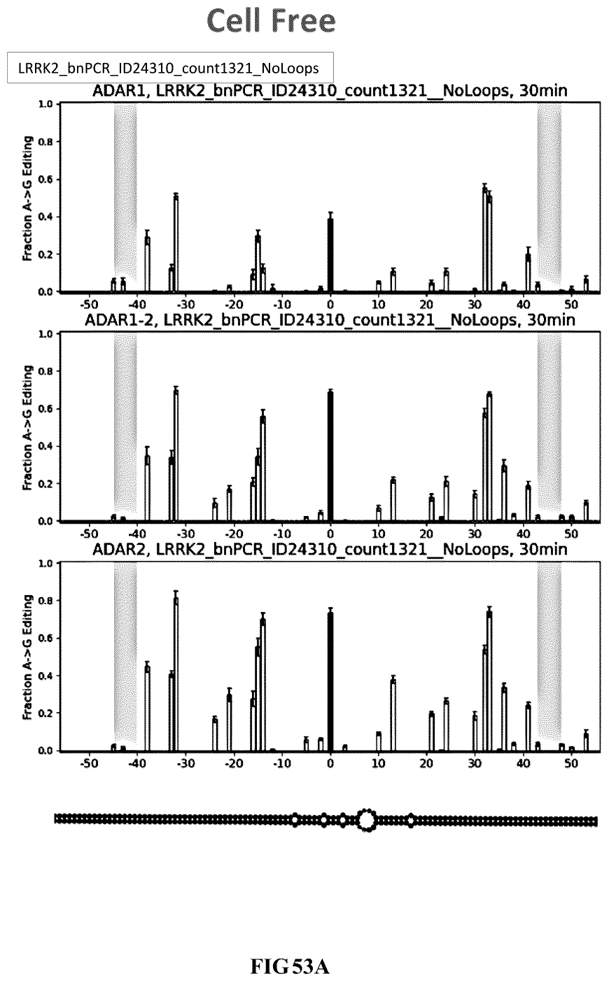

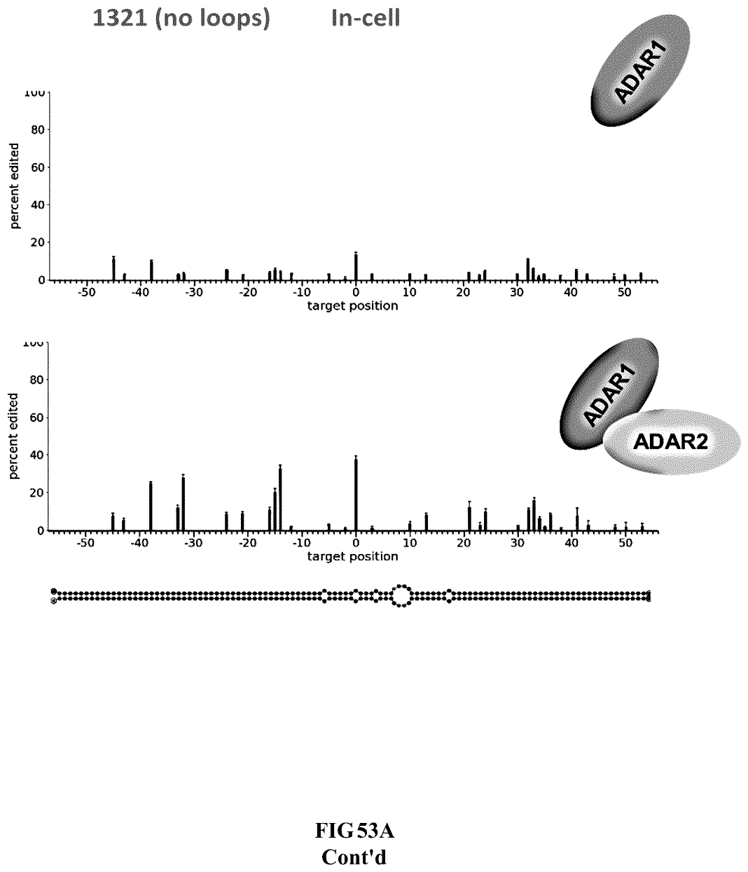

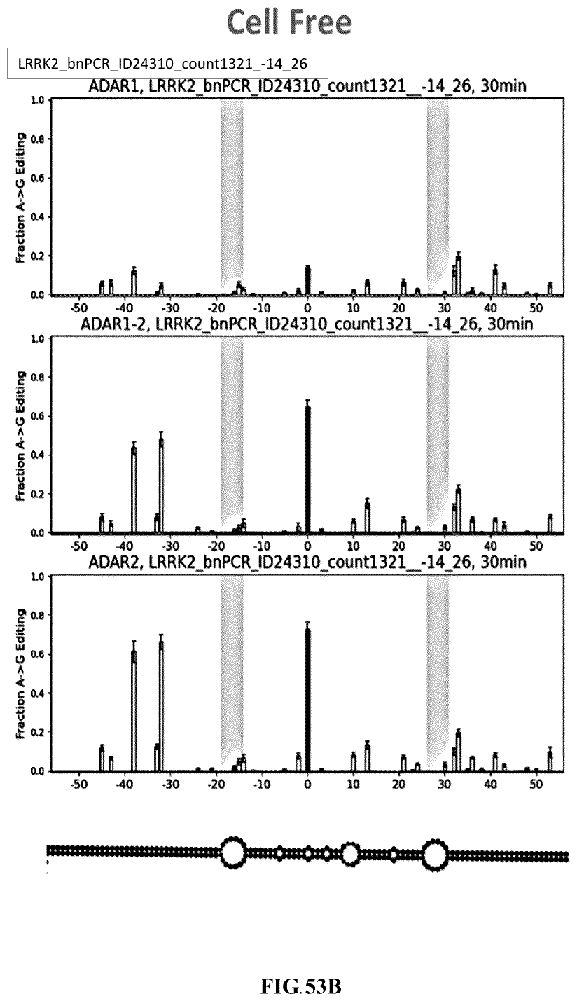

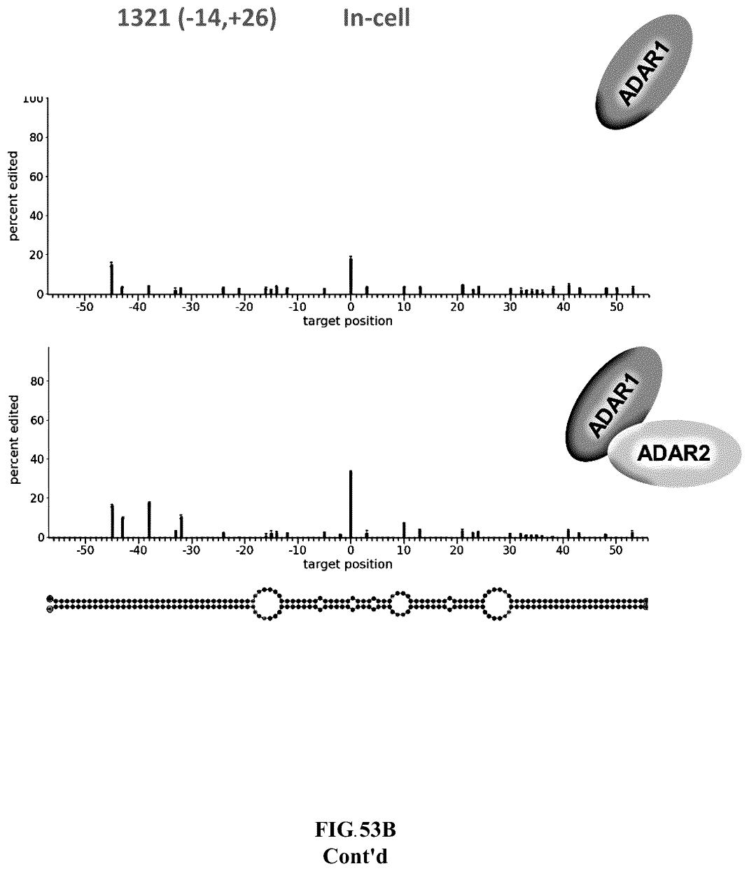

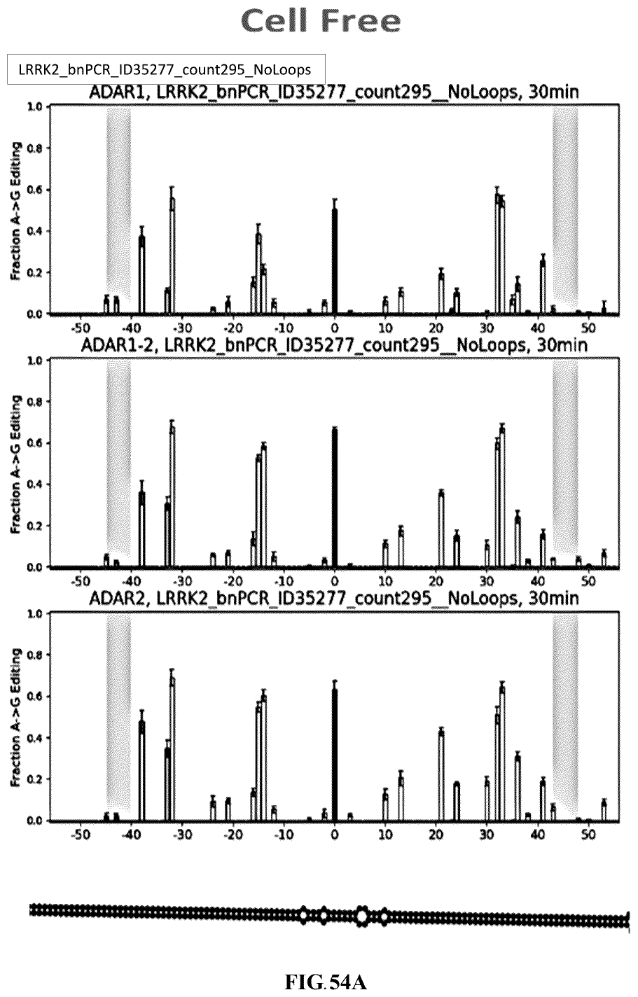

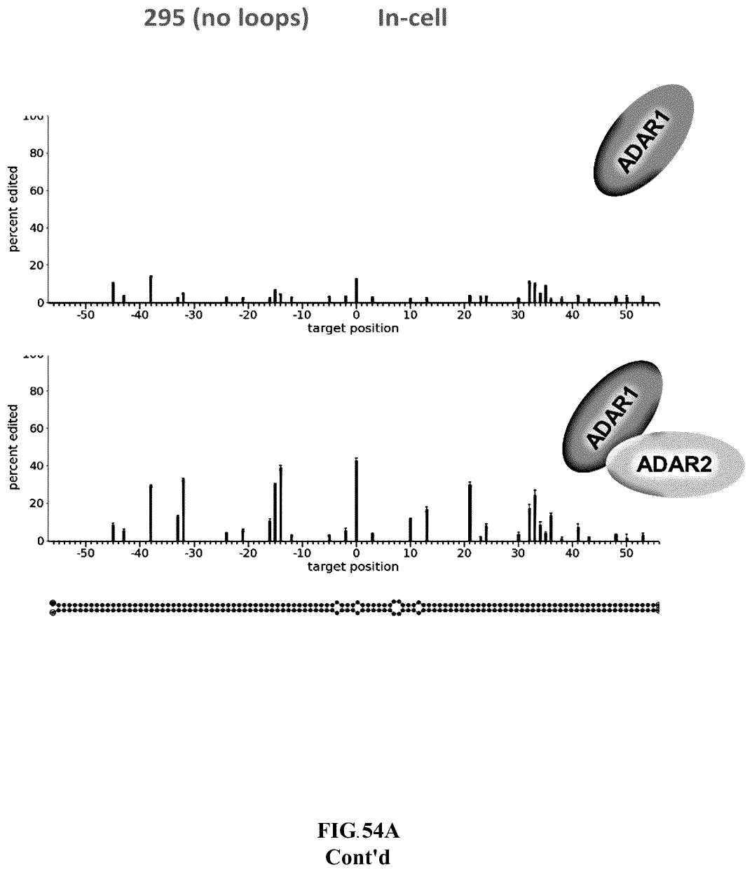

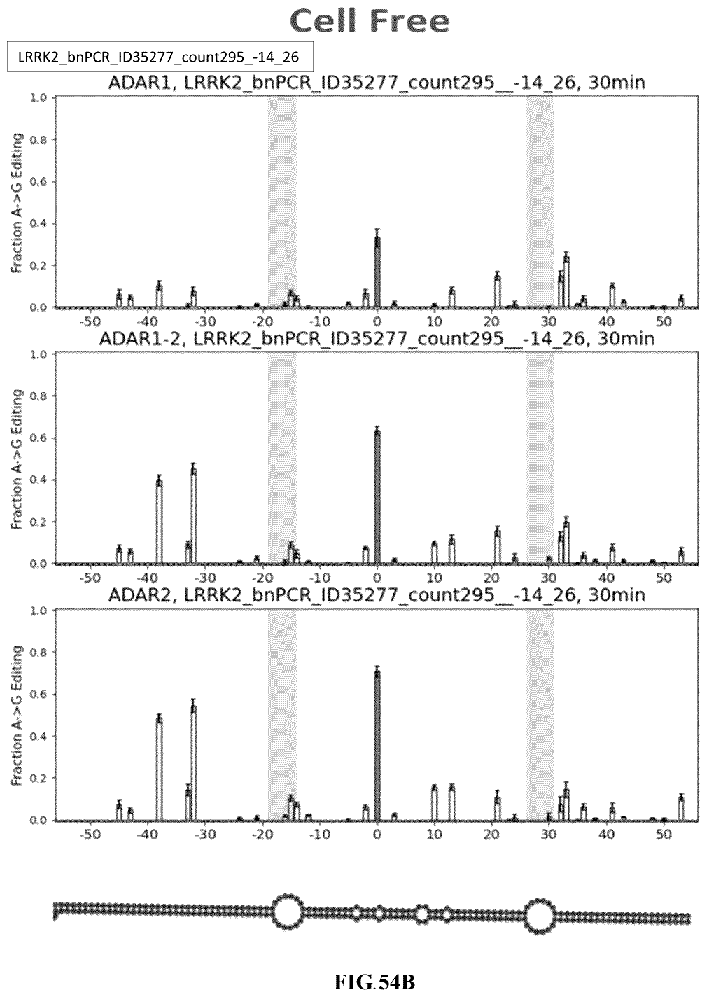

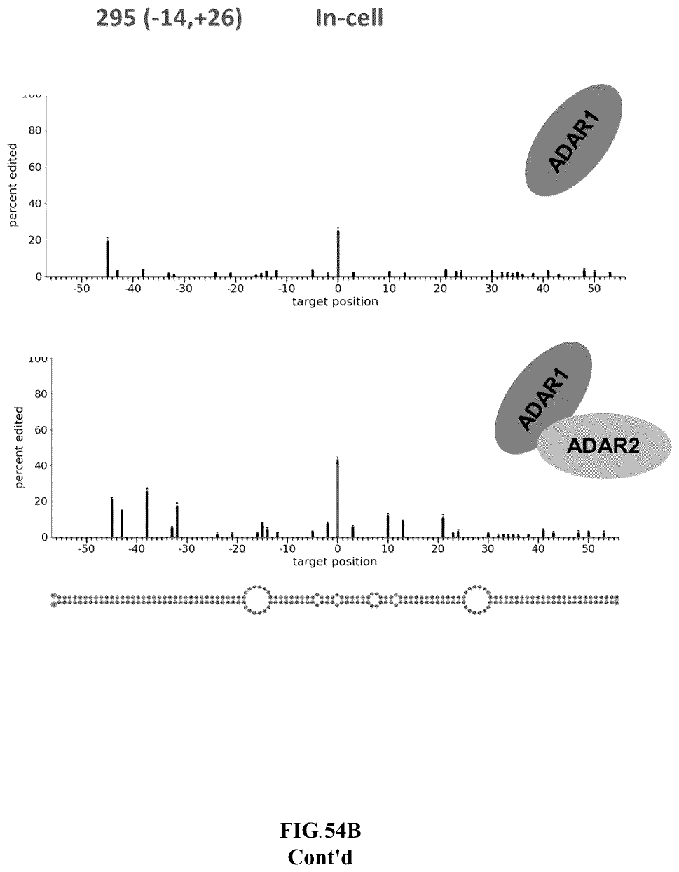

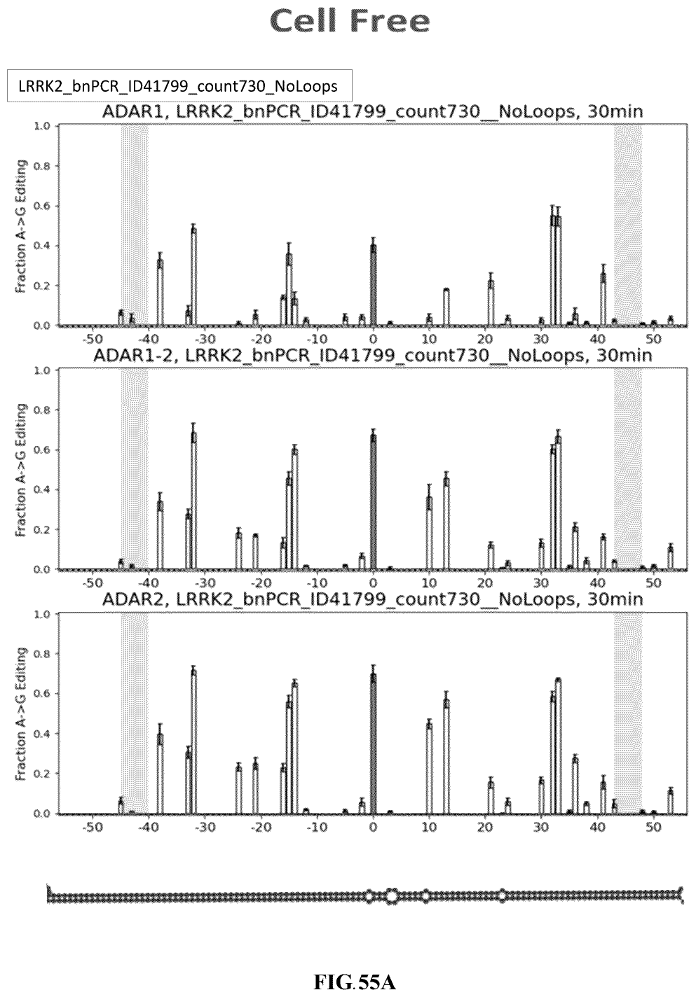

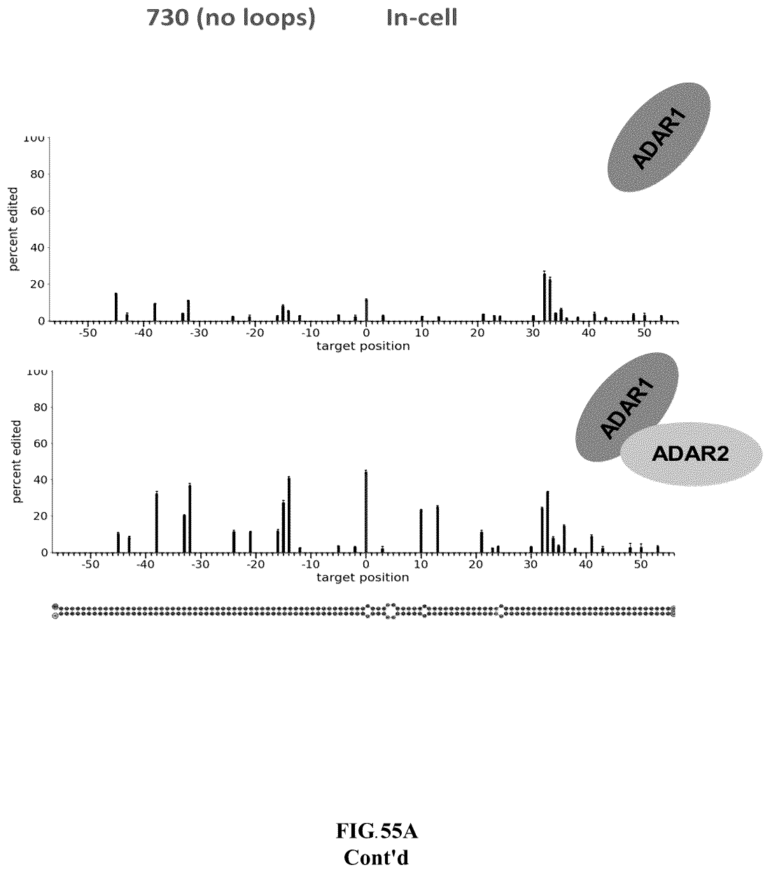

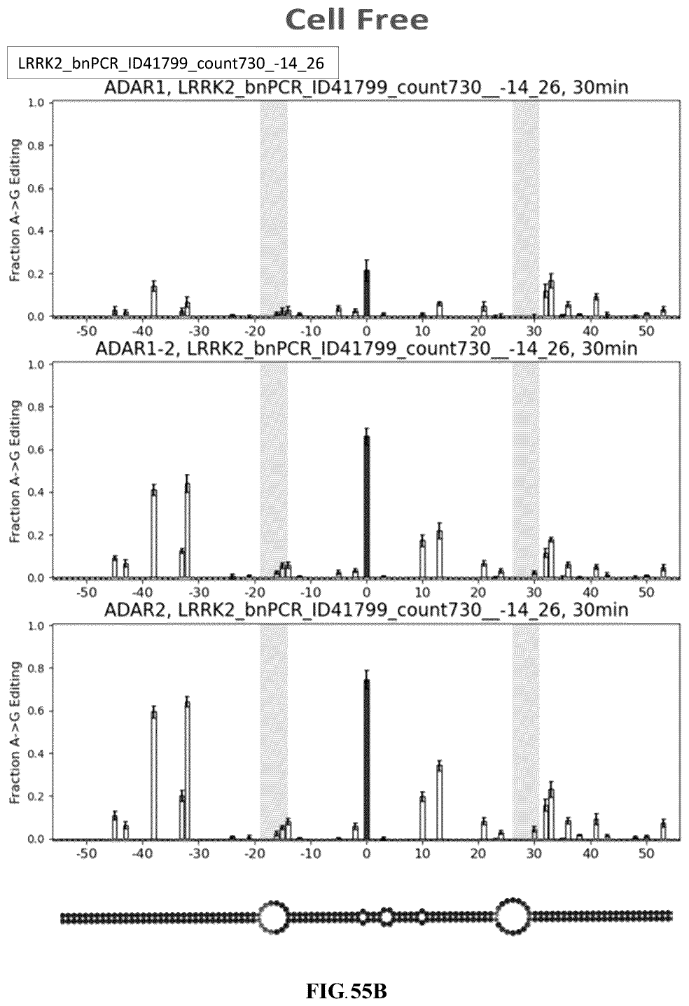

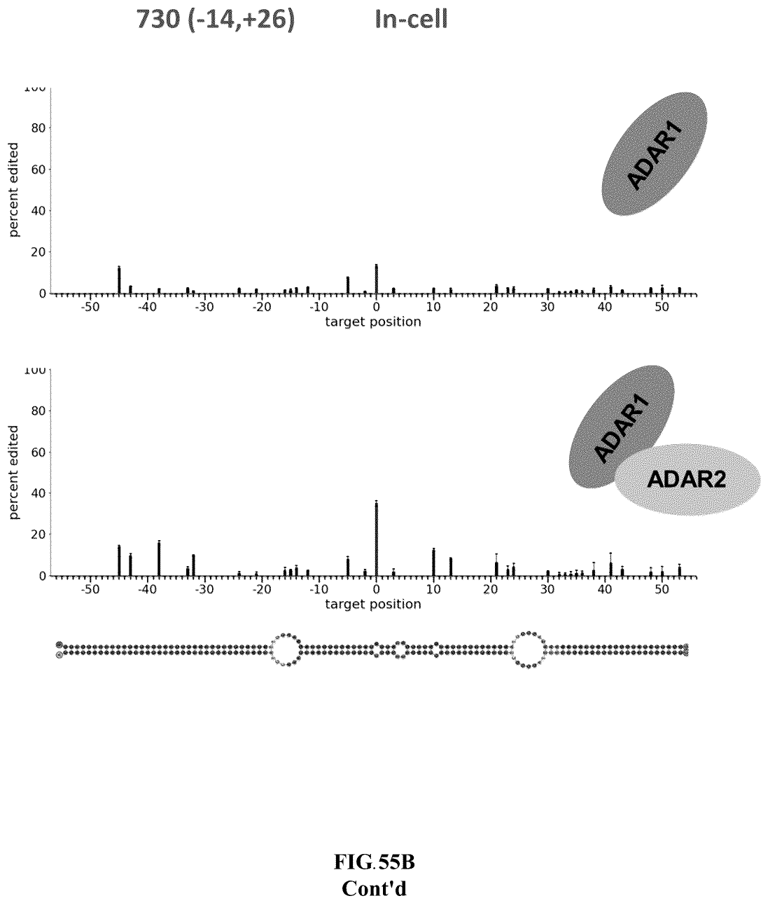

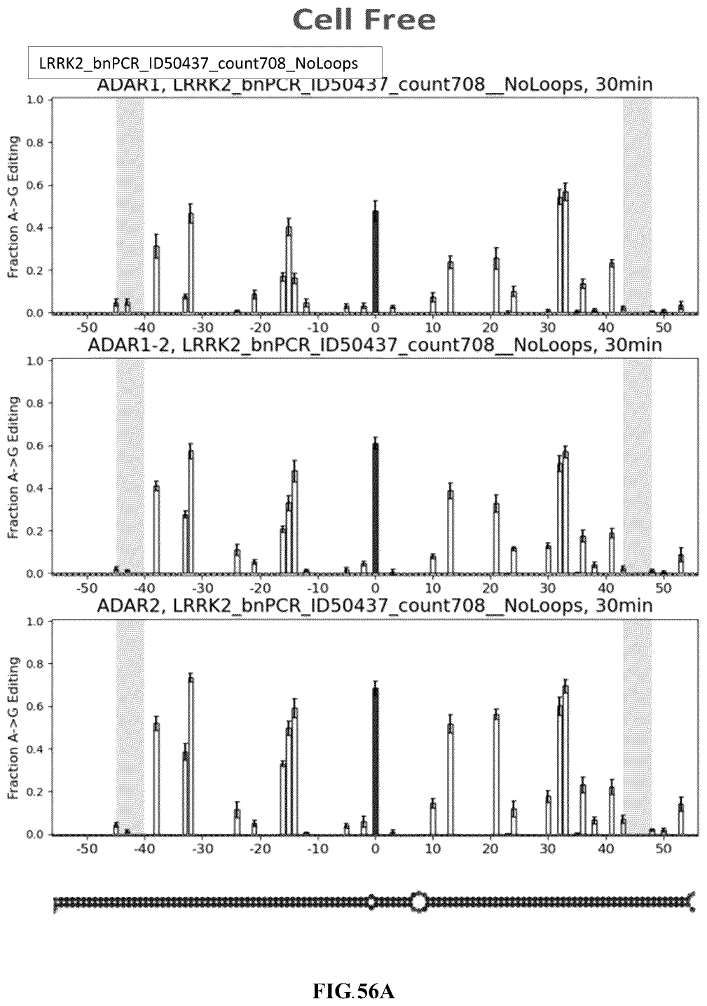

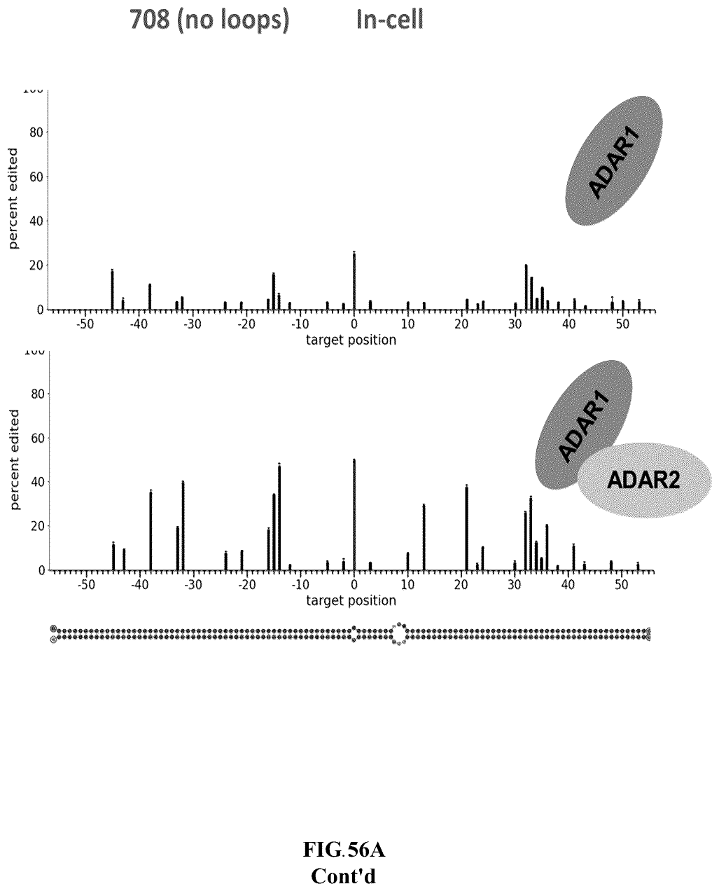

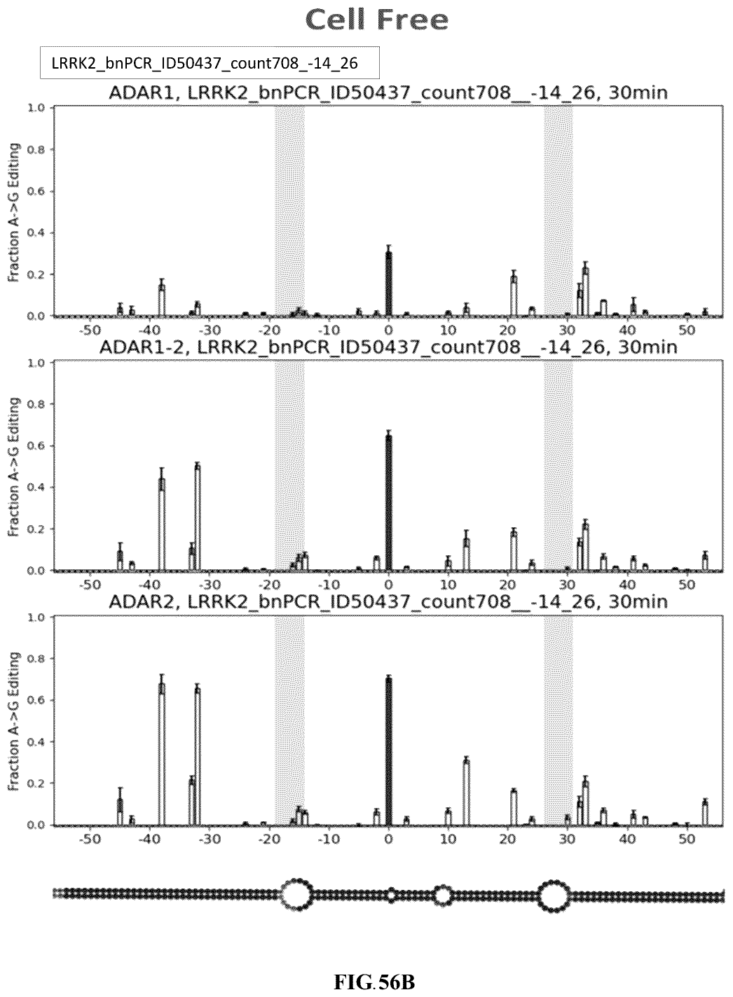

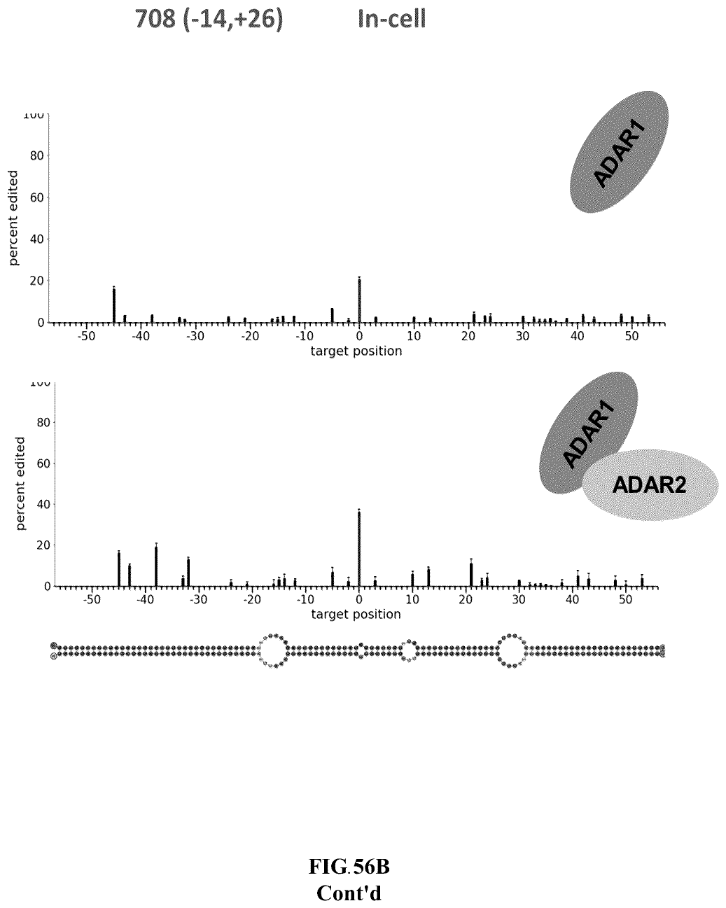

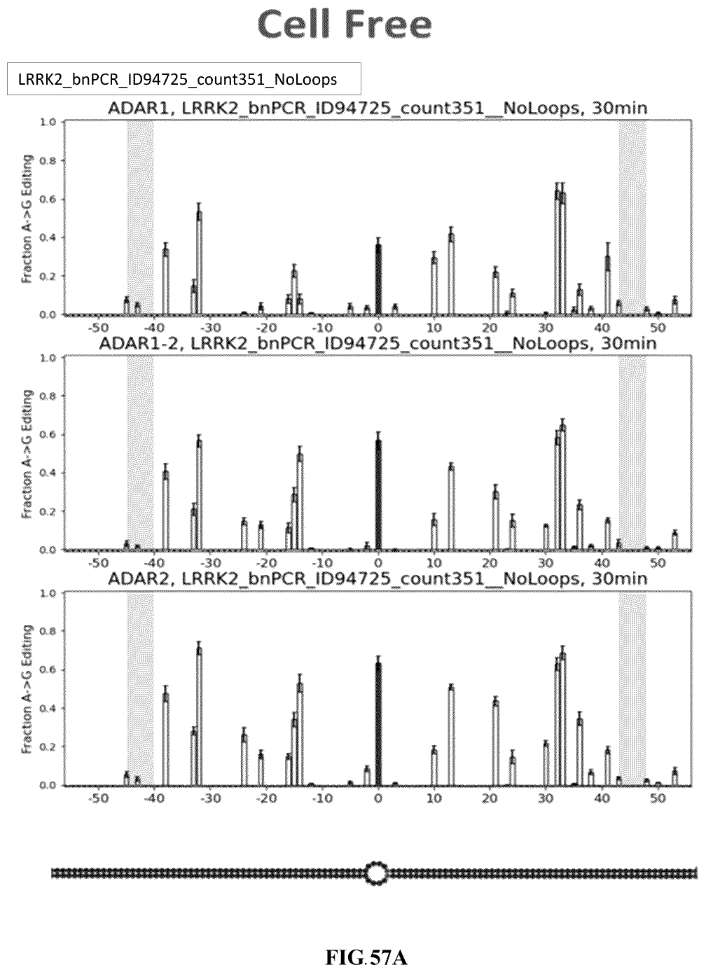

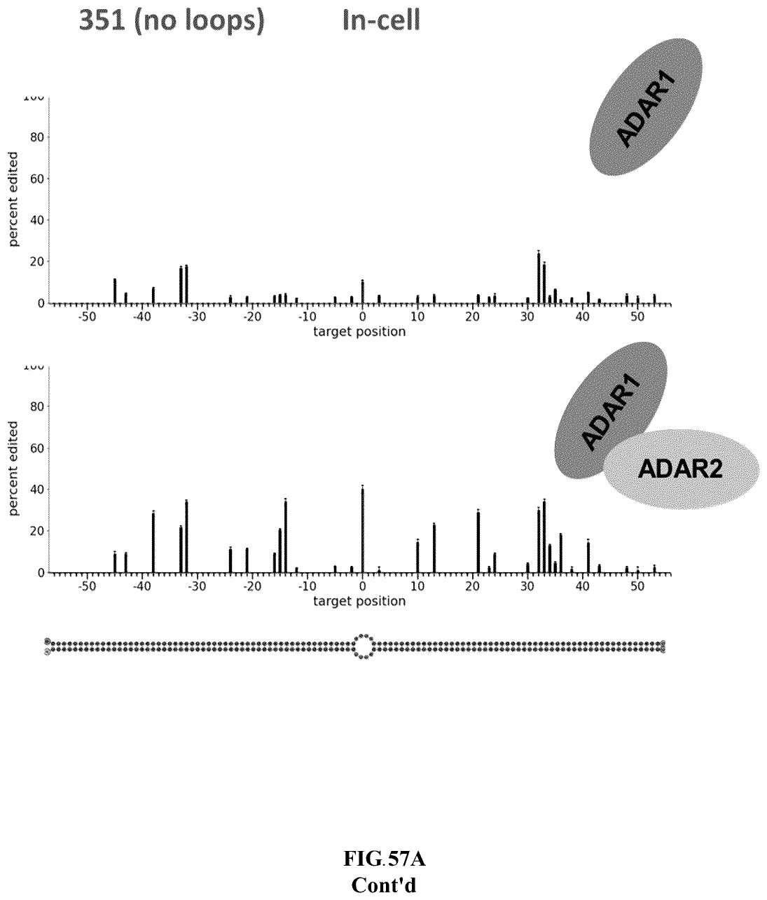

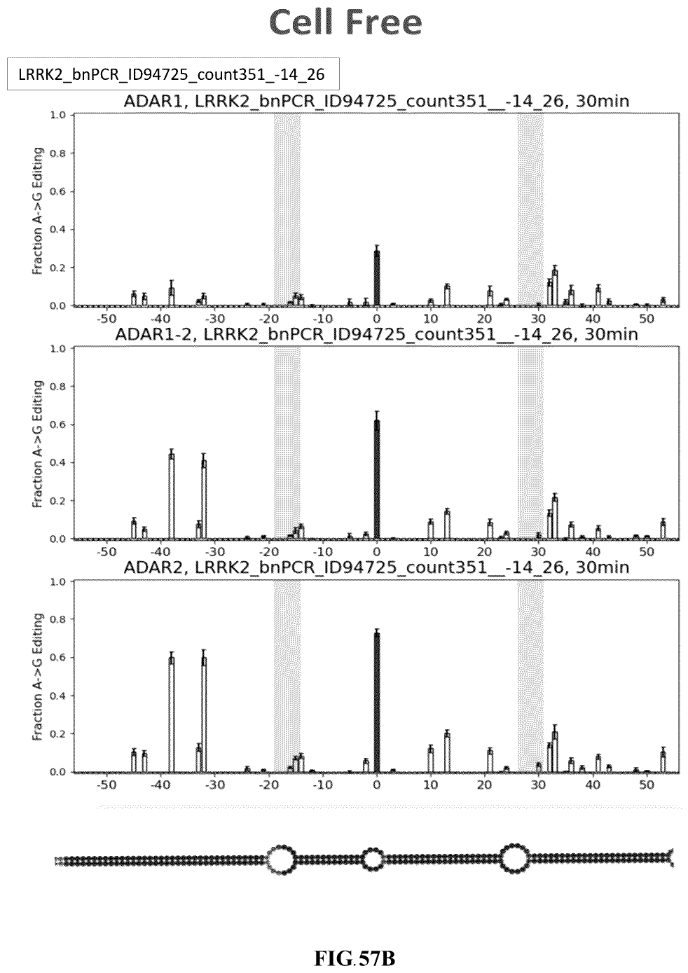

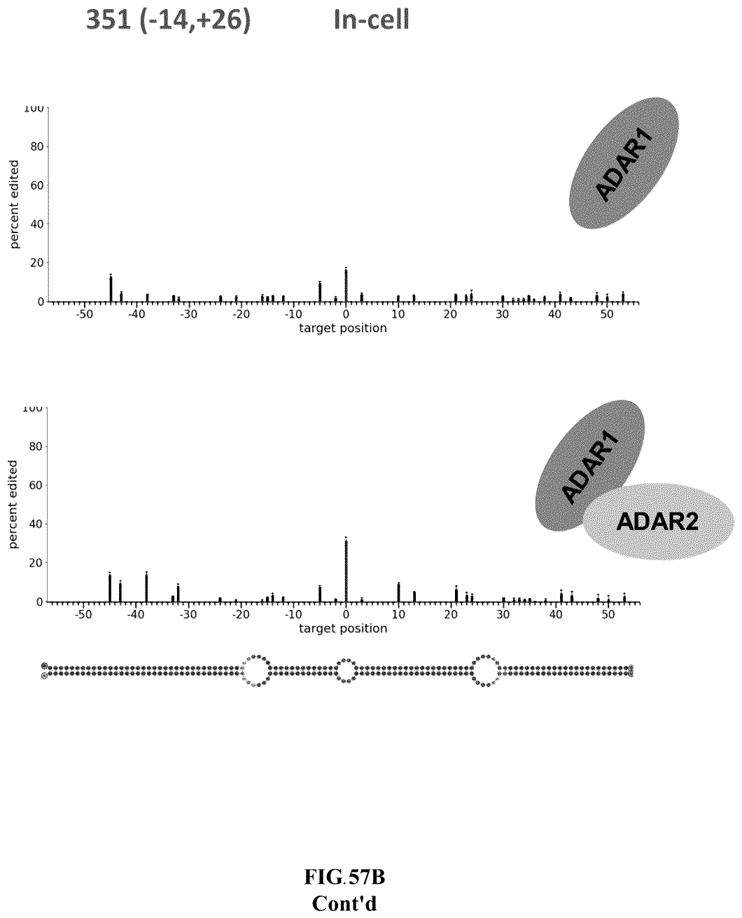

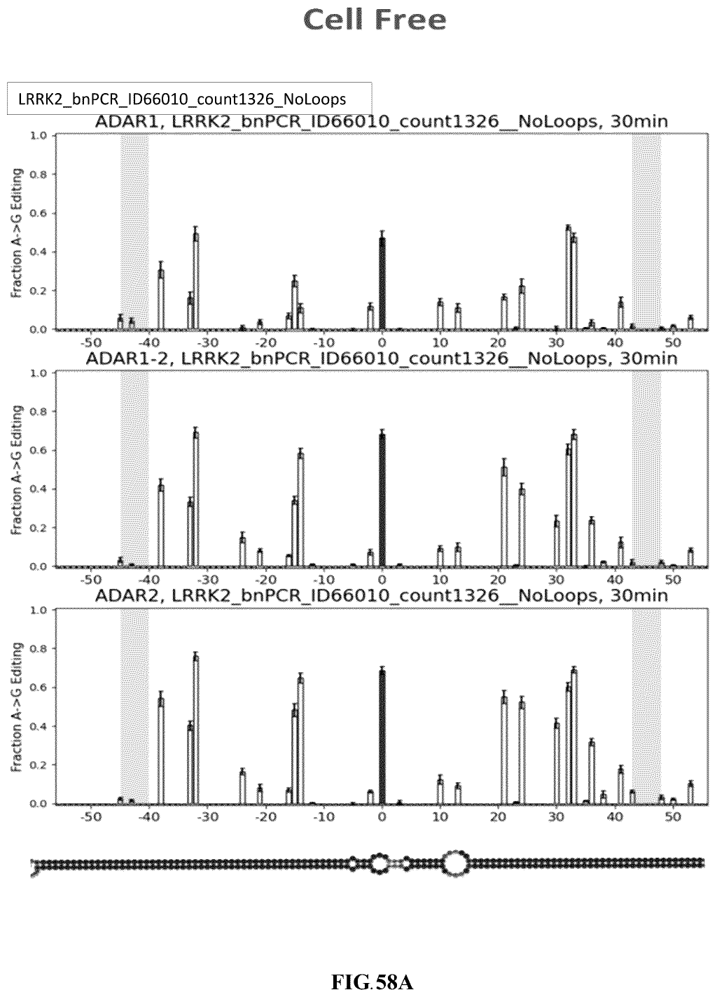

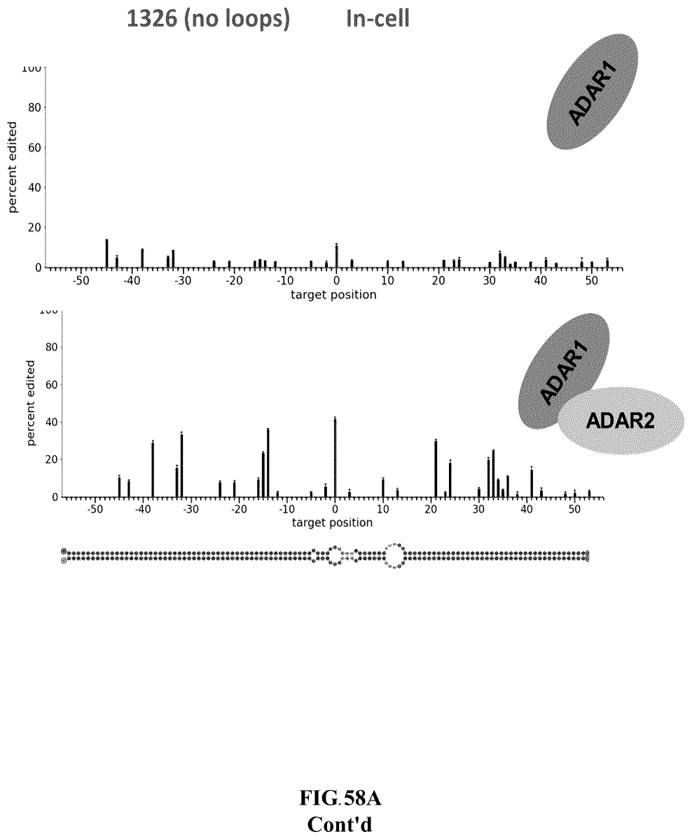

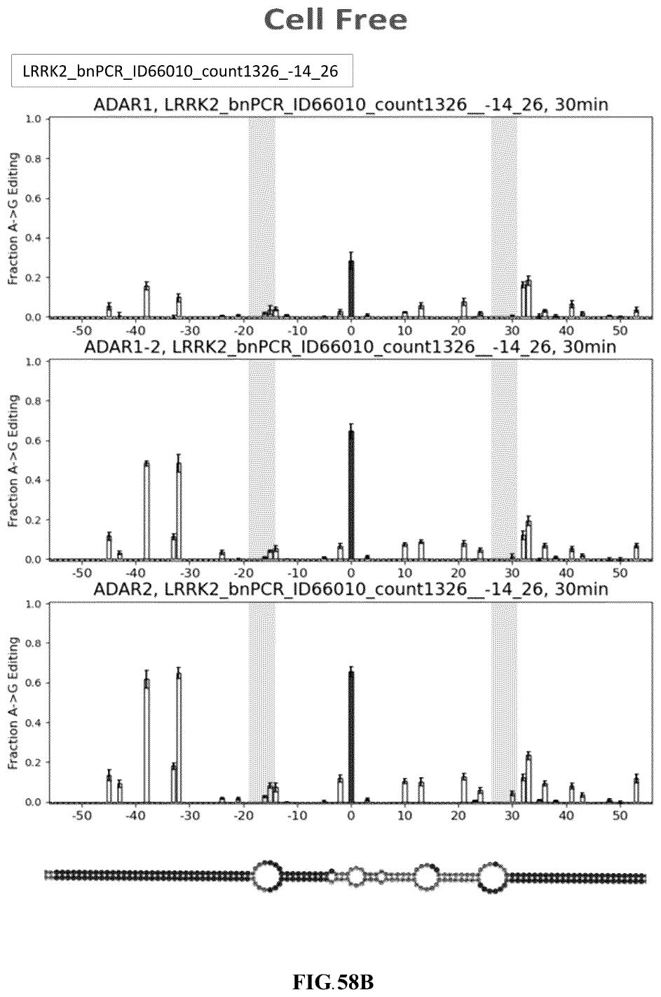

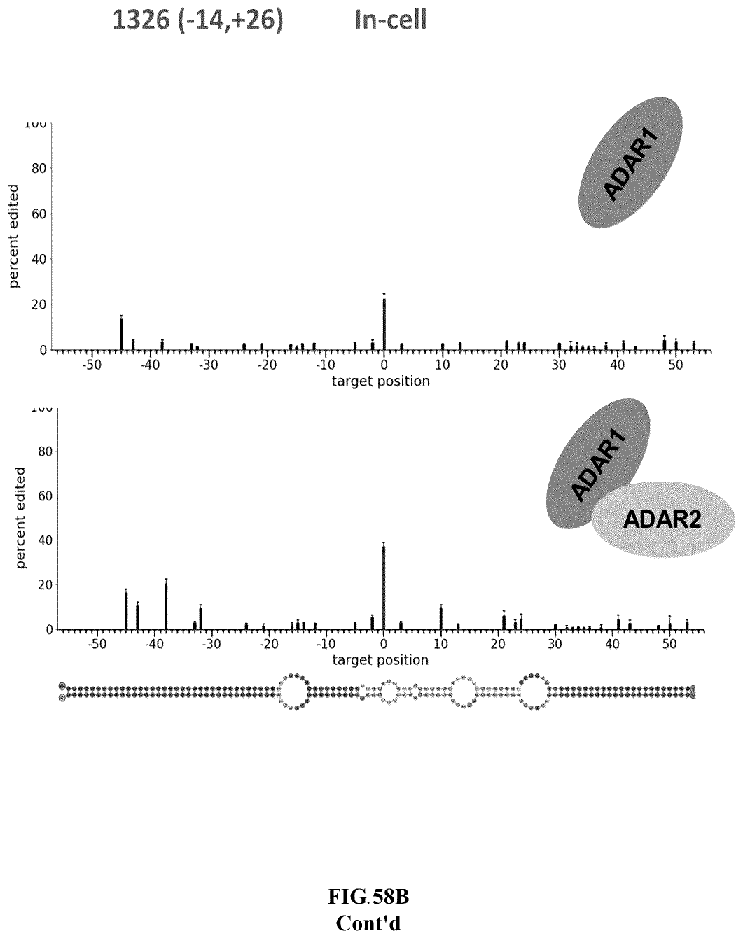

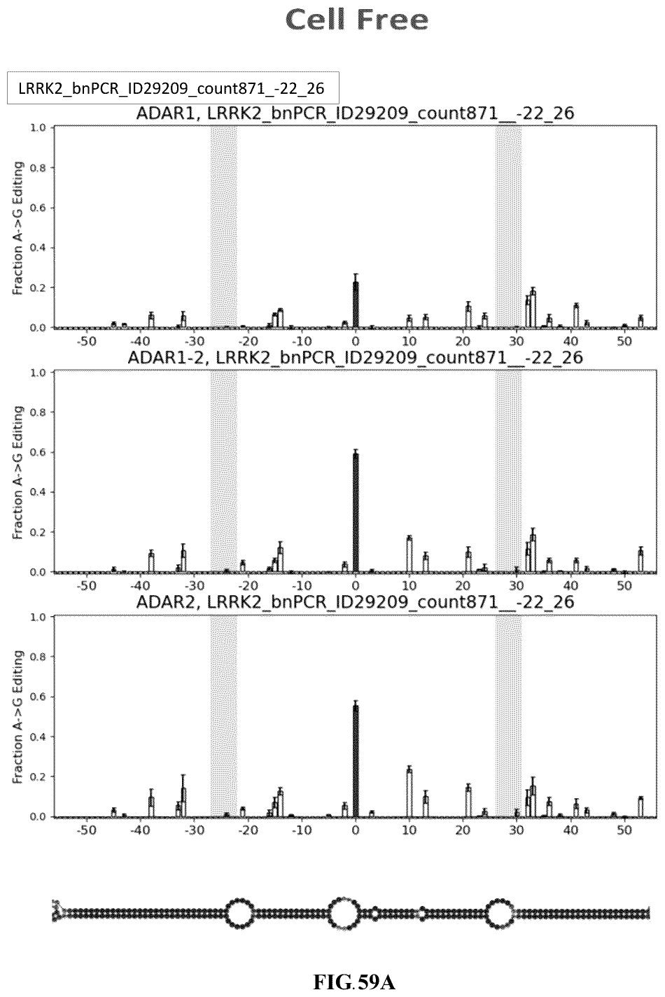

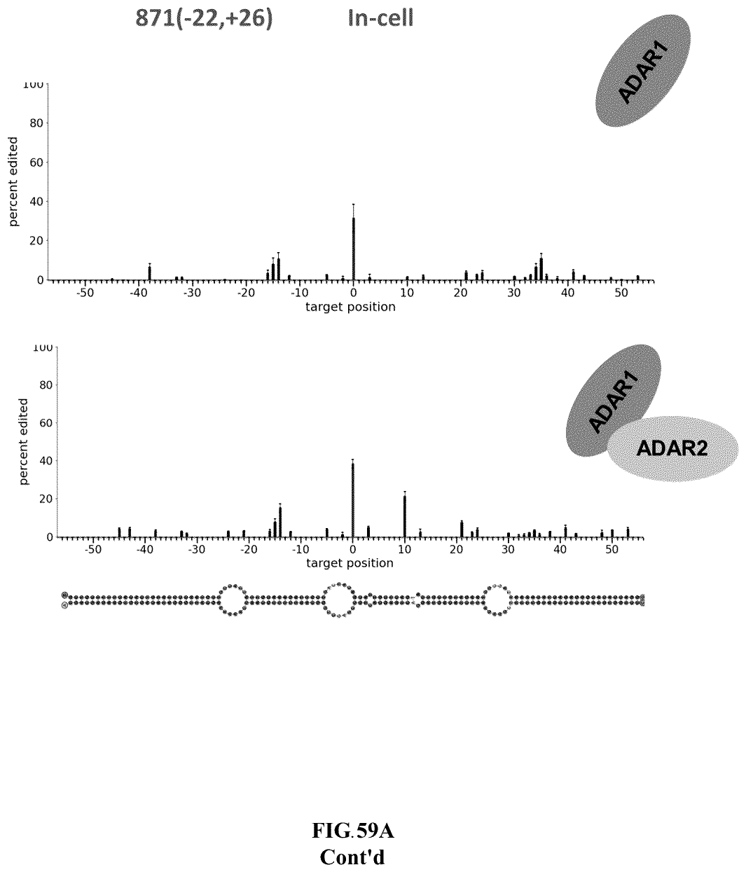

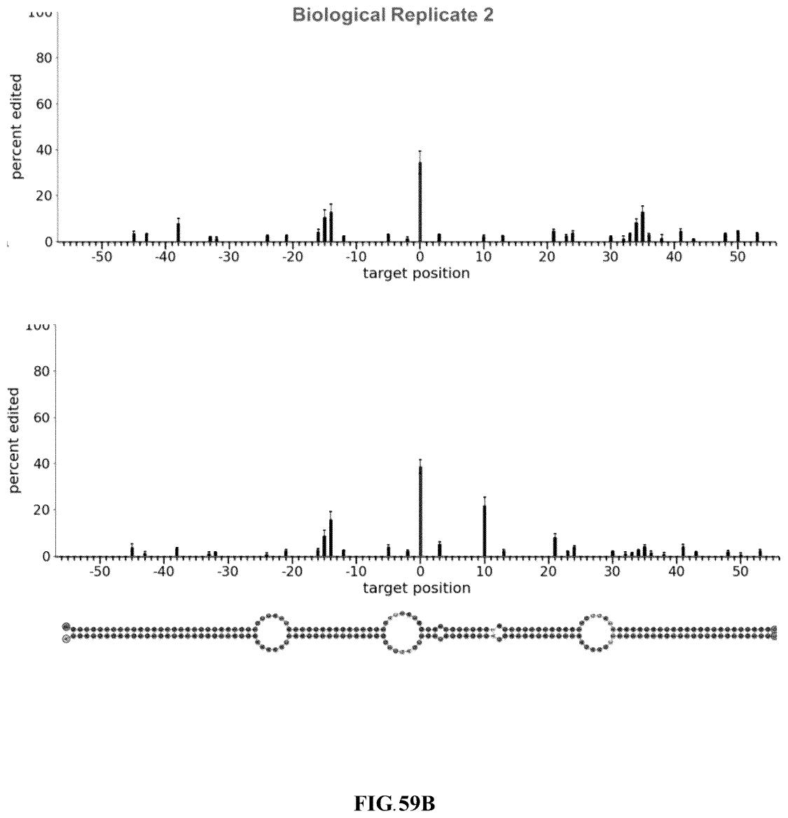

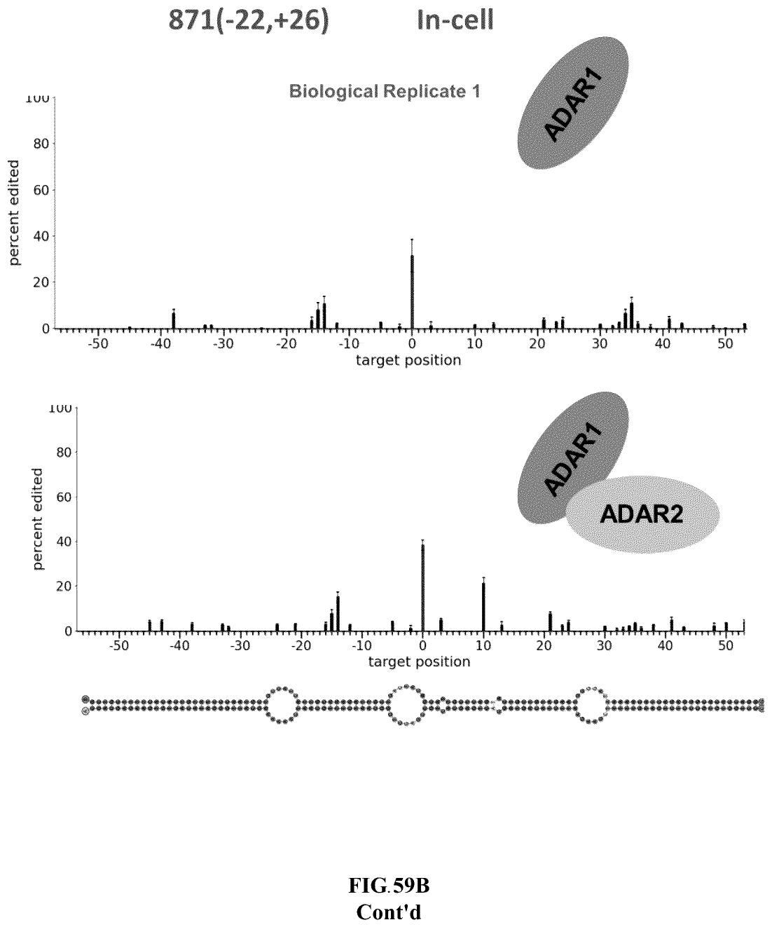

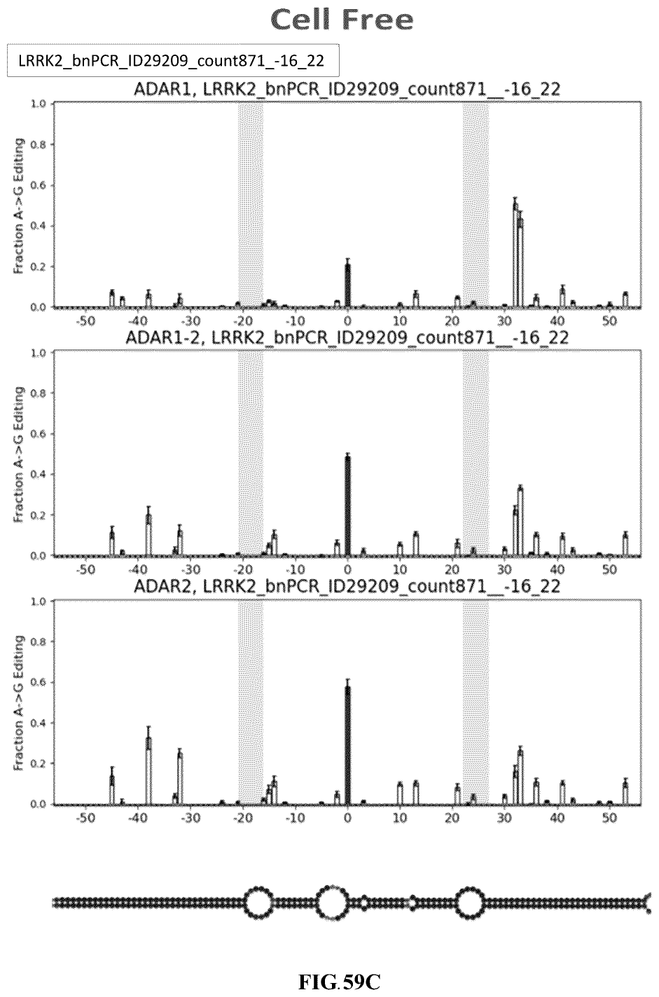

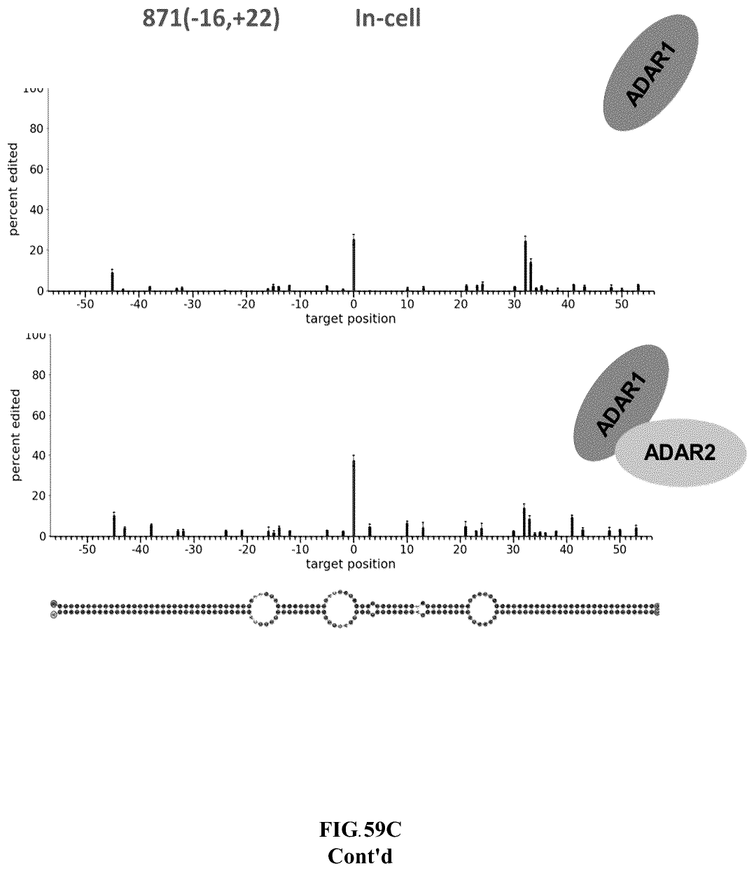

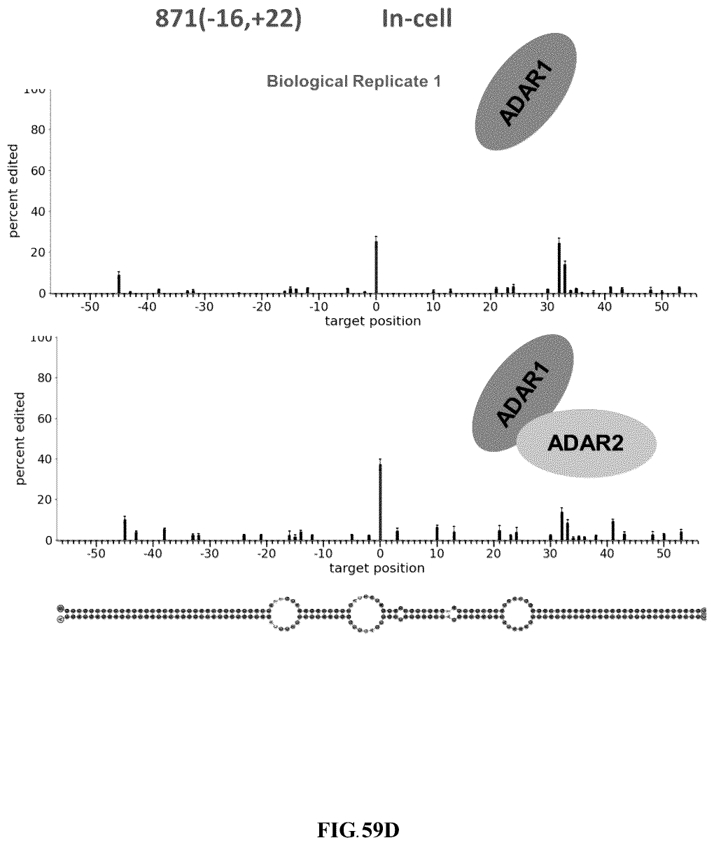

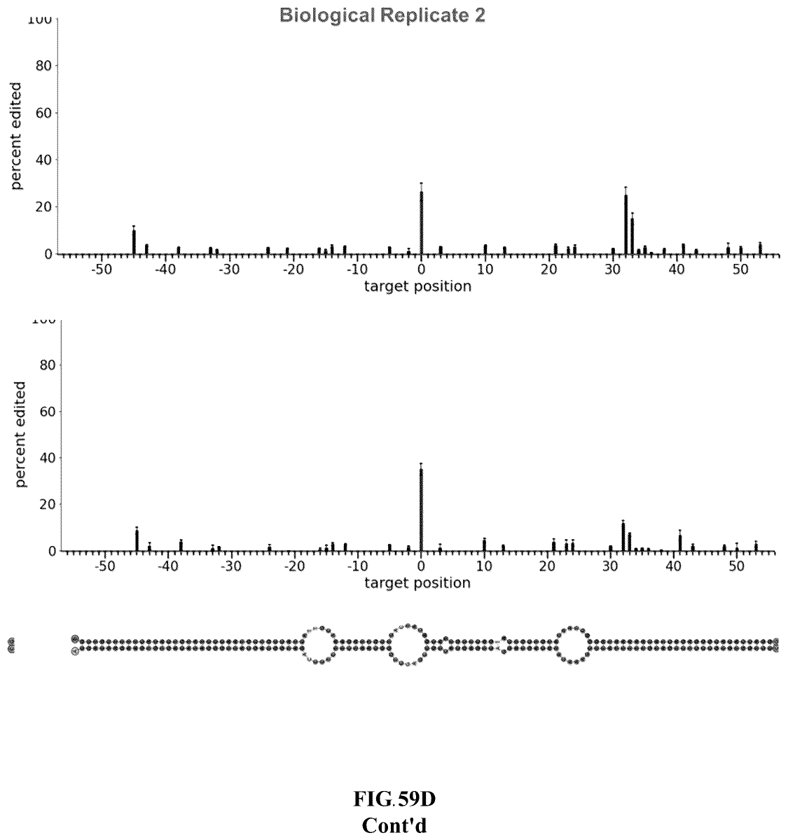

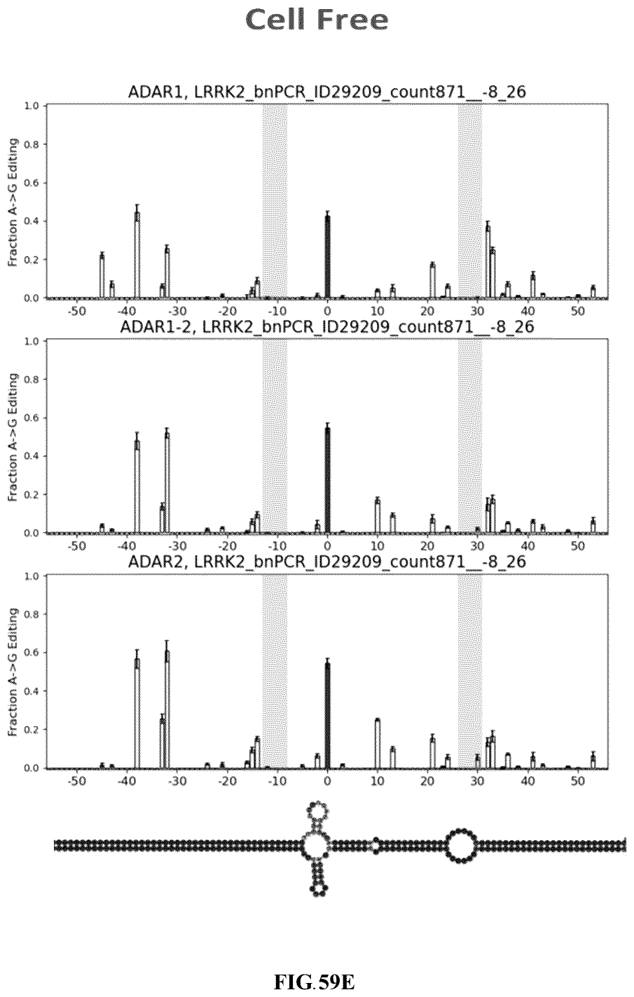

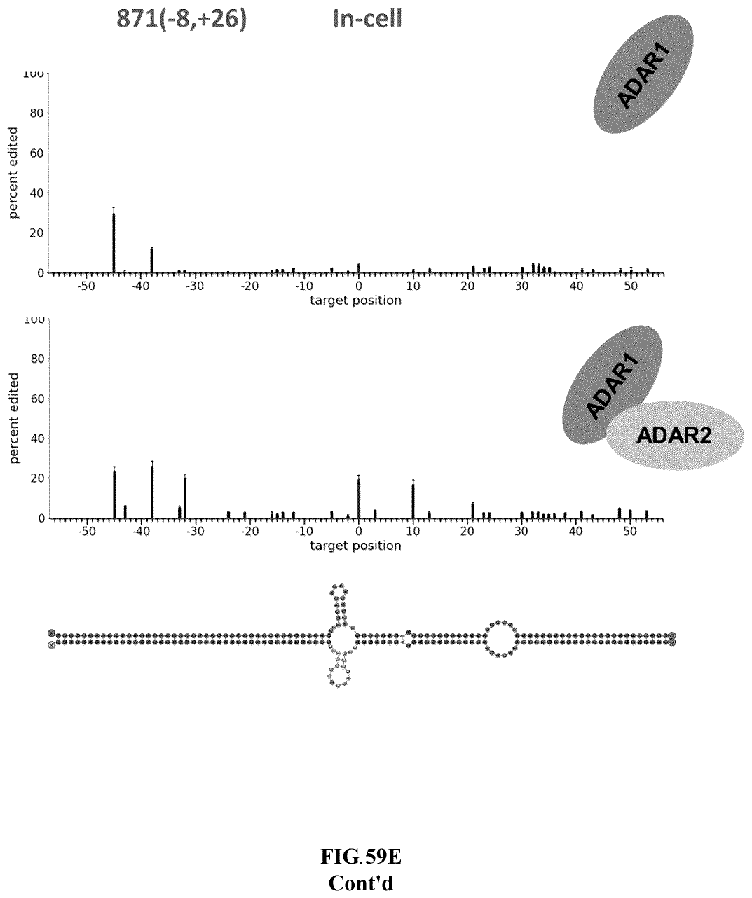

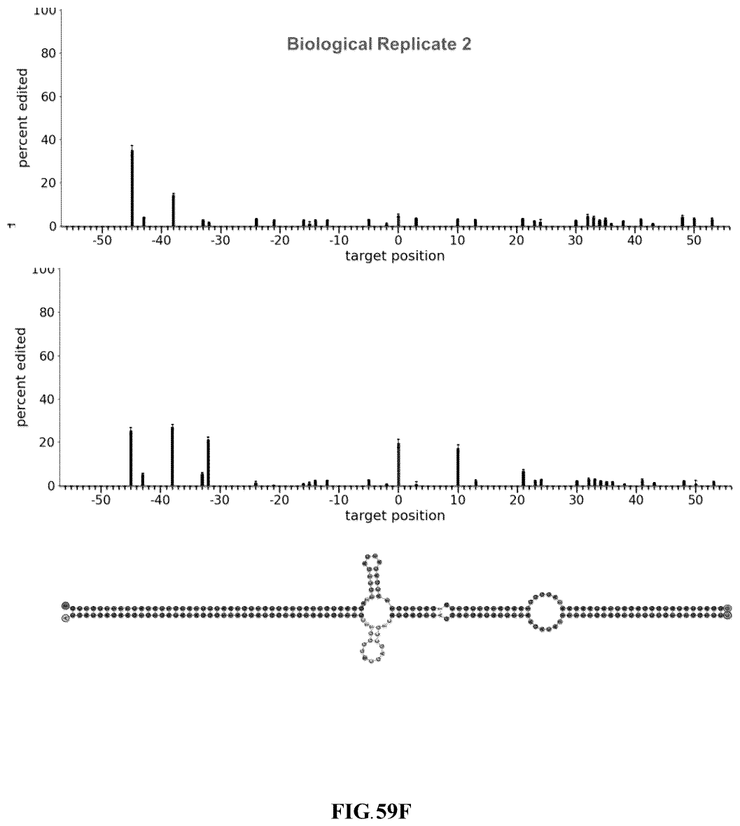

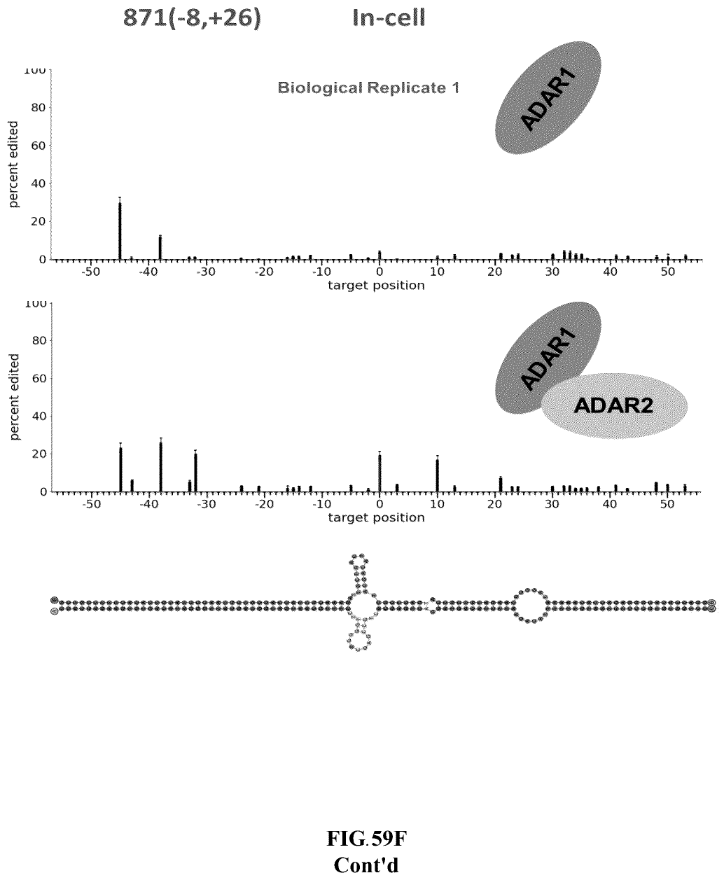

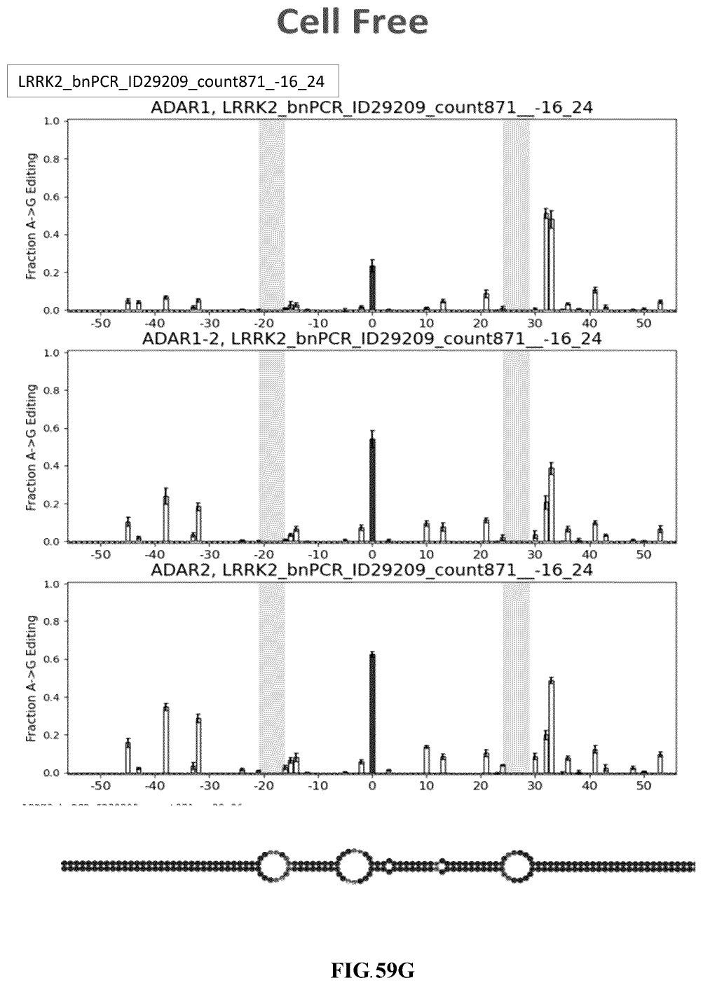

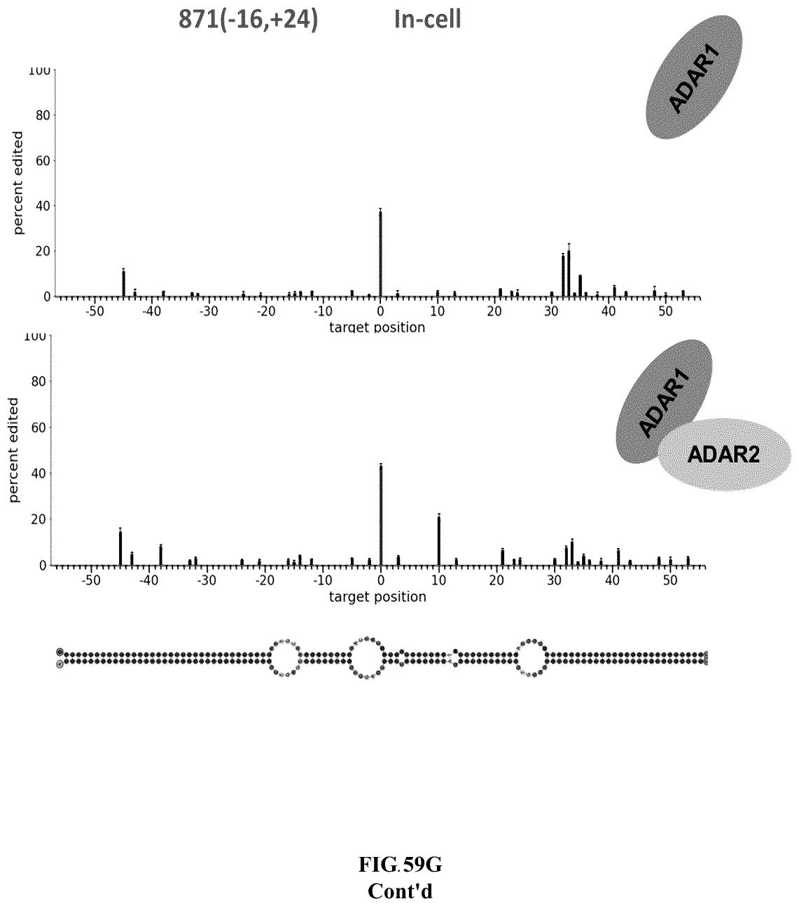

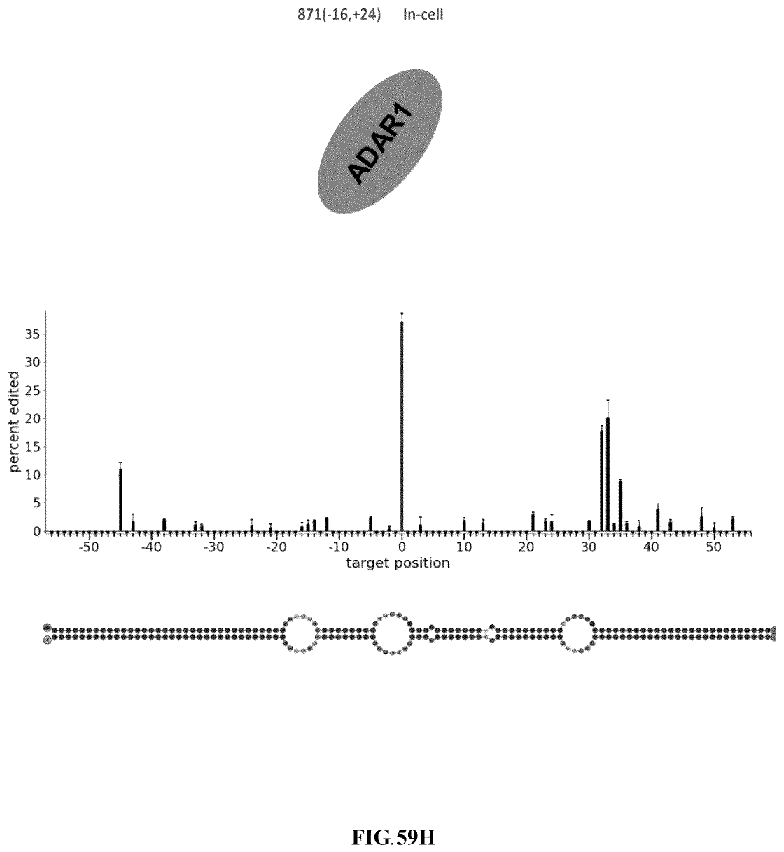

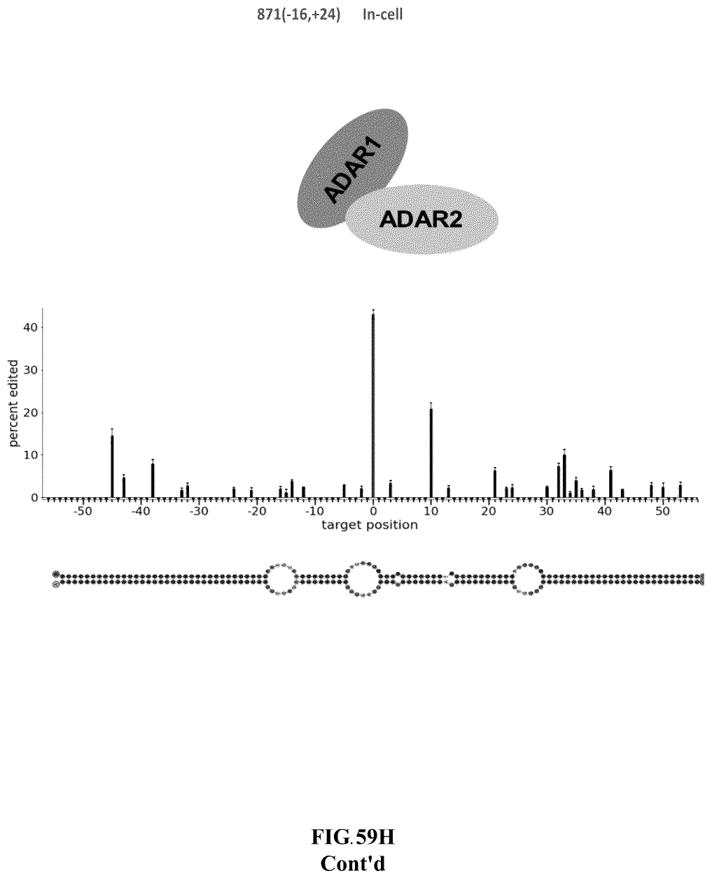

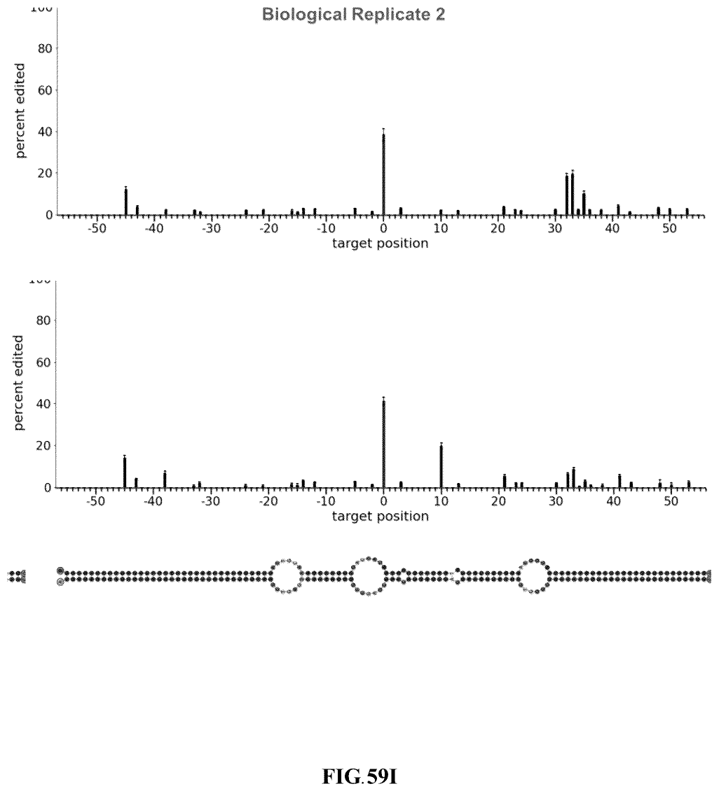

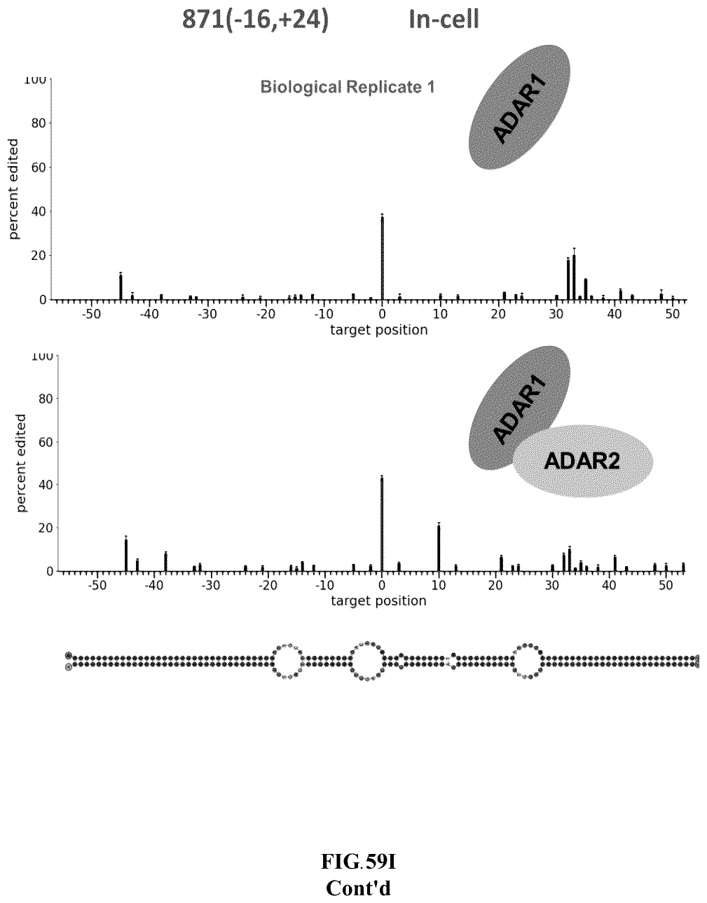

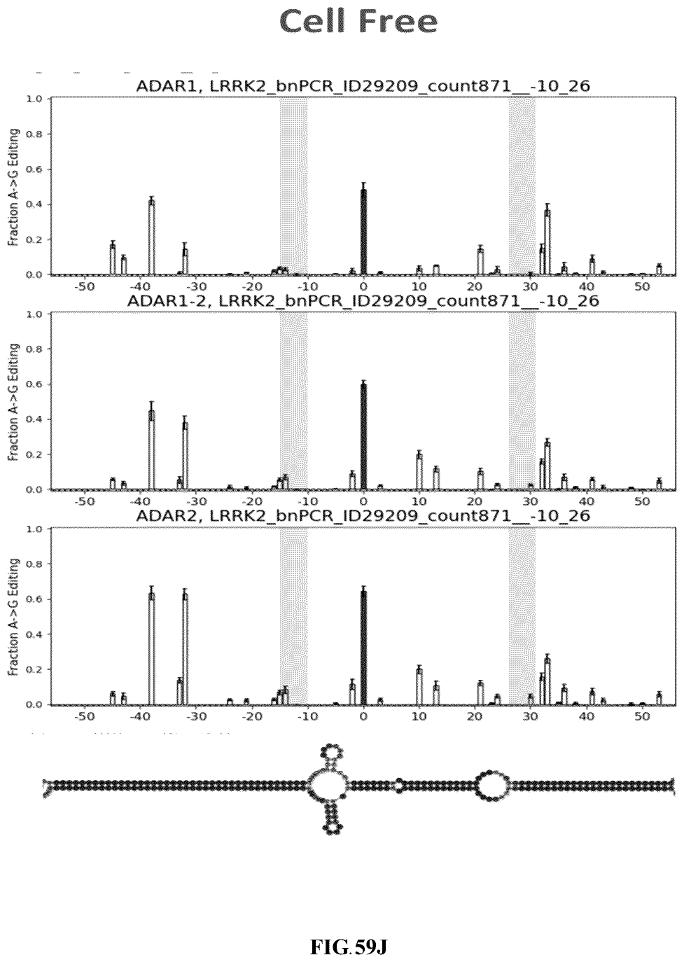

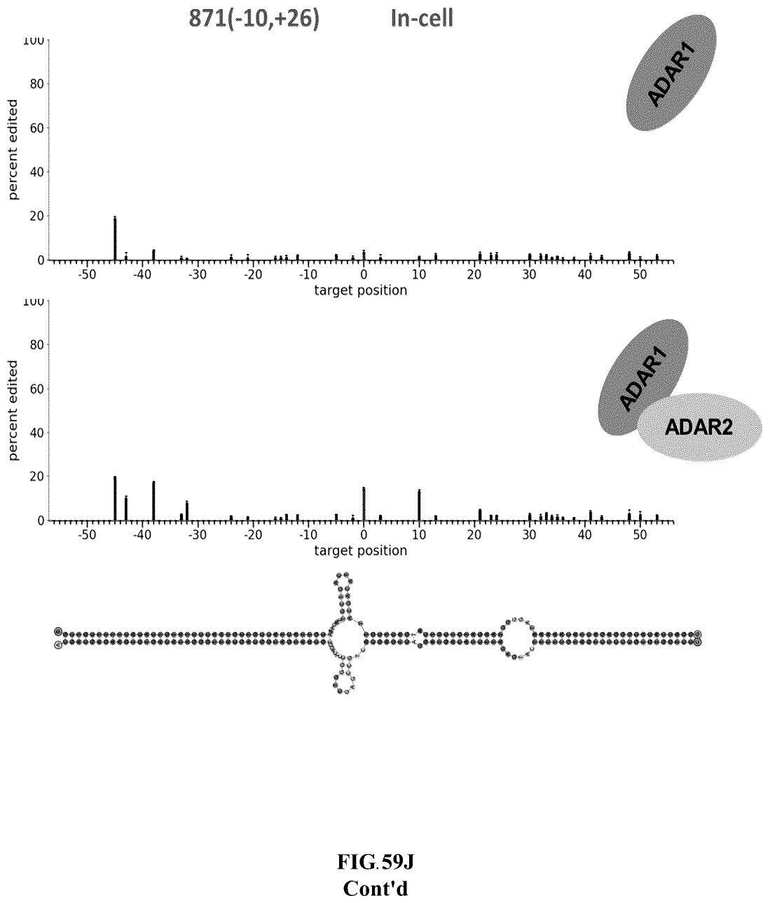

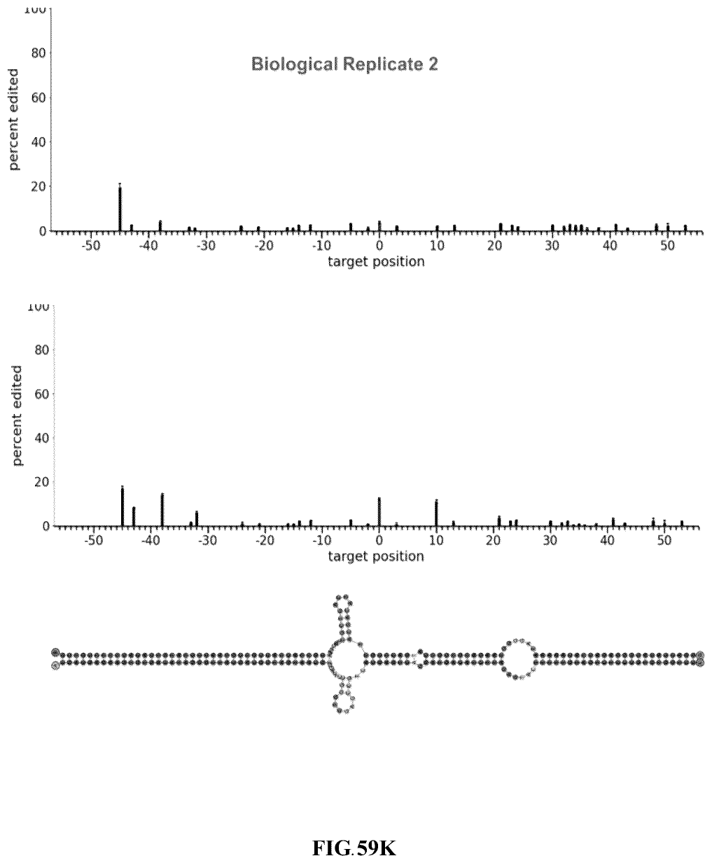

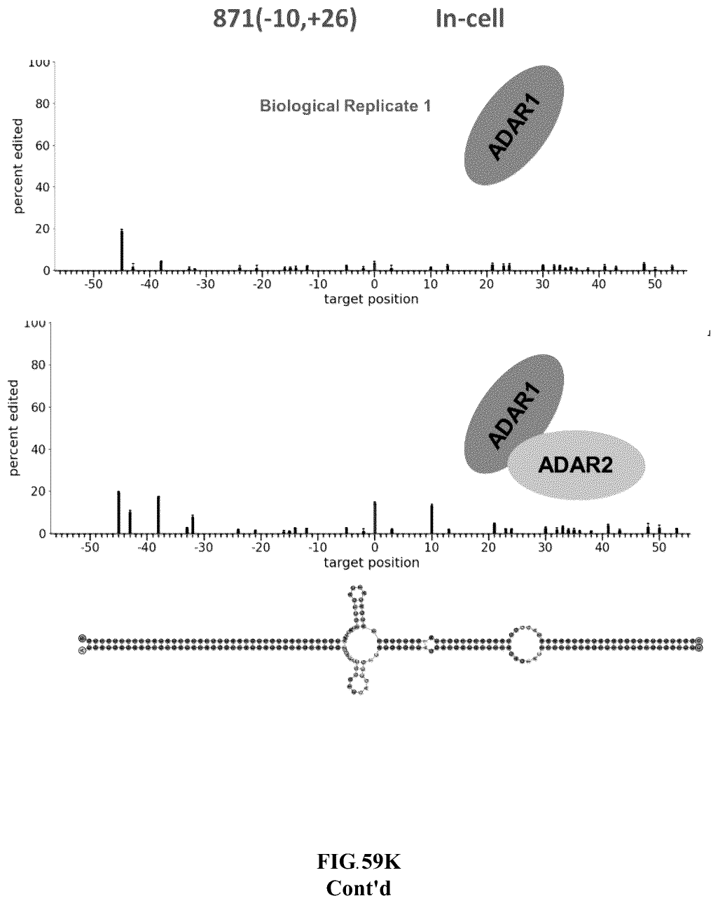

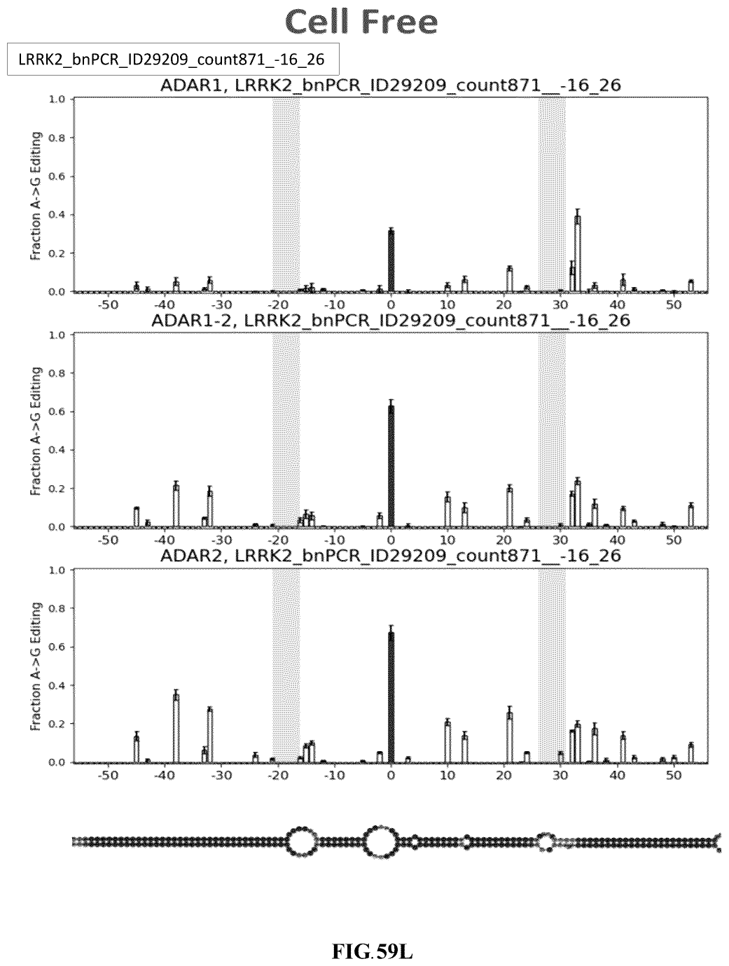

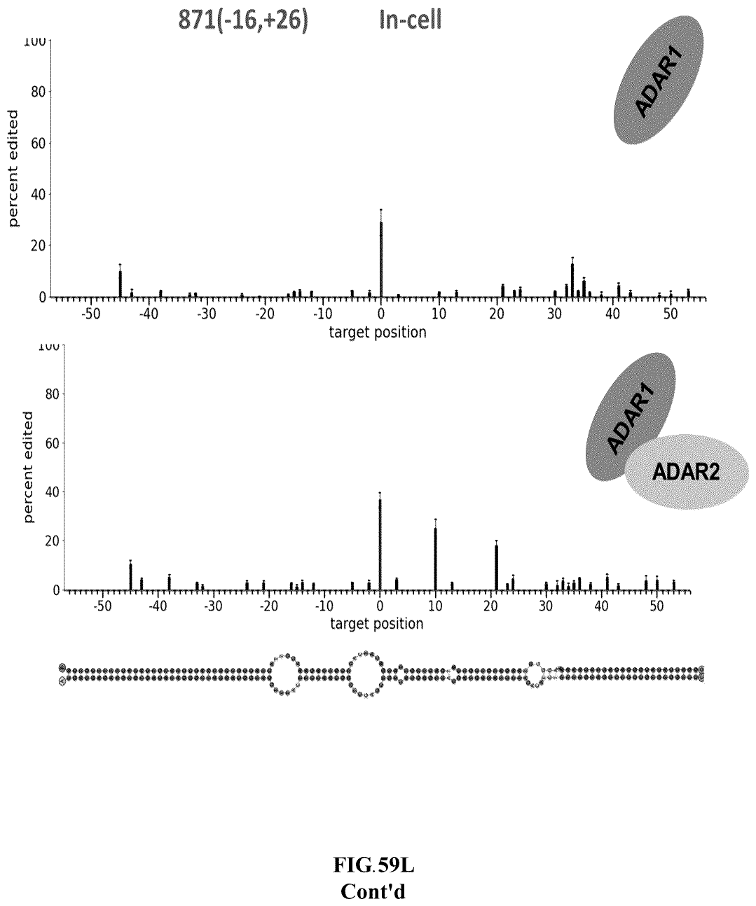

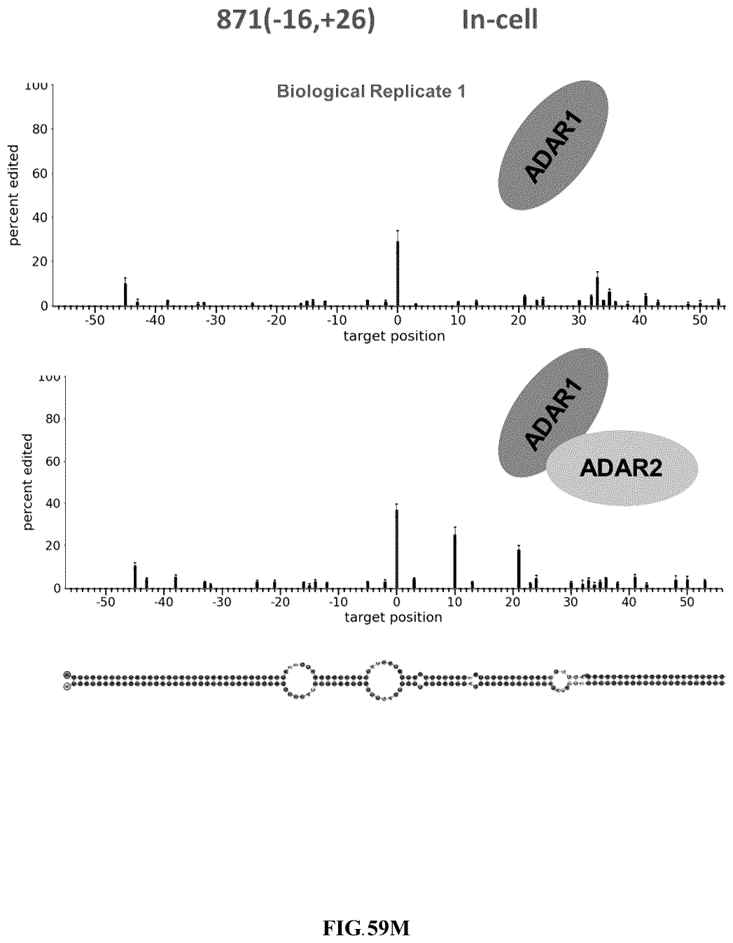

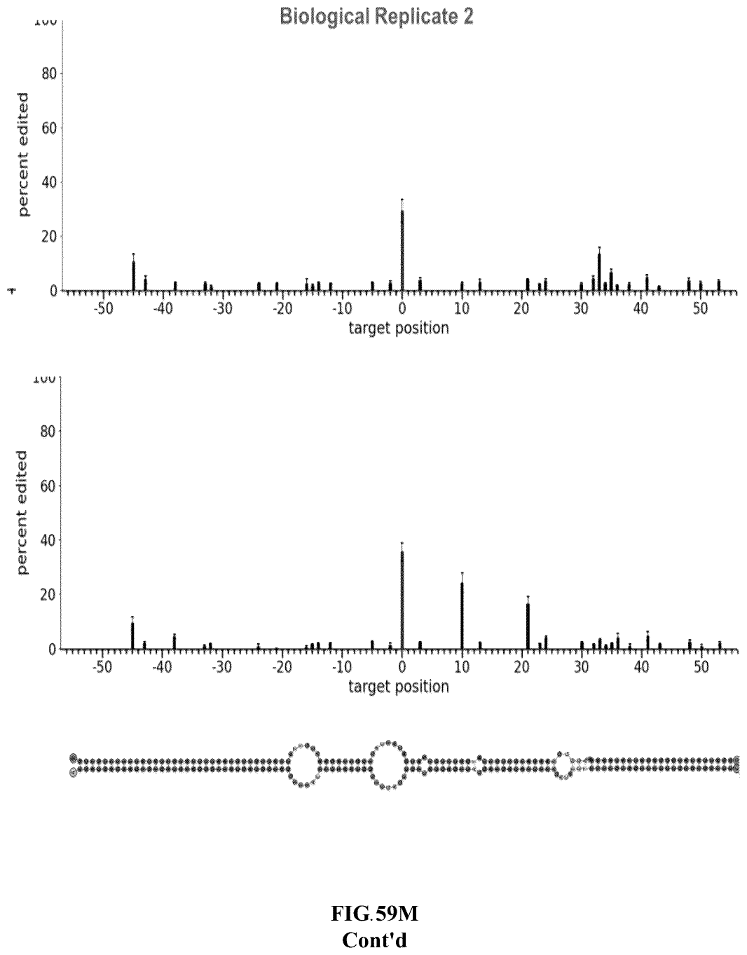

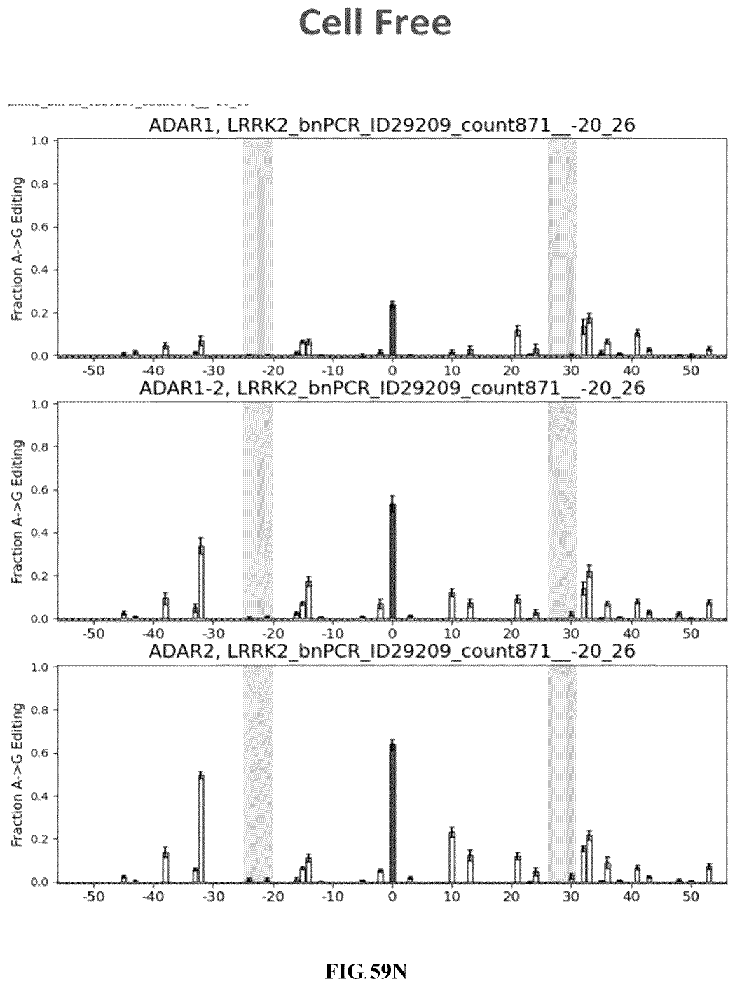

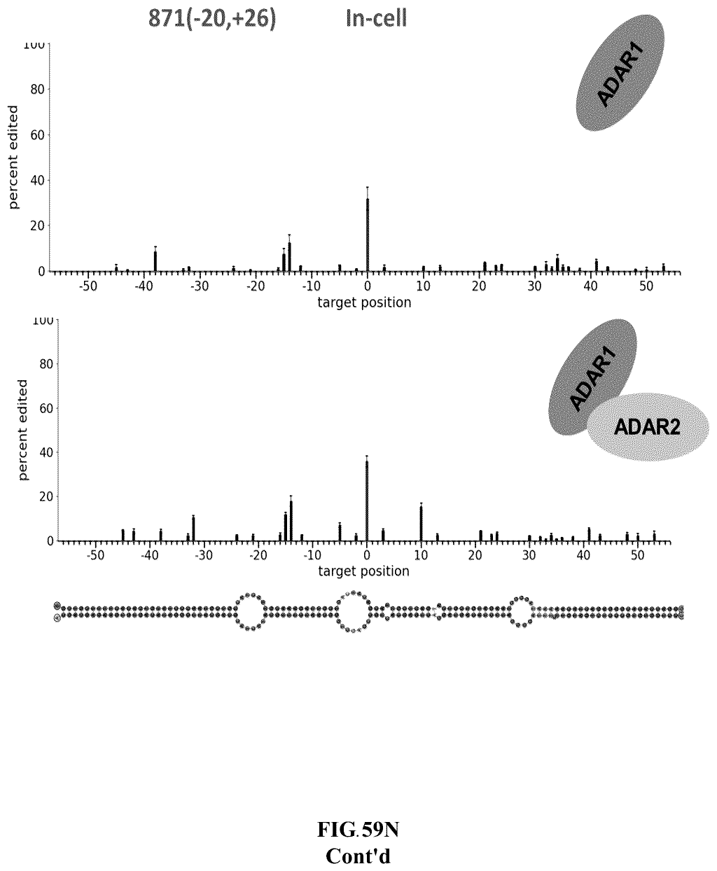

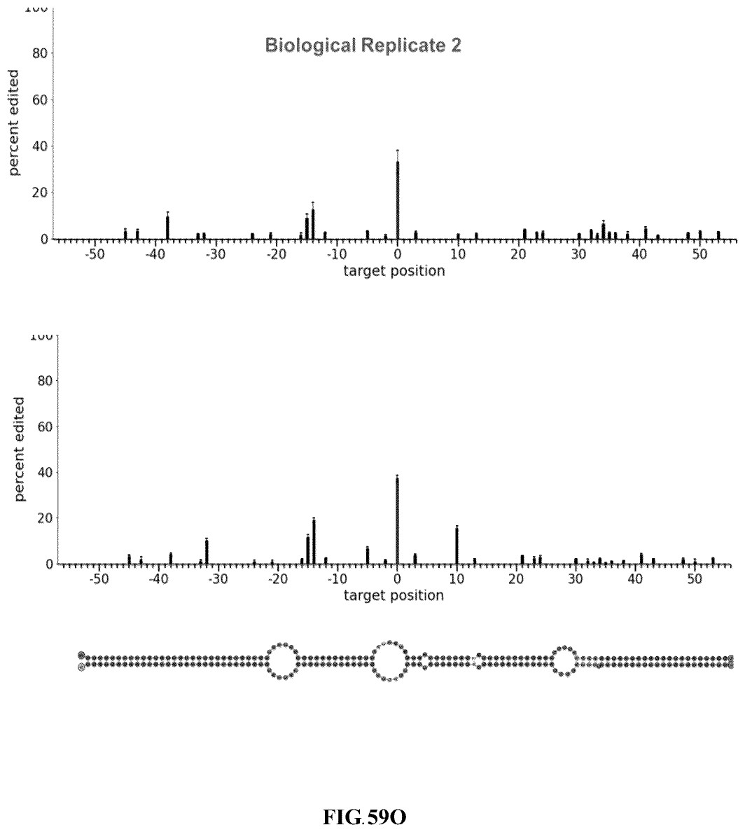

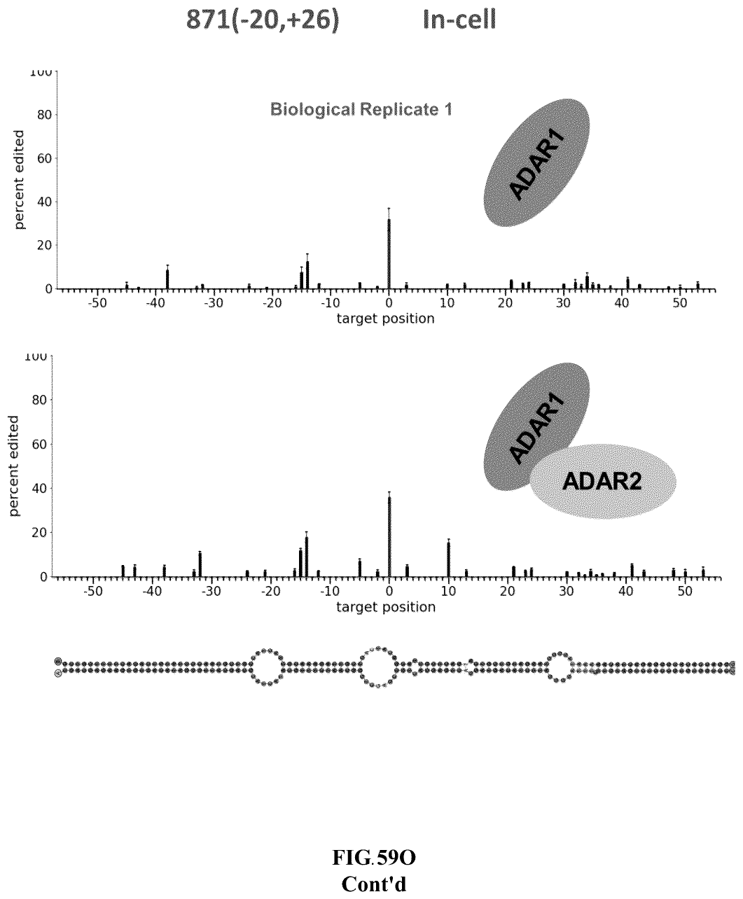

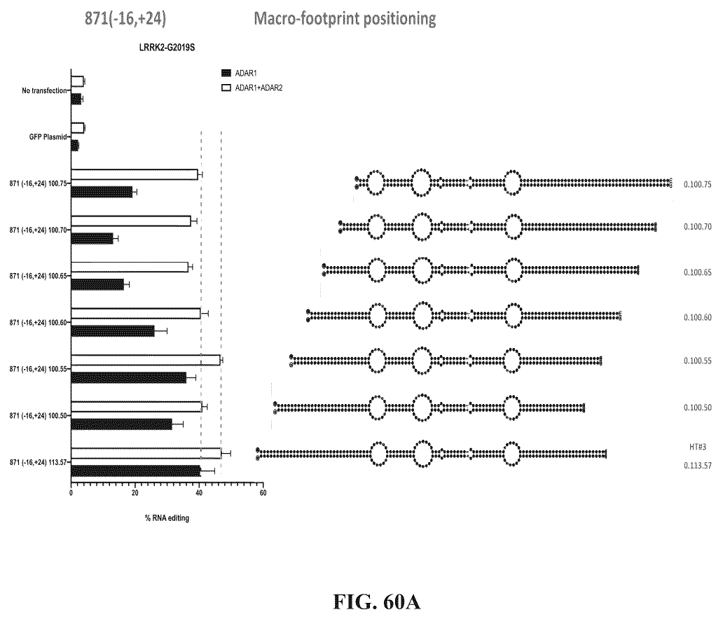

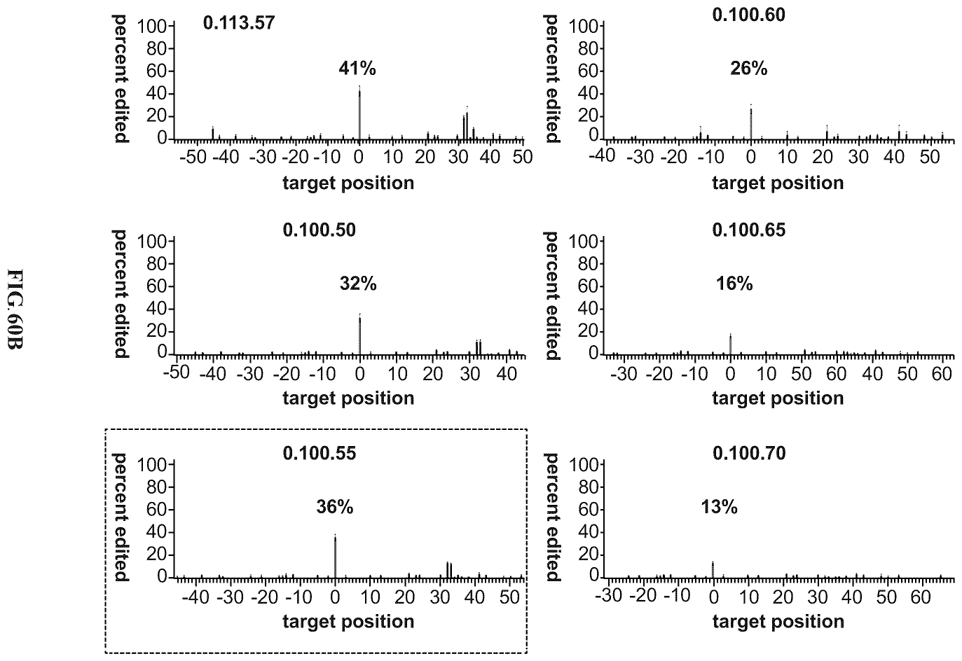

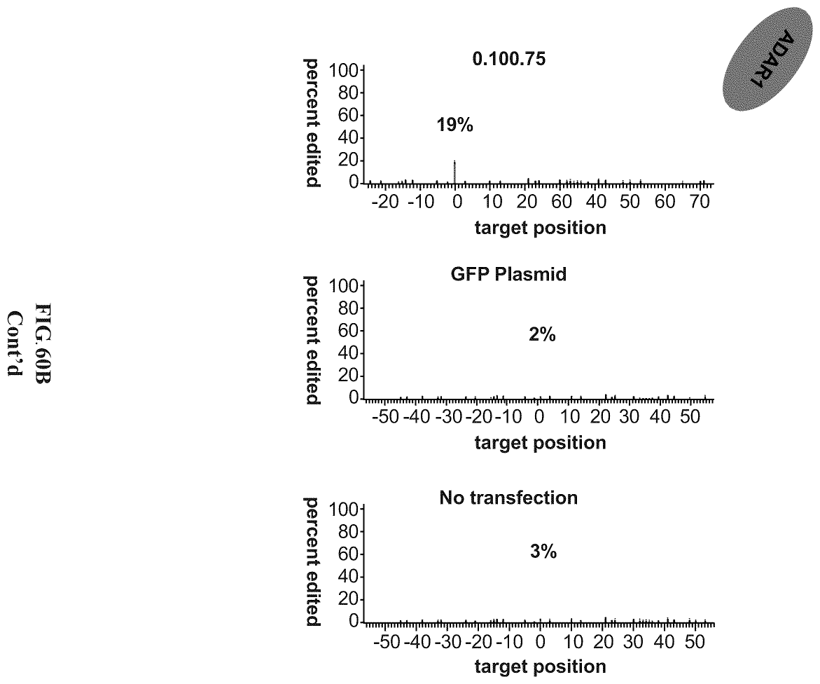

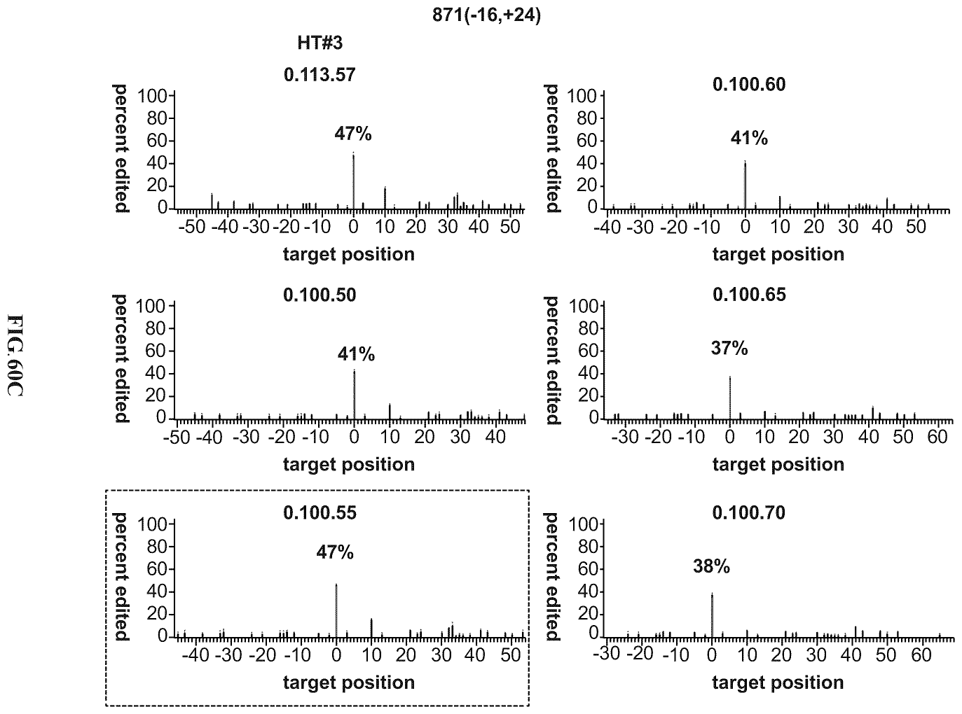

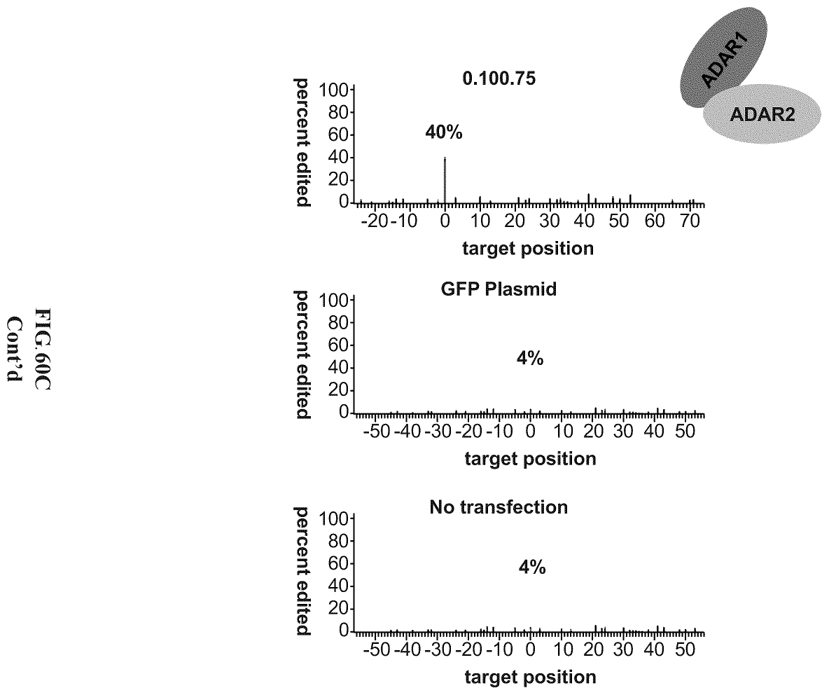

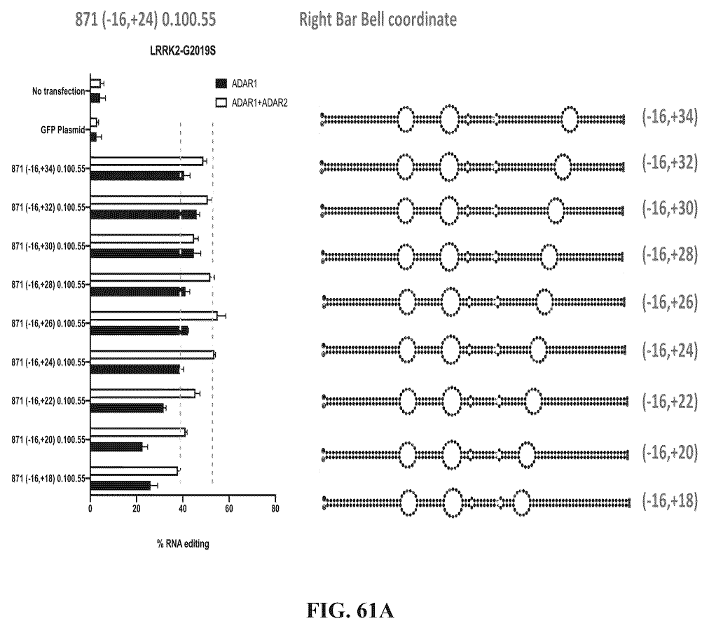

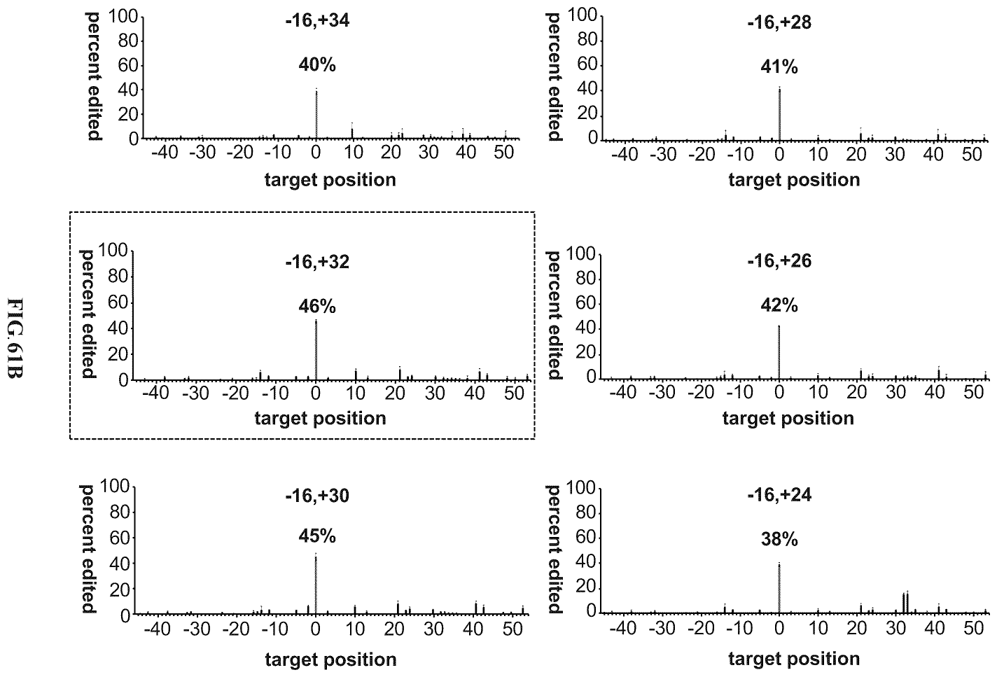

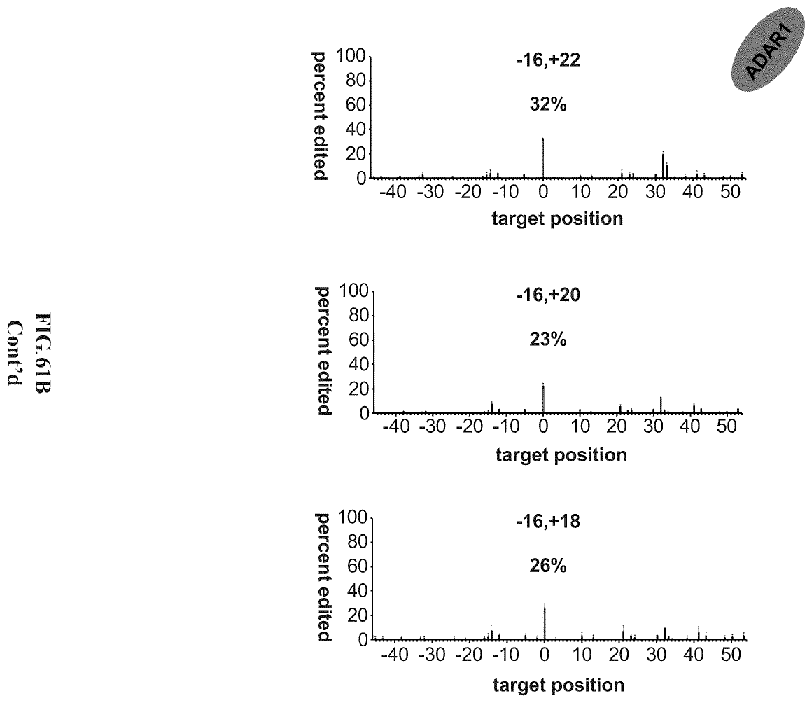

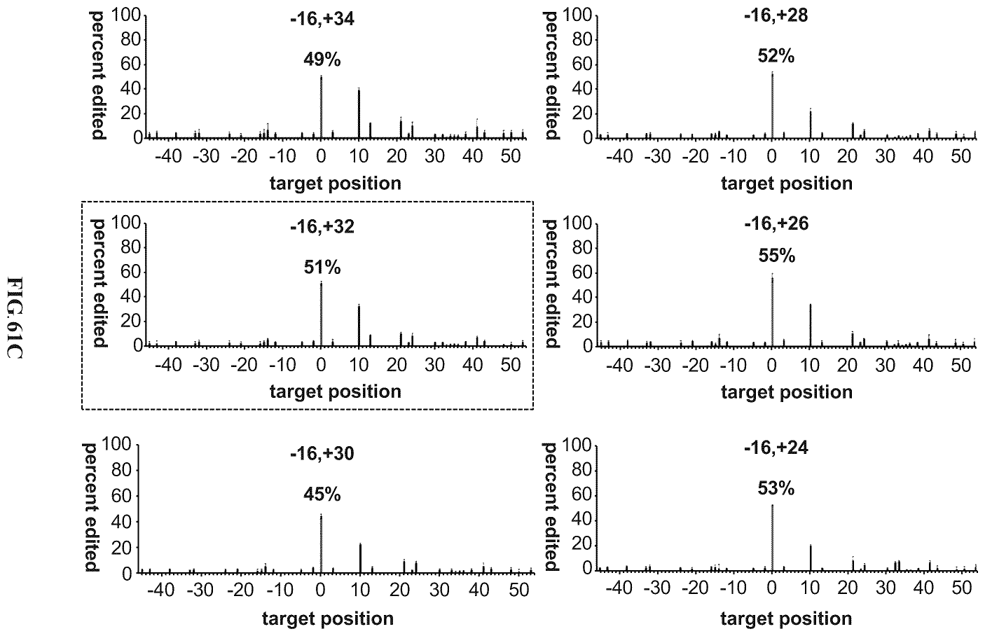

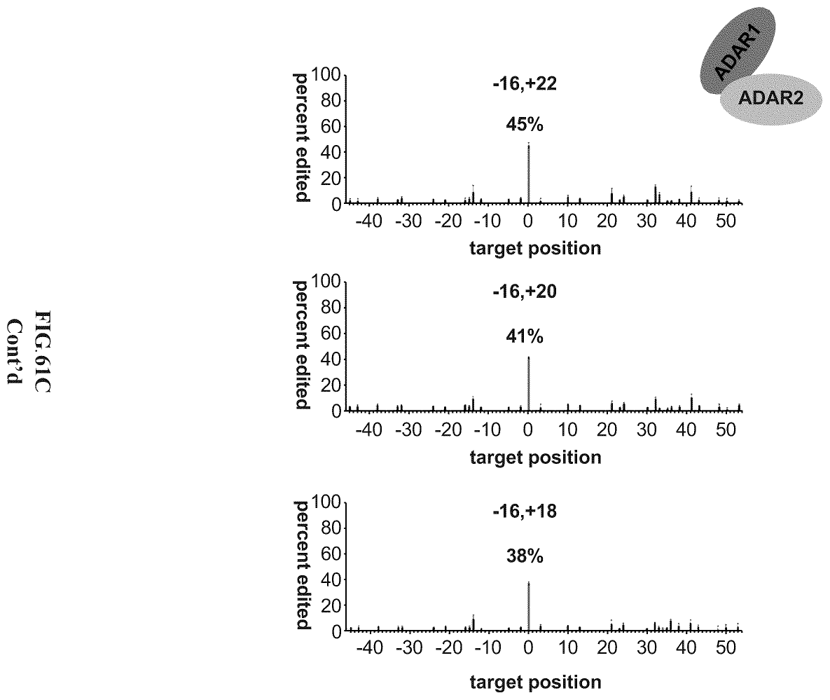

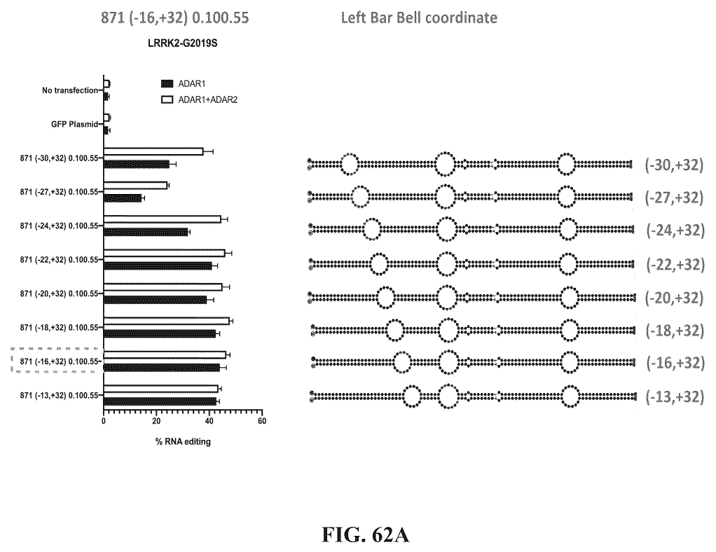

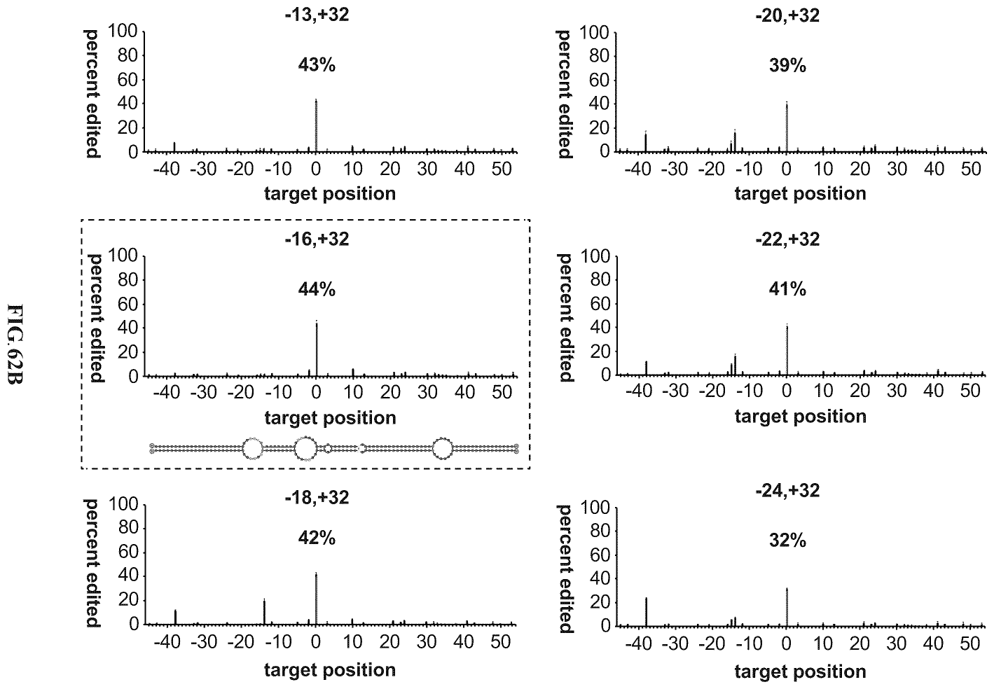

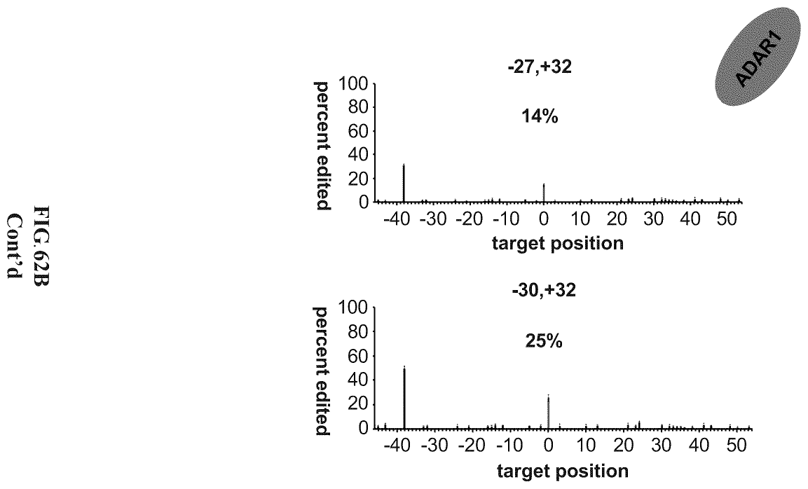

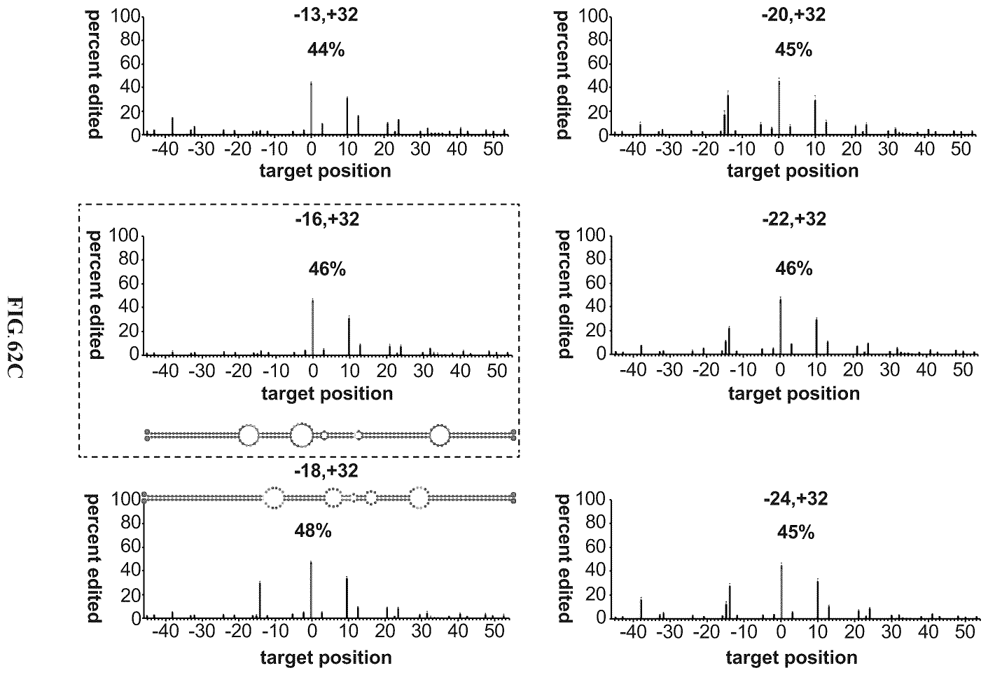

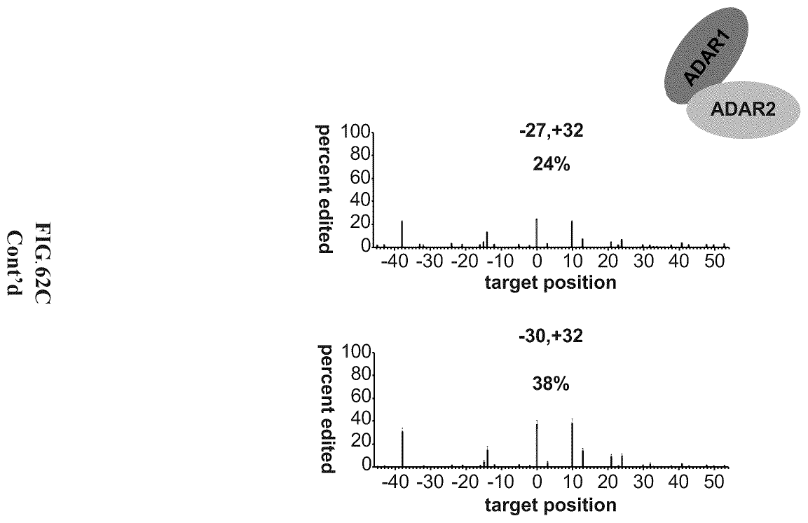

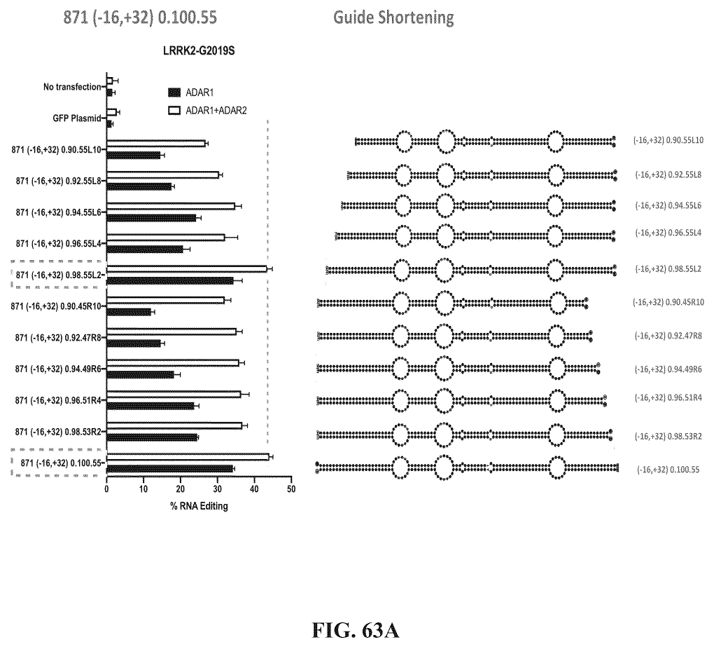

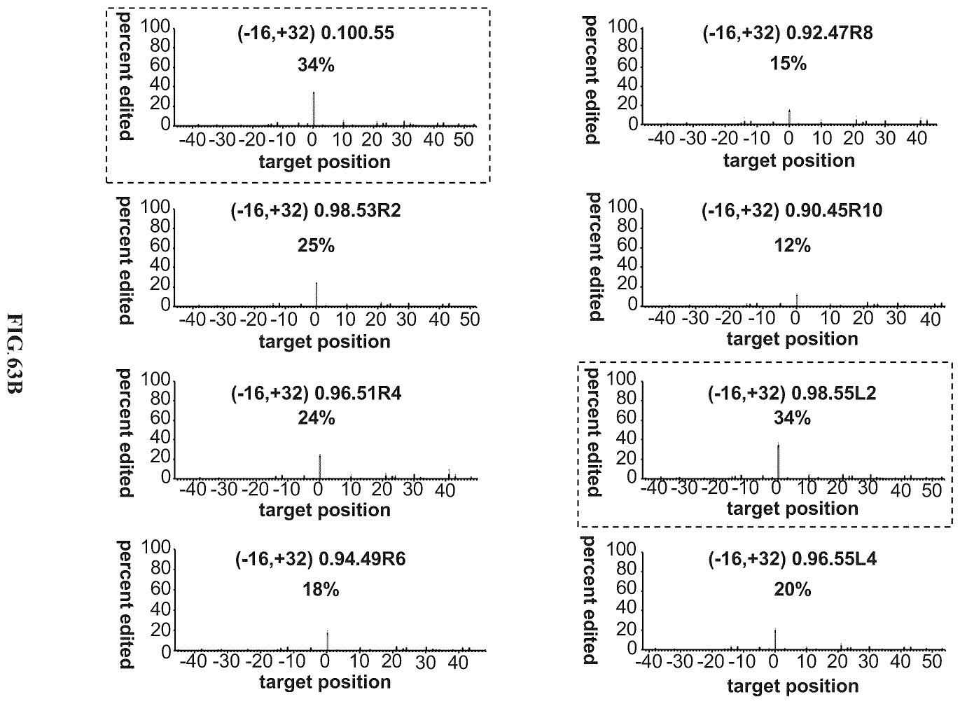

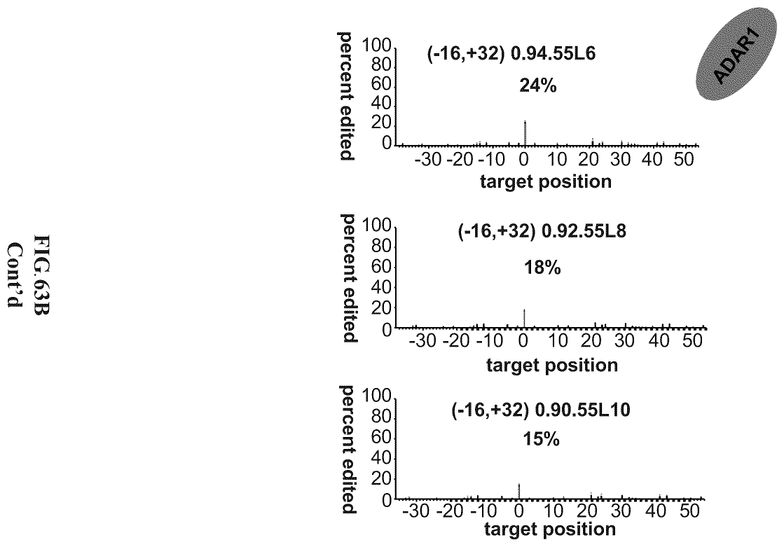

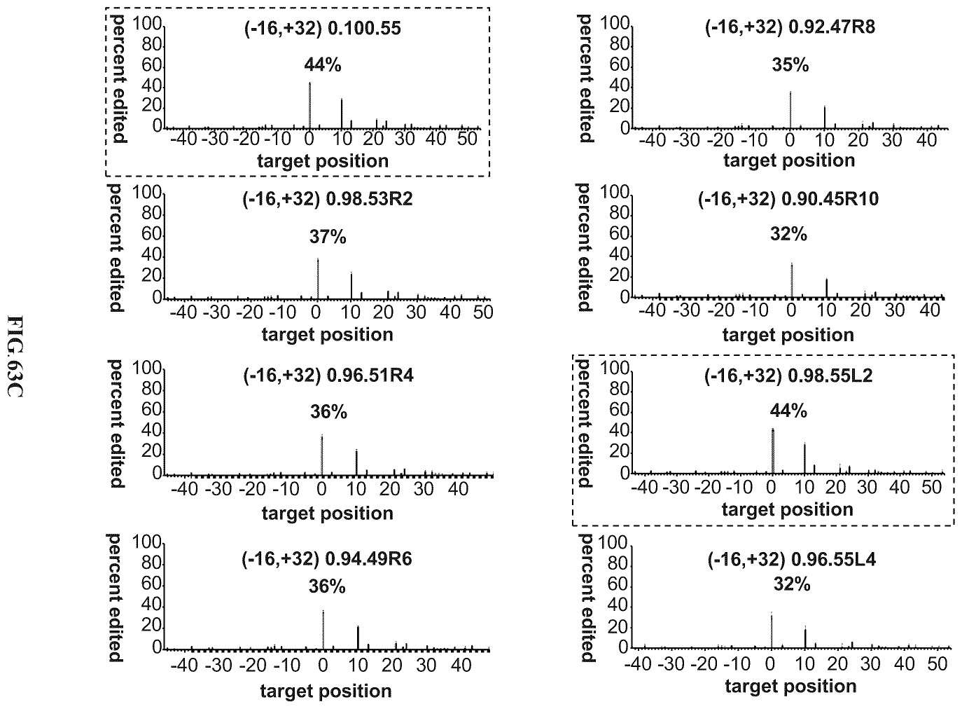

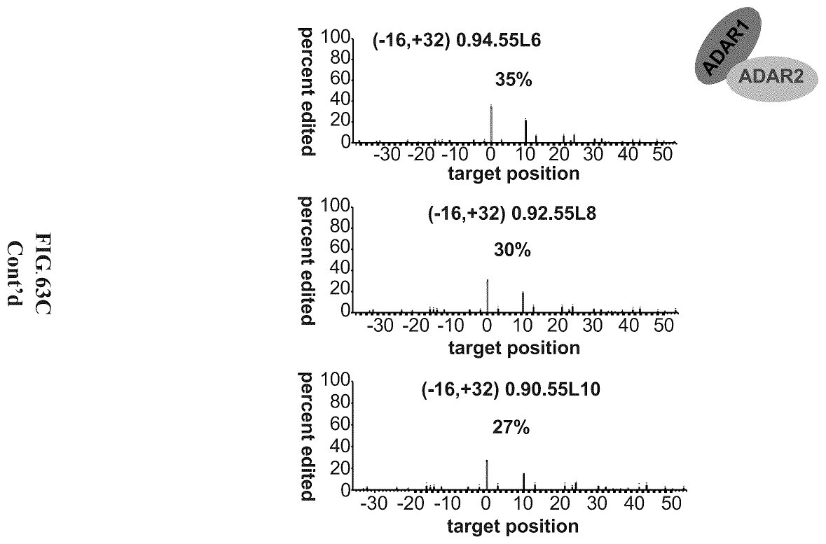

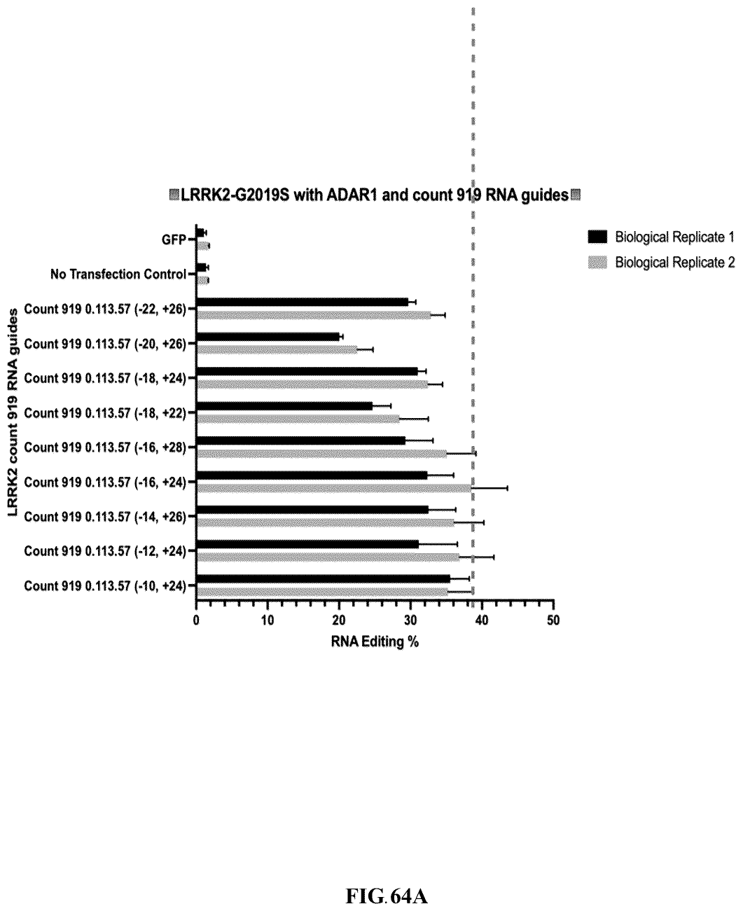

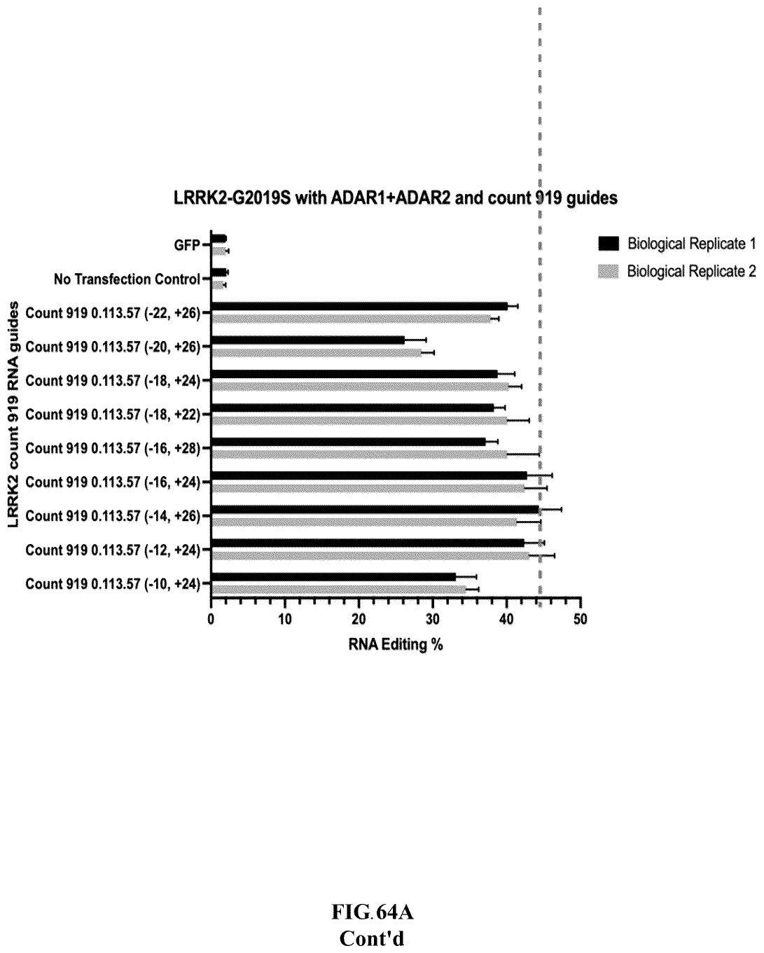

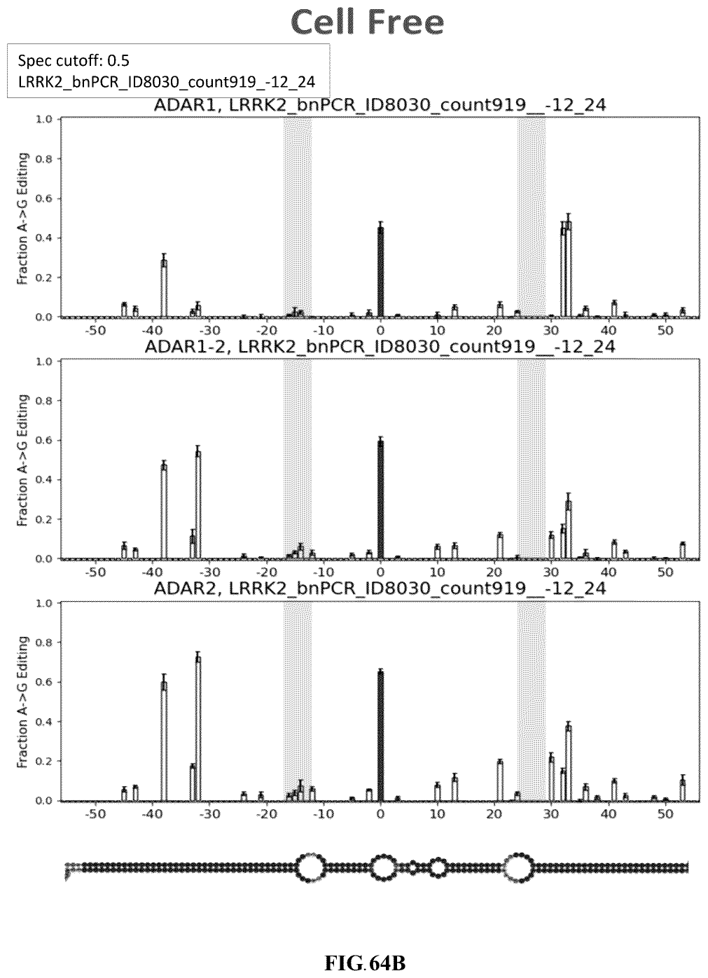

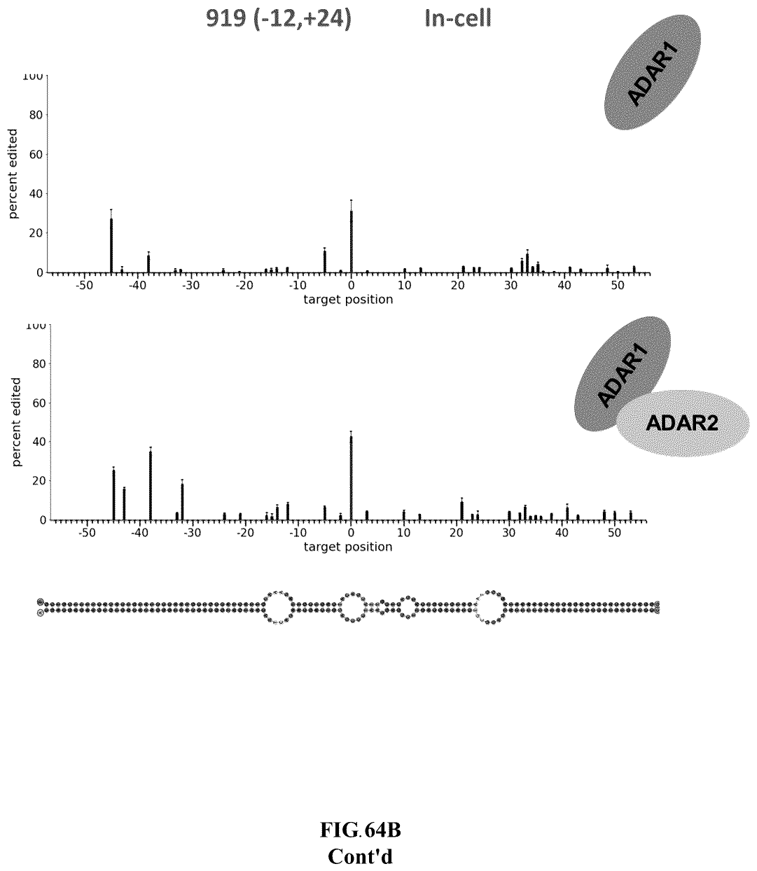

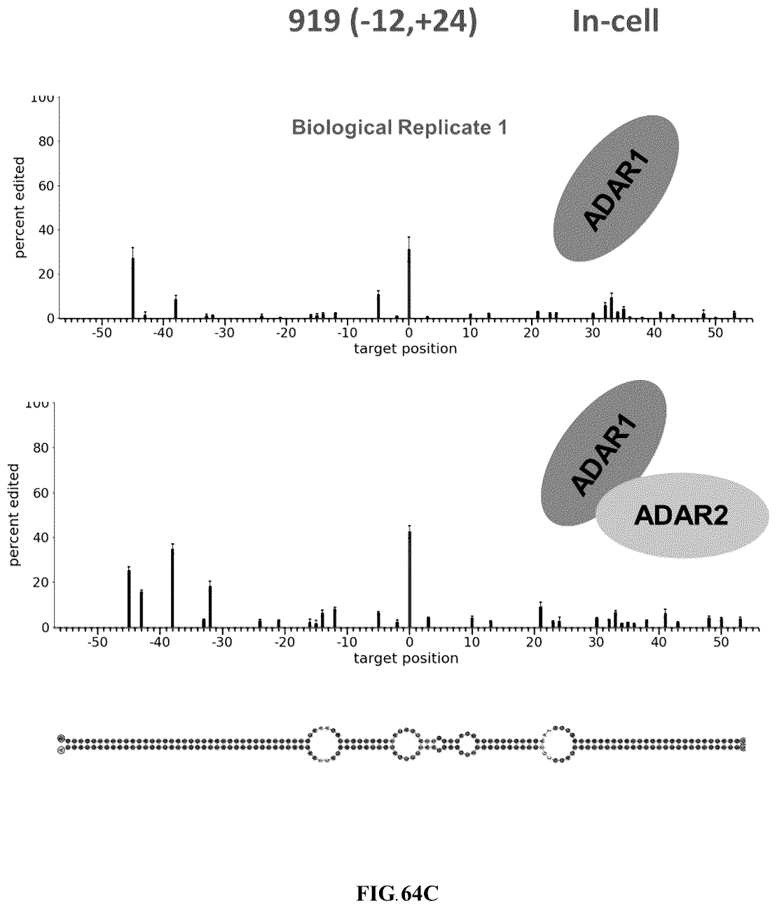

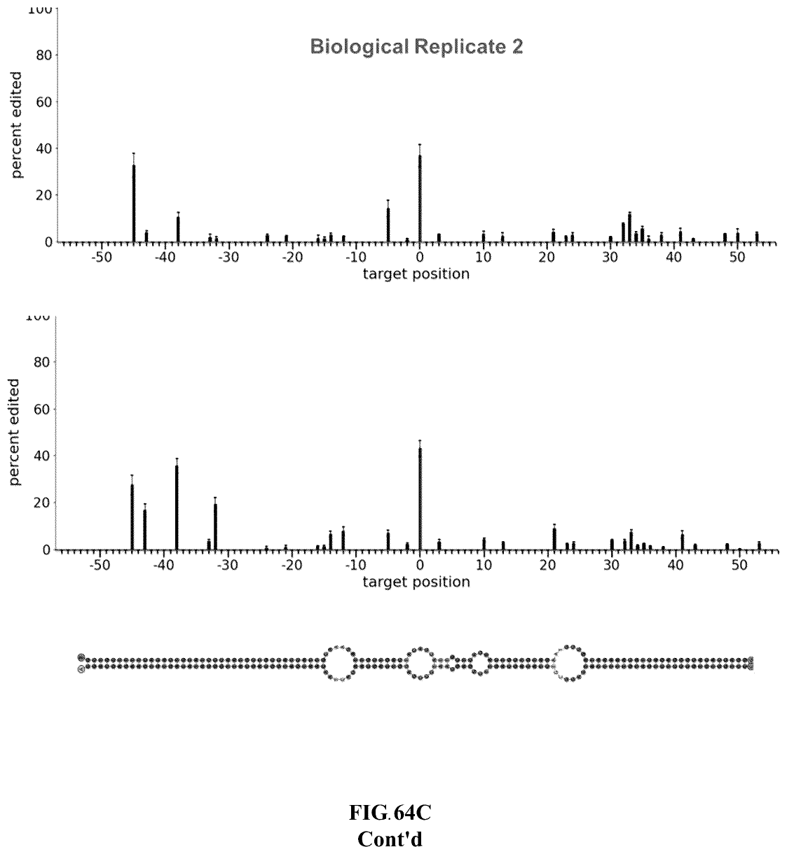

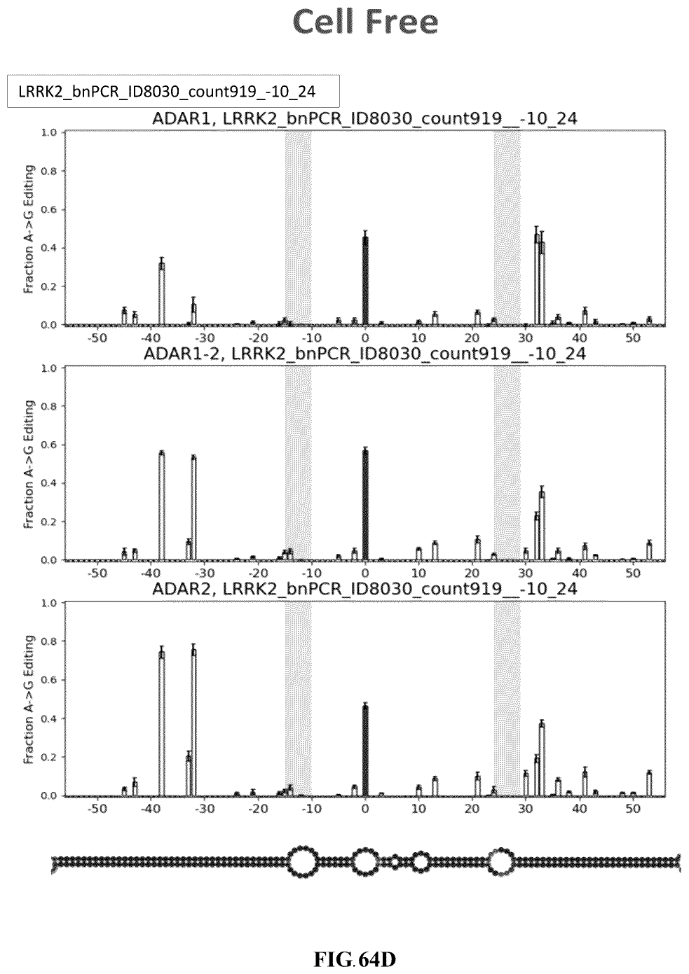

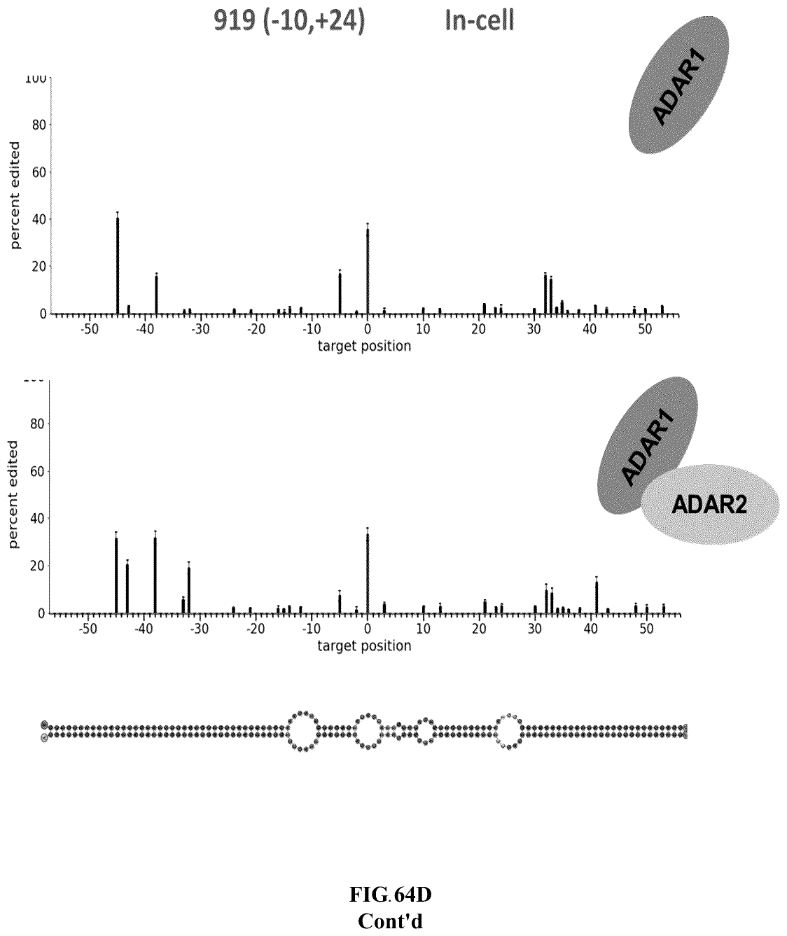

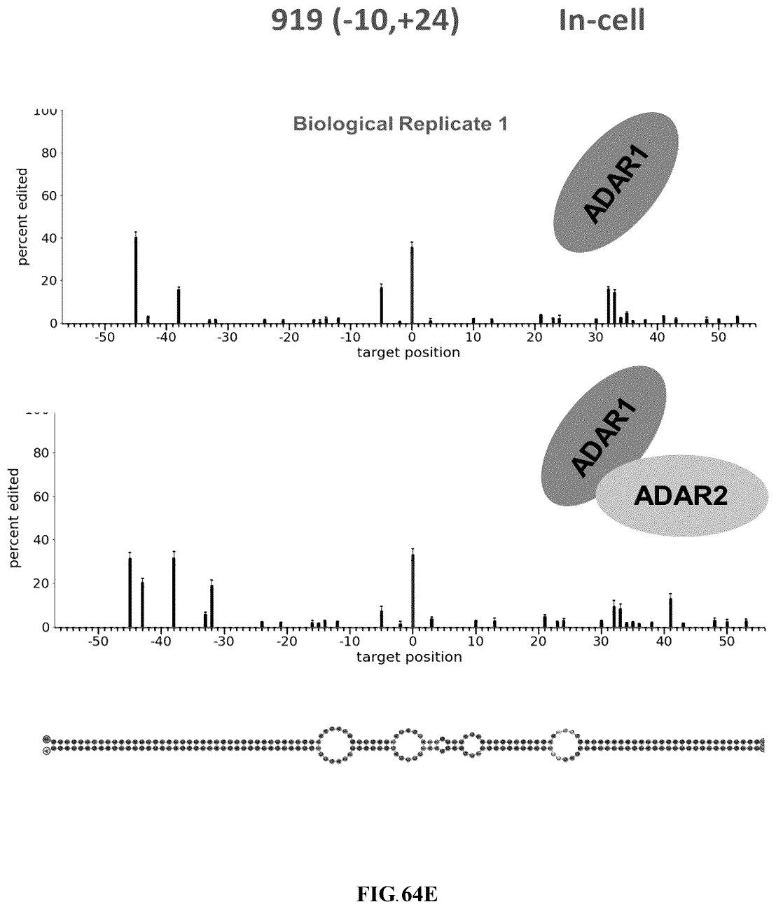

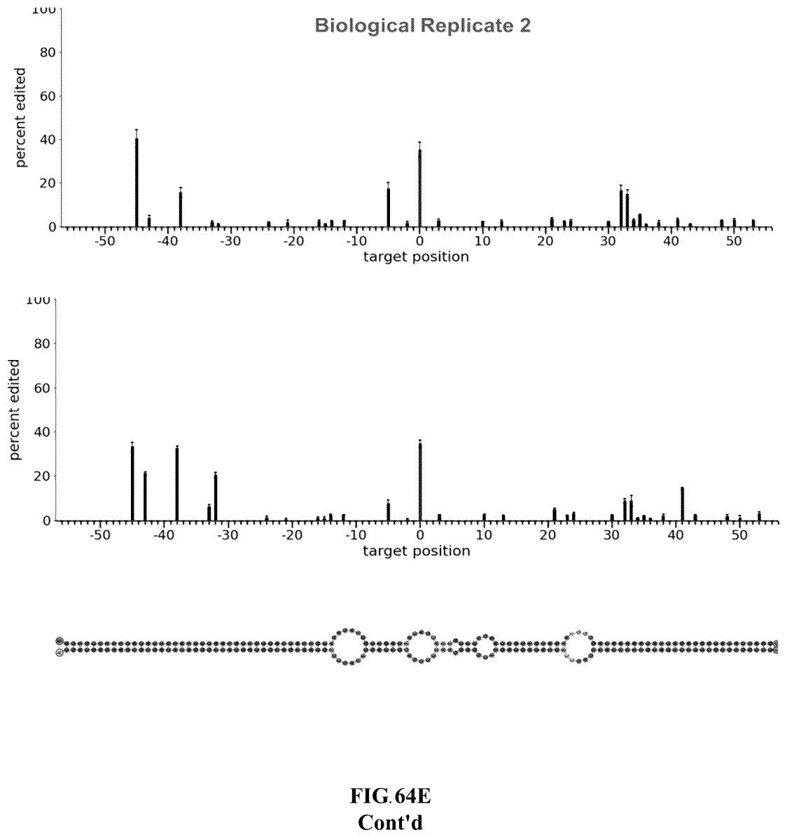

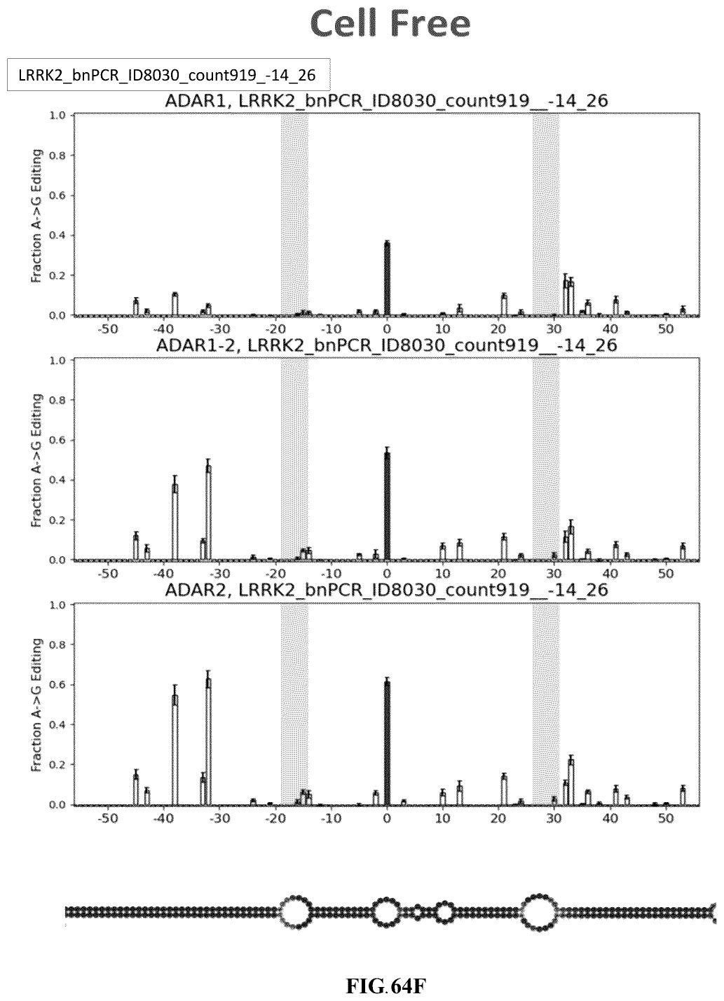

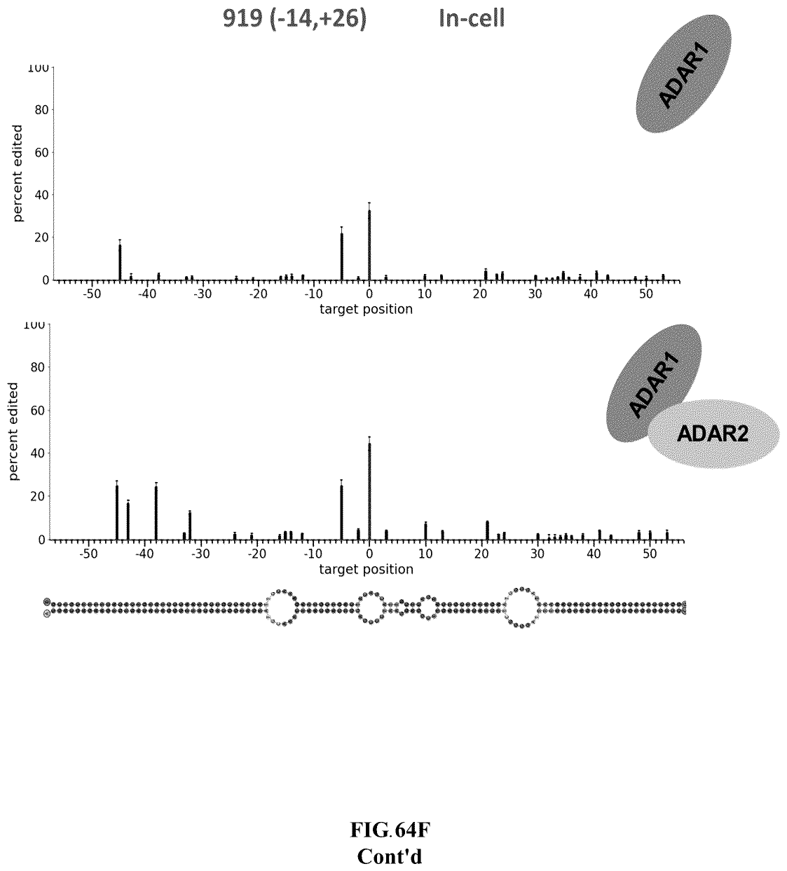

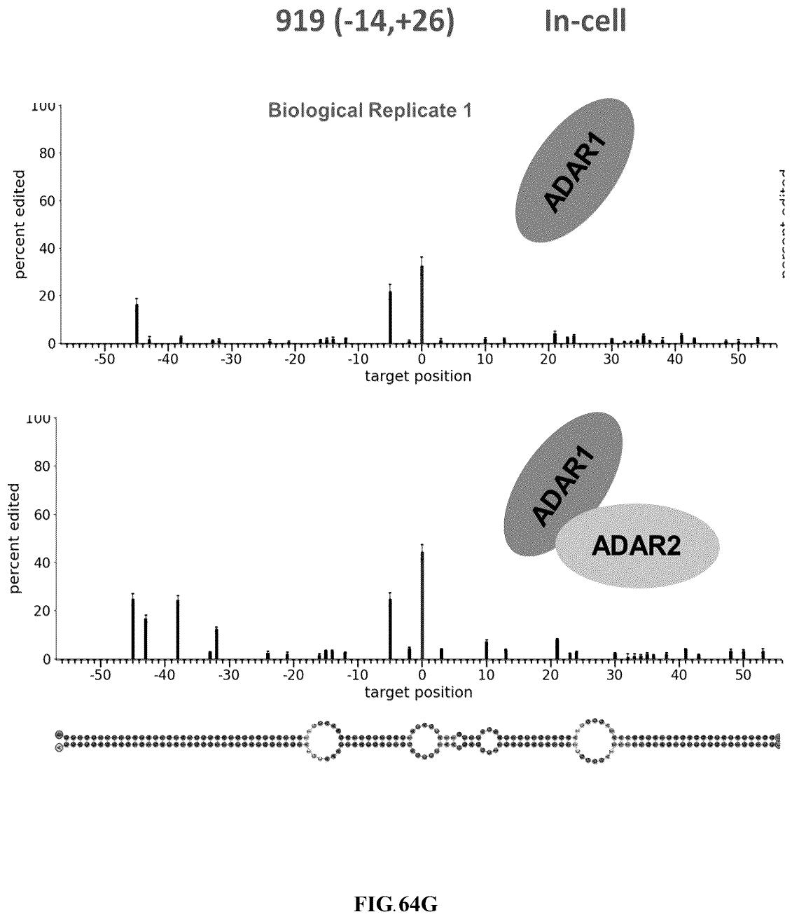

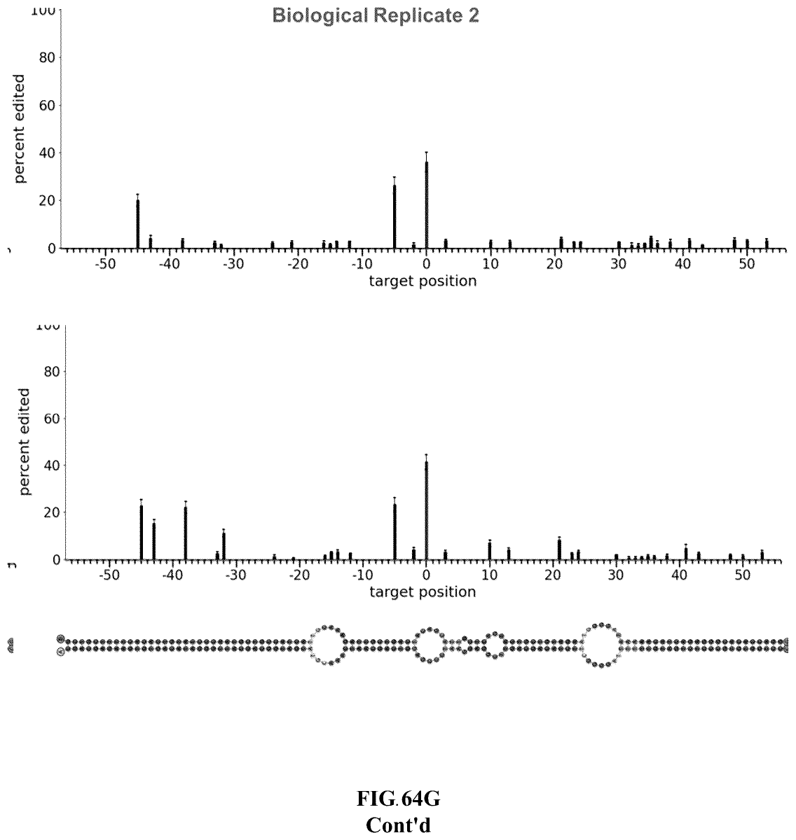

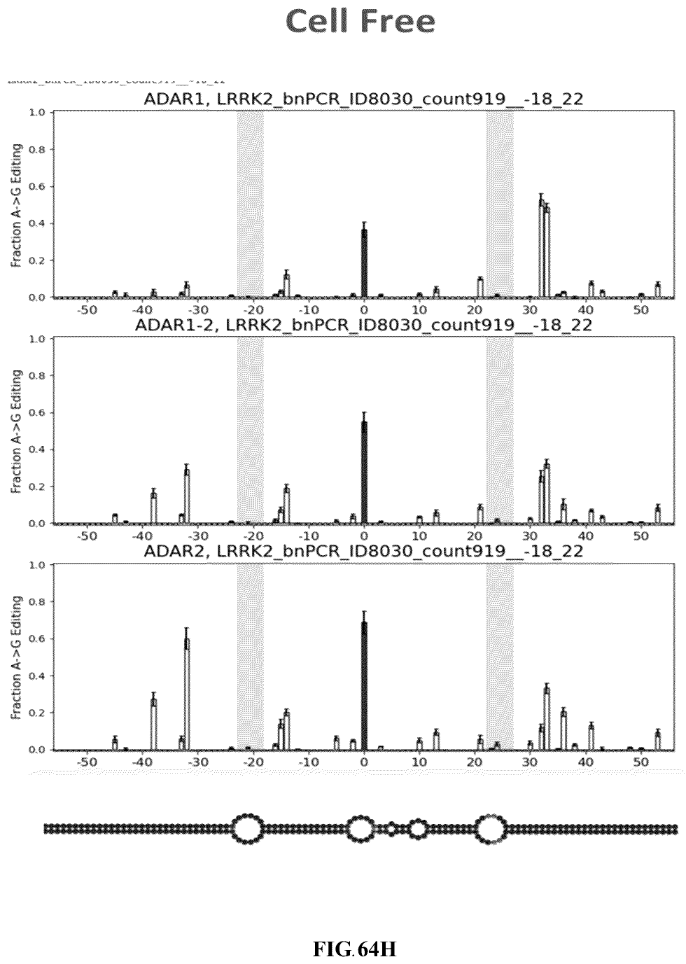

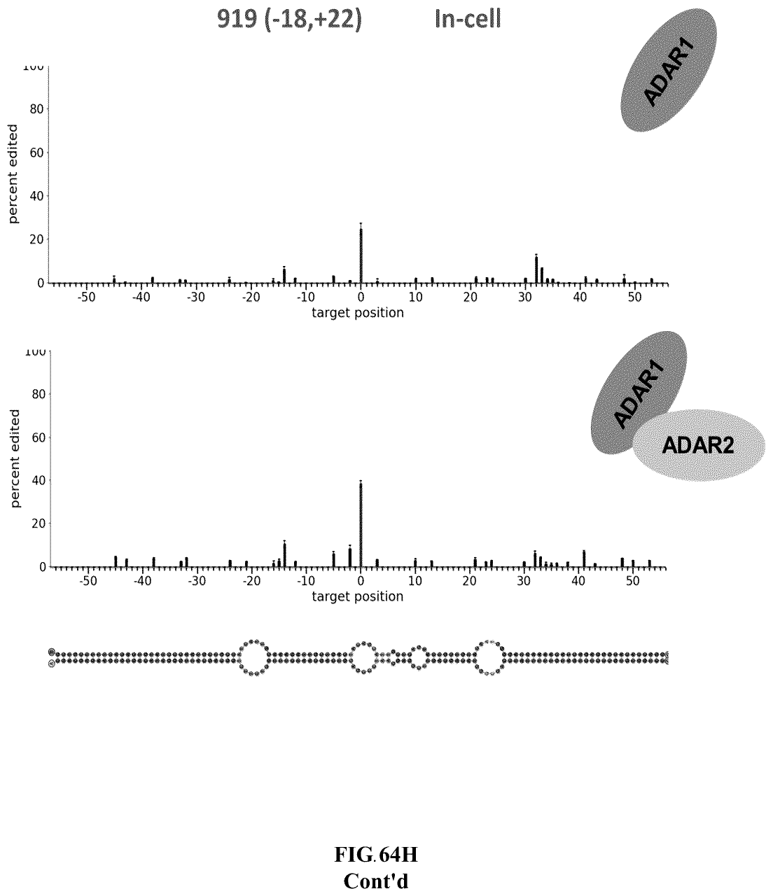

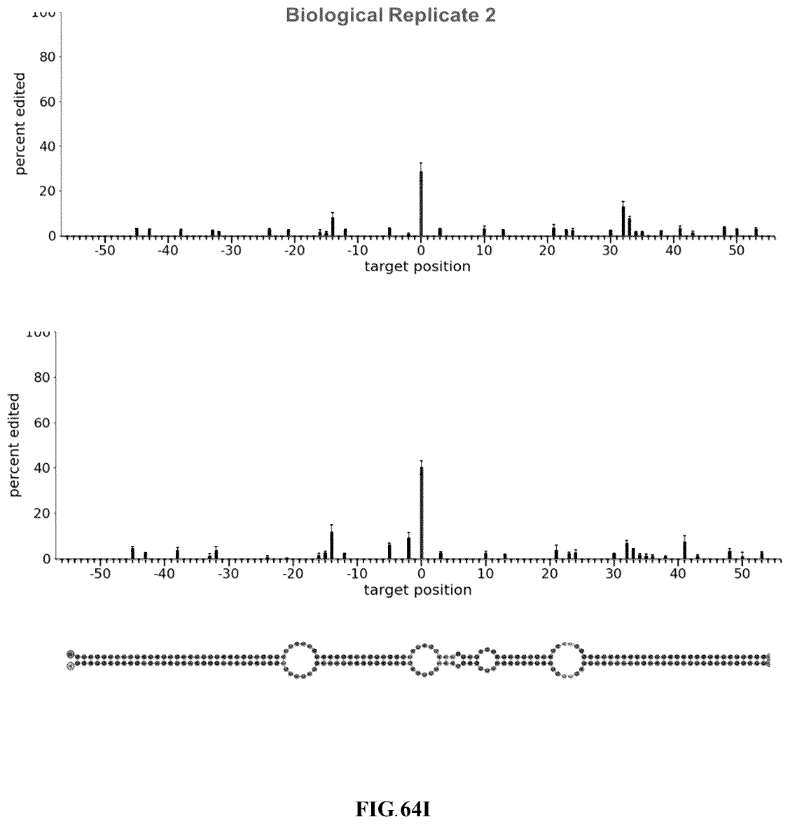

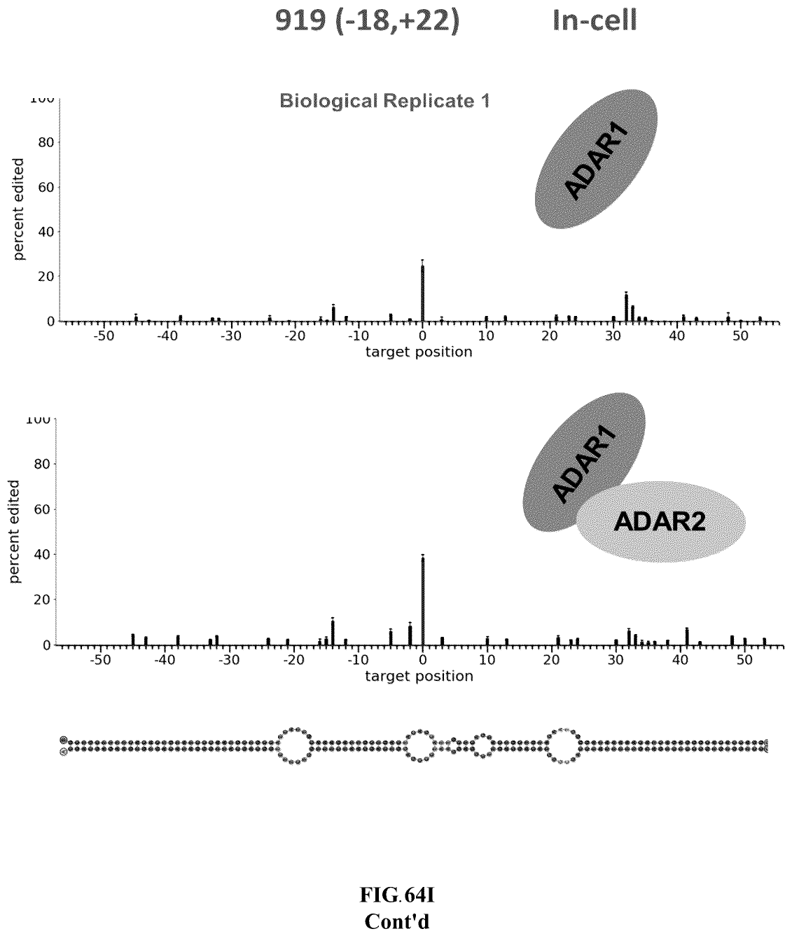

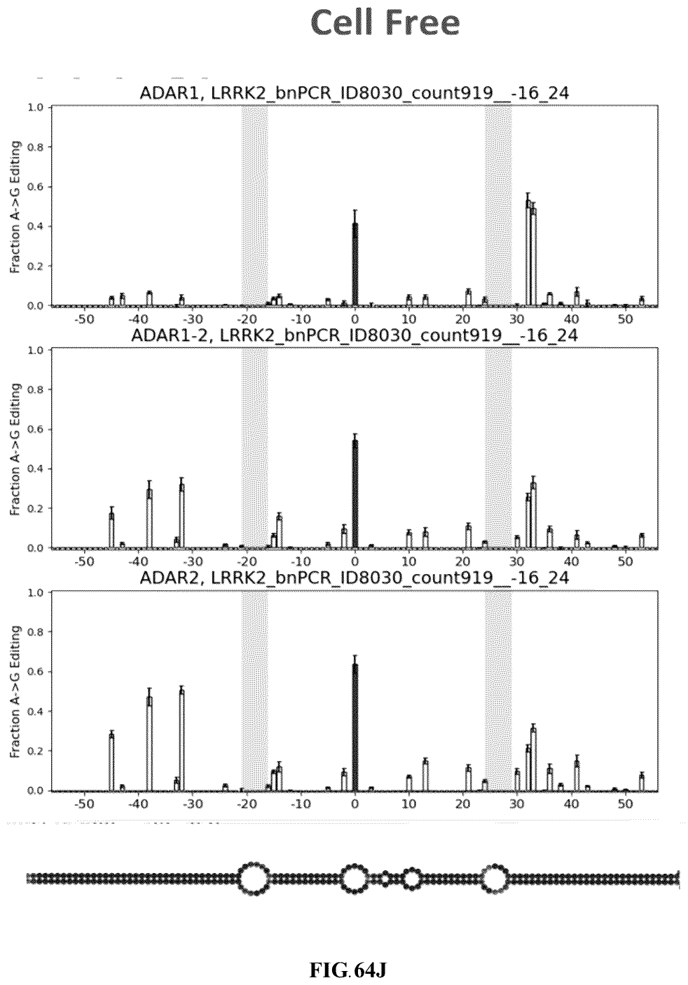

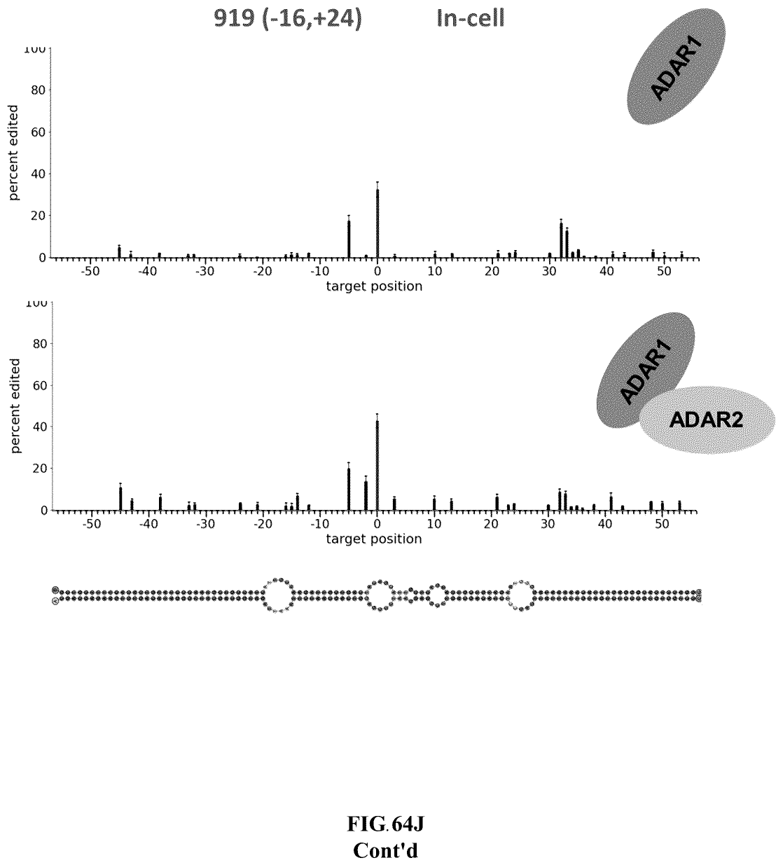

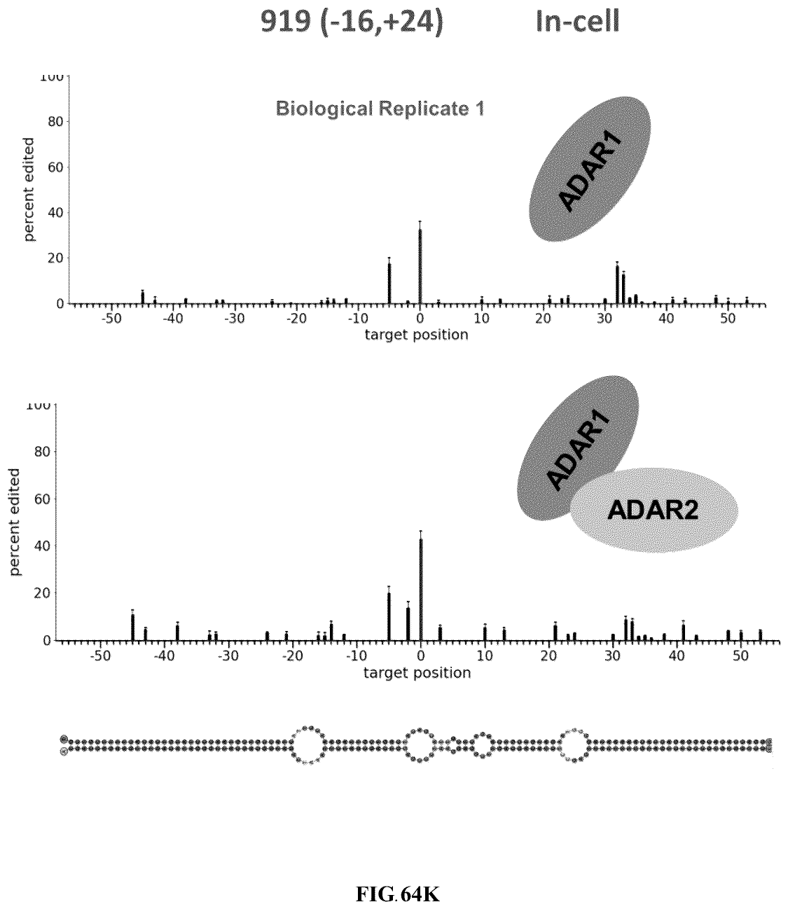

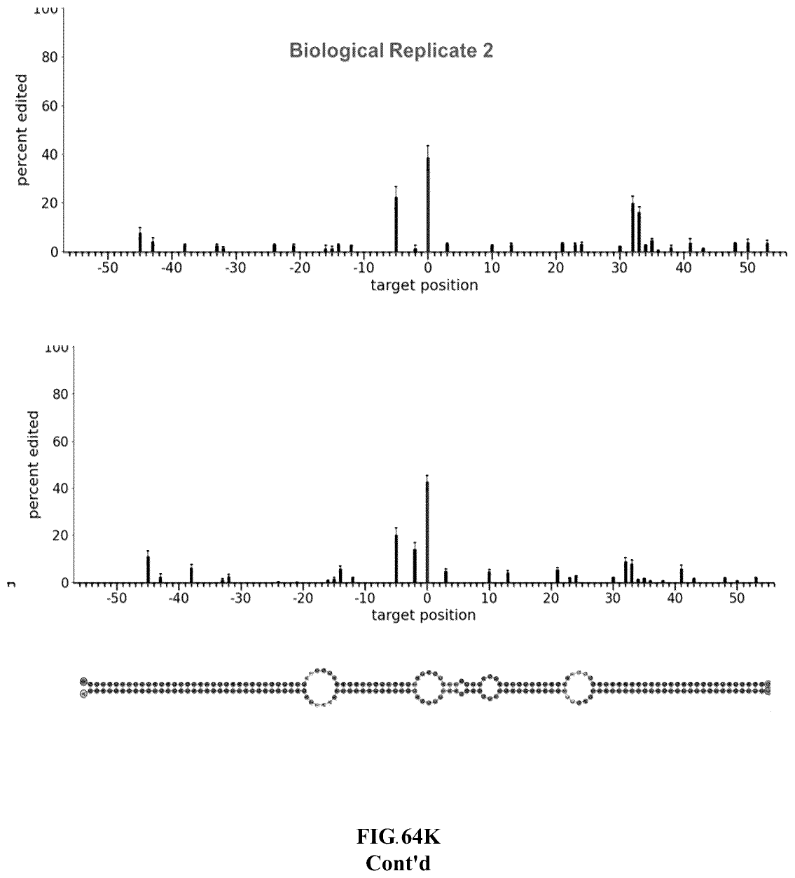

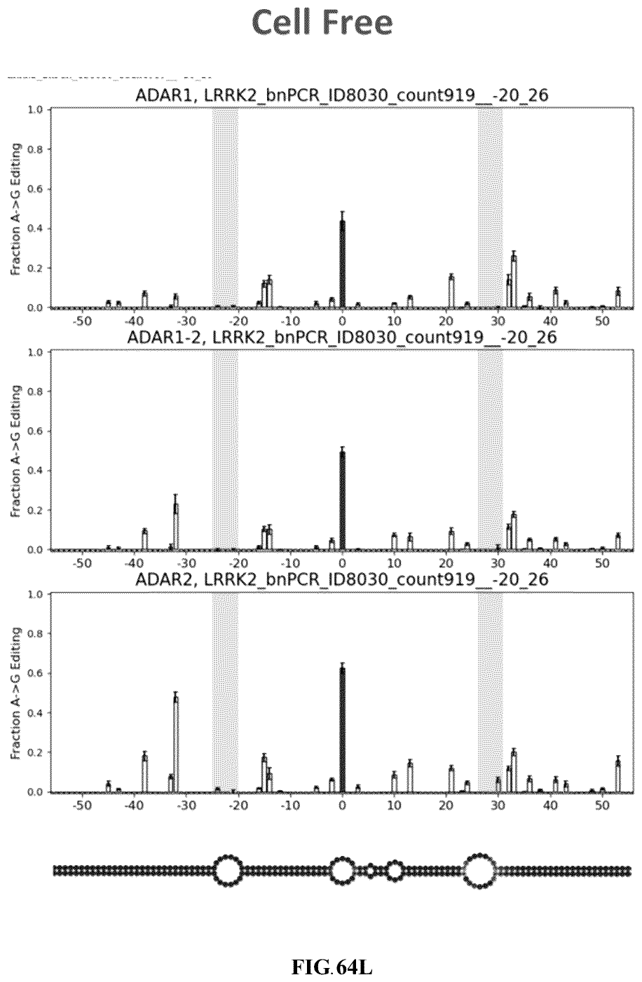

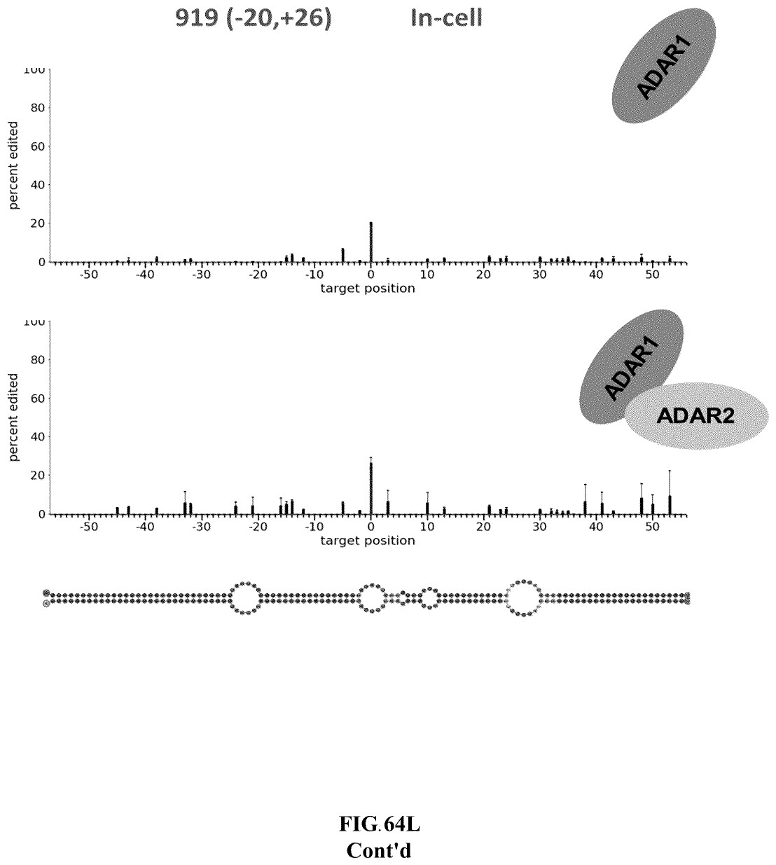

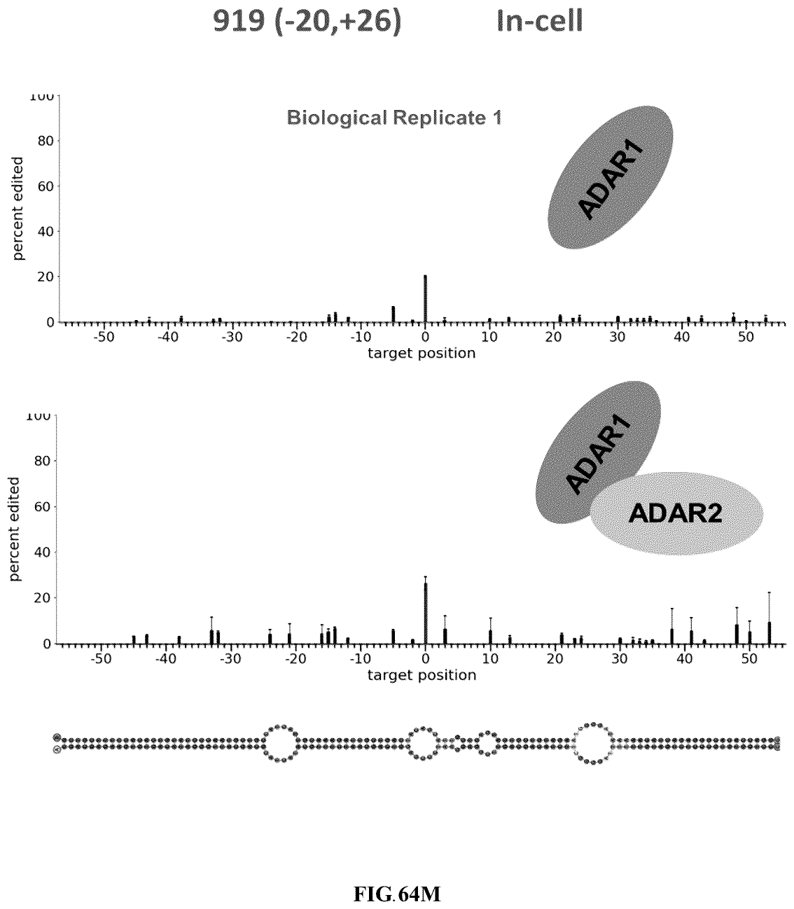

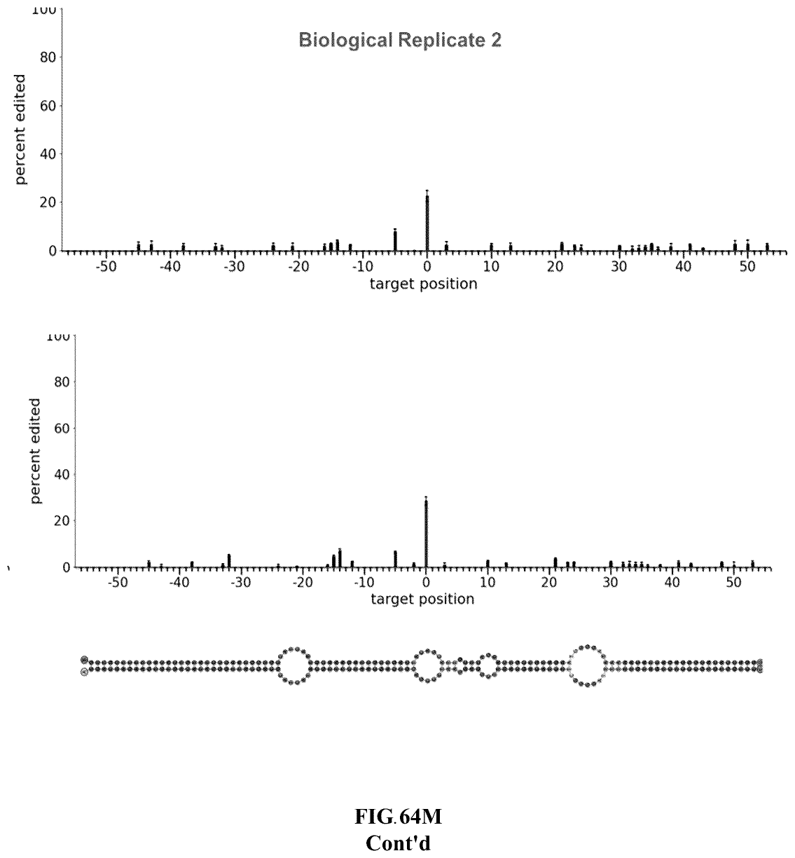

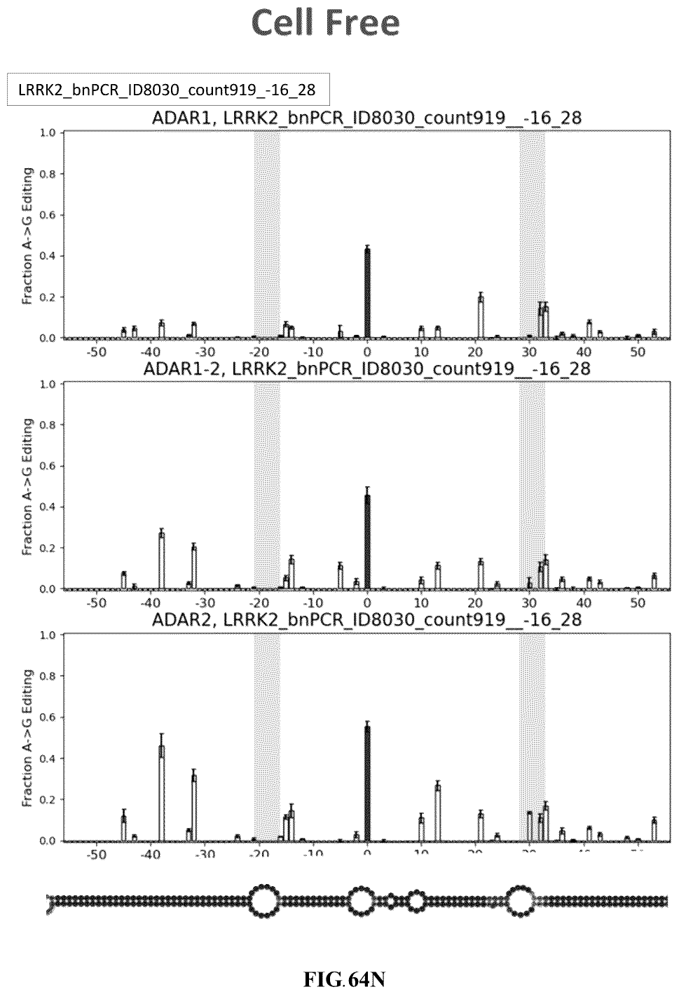

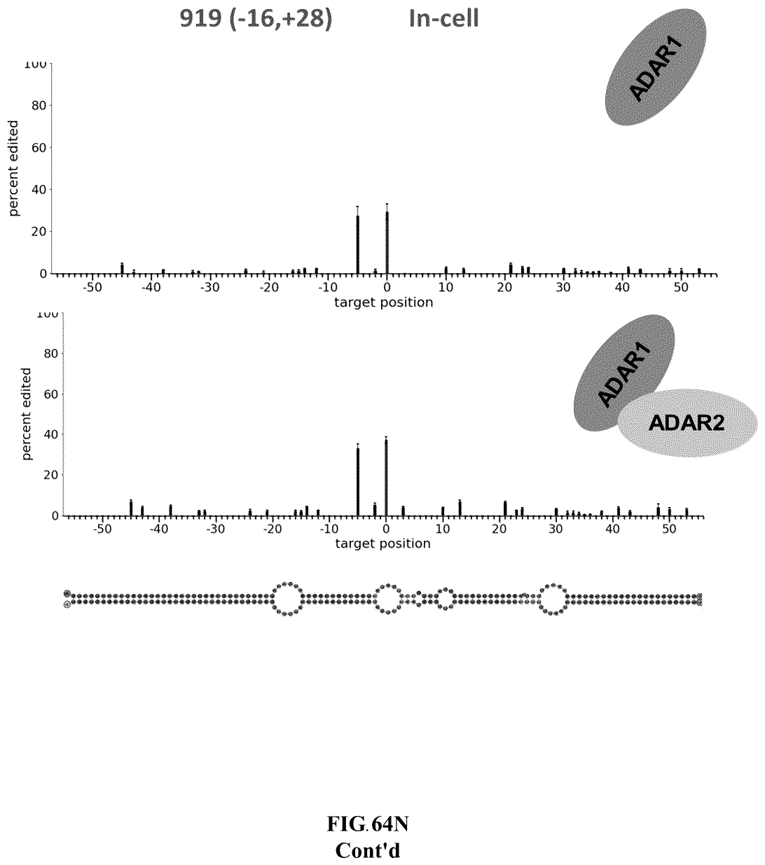

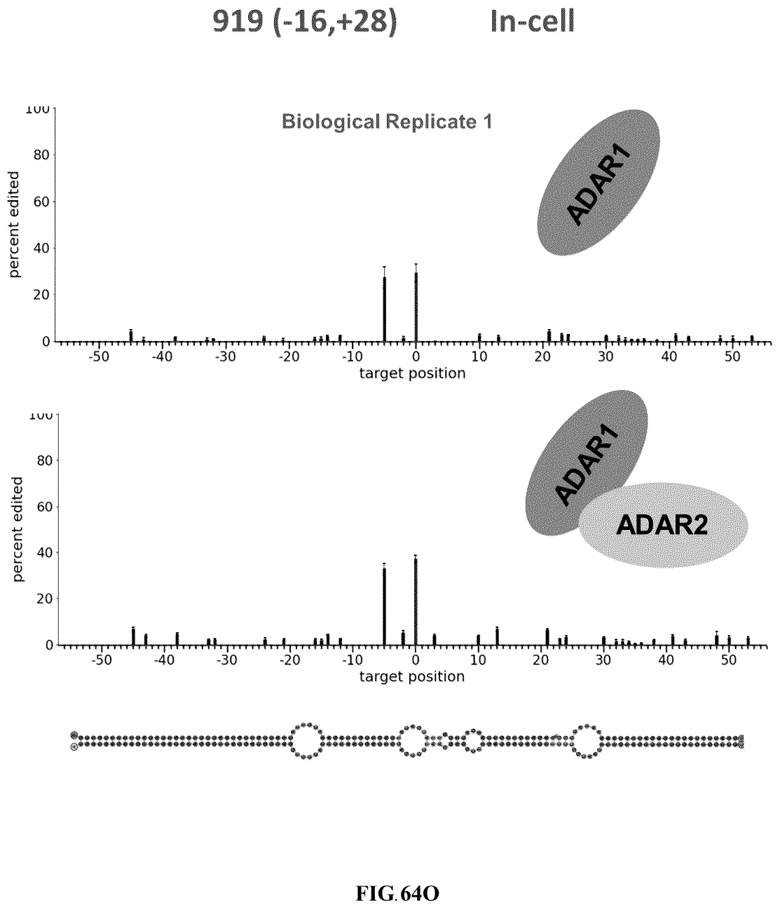

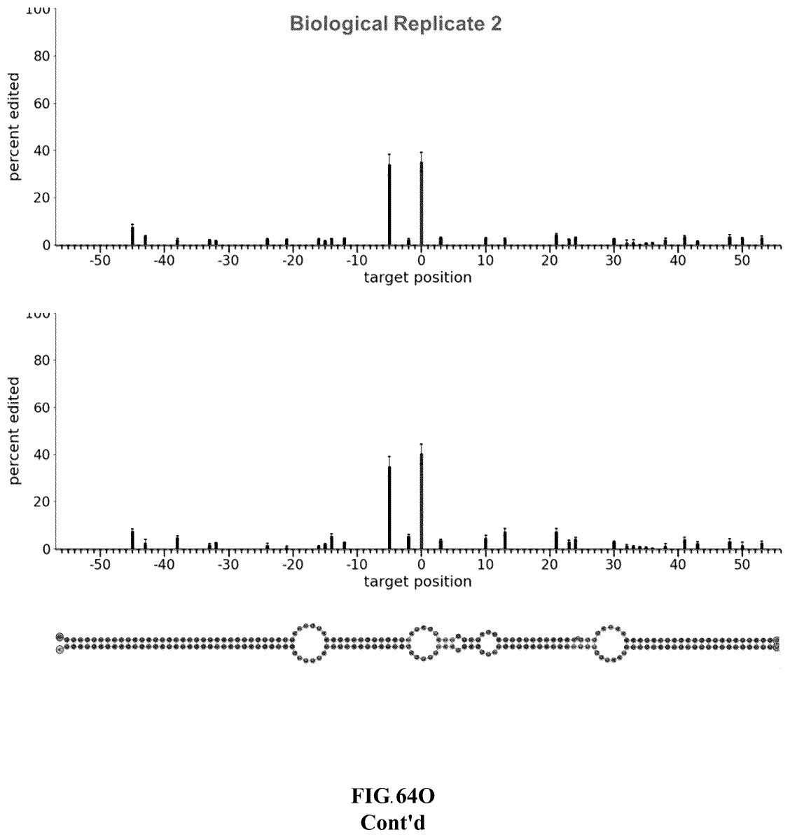

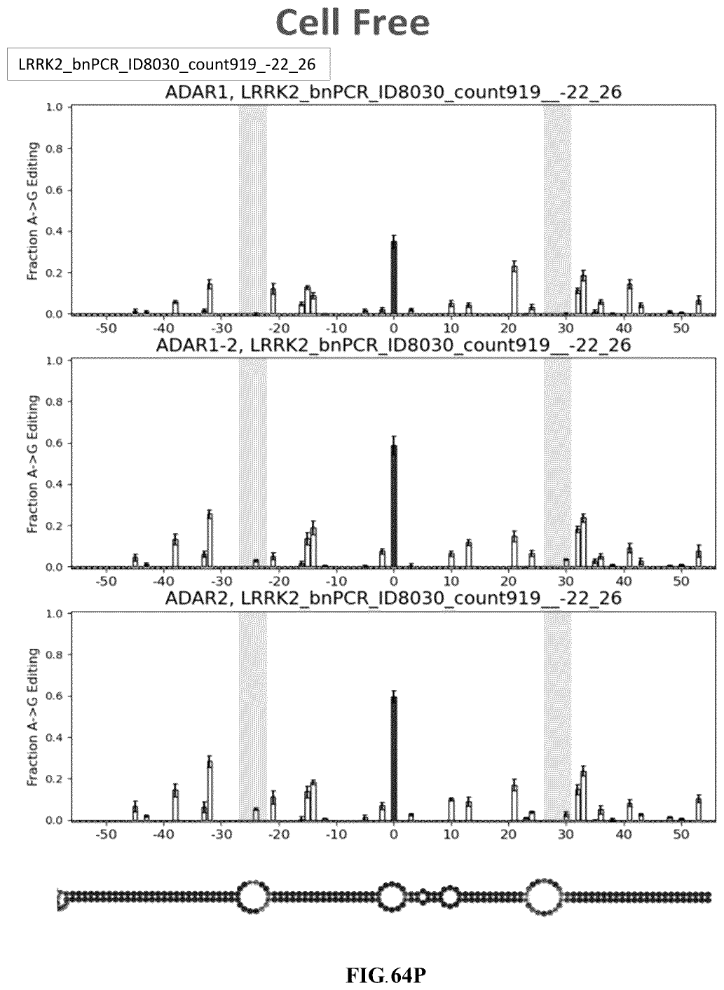

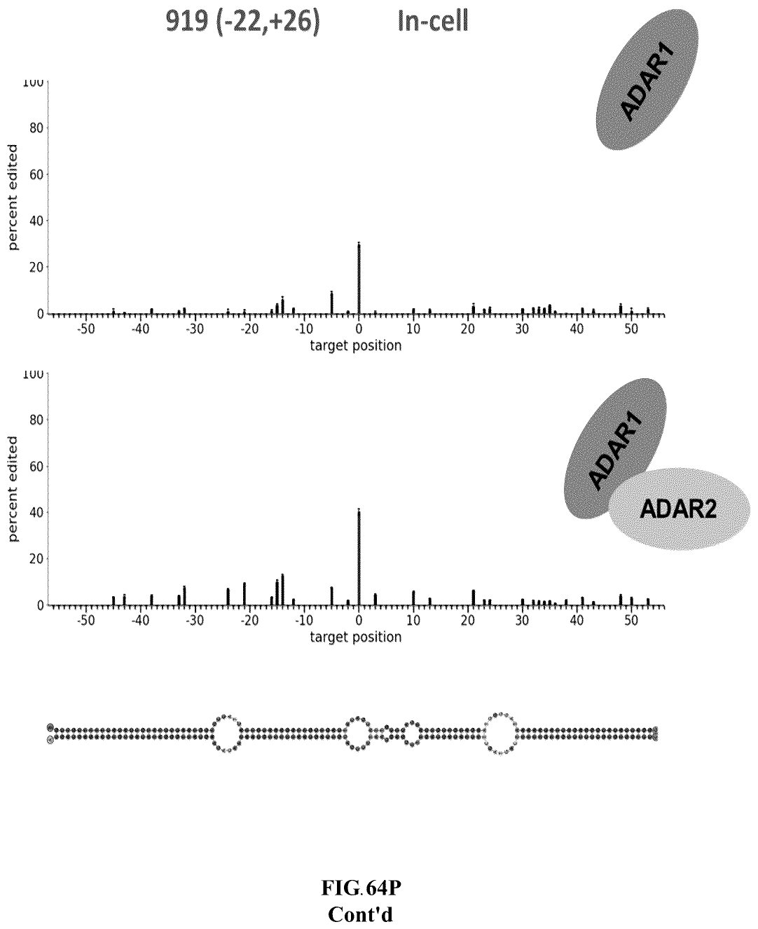

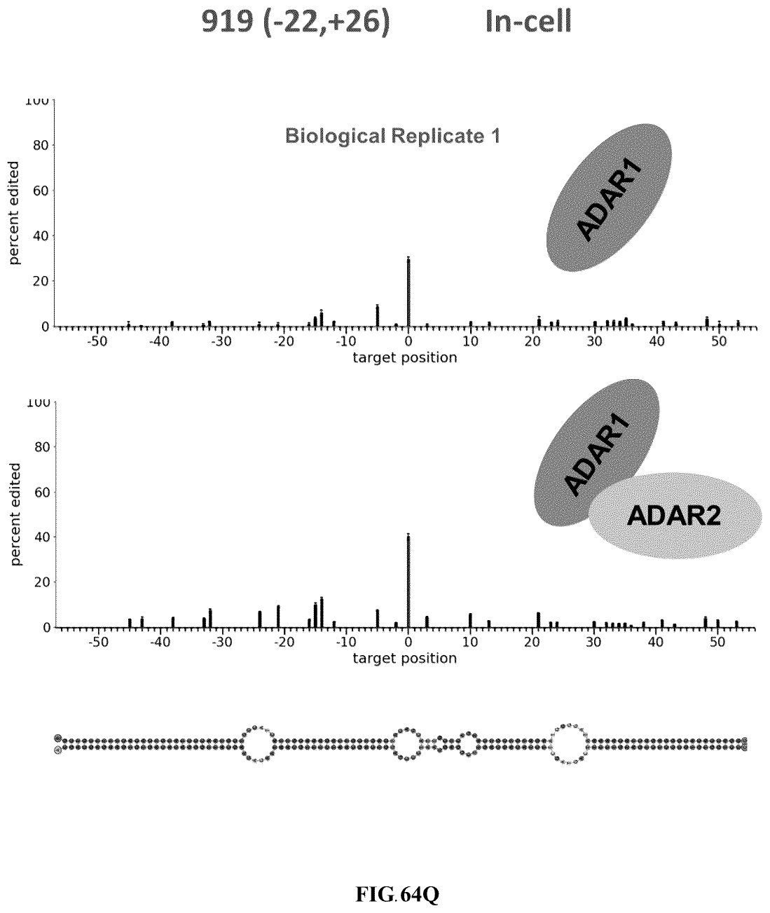

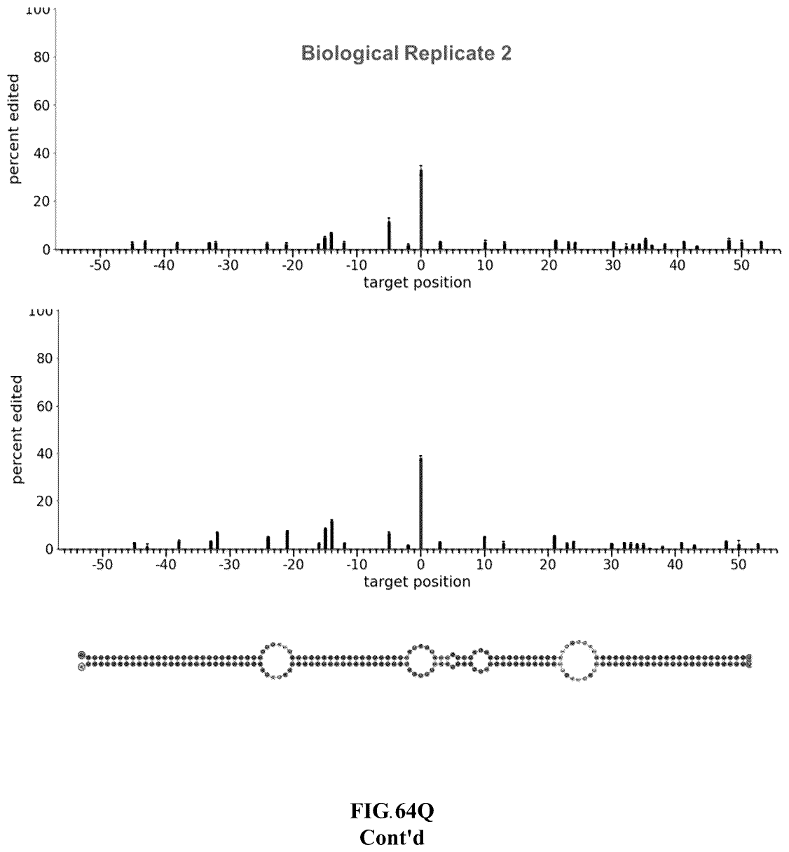

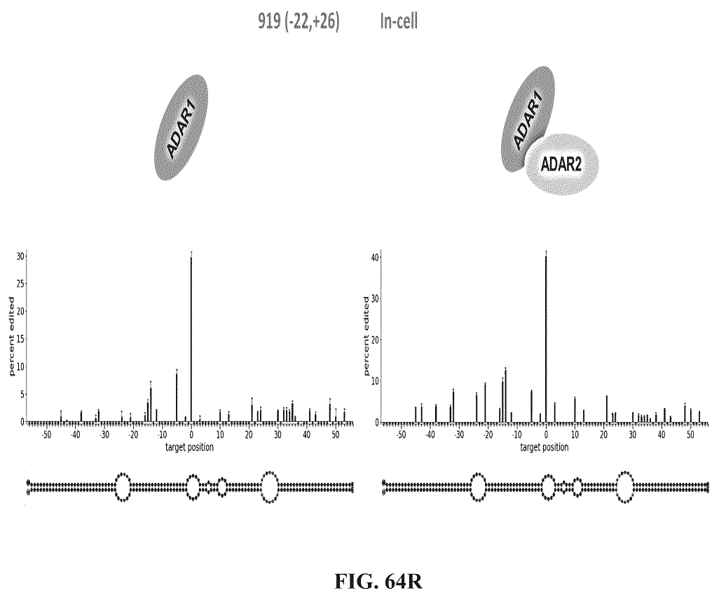

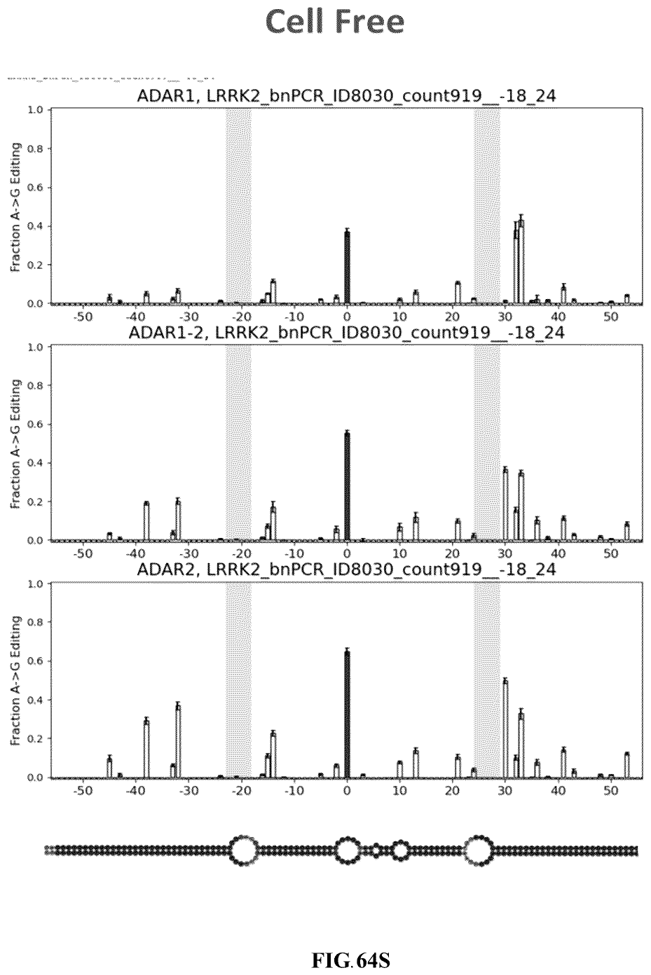

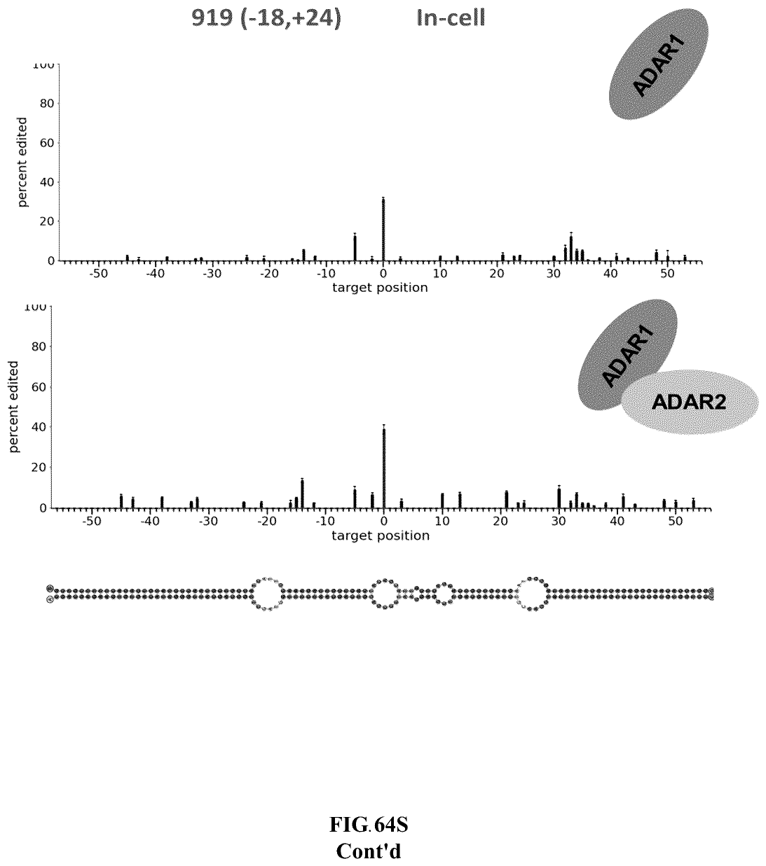

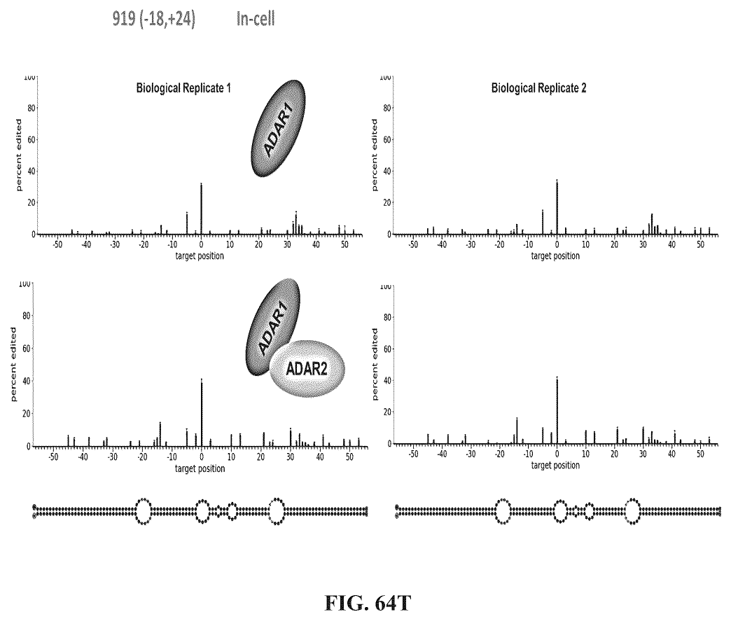

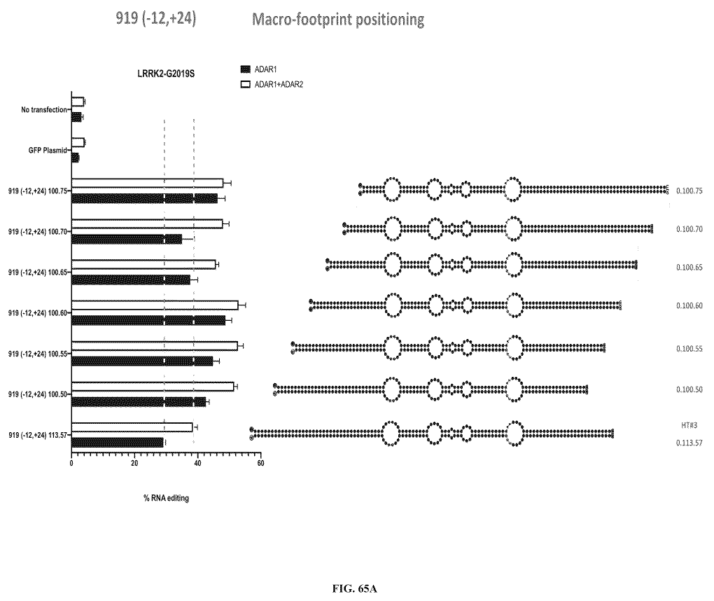

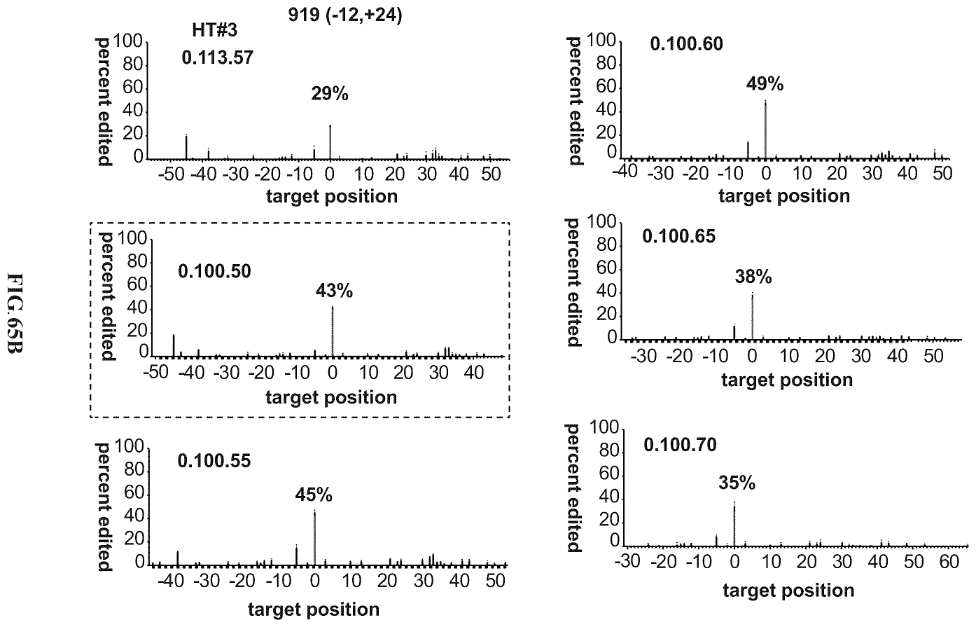

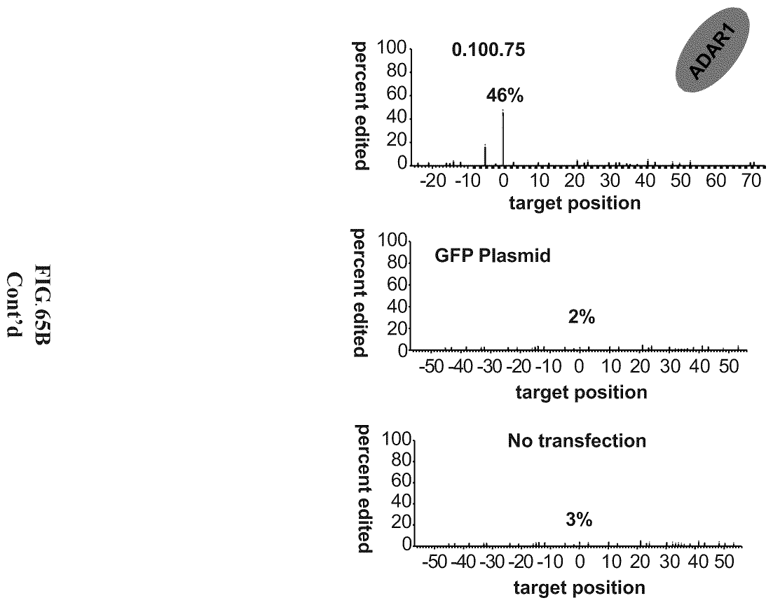

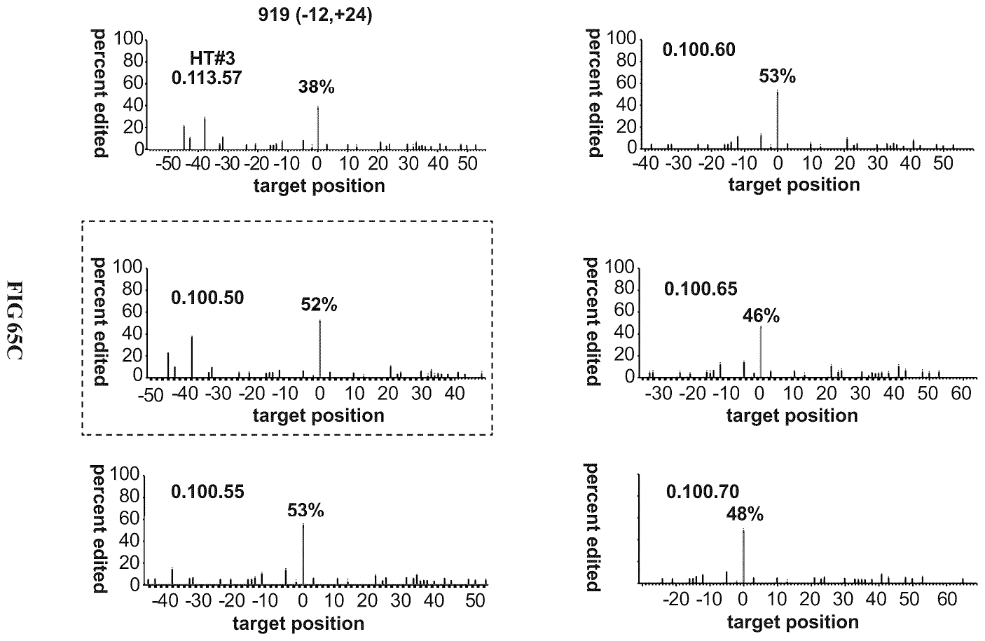

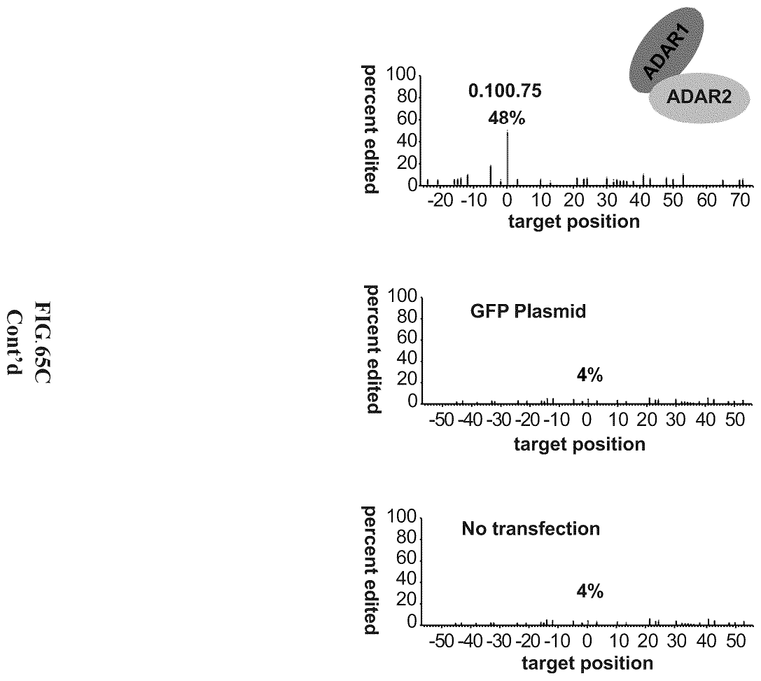

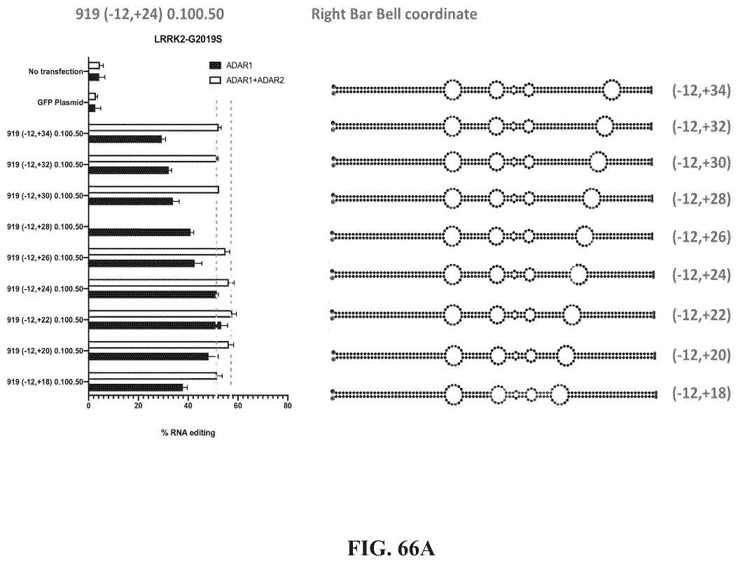

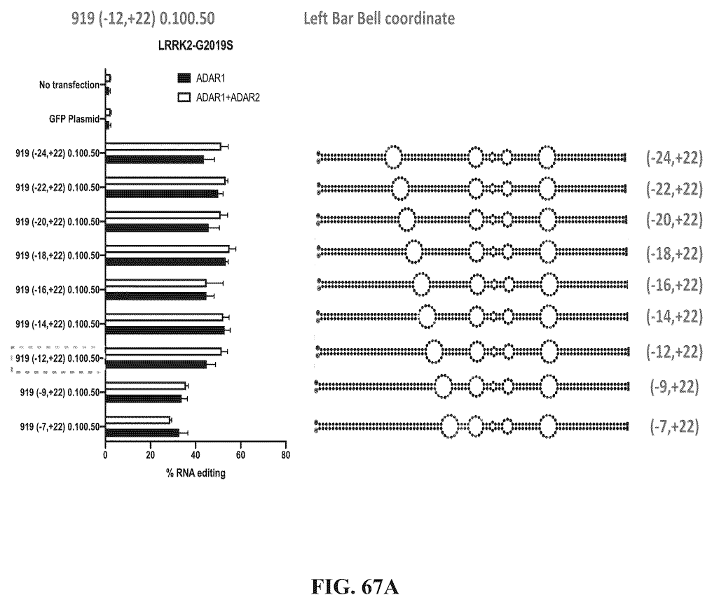

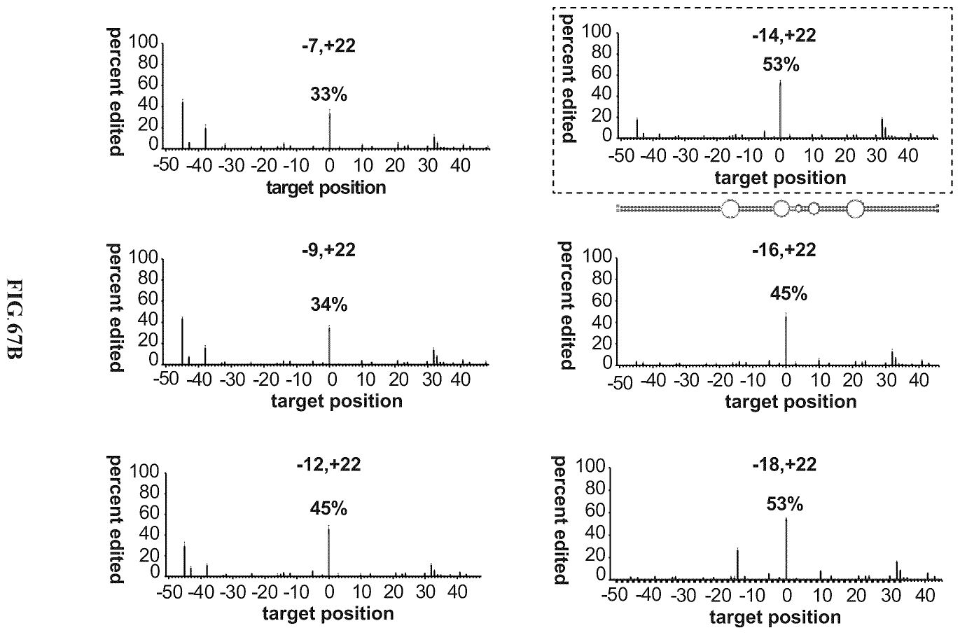

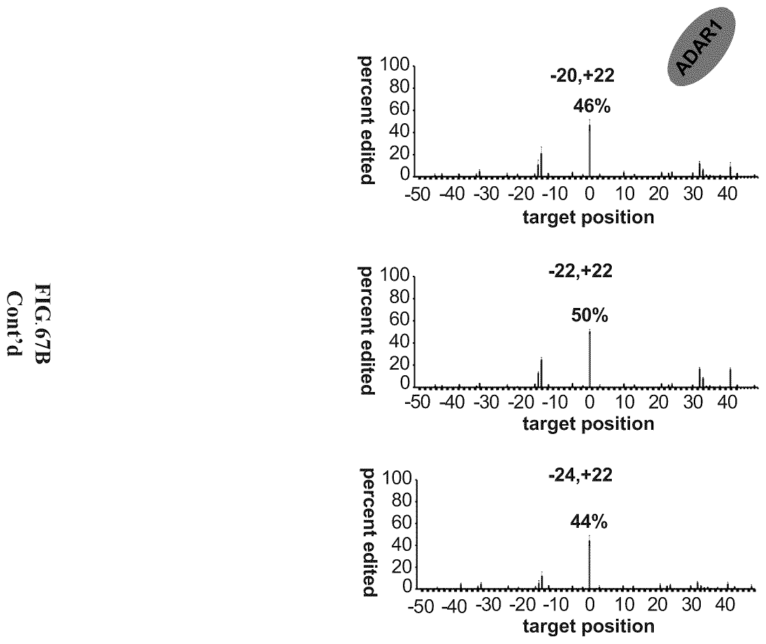

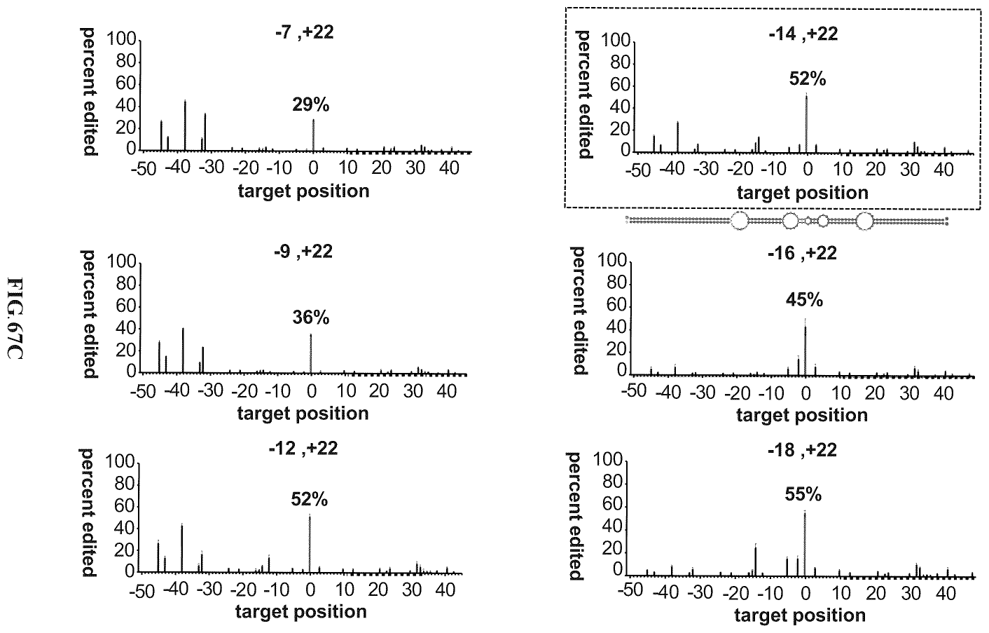

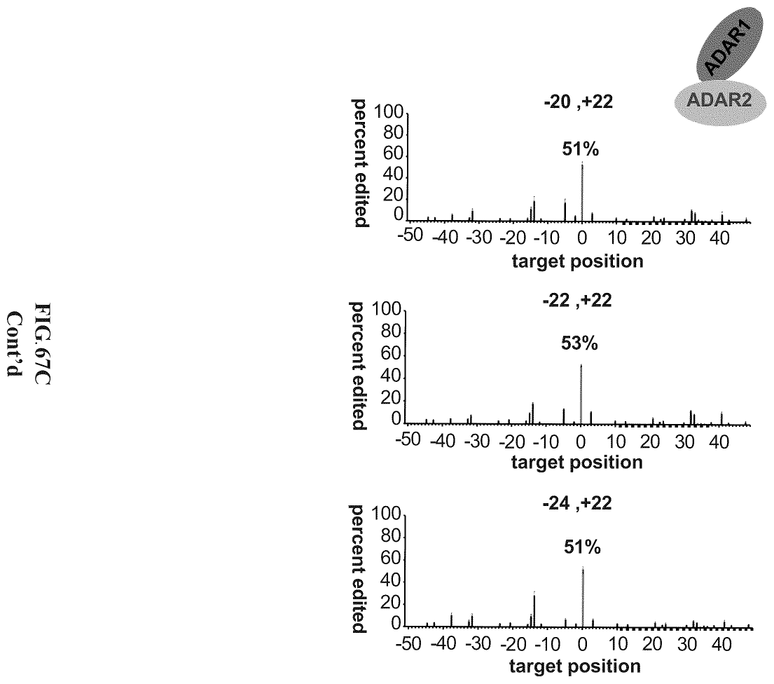

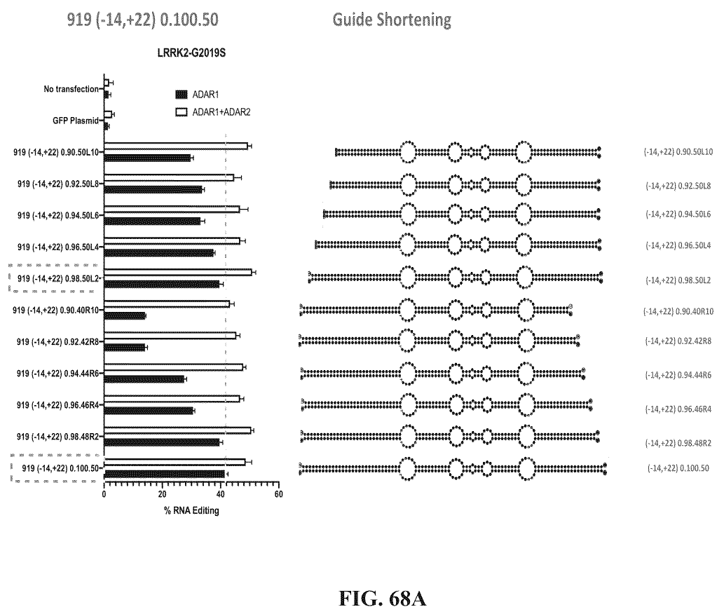

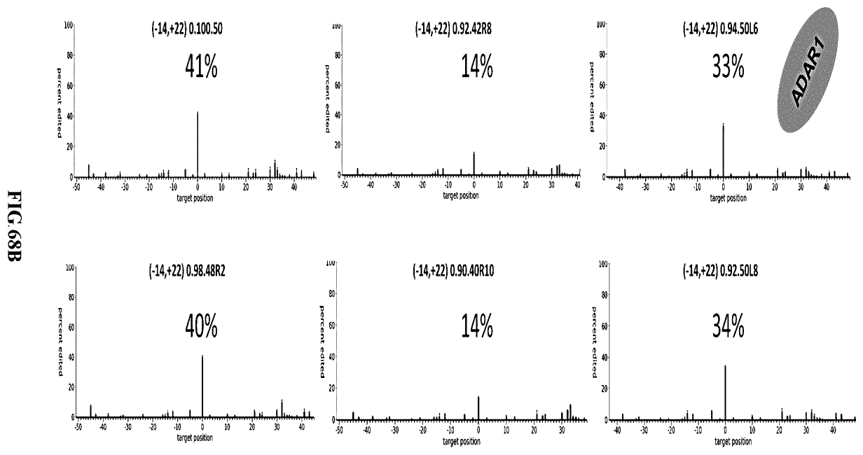

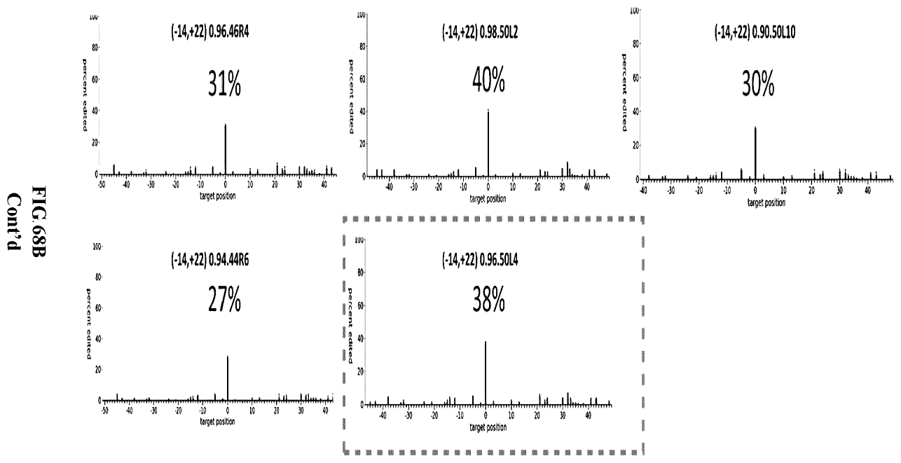

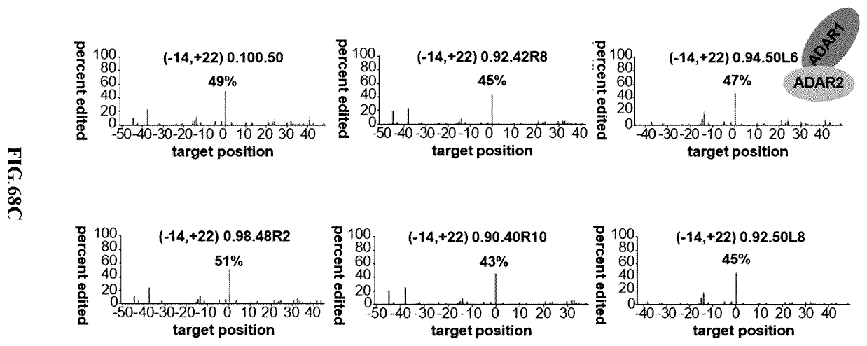

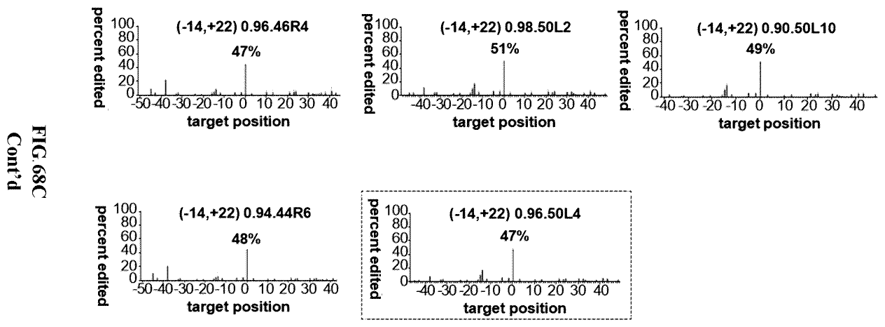

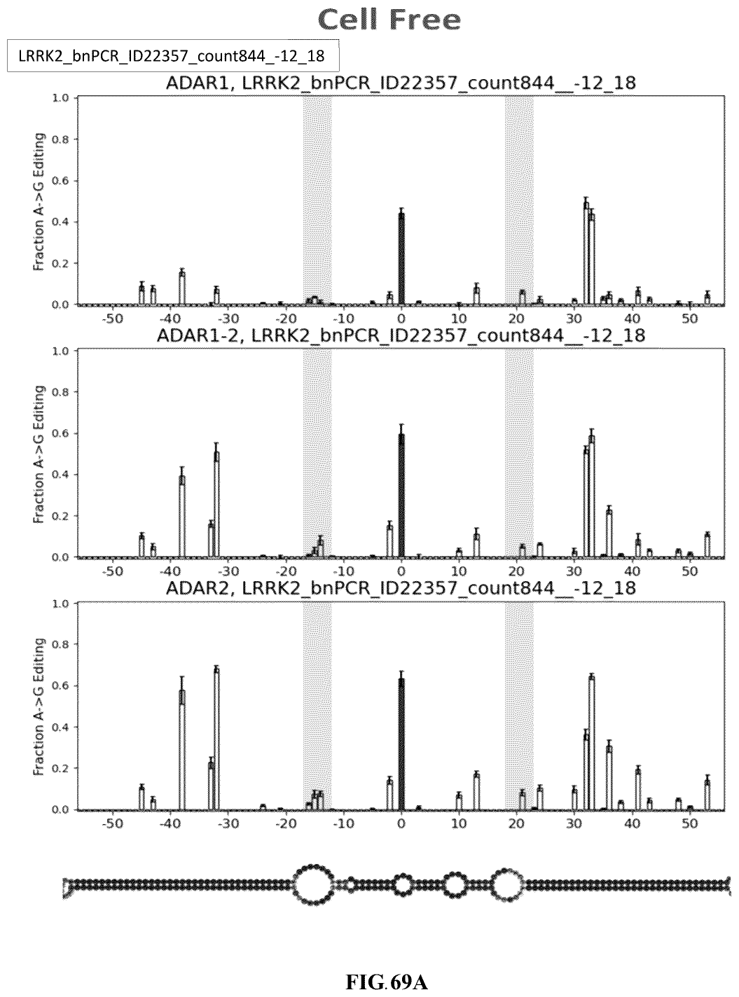

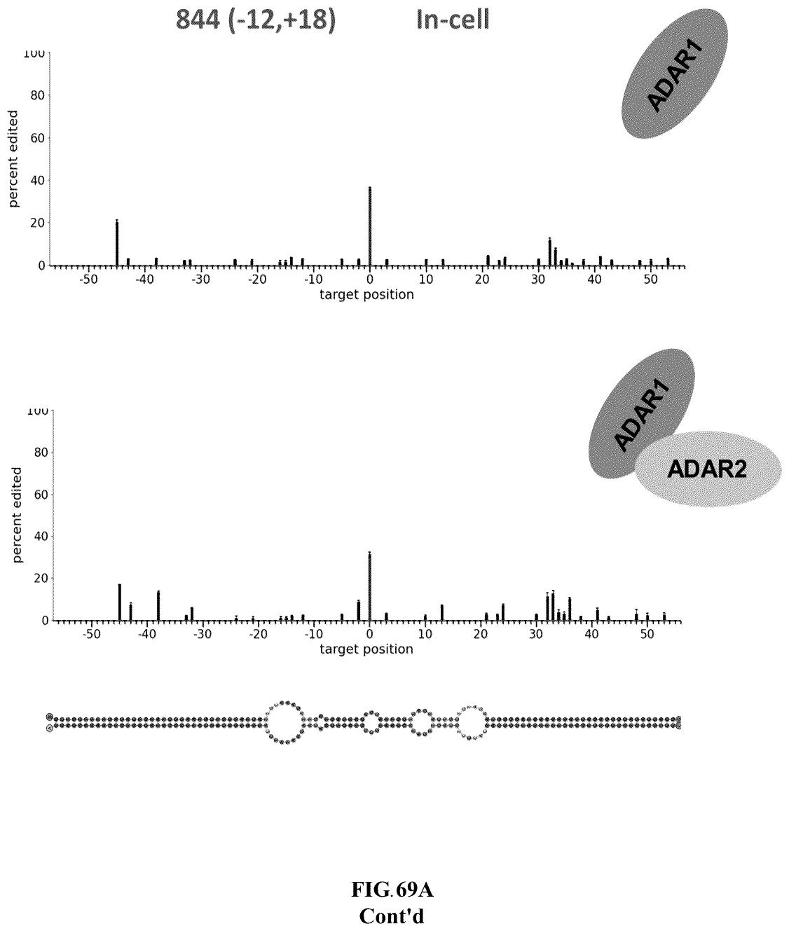

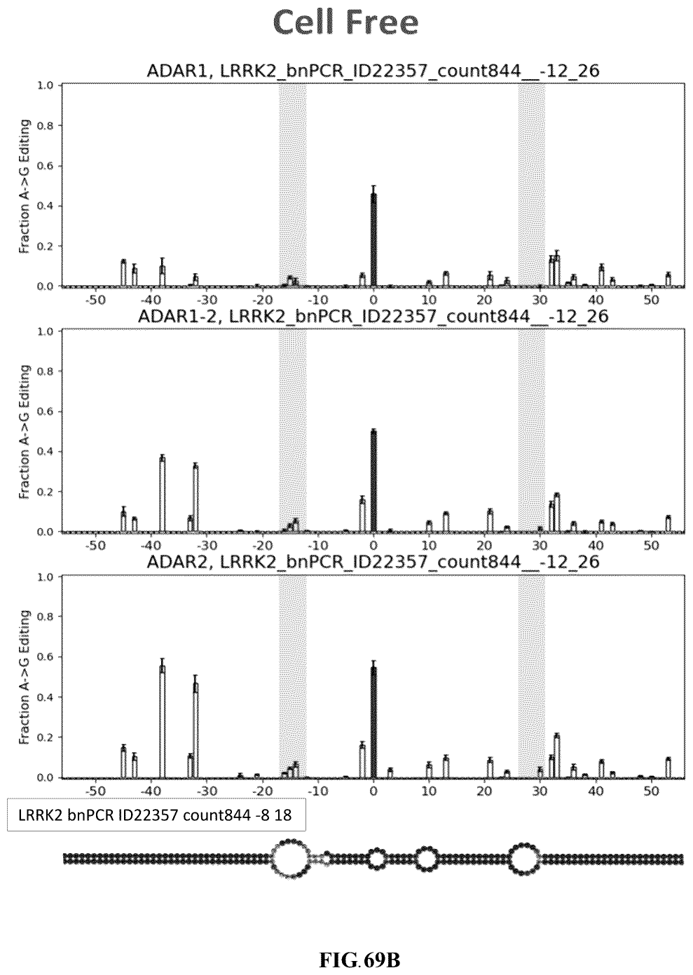

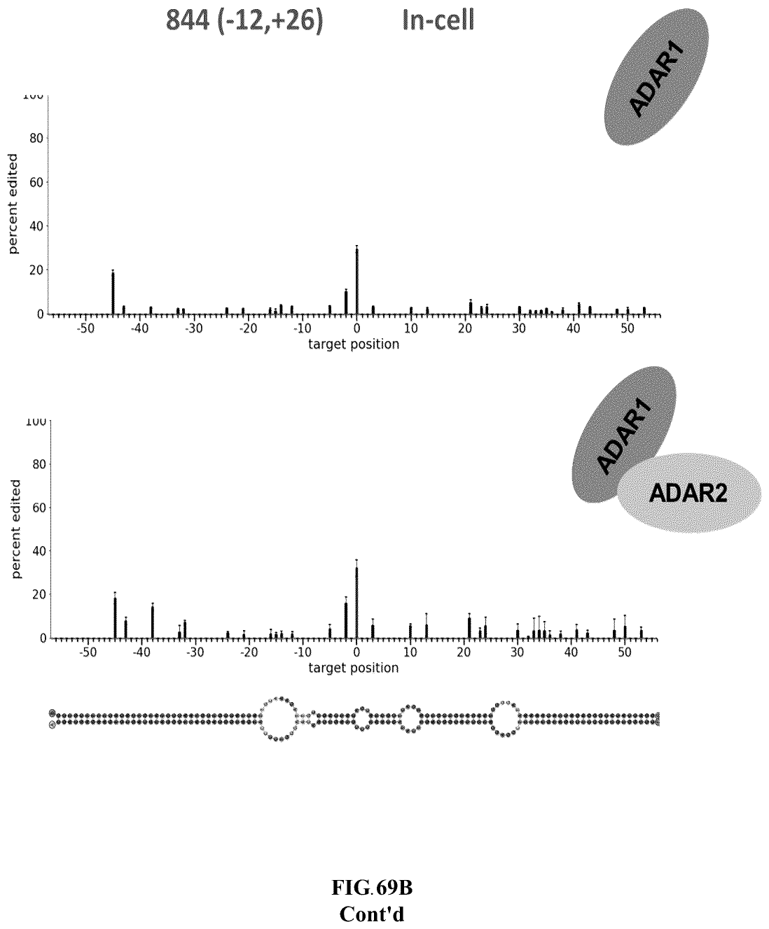

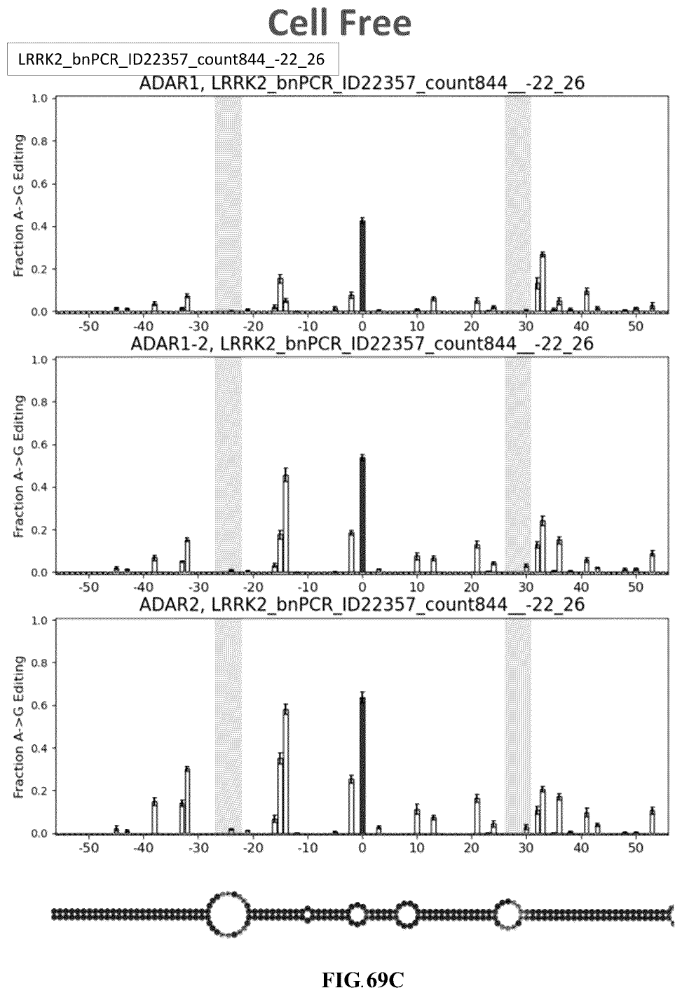

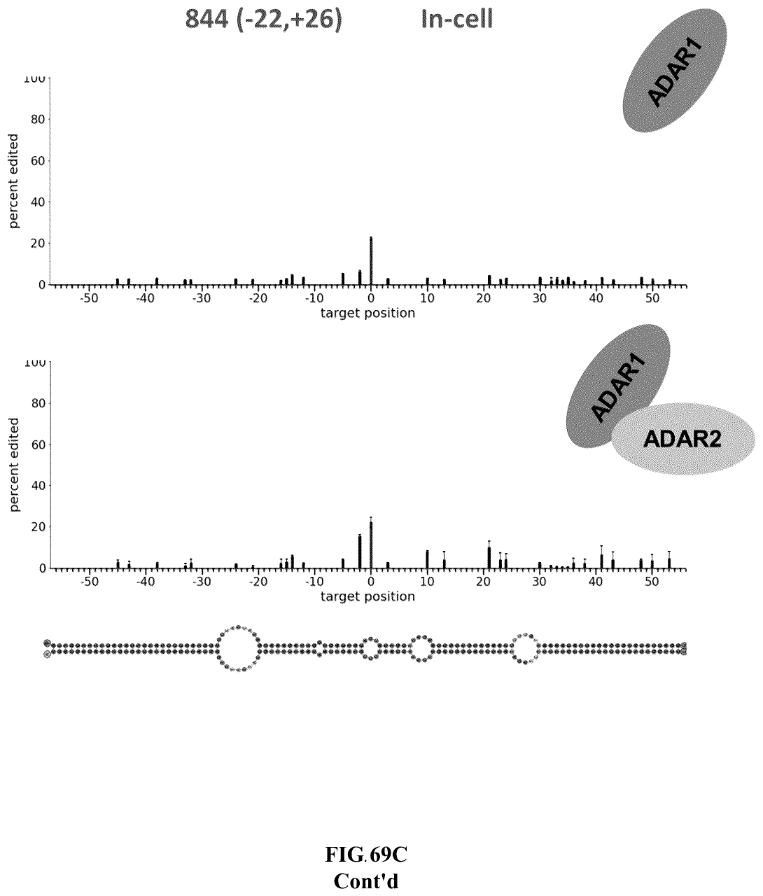

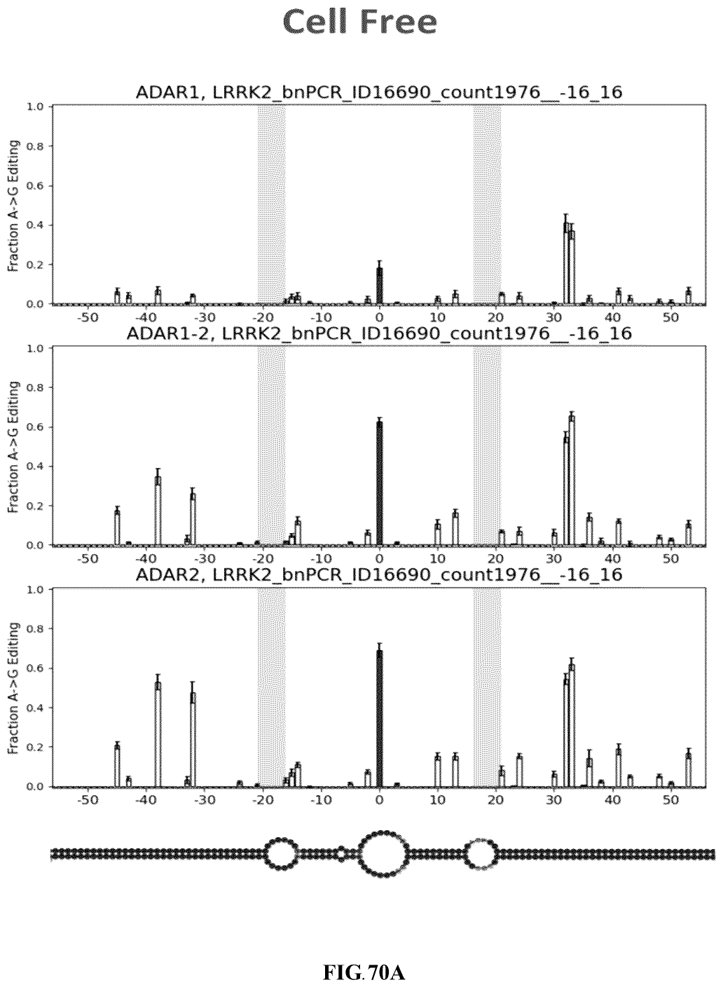

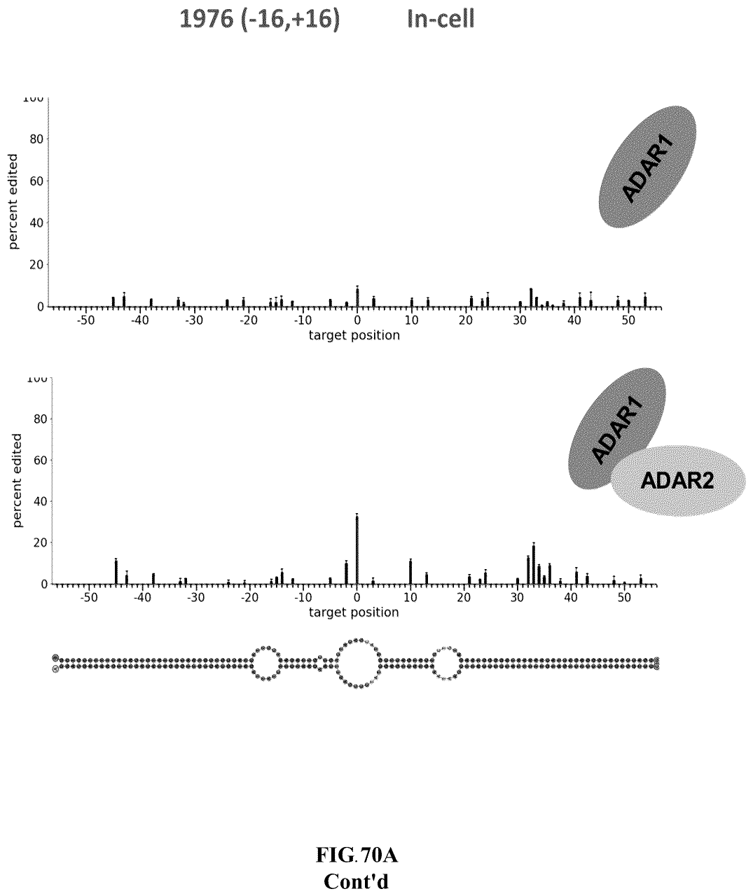

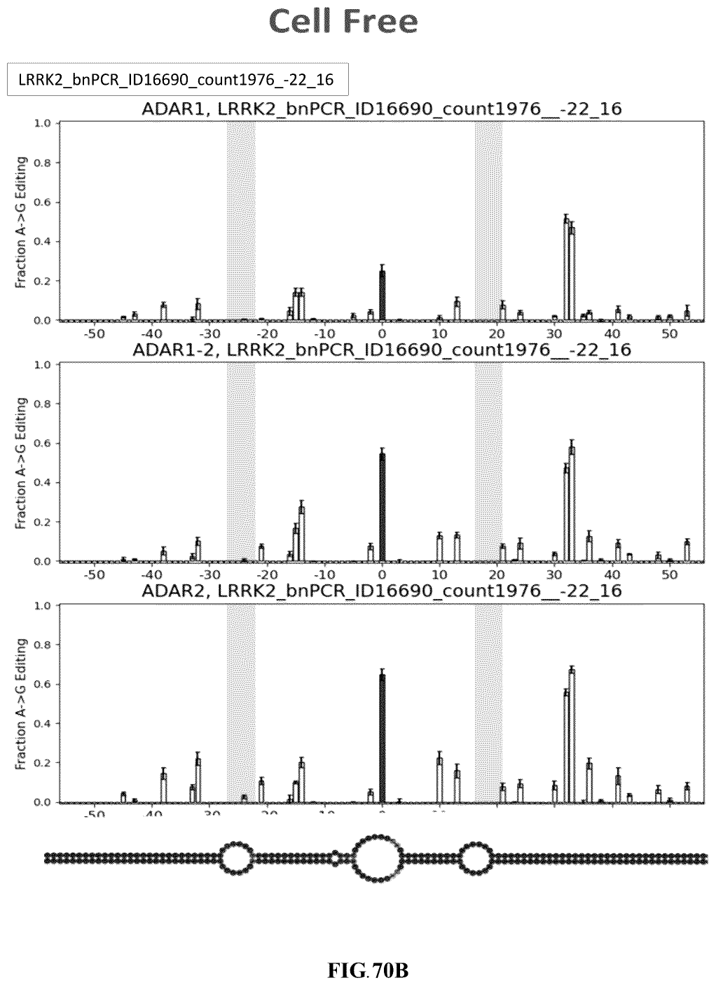

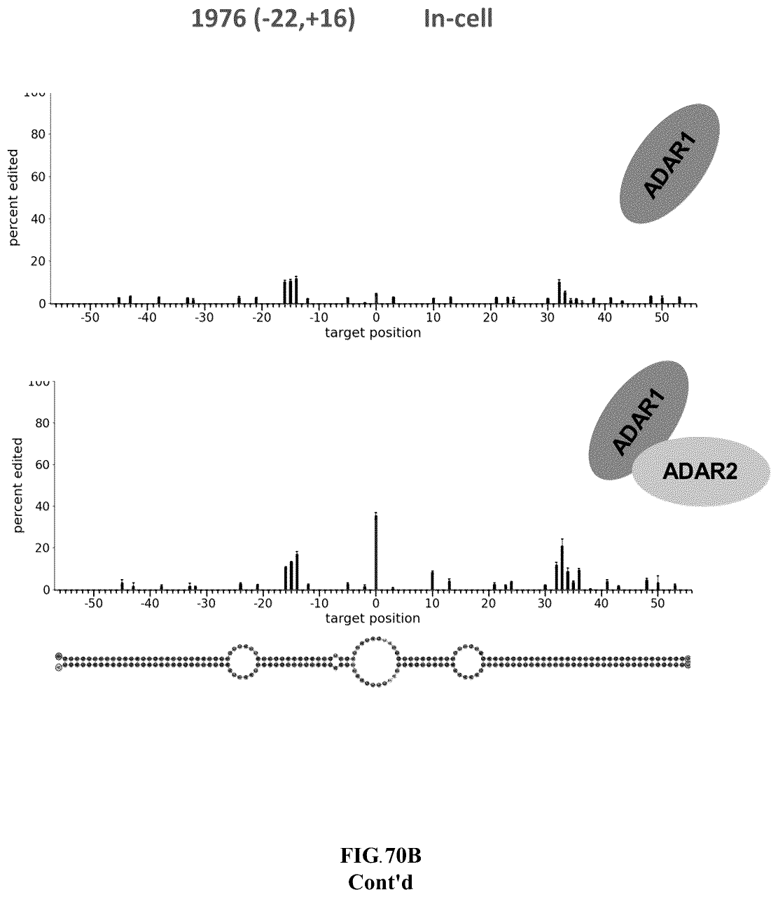

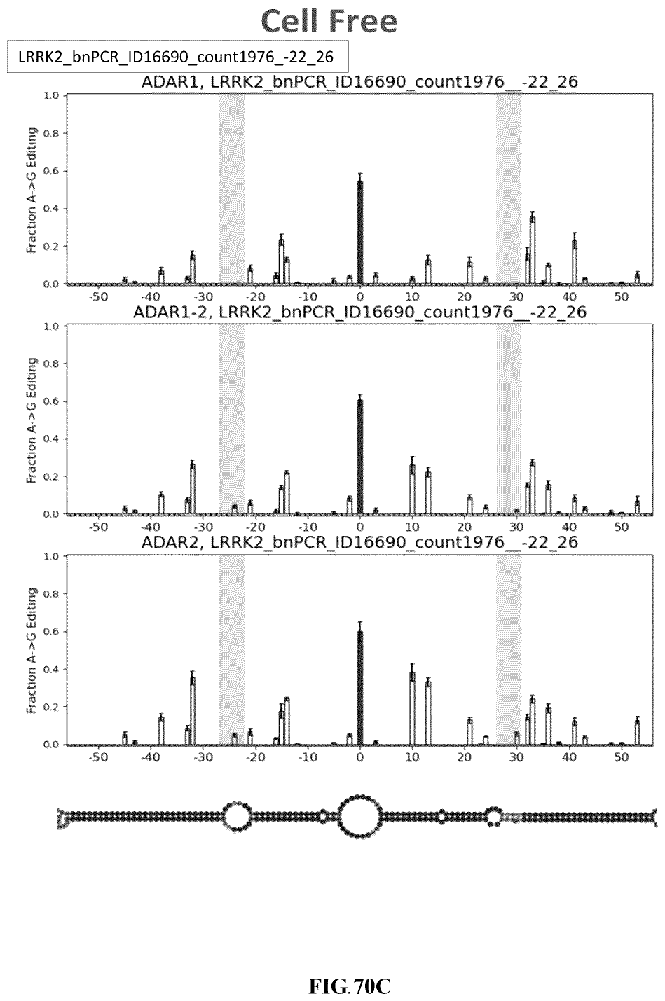

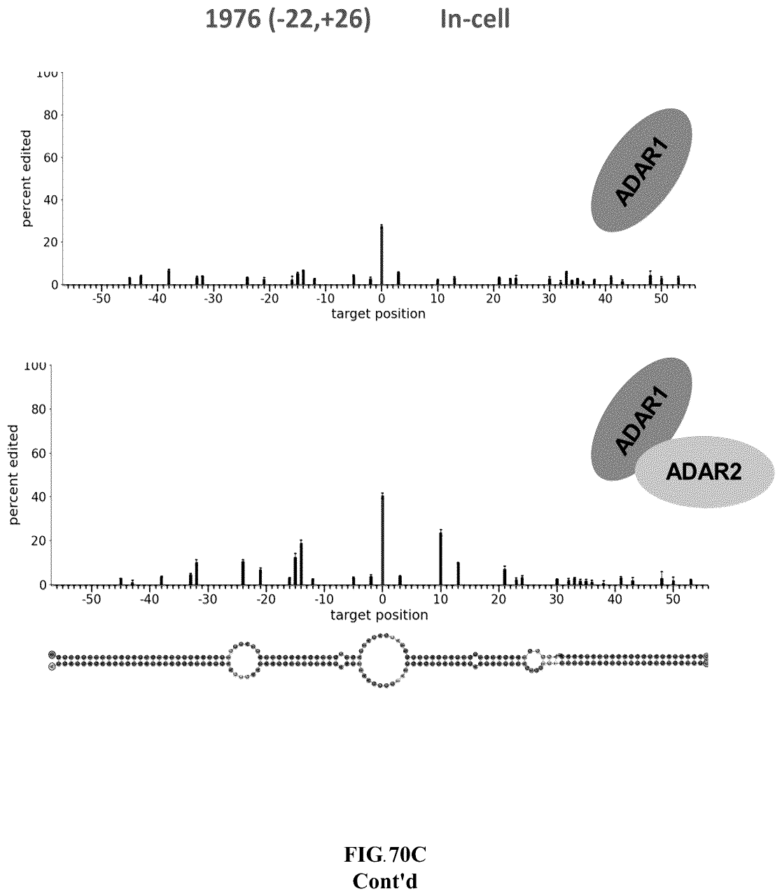

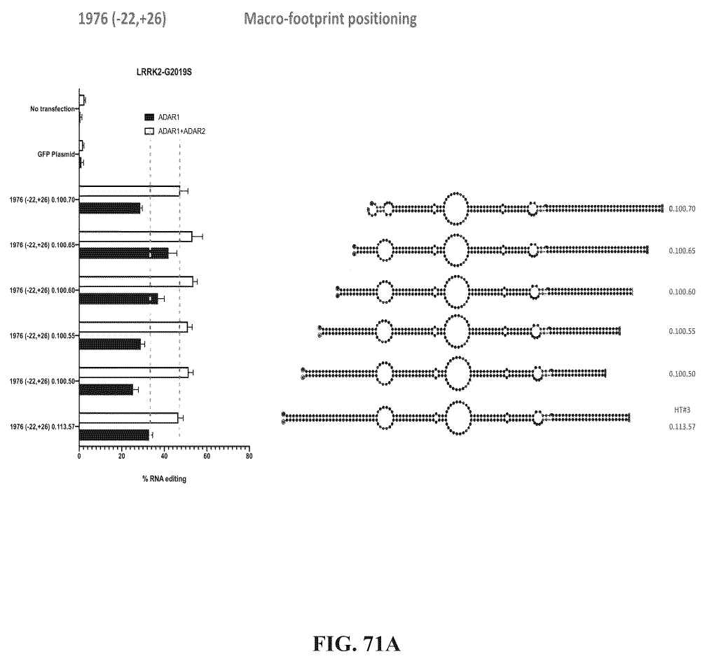

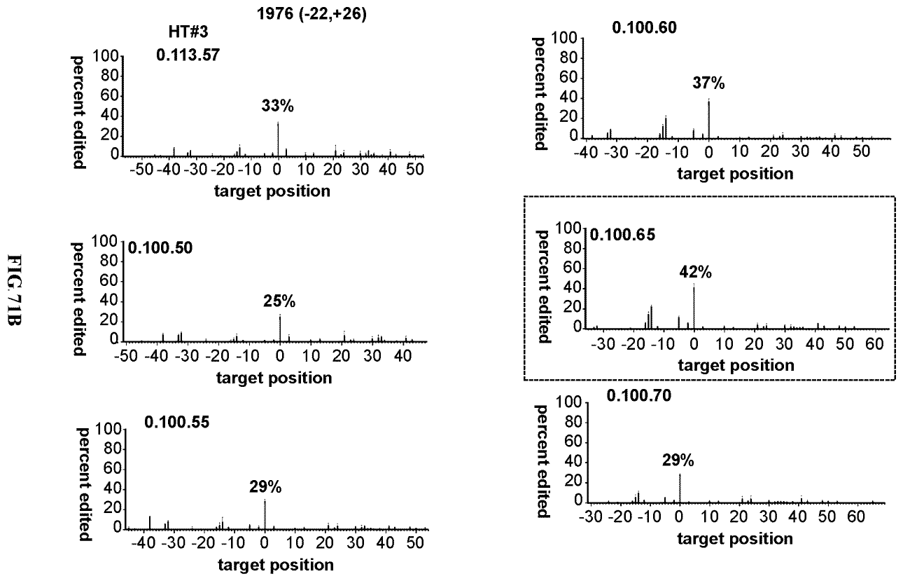

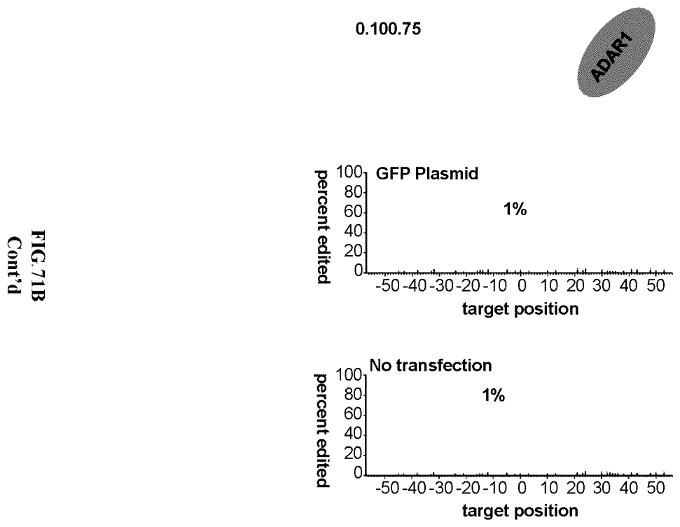

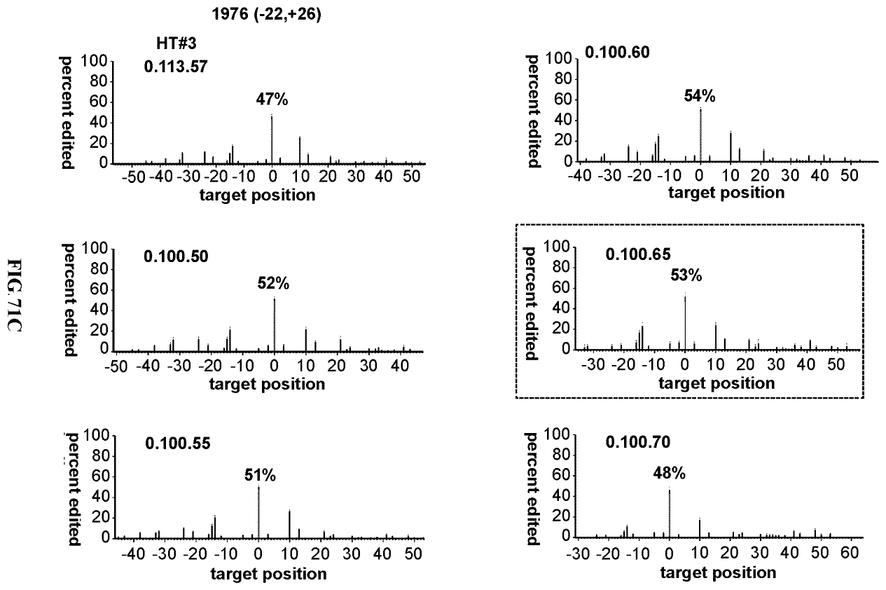

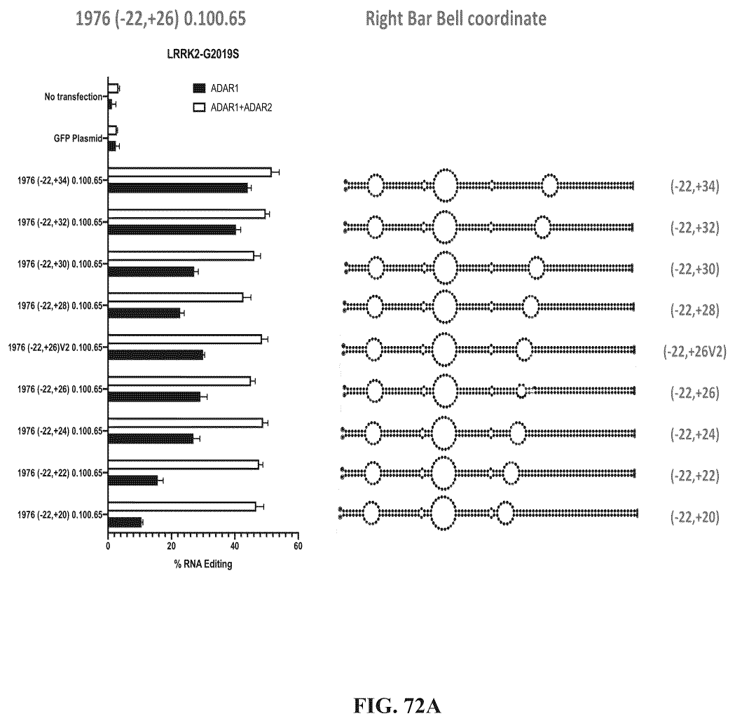

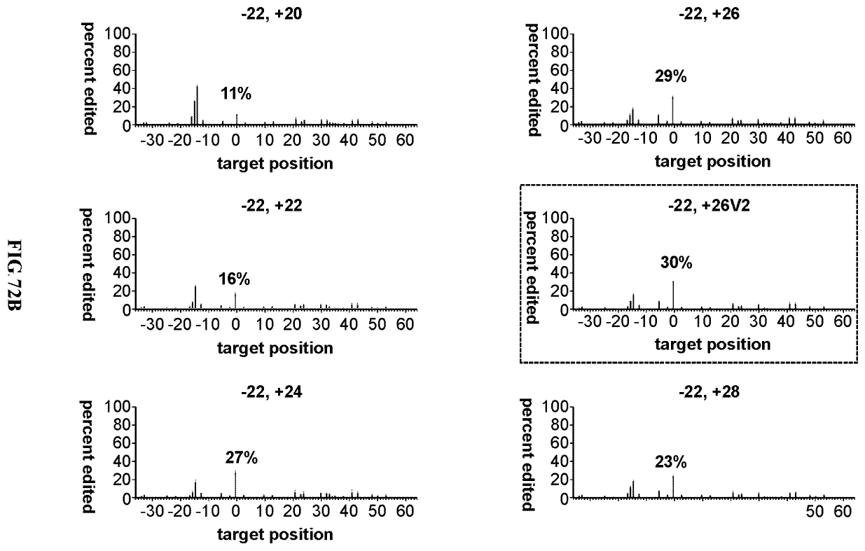

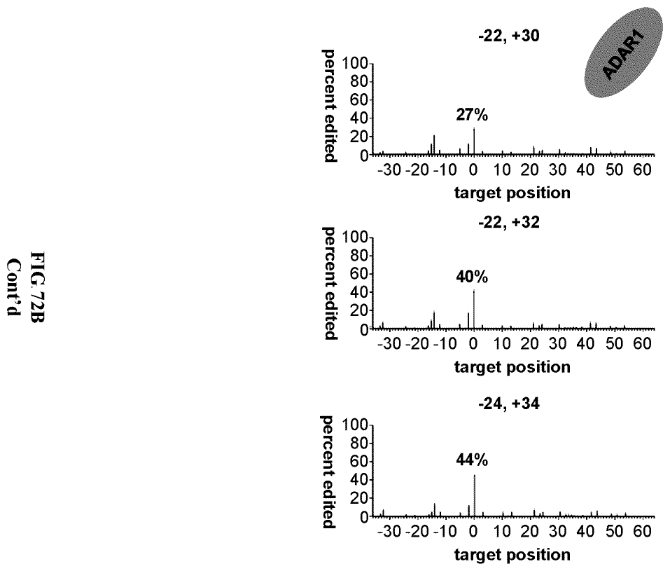

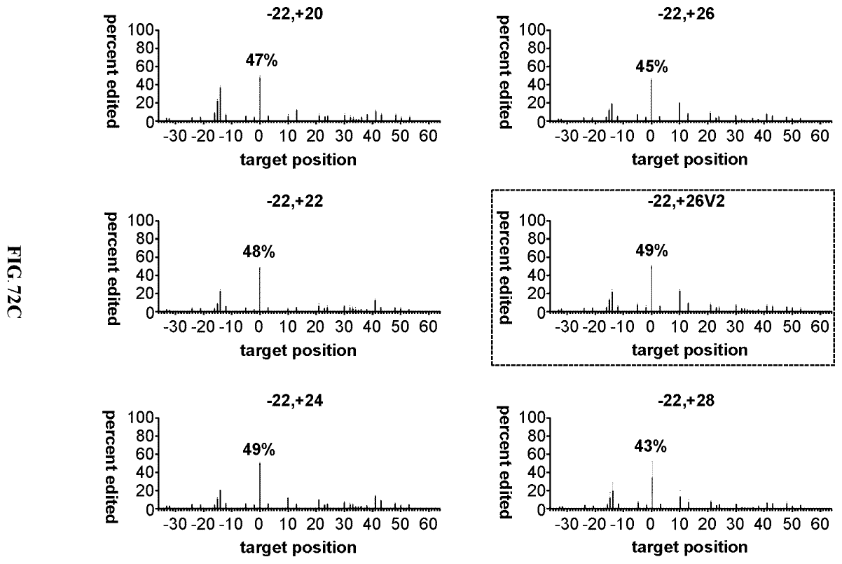

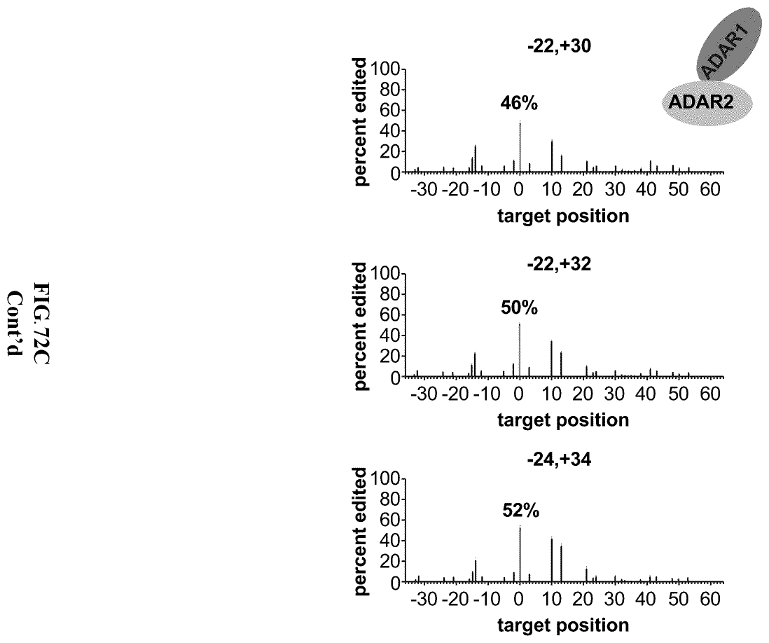

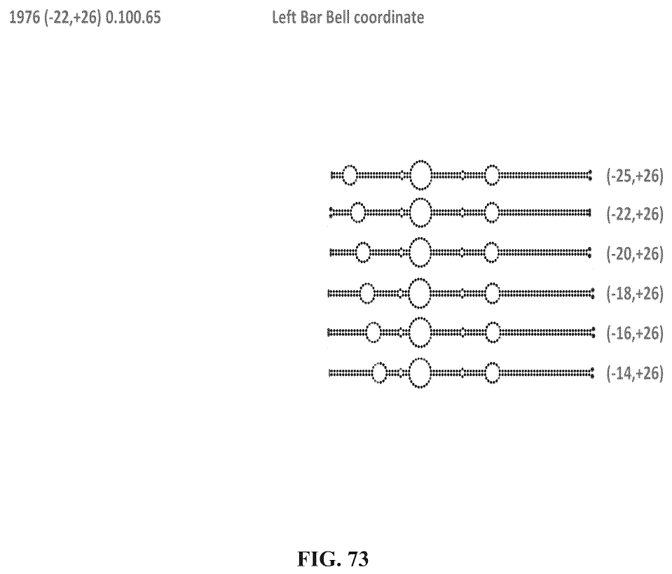

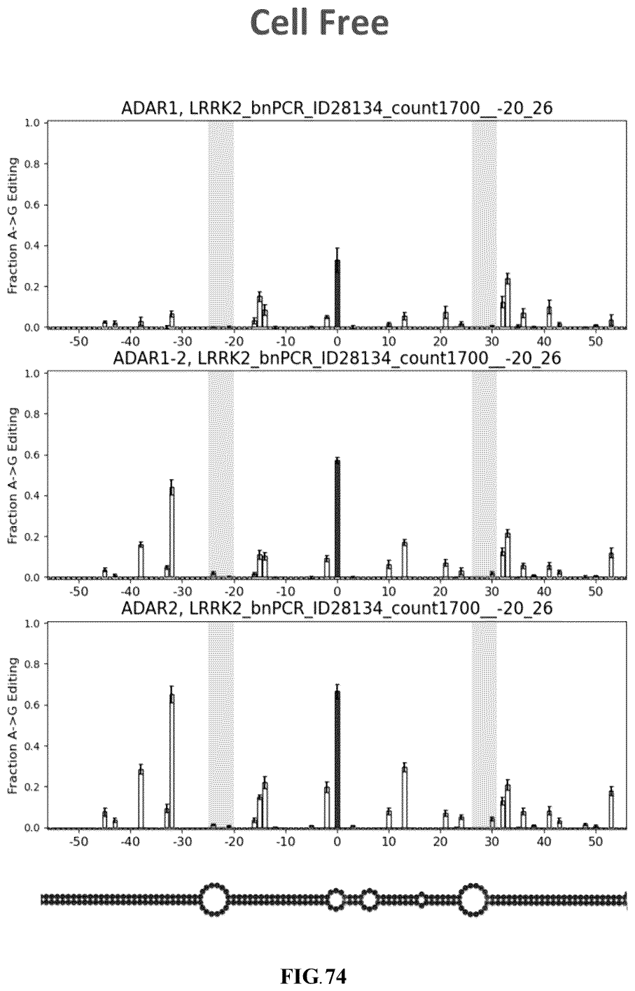

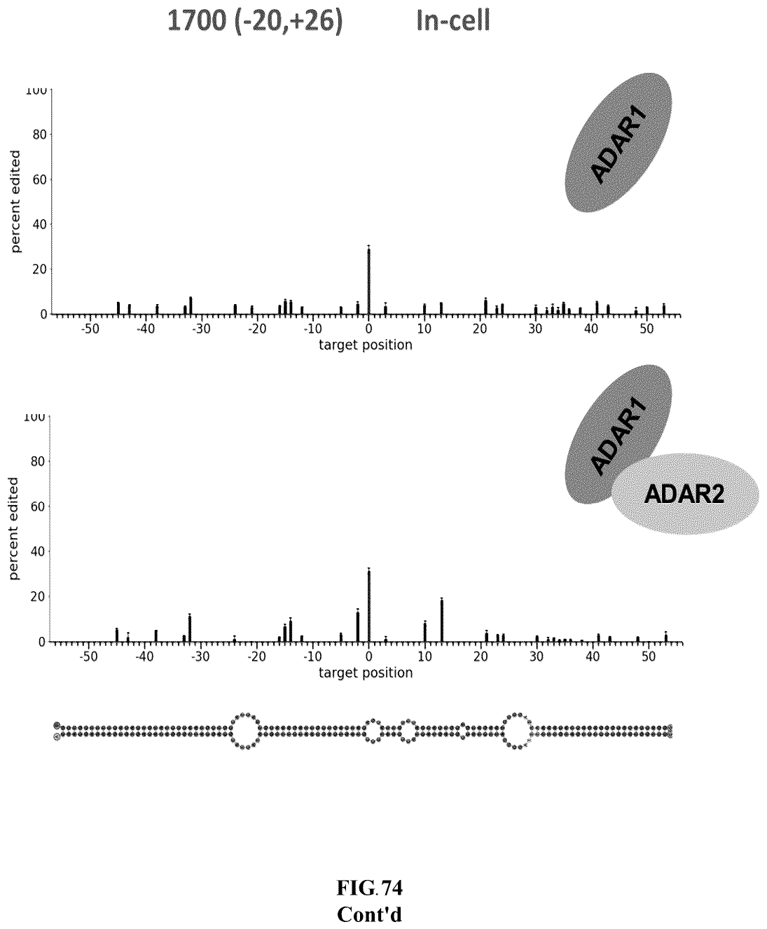

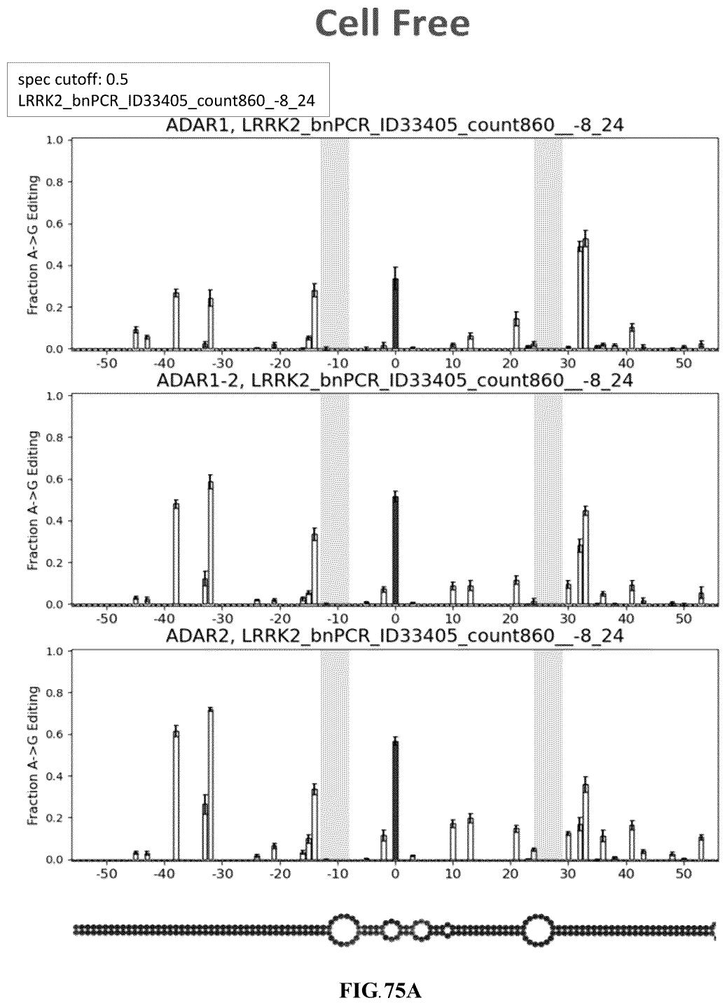

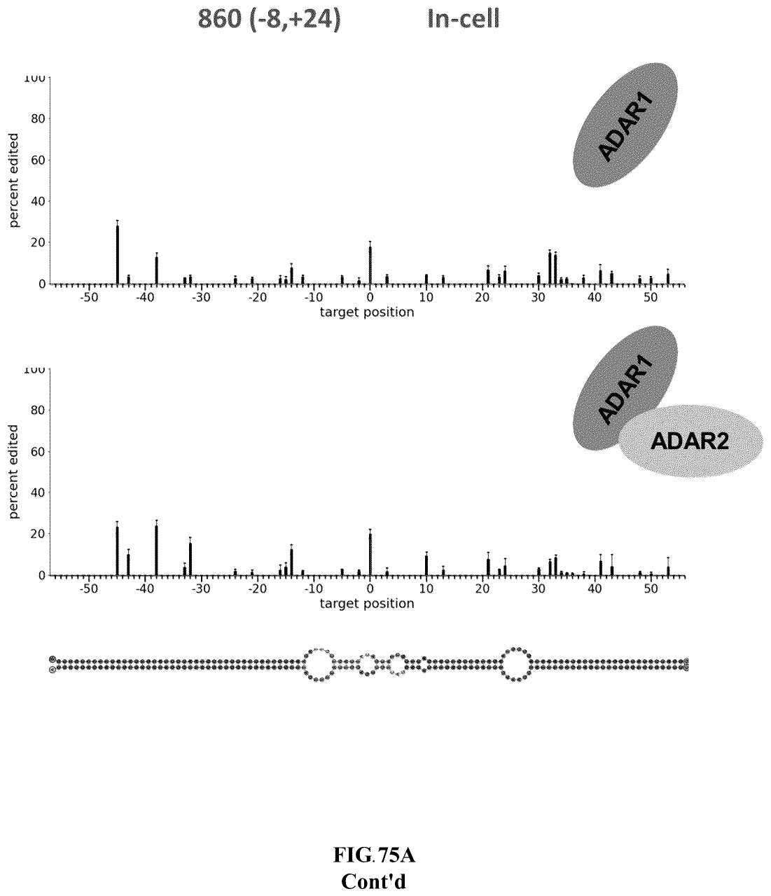

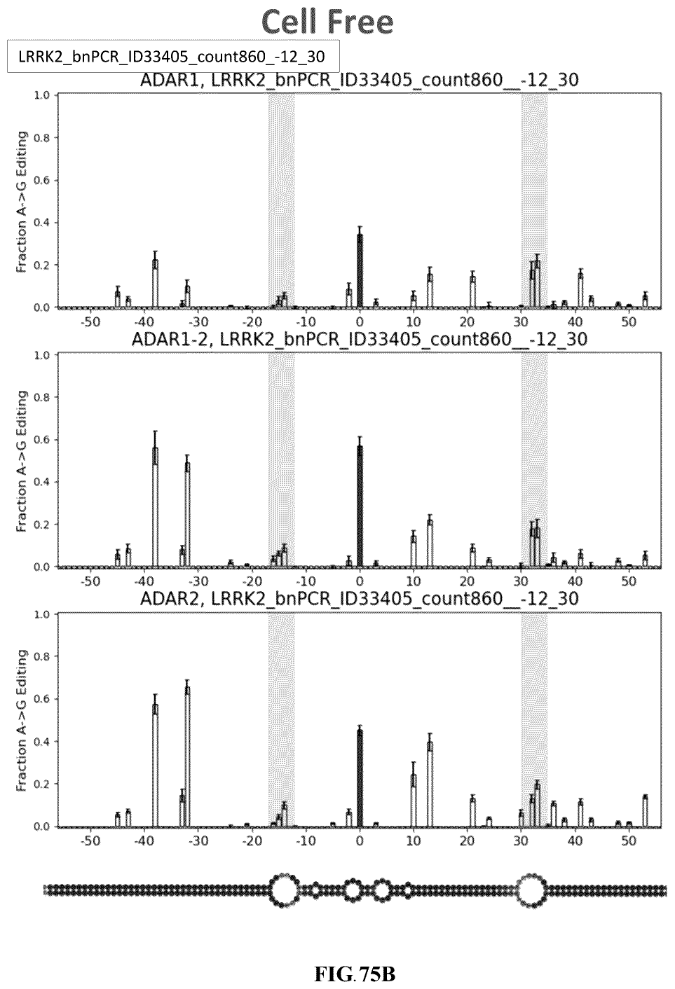

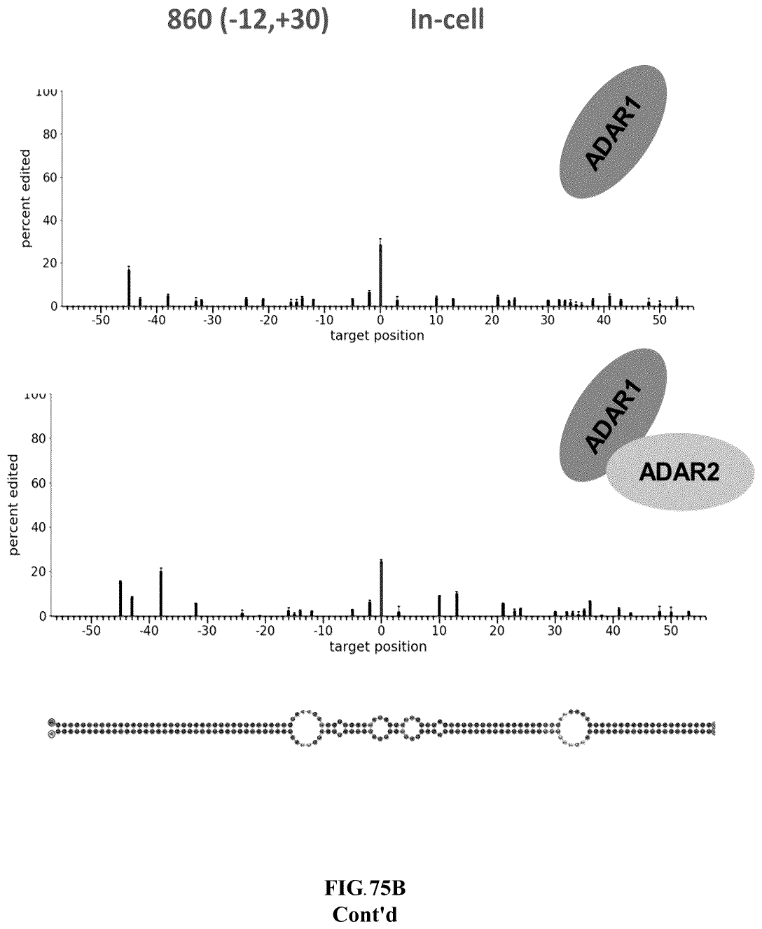

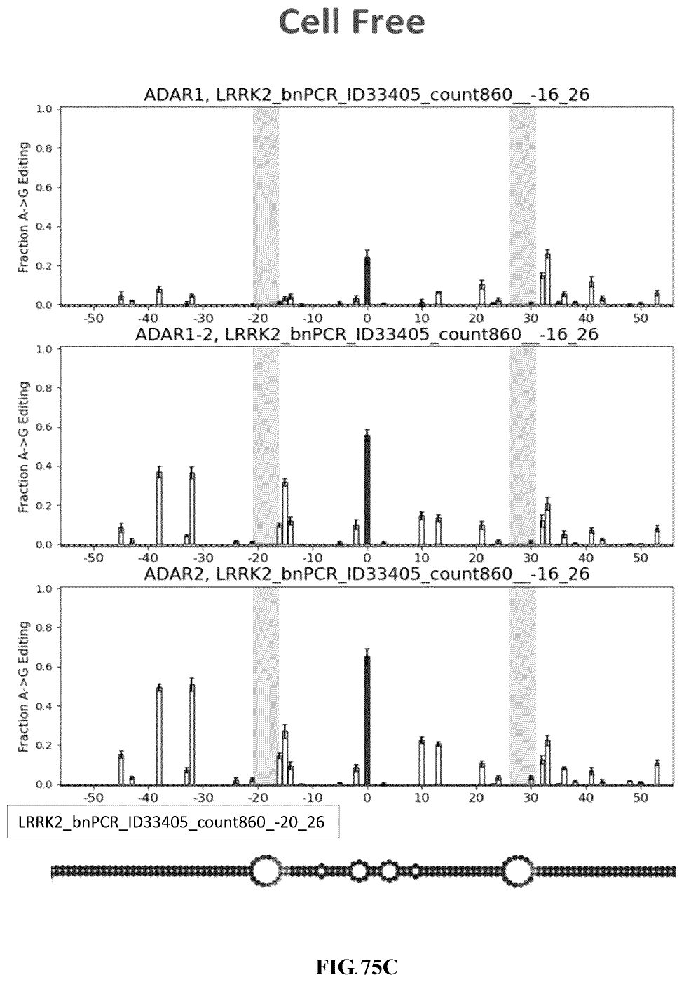

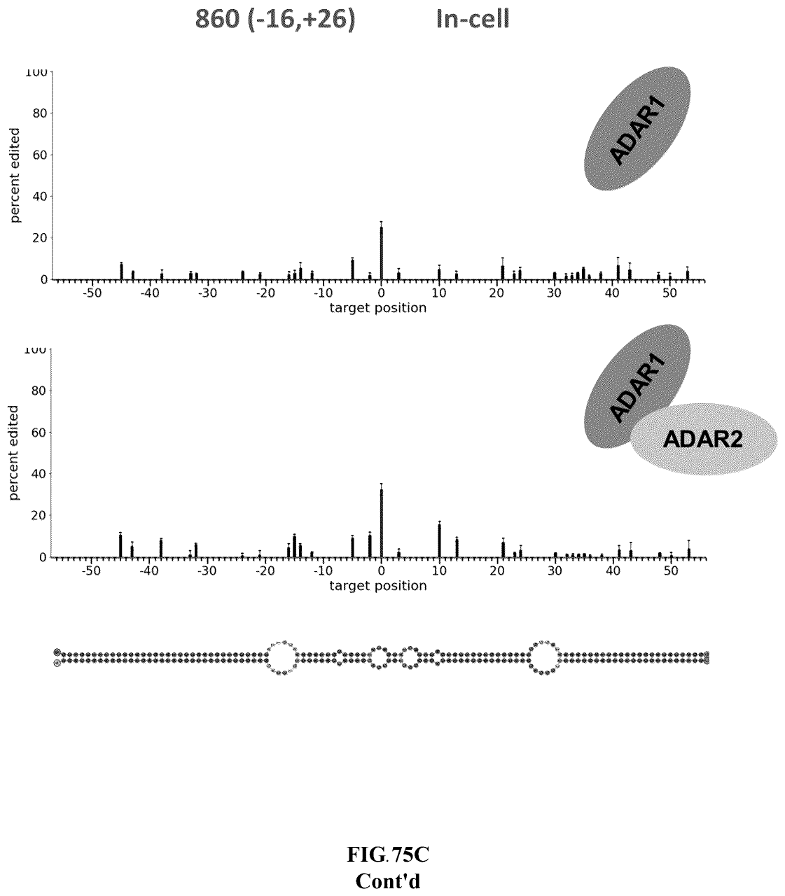

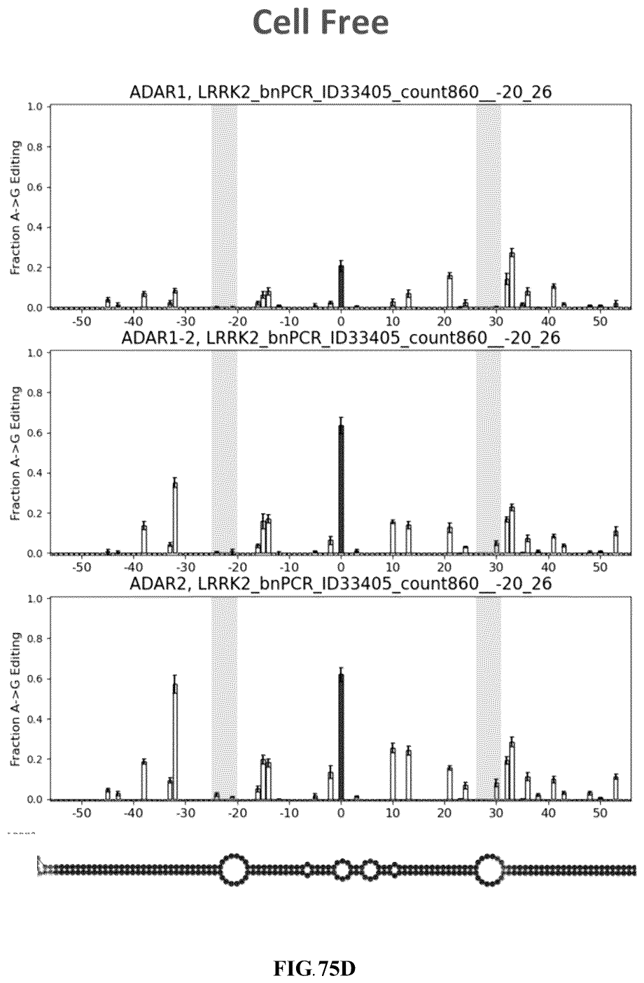

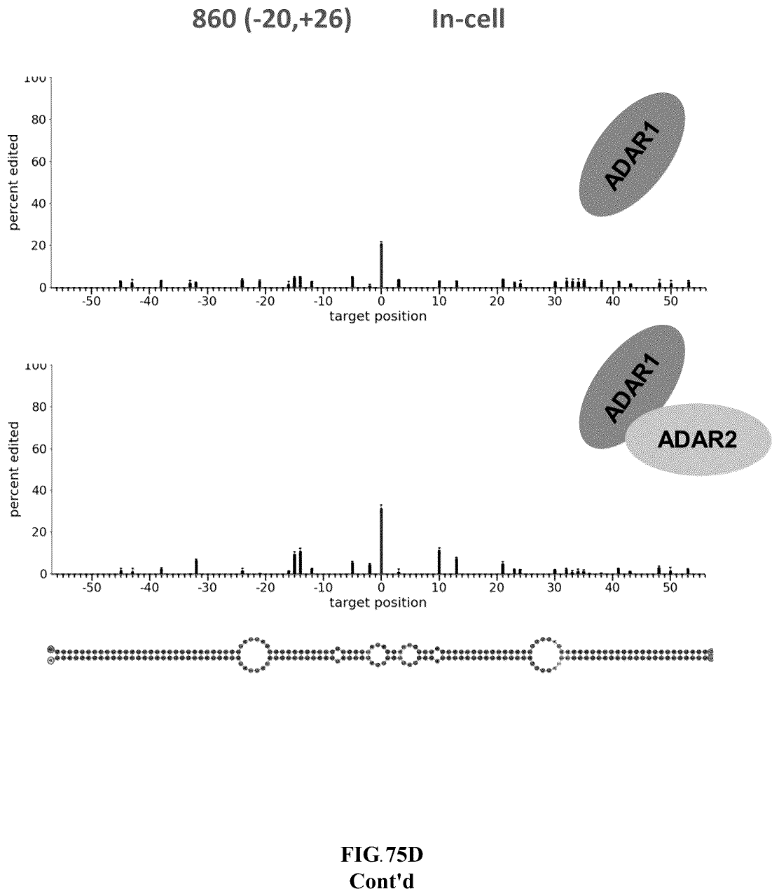

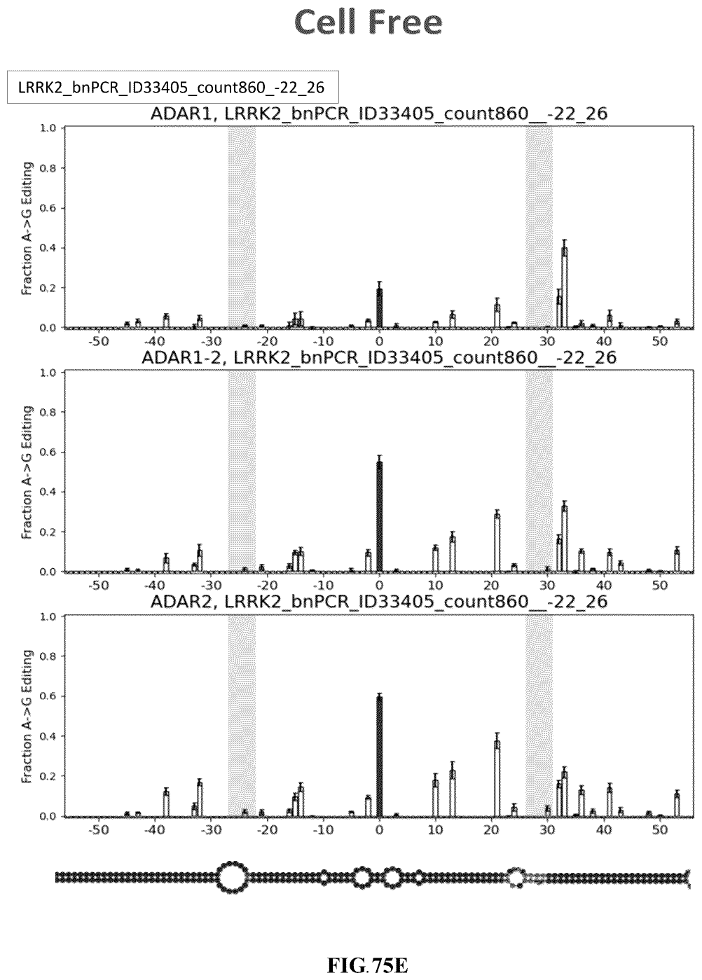

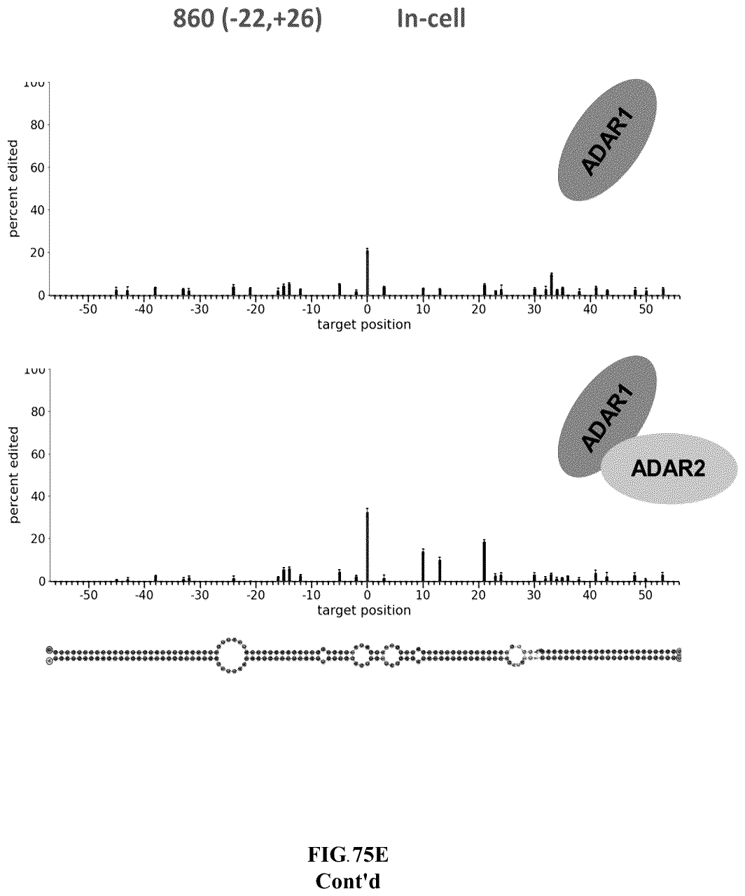

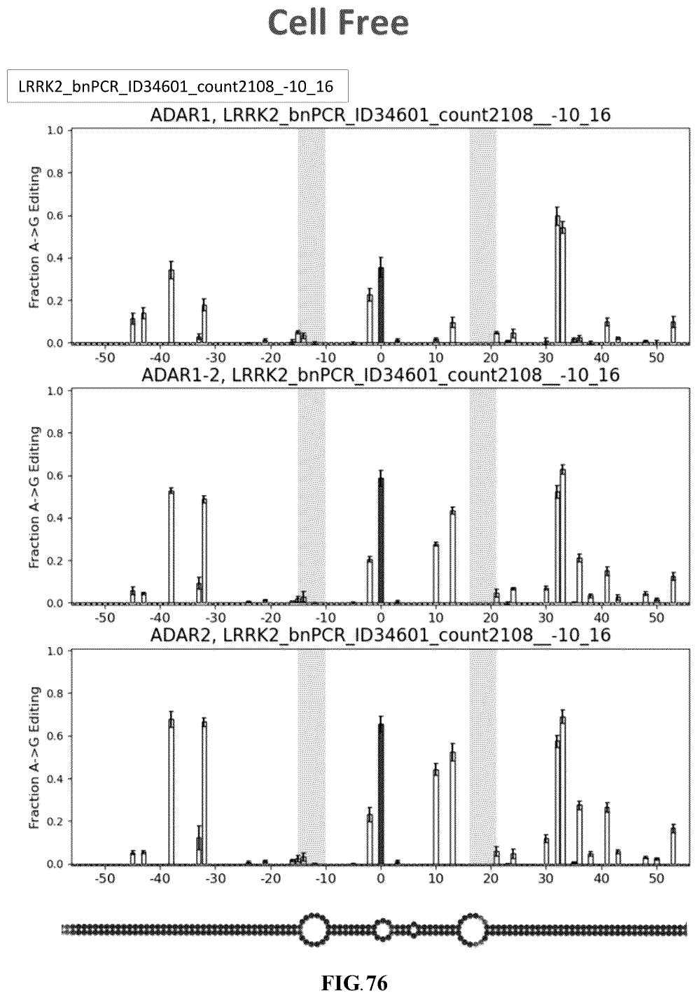

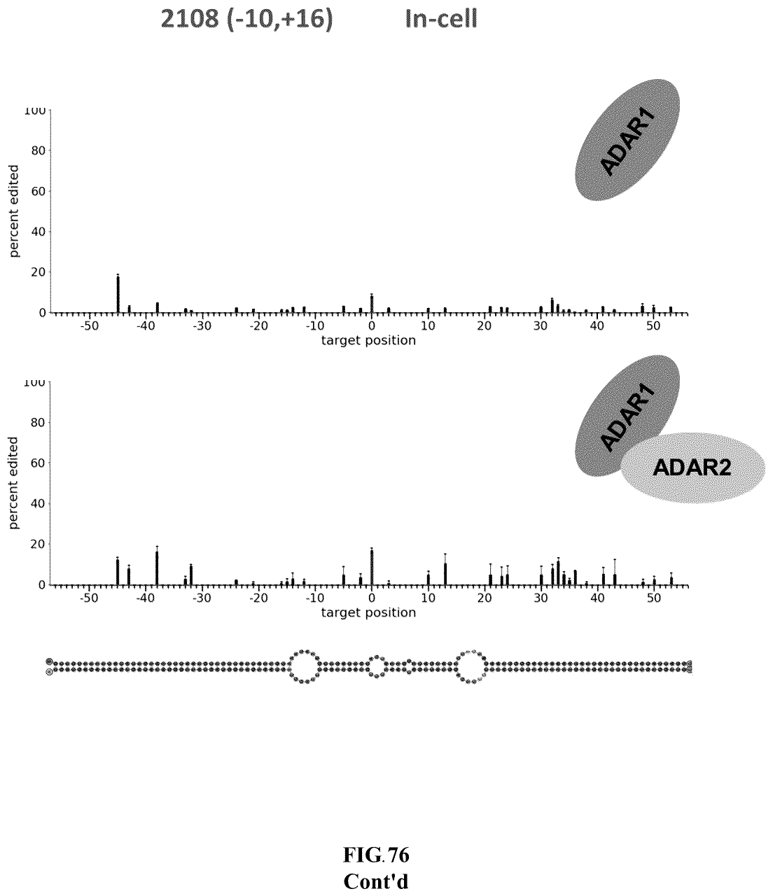

FIG. 47-FIG. 77 depict engineering of LRRK2 variants selected through high throughput screening as described above with respect to guide610. TABLE 7 below recites the sequences of the engineered guide RNAs depicted in FIGS. 47-77. While the engineered guide RNA sequences in TABLE 7 are provided as DNA sequences with a T substituted for each U, the corresponding RNA sequences are also encompassed herein.

TABLE 7
Engineered LRRK2 Guide RNA Sequences

| SEQ | | | | | | |
|---|---|---|---|---|---|---|
| ID | | | | | Left Barbell | Right Barbell |
| NO | FIG. | Guide Name | Sequence | Structural Features (target/guide) | Position | Position |
| 157 | 47A | 2063 (no loops) | CCCTGGTGTGCCCTCTGATG | −49_1-1_wobble_U-G, −8_1- | N/A | N/A |
| | | | TTTTTATCCCCATTCTACAG | 1_mismatch_C-U, −3_1-1_wobble_U-G, | | |
| | | | CAGTAGTGTGCAGTGCCGTG | 0_1-1_mismatch_A-C, 4_1- | | |
| | | | GTCATCAATCTTTGCAATGA | 1_wobble_U-G, 8_1-1_mismatch_U-U, | | |
| | | | TGGCAGCATTGGGATACAGT | 11_1-1_mismatch_G-G | | |
| | | | GTGAAGAGCAGCA | | | |
| 158 | 47B | 2063 (−14, +26) | CCCTGGTGTGCCCTCTGATG | −49_1-1_wobble_U-G, −14_6- | −14 | 26 |
| | | | TTTTTTAGGGGATTCTACAG | 6_internal_loop-symmetric_UGCAAA- | | |
| | | | CAGTAGTGTGCAGTGCCGTG | AAACGU, −8_1-1_mismatch_C-U, | | |
| | | | GTCATCAATCAAACGTATGA | −3_1-1_wobble_U-G, 0_1- | | |
| | | | TGGCAGCATTGGGATACAGT | 1_mismatch_A-C, 4_1-1_wobble_U-G, | | |
| | | | GTGAAGAGCAGCA | 8_1-1_mismatch_U-U, 11_1- | | |
| | | | | 1_mismatch_G-G, 26_6- | | |
| | | | | 6_internal_loop-symmetric_GGGGAU- | | |
| | | | | UAGGGG | | |
| 238 | 48A | 1590 (no loops) | CCCTGGTGTGCCCTCTGATG | −49_1-1_wobble_U-G, −3_1- | N/A | 6 |
| | | | TTTTTATCCCCATTCTACAA | 1_wobble_U-G, 0_1-1_mismatch_A-C, | | |
| | | | CAGTACGGTGAAGTGCCGT | 4_1-1_wobble_U-G, 6_5- | | |
| | | | GGTCAGCAATCTTTGCAATG | 5_internal_loop-symmetric_GCUCA- | | |
| | | | ATGGCAGCATTGGGATACA | GGUGA, 17_1-1_mismatch_C-A | | |
| | | | GTGTGAAGAGCAGCA | | | |
| 239 | 48B | 1590 (−14, +26) | CCCTGGTGTGCCCTCTGATG | −49_1-1_wobble_U-G, −14_6- | −14 | 26 |
| | | | TTTTTTAGGGGATTCTACAA | 6_internal_loop-symmetric_UGCAAA- | | |
| | | | CAGTACGGTGAAGTGCCGT | AAACGU, −3_1-1_wobble_U-G, 0_1- | | |
| | | | GGTCAGCAATCAAACGTAT | 1_mismatch_A-C, 4_1-1_wobble_U-G, | | |
| | | | GATGGCAGCATTGGGATAC | 6_5-5_internal_loop- | | |
| | | | AGTGTGAAGAGCAGCA | symmetric_GCUCA-GGUGA, 17_1- | | |
| | | | | 1_mismatch_C-A, 26_6- | | |
| | | | | 6_internal_loop-symmetric_GGGGAU- | | |
| | | | | UAGGGG | | |
| 240 | 49A | 2397 (no loops) | CCCTGGTGTGCCCTCTGATG | −49_1-1_wobble_U-G, −3_1- | N/A | N/A |
| | | | TTTTTATCCCCATTCTACCGC | 1_wobble_U-G, 0_1-1_mismatch_A-C, | | |
| | | | TGTGCTGGGCAATCCCGTGG | 2_1-1_mismatch_C-C, 8_1- | | |
| | | | TCAGCAATCTTTGCAATGAT | 1_wobble_U-G, 12_1-1_wobble_U-G, | | |
| | | | GGCAGCATTGGGATACAGT | 15_1-1_mismatch_U-U, 18_1- | | |
| | | | GTGAAGAGCAGCA | 1_mismatch_U-C | | |
| 241 | 49B | 2397 (−14, +26) | CCCTGGTGTGCCCTCTGATG | −49_1-1_wobble_U-G, −14_6- | −14 | 26 |
| | | | TTTTTTAGGGGATTCTACCG | 6_internal_loop-symmetric_UGCAAA- | | |
| | | | CTGTGCTGGGCAATCCCGTG | AAACGU, −3_1-1_wobble_U-G, 0_1- | | |
| | | | GTCAGCAATCAAACGTATG | 1_mismatch_A-C, 2_1-1_mismatch_C- | | |
| | | | ATGGCAGCATTGGGATACA | C, 8_1-1_wobble_U-G, 12_1- | | |
| | | | GTGTGAAGAGCAGCA | 1_wobble_U-G, 15_1-1_mismatch_U-U, | | |
| | | | | 18_1-1_mismatch_U-C, 26_6- | | |
| | | | | 6_internal_loop-symmetric_GGGGAU- | | |
| | | | | UAGGGG | | |
| 242 | 49C | 2397 (−20, +26) | CCCTGGTGTGCCCTCTGATG | −49_1-1_wobble_U-G, −20_6- | −20 | 26 |
| | | | TTTTTTAGGGGATTCTACCG | 6_internal_loop-symmetric_CAUCAU- | | |
| | | | CTGTGCTGGGCAATCCCGTG | UACUAC, −3_1-1_wobble_U-G, 0_1- | | |
| | | | GTCAGCAATCTTTGCATACT | 1_mismatch_A-C, 2_1-1_mismatch_C- | | |
| | | | ACGCAGCATTGGGATACAG | C, 8_1-1_wobble_U-G, 12_1- | | |
| | | | TGTGAAGAGCAGCA | 1_wobble_U-G, 15_1-1_mismatch_U-U, | | |
| | | | | 18_1-1_mismatch_U-C, 26_6- | | |
| | | | | 6_internal_loop-symmetric_GGGGAU- | | |
| | | | | UAGGGG | | |
| 243 | 50A-50C | 2397 (−14, +26) 0.100.75 | AACTTCAGGTGCACGAAAC | −14_6-6_internal_loop- | −14 | 26 |
| | | | CCTGGTGTGCCCTCTGATGT | symmetric_UGCAAA-AAACGU, −3_1- | | |
| | | | TTTTTAGGGGATTCTACCGC | 1_wobble_U-G, 0_1-1_mismatch_A-C, | | |
| | | | TGTGCTGGGCAATCCCGTGG | 2_1-1_mismatch_C-C, 8_1- | | |
| | | | TCAGCAATCAAACGTATGAT | 1_wobble_U-G, 12_1-1_wobble_U-G, | | |
| | | | G | 15_1-1_mismatch_U-U, 18_1- | | |
| | | | | 1_mismatch_U-C, 26_6- | | |
| | | | | 6_internal_loop-symmetric_GGGGAU- | | |
| | | | | UAGGGG | | |
| 244 | 50A-50C | 2397 (−14, +26) 0.100.70 | CAGGTGCACGAAACCCTGG | −14_6-6_internal_loop- | −14 | 26 |
| | | | TGTGCCCTCTGATGTTTTTTA | symmetric_UGCAAA-AAACGU, −3_1- | | |
| | | | GGGGATTCTACCGCTGTGCT | 1_wobble_U-G, 0_1-1_mismatch_A-C, | | |
| | | | GGGCAATCCCGTGGTCAGC | 2_1-1_mismatch_C-C, 8_1- | | |
| | | | AATCAAACGTATGATGGCA | 1_wobble_U-G, 12_1-1_wobble_U-G, | | |
| | | | GC | 15_1-1_mismatch_U-U, 18_1- | | |
| | | | | 1_mismatch_U-C, 26_6- | | |
| | | | | 6_internal_loop-symmetric_GGGGAU- | | |
| | | | | UAGGGG | | |
| 245 | 50A-50C | 2397 (−14, +26) 0.100.65 | GCACGAAACCCTGGTGTGCC | −14_6-6_internal_loop- | −14 | 26 |
| | | | CTCTGATGTTTTTTAGGGGA | symmetric_UGCAAA-AAACGU, −3_1- | | |
| | | | TTCTACCGCTGTGCTGGGCA | 1_wobble_U-G, 0_1-1_mismatch_A-C, | | |
| | | | ATCCCGTGGTCAGCAATCAA | 2_1-1_mismatch_C-C, 8_1- | | |
| | | | ACGTATGATGGCAGCATTGG | 1_wobble_U-G, 12_1-1_wobble_U-G, | | |
| | | | | 15_1-1_mismatch_U-U, 18_1- | | |
| | | | | 1_mismatch_U-C, 26_6- | | |
| | | | | 6_internal_loop-symmetric_GGGGAU- | | |
| | | | | UAGGGG | | |
| 246 | 50A-50C | 2397 (−14, +26) 0.100.60 | AAACCCTGGTGTGCCCTCTG | −14_6-6_internal_loop- | −14 | 26 |
| | | | ATGTTTTTTAGGGGATTCTA | symmetric_UGCAAA-AAACGU, −3_1- | | |
| | | | CCGCTGTGCTGGGCAATCCC | 1_wobble_U-G, 0_1-1_mismatch_A-C, | | |
| | | | GTGGTCAGCAATCAAACGT | 2_1-1_mismatch_C-C, 8_1- | | |
| | | | ATGATGGCAGCATTGGGAT | 1_wobble_U-G, 12_1-1_wobble_U-G, | | |
| | | | AC | 15_1-1_mismatch_U-U, 18_1- | | |
| | | | | 1_mismatch_U-C, 26_6- | | |
| | | | | 6_internal_loop-symmetric_GGGGAU- | | |
| | | | | UAGGGG | | |
| 247 | 50A-50C | 2397 (−14, +26) 0.100.55 | CTGGTGTGCCCTCTGATGTT | −14_6-6_internal_loop- | −14 | 26 |
| | | | TTTTAGGGGATTCTACCGCT | symmetric_UGCAAA-AAACGU, −3_1- | | |
| | | | GTGCTGGGCAATCCCGTGGT | 1_wobble_U-G, 0_1-1_mismatch_A-C, | | |
| | | | CAGCAATCAAACGTATGAT | 2_1-1_mismatch_C-C, 8_1- | | |
| | | | GGCAGCATTGGGATACAGT | 1_wobble_U-G, 12_1-1_wobble_U-G, | | |
| | | | GT | 15_1-1_mismatch_U-U, 18_1- | | |
| | | | | 1_mismatch_U-C, 26_6- | | |
| | | | | 6_internal_loop-symmetric_GGGGAU- | | |
| | | | | UAGGGG | | |
| 248 | 50A-50C | 2397 (−14, +26) 0.100.50 | GTGCCCTCTGATGTTTTTTA | −14_6-6_internal_loop- | −14 | 26 |
| | | | GGGGATTCTACCGCTGTGCT | symmetric_UGCAAA-AAACGU, −3_1- | | |
| | | | GGGCAATCCCGTGGTCAGC | 1_wobble_U-G, 0_1-1_mismatch_A-C, | | |
| | | | AATCAAACGTATGATGGCA | 2_1-1_mismatch_C-C, 8_1- | | |
| | | | GCATTGGGATACAGTGTGA | 1_wobble_U-G, 12_1-1_wobble_U-G, | | |
| | | | AAA | 15_1-1_mismatch_U-U, 18_1- | | |
| | | | | 1_mismatch_U-C, 26_6- | | |
| | | | | 6_internal_loop-symmetric_GGGGAU- | | |
| | | | | UAGGGG | | |
| 249 | 50A-50C | 2397 (−14, +26) 0.113.57 | CCCTGGTGTGCCCTCTGATG | −49_1-1_wobble_U-G, −14_6- | −14 | 26 |
| | | | TTTTTTAGGGGATTCTACCG | 6_internal_loop-symmetric_UGCAAA- | | |
| | | | CTGTGCTGGGCAATCCCGTG | AAACGU, −3_1-1_wobble_U-G, 0_1- | | |
| | | | GTCAGCAATCAAACGTATG | 1_mismatch_A-C, 2_1-1_mismatch_C- | | |
| | | | ATGGCAGCATTGGGATACA | C, 8_1-1_wobble_U-G, 12_1- | | |
| | | | GTGTGAAGAGCAGCA | 1_wobble_U-G, 15_1-1_mismatch_U-U, | | |
| | | | | 18_1-1_mismatch_U-C, 26_6- | | |
| | | | | 6_internal_loop-symmetric_GGGGAU- | | |
| | | | | UAGGGG | | |
| 250 | 51A-51C | 2397 (−14, +30) 0.100.50 | GTGCCCTCTGATGTAAAATA | −14_6-6_internal_loop- | −14 | 30 |
| | | | CCCCATTCTACCGCTGTGCT | symmetric_UGCAAA-AAACGU, −3_1- | | |
| | | | GGGCAATCCCGTGGTCAGC | 1_wobble_U-G, 0_1-1_mismatch_A-C, | | |
| | | | AATCAAACGTATGATGGCA | 2_1-1_mismatch_C-C, 8_1- | | |
| | | | GCATTGGGATACAGTGTGA | 1_wobble_U-G, 12_1-1_wobble_U-G, | | |
| | | | AAA | 15_1-1_mismatch_U-U, 18_1- | | |
| | | | | 1_mismatch_U-C, 30_6- | | |
| | | | | 6_internal_loop-symmetric_AUAAAA- | | |
| | | | | AAAAUA | | |
| 251 | 51A-51C | 2397 (−14, +28) 0.100.50 | GTGCCCTCTGATGTTTAATA | −14_6-6_internal_loop- | −14 | 28 |
| | | | GGCCATTCTACCGCTGTGCT | symmetric_UGCAAA-AAACGU, −3_1- | | |
| | | | GGGCAATCCCGTGGTCAGC | 1_wobble_U-G, 0_1-1_mismatch_A-C, | | |
| | | | AATCAAACGTATGATGGCA | 2_1-1_mismatch_C-C, 8_1- | | |
| | | | GCATTGGGATACAGTGTGA | 1_wobble_U-G, 12_1-1_wobble_U-G, | | |
| | | | AAA | 15_1-1_mismatch_U-U, 18_1- | | |
| | | | | 1_mismatch_U-C, 28_6- | | |
| | | | | 6_internal_loop-symmetric_GGAUAA- | | |
| | | | | AAUAGG | | |
| 252 | 51A-51C | 2397 (−14, +26) 0.100.50 | GTGCCCTCTGATGTTTTTTA | −14_6-6_internal_loop- | −14 | 26 |
| | | | GGGGATTCTACCGCTGTGCT | symmetric_UGCAAA-AAACGU, −3_1- | | |
| | | | GGGCAATCCCGTGGTCAGC | 1_wobble_U-G, 0_1-1_mismatch_A-C, | | |
| | | | AATCAAACGTATGATGGCA | 2_1-1_mismatch_C-C, 8_1- | | |
| | | | GCATTGGGATACAGTGTGA | 1_wobble_U-G, 12_1-1_wobble_U-G, | | |
| | | | AAA | 15_1-1_mismatch_U-U, 18_1- | | |
| | | | | 1_mismatch_U-C, 26_6- | | |
| | | | | 6_internal_loop-symmetric_GGGGAU- | | |
| | | | | UAGGGG | | |
| 253 | 51A-51C | 2397 (−14, +25) 0.100.50 | GTGCCCTCTGATGTTTTTAA | −14_6-6_internal_loop- | −14 | 25 |
| | | | GGGGTTTCTACCGCTGTGCT | symmetric_UGCAAA-AAACGU, −3_1- | | |
| | | | GGGCAATCCCGTGGTCAGC | 1_wobble_U-G, 0_1-1_mismatch_A-C, | | |
| | | | AATCAAACGTATGATGGCA | 2_1-1_mismatch_C-C, 8_1- | | |
| | | | GCATTGGGATACAGTGTGA | 1_wobble_U-G, 12_1-1_wobble_U-G, | | |
| | | | AAA | 15_1-1_mismatch_U-U, 18_1- | | |
| | | | | 1_mismatch_U-C, 25_6- | | |
| | | | | 6_internal_loop-symmetric_UGGGGA- | | |
| | | | | AGGGGU | | |
| 254 | 51A-51C | 2397 (−14, +23) 0.100.50 | GTGCCCTCTGATGTTTTTAT | −14_6-6_internal_loop- | −14 | 23 |
| | | | CGGGTCACTACCGCTGTGCT | symmetric_UGCAAA-AAACGU, −3_1- | | |
| | | | GGGCAATCCCGTGGTCAGC | 1_wobble_U-G, 0_1-1_mismatch_A-C, | | |
| | | | AATCAAACGTATGATGGCA | 2_1-1_mismatch_C-C, 8_1- | | |
| | | | GCATTGGGATACAGTGTGA | 1_wobble_U-G, 12_1-1_wobble_U-G, | | |
| | | | AAA | 15_1-1_mismatch_U-U, 18_1- | | |
| | | | | 1_mismatch_U-C, 23_6- | | |
| | | | | 6_internal_loop-symmetric_AAUGGG- | | |
| | | | | GGGUCA | | |
| 255 | 51A-51C | 2397 (−14, +22) 0.100.50 | GTGCCCTCTGATGTTTTTAT | −14_6-6_internal_loop- | −14 | 22 |
| | | | CCGGTCAGTACCGCTGTGCT | symmetric_UGCAAA-AAACGU, −3_1- | | |
| | | | GGGCAATCCCGTGGTCAGC | 1_wobble_U-G, 0_1-1_mismatch_A-C, | | |
| | | | AATCAAACGTATGATGGCA | 2_1-1_mismatch_C-C, 8_1- | | |
| | | | GCATTGGGATACAGTGTGA | 1_wobble_U-G, 12_1-1_wobble_U-G, | | |
| | | | AAA | 15_1-1_mismatch_U-U, 18_1- | | |
| | | | | 1_mismatch_U-C, 22_6- | | |
| | | | | 6_internal_loop-symmetric_GAAUGG- | | |
| | | | | GGUCAG | | |
| 256 | 51A-51C | 2397 (−14, +21) 0.100.50 | GTGCCCTCTGATGTTTTTAT | −14_6-6_internal_loop- | −14 | 21 |
| | | | CCCGTCAGAACCGCTGTGCT | symmetric_UGCAAA-AAACGU, −3_1- | | |
| | | | GGGCAATCCCGTGGTCAGC | 1_wobble_U-G, 0_1-1_mismatch_A-C, | | |
| | | | AATCAAACGTATGATGGCA | 2_1-1_mismatch_C-C, 8_1- | | |
| | | | GCATTGGGATACAGTGTGA | 1_wobble_U-G, 12_1-1_wobble_U-G, | | |
| | | | AAA | 15_1-1_mismatch_U-U, 18_1- | | |
| | | | | 1_mismatch_U-C, 21_6- | | |
| | | | | 6_internal_loop-symmetric_AGAAUG- | | |
| | | | | GUCAGA | | |
| 257 | 52 | 2397 (−24, +28) 0.100.50 | GTGCCCTCTGATGTTTAATA | -24_6-6_internal_loop- | −24 | 28 |
| | | | GGCCATTCTACCGCTGTGCT | symmetric_CUGCCA-ACCGUC, −3_1- | | |
| | | | GGGCAATCCCGTGGTCAGC | 1_wobble_U-G, 0_1-1_mismatch_A-C, | | |
| | | | AATCTTTGCAATGAACCGTC | 2_1-1_mismatch_C-C, 8_1- | | |
| | | | CATTGGGATACAGTGTGAA | 1_wobble_U-G, 12_1-1_wobble_U-G, | | |
| | | | AA | 15_1-1_mismatch_U-U, 18_1- | | |
| | | | | 1_mismatch_U-C, 28_6- | | |
| | | | | 6_internal_loop-symmetric_GGAUAA- | | |
| | | | | AAUAGG | | |
| 258 | 52 | 2397 (−22, +28) 0.100.50 | GTGCCCTCTGATGTTTAATA | -22_6-6_internal_loop- | −22 | 28 |
| | | | GGCCATTCTACCGCTGTGCT | symmetric_GCCAUC-CUACCG, −3_1- | | |
| | | | GGGCAATCCCGTGGTCAGC | 1_wobble_U-G, 0_1-1_mismatch_A-C, | | |
| | | | AATCTTTGCAATCTACCGAG | 2_1-1_mismatch_C-C, 8_1- | | |
| | | | CATTGGGATACAGTGTGAA | 1_wobble_U-G, 12_1-1_wobble_U-G, | | |
| | | | AA | 15_1-1_mismatch_U-U, 18_1- | | |
| | | | | 1_mismatch_U-C, 28_6- | | |
| | | | | 6_internal_loop-symmetric_GGAUAA- | | |
| | | | | AAUAGG | | |
| 259 | 52 | 2397 (−20, +28) 0.100.50 | GTGCCCTCTGATGTTTAATA | -20_6-6_internal_loop- | −20 | 28 |
| | | | GGCCATTCTACCGCTGTGCT | symmetric_CAUCAU-UACUAC, −3_1- | | |
| | | | GGGCAATCCCGTGGTCAGC | 1_wobble_U-G, 0_1-1_mismatch_A-C, | | |
| | | | AATCTTTGCATACTACGCAG | 2_1-1_mismatch_C-C, 8_1- | | |
| | | | CATTGGGATACAGTGTGAA | 1_wobble_U-G, 12_1-1_wobble_U-G, | | |
| | | | AA | 15_1-1_mismatch_U-U, 18_1- | | |
| | | | | 1_mismatch_U-C, 28_6- | | |
| | | | | 6_internal_loop-symmetric_GGAUAA- | | |
| | | | | AAUAGG | | |
| 260 | 52 | 2397 (−18, +28) 0.100.50 | GTGCCCTCTGATGTTTAATA | -18_6-6_internal_loop- | −18 | 28 |
| | | | GGCCATTCTACCGCTGTGCT | symmetric_UCAUUG-GUUACU, −3_1- | | |
| | | | GGGCAATCCCGTGGTCAGC | 1_wobble_U-G, 0_1-1_mismatch_A-C, | | |
| | | | AATCTTTGGTTACTTGGCAG | 2_1-1_mismatch_C-C, 8_1- | | |
| | | | CATTGGGATACAGTGTGAA | 1_wobble_U-G, 12_1-1_wobble_U-G, | | |
| | | | AA | 15_1-1_mismatch_U-U, 18_1- | | |
| | | | | 1_mismatch_U-C, 28_6- | | |
| | | | | 6_internal_loop-symmetric_GGAUAA- | | |
| | | | | AAUAGG | | |
| 261 | 52 | 2397 (−16, +28) 0.100.50 | GTGCCCTCTGATGTTTAATA | -16_6-6_internal_loop- | −16 | 28 |
| | | | GGCCATTCTACCGCTGTGCT | symmetric_AUUGCA-ACGUUA, −3_1- | | |
| | | | GGGCAATCCCGTGGTCAGC | 1_wobble_U-G, 0_1-1_mismatch_A-C, | | |
| | | | AATCTTACGTTAGATGGCAG | 2_1-1_mismatch_C-C, 8_1- | | |
| | | | CATTGGGATACAGTGTGAA | 1_wobble_U-G, 12_1-1_wobble_U-G, | | |
| | | | AA | 15_1-1_mismatch_U-U, 18_1- | | |
| | | | | 1_mismatch_U-C, 28_6- | | |
| | | | | 6_internal_loop-symmetric_GGAUAA- | | |
| | | | | AAUAGG | | |
| 262 | 52 | 2397 (−14, +28) 0.100.50 | GTGCCCTCTGATGTTTAATA | −14_6-6_internal_loop- | −14 | 28 |
| | | | GGCCATTCTACCGCTGTGCT | symmetric_UGCAAA-AAACGU, −3_1- | | |
| | | | GGGCAATCCCGTGGTCAGC | 1_wobble_U-G, 0_1-1_mismatch_A-C, | | |
| | | | AATCAAACGTATGATGGCA | 2_1-1_mismatch_C-C, 8_1- | | |
| | | | GCATTGGGATACAGTGTGA | 1_wobble_U-G, 12_1-1_wobble_U-G, | | |
| | | | AAA | 15_1-1_mismatch_U-U, 18_1- | | |
| | | | | 1_mismatch_U-C, 28_6- | | |
| | | | | 6_internal_loop-symmetric_GGAUAA- | | |
| | | | | AAUAGG | | |
| 263 | 52 | 2397 (−12, +28) 0.100.50 | GTGCCCTCTGATGTTTAATA | -12_6-6_internal_loop- | −12 | 28 |
| | | | GGCCATTCTACCGCTGTGCT | symmetric_CAAAGA-AGAAAC, −3_1- | | |
| | | | GGGCAATCCCGTGGTCAGC | 1_wobble_U-G, 0_1-1_mismatch_A-C, | | |
| | | | AAAGAAACCAATGATGGCA | 2_1-1_mismatch_C-C, 8_1- | | |
| | | | GCATTGGGATACAGTGTGA | 1_wobble_U-G, 12_1-1_wobble_U-G, | | |
| | | | AAA | 15_1-1_mismatch_U-U, 18_1- | | |
| | | | | 1_mismatch_U-C, 28_6- | | |
| | | | | 6_internal_loop-symmetric_GGAUAA- | | |
| | | | | AAUAGG | | |
| 264 | 52 | 2397 (−10, +28) 0.100.50 | GTGCCCTCTGATGTTTAATA | -10_6-6_internal_loop- | −10 | 28 |
| | | | GGCCATTCTACCGCTGTGCT | symmetric_AAGAUU-UCAGAA, −3_1- | | |
| | | | GGGCAATCCCGTGGTCAGCT | 1_wobble_U-G, 0_1-1_mismatch_A-C, | | |
| | | | CAGAATGCAATGATGGCAG | 2_1-1_mismatch_C-C, 8_1- | | |
| | | | CATTGGGATACAGTGTGAA | 1_wobble_U-G, 12_1-1_wobble_U-G, | | |
| | | | AA | 15_1-1_mismatch_U-U, 18_1- | | |
| | | | | 1_mismatch_U-C, 28_6- | | |
| | | | | 6_internal_loop-symmetric_GGAUAA- | | |
| | | | | AAUAGG | | |
| 265 | 52 | 2397 (−9, +28) 0.100.50 | GTGCCCTCTGATGTTTAATA | -9_6-6_internal_loop- | −9 | 28 |
| | | | GGCCATTCTACCGCTGTGCT | symmetric_AGAUUG-GUCAGA, −3_1- | | |
| | | | GGGCAATCCCGTGGTCAGGT | 1_wobble_U-G, 0_1-1_mismatch_A-C, | | |
| | | | CAGATTGCAATGATGGCAG | 2_1-1_mismatch_C-C, 8_1- | | |
| | | | CATTGGGATACAGTGTGAA | 1_wobble_U-G, 12_1-1_wobble_U-G, | | |
| | | | AA | 15_1-1_mismatch_U-U, 18_1- | | |
| | | | | 1_mismatch_U-C, 28_6- | | |
| | | | | 6_internal_loop-symmetric_GGAUAA- | | |
| | | | | AAUAGG | | |
| 266 | 52 | 2397 (−7, +28) 0.100.50 | GTGCCCTCTGATGTTTAATA | −7_6-6_internal_loop- | −7 | 28 |
| | | | GGCCATTCTACCGCTGTGCT | symmetric_AUUGCU-UCGUCA, −3_1- | | |
| | | | GGGCAATCCCGTGGTCTCGT | 1_wobble_U-G, 0_1-1_mismatch_A-C, | | |
| | | | CACTTTGCAATGATGGCAGC | 2_1-1_mismatch_C-C, 8_1- | | |
| | | | ATTGGGATACAGTGTGAAA | 1_wobble_U-G, 12_1-1_wobble_U-G, | | |
| | | | A | 15_1-1_mismatch_U-U, 18_1- | | |
| | | | | 1_mismatch_U-C, 28_6- | | |
| | | | | 6_internal_loop-symmetric_GGAUAA- | | |
| | | | | AAUAGG | | |
| 267 | 53A | 1321 (no loops) | CCCTGGTGTGCCCTCTGATG | −49_1-1_wobble_U-G, −7_1- | N/A | N/A |
| | | | TTTTTATCCCCATTCTAGGG | 1_wobble_U-G, -6_1-1_mismatch_G-G, | | |
| | | | CAGTAGGGTGCACTGCCGTG | -3 1-1_wobble_U-G, 0_1- | | |
| | | | GTGGGCAATCTTTGCAATGA | 1_mismatch_A-C, 4_1-1_mismatch_U- | | |
| | | | TGGCAGCATTGGGATACAGT | C, 8_1-1_mismatch_U-U, 10_2- | | |
| | | | GTGAAGAGCAGCA | 2_bulge-symmetric_AG-GG, 18_1- | | |
| | | | | 1_wobble_U-G, 19_1-1_mismatch_G-G | | |
| 268 | 53E | 1321 (−14, +26) | CCCTGGTGTGCCCTCTGATG | −49_1-1_wobble_U-G, −14_6- | −14 | 26 |
| | | | TTTTTTAGGGGATTCTAGGG | 6_internal_loop-symmetric_UGCAAA- | | |
| | | | CAGTAGGGTGCACTGCCGTG | AAACGU, −7_1-1_wobble_U-G, -6_1- | | |
| | | | GTGGGCAATCAAACGTATG | 1_mismatch_G-G, −3_1-1_wobble_U-G, | | |
| | | | ATGGCAGCATTGGGATACA | 0_1-1_mismatch_A-C, 4_1- | | |
| | | | GTGTGAAGAGCAGCA | 1_mismatch_U-C, 8_1-1_mismatch_U- | | |
| | | | | U, 10_2-2_bulge-symmetric_AG-GG, | | |
| | | | | 18_1-1_wobble_U-G, 19_1- | | |
| | | | | 1_mismatch_G-G, 26_6- | | |
| | | | | 6_internal_loop-symmetric_GGGGAU- | | |
| | | | | UAGGGG | | |
| 269 | 54A | 295 (no loops) | CCCTGGTGTGCCCTCTGATG | -49 1-1_wobble_U-G, -4_1- | N/A | N/A |
| | | | TTTTTATCCCCATTCTACAG | 1_mismatch_C-A, −3_1-1_wobble_U-G, | | |
| | | | CAGTCCTGTACAGTGCCGTG | 0_1-1_mismatch_A-C, 4_1- | | |
| | | | ATCAGCAATCTTTGCAATGA | 1_wobble_U-G, 7_2-2_bulge- | | |
| | | | TGGCAGCATTGGGATACAGT | symmetric_CU-UA, 12_1- | | |
| | | | GTGAAGAGCAGCA | 1_mismatch_U-C | | |
| 270 | 54B | 295 (−14, +26) | CCCTGGTGTGCCCTCTGATG | −49_1-1_wobble_U-G, −14_6- | −14 | 26 |
| | | | TTTTTTAGGGGATTCTACAG | 6_internal_loop-symmetric_UGCAAA- | | |
| | | | CAGTCCTGTACAGTGCCGTG | AAACGU, -4_1-1_mismatch_C-A, | | |
| | | | ATCAGCAATCAAACGTATG | −3_1-1_wobble_U-G, 0_1- | | |
| | | | ATGGCAGCATTGGGATACA | 1_mismatch_A-C, 4_1-1_wobble_U-G, | | |
| | | | GTGTGAAGAGCAGCA | 7_2-2_bulge-symmetric_CU-UA, 12_1- | | |
| | | | | 1_mismatch_U-C, 26_6- | | |
| | | | | 6_internal_loop-symmetric_GGGGAU- | | |
| | | | | UAGGGG | | |
| 271 | 55A | 730 (no loops) | CCCTGGTGTGCCCTCTGATG | −49_1-1_wobble_U-G, −3_1- | N/A | N/A |
| | | | TTTTTATCCCCCTTCTACAGC | 1_wobble_U-G, 0_1-1_mismatch_A-C, | | |
| | | | AGTAGTGAGTTTTGCCGTGG | 4_2-2_bulge-symmetric_UU-UU, 6_1- | | |
| | | | TCAGCAATCTTTGCAATGAT | 1_wobble_G-U, 11_1-1_mismatch_G-G, | | |
| | | | GGCAGCATTGGGATACAGT | 25_1-1_mismatch_U-C | | |
| | | | GTGAAGAGCAGCA | | | |
| 272 | 55B | 730 (−14, +26) | CCCTGGTGTGCCCTCTGATG | −49_1-1_wobble_U-G, −14_6- | −14 | 25 |
| | | | TTTTTTAGGGGCTTCTACAG | 6_internal_loop-symmetric_UGCAAA- | | |
| | | | CAGTAGTGAGTTTTGCCGTG | AAACGU, −3_1-1_wobble_U-G, 0_1- | | |
| | | | GTCAGCAATCAAACGTATG | 1_mismatch_A-C, 4_2-2_bulge- | | |
| | | | ATGGCAGCATTGGGATACA | symmetric_UU-UU, 6_1-1_wobble_G- | | |
| | | | GTGTGAAGAGCAGCA | U, 11_1-1_mismatch_G-G, 25_7- | | |
| | | | | 7_internal_loop- | | |
| | | | | symmetric_UGGGGAU-UAGGGGC | | |
| 273 | 56A | 708 (no loops) | CCCTGGTGTGCCCTCTGATG | −49_1-1_wobble_U-G, −3_1- | N/A | N/A |
| | | | TTTTTATCCCCATTCTACAG | 1_wobble_U-G, 0_1-1_mismatch_A-C, | | |
| | | | CAGTACGGTGCAGTGCCGTG | 4_1-1_wobble_U-G, 8_1- | | |
| | | | GTCAGCAATCTTTGCAATGA | 1_mismatch_U-U, 10_1-1_mismatch_A- | | |
| | | | TGGCAGCATTGGGATACAGT | G | | |
| | | | GTGAAGAGCAGCA | | | |
| 274 | 56B | 708 (−14, +26) | CCCTGGTGTGCCCTCTGATG | −49_1-1_wobble_U-G, −14_6- | −14 | 26 |
| | | | TTTTTTAGGGGATTCTACAG | 6_internal_loop-symmetric_UGCAAA- | | |
| | | | CAGTACGGTGCAGTGCCGTG | AAACGU, −3_1-1_wobble_U-G, 0_1- | | |
| | | | GTCAGCAATCAAACGTATG | 1_mismatch_A-C, 4_1-1_wobble_U-G, | | |
| | | | ATGGCAGCATTGGGATACA | 8_1-1_mismatch_U-U, 10_1- | | |
| | | | GTGTGAAGAGCAGCA | 1_mismatch_A-G, 26_6- | | |
| | | | | 6_internal_loop-symmetric_GGGGAU- | | |
| | | | | UAGGGG | | |
| 275 | 57A | 351 (no loops) | CCCTGGTGTGCCCTCTGATG | −49_1-1_wobble_U-G, −3_1- | N/A | N/A |
| | | | TTTTTATCCCCATTCTACAG | 1_wobble_U-G, 0_1-1_mismatch_A-C, | | |
| | | | CGGTACTGAGCAAATCCGTG | 2_2-2_bulge-symmetric_CA-AU, 15_1- | | |
| | | | GTCAGCAATCTTTGCAATGA | 1_wobble_U-G | | |
| | | | TGGCAGCATTGGGATACAGT | | | |
| | | | GTGAAGAGCAGCA | | | |
| 276 | 57B | 351 (−14, +26) | CCCTGGTGTGCCCTCTGATG | −49_1-1_wobble_U-G, −14_6- | −14 | 26 |
| | | | TTTTTTAGGGGATTCTACAG | 6_internal_loop-symmetric_UGCAAA- | | |
| | | | CGGTACTGAGCAAATCCGTG | AAACGU, −3_1-1_wobble_U-G, 0_1- | | |
| | | | GTCAGCAATCAAACGTATG | 1_mismatch_A-C, 2_2-2_bulge- | | |
| | | | ATGGCAGCATTGGGATACA | symmetric_CA-AU, 15_1-1_wobble_U- | | |
| | | | GTGTGAAGAGCAGCA | G, 26_6-6_internal_loop- | | |
| | | | | symmetric_GGGGAU-UAGGGG | | |
| 277 | 58A | 1326 (no loops) | CCCTGGTGTGCCCTCTGATG | −49_1-1_wobble_U-G, −4_1- | N/A | N/A |
| | | | TTTTTATCCCCATTCTACACC | 1_mismatch_C-C, 0_1-1_mismatch_A- | | |
| | | | TGGACTGAGAAATCCCGTAC | C, 2_1-1_mismatch_C-C, 6_1- | | |
| | | | TCAGCAATCTTTGCAATGAT | 1_mismatch_G-A, 13_3−3_bulge- | | |
| | | | GGCAGCATTGGGATACAGT | symmetric_ACU-UGG, 17_1- | | |
| | | | GTGAAGAGCAGCA | 1_mismatch_C-C | | |
| 278 | 58B | 1326 (−14, +26) | CCCTGGTGTGCCCTCTGATG | −49_1-1_wobble_U-G, −14_6- | −14 | 26 |
| | | | TTTTTTAGGGGATTCTACAC | 6_internal_loop-symmetric_UGCAAA- | | |
| | | | CTGGACTGAGAAATCCCGTA | AAACGU, −4_1-1_mismatch_C-C, 0_1- | | |
| | | | CTCAGCAATCAAACGTATGA | 1_mismatch_A-C, 2_1-1_mismatch_C- | | |
| | | | TGGCAGCATTGGGATACAGT | C, 6_1-1_mismatch_G-A, 13_3- | | |
| | | | GTGAAGAGCAGCA | 3_bulge-symmetric_ACU-UGG, 17_1- | | |
| | | | | 1_mismatch_C-C, 26_6- | | |
| | | | | 6_internal_loop-symmetric_GGGGAU- | | |
| | | | | UAGGGG | | |
| 279 | 59A-59B | 871 (−22, +26) | CCCTGGTGTGCCCTCTGATG | −49_1-1_wobble_U-G, -22_6- | −22 | 26 |
| | | | TTTTTCGAAGAATTCTACAG | 6_internal_loop-symmetric_GCCAUC- | | |
| | | | TAGGACTGAGCACTGCCGA | CAAAAA, −7_1-1_wobble_U-G, -6_1- | | |
| | | | GCTGGGCAATCTTTGCAATC | 1_mismatch_G-G, -4_0-1_bulge- | | |
| | | | AAAAAAGCATTGGGATACA | asymmetric_-C, -2_1-0_bulge- | | |
| | | | GTGTGAAGAGCAGCA | asymmetric_A-, 0_1-1_mismatch_A-C, | | |
| | | | | 4_1-1_mismatch_U-C, 13_1- | | |
| | | | | 1_mismatch_A-G, 16_1-1_wobble_G-U, | | |
| | | | | 26_6-6_internal_loop- | | |
| | | | | symmetric_GGGGAU-CGAAGA | | |
| 280 | 59C-59D | 871 (−16, +22) | CCCTGGTGTGCCCTCTGATG | −49_1-1_wobble_U-G, -16_6- | −16 | 22 |
| | | | TTTTTATCCGAAACATACAG | 6_internal_loop-symmetric_AUUGCA- | | |
| | | | TAGGACTGAGCACTGCCGA | CUCCCA, −7_1-1_wobble_U-G, -6_1- | | |
| | | | GCTGGGCAATCTTCTCCCAG | 1_mismatch_G-G, -4_0-1_bulge- | | |
| | | | ATGGCAGCATTGGGATACA | asymmetric_-C, -2_1-0_bulge- | | |
| | | | GTGTGAAGAGCAGCA | asymmetric_A-, 0_1-1_mismatch_A-C, | | |
| | | | | 4_1-1_mismatch_U-C, 13_1- | | |
| | | | | 1_mismatch_A-G, 16_1-1_wobble_G-U, | | |
| | | | | 22_6-6_internal_loop- | | |
| | | | | symmetric_GAAUGG-GAAACA | | |
| 281 | 59E-59F | 871 (−8, +26) | CCCTGGTGTGCCCTCTGATG | −49_1-1_wobble_U-G, -9_4-4_bulge- | N/A | 26 |
| | | | TTTTTTACAGAATTCTACAG | symmetric_AUUG-GUUC, −7_1- | | |
| | | | TAGGACTGAGCACTGCCGA | 1_wobble_U-G, -6_1-1_mismatch_G-G, | | |
| | | | GCTGGGGTTCCTTTGCAATG | -4_0-1_bulge-asymmetric_-C, -2_1- | | |
| | | | ATGGCAGCATTGGGATACA | 0_bulge-asymmetric_A-, 0_1- | | |
| | | | GTGTGAAGAGCAGCA | 1_mismatch_A-C, 4_1-1_mismatch_U- | | |
| | | | | C, 13_1-1_mismatch_A-G, 16_1- | | |
| | | | | 1_wobble_G-U, 26_6-6_internal_loop- | | |
| | | | | symmetric_GGGGAU-UACAGA | | |
| 23 | 59G-59I | 871 (−16, +24) | CCCTGGTGTGCCCTCTGATG | −49_1-1_wobble_U-G, -16_6- | −16 | 24 |
| | | | TTTTTATGGGGTGTCTACAG | 6_internal_loop-symmetric_AUUGCA- | | |
| | | | TAGGACTGAGCACTGCCGA | GAGUCG, −7_1-1_wobble_U-G, -6_1- | | |
| | | | GCTGGGCAATCTTGAGTCGG | 1_mismatch_G-G, -4_0-1_bulge- | | |
| | | | ATGGCAGCATTGGGATACA | asymmetric_-C, -2_1-0_bulge- | | |
| | | | GTGTGAAGAGCAGCA | asymmetric_A-, 0_1-1_mismatch_A-C, | | |
| | | | | 4_1-1_mismatch_U-C, 13_1- | | |
| | | | | 1_mismatch_A-G, 16_1-1_wobble_G-U, | | |
| | | | | 24_6-6_internal_loop- | | |
| | | | | symmetric_AUGGGG-GGGGUG | | |
| 282 | 59J-59K | 871 (−10, +26) | CCCTGGTGTGCCCTCTGATG | −49_1-1_wobble_U-G, -10_6- | −10 | 26 |
| | | | TTTTTCATGAGATTCTACAG | 6_internal_loop-symmetric_AAGAUU- | | |
| | | | TAGGACTGAGCACTGCCGA | CUCAGG, −7_1-1_wobble_U-G, -6_1- | | |
| | | | GCTGGGCCTCAGGTGCAATG | 1_mismatch_G-G, -4_0-1_bulge- | | |
| | | | ATGGCAGCATTGGGATACA | asymmetric_-C, -2_1-0_bulge- | | |
| | | | GTGTGAAGAGCAGCA | asymmetric_A-, 0_1-1_mismatch_A-C, | | |
| | | | | 4_1-1_mismatch_U-C, 13_1- | | |
| | | | | 1_mismatch_A-G, 16_1-1_wobble_G-U, | | |
| | | | | 26_6-6_internal_loop- | | |
| | | | | symmetric_GGGGAU-CAUGAG | | |
| 283 | 59L-59M | 871 (−16, +26) | CCCTGGTGTGCCCTCTGATG | −49_1-1_wobble_U-G, -16_6- | −16 | N/A |
| | | | TTTTTCTACAGATTCTACAG | 6_internal_loop-symmetric_AUUGCA- | | |
| | | | TAGGACTGAGCACTGCCGA | CAUUUG, −7_1-1_wobble_U-G, -6_1- | | |
| | | | GCTGGGCAATCTTCATTTGG | 1_mismatch_G-G, -4_0-1_bulge- | | |
| | | | ATGGCAGCATTGGGATACA | asymmetric_-C, -2_1-0_bulge- | | |
| | | | GTGTGAAGAGCAGCA | asymmetric_A-, 0_1-1_mismatch_A-C, | | |
| | | | | 4_1-1_mismatch_U-C, 13_1- | | |
| | | | | 1_mismatch_A-G, 16_1-1_wobble_G-U, | | |
| | | | | 26_2-2_bulge-symmetric_GG-AG, | | |
| | | | | 29_3−3_bulge-symmetric_GAU-CUA | | |
| 284 | 59N-590 | 871 (−20, +26) | CCCTGGTGTGCCCTCTGATG | −49_1-1_wobble_U-G, -20_6- | −20 | N/A |
| | | | TTCTTTAGGGGATTCTACAG | 6_internal_loop-symmetric_CAUCAU- | | |
| | | | TAGGACTGAGCACTGCCGA | UACUAC, −7_1-1_wobble_U-G, -6_1- | | |
| | | | GCTGGGCAATCTTTGCATAC | 1_mismatch_G-G, -4_0-1_bulge- | | |
| | | | TACGCAGCATTGGGATACA | asymmetric_-C, -2_1-0_bulge- | | |
| | | | GTGTGAAGAGCAGCA | asymmetric_A-, 0_1-1_mismatch_A-C, | | |
| | | | | 4_1-1_mismatch_U-C, 13_1- | | |
| | | | | 1_mismatch_A-G, 16_1-1_wobble_G-U, | | |
| | | | | 26_5-4_internal_loop- | | |
| | | | | asymmetric_GGGGA-GGGG, 34_0- | | |
| | | | | 1_bulge-asymmetric_-C | | |
| 285 | 60A-60C | 871 (−16, +24) 0.100.75 | AACTTCAGGTGCACGAAAC | -16_6-6_internal_loop- | −16 | 24 |
| | | | CCTGGTGTGCCCTCTGATGT | symmetric_AUUGCA-GAGUCG, −7_1- | | |
| | | | TTTTATGGGGTGTCTACAGT | 1_wobble_U-G, -6_1-1_mismatch_G-G, | | |
| | | | AGGACTGAGCACTGCCGAG | -4_0-1_bulge-asymmetric_-C, -2_1- | | |
| | | | CTGGGCAATCTTGAGTCGGA | 0_bulge-asymmetric_A-, 0_1- | | |
| | | | TG | 1_mismatch_A-C, 4_1-1_mismatch_U- | | |
| | | | | C, 13_1-1_mismatch_A-G, 16_1- | | |
| | | | | 1_wobble_G-U, 24_6-6_internal_loop- | | |
| | | | | symmetric_AUGGGG-GGGGUG | | |
| 286 | 60A-60C | 871 (−16, +24) 0.100.70 | CAGGTGCACGAAACCCTGG | -16_6-6_internal_loop- | −16 | 24 |
| | | | TGTGCCCTCTGATGTTTTTAT | symmetric_AUUGCA-GAGUCG, −7_1- | | |
| | | | GGGGTGTCTACAGTAGGACT | 1_wobble_U-G, -6_1-1_mismatch_G-G, | | |
| | | | GAGCACTGCCGAGCTGGGC | -4_0-1_bulge-asymmetric_-C, -2_1- | | |
| | | | AATCTTGAGTCGGATGGCAG | 0_bulge-asymmetric_A-, 0_1- | | |
| | | | C | 1_mismatch_A-C, 4_1-1_mismatch_U- | | |
| | | | | C, 13_1-1_mismatch_A-G, 16_1- | | |
| | | | | 1_wobble_G-U, 24_6-6_internal_loop- | | |
| | | | | symmetric_AUGGGG-GGGGUG | | |
| 287 | 60A-60C | 871 (−16, +24) 0.100.65 | GCACGAAACCCTGGTGTGCC | -16_6-6_internal_loop- | −16 | 24 |
| | | | CTCTGATGTTTTTATGGGGT | symmetric_AUUGCA-GAGUCG, −7_1- | | |
| | | | GTCTACAGTAGGACTGAGC | 1_wobble_U-G, -6_1-1_mismatch_G-G, | | |
| | | | ACTGCCGAGCTGGGCAATCT | -4_0-1_bulge-asymmetric_-C, -2_1- | | |
| | | | TGAGTCGGATGGCAGCATTG | 0_bulge-asymmetric_A-, 0_1- | | |
| | | | G | 1_mismatch_A-C, 4_1-1_mismatch_U- | | |
| | | | | C, 13_1-1_mismatch_A-G, 16_1- | | |
| | | | | 1_wobble_G-U, 24_6-6_internal_loop- | | |
| | | | | symmetric_AUGGGG-GGGGUG | | |
| 288 | 60A-60C | 871 (−16, +24) 0.100.60 | AAACCCTGGTGTGCCCTCTG | -16_6-6_internal_loop- | −16 | 24 |
| | | | ATGTTTTTATGGGGTGTCTA | symmetric_AUUGCA-GAGUCG, −7_1- | | |
| | | | CAGTAGGACTGAGCACTGC | 1_wobble_U-G, -6_1-1_mismatch_G-G, | | |
| | | | CGAGCTGGGCAATCTTGAGT | -4_0-1_bulge-asymmetric_-C, -2_1- | | |
| | | | CGGATGGCAGCATTGGGAT | 0_bulge-asymmetric_A-, 0_1- | | |
| | | | AC | 1_mismatch_A-C, 4_1-1_mismatch_U- | | |
| | | | | C, 13_1-1_mismatch_A-G, 16_1- | | |
| | | | | 1_wobble_G-U, 24_6-6_internal_loop- | | |
| | | | | symmetric_AUGGGG-GGGGUG | | |
| 289 | 60A-60C | 871 (−16, +24) 0.100.55 | CTGGTGTGCCCTCTGATGTT | -16_6-6_internal_loop- | −16 | 24 |
| | | | TTTATGGGGTGTCTACAGTA | symmetric_AUUGCA-GAGUCG, −7_1- | | |
| | | | GGACTGAGCACTGCCGAGC | 1_wobble_U-G, -6_1-1_mismatch_G-G, | | |
| | | | TGGGCAATCTTGAGTCGGAT | -4_0-1_bulge-asymmetric_-C, -2_1- | | |
| | | | GGCAGCATTGGGATACAGT | 0_bulge-asymmetric_A-, 0_1- | | |
| | | | GT | 1_mismatch_A-C, 4_1-1_mismatch_U- | | |
| | | | | C, 13_1-1_mismatch_A-G, 16_1- | | |
| | | | | 1_wobble_G-U, 24_6-6_internal_loop- | | |
| | | | | symmetric_AUGGGG-GGGGUG | | |
| 290 | 60A-60C | 871 (−16, +24) 0.100.50 | GTGCCCTCTGATGTTTTTAT | -16_6-6_internal_loop- | −16 | 24 |
| | | | GGGGTGTCTACAGTAGGACT | symmetric_AUUGCA-GAGUCG, −7_1- | | |
| | | | GAGCACTGCCGAGCTGGGC | 1_wobble_U-G, -6_1-1_mismatch_G-G, | | |
| | | | AATCTTGAGTCGGATGGCAG | -4_0-1_bulge-asymmetric_-C, -2_1- | | |
| | | | CATTGGGATACAGTGTGAA | 0_bulge-asymmetric_A-, 0_1- | | |
| | | | AA | 1_mismatch_A-C, 4_1-1_mismatch_U- | | |
| | | | | C, 13_1-1_mismatch_A-G, 16_1- | | |
| | | | | 1_wobble_G-U, 24_6-6_internal_loop- | | |
| | | | | symmetric_AUGGGG-GGGGUG | | |
| 23 | 60A-60C | 871 (−16, +24) 0.113.57 | CCCTGGTGTGCCCTCTGATG | −49_1-1_wobble_U-G, -16_6- | −16 | 24 |
| | | | TTTTTATGGGGTGTCTACAG | 6_internal_loop-symmetric_AUUGCA- | | |
| | | | TAGGACTGAGCACTGCCGA | GAGUCG, −7_1-1_wobble_U-G, -6_1- | | |
| | | | GCTGGGCAATCTTGAGTCGG | 1_mismatch_G-G, -4_0-1_bulge- | | |
| | | | ATGGCAGCATTGGGATACA | asymmetric_-C, -2_1-0_bulge- | | |
| | | | GTGTGAAGAGCAGCA | asymmetric_A-, 0_1-1_mismatch_A-C, | | |
| | | | | 4_1-1_mismatch_U-C, 13_1- | | |
| | | | | 1_mismatch_A-G, 16_1-1_wobble_G-U, | | |
| | | | | 24_6-6_internal_loop- | | |
| | | | | symmetric_AUGGGG-GGGGUG | | |
| 291 | 61A-61C | 871 (−16, +34) 0.100.55 | CTGGTGTGCCCTCTGTACCA | -16_6-6_internal_loop- | −16 | 34 |
| | | | ATTATCCCCATTCTACAGTA | symmetric_AUUGCA-GAGUCG, −7_1- | | |
| | | | GGACTGAGCACTGCCGAGC | 1_wobble_U-G, -6_1-1_mismatch_G-G, | | |
| | | | TGGGCAATCTTGAGTCGGAT | -4_0-1_bulge-asymmetric_-C, -2_1- | | |
| | | | GGCAGCATTGGGATACAGT | 0_bulge-asymmetric_A-, 0_1- | | |
| | | | GT | 1_mismatch_A-C, 4_1-1_mismatch_U- | | |
| | | | | C, 13_1-1_mismatch_A-G, 16_1- | | |
| | | | | 1_wobble_G-U, 34_6-6_internal_loop- | | |
| | | | | symmetric_AAACAU-UACCAA | | |
| 292 | 61A-61C | 871 (−16, +32) 0.100.55 | CTGGTGTGCCCTCTGATCCA | -16_6-6_internal_loop- | −16 | 32 |
| | | | AGCATCCCCATTCTACAGTA | symmetric_AUUGCA-GAGUCG, −7_1- | | |
| | | | GGACTGAGCACTGCCGAGC | 1_wobble_U-G, -6_1-1_mismatch_G-G, | | |
| | | | TGGGCAATCTTGAGTCGGAT | -4_0-1_bulge-asymmetric_-C, -2_1- | | |
| | | | GGCAGCATTGGGATACAGT | 0_bulge-asymmetric_A-, 0_1- | | |
| | | | GT | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 293 | 61A-61C | 871 (−16, +30) 0.100.55 | CTGGTGTGCCCTCTGATGTA | -16_6-6_internal_loop- | −16 | 30 |
| | | | AGCTACCCCATTCTACAGTA | symmetric_AUUGCA-GAGUCG, −7_1- | | |
| | | | GGACTGAGCACTGCCGAGC | 1_wobble_U-G, -6_1-1_mismatch_G-G, | | |
| | | | TGGGCAATCTTGAGTCGGAT | -4_0-1_bulge-asymmetric_-C, -2_1- | | |
| | | | GGCAGCATTGGGATACAGT | 0_bulge-asymmetric_A-, 0_1- | | |
| | | | GT | 1_mismatch_A-C, 4_1-1_mismatch_U- | | |
| | | | | C, 13_1-1_mismatch_A-G, 16_1- | | |
| | | | | 1_wobble_G-U, 30_6-6_internal_loop- | | |
| | | | | symmetric_AUAAAA-AAGCUA | | |
| 294 | 61A-61C | 871 (−16, +28) 0.100.55 | CTGGTGTGCCCTCTGATGTT | -16_6-6_internal_loop- | −16 | 28 |
| | | | TGCTAGGCCATTCTACAGTA | symmetric_AUUGCA-GAGUCG, −7_1- | | |
| | | | GGACTGAGCACTGCCGAGC | 1_wobble_U-G, -6_1-1_mismatch_G-G, | | |
| | | | TGGGCAATCTTGAGTCGGAT | -4_0-1_bulge-asymmetric_-C, -2_1- | | |
| | | | GGCAGCATTGGGATACAGT | 0_bulge-asymmetric_A-, 0_1- | | |
| | | | GT | 1_mismatch_A-C, 4_1-1_mismatch_U- | | |
| | | | | C, 13_1-1_mismatch_A-G, 16_1- | | |
| | | | | 1_wobble_G-U, 28_6-6_internal_loop- | | |
| | | | | symmetric_GGAUAA-GCUAGG | | |
| 295 | 61A-61C | 871 (−16, +26) 0.100.55 | CTGGTGTGCCCTCTGATGTT | -16_6-6_internal_loop- | −16 | 26 |
| | | | TTTTAGGGGATTCTACAGTA | symmetric_AUUGCA-GAGUCG, −7_1- | | |
| | | | GGACTGAGCACTGCCGAGC | 1_wobble_U-G, -6_1-1_mismatch_G-G, | | |
| | | | TGGGCAATCTTGAGTCGGAT | -4_0-1_bulge-asymmetric_-C, -2_1- | | |
| | | | GGCAGCATTGGGATACAGT | 0_bulge-asymmetric_A-, 0_1- | | |
| | | | GT | 1_mismatch_A-C, 4_1-1_mismatch_U- | | |
| | | | | C, 13_1-1_mismatch_A-G, 16_1- | | |
| | | | | 1_wobble_G-U, 26_6-6_internal_loop- | | |
| | | | | symmetric_GGGGAU-UAGGGG | | |
| 296 | 61A-61C | 871 (−16, +24) 0.100.55 | CTGGTGTGCCCTCTGATGTT | -16_6-6_internal_loop- | −16 | 24 |
| | | | TTTATGGGGTGTCTACAGTA | symmetric_AUUGCA-GAGUCG, −7_1- | | |
| | | | GGACTGAGCACTGCCGAGC | 1_wobble_U-G, -6_1-1_mismatch_G-G, | | |
| | | | TGGGCAATCTTGAGTCGGAT | -4_0-1_bulge-asymmetric_-C, -2_1- | | |
| | | | GGCAGCATTGGGATACAGT | 0_bulge-asymmetric_A-, 0_1- | | |
| | | | GT | 1_mismatch_A-C, 4_1-1_mismatch_U- | | |
| | | | | C, 13_1-1_mismatch_A-G, 16_1- | | |
| | | | | 1_wobble_G-U, 24_6-6_internal_loop- | | |
| | | | | symmetric_AUGGGG-GGGGUG | | |
| 297 | 61A-61C | 871 (−16, +22) 0.100.55 | CTGGTGTGCCCTCTGATGTT | -16_6-6_internal_loop- | −16 | 22 |
| | | | TTTATCCGGTGAGTACAGTA | symmetric_AUUGCA-GAGUCG, −7_1- | | |
| | | | GGACTGAGCACTGCCGAGC | 1_wobble_U-G, -6_1-1_mismatch_G-G, | | |
| | | | TGGGCAATCTTGAGTCGGAT | -4_0-1_bulge-asymmetric_-C, -2_1- | | |
| | | | GGCAGCATTGGGATACAGT | 0_bulge-asymmetric_A-, 0_1- | | |
| | | | GT | 1_mismatch_A-C, 4_1-1_mismatch_U- | | |
| | | | | C, 13_1-1_mismatch_A-G, 16 1- | | |
| | | | | 1_wobble_G-U, 22_6-6_internal_loop- | | |
| | | | | symmetric_GAAUGG-GGUGAG | | |
| 298 | 61A-61C | 871 (−16, +20) 0.100.55 | CTGGTGTGCCCTCTGATGTT | -16_6-6_internal_loop- | −16 | 20 |
| | | | TTTATCCCCTGAGATCAGTA | symmetric_AUUGCA-GAGUCG, −7_1- | | |
| | | | GGACTGAGCACTGCCGAGC | 1_wobble_U-G, -6_1-1_mismatch_G-G, | | |
| | | | TGGGCAATCTTGAGTCGGAT | -4_0-1_bulge-asymmetric_-C, -2_1- | | |
| | | | GGCAGCATTGGGATACAGT | 0_bulge-asymmetric_A-, 0_1- | | |
| | | | GT | 1_mismatch_A-C, 4_1-1_mismatch_U- | | |
| | | | | C, 13_1-1_mismatch_A-G, 16_1- | | |
| | | | | 1_wobble_G-U, 20_6-6_internal_loop- | | |
| | | | | symmetric_UAGAAU-UGAGAU | | |
| 299 | 61A-61C | 871 (−16, +18) 0.100.55 | CTGGTGTGCCCTCTGATGTT | -16_6-6_internal_loop- | −16 | 18 |
| | | | TTTATCCCCATAGATGTGTA | symmetric_AUUGCA-GAGUCG, −7_1- | | |
| | | | GGACTGAGCACTGCCGAGC | 1_wobble_U-G, -6_1-1_mismatch_G-G, | | |
| | | | TGGGCAATCTTGAGTCGGAT | -4_0-1_bulge-asymmetric_-C, -2_1- | | |
| | | | GGCAGCATTGGGATACAGT | 0_bulge-asymmetric_A-, 0_1- | | |
| | | | GT | 1_mismatch_A-C, 4_1-1_mismatch_U- | | |
| | | | | C, 13_1-1_mismatch_A-G, 16_1- | | |
| | | | | 1_wobble_G-U, 18_6-6_internal_loop- | | |
| | | | | symmetric_UGUAGA-AGAUGU | | |
| 300 | 62A-62C | 871 (−30, +32) 0.100.55 | CTGGTGTGCCCTCTGATCCA | -30_6-6_internal_loop- | −30 | 32 |
| | | | AGCATCCCCATTCTACAGTA | symmetric_CCAAUG-GUGACC, −7_1- | | |
| | | | GGACTGAGCACTGCCGAGC | 1_wobble_U-G, -6_1-1_mismatch_G-G, | | |
| | | | TGGGCAATCTTTGCAATGAT | -4_0-1_bulge-asymmetric_-C, -2_1- | | |
| | | | GGCAGGTGACCGATACAGT | 0_bulge-asymmetric_A-, 0_1- | | |
| | | | GT | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 301 | 62A-62C | 871 (−27, +32) 0.100.55 | CTGGTGTGCCCTCTGATCCA | -27_6-6_internal_loop- | −27 | 32 |
| | | | AGCATCCCCATTCTACAGTA | symmetric_AUGCUG-GUCGUG, −7_1- | | |
| | | | GGACTGAGCACTGCCGAGC | 1_wobble_U-G, -6_1-1_mismatch_G-G, | | |
| | | | TGGGCAATCTTTGCAATGAT | -4_0-1_bulge-asymmetric_-C, -2_1- | | |
| | | | GGGTCGTGTGGGATACAGT | 0_bulge-asymmetric_A-, 0_1- | | |
| | | | GT | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 302 | 62A-62C | 871 (−24, +32) 0.100.55 | CTGGTGTGCCCTCTGATCCA | -24_6-6_internal_loop- | −24 | 32 |
| | | | AGCATCCCCATTCTACAGTA | symmetric_CUGCCA-ACCGUC, −7_1- | | |
| | | | GGACTGAGCACTGCCGAGC | 1_wobble_U-G, -6_1-1_mismatch_G-G, | | |
| | | | TGGGCAATCTTTGCAATGAA | -4_0-1_bulge-asymmetric_-C, -2_1- | | |
| | | | CCGTCCATTGGGATACAGTG | 0_bulge-asymmetric_A-, 0_1- | | |
| | | | T | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 303 | 62A-62C | 871 (−22, +32) 0.100.55 | CTGGTGTGCCCTCTGATCCA | -22_6-6_internal_loop- | −22 | 32 |
| | | | AGCATCCCCATTCTACAGTA | symmetric_GCCAUC-CUACCG, −7_1- | | |
| | | | GGACTGAGCACTGCCGAGC | 1_wobble_U-G, -6_1-1_mismatch_G-G, | | |
| | | | TGGGCAATCTTTGCAATCTA | -4_0-1_bulge-asymmetric_-C, -2_1- | | |
| | | | CCGAGCATTGGGATACAGT | 0_bulge-asymmetric_A-, 0_1- | | |
| | | | GT | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 304 | 62A-62C | 871 (−20, +32) 0.100.55 | CTGGTGTGCCCTCTGATCCA | -20_6-6_internal_loop- | −20 | 32 |
| | | | AGCATCCCCATTCTACAGTA | symmetric_CAUCAU-CGCUAC, −7_1- | | |
| | | | GGACTGAGCACTGCCGAGC | 1_wobble_U-G, -6_1-1_mismatch_G-G, | | |
| | | | TGGGCAATCTTTGCACGCTA | -4_0-1_bulge-asymmetric_-C, -2_1- | | |
| | | | CGCAGCATTGGGATACAGT | 0_bulge-asymmetric_A-, 0_1- | | |
| | | | GT | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 305 | 62A-62C | 871 (−18, +32) 0.100.55 | CTGGTGTGCCCTCTGATCCA | -18_6-6_internal_loop- | −18 | 32 |
| | | | AGCATCCCCATTCTACAGTA | symmetric_UCAUUG-GUCGCU, −7_1- | | |
| | | | GGACTGAGCACTGCCGAGC | 1_wobble_U-G, -6_1-1_mismatch_G-G, | | |
| | | | TGGGCAATCTTTGGTCGCTT | -4_0-1_bulge-asymmetric_-C, -2_1- | | |
| | | | GGCAGCATTGGGATACAGT | 0_bulge-asymmetric_A-, 0_1- | | |
| | | | GT | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 306 | 62A-62C | 871 (−16, +32) 0.100.55 | CTGGTGTGCCCTCTGATCCA | -16_6-6_internal_loop- | −16 | 32 |
| | | | AGCATCCCCATTCTACAGTA | symmetric_AUUGCA-GAGUCG, −7_1- | | |
| | | | GGACTGAGCACTGCCGAGC | 1_wobble_U-G, -6_1-1_mismatch_G-G, | | |
| | | | TGGGCAATCTTGAGTCGGAT | -4_0-1_bulge-asymmetric_-C, -2_1- | | |
| | | | GGCAGCATTGGGATACAGT | 0_bulge-asymmetric_A-, 0_1- | | |
| | | | GT | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 307 | 62A-62C | 871 (−13, +32) 0.100.55 | CTGGTGTGCCCTCTGATCCA | -13_6-6_internal_loop- | −13 | 32 |
| | | | AGCATCCCCATTCTACAGTA | symmetric_GCAAAG-GAAGAG, −7_1- | | |
| | | | GGACTGAGCACTGCCGAGC | 1_wobble_U-G, -6_1-1_mismatch_G-G, | | |
| | | | TGGGCAATGAAGAGAATGA | -4_0-1_bulge-asymmetric_-C, -2_1- | | |
| | | | TGGCAGCATTGGGATACAGT | 0_bulge-asymmetric_A-, 0_1- | | |
| | | | GT | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 308 | 63A-63C | 871 (−16, +32) | CTGGTGTGCCCTCTGATCCA | -16_6-6_internal_loop- | −16 | 32 |
| | | 0.90.55L10 | AGCATCCCCATTCTACAGTA | symmetric_AUUGCA-GAGUCG, −7_1- | | |
| | | | GGACTGAGCACTGCCGAGC | 1_wobble_U-G, -6_1-1_mismatch_G-G, | | |
| | | | TGGGCAATCTTGAGTCGGAT | -4_0-1_bulge-asymmetric_-C, -2_1- | | |
| | | | GGCAGCATTGG | 0_bulge-asymmetric_A-, 0_1- | | |
| | | | | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 309 | 63A-63C | 871 (−16, +32) 0.92.55L8 | CTGGTGTGCCCTCTGATCCA | -16_6-6_internal_loop- | −16 | 32 |
| | | | AGCATCCCCATTCTACAGTA | symmetric_AUUGCA-GAGUCG, −7_1- | | |
| | | | GGACTGAGCACTGCCGAGC | 1_wobble_U-G, -6_1-1_mismatch_G-G, | | |
| | | | TGGGCAATCTTGAGTCGGAT | -4_0-1_bulge-asymmetric_-C, -2_1- | | |
| | | | GGCAGCATTGGGA | 0_bulge-asymmetric_A-, 0_1- | | |
| | | | | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 310 | 63A-63C | 871 (−16, +32) 0.94.55L6 | CTGGTGTGCCCTCTGATCCA | -16_6-6_internal_loop- | −16 | 32 |
| | | | AGCATCCCCATTCTACAGTA | symmetric_AUUGCA-GAGUCG, −7_1- | | |
| | | | GGACTGAGCACTGCCGAGC | 1_wobble_U-G, -6_1-1_mismatch_G-G, | | |
| | | | TGGGCAATCTTGAGTCGGAT | -4_0-1_bulge-asymmetric_-C, -2_1- | | |
| | | | GGCAGCATTGGGATA | 0_bulge-asymmetric_A-, 0_1- | | |
| | | | | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 311 | 63A-63C | 871 (−16, +32) 0.96.55L4 | CTGGTGTGCCCTCTGATCCA | -16_6-6_internal_loop- | −16 | 32 |
| | | | AGCATCCCCATTCTACAGTA | symmetric_AUUGCA-GAGUCG, −7_1- | | |
| | | | GGACTGAGCACTGCCGAGC | 1_wobble_U-G, -6_1-1_mismatch_G-G, | | |
| | | | | -4_0-1_bulge-asymmetric_-C, -2_1- | | |
| | | | TGGGCAATCTTGAGTCGGAT | 0_bulge-asymmetric_A-, 0_1- | | |
| | | | GGCAGCATTGGGATACA | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 312 | 63A-63C | 871 (−16, +32) 0.98.55L2 | CTGGTGTGCCCTCTGATCCA | -16_6-6_internal_loop- | −16 | 32 |
| | | | AGCATCCCCATTCTACAGTA | symmetric_AUUGCA-GAGUCG, −7_1- | | |
| | | | GGACTGAGCACTGCCGAGC | 1_wobble_U-G, -6_1-1_mismatch_G-G, | | |
| | | | TGGGCAATCTTGAGTCGGAT | -4_0-1_bulge-asymmetric_-C, -2_1- | | |
| | | | GGCAGCATTGGGATACAGT | 0_bulge-asymmetric_A-, 0_1- | | |
| | | | | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 313 | 63A-63C | 871 (−16, +32) | CTCTGATCCAAGCATCCCCA | -16_6-6_internal_loop- | −16 | 32 |
| | | 0.90.45R10 | TTCTACAGTAGGACTGAGCA | symmetric_AUUGCA-GAGUCG, −7_1- | | |
| | | | CTGCCGAGCTGGGCAATCTT | 1_wobble_U-G, -6_1-1_mismatch_G-G, | | |
| | | | GAGTCGGATGGCAGCATTG | -4_0-1_bulge-asymmetric_-C, -2_1- | | |
| | | | GGATACAGTGT | 0_bulge-asymmetric_A-, 0_1- | | |
| | | | | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 314 | 63A-63C | 871 (−16, +32) 0.92.47R8 | CCCTCTGATCCAAGCATCCC | -16_6-6_internal_loop- | −16 | 32 |
| | | | CATTCTACAGTAGGACTGAG | symmetric_AUUGCA-GAGUCG, −7_1- | | |
| | | | CACTGCCGAGCTGGGCAATC | 1_wobble_U-G, -6_1-1_mismatch_G-G, | | |
| | | | TTGAGTCGGATGGCAGCATT | -4_0-1_bulge-asymmetric_-C, -2_1- | | |
| | | | GGGATACAGTGT | 0_bulge-asymmetric_A-, 0_1- | | |
| | | | | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 315 | 63A-63C | 871 (−16, +32) 0.94.49R6 | TGCCCTCTGATCCAAGCATC | -16_6-6_internal_loop- | −16 | 32 |
| | | | CCCATTCTACAGTAGGACTG | symmetric_AUUGCA-GAGUCG, -7_1- | | |
| | | | AGCACTGCCGAGCTGGGCA | 1_wobble_U-G, -6_1-1_mismatch_G-G, | | |
| | | | ATCTTGAGTCGGATGGCAGC | -4_0-1_bulge-asymmetric_-C, -2_1- | | |
| | | | ATTGGGATACAGTGT | 0_bulge-asymmetric_A-, 0_1- | | |
| | | | | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 316 | 63A-63C | 871 (−16, +32) 0.96.51R4 | TGTGCCCTCTGATCCAAGCA | -16_6-6_internal_loop- | −16 | 32 |
| | | | TCCCCATTCTACAGTAGGAC | symmetric_AUUGCA-GAGUCG, −7_1- | | |
| | | | TGAGCACTGCCGAGCTGGG | 1_wobble_U-G, -6_1-1_mismatch_G-G, | | |
| | | | CAATCTTGAGTCGGATGGCA | -4_0-1_bulge-asymmetric_-C, -2_1- | | |
| | | | GCATTGGGATACAGTGT | 0_bulge-asymmetric_A-, 0_1- | | |
| | | | | 1_mismatch_A-C, 4_1-1_mismatch_U- | | |
| | | | | C, 13_1-1_mismatch_A-G, 16 1- | | |
| | | | | 1_wobble_G-U, 32_6-6_internal_loop- | | |
| | | | | symmetric_AAAAAC-CCAAGC | | |
| 317 | 63A-63C | 871 (−16, +32) 0.98.53R2 | GGTGTGCCCTCTGATCCAAG | -16_6-6_internal_loop- | −16 | 32 |
| | | | CATCCCCATTCTACAGTAGG | symmetric_AUUGCA-GAGUCG, −7_1- | | |
| | | | ACTGAGCACTGCCGAGCTG | 1_wobble_U-G, -6_1-1_mismatch_G-G, | | |
| | | | GGCAATCTTGAGTCGGATGG | -4_0-1_bulge-asymmetric_-C, -2_1- | | |
| | | | CAGCATTGGGATACAGTGT | 0_bulge-asymmetric_A-, 0_1- | | |
| | | | | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 318 | 63A-63C | 871 (−16, +32) 0.100.55 | CTGGTGTGCCCTCTGATCCA | -16_6-6_internal_loop- | −16 | 32 |
| | | | AGCATCCCCATTCTACAGTA | symmetric_AUUGCA-GAGUCG, −7_1- | | |
| | | | GGACTGAGCACTGCCGAGC | 1_wobble_U-G, -6_1-1_mismatch_G-G, | | |
| | | | TGGGCAATCTTGAGTCGGAT | -4_0-1_bulge-asymmetric_-C, -2_1- | | |
| | | | GGCAGCATTGGGATACAGT | 0_bulge-asymmetric_A-, 0_1- | | |
| | | | GT | 1_mismatch_A-C, 4_1-1_mismatch_U- | | |
| | | | | C, 13_1-1_mismatch_A-G, 16_1- | | |
| | | | | 1_wobble_G-U, 32_6-6_internal_loop- | | |
| | | | | symmetric_AAAAAC-CCAAGC | | |
| 319 | 64A, 64P, | 919 0.113.57 (−22, +26) | CCCTGGTGTGCCCTCTGATG | −49_1-1_wobble_U-G, -22_6- | −22 | 25 |
| | 64Q, 64R | | TTTTTCAGTAGCTTCTACAG | 6_internal_loop-symmetric_GCCAUC- | | |
| | | | CAGTTCGGAGGAATCCCGA | AAGAGA, −3_1-1_wobble_U-G, -2_1- | | |
| | | | GGTCAGCAATCTTTGCAATA | 1_mismatch_A-A, 0_1-1_mismatch_A- | | |
| | | | AGAGAAGCATTGGGATACA | C, 2_1-1_mismatch_C-C, 6_1- | | |
| | | | GTGTGAAGAGCAGCA | 1_mismatch_G-G, 10_3−3_bulge- | | |
| | | | | symmetric_AGU-UCG, 25_7- | | |
| | | | | 7_internal_loop- | | |
| | | | | symmetric_UGGGGAU-CAGUAGC | | |
| 320 | 64A, 64L, | 919 0.113.57 (−20, +26) | CCCTGGTGTGCCCTCTGATG | −49_1-1_wobble_U-G, -20_6- | −20 | 25 |
| | 64M | | TTTTTTAGGGGCTTCTACAG | 6_internal_loop-symmetric_CAUCAU- | | |
| | | | CAGTTCGGAGGAATCCCGA | UACUAC, −3_1-1_wobble U-G, -2_1- | | |
| | | | GGTCAGCAATCTTTGCATAC | 1_mismatch_A-A, 0_1-1_mismatch_A- | | |
| | | | TACGCAGCATTGGGATACA | C, 2_1-1_mismatch_C-C, 6_1- | | |
| | | | GTGTGAAGAGCAGCA | 1_mismatch_G-G, 10_3−3_bulge- | | |
| | | | | symmetric_AGU-UCG, 25_7- | | |
| | | | | 7_internal_loop- | | |
| | | | | symmetric_UGGGGAU-UAGGGGC | | |
| 321 | 64A, 64S, | 919 0.113.57 (−18, +24) | CCCTGGTGTGCCCTCTGATG | −49_1-1_wobble_U-G, -18_6- | −18 | 24 |
| | 64T | | TTTTTATGGAACGTCTACAG | 6_internal_loop-symmetric_UCAUUG- | | |
| | | | CAGTTCGGAGGAATCCCGA | GUAGCU, −3_1-1_wobble_U-G, -2_1- | | |
| | | | GGTCAGCAATCTTTGGTAGC | 1_mismatch_A-A, 0_1-1_mismatch_A- | | |
| | | | TTGGCAGCATTGGGATACAG | C, 2_1-1_mismatch_C-C, 6_1- | | |
| | | | TGTGAAGAGCAGCA | 1_mismatch_G-G, 10_3−3_bulge- | | |
| | | | | symmetric_AGU-UCG, 24_6- | | |
| | | | | 6_internal_loop-symmetric_AUGGGG- | | |
| | | | | GGAACG | | |
| 322 | 64A, 64H, | 9190.113.57 (−18, +22) | CCCTGGTGTGCCCTCTGATG | −49_1-1_wobble_U-G, -18_6- | −18 | 22 |
| | 64I | | TTTTTATCCAGCGGGTACAG | 6_internal_loop-symmetric_UCAUUG- | | |
| | | | CAGTTCGGAGGAATCCCGA | GUCUUC, -3_1-1_wobble_U-G, -2_1- | | |
| | | | GGTCAGCAATCTTTGGTCTT | 1_mismatch_A-A, 0_1-1_mismatch_A- | | |
| | | | CTGGCAGCATTGGGATACA | C, 2_1-1_mismatch_C-C, 6_1- | | |
| | | | GTGTGAAGAGCAGCA | 1_mismatch_G-G, 10_3−3_bulge- | | |
| | | | | symmetric_AGU-UCG, 22_6- | | |
| | | | | 6_internal_loop-symmetric_GAAUGG- | | |
| | | | | AGCGGG | | |
| 323 | 64A, 64N, | 919 0.113.57 (−16, +28) | CCCTGGTGTGCCCTCTGATG | −49_1-1_wobble_U-G, -16_6- | −16 | N/A |
| | 640 | | TTTCAACGGCCCTTCTACAG | 6_internal_loop-symmetric_AUUGCA- | | |
| | | | CAGTTCGGAGGAATCCCGA | ACCCUG, −3_1-1_wobble_U-G, -2_1- | | |
| | | | GGTCAGCAATCTTACCCTGG | 1_mismatch_A-A, 0_1-1_mismatch_A- | | |
| | | | ATGGCAGCATTGGGATACA | C, 2_1-1_mismatch_C-C, 6_1- | | |
| | | | GTGTGAAGAGCAGCA | 1_mismatch_G-G, 10_3−3_bulge- | | |
| | | | | symmetric_AGU-UCG, 25_1-0_bulge- | | |
| | | | | asymmetric_U-, 29_5-6_internal_loop- | | |
| | | | | asymmetric_GAUAA-CAACGG | | |
| 324 | 64A, 64J, | 919 0.113.57 (−16, +24) | CCCTGGTGTGCCCTCTGATG | −49_1-1_wobble_U-G, -16_6- | −16 | 24 |
| | 64K | | TTTTTATTGAGCCTCTACAG | 6_internal_loop-symmetric_AUUGCA- | | |
| | | | CAGTTCGGAGGAATCCCGA | AAAUUA, −3_1-1_wobble_U-G, -2_1- | | |
| | | | GGTCAGCAATCTTAAATTAG | 1_mismatch_A-A, 0_1-1_mismatch_A- | | |
| | | | ATGGCAGCATTGGGATACA | C, 2_1-1_mismatch_C-C, 6_1- | | |
| | | | GTGTGAAGAGCAGCA | 1_mismatch_G-G, 10_3−3_bulge- | | |
| | | | | symmetric_AGU-UCG, 24_5- | | |
| | | | | 5_internal_loop-symmetric_AUGGG- | | |
| | | | | GAGCC, 29_1-1_wobble_G-U | | |
| 325 | 64A, 64F, | 919 0.113.57 (−14, +26) | CCCTGGTGTGCCCTCTGATG | −49_1-1_wobble_U-G, −14_6- | −14 | 25 |
| | 64G | | TTTTTTAGGGGCTTCTACAG | 6_internal_loop-symmetric_UGCAAA- | | |
| | | | CAGTTCGGAGGAATCCCGA | AAACGU, −3_1-1_wobble_U-G, -2_1- | | |
| | | | GGTCAGCAATCAAACGTAT | 1_mismatch_A-A, 0_1-1_mismatch_A- | | |
| | | | GATGGCAGCATTGGGATAC | C, 2_1-1_mismatch_C-C, 6_1- | | |
| | | | AGTGTGAAGAGCAGCA | 1_mismatch_G-G, 10_3−3_bulge- | | |
| | | | | symmetric_AGU-UCG, 25_7- | | |
| | | | | 7_internal_loop- | | |
| | | | | symmetric_UGGGGAU-UAGGGGC | | |
| 50 | 64A-64C | 919 0.113.57 (−12, +24) | CCCTGGTGTGCCCTCTGATG | −49_1-1_wobble_U-G, -12_6- | −12 | 24 |
| | | | TTTTTATGAGGCGTCTACAG | 6_internal_loop-symmetric_CAAAGA- | | |
| | | | CAGTTCGGAGGAATCCCGA | GACUAA, −3_1-1_wobble_U-G, -2_1- | | |
| | | | GGTCAGCAAGACTAACAAT | 1_mismatch_A-A, 0_1-1_mismatch_A- | | |
| | | | GATGGCAGCATTGGGATAC | C, 2_1-1_mismatch_C-C, 6_1- | | |
| | | | AGTGTGAAGAGCAGCA | 1_mismatch_G-G, 10_3−3_bulge- | | |
| | | | | symmetric_AGU-UCG, 24_6- | | |
| | | | | 6_internal_loop-symmetric_AUGGGG- | | |
| | | | | GAGGCG | | |
| 326 | 64A, 64D, | 919 0.113.57 (−10, +24) | CCCTGGTGTGCCCTCTGATG | −49_1-1_wobble_U-G, -10_6- | −10 | 24 |
| | 64E | | TTTTTATTGAGCCTCTACAG | 6_internal_loop-symmetric_AAGAUU- | | |
| | | | CAGTTCGGAGGAATCCCGA | UUAGCC, −3_1-1_wobble_U-G, -2_1- | | |
| | | | GGTCAGCTTAGCCTGCAATG | 1_mismatch_A-A, 0_1-1_mismatch_A- | | |
| | | | ATGGCAGCATTGGGATACA | C, 2_1-1_mismatch_C-C, 6_1- | | |
| | | | GTGTGAAGAGCAGCA | 1_mismatch_G-G, 10_3−3_bulge- | | |
| | | | | symmetric_AGU-UCG, 24_5- | | |
| | | | | 5_internal_loop-symmetric_AUGGG- | | |
| | | | | GAGCC, 29_1-1_wobble_G-U | | |
| 327 | 65A-65C | 919 (−12, +24) 0.100.75 | AACTTCAGGTGCACGAAAC | -12_6-6_internal_loop- | −12 | 24 |
| | | | CCTGGTGTGCCCTCTGATGT | symmetric_CAAAGA-GACUAA, -3_1- | | |
| | | | TTTTATGAGGCGTCTACAGC | 1_wobble_U-G, -2_1-1_mismatch_A-A, | | |
| | | | AGTTCGGAGGAATCCCGAG | 0_1-1_mismatch_A-C, 2_1- | | |
| | | | GTCAGCAAGACTAACAATG | 1_mismatch_C-C, 6_1-1_mismatch_G- | | |
| | | | ATG | G, 10_3−3_bulge-symmetric_AGU- | | |
| | | | | UCG, 24_6-6_internal_loop- | | |
| | | | | symmetric_AUGGGG-GAGGCG | | |
| 328 | 65A-65C | 919 (−12, +24) 0.100.70 | CAGGTGCACGAAACCCTGG | -12_6-6_internal_loop- | −12 | 24 |
| | | | TGTGCCCTCTGATGTTTTTAT | symmetric_CAAAGA-GACUAA, -3_1- | | |
| | | | GAGGCGTCTACAGCAGTTCG | 1_wobble_U-G, -2_1-1_mismatch_A-A, | | |
| | | | GAGGAATCCCGAGGTCAGC | 0_1-1_mismatch_A-C, 2_1- | | |
| | | | AAGACTAACAATGATGGCA | 1_mismatch_C-C, 6_1-1_mismatch_G- | | |
| | | | GC | G, 10_3−3_bulge-symmetric_AGU- | | |
| | | | | UCG, 24_6-6_internal_loop- | | |
| | | | | symmetric_AUGGGG-GAGGCG | | |
| 329 | 65A-65C | 919 (−12, +24) 0.100.65 | GCACGAAACCCTGGTGTGCC | -12_6-6_internal_loop- | −12 | 24 |
| | | | CTCTGATGTTTTTATGAGGC | symmetric_CAAAGA-GACUAA, −3_1- | | |
| | | | GTCTACAGCAGTTCGGAGG | 1_wobble_U-G, -2_1-1_mismatch_A-A, | | |
| | | | AATCCCGAGGTCAGCAAGA | 0_1-1_mismatch_A-C, 2_1- | | |
| | | | CTAACAATGATGGCAGCATT | 1_mismatch_C-C, 6_1-1_mismatch_G- | | |
| | | | GG | G, 10_3−3_bulge-symmetric_AGU- | | |
| | | | | UCG, 24_6-6_internal_loop- | | |
| | | | | symmetric_AUGGGG-GAGGCG | | |
| 330 | 65A-65C | 919 (−12, +24) 0.100.60 | AAACCCTGGTGTGCCCTCTG | -12_6-6_internal_loop- | −12 | 24 |
| | | | ATGTTTTTATGAGGCGTCTA | symmetric_CAAAGA-GACUAA, −3_1- | | |
| | | | CAGCAGTTCGGAGGAATCC | 1_wobble_U-G, -2_1-1_mismatch_A-A, | | |
| | | | CGAGGTCAGCAAGACTAAC | 0_1-1_mismatch_A-C, 2_1- | | |
| | | | AATGATGGCAGCATTGGGA | 1_mismatch_C-C, 6_1-1_mismatch_G- | | |
| | | | TAC | G, 10_3−3_bulge-symmetric_AGU- | | |
| | | | | UCG, 24_6-6_internal_loop- | | |
| | | | | symmetric_AUGGGG-GAGGCG | | |
| 331 | 65A-65C | 919 (−12, +24) 0.100.55 | CTGGTGTGCCCTCTGATGTT | -12_6-6_internal_loop- | −12 | 24 |
| | | | TTTATGAGGCGTCTACAGCA | symmetric_CAAAGA-GACUAA, −3_1- | | |
| | | | GTTCGGAGGAATCCCGAGG | 1_wobble_U-G, -2_1-1_mismatch_A-A, | | |
| | | | TCAGCAAGACTAACAATGA | 0_1-1_mismatch_A-C, 2_1- | | |
| | | | TGGCAGCATTGGGATACAGT | 1_mismatch_C-C, 6_1-1_mismatch_G- | | |
| | | | GT | G, 10_3−3_bulge-symmetric_AGU- | | |
| | | | | UCG, 24_6-6_internal_loop- | | |
| | | | | symmetric_AUGGGG-GAGGCG | | |
| 332 | 65A-65C | 919 (−12, +24) 0.100.50 | GTGCCCTCTGATGTTTTTAT | -12_6-6_internal_loop- | −12 | 24 |
| | | | GAGGCGTCTACAGCAGTTCG | symmetric_CAAAGA-GACUAA, −3_1- | | |
| | | | GAGGAATCCCGAGGTCAGC | 1_wobble_U-G, -2_1-1_mismatch_A-A, | | |
| | | | AAGACTAACAATGATGGCA | 0_1-1_mismatch_A-C, 2_1- | | |
| | | | GCATTGGGATACAGTGTGA | 1_mismatch_C-C, 6_1-1_mismatch_G- | | |
| | | | AAA | G, 10_3−3_bulge-symmetric_AGU- | | |
| | | | | UCG, 24_6-6_internal_loop- | | |
| | | | | symmetric_AUGGGG-GAGGCG | | |
| 50 | 65A-65C | 919 (−12, +24) 0.113.57 | CCCTGGTGTGCCCTCTGATG | −49_1-1_wobble_U-G, -12_6- | −12 | 24 |
| | | | TTTTTATGAGGCGTCTACAG | 6_internal_loop-symmetric_CAAAGA- | | |
| | | | CAGTTCGGAGGAATCCCGA | GACUAA, −3_1-1_wobble_U-G, -2_1- | | |
| | | | GGTCAGCAAGACTAACAAT | 1_mismatch_A-A, 0_1-1_mismatch_A- | | |
| | | | GATGGCAGCATTGGGATAC | C, 2_1-1_mismatch_C-C, 6_1- | | |
| | | | AGTGTGAAGAGCAGCA | 1_mismatch_G-G, 10_3−3_bulge- | | |
| | | | | symmetric_AGU-UCG, 24_6- | | |
| | | | | 6_internal_loop-symmetric_AUGGGG- | | |
| | | | | GAGGCG | | |
| 333 | 66A | 919 (−12, +34) 0.100.50 | GTGCCCTCTGTACAAATTAT | -12_6-6_internal_loop- | −12 | 34 |
| | | | CCCCATTCTACAGCAGTTCG | symmetric_CAAAGA-GACUAA, -3_1- | | |
| | | | GAGGAATCCCGAGGTCAGC | 1_wobble_U-G, -2_1-1_mismatch_A-A, | | |
| | | | AAGACTAACAATGATGGCA | 0_1-1_mismatch_A-C, 2_1- | | |
| | | | GCATTGGGATACAGTGTGA | 1_mismatch_C-C, 6_1-1_mismatch_G- | | |
| | | | AAA | G, 10_3−3_bulge-symmetric_AGU- | | |
| | | | | UCG, 34_6-6_internal_loop- | | |
| | | | | symmetric_AAACAU-UACAAA | | |
| 334 | 66A | 919 (−12, +32) 0.100.50 | GTGCCCTCTGATCAAAGCAT | -12_6-6_internal_loop- | −12 | 32 |
| | | | CCCCATTCTACAGCAGTTCG | symmetric_CAAAGA-GACUAA, -3_1- | | |
| | | | GAGGAATCCCGAGGTCAGC | 1_wobble_U-G, -2_1-1_mismatch_A-A, | | |
| | | | AAGACTAACAATGATGGCA | 0_1-1_mismatch_A-C, 2_1- | | |
| | | | GCATTGGGATACAGTGTGA | 1_mismatch_C-C, 6_1-1_mismatch_G- | | |
| | | | AAA | G, 10_3−3_bulge-symmetric_AGU- | | |
| | | | | UCG, 32_6-6_internal_loop- | | |
| | | | | symmetric_AAAAAC-CAAAGC | | |
| 335 | 66A | 919 (−12, +30) 0.100.50 | GTGCCCTCTGATGTAAGCTA | -12_6-6_internal_loop- | −12 | 30 |
| | | | CCCCATTCTACAGCAGTTCG | symmetric_CAAAGA-GACUAA, -3_1- | | |
| | | | GAGGAATCCCGAGGTCAGC | 1_wobble_U-G, -2_1-1_mismatch_A-A, | | |
| | | | AAGACTAACAATGATGGCA | 0_1-1_mismatch_A-C, 2_1- | | |
| | | | GCATTGGGATACAGTGTGA | 1_mismatch_C-C, 6_1-1_mismatch_G- | | |
| | | | AAA | G, 10_3−3_bulge-symmetric_AGU- | | |
| | | | | UCG, 30_6-6_internal_loop- | | |
| | | | | symmetric_AUAAAA-AAGCUA | | |
| 336 | 66A | 919 (−12, +28) 0.100.50 | GTGCCCTCTGATGTTTGCTA | -12_6-6_internal_loop- | −12 | 28 |
| | | | GACCATTCTACAGCAGTTCG | symmetric_CAAAGA-GACUAA, -3_1- | | |
| | | | GAGGAATCCCGAGGTCAGC | 1_wobble_U-G, -2_1-1_mismatch_A-A, | | |
| | | | AAGACTAACAATGATGGCA | 0_1-1_mismatch_A-C, 2_1- | | |
| | | | GCATTGGGATACAGTGTGA | 1_mismatch_C-C, 6_1-1_mismatch_G- | | |
| | | | AAA | G, 10_3−3_bulge-symmetric_AGU- | | |
| | | | | UCG, 28_6-6_internal_loop- | | |
| | | | | symmetric_GGAUAA-GCUAGA | | |
| 337 | 66A | 919 (−12, +26) 0.100.50 | GTGCCCTCTGATGTTTTTTA | -12_6-6_internal_loop- | −12 | 26 |
| | | | GAGGATTCTACAGCAGTTCG | symmetric_CAAAGA-GACUAA, −3_1- | | |
| | | | GAGGAATCCCGAGGTCAGC | 1_wobble_U-G, -2_1-1_mismatch_A-A, | | |
| | | | AAGACTAACAATGATGGCA | 0_1-1_mismatch_A-C, 2_1- | | |
| | | | GCATTGGGATACAGTGTGA | 1_mismatch_C-C, 6_1-1_mismatch_G- | | |
| | | | AAA | G, 10_3−3_bulge-symmetric_AGU- | | |
| | | | | UCG, 26_6-6_internal_loop- | | |
| | | | | symmetric_GGGGAU-UAGAGG | | |
| 338 | 66A-66C | 919 (−12, +24) 0.100.50 | GTGCCCTCTGATGTTTTTAT | -12_6-6_internal_loop- | −12 | 24 |
| | | | GAGGCGTCTACAGCAGTTCG | symmetric_CAAAGA-GACUAA, −3_1- | | |
| | | | GAGGAATCCCGAGGTCAGC | 1_wobble_U-G, -2_1-1_mismatch_A-A, | | |
| | | | AAGACTAACAATGATGGCA | 0_1-1_mismatch_A-C, 2_1- | | |
| | | | GCATTGGGATACAGTGTGA | 1_mismatch_C-C, 6_1-1_mismatch_G- | | |
| | | | AAA | G, 10_3−3_bulge-symmetric_AGU- | | |
| | | | | UCG, 24_6-6_internal_loop- | | |
| | | | | symmetric_AUGGGG-GAGGCG | | |
| 339 | 66A-66C | 919 (−12, +22) 0.100.50 | GTGCCCTCTGATGTTTTTAT | -12_6-6_internal_loop- | −12 | 22 |
| | | | CCGGCGAGTACAGCAGTTC | symmetric_CAAAGA-GACUAA, −3_1- | | |
| | | | GGAGGAATCCCGAGGTCAG | 1_wobble_U-G, -2_1-1_mismatch_A-A, | | |
| | | | CAAGACTAACAATGATGGC | 0_1-1_mismatch_A-C, 2_1- | | |
| | | | AGCATTGGGATACAGTGTG | 1_mismatch_C-C, 6_1-1_mismatch_G- | | |
| | | | AAAA | G, 10_3−3_bulge-symmetric_AGU- | | |
| | | | | UCG, 22_6-6_internal_loop- | | |
| | | | | symmetric_GAAUGG-GGCGAG | | |
| 340 | 66A | 919 (−12, +20) 0.100.50 | GTGCCCTCTGATGTTTTTAT | -12_6-6_internal_loop- | −12 | 20 |
| | | | CCCCCGAGATCAGCAGTTCG | symmetric_CAAAGA-GACUAA, -3_1- | | |
| | | | GAGGAATCCCGAGGTCAGC | 1_wobble_U-G, -2_1-1_mismatch_A-A, | | |
| | | | AAGACTAACAATGATGGCA | 0_1-1_mismatch_A-C, 2_1- | | |
| | | | GCATTGGGATACAGTGTGA | 1_mismatch_C-C, 6_1-1_mismatch_G- | | |
| | | | AAA | G, 10_3−3_bulge-symmetric_AGU- | | |
| | | | | UCG, 20_6-6_internal_loop- | | |
| | | | | symmetric_UAGAAU-CGAGAU | | |
| 341 | 66A | 919 (−12, +18) 0.100.50 | GTGCCCTCTGATGTTTTTAT | -12_6-6_internal_loop- | −12 | 18 |
| | | | CCCCATAGATGTGCAGTTCG | symmetric_CAAAGA-GACUAA, −3_1- | | |
| | | | GAGGAATCCCGAGGTCAGC | 1_wobble_U-G, -2_1-1_mismatch_A-A, | | |
| | | | AAGACTAACAATGATGGCA | 0_1-1_mismatch_A-C, 2_1- | | |
| | | | GCATTGGGATACAGTGTGA | 1_mismatch_C-C, 6_1-1_mismatch_G- | | |
| | | | AAA | G, 10_3−3_bulge-symmetric_AGU- | | |
| | | | | UCG, 18_6-6_internal_loop- | | |
| | | | | symmetric_UGUAGA-AGAUGU | | |
| 342 | 67A-67C | 919 (−24, +22) 0.100.50 | GTGCCCTCTGATGTTTTTAT | -24_6-6_internal_loop- | −24 | 22 |
| | | | CCGGCGAGTACAGCAGTTC | symmetric_CUGCCA-ACCGUC, −3_1- | | |
| | | | GGAGGAATCCCGAGGTCAG | 1_wobble_U-G, -2_1-1_mismatch_A-A, | | |
| | | | CAATCTTTGCAATGAACCGT | 0_1-1_mismatch_A-C, 2_1- | | |
| | | | CCATTGGGATACAGTGTGAA | 1_mismatch_C-C, 6_1-1_mismatch_G- | | |
| | | | AA | G, 10_3−3_bulge-symmetric_AGU- | | |
| | | | | UCG, 22_6-6_internal_loop- | | |
| | | | | symmetric_GAAUGG-GGCGAG | | |
| 343 | 67A-67C | 919 (−22, +22) 0.100.50 | GTGCCCTCTGATGTTTTTAT | -22_6-6_internal_loop- | −22 | 22 |
| | | | CCGGCGAGTACAGCAGTTC | symmetric_GCCAUC-CUACCG, -3_1- | | |
| | | | GGAGGAATCCCGAGGTCAG | 1_wobble_U-G, -2_1-1_mismatch_A-A, | | |
| | | | CAATCTTTGCAATCTACCGA | 0_1-1_mismatch_A-C, 2_1- | | |
| | | | GCATTGGGATACAGTGTGA | 1_mismatch_C-C, 6 1-1_mismatch_G- | | |
| | | | AAA | G, 10_3−3_bulge-symmetric_AGU- | | |
| | | | | UCG, 22_6-6_internal_loop- | | |
| | | | | symmetric_GAAUGG-GGCGAG | | |
| 344 | 67A-67C | 919 (−20, +22) 0.100.50 | GTGCCCTCTGATGTTTTTAT | -20_6-6_internal_loop- | −20 | 22 |
| | | | CCGGCGAGTACAGCAGTTC | symmetric_CAUCAU-UACUAC, −3_1- | | |
| | | | GGAGGAATCCCGAGGTCAG | 1_wobble_U-G, -2_1-1_mismatch_A-A, | | |
| | | | CAATCTTTGCATACTACGCA | 0_1-1_mismatch_A-C, 2_1- | | |
| | | | GCATTGGGATACAGTGTGA | 1_mismatch_C-C, 6_1-1_mismatch_G- | | |
| | | | AAA | G, 10_3−3_bulge-symmetric_AGU- | | |
| | | | | UCG, 22_6-6_internal_loop- | | |
| | | | | symmetric_GAAUGG-GGCGAG | | |
| 345 | 67A-67C | 919 (−18, +22) 0.100.50 | GTGCCCTCTGATGTTTTTAT | -18_6-6_internal_loop- | −18 | 22 |
| | | | CCGGCGAGTACAGCAGTTC | symmetric_UCAUUG-GUUACU, −3_1- | | |
| | | | GGAGGAATCCCGAGGTCAG | 1_wobble_U-G, -2_1-1_mismatch_A-A, | | |
| | | | CAATCTTTGGTTACTTGGCA | 0_1-1_mismatch_A-C, 2_1- | | |
| | | | GCATTGGGATACAGTGTGA | 1_mismatch_C-C, 6_1-1_mismatch_G- | | |
| | | | AAA | G, 10_3−3_bulge-symmetric_AGU- | | |
| | | | | UCG, 22_6-6_internal_loop- | | |
| | | | | symmetric_GAAUGG-GGCGAG | | |
| 346 | 67A-67C | 919 (−16, +22) 0.100.50 | GTGCCCTCTGATGTTTTTAT | -16_6-6_internal_loop- | −16 | 22 |
| | | | CCGGCGAGTACAGCAGTTC | symmetric_AUUGCA-AAGUUA, −3_1- | | |
| | | | GGAGGAATCCCGAGGTCAG | 1_wobble_U-G, -2_1-1_mismatch_A-A, | | |
| | | | CAATCTTAAGTTAGATGGCA | 0_1-1_mismatch_A-C, 2_1- | | |
| | | | GCATTGGGATACAGTGTGA | 1_mismatch_C-C, 6_1-1_mismatch_G- | | |
| | | | AAA | G, 10_3−3_bulge-symmetric_AGU- | | |
| | | | | UCG, 22_6-6_internal_loop- | | |
| | | | | symmetric_GAAUGG-GGCGAG | | |
| 347 | 67A-67C | 919 (−14, +22) 0.100.50 | GTGCCCTCTGATGTTTTTAT | −14_6-6_internal_loop- | −14 | 22 |
| | | | CCGGCGAGTACAGCAGTTC | symmetric_UGCAAA-CUAAGU, −3_1- | | |
| | | | GGAGGAATCCCGAGGTCAG | 1_wobble_U-G, -2_1-1_mismatch_A-A, | | |
| | | | CAATCCTAAGTATGATGGCA | 0_1-1_mismatch_A-C, 2_1- | | |
| | | | GCATTGGGATACAGTGTGA | 1_mismatch_C-C, 6_1-1_mismatch_G- | | |
| | | | AAA | G, 10_3−3_bulge-symmetric_AGU- | | |
| | | | | UCG, 22_6-6_internal_loop- | | |
| | | | | symmetric_GAAUGG-GGCGAG | | |
| 348 | 67A-67C | 919 (−12, +22) 0.100.50 | GTGCCCTCTGATGTTTTTAT | -12 6-6_internal_loop- | −12 | 22 |
| | | | CCGGCGAGTACAGCAGTTC | symmetric_CAAAGA-GACUAA, −3_1- | | |
| | | | GGAGGAATCCCGAGGTCAG | 1_wobble_U-G, -2_1-1_mismatch_A-A, | | |
| | | | CAAGACTAACAATGATGGC | 0_1-1_mismatch_A-C, 2_1- | | |
| | | | AGCATTGGGATACAGTGTG | 1_mismatch_C-C, 6_1-1_mismatch_G- | | |
| | | | AAAA | G, 10_3−3_bulge-symmetric_AGU- | | |
| | | | | UCG, 22_6-6_internal_loop- | | |
| | | | | symmetric_GAAUGG-GGCGAG | | |
| 349 | 67A-67C | 919 (−9, +22) 0.100.50 | GTGCCCTCTGATGTTTTTAT | -9_6-6_internal_loop- | −9 | 22 |
| | | | CCGGCGAGTACAGCAGTTC | symmetric_AGAUUG-GUUGAC, −3_1- | | |
| | | | GGAGGAATCCCGAGGTCAG | 1_wobble_U-G, -2_1-1_mismatch_A-A, | | |
| | | | GTTGACTTGCAATGATGGCA | 0_1-1_mismatch_A-C, 2_1- | | |
| | | | GCATTGGGATACAGTGTGA | 1_mismatch_C-C, 6_1-1_mismatch_G- | | |
| | | | AAA | G, 10_3−3_bulge-symmetric_AGU- | | |
| | | | | UCG, 22_6-6_internal_loop- | | |
| | | | | symmetric_GAAUGG-GGCGAG | | |
| 350 | 67A-67C | 919 (−7, +22) 0.100.50 | GTGCCCTCTGATGTTTTTAT | −7_6-6_internal_loop- | −7 | 22 |
| | | | CCGGCGAGTACAGCAGTTC | symmetric_AUUGCU-UCGUUG, −3_1- | | |
| | | | GGAGGAATCCCGAGGTCTC | 1_wobble_U-G, -2_1-1_mismatch_A-A, | | |
| | | | GTTGCTTTGCAATGATGGCA | 0_1-1_mismatch_A-C, 2_1- | | |
| | | | GCATTGGGATACAGTGTGA | 1_mismatch_C-C, 6_1-1_mismatch_G- | | |
| | | | AAA | G, 10_3−3_bulge-symmetric_AGU- | | |
| | | | | UCG, 22_6-6_internal_loop- | | |
| | | | | symmetric_GAAUGG-GGCGAG | | |
| 351 | 68A-68C | 919 (−14, +22) | GTGCCCTCTGATGTTTTTAT | −14_6-6_internal_loop- | −14 | 22 |
| | | 0.90.50L 10 | CCGGCGAGTACAGCAGTTC | symmetric_UGCAAA-CUAAGU, −3_1- | | |
| | | | GGAGGAATCCCGAGGTCAG | 1_wobble_U-G, -2_1-1_mismatch_A-A, | | |
| | | | CAATCCTAAGTATGATGGCA | 0_1-1_mismatch_A-C, 2_1- | | |
| | | | GCATTGGGATAC | 1_mismatch_C-C, 6_1-1_mismatch_G- | | |
| | | | | G, 10_3−3_bulge-symmetric_AGU- | | |
| | | | | UCG, 22_6-6_internal_loop- | | |
| | | | | symmetric_GAAUGG-GGCGAG | | |
| 352 | 68A-68C | 919 (−14, +22) 0.92.50L8 | GTGCCCTCTGATGTTTTTAT | −14_6-6_internal_loop- | −14 | 22 |
| | | | CCGGCGAGTACAGCAGTTC | symmetric_UGCAAA-CUAAGU, −3_1- | | |
| | | | GGAGGAATCCCGAGGTCAG | 1_wobble_U-G, -2_1-1_mismatch_A-A, | | |
| | | | CAATCCTAAGTATGATGGCA | 0_1-1_mismatch_A-C, 2_1- | | |
| | | | GCATTGGGATACAG | 1_mismatch_C-C, 6_1-1_mismatch_G- | | |
| | | | | G, 10_3−3_bulge-symmetric_AGU- | | |
| | | | | UCG, 22_6-6_internal_loop- | | |
| | | | | symmetric_GAAUGG-GGCGAG | | |
| 353 | 68A-68C | 919 (−14, +22) 0.94.50L6 | GTGCCCTCTGATGTTTTTAT | −14_6-6_internal_loop- | −14 | 22 |
| | | | CCGGCGAGTACAGCAGTTC | symmetric_UGCAAA-CUAAGU, -3_1- | | |
| | | | GGAGGAATCCCGAGGTCAG | 1_wobble_U-G, -2_1-1_mismatch_A-A, | | |
| | | | CAATCCTAAGTATGATGGCA | 0_1-1_mismatch_A-C, 2_1- | | |
| | | | GCATTGGGATACAGTG | 1_mismatch_C-C, 6 1-1_mismatch_G- | | |
| | | | | G, 10_3−3_bulge-symmetric_AGU- | | |
| | | | | UCG, 22_6-6_internal_loop- | | |
| | | | | symmetric_GAAUGG-GGCGAG | | |
| 354 | 68A-68C | 919 (−14, +22) 0.96.50L4 | GTGCCCTCTGATGTTTTTAT | −14_6-6_internal_loop- | −14 | 22 |
| | | | CCGGCGAGTACAGCAGTTC | symmetric_UGCAAA-CUAAGU, −3_1- | | |
| | | | GGAGGAATCCCGAGGTCAG | 1_wobble_U-G, -2_1-1_mismatch_A-A, | | |
| | | | CAATCCTAAGTATGATGGCA | 0 1-1_mismatch_A-C, 2_1- | | |
| | | | GCATTGGGATACAGTGTG | 1_mismatch_C-C, 6_1-1_mismatch_G- | | |
| | | | | G, 10_3−3_bulge-symmetric_AGU- | | |
| | | | | UCG, 22_6-6_internal_loop- | | |
| | | | | symmetric_GAAUGG-GGCGAG | | |
| 355 | 68A-68C | 919 (−14, +22) 0.98.50L2 | GTGCCCTCTGATGTTTTTAT | −14_6-6_internal_loop- | −14 | 22 |
| | | | CCGGCGAGTACAGCAGTTC | symmetric_UGCAAA-CUAAGU, −3_1- | | |
| | | | GGAGGAATCCCGAGGTCAG | 1_wobble_U-G, -2_1-1_mismatch_A-A, | | |
| | | | CAATCCTAAGTATGATGGCA | 0_1-1_mismatch_A-C, 2_1- | | |
| | | | GCATTGGGATACAGTGTGA | 1_mismatch_C-C, 6_1-1_mismatch_G- | | |
| | | | A | G, 10_3−3_bulge-symmetric_AGU- | | |
| | | | | UCG, 22_6-6_internal_loop- | | |
| | | | | symmetric_GAAUGG-GGCGAG | | |
| 356 | 68A-68C | 919 (−14, +22) | ATGTTTTTATCCGGCGAGTA | −14_6-6_internal_loop- | −14 | 22 |
| | | 0.90.40R10 | CAGCAGTTCGGAGGAATCC | symmetric_UGCAAA-CUAAGU, −3_1- | | |
| | | | CGAGGTCAGCAATCCTAAGT | 1_wobble_U-G, -2_1-1_mismatch_A-A, | | |
| | | | ATGATGGCAGCATTGGGAT | 0_1-1_mismatch_A-C, 2_1- | | |
| | | | ACAGTGTGAAAA | 1_mismatch_C-C, 6_1-1_mismatch_G- | | |
| | | | | G, 10_3−3_bulge-symmetric_AGU- | | |
| | | | | UCG, 22_6-6_internal_loop- | | |
| | | | | symmetric_GAAUGG-GGCGAG | | |
| 357 | 68A-68C | 919 (−14, +22) 0.92.42R8 | TGATGTTTTTATCCGGCGAG | −14_6-6_internal_loop- | −14 | 22 |
| | | | TACAGCAGTTCGGAGGAAT | symmetric_UGCAAA-CUAAGU, −3_1- | | |
| | | | CCCGAGGTCAGCAATCCTAA | 1_wobble_U-G, -2_1-1_mismatch_A-A, | | |
| | | | GTATGATGGCAGCATTGGG | 0_1-1_mismatch_A-C, 2_1- | | |
| | | | ATACAGTGTGAAAA | 1_mismatch_C-C, 6 1-1_mismatch_G- | | |
| | | | | G, 10_3−3_bulge-symmetric_AGU- | | |
| | | | | UCG, 22_6-6_internal_loop- | | |
| | | | | symmetric_GAAUGG-GGCGAG | | |
| 358 | 68A-68C | 919 (−14, +22) 0.94.44R6 | TCTGATGTTTTTATCCGGCG | −14_6-6_internal_loop- | −14 | 22 |
| | | | AGTACAGCAGTTCGGAGGA | symmetric_UGCAAA-CUAAGU, −3_1- | | |
| | | | ATCCCGAGGTCAGCAATCCT | 1_wobble_U-G, -2_1-1_mismatch_A-A, | | |
| | | | AAGTATGATGGCAGCATTG | 0_1-1_mismatch_A-C, 2_1- | | |
| | | | GGATACAGTGTGAAAA | 1_mismatch_C-C, 6_1-1_mismatch_G- | | |
| | | | | G, 10_3−3_bulge-symmetric_AGU- | | |
| | | | | UCG, 22_6-6_internal_loop- | | |
| | | | | symmetric_GAAUGG-GGCGAG | | |
| 359 | 68A-68C | 919 (−14, +22) 0.96.46R4 | CCTCTGATGTTTTTATCCGG | −14_6-6_internal_loop- | −14 | 22 |
| | | | CGAGTACAGCAGTTCGGAG | symmetric_UGCAAA-CUAAGU, −3_1- | | |
| | | | GAATCCCGAGGTCAGCAAT | 1_wobble_U-G, -2_1-1_mismatch_A-A, | | |
| | | | CCTAAGTATGATGGCAGCAT | 0_1-1_mismatch_A-C, 2_1- | | |
| | | | TGGGATACAGTGTGAAAA | 1_mismatch_C-C, 6_1-1_mismatch_G- | | |
| | | | | G, 10_3−3_bulge-symmetric_AGU- | | |
| | | | | UCG, 22_6-6_internal_loop- | | |
| | | | | symmetric_GAAUGG-GGCGAG | | |
| 360 | 68A-68C | 919 (−14, +22) 0.98.48R2 | GCCCTCTGATGTTTTTATCC | −14_6-6_internal_loop- | −14 | 22 |
| | | | GGCGAGTACAGCAGTTCGG | symmetric_UGCAAA-CUAAGU, −3_1- | | |
| | | | AGGAATCCCGAGGTCAGCA | 1_wobble_U-G, -2_1-1_mismatch_A-A, | | |
| | | | ATCCTAAGTATGATGGCAGC | 0_1-1_mismatch_A-C, 2_1- | | |
| | | | ATTGGGATACAGTGTGAAA | 1_mismatch_C-C, 6_1-1_mismatch_G- | | |
| | | | A | G, 10_3−3_bulge-symmetric_AGU- | | |
| | | | | UCG, 22_6-6_internal_loop- | | |
| | | | | symmetric_GAAUGG-GGCGAG | | |
| 361 | 68A-68C | 919 (−14, +22) 0.100.50 | GTGCCCTCTGATGTTTTTAT | −14_6-6_internal_loop- | −14 | 22 |
| | | | CCGGCGAGTACAGCAGTTC | symmetric_UGCAAA-CUAAGU, −3_1- | | |
| | | | GGAGGAATCCCGAGGTCAG | 1_wobble_U-G, -2_1-1_mismatch_A-A, | | |
| | | | CAATCCTAAGTATGATGGCA | 0_1-1_mismatch_A-C, 2_1- | | |
| | | | GCATTGGGATACAGTGTGA | 1_mismatch_C-C, 6_1-1_mismatch_G- | | |
| | | | AAA | G, 10_3−3_bulge-symmetric_AGU- | | |
| | | | | UCG, 22_6-6_internal_loop- | | |
| | | | | symmetric_GAAUGG-GGCGAG | | |
| 362 | 69A | 844 (−12, +18) 0.113.57 | CCCTGGTGTGCCCTCTGATG | −49_1-1_wobble_U-G, -12_8- | −12 | 18 |
| | | | TTTTTATCCCCATCACCCTG | 8_internal_loop- | | |
| | | | CAGTTCATAGCAATCCCGTA | symmetric_UGCAAAGA- | | |
| | | | GTCAACAACAATATCCATGA | CAAUAUCC, -8_1-1_mismatch_C-A, | | |
| | | | TGGCAGCATTGGGATACAGT | 0_1-1_mismatch_A-C, 2_1- | | |
| | | | GTGAAGAGCAGCA | 1_mismatch_C-C, 9_2-2_bulge- | | |
| | | | | symmetric_CA-AU, 12_1- | | |
| | | | | 1_mismatch_U-U, 18_6- | | |
| | | | | 6_internal_loop-symmetric_UGUAGA- | | |
| | | | | CACCCU | | |
| 363 | 69B | 844 (−12, +26) 0.113.57 | CCCTGGTGTGCCCTCTGATG | -49 1-1_wobble_U-G, -12_8- | −12 | 26 |
| | | | TTTTTCGGAGGATTCTACGG | 8_internal_loop- | | |
| | | | CAGTTCATAGCAATCCCGTA | symmetric_UGCAAAGA- | | |
| | | | GTCAACAAGGGTCCCCATG | GGGUCCCC, -8_1-1_mismatch_C-A, | | |
| | | | ATGGCAGCATTGGGATACA | 0_1-1_mismatch_A-C, 2_1- | | |
| | | | GTGTGAAGAGCAGCA | 1_mismatch_C-C, 9_2-2_bulge- | | |
| | | | | symmetric_CA-AU, 12_1- | | |
| | | | | 1_mismatch_U-U, 18_1-1_wobble_U-G, | | |
| | | | | 26_6-6_internal_loop- | | |
| | | | | symmetric_GGGGAU-CGGAGG | | |
| 364 | 69C | 844 (−22, +26) 0.113.57 | CCCTGGTGTGCCCTCTGATG | −49_1-1_wobble_U-G, -19_9- | −19 | 27 |
| | | | TTTTTCTAGATATTCTACGG | 9_internal_loop- | | |
| | | | CAGTTCATAGCAATCCCGTA | symmetric_GCCAUCAUU- | | |
| | | | GTCAACAATCTTTGCCATTA | CAUUAUUUG, -8_1-1_mismatch_C- | | |
| | | | TTTGAGCATTGGGATACAGT | A, 0_1-1_mismatch_A-C, 2_1- | | |
| | | | GTGAAGAGCAGCA | 1_mismatch_C-C, 9_2-2_bulge- | | |
| | | | | symmetric_CA-AU, 12_1- | | |
| | | | | 1_mismatch_U-U, 18_1-1_wobble_U-G, | | |
| | | | | 26_1-1_wobble_G-U, 27_5- | | |
| | | | | 5_internal_loop-symmetric_GGGAU- | | |
| | | | | CUAGA | | |
| 365 | 70A | 1976 (−16, +16) 0.113.57 | CCCTGGTGTGCCCTCTGATG | −49_1-1_wobble_U-G, -16_6- | −16 | 16 |
| | | | TTTTTATCCCCATTCGAATA | 6_internal_loop-symmetric_AUUGCA- | | |
| | | | AAGTACTGAGCTATCCCGAA | CUAUUA, -8_1-1_mismatch_C-A, -4- | | |
| | | | TTCAACAATCTTCTATTAGA | >5_10-10_internal_loop- | | |
| | | | TGGCAGCATTGGGATACAGT | symmetric_CUACAGCAUU- | | |
| | | | GTGAAGAGCAGCA | UAUCCCGAAU, 16_6- | | |
| | | | | 6_internal_loop-symmetric_GCUGUA- | | |
| | | | | GAAUAA | | |
| 366 | 70B | 1976 (−22, +16) 0.113.57 | CCCTGGTGTGCCCTCTGATG | −49_1-1_wobble_U-G, -22_6- | −22 | 16 |
| | | | TTTTTATCCCCATTCGAAAA | 6_internal_loop-symmetric_GCCAUC- | | |
| | | | AAGTACTGAGCTATCCCGAA | CAAUCA, -8_1-1_mismatch_C-A, -4- | | |
| | | | TTCAACAATCTTTGCAATCA | >5_10-10_internal_loop- | | |
| | | | ATCAAGCATTGGGATACAGT | symmetric_CUACAGCAUU- | | |
| | | | GTGAAGAGCAGCA | UAUCCCGAAU, 16_6- | | |
| | | | | 6_internal_loop-symmetric_GCUGUA- | | |
| | | | | GAAAAA | | |
| 367 | 70C | 1976 (−22, +26) 0.113.57 | CCCTGGTGTGCCCTCTGATG | −49_1-1_wobble_U-G, -22_6- | −22 | N/A |
| | | | TTTTTCTGGTGATTCTACAA | 6_internal_loop-symmetric_GCCAUC- | | |
| | | | CAGTACTGAGCTATCCCGAA | AAGCGA, -8_1-1_mismatch_C-A, -4- | | |
| | | | TTCAACAATCTTTGCAATAA | >5_10-10_internal_loop- | | |
| | | | GCGAAGCATTGGGATACAG | symmetric_CUACAGCAUU- | | |
| | | | TGTGAAGAGCAGCA | UAUCCCGAAU, 17_1-1_mismatch_C- | | |
| | | | | A, 26_1-1_mismatch_G-G, 27_1- | | |
| | | | | 1_wobble_G-U, 28_4-4_bulge- | | |
| | | | | symmetric_GGAU-CUGG | | |
| 368 | 71A-71C | 1976 (−22, +26) 0.100.70 | CAGGTGCACGAAACCCTGG | -22_6-6_internal_loop- | −22 | N/A |
| | | | TGTGCCCTCTGATGTTTTTCT | symmetric_GCCAUC-AAGCGA, -8_1- | | |
| | | | GGTGATTCTACAACAGTACT | 1_mismatch_C-A, -4->5_10- | | |
| | | | GAGCTATCCCGAATTCAACA | 10_internal_loop- | | |
| | | | ATCTTTGCAATAAGCGAAGC | symmetric_CUACAGCAUU- | | |
| | | | | UAUCCCGAAU, 17_1-1_mismatch_C- | | |
| | | | | A, 26_1-1_mismatch_G-G, 27_1- | | |
| | | | | 1_wobble_G-U, 28_4-4_bulge- | | |
| | | | | symmetric_GGAU-CUGG | | |
| 369 | 71A-71C | 1976 (−22, +26) 0.100.65 | GCACGAAACCCTGGTGTGCC | -22_6-6_internal_loop- | −22 | N/A |
| | | | CTCTGATGTTTTTCTGGTGA | symmetric_GCCAUC-AAGCGA, -8_1- | | |
| | | | TTCTACAACAGTACTGAGCT | 1_mismatch_C-A, -4->5_10- | | |
| | | | ATCCCGAATTCAACAATCTT | 10_internal_loop- | | |
| | | | TGCAATAAGCGAAGCATTG | symmetric_CUACAGCAUU- | | |
| | | | G | UAUCCCGAAU, 17_1-1_mismatch_C- | | |
| | | | | A, 26_1-1_mismatch_G-G, 27_1- | | |
| | | | | 1_wobble_G-U, 28_4-4_bulge- | | |
| | | | | symmetric_GGAU-CUGG | | |
| 370 | 71A-71C | 1976 (−22, +26) 0.100.60 | AAACCCTGGTGTGCCCTCTG | -22_6-6_internal_loop- | −22 | N/A |
| | | | ATGTTTTTCTGGTGATTCTA | symmetric_GCCAUC-AAGCGA, -8_1- | | |
| | | | CAACAGTACTGAGCTATCCC | 1_mismatch_C-A, -4->5_10- | | |
| | | | GAATTCAACAATCTTTGCAA | 10_internal_loop- | | |
| | | | TAAGCGAAGCATTGGGATA | symmetric_CUACAGCAUU- | | |
| | | | C | UAUCCCGAAU, 17_1-1_mismatch_C- | | |
| | | | | A, 26_1-1_mismatch_G-G, 27_1- | | |
| | | | | 1_wobble_G-U, 28_4-4_bulge- | | |
| | | | | symmetric_GGAU-CUGG | | |
| 371 | 71A-71C | 1976 (−22, +26) 0.100.55 | CTGGTGTGCCCTCTGATGTT | -22_6-6_internal_loop- | −22 | N/A |
| | | | TTTCTGGTGATTCTACAACA | symmetric_GCCAUC-AAGCGA, -8_1- | | |
| | | | GTACTGAGCTATCCCGAATT | 1_mismatch_C-A, -4->5_10- | | |
| | | | CAACAATCTTTGCAATAAGC | 10_internal_loop- | | |
| | | | GAAGCATTGGGATACAGTG | symmetric_CUACAGCAUU- | | |
| | | | T | UAUCCCGAAU, 17_1-1_mismatch_C- | | |
| | | | | A, 26_1-1_mismatch_G-G, 27_1- | | |
| | | | | 1_wobble_G-U, 28_4-4_bulge- | | |
| | | | | symmetric_GGAU-CUGG | | |
| 372 | 71A-71C | 1976 (−22, +26) 0.100.50 | GTGCCCTCTGATGTTTTTCT | -22_6-6_internal_loop- | −22 | N/A |
| | | | GGTGATTCTACAACAGTACT | symmetric_GCCAUC-AAGCGA, -8_1- | | |
| | | | GAGCTATCCCGAATTCAACA | 1_mismatch_C-A, -4->5_10- | | |
| | | | ATCTTTGCAATAAGCGAAGC | 10_internal_loop- | | |
| | | | ATTGGGATACAGTGTGAAA | symmetric_CUACAGCAUU- | | |
| | | | A | UAUCCCGAAU, 17_1-1_mismatch_C- | | |
| | | | | A, 26_1-1_mismatch_G-G, 27_1- | | |
| | | | | 1_wobble_G-U, 28_4-4_bulge- | | |
| | | | | symmetric_GGAU-CUGG | | |
| 373 | 71A-71C | 1976 (−22, +26) 0.113.57 | CCCTGGTGTGCCCTCTGATG | −49_1-1_wobble_U-G, -22_6- | −22 | N/A |
| | | | TTTTTCTGGTGATTCTACAA | 6_internal_loop-symmetric_GCCAUC- | | |
| | | | CAGTACTGAGCTATCCCGAA | AAGCGA, -8_1-1_mismatch_C-A, -4- | | |
| | | | TTCAACAATCTTTGCAATAA | >5_10-10_internal_loop- | | |
| | | | GCGAAGCATTGGGATACAG | symmetric_CUACAGCAUU- | | |
| | | | TGTGAAGAGCAGCA | UAUCCCGAAU, 17_1-1_mismatch_C- | | |
| | | | | A, 26_1-1_mismatch_G-G, 27_1- | | |
| | | | | 1_wobble_G-U, 28_4-4_bulge- | | |
| | | | | symmetric_GGAU-CUGG | | |
| 374 | 72A-72C | 1976 (−22, +34) 0.100.65 | GCACGAAACCCTGGTGTGCC | -22 6-6_internal_loop- | −22 | 34 |
| | | | CTCTGTACAAATTATCCCCA | symmetric_GCCAUC-AAGCGA, -8_1- | | |
| | | | TTCTACAACAGTACTGAGCT | 1_mismatch_C-A, -4->5_10- | | |
| | | | ATCCCGAATTCAACAATCTT | 10_internal_loop- | | |
| | | | TGCAATAAGCGAAGCATTG | symmetric_CUACAGCAUU- | | |
| | | | G | UAUCCCGAAU, 17_1-1_mismatch_C- | | |
| | | | | A, 34_6-6_internal_loop- | | |
| | | | | symmetric_AAACAU-UACAAA | | |
| 375 | 72A-72C | 1976 (−22, +32) 0.100.65 | GCACGAAACCCTGGTGTGCC | -22_6-6_internal_loop- | −22 | 32 |
| | | | CTCTGATCAAAAAATCCCCA | symmetric_GCCAUC-AAGCGA, -8_1- | | |
| | | | TTCTACAACAGTACTGAGCT | 1_mismatch_C-A, -4->5_10- | | |
| | | | ATCCCGAATTCAACAATCTT | 10_internal_loop- | | |
| | | | TGCAATAAGCGAAGCATTG | symmetric_CUACAGCAUU- | | |
| | | | G | UAUCCCGAAU, 17_1-1_mismatch_C- | | |
| | | | | A, 32_6-6_internal_loop- | | |
| | | | | symmetric_AAAAAC-CAAAAA | | |
| 376 | 72A-72C | 1976 (−22, +30) 0.100.65 | GCACGAAACCCTGGTGTGCC | -22_6-6_internal_loop- | −22 | 30 |
| | | | CTCTGATGTAAAATGCCCCA | symmetric_GCCAUC-AAGCGA, -8_1- | | |
| | | | TTCTACAACAGTACTGAGCT | 1_mismatch_C-A, -4->5_10- | | |
| | | | ATCCCGAATTCAACAATCTT | 10_internal_loop- | | |
| | | | TGCAATAAGCGAAGCATTG | symmetric_CUACAGCAUU- | | |
| | | | G | UAUCCCGAAU, 17_1-1_mismatch_C- | | |
| | | | | A, 30_6-6_internal_loop- | | |
| | | | | symmetric_AUAAAA-AAAAUG | | |
| 377 | 72A-72C | 1976 (−22, +28) 0.100.65 | GCACGAAACCCTGGTGTGCC | -22_6-6_internal_loop- | −22 | 28 |
| | | | CTCTGATGTTTAATGGGCCA | symmetric_GCCAUC-AAGCGA, -8_1- | | |
| | | | TTCTACAACAGTACTGAGCT | 1_mismatch_C-A, -4->5_10- | | |
| | | | ATCCCGAATTCAACAATCTT | 10_internal_loop- | | |
| | | | TGCAATAAGCGAAGCATTG | symmetric_CUACAGCAUU- | | |
| | | | G | UAUCCCGAAU, 17_1-1_mismatch_C- | | |
| | | | | A, 28_6-6_internal_loop- | | |
| | | | | symmetric_GGAUAA-AAUGGG | | |
| 378 | 72A-72C | 1976 (−22, +26) V2 | GCACGAAACCCTGGTGTGCC | -22_6-6_internal_loop- | −22 | N/A |
| | | 0.100.65 | CTCTGATGTTTTTTGGGTGA | symmetric_GCCAUC-AAGCGA, -8_1- | | |
| | | | TTCTACAACAGTACTGAGCT | 1_mismatch_C-A, -4->5_10- | | |
| | | | ATCCCGAATTCAACAATCTT | 10_internal_loop- | | |
| | | | TGCAATAAGCGAAGCATTG | symmetric_CUACAGCAUU- | | |
| | | | G | UAUCCCGAAU, 17_1-1_mismatch_C- | | |
| | | | | A, 26_1-1_mismatch_G-G, 27_1- | | |
| | | | | 1_wobble_G-U, 28_4-4_bulge- | | |
| | | | | symmetric_GGAU-UGGG | | |
| 379 | 72A-72C | 1976 (−22, +26) 0.100.65 | GCACGAAACCCTGGTGTGCC | -22_6-6_internal_loop- | −22 | N/A |
| | | | CTCTGATGTTTTTCTGGTGA | symmetric_GCCAUC-AAGCGA, -8_1- | | |
| | | | TTCTACAACAGTACTGAGCT | 1_mismatch_C-A, -4->5_10- | | |
| | | | ATCCCGAATTCAACAATCTT | 10_internal_loop- | | |
| | | | TGCAATAAGCGAAGCATTG | symmetric_CUACAGCAUU- | | |
| | | | G | UAUCCCGAAU, 17_1-1_mismatch_C- | | |
| | | | | A, 26_1-1_mismatch_G-G, 27_1- | | |
| | | | | 1_wobble_G-U, 28_4-4_bulge- | | |
| | | | | symmetric_GGAU-CUGG | | |
| 380 | 72A-72C | 1976 (−22, +24) 0.100.65 | GCACGAAACCCTGGTGTGCC | -22_6-6_internal_loop- | −22 | 24 |
| | | | CTCTGATGTTTTTATGGTGT | symmetric_GCCAUC-AAGCGA, -8_1- | | |
| | | | ATCTACAACAGTACTGAGCT | 1_mismatch_C-A, -4->5_10- | | |
| | | | ATCCCGAATTCAACAATCTT | 10_internal_loop- | | |
| | | | TGCAATAAGCGAAGCATTG | symmetric_CUACAGCAUU- | | |
| | | | G | UAUCCCGAAU, 17_1-1_mismatch_C- | | |
| | | | | A, 24_6-6_internal_loop- | | |
| | | | | symmetric_AUGGGG-GGUGUA | | |
| 381 | 72A-72C | 1976 (−22, +22) 0.100.65 | GCACGAAACCCTGGTGTGCC | -22_6-6_internal_loop- | −22 | 22 |
| | | | CTCTGATGTTTTTATCCGGT | symmetric_GCCAUC-AAGCGA, -8_1- | | |
| | | | AGGTACAACAGTACTGAGC | 1_mismatch_C-A, -4->5_10- | | |
| | | | TATCCCGAATTCAACAATCT | 10_internal_loop- | | |
| | | | TTGCAATAAGCGAAGCATTG | symmetric_CUACAGCAUU- | | |
| | | | G | UAUCCCGAAU, 17_1-1_mismatch_C- | | |
| | | | | A, 22_6-6_internal_loop- | | |
| | | | | symmetric_GAAUGG-GGUAGG | | |
| 382 | 72A-72C | 1976 (−22, +20) 0.100.65 | GCACGAAACCCTGGTGTGCC | -22_6-6_internal_loop- | −22 | 20 |
| | | | CTCTGATGTTTTTATCCCCTA | symmetric_GCCAUC-AAGCGA, -8_1- | | |
| | | | GGATCAACAGTACTGAGCT | 1_mismatch_C-A, -4->5_10- | | |
| | | | ATCCCGAATTCAACAATCTT | 10_internal_loop- | | |
| | | | TGCAATAAGCGAAGCATTG | symmetric_CUACAGCAUU- | | |
| | | | G | UAUCCCGAAU, 17_1-1_mismatch_C- | | |
| | | | | A, 20_6-6_internal_loop- | | |
| | | | | symmetric_UAGAAU-UAGGAU | | |
| 383 | 73 | 1976 (−25, +26) 0.100.65 | GCACGAAACCCTGGTGTGCC | -25_6-6_internal_loop- | −25 | N/A |
| | | | CTCTGATGTTTTTTGGGTGA | symmetric_GCUGCC-CAAUCA, -8_1- | | |
| | | | TTCTACAACAGTACTGAGCT | 1_mismatch_C-A, -4->5_10- | | |
| | | | ATCCCGAATTCAACAATCTT | 10_internal_loop- | | |
| | | | TGCAATGATCAATCAATTGG | symmetric_CUACAGCAUU- | | |
| | | | | UAUCCCGAAU, 17_1-1_mismatch_C- | | |
| | | | | A, 26_1-1_mismatch_G-G, 27_1- | | |
| | | | | 1_wobble_G-U, 28_4-4_bulge- | | |
| | | | | symmetric_GGAU-UGGG | | |
| 384 | 73 | 1976 (−22, +26) 0.100.65 | GCACGAAACCCTGGTGTGCC | -22_6-6_internal_loop- | −22 | N/A |
| | | | CTCTGATGTTTTTCTGGTGA | symmetric_GCCAUC-AAGCGA, -8_1- | | |
| | | | TTCTACAACAGTACTGAGCT | 1_mismatch_C-A, -4->5_10- | | |
| | | | ATCCCGAATTCAACAATCTT | 10_internal_loop- | | |
| | | | TGCAATAAGCGAAGCATTG | symmetric_CUACAGCAUU- | | |
| | | | G | UAUCCCGAAU, 17_1-1_mismatch_C- | | |
| | | | | A, 26_1-1_mismatch_G-G, 27_1- | | |
| | | | | 1_wobble_G-U, 28_4-4_bulge- | | |
| | | | | symmetric_GGAU-CUGG | | |
| 385 | 73 | 1976 (−20, +26) 0.100.65 | GCACGAAACCCTGGTGTGCC | -20_6-6_internal_loop- | −20 | N/A |
| | | | CTCTGATGTTTTTTGGGTGA | symmetric_CAUCAU-CAAAGC, -8_1- | | |
| | | | TTCTACAACAGTACTGAGCT | 1_mismatch_C-A, -4->5_10- | | |
| | | | ATCCCGAATTCAACAATCTT | 10_internal_loop- | | |
| | | | TGCACAAAGCGCAGCATTG | symmetric_CUACAGCAUU- | | |
| | | | G | UAUCCCGAAU, 17_1-1_mismatch_C- | | |
| | | | | A, 26_1-1_mismatch_G-G, 27_1- | | |
| | | | | 1_wobble_G-U, 28_4-4_bulge- | | |
| | | | | symmetric_GGAU-UGGG | | |
| 386 | 73 | 1976 (−18, +26) 0.100.65 | GCACGAAACCCTGGTGTGCC | -18_6-6_internal_loop- | −18 | N/A |
| | | | CTCTGATGTTTTTTGGGTGA | symmetric_UCAUUG-GUCAAU, -8_1- | | |
| | | | TTCTACAACAGTACTGAGCT | 1_mismatch_C-A, -4->5_10- | | |
| | | | ATCCCGAATTCAACAATCTT | 10_internal_loop- | | |
| | | | TGGTCAATTGGCAGCATTGG | symmetric_CUACAGCAUU- | | |
| | | | | UAUCCCGAAU, 17_1-1_mismatch_C- | | |
| | | | | A, 26_1-1_mismatch_G-G, 27_1- | | |
| | | | | 1_wobble_G-U, 28_4-4_bulge- | | |
| | | | | symmetric_GGAU-UGGG | | |
| 387 | 73 | 1976 (−16, +26) 0.100.65 | GCACGAAACCCTGGTGTGCC | -16_6-6_internal_loop- | −16 | N/A |
| | | | CTCTGATGTTTTTTGGGTGA | symmetric_AUUGCA-AAGUCA, -8_1- | | |
| | | | TTCTACAACAGTACTGAGCT | 1_mismatch_C-A, -4->5_10- | | |
| | | | ATCCCGAATTCAACAATCTT | 10_internal_loop- | | |
| | | | AAGTCAGATGGCAGCATTG | symmetric_CUACAGCAUU- | | |
| | | | G | UAUCCCGAAU, 17_1-1_mismatch_C- | | |
| | | | | A, 26_1-1_mismatch_G-G, 27_1- | | |
| | | | | 1_wobble_G-U, 28_4-4_bulge- | | |
| | | | | symmetric_GGAU-UGGG | | |
| 388 | 73 | 1976 (−14, +26) 0.100.65 | GCACGAAACCCTGGTGTGCC | −14_6-6_internal_loop- | −14 | N/A |
| | | | CTCTGATGTTTTTTGGGTGA | symmetric_UGCAAA-AAAAGU, -8_1- | | |
| | | | TTCTACAACAGTACTGAGCT | 1_mismatch_C-A, -4->5_10- | | |
| | | | ATCCCGAATTCAACAATCAA | 10_internal_loop- | | |
| | | | AAGTATGATGGCAGCATTG | symmetric_CUACAGCAUU- | | |
| | | | G | UAUCCCGAAU, 17_1-1_mismatch_C- | | |
| | | | | A, 26_1-1_mismatch_G-G, 27_1- | | |
| | | | | 1_wobble_G-U, 28_4-4_bulge- | | |
| | | | | symmetric_GGAU-UGGG | | |
| 389 | 74 | 1700 (−20, +26) 0.113.57 | CCCTGGTGTGCCCTCTGATG | −49_1-1_wobble_U-G, -20_6- | −20 | 26 |
| | | | TTTTTTAGGGGATTCTACCG | 6_internal_loop-symmetric_CAUCAU- | | |
| | | | CAGTACTACCCGATCCCGTA | UACUAC, 0_1-1_mismatch_A-C, 2_1- | | |
| | | | GTCAGCAATCTTTGCATACT | 1_mismatch_C-C, 5_1-1_wobble_U-G, | | |
| | | | ACGCAGCATTGGGATACAG | 7_3−3_bulge-symmetric_CUC-ACC, | | |
| | | | TGTGAAGAGCAGCA | 18_1-1_mismatch_U-C, 26_6- | | |
| | | | | 6_internal_loop-symmetric_GGGGAU- | | |
| | | | | UAGGGG | | |
| 390 | 75A | 860 (−8, +24) 0.113.57 | CCCTGGTGTGCCCTCTGATG | −49_1-1_wobble_U-G, -8_6- | −8 | 24 |
| | | | TTTTTATGGAAAGTCTACAG | 6_internal_loop-symmetric_GAUUGC- | | |
| | | | CAGTACAGAGGACTGCCGA | CUUUGA, −3_1-1_wobble_U-G, -2_1- | | |
| | | | GGTCACTTTGATTTGCAATG | 1_mismatch_A-A, 0_1-1_mismatch_A- | | |
| | | | ATGGCAGCATTGGGATACA | C, 4_1-1_mismatch_U-C, 6_1- | | |
| | | | GTGTGAAGAGCAGCA | 1_mismatch_G-G, 10_1-1_mismatch_A- | | |
| | | | | A, 24_6-6_internal_loop- | | |
| | | | | symmetric_AUGGGG-GGAAAG | | |
| 391 | 75B | 860 (−12, +30) 0.113.57 | CCCTGGTGTGCCCTCTGATG | −49_1-1_wobble_U-G, -12_6- | −12 | 30 |
| | | | TATGTTCCCCCATTCTACAG | 6_internal_loop-symmetric_CAAAGA- | | |
| | | | CAGTACAGAGGACTGCCGA | CGCUAA, -8_1-1_mismatch_C-C, | | |
| | | | GGTCACCAACGCTAACAAT | symmetric3 1-1_wobble U-G, -2_1- | | |
| | | | GATGGCAGCATTGGGATAC | 1_mismatch_A-A, 0_1-1_mismatch_A- | | |
| | | | AGTGTGAAGAGCAGCA | C, 4_1-1_mismatch_U-C, 6_1- | | |
| | | | | 1_mismatch_G-G, 10_1-1_mismatch_A- | | |
| | | | | A, 30_6-6_internal_loop- | | |
| | | | | symmetric_AUAAAA-AUGUUC | | |
| 392 | 75C | 860 (−16, +26) 0.113.57 | CCCTGGTGTGCCCTCTGATG | −49_1-1_wobble_U-G, -16_6- | −16 | 26 |
| | | | TTTTTCGGGAGATTCTACAG | 6_internal_loop-symmetric_AUUGCA- | | |
| | | | CAGTACAGAGGACTGCCGA | GUCUAG, -8_1-1_mismatch_C-C, | | |
| | | | GGTCACCAATCTTGTCTAGG | −3_1-1_wobble_U-G, -2_1- | | |
| | | | ATGGCAGCATTGGGATACA | 1_mismatch_A-A, 0_1-1_mismatch_A- | | |
| | | | GTGTGAAGAGCAGCA | C, 4_1-1_mismatch_U-C, 6_1- | | |
| | | | | 1_mismatch_G-G, 10_1-1_mismatch_A- | | |
| | | | | A, 26_6-6_internal_loop- | | |
| | | | | symmetric_GGGGAU-CGGGAG | | |
| 393 | 75D | 860 (−20, +26) 0.113.57 | CCCTGGTGTGCCCTCTGATG | −49_1-1_wobble_U-G, -20_6- | −20 | 26 |
| | | | TTTTTTAGGGGATTCTACAG | 6_internal_loop-symmetric_CAUCAU- | | |
| | | | CAGTACAGAGGACTGCCGA | UACUAC, -8_1-1_mismatch_C-C, | | |
| | | | GGTCACCAATCTTTGCATAC | −3_1-1_wobble_U-G, -2_1- | | |
| | | | TACGCAGCATTGGGATACA | 1_mismatch_A-A, 0_1-1_mismatch_A- | | |
| | | | GTGTGAAGAGCAGCA | C, 4_1-1_mismatch_U-C, 6_1- | | |
| | | | | 1_mismatch_G-G, 10_1-1_mismatch_A- | | |
| | | | | A, 26_6-6_internal_loop- | | |
| | | | | symmetric_GGGGAU-UAGGGG | | |
| 394 | 75E | 860 (−22, +26) 0.113.57 | CCCTGGTGTGCCCTCTGATG | −49_1-1_wobble_U-G, -22_6- | −22 | N/A |
| | | | TTTTTCTACAGATTCTACAG | 6_internal_loop-symmetric_GCCAUC- | | |
| | | | CAGTACAGAGGACTGCCGA | AACCUG, -8_1-1_mismatch_C-C, | | |
| | | | GGTCACCAATCTTTGCAATA | −3_1-1_wobble_U-G, -2_1- | | |
| | | | ACCTGAGCATTGGGATACA | 1_mismatch_A-A, 0_1-1_mismatch_A- | | |
| | | | GTGTGAAGAGCAGCA | C, 4_1-1_mismatch_U-C, 6_1- | | |
| | | | | 1_mismatch_G-G, 10_1-1_mismatch_A- | | |
| | | | | A, 26_2-2_bulge-symmetric_GG-AG, | | |
| | | | | 29_3−3_bulge-symmetric_GAU-CUA | | |
| 395 | 76 | 2108 (−10, +16) 0.113.57 | CCCTGGTGTGCCCTCTGATG | −49_1-1_wobble_U-G, -10_6- | −10 | 16 |
| | | | TTTTTATCCCCATTCGCCTG | 6_internal_loop-symmetric_AAGAUU- | | |
| | | | AGGTACTGACCAATCCCGTA | CUAGGC, -6_1-1_wobble_G-U, 0_1- | | |
| | | | GTTAGCCTAGGCTGCAATGA | 1_mismatch_A-C, 2_1-1_mismatch_C- | | |
| | | | TGGCAGCATTGGGATACAGT | C, 7_1-1_mismatch_C-C, 15_1- | | |
| | | | GTGAAGAGCAGCA | 1_wobble_U-G, 16_6-6_internal_loop- | | |
| | | | | symmetric_GCUGUA-GCCUGA | | |
| 5 | 77 | 610 (−14, +26) 0.113.57 | CCCTGGTGTGCCCTCTGATG | −49_1-1_wobble_U-G, −14_6- | −14 | 26 |
| | | | TTTTTTAGGGGATTCTACAT | 6_internal_loop-symmetric_UGCAAA- | | |
| | | | CTGTAGTGAGCAATTCCGTG | AAACGU, -4_1-1_mismatch_C-C, | | |
| | | | CTCAGCAATCAAACGTATGA | −3_1-1_wobble_U-G, 0_1- | | |
| | | | TGGCAGCATTGGGATACAGT | 1_mismatch_A-C, 2_1-1_mismatch_C- | | |
| | | | GTGAAGAGCAGCA | U, 11_1-1_mismatch_G-G, 15_1- | | |
| | | | | 1_mismatch_U-U, 17_1-1_mismatch_C- | | |
| | | | | U, 26_6-6_internal_loop- | | |
| | | | | symmetric_GGGGAU-UAGGGG | | |
| 23 | 77 | 871 (−16, +24) 0.113.57 | CCCTGGTGTGCCCTCTGATG | −49_1-1_wobble_U-G, -16_6- | −16 | 24 |
| | | | TTTTTATGGGGTGTCTACAG | 6_internal_loop-symmetric_AUUGCA- | | |
| | | | TAGGACTGAGCACTGCCGA | GAGUCG, −7_1-1_wobble_U-G, -6_1- | | |
| | | | GCTGGGCAATCTTGAGTCGG | 1_mismatch_G-G, -4_0-1_bulge- | | |
| | | | ATGGCAGCATTGGGATACA | asymmetric_-C, -2_1-0_bulge- | | |
| | | | GTGTGAAGAGCAGCA | asymmetric_A-, 0_1-1_mismatch_A-C, | | |
| | | | | 4_1-1_mismatch_U-C, 13_1- | | |
| | | | | 1_mismatch_A-G, 16_1-1_wobble_G-U, | | |
| | | | | 24_6-6_internal_loop- | | |
| | | | | symmetric_AUGGGG-GGGGUG | | |
| 50 | 77 | 919 (−12, +24) 0.113.57 | CCCTGGTGTGCCCTCTGATG | −49_1-1_wobble_U-G, -12_6- | −12 | 24 |
| | | | TTTTTATGAGGCGTCTACAG | 6_internal_loop-symmetric_CAAAGA- | | |
| | | | CAGTTCGGAGGAATCCCGA | GACUAA, -3_1-1_wobble_U-G, -2_1- | | |
| | | | GGTCAGCAAGACTAACAAT | 1_mismatch_A-A, 0_1-1_mismatch_A- | | |
| | | | GATGGCAGCATTGGGATAC | C, 2_1-1_mismatch_C-C, 6_1- | | |
| | | | AGTGTGAAGAGCAGCA | 1_mismatch_G-G, 10_3−3_bulge- | | |
| | | | | symmetric_AGU-UCG, 24_6- | | |
| | | | | 6_internal_loop-symmetric_AUGGGG- | | |
| | | | | GAGGCG | | |
| 228 | 77 | 610 (−10, +34) 0.92.60 | AAACCCTGGTGTGCCCTCTG | -10_6-6_internal_loop- | −10 | 34 |
| | | | TCCAAATTATCCCCATTCTA | symmetric_AAGAUU-UCAGAA, -4_1- | | |
| | | | CATCTGTAGTGAGCAATTCC | 1_mismatch_C-C, −3_1-1_wobble U-G, | | |
| | | | GTGCTCAGCTCAGAATGCAA | 0_1-1_mismatch_A-C, 2_1- | | |
| | | | TGATGGCAGCAT | 1_mismatch_C-U, 11_1-1_mismatch_G- | | |
| | | | | G, 15_1-1_mismatch_U-U, 17_1- | | |
| | | | | 1_mismatch_C-U, 34_6- | | |
| | | | | 6_internal_loop-symmetric_AAACAU- | | |
| | | | | UCCAAA | | |
| 312 | 77 | 871 (−16, +32) 0.98.55 | CTGGTGTGCCCTCTGATCCA | -16_6-6_internal_loop- | −16 | 32 |
| | | | AGCATCCCCATTCTACAGTA | symmetric_AUUGCA-GAGUCG, −7_1- | | |
| | | | GGACTGAGCACTGCCGAGC | 1_wobble_U-G, -6_1-1_mismatch_G-G, | | |
| | | | TGGGCAATCTTGAGTCGGAT | -4_0-1_bulge-asymmetric_-C, -2_1- | | |
| | | | GGCAGCATTGGGATACAGT | 0_bulge-asymmetric_A-, 0_1- | | |
| | | | | 1_mismatch_A-C, 4_1-1_mismatch_U- | | |
| | | | | C, 13_1-1_mismatch_A-G, 16 1- | | |
| | | | | 1_wobble_G-U, 32_6-6_internal_loop- | | |
| | | | | symmetric_AAAAAC-CCAAGC | | |
| 354 | 77 | 919 (−14, +22) 0.96.50 | GTGCCCTCTGATGTTTTTAT | −14_6-6_internal_loop- | −14 | 22 |
| | | | CCGGCGAGTACAGCAGTTC | symmetric_UGCAAA-CUAAGU, −3_1- | | |
| | | | GGAGGAATCCCGAGGTCAG | 1_wobble_U-G, -2_1-1_mismatch_A-A, | | |
| | | | CAATCCTAAGTATGATGGCA | 0_1-1_mismatch_A-C, 2_1- | | |
| | | | GCATTGGGATACAGTGTG | 1_mismatch_C-C, 6_1-1_mismatch_G- | | |
| | | | | G, 10_3−3_bulge-symmetric_AGU- | | |
| | | | | UCG, 22_6-6_internal_loop- | | |
| | | | | symmetric_GAAUGG-GGCGAG | | |

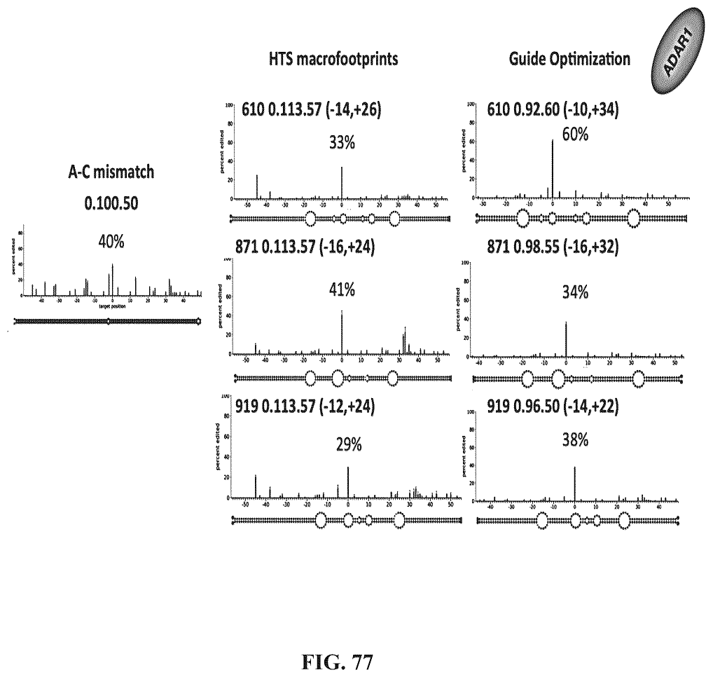

As shown in FIG. 77, engineering of guide610, guide871 and guide919 produced a significant increase in editing efficiency. Thus, this example demonstrates that guide RNAs selected via high throughput screening against LRRK2 can be systematically engineered to dramatically improve their editing efficiencies by modulating the positioning of the barbell macro-footprint within the guide-target RNA scaffold.

### Example 10

In Vitro Screening of LRRK2 gRNAs Selected Using HTS

This example describes construction of an scAAV vector for in vitro screening of LRRK2 engineered guide RNAs selected using a high throughput screen (see EXAMPLE 1) and/or engineered as described in EXAMPLE 9. For this example, count919 (−14, 22)—SEQ ID NO: 354, count871 (−16, 32)—SEQ ID NO: 312, count2397 (−14, 28)—SEQ ID NO: 351, count610 (−14, 34)—SEQ ID NO: 228 and count1976 (−22, 26)—SEQ ID NO: 369 were evaluated.

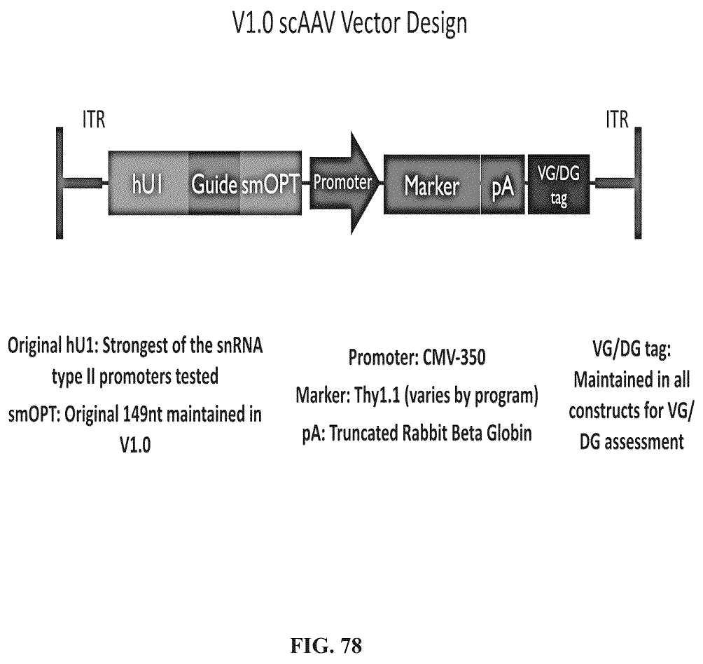

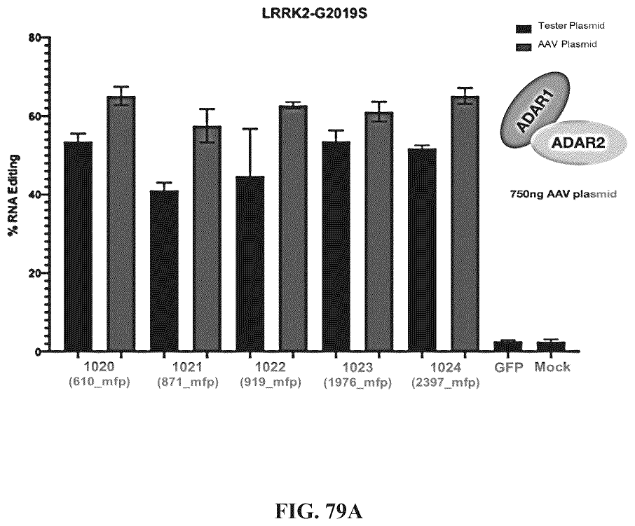

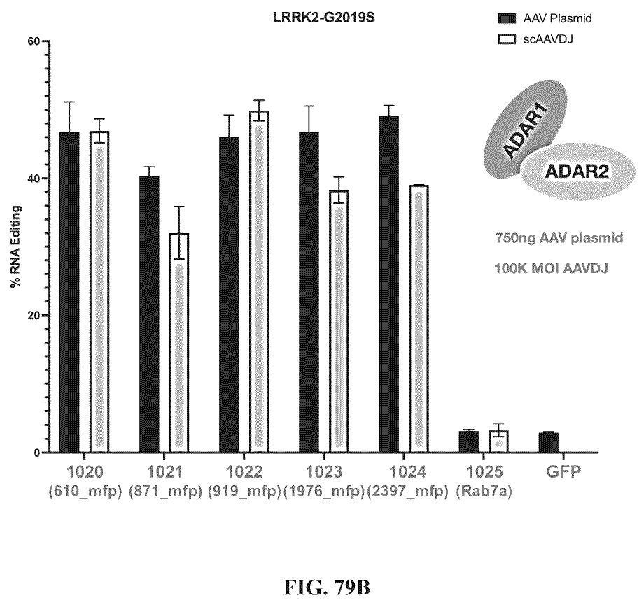

Each engineered guide RNA was cloned into an scAAV vector, as shown in FIG. 78, having a human U1 promotor (TAAGGACCAGCTTCTTTGGGAGAGAACAGACGCAGGGGCGGGAGGGAAAAAGGG AGAGGCAGACGTCACTTCCTCTTGGCGACTCTGGCAGCAGATTGGTCGGTTGAGTG GCAGAAAGGCAGACGGGGACTGGGCAAGGCACTGTCGGTGACATCACGGACAGGG CGACTTCTATGTAGATGAGGCAGCGCAGAGGCTGCTGCTTCGCCACTTGCTGCTTCG CCACGAAGGGAGTTCCCGTGCCCTGGGAGCGGGTTCAGGACCGCTGATCGGAAGTG AGAATCCCAGCTGTGTGTCAGGGCTGGAAAGGGCTCGGGAGTGCGCGGGGCAAGT GACCGTGTGTGTAAAGAGTGAGGCGTATGAGGCTGTGTCGGGGCAGAGCCCGAAG ATCTC)—SEQ ID NO: 396 and an SmOPT sequence (AATTTTTGGAG)—SEQ ID NO: 397 flanking each guide. Each vector was transfected into HEK293 cells, and the percent RNA editing facilitated by each engineered guide RNA via ADAR1+ADAR2 was compared to control. As shown in FIG. 79A, each engineered guide RNA transfected facilitated higher levels of editing relative to the control. Each variant was then packaged into an scAAV virus, and the ability of each guide to facilitate editing via ADAR1+ADAR2 after transduction was determined. As shown in FIG. 79B, each guide RNA displayed comparable editing when packaged as an scAAV virus via transduction as when transfected as an AAV plasmid. Following differentiation, all cell lines selected as LRRK2 in vitro models display key features of neuronal development including neurite outgrowth and cell-to-cell connections. As such, this example demonstrates repair of neuronal development in the in vitro cell model upon transfection with the scAAV vector containing engineered guide RNAs.

### Example 11

### Editing of LRRK2 by Engineered Guide RNA Using a Broken GFP Reporter

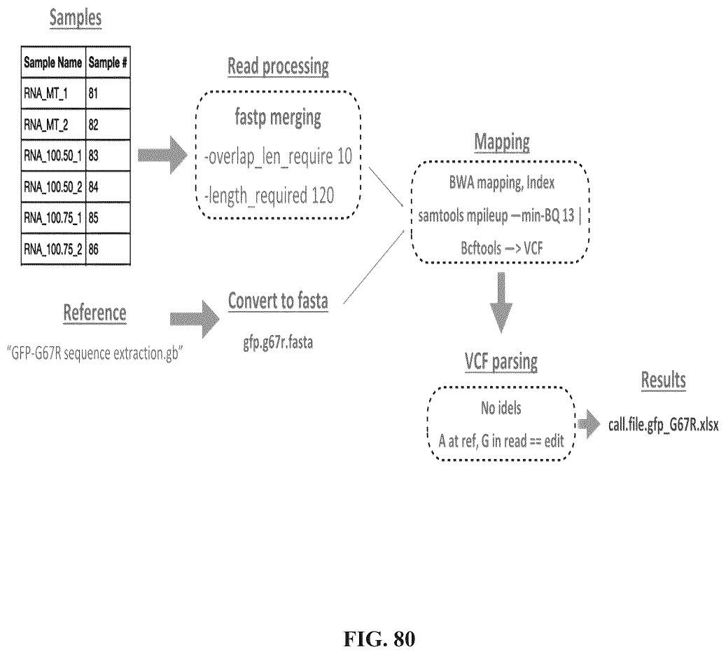

100 nucleotide engineered guide RNAs with an A/C mismatch at positions 25, 50, and 75 with LRRK2 guide mimicry or barbells at different positions were tested in K562 cells expressing a GFP-G67R reporter. FIG. 80 depicts a workflow for screening exemplary guide RNAs targeting LRRK2 in a broken GFP reporter system. The cells were selected by puromycin to enrich for plasmid and guide integration. The WT ADAR results were from cells captured following 14 days of puro selection and for the ADAR2 overexpression (with a weak constitutive promoter, PGK) 21 days of selection. The editing was assessed by NGS sequencing on the iSeq instrument.

TABLE 8 below recites the engineered guide RNA sequences utilized in this example. While the engineered guide RNA sequences in TABLE 8 are provided as DNA sequences with a T substituted for each U, the corresponding RNA sequences are also encompassed herein.

TABLE 8
Engineered LRRK2 Guide RNA Sequences utilized in Broken GFP Reporter
System

| SEQ | | | |
|---|---|---|---|
| ID | | Guide | |
| NO | FIG. | Name | Sequence |
| 398 | 81 | 100.75_AC_CO1 | GCCTTCGGGCATGGCGGACTTGAAGAAGTCGTGCTGCTTC |
| | | | ATGTGGTCGGGGTAGCGGCTGAAGCACTGCACTCCGTAG |
| | | | GTCAGGGTCGTCACGAGGGTC |
| 399 | 81 | 100.50_AC_CO1 | AAGTCGTGCTGCTTCATGTGGTCGGGGTAGCGGCTGAAGC |
| | | | ACTGCACTCCGTAGGTCAGGGTCGTCACGAGGGTCGGCC |
| | | | AGGGCACGGGCAGCTTGCCGG |
| 400 | 81 | 100.25_AC_CO1 | GGTAGCGGCTGAAGCACTGCACTCCGTAGGTCAGGGTCG |
| | | | TCACGAGGGTCGGCCAGGGCACGGGCAGCTTGCCGGTGG |
| | | | TGCAGATGAACTTCAGGGTCAG |
| 401 | 81 | 0.100.75_(LRRK2_1976_ | GCCTTCGGGCATGGCGGACTTGAAGAAGTCGTGCTGCTTC |
| | | mimicry) | ATGTGGTCGGGGTAGCGCCTGAAGCACTGGACAGCCCTC |
| | | | GTCTGGGTCGTCACGAGGGTC |
| 402 | 81 | 0.100.75_(LRRK2_919_ | GCCTTCGGGCATGGCGGACTTGAAGAAGTCGTGCTGCTTC |
| | | mimicry) | ATGTGGTCGGGGTAGCGGCTGATCAACTCCACAGCCAAG |
| | | | GTCAGGGTCGTCACGAGGGTC |
| 403 | 81 | 0.100.75_(LRRK2_871_ | GCCTTCGGGCATGGCGGACTTGAAGAAGTCGTGCTGCTTC |
| | | mimicry) | ATGTGGTCGGGGTAGCGGCTGTAGCACTGCCCTCCCATCC |
| | | | ACAGGGTCGTCACGAGGGTC |
| 404 | 81 | 0.100.75_(LRRK2_860_ | GCCTTCGGGCATGGCGGACTTGAAGAAGTCGTGCTGCTTC |
| | | mimicry) | ATGTGGTCGGGGTAGCGGCTGAAGGACTCGTCTCCCAAG |
| | | | GTCTGGGTCGTCACGAGGGTC |
| 405 | 81 | 0.100.50_(LRRK2_1976_ | AAGTCGTGCTGCTTCATGTGGTCGGGGTAGCGCCTGAAGC |
| | | mimicry) | ACTGGACAGCCCTCGTCTGGGTCGTCACGAGGGTCGGCC |
| | | | AGGGCACGGGCAGCTTGCCGG |
| 406 | 81 | 0.100.50_(LRRK2_919_ | AAGTCGTGCTGCTTCATGTGGTCGGGGTAGCGGCTGATCA |
| | | mimicry) | ACTCCACAGCCAAGGTCAGGGTCGTCACGAGGGTCGGCC |
| | | | AGGGCACGGGCAGCTTGCCGG |
| 407 | 81 | 0.100.50_(LRRK2_871_ | AAGTCGTGCTGCTTCATGTGGTCGGGGTAGCGGCTGTAGC |
| | | mimicry) | ACTGCCCTCCCATCCACAGGGTCGTCACGAGGGTCGGCCA |
| | | | GGGCACGGGCAGCTTGCCGG |
| 408 | 81 | 0.100.50_(LRRK2_860_ | AAGTCGTGCTGCTTCATGTGGTCGGGGTAGCGGCTGAAG |
| | | mimicry) | GACTCGTCTCCCAAGGTCTGGGTCGTCACGAGGGTCGGCC |
| | | | AGGGCACGGGCAGCTTGCCGG |
| 409 | 81 | −30, +6_0.100.25 | GGTAGCGGCTGAACGTGACCACTCCGTAGGTCAGGGTCG |
| | | | TCACGAGGGTCGGCCTCCCGTCGGGCAGCTTGCCGGTGGT |
| | | | GCAGATGAACTTCAGGGTCAG |
| 410 | 81 | −30, +5_0.100.25 | GGTAGCGGCTGAAGGTGACGACTCCGTAGGTCAGGGTCG |
| | | | TCACGAGGGTCGGCCTCCCGTCGGGCAGCTTGCCGGTGGT |
| | | | GCAGATGAACTTCAGGGTCAG |
| 411 | 81 | −6, +30_0.100.75 | GCCTTCGGGCATGGCGGACTTGAAGAAGTCGTGCTGCTTG |
| | | | TACACGTCGGGGTAGCGGCTGAAGCACTGCACTCCGTAG |
| | | | GAGTCCCTCGTCACGAGGGTC |
| 412 | 81 | −6, +30_0.100.50 | AAGTCGTGCTGCTTGTACACGTCGGGGTAGCGGCTGAAG |
| | | | CACTGCACTCCGTAGGAGTCCCTCGTCACGAGGGTCGGCC |
| | | | AGGGCACGGGCAGCTTGCCGG |
| 413 | 81 | −5, +30_0.100.75 | GCCTTCGGGCATGGCGGACTTGAAGAAGTCGTGCTGCTTG |
| | | | TACACGTCGGGGTAGCGGCTGAAGCACTGCACTCCGTAG |
| | | | TAATCCGTCGTCACGAGGGTC |

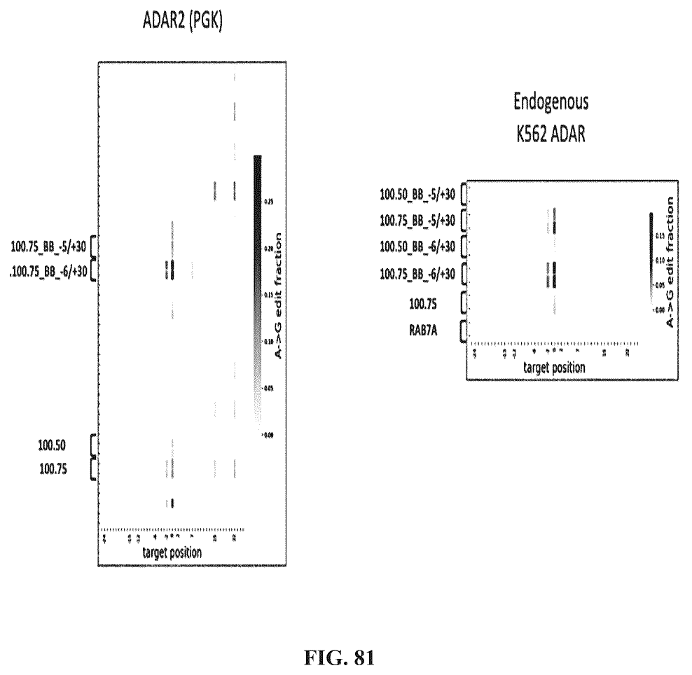

FIG. 81 depicts the editing efficiency of the exemplary guides targeting LRRK2 in the broken GFP reporter system via exogenous or endogenous ADAR.

### Example 12

### Editing Efficiency of Exemplary Circularized Engineered Guide RNAs Targeting LRRK2

HEK cells expressing endogenous ADARs and the LRRK2 minigene were utilized for these experiments. Specifically, 20,000 cells were transfected with 750 ng of plasmid and 3 μL Trans-IT 293 by reverse transfections. Cells were harvested 48 h post transfections. All linear gRNA were in expressed in a plasmid encoded U1 SmOpt format. All circular gRNA were created by flanking the antisense sequence with ribozymes, expressed from a U6 promoter in a plasmid encoded format.

TABLE 9 below contains the sequences of the engineered guide RNAs used in this example. Underlined nucleotides in the circular gRNA denote the ribozyme, ligation stem and golden gate scar.

TABLE 9
Engineered Linear and Circularized LRRK2 Guide RNA
Sequences utilized in Example 12

| SEQ | | | |
|---|---|---|---|
| ID | | | |
| NO | FIG. | Guide Name | Sequence |
| 414 | 82 | Linear 871_98.55 | CUGGUGUGCCCUCUGAUCCAAGCAUCCCCAUUCUACAG |
| | | | UAGGACUGAGCACUGCCGAGCUGGGCAAUCUUGAGUC |
| | | | GGAUGGCAGCAUUGGGAUACAGU |
| 415 | 82 | Circular 871_98.55 | GGCCAUCAGUCGCCGGUCCCAAGCCCGGAUAAAAUGG |
| | | | GAGGGGGCGGGAAACCGCCUAACCAUGCCGACUGAUG |
| | | | GCAGAUAAUCUGGUGUGCCCUCUGAUCCAAGCAUCCC |
| | | | CAUUCUACAGUAGGACUGAGCACUGCCGAGCUGGGCA |
| | | | AUCUUGAGUCGGAUGGCAGCAUUGGGAUACAGUAUAU |
| | | | ACUGCCAUCAGUCGGCGUGGACUGUAGAACACUGCCA |
| | | | AUGCCGGUCCCAAGCCCGGAUAAAAGUGGAGGGUACA |
| | | | GUCCACGCUUUUUUU |
| 416 | 82 | Linear 919_96.50 | GUGCCCUCUGAUGUUUUUAUCCGGCGAGUACAGCAGU |
| | | | UCGGAGGAAUCCCGAGGUCAGCAAUCCUAAGUAUGAU |
| | | | GGCAGCAUUGGGAUACAGUGUG |
| 417 | 82 | Circular 919_96.50 | GGCCAUCAGUCGCCGGUCCCAAGCCCGGAUAAAAUGG |
| | | | GAGGGGGGGGGAAACCGCCUAACCAUGCCGACUGAUG |
| | | | GCAGAUAAUGUGCCCUCUGAUGUUUUUAUCCGGCGAG |
| | | | UACAGCAGUUCGGAGGAAUCCCGAGGUCAGCAAUCCU |
| | | | AAGUAUGAUGGCAGCAUUGGGAUACAGUGUGAUAUAC |
| | | | UGCCAUCAGUCGGCGUGGACUGUAGAACACUGCCAAU |
| | | | GCCGGUCCCAAGCCCGGAUAAAAGUGGAGGGUACAGU |
| | | | CCACGCUUUUUUU |
| 418 | 83B | Circular 871_198.105 | GGCCAUCAGUCGCCGGUCCCAAGCCCGGAUAAAAUGG |
| | | | GAGGGGGGGGGAAACCGCCUAACCAUGCCGACUGAUG |
| | | | GCAGAUAAUCUGUUGGUUAUAAAUGACAUUUCCUCUG |
| | | | GCAACUUCAGGUGCACGAAACCCUGGUGUGCCCUCUG |
| | | | AUCCAAGCAUCCCCAUUCUACAGUAGGACUGAGCACU |
| | | | GCCGAGCUGGGCAAUCUUGAGUCGGAUGGCAGCAUUG |
| | | | GGAUACAGUGUGAAAAGCAGCACAUUGUGGGGUUUCA |
| | | | GGUAUCGGUAUAUAAUCAUGGCAUAUACUGCCAUCAG |
| | | | UCGGCGUGGACUGUAGAACACUGCCAAUGCCGGUCCC |
| | | | AAGCCCGGAUAAAAGUGGAGGGUACAGUCCACGCUUU |
| | | | UUUU |
| 419 | 83B | Circular 871_198.105_4 | GGCCAUCAGUCGCCGGUCCCAAGCCCGGAUAAAAUGG |
| | | | GAGGGGGCGGGAAACCGCCUAACCAUGCCGACUGAUG |
| | | | GCAGAUAAUCUGUUGGUUAUAAAUGACAUUUCCUGAC |
| | | | CGUACUUCAGGUGCACGAUUGGGAGGUGUGCCCUCUG |
| | | | AUCCAAGCAUCCCCAUUCUACAGUAGGACUGAGCACU |
| | | | GCCGAGCUGGGCAAUCUUGAGUCGGAUGGCAGCAUUG |
| | | | GGUAUGUCUGUGAAAAGCAGCACUAACACGGGUUUCA |
| | | | GGUAUCGGUAUAUAAUCAUGGCAUAUACUGCCAUCAG |
| | | | UCGGCGUGGACUGUAGAACACUGCCAAUGCCGGUCCC |
| | | | AAGCCCGGAUAAAAGUGGAGGGUACAGUCCACGCUUU |
| | | | UUUU |
| 420 | 83B | Circular 919_196.100 | GGCCAUCAGUCGCCGGUCCCAAGCCCGGAUAAAAUGG |
| | | | GAGGGGGCGGGAAACCGCCUAACCAUGCCGACUGAUG |
| | | | GCAGAUAAUGGUUAUAAAUGACAUUUCCUCUGGCAAC |
| | | | UUCAGGUGCACGAAACCCUGGUGUGCCCUCUGAUGUU |
| | | | GUUAUCCGGCGAGUACAGCAGUUCGGAGGAAUCCCGA |
| | | | GGUCAGCAAUCCUAAGUAUGAUGGCAGCAUUGGGAUA |
| | | | CAGUGUGAAAAGCAGCACAUUGUGGGGUUUCAGGUAU |
| | | | CGGUAUAUAAUCAUGGCUGAAUAUACUGCCAUCAGUC |
| | | | GCCGUGGACUGUAGAACACUGCCAAUGCCGGUCCCAA |
| | | | GCCCGGAUAAAAGUGGAGGGUACAGUCCACGCUUUUU |
| | | | UU |
| 421 | 83B | Circular 919_196.100_4 | GGCCAUCAGUCGCCGGUCCCAAGCCCGGAUAAAAUGG |
| | | | GAGGGGGGGGGAAACCGCCUAACCAUGCCGACUGAUG |
| | | | GCAGAUAAUGGUUAUAAAUGACAUUUCCUCUGGCAAC |
| | | | UUGUCCACCACGAAACCCUGGUGACGGGACUGAUGUU |
| | | | GUUAUCCGGCGAGUACAGCAGUUCGGAGGAAUCCCGA |
| | | | GGUCAGCAAUCCUAAGUAUGAUGGCAGCAUUGCCUAU |
| | | | GAGUGUGAAAAGCAGCUGUAACUGGGGUUUCAGGUAU |
| | | | CGGUAUAUAAUCAUGGCUGAAUAUACUGCCAUCAGUC |
| | | | GGCGUGGACUGUAGAACACUGCCAAUGCCGGUCCCAA |
| | | | GCCCGGAUAAAAGUGGAGGGUACAGUCCACGCUUUUU |
| | | | UU |
| 422 | 83A | Circular 871_128.70 | GGCCAUCAGUCGCCGGUCCCAAGCCCGGAUAAAAUGG |
| | | | GAGGGGGCGGGAAACCGCCUAACCAUGCCGACUGAUG |
| | | | GCAGAUAAUCAGGUGCACGAAACCCUGGUGUGCCCUC |
| | | | UGAUCCAAGCAUCCCCAUUCUACAGUAGGACUGAGCA |
| | | | CUGCCGAGCUGGGCAAUCUUGAGUCGGAUGGCAGCAU |
| | | | UGGGAUACAGUGUGAAAAGCAGCACAAUAUACUGCCA |
| | | | UCAGUCGGCGUGGACUGUAGAACACUGCCAAUGCCGG |
| | | | UCCCAAGCCCGGAUAAAAGUGGAGGGUACAGUCCACG |
| | | | CUUUUUUU |
| 423 | 83A | Circular 871_158.85 | GGCCAUCAGUCGCCGGUCCCAAGCCCGGAUAAAAUGG |
| | | | GAGGGGGCGGGAAACCGCCUAACCAUGCCGACUGAUG |
| | | | GCAGAUAAUUUCCUCUGGCAACUUCAGGUGCACGAAA |
| | | | CCCUGGUGUGCCCUCUGAUCCAAGCAUCCCCAUUCUAC |
| | | | AGUAGGACUGAGCACUGCCGAGCUGGGCAAUCUUGAG |
| | | | UCGGAUGGCAGCAUUGGGAUACAGUGUGAAAAGCAGC |
| | | | ACAUUGUGGGGUUUCAGGAUAUACUGCCAUCAGUCGG |
| | | | CGUGGACUGUAGAACACUGCCAAUGCCGGUCCCAAGC |
| | | | CCGGAUAAAAGUGGAGGGUACAGUCCACGCUUUUUUU |
| 424 | 83A | Circular 919_126.65 | GGCCAUCAGUCGCCGGUCCCAAGCCCGGAUAAAAUGG |
| | | | GAGGGGGCGGGAAACCGCCUAACCAUGCCGACUGAUG |
| | | | GCAGAUAAUGCACGAAACCCUGGUGUGCCCUCUGAUG |
| | | | UUGUUAUCCGGCGAGUACAGCAGUUCGGAGGAAUCCC |
| | | | GAGGUCAGCAAUCCUAAGUAUGAUGGCAGCAUUGGGA |
| | | | UACAGUGUGAAAAGCAGCACAUUGAUAUACUGCCAUC |
| | | | AGUCGGCGUGGACUGUAGAACACUGCCAAUGCCGGUC |
| | | | CCAAGCCCGGAUAAAAGUGGAGGGUACAGUCCACGCU |
| | | | UUUUUU |
| 425 | 83A | Circular 919_156.80 | GGCCAUCAGUCGCCGGUCCCAAGCCCGGAUAAAAUGG |
| | | | GAGGGGGCGGGAAACCGCCUAACCAUGCCGACUGAUG |
| | | | GCAGAUAAUCUGGCAACUUCAGGUGCACGAAACCCUG |
| | | | GUGUGCCCUCUGAUGUUGUUAUCCGGCGAGUACAGCA |
| | | | GUUCGGAGGAAUCCCGAGGUCAGCAAUCCUAAGUAUG |
| | | | AUGGCAGCAUUGGGAUACAGUGUGAAAAGCAGCACAU |
| | | | UGUGGGGUUUCAGGUCUAUAUACUGCCAUCAGUCGGC |
| | | | GUGGACUGUAGAACACUGCCAAUGCCGGUCCCAAGCC |
| | | | CGGAUAAAAGUGGAGGGUACAGUCCACGCUUUUUUU |
| 426 | 84 | Circular 919_196.100_2 | GGCCAUCAGUCGCCGGUCCCAAGCCCGGAUAAAAUGG |
| | | | GAGGGGGCGGGAAACCGCCUAACCAUGCCGACUGAUG |
| | | | GCAGAUAAUGGUUAUAAAUGACAUUUCCUCUGGCAAC |
| | | | UUCAGGUGCACGAAACCCUGGUGACGGGACUGAUGUU |
| | | | GUUAUCCGGCGAGUACAGCAGUUCGGAGGAAUCCCGA |
| | | | GGUCAGCAAUCCUAAGUAUGAUGGCAGCAUUGCCUAU |
| | | | GAGUGUGAAAAGCAGCACAUUGUGGGGUUUCAGGUAU |
| | | | CGGUAUAUAAUCAUGGCUGAAUAUACUGCCAUCAGUC |
| | | | GGCGUGGACUGUAGAACACUGCCAAUGCCGGUCCCAA |
| | | | GCCCGGAUAAAAGUGGAGGGUACAGUCCACGCUUUUU |
| | | | UU |
| 427 | 84 | Circular | GGCCAUCAGUCGCCGGUCCCAAGCCCGGAUAAAAUGG |
| | | 919_196.100_2_U- | GAGGGGGGCGGAAACCGCCUAACCAUGCCGACUGAUG |
| | | deletions | GCAGAUAAUGGUUAUAAAUGACAUUUCCUCUGGCAAC |
| | | | CAGGUGCACGAAACCCUGGUGACGGGACUGAUGUUGU |
| | | | UAUCCGGCGAGUACAGCAGUUCGGAGGAAUCCCGAGG |
| | | | UCAGCAAUCCUAAGUAUGAUGGCAGCAUUGCCUAUGA |
| | | | GUGUGAAAAGCAGCACAUUGUGGGGCAGGAUCGGAUA |
| | | | AAUCAUGGCUGAAUAUACUGCCAUCAGUCGGCGUGGA |
| | | | CUGUAGAACACUGCCAAUGCCGGUCCCAAGCCCGGAU |
| | | | AAAAGUGGAGGGUACAGUCCACGCUUUUUUU |

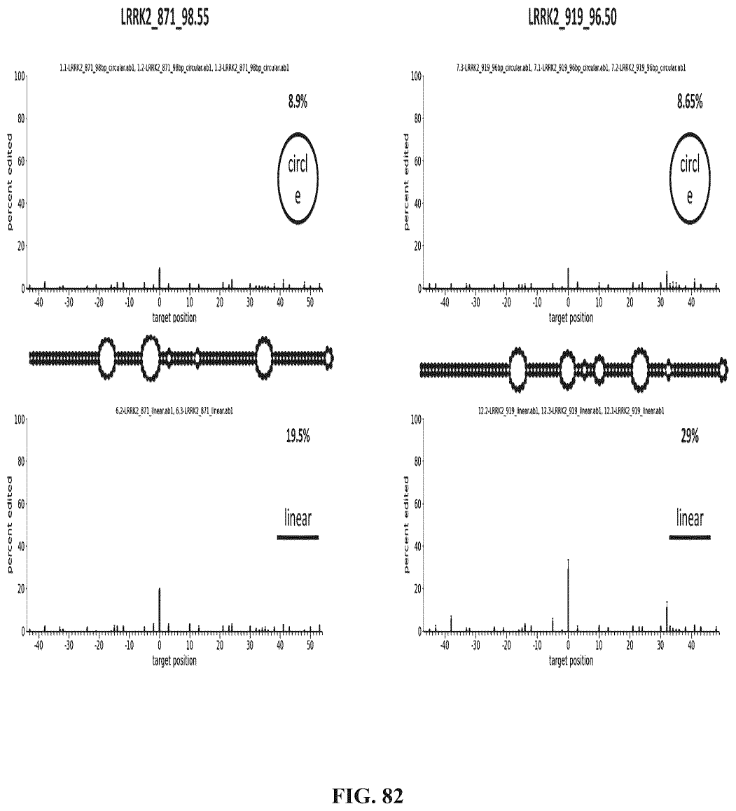

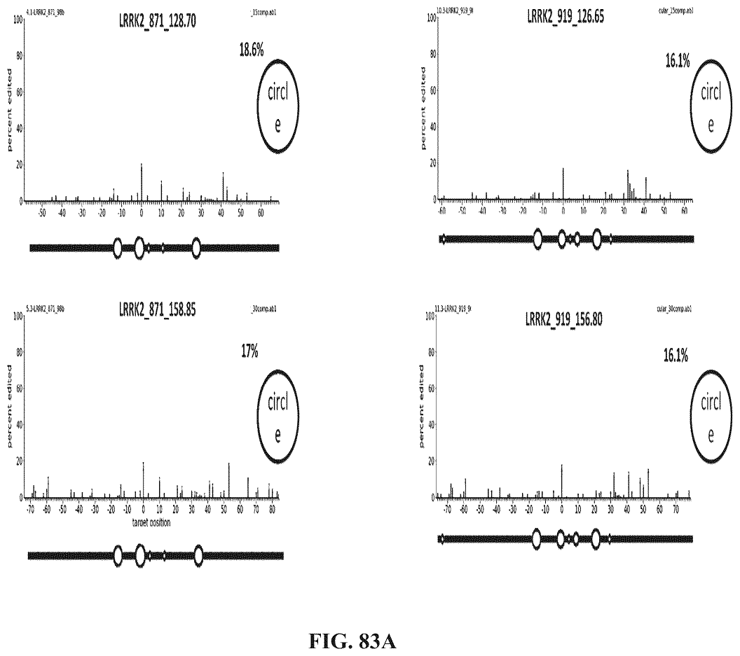

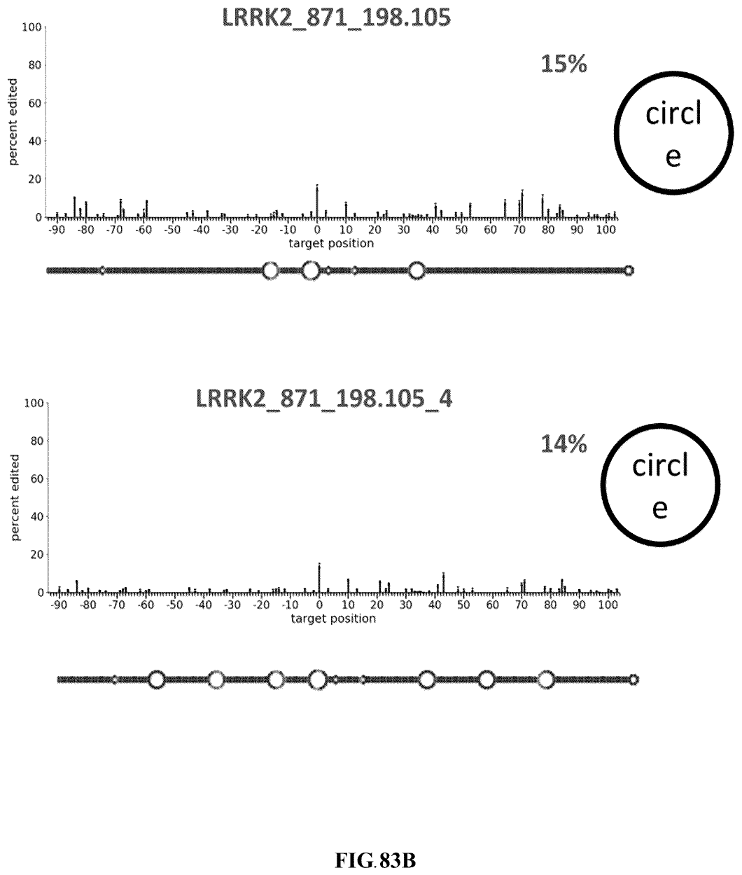

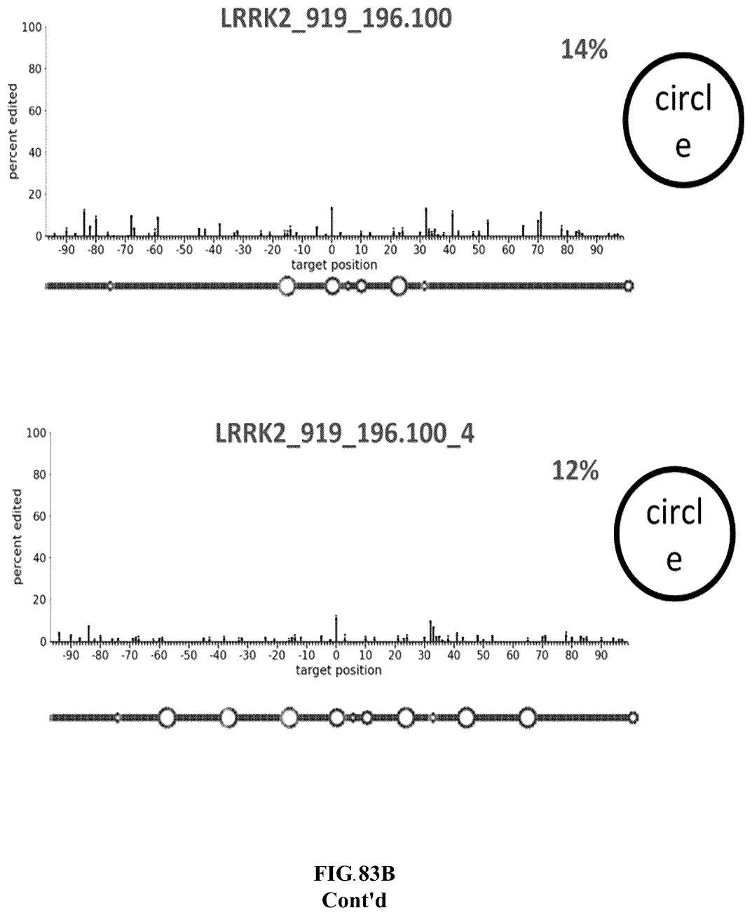

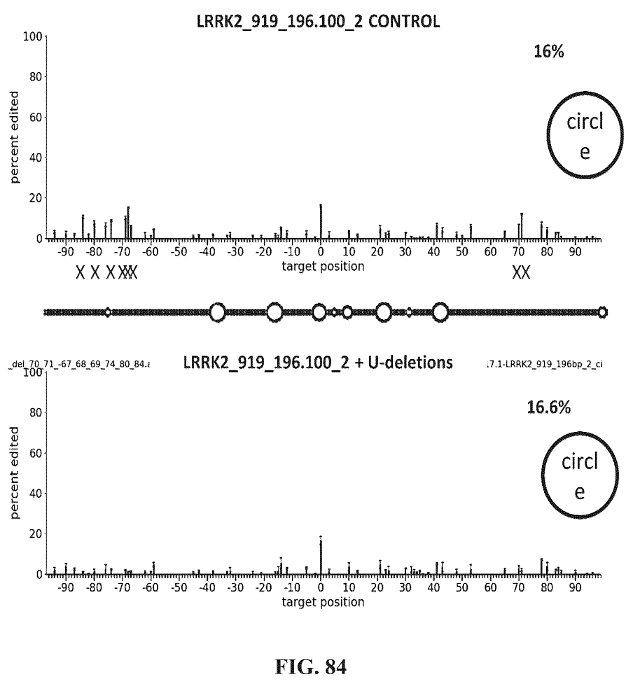

FIG. 82 provides a comparison between linear and circularized versions of exemplary guide RNAs guide871 and guide919 targeting LRRK2. While the editing efficiency of the circularized versions of the guide RNAs were lower than the linear counterparts, editing efficiency was increased by lengthening the circularized guide RNAs by an additional 15 nucleotides (FIG. 83A), 30 nucleotides (FIG. 83A), and 100 nucleotides (FIG. 83B). Finally, selected uridines were deleted from the circularized guide 919 to produce a U-deletion variant, and the effect of the U deletions on editing is depicted in FIG. 84.

### Example 13

### In Vivo Efficacy of AAV Vector Encoding an Engineered Guide RNA Targeting LRRK2

The scAAV vector constructed in EXAMPLE 9 was utilized in this example. Following QC validation of on-target LRRK2 G2019S editing and guide expression, the scAAV vector was packaged into an scAAVDJ virus for in vivo testing in LRRK2 G2019S transgenic mice.

### Experimental Animals

Hemizygous BAC LRRK2 G2019S transgenic mice were utilized for this example (C57BL/U6J-Tg(LRRK2*G2019S)2AMjff/J; Strain #018785).

### Thy1.1 Enrichment

Brain (ICV) and liver (IV) tissue samples were dissected from experimental mice and dissociated into single-cell suspensions using gentleMACS Dissociator (Miltenyi Biotec). Following dissociation, an aliquot of each sample was set aside and designated as “Pre-Enrichment”. The remainder of the samples were enriched for Thy1.1-expressing cells using CD90.1 MicroBeads (Miltenyi Biotec; Cat #130-121-273) by MACS and designated as “Post-Enrichment”.

### Sanger Sequencing/EditADAR Analysis

RNA extraction of “Pre-enrichment” and “Post-enrichment” brain and liver samples was performed using mirVana™ miRNA isolation kit (ThermoFisher) per manufacturer protocol. Synthesis of cDNA was performed using ProtoScript® II First Strand cDNA synthesis kit (NEB) per manufacturer protocol. PCR amplification of the LRRK2 G2019S target locus was performed using Q5® High-Fidelity 2× master mix (NEB) using the following specific primers and thermocycler settings:

| Primer | Sequence |
|---|---|
| hLRRK2_6020F | acaaagccagcctcactaga (SEQ ID NO: 428) |
| hLRRK2_6524R | tcaaagacctgggcagaagt (SEQ ID NO: 429) |
| Thermocycler Settings | 98° C.-30s |
| | [35 cycles]98° C.-10s, 67º C.-30s, 72° C.-20s |
| | 72° C.-2 min |
| | 4º C.-hold |

Following PCR amplification and gel confirmation of specific product, ExoSAP-IT™ PCR product cleanup reagent (ThermoFisher) was added to samples prior to Sanger sequencing submission. Sequencing trace files (.abl) were analyzed via EditADAR (ShapeTX) to generate RNA editing profiles.

ddPCR Guide RNA Quantitation

cDNA synthesis of “Pre-enrichment” and “Post-enrichment” brain and liver RNA samples was performed using ProtoScript® II First Strand cDNA synthesis kit (NEB) per manufacturer protocol with the following modification—use of smOPT specific primer (5′-CAGAAAACCTGCTCCAAAAATTCCAC-3′) with oligo d(T)23 VN at 1:1 ratio. ddPCR was performed on the QX200 system (Bio-Rad) using ddPCR Supermix for Probes (No dUTP) (Bio-Rad; Cat #1863024) using the following specific ddPCR primers and probes and thermocycler settings:

| Primer/Probe | Sequence |
|---|---|
| (F) | GTGTGCCCTCTGATGTTTTTC (SEQ ID NO: 430) |
| (R) | CCCAATGCTTCGCTTATTGC (SEQ ID NO: 431) |
| (Probe) | /56FAM/ACAACAGTA/ZEN/CTGAGCTATCCCG/3IABKFQ/ |
| | (SEQ ID NO: 432) |
| GAPDH (F) | TGTAGTTGAGGTCAATGAAGGG (SEQ ID NO: 433) |
| GAPDH (R) | ACATCGCTCAGACACCATG (SEQ ID NO: 434) |
| GAPDH (Probe) | /5HEX/AAGGTCGGA/ZEN/GTCAACGGATTTGGTC/3IABKFQ/ |
| | (SEQ ID NO: 435) |
| Thermocycler Settings | 95° C.-10min |
| | [40 cycles]94° C.-30s(2° C./s), 60° C.-60s(2° C./s), |
| | 72° C.-15s(2° C./s) |
| | 98° C.-10 min |
| | 98° C.-10 min |
| | 4° C.- hold |

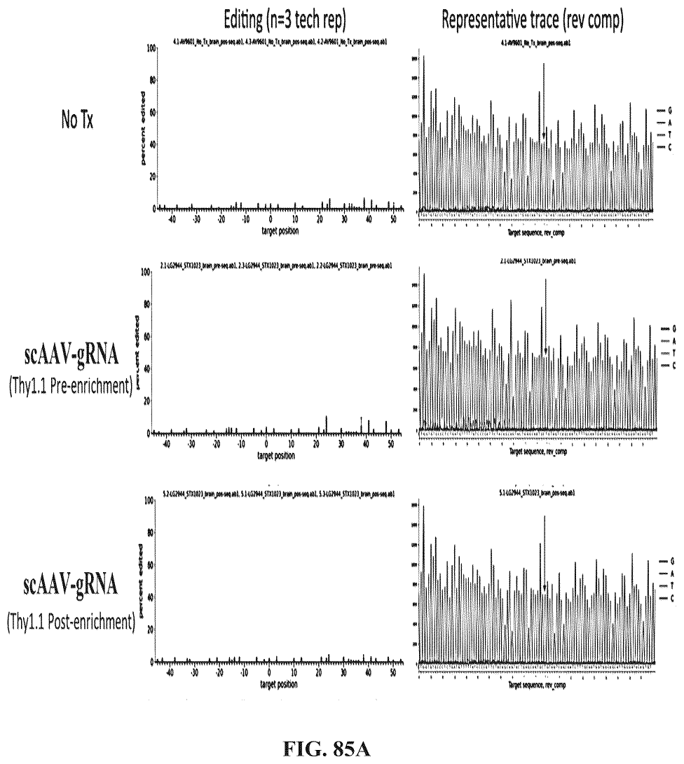

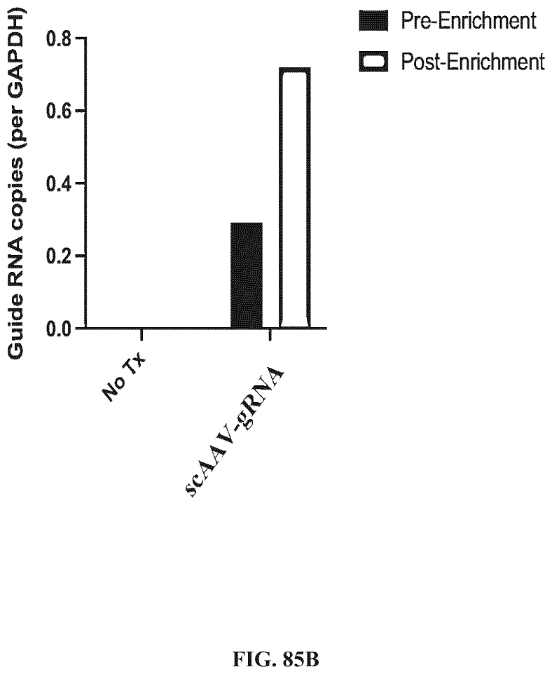

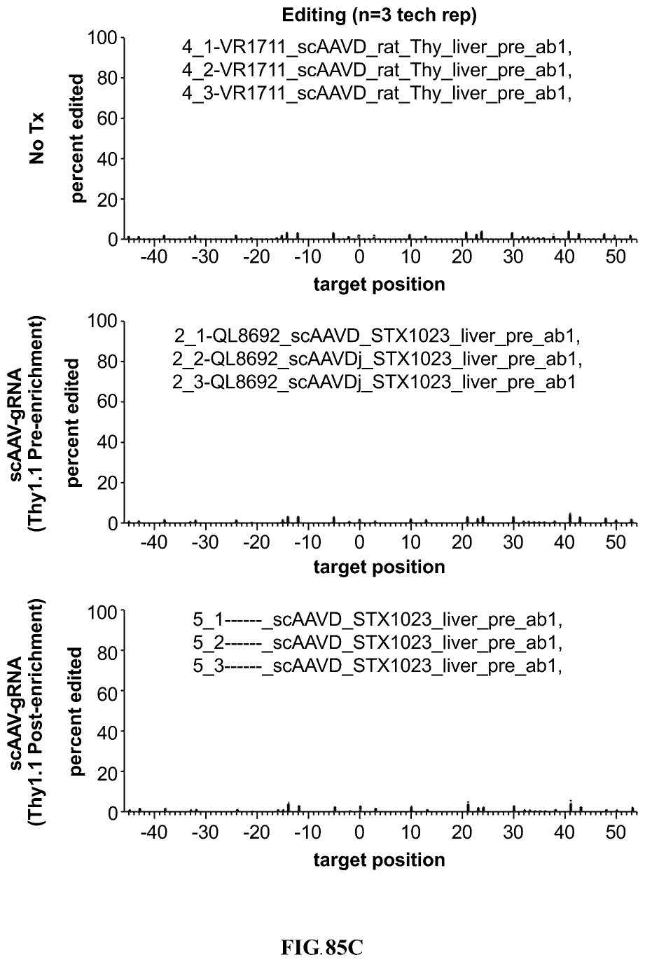

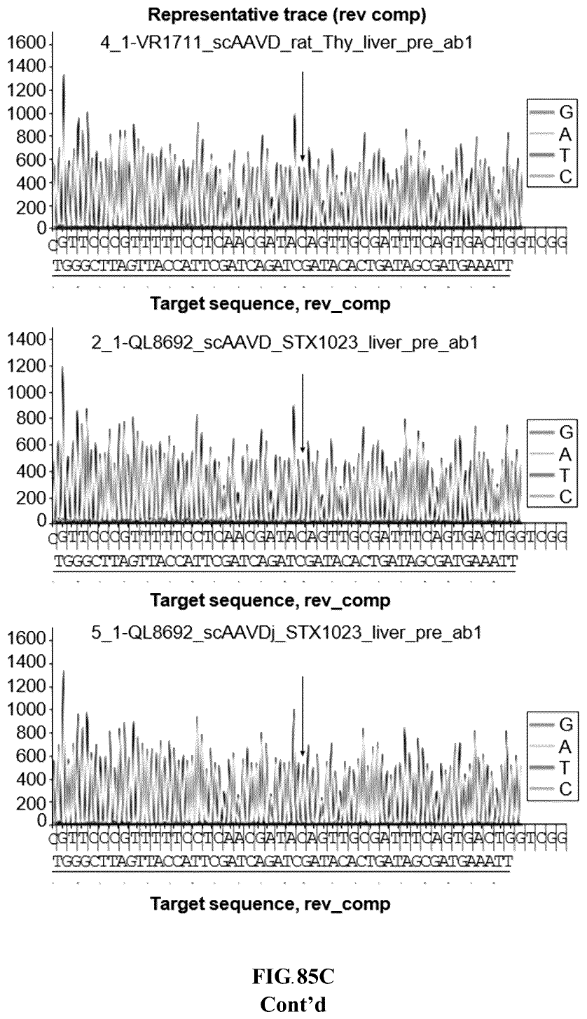

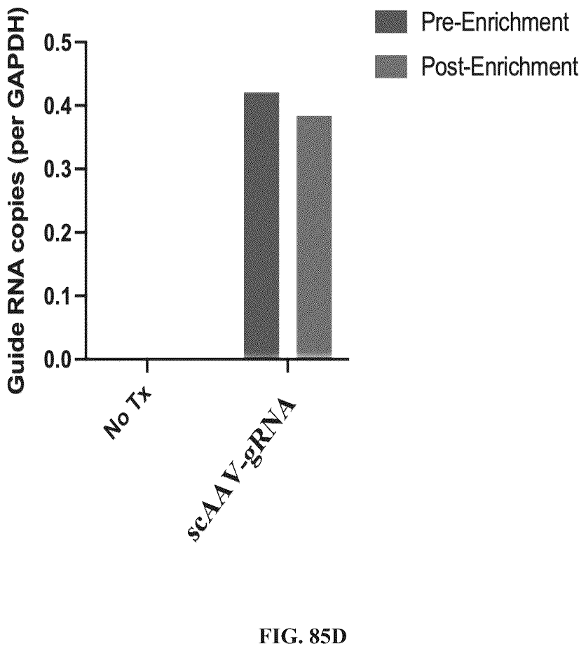

Absolute ddPCR values for the engineered guide RNA and GAPDH were recorded as copies/uL. Guide RNA copy numbers were normalized to GAPDH copy numbers. FIG. 85A and FIG. 85C depict the in vivo editing efficiencies for the scAAV vector encoding the engineered guide RNA targeting LRRK2, as measured in the brain (FIG. 85A) and liver (FIG. 85C). No detectable editing observed in the scAAVDJ-engineered guide RNA ICV-injected mouse brain or liver tissue. EditADAR analysis and representative Sanger sequencing traces from no treatment, Thy1.1 pre-enrichment scAAVDJ-engineered guide RNA and Thy1.1 post-enrichment scAAVDJ-engineered guide RNA brain RNA samples. FIG. 85B and FIG. 85D illustrate quantitation of engineered guide RNA expression, as compared to expression of the GAPDH control, in the brain (FIG. 85B) and liver (FIG. 85D). Low levels of guide RNA expression (<1 guide RNA copy per GAPDH) in both Thy1.1 pre- and post-enrichment brain and liver samples as measured by ddPCR analysis was detected.

While preferred embodiments of the present disclosure have been shown and described herein, such embodiments are provided by way of example only. Numerous variations, changes, and substitutions can occur without departing from the present disclosure. It should be understood that various alternatives to the embodiments described herein may be employed.

What is claimed is:

1.-33. (canceled)

34. An engineered guide RNA comprising a targeting sequence with complementarity to a sequence of a target LRRK2 RNA, wherein the complementarity is sufficient for the engineered guide RNA to hybridize to the target LRRK2 RNA, thereby forming a guide-target RNA scaffold that is a substrate for an adenosine deaminase acting on RNA (ADAR) enzyme, wherein formation of the guide-target RNA scaffold substantially forms a micro-footprint and a barbell macro-footprint that each independently comprise structural features that are not present in the engineered guide RNA prior to the formation of the guide-target RNA scaffold; wherein
(a) the structural features of the micro-footprint comprise:
(i) a mismatch formed between the sequence of the target LRRK2 RNA and the engineered guide RNA, and
(ii) at least one additional structural feature selected from the group consisting of: a bulge, a hairpin, an internal loop, a wobble base pair, and any combination thereof, and
(b) the structural features of the barbell macro-footprint comprise:
(i) a first internal loop that is 5′ of the micro-footprint; and
(ii) a second internal loop that is 3′ of the micro-footprint; and,
wherein the guide-target RNA scaffold that comprises the micro-footprint and the barbell macro-footprint, upon contact with the ADAR enzyme, facilitates an increase in the amount of editing of an on-target adenosine in the sequence of the target LRRK2 RNA by the ADAR enzyme, relative to an otherwise comparable engineered guide RNA that upon hybridizing to the target LRRK2 RNA results in formation of a guide-target RNA scaffold lacking the micro-footprint and the barbell macro-footprint.

35. The engineered guide RNA of claim 34, wherein the engineered guide RNA comprises at least 80%, at least 85%, at least 90%, at least 92%, at least 95%, at least 99%, or 100% sequence identity to any one of SEQ ID NO: 2-SEQ ID NO: 395 or SEQ ID NO: 398-SEQ ID NO: 427.

36. The engineered guide RNA of claim 34, wherein the engineered guide RNA comprises at least 20-50 contiguous nucleotides from any one of SEQ ID NO: 2-SEQ ID NO: 395 or SEQ ID NO: 398-SEQ ID NO: 427.

37. The engineered guide RNA of claim 34, wherein the mismatch of the micro-footprint is an A/C mismatch formed between the on-target adenosine in the target LRRK2 RNA and the cytosine in the engineered guide RNA.

38. The engineered guide RNA of claim 37, wherein the first internal loop that is 5′ of the micro-footprint is positioned from 7 bases away from the A/C mismatch to about 30 bases away from the A/C mismatch, with respect to the base of the first internal loop that is most proximal to the A/C mismatch.

39. The engineered guide RNA of claim 37, wherein the second internal loop that is 3′ of the micro-footprint is positioned from 18 bases away from the A/C mismatch to 34 bases away from the A/C mismatch, with respect to the base of the second internal loop that is most proximal to the A/C mismatch.

40. The engineered guide RNA of claim 34, wherein the at least one additional structural feature of the micro-footprint comprises the bulge, wherein the bulge comprises a symmetric bulge or an asymmetric bulge.

41. The engineered guide RNA of claim 34, wherein the at least one additional structural feature of the micro-footprint comprises the internal loop, wherein the internal loop is a symmetric internal loop or an asymmetric internal loop.

42. The engineered guide RNA of claim 34, wherein the at least one additional structural feature of the micro-footprint comprises the hairpin, wherein the hairpin is a recruitment hairpin or a non-recruitment hairpin.

43. The engineered guide RNA of claim 34, wherein the target LRRK2 RNA encodes a LRRK2 polypeptide having a mutation with respect to a wild-type LRRK2 polypeptide, wherein the mutation is selected from the group consisting of: E10L, A30P, 552F, E46K, A53T, L119P, A211V, C228S, E334K, N363S, V366M, A419V, R506Q, N544E, N551K, A716V, M712V, 1723V, P755L, R793M, 1810V, K871E, Q923H, Q930R, R1067Q, S1096C, Q1111H, I1122V, A1151T, L1165P, I1192V, H1216R, S1228T, P1262A, R1325Q, I1371V, R1398H, T1410M, D1420N, R1441G, R1441H, A1442P, P1446L, V1450I, K1468E, R1483Q, R1514Q, P1542S, V1613A, R1628P, M1646T, S1647T, Y1699C, R1728H, R1728L, L1795F, M1869V, M1869T, L1870F, E1874X, R1941H, Y2006H, I2012T, G2019S, I2020T, T2031S, N2081D, T2141M, R2143H, Y2189C, T2356I, G2385R, V2390M, E2395K, M2397T, L2466H, and Q2490NfsX3.

44. The engineered guide RNA of claim 34, wherein the ADAR enzyme comprises ADAR1, ADAR2, ADAR3, or any combination thereof.

45. A vector comprising a polynucleotide encoding an engineered guide RNA comprising a targeting sequence with complementarity to a sequence of a target LRRK2 RNA, wherein the complementarity is sufficient for the engineered guide RNA to hybridize to the target LRRK2 RNA, thereby forming a guide-target RNA scaffold that is a substrate for an adenosine deaminase acting on RNA (ADAR) enzyme, wherein formation of the guide-target RNA scaffold substantially forms a micro-footprint and a barbell macro-footprint that each independently comprise structural features that are not present in the engineered guide RNA prior to the formation of the guide-target scaffold; wherein
a. the structural features of the micro-footprint comprise:
i. a mismatch formed between the sequence of the target LRRK2 RNA and the engineered guide RNA, and
ii. at least one additional structural feature selected from the group consisting of: a bulge, an internal loop, a wobble base pair, and any combination thereof; and
b. the structural features of the barbell macro-footprint comprise:
i. a first internal loop that is 5′ of the micro-footprint; and
ii. a second internal loop that is 3′ of the micro-footprint;
and wherein the guide-target RNA scaffold that comprises the micro-footprint and the barbell macro-footprint, upon contact with the ADAR enzyme, facilitates an increase in the amount of editing of an on-target adenosine in the sequence of the target LRRK2 RNA by the ADAR enzyme, relative to an otherwise comparable engineered guide RNA that upon hybridizing to the target LRRK2 RNA results in formation of a guide-target RNA scaffold lacking the micro-footprint and the barbell macro-footprint.

46. The vector of claim 45, wherein the vector is a viral vector, and wherein the polynucleotide encoding the engineered guide RNA is encapsidated in the viral vector.

47. The vector of claim 45, wherein the viral vector is an adeno-associated viral (AAV) vector or a derivative thereof.

48. The vector of claim 47, wherein the AAV vector is AAV1, AAV2, AAV3, AAV4, AAV5, AAV6, AAV7, AAV8, AAV9, AAV10, AAV11, or a derivative, a chimera, or a variant thereof.

49. The vector of claim 48, wherein the AAV vector is a recombinant AAV (rAAV) vector, a hybrid AAV vector, a chimeric AAV vector, a self-complementary AAV (scAAV) vector, or any combination thereof.

50. A pharmaceutical composition in unit dose form that comprises:
(a) the vector of claim 45, and
(b) a pharmaceutically acceptable: excipient, diluent, or carrier.

51. A method of treating a disease in a subject in need thereof, the method comprising administering to the subject an effective amount of an engineered guide RNA or a polynucleotide encoding the engineered guide RNA, wherein the engineered guide RNA comprises a sequence with complementarity to a sequence of a target LRRK2 RNA, wherein the complementarity is sufficient for the engineered guide RNA to hybridize to the target LRRK2 RNA, thereby forming a guide-target RNA scaffold that is a substrate for an adenosine deaminase acting on RNA (ADAR) enzyme, wherein the guide-target RNA scaffold comprises a micro-footprint and a barbell macro-footprint; wherein:
(a) the micro-footprint comprises at least one structural feature formed upon hybridization of the sequence of the engineered guide RNA to the sequence of the target LRRK2 RNA that is selected from the group consisting of: a bulge, an internal loop, a mismatch, a wobble base pair, and any combination thereof; and
(b) the barbell macro-footprint comprises a first internal loop and a second internal loop that are each formed upon hybridization of the sequence of the engineered guide RNA to the sequence of the target LRRK2 RNA, wherein the first internal loop is 5′ of the micro-footprint and the second internal loop is 3′ of the micro-footprint; and
wherein the disease is selected from the group consisting of: Parkinson's disease, Crohn's disease, and any combination thereof.

52. The method of claim 51, wherein the subject has a mutation in an LRRK2 polypeptide with respect to a wildtype LRRK2 polypeptide, wherein the mutation is selected from the group consisting of: E10L, A30P, S52F, E46K, A53T, L119P, A211V, C228S, E334K, N363S, V366M, A419V, R506Q, N544E, N551K, A716V, M712V, I723V, P755L, R793M, I810V, K871E, Q923H, Q930R, R1067Q, S1096C, Q1111H, I1122V, A1151T, L1165P, I1192V, H1216R, S1228T, P1262A, R1325Q, I1371V, R1398H, T1410M, D1420N, R1441G, R1441H, A1442P, P1446L, V1450I, K1468E, R1483Q, R1514Q, P1542S, V1613A, R1628P, M1646T, S1647T, Y1699C, R1728H, R1728L, L1795F, M1869V, M1869T, L1870F, E1874X, R1941H, Y2006H, I2012T, G2019S, I2020T, T2031S, N2081D, T2141M, R2143H, Y2189C, T2356I, G2385R, V2390M, E2395K, M2397T, L2466H, Q2490NfsX3, and any combination thereof.

53. The method of claim 52, wherein the mutation in the LRRK2 polypeptide is G2019S.

54. The method of claim 51, wherein the subject is human or a non-human animal.


SEQUENCE LISTING (441 sequences)

SEQ ID NO: 1 - RNA, 45 nt, unidentified
  source 1..45: mol_type = unassigned RNA
gtggaatagtataacaatatgctaaatgttgttatagtatcccac

SEQ ID NO: 2 - DNA, 113 bp, synthetic construct
  source 1..113: mol_type = other DNA
ccctggtgtgccctctgatgtttttatccccattctacagcggtactgagcaaatccgtggtcagcaatctttgcaatgatggcagcattgggatacagtgtgaagagcagca

SEQ ID NO: 3 - DNA, 113 bp, synthetic construct
  source 1..113: mol_type = other DNA
ccctggtgtgccctctgatgttttttaggggattctacagcggtactgagcaaatccgtggtcagcaatcaaacgtatgatggcagcattgggatacagtgtgaagagcagca

SEQ ID NO: 4 - DNA, 113 bp, synthetic construct
  source 1..113: mol_type = other DNA
ccctggtgtgccctctgatgtttttatccccattctacatctgtagtgagcaattccgtgctcagcaatctttgcaatgatggcagcattgggatacagtgtgaagagcagca

SEQ ID NO: 5 - DNA, 113 bp, synthetic construct
  source 1..113: mol_type = other DNA
ccctggtgtgccctctgatgttttttaggggattctacatctgtagtgagcaattccgtgctcagcaatcaaacgtatgatggcagcattgggatacagtgtgaagagcagca

SEQ ID NO: 6 - DNA, 113 bp, synthetic construct
  source 1..113: mol_type = other DNA
ccctggtgtgccctctgatgtttttatccccattctacacctggactgagaaatcccgtactcagcaatctttgcaatgatggcagcattgggatacagtgtgaagagcagca

SEQ ID NO: 7 - DNA, 113 bp, synthetic construct
  source 1..113: mol_type = other DNA
ccctggtgtgccctctgatgttttttaggggattctacacctggactgagaaatcccgtactcagcaatcaaacgtatgatggcagcattgggatacagtgtgaagagcagca

SEQ ID NO: 8 - DNA, 113 bp, synthetic construct
  source 1..113: mol_type = other DNA
ccctggtgtgccctctgatgtttttatccccattctacagcagtacggtgcagtgccgtggtcagcaatctttgcaatgatggcagcattgggatacagtgtgaagagcagca

SEQ ID NO: 9 - DNA, 113 bp, synthetic construct
  source 1..113: mol_type = other DNA
ccctggtgtgccctctgatgttttttaggggattctacagcagtacggtgcagtgccgtggtcagcaatcaaacgtatgatggcagcattgggatacagtgtgaagagcagca

SEQ ID NO: 10 - DNA, 113 bp, synthetic construct
  source 1..113: mol_type = other DNA
ccctggtgtgccctctgatgtttttatcccccttctacagcagtagtgagttttgccgtggtcagcaatctttgcaatgatggcagcattgggatacagtgtgaagagcagca

SEQ ID NO: 11 - DNA, 113 bp, synthetic construct
  source 1..113: mol_type = other DNA
ccctggtgtgccctctgatgttttttaggggcttctacagcagtagtgagttttgccgtggtcagcaatcaaacgtatgatggcagcattgggatacagtgtgaagagcagca

SEQ ID NO: 12 - DNA, 113 bp, synthetic construct
  source 1..113: mol_type = other DNA
ccctggtgtgccctctgatgtttttatccccattctacagcagtcctgtacagtgccgtgatcagcaatctttgcaatgatggcagcattgggatacagtgtgaagagcagca

SEQ ID NO: 13 - DNA, 113 bp, synthetic construct
  source 1..113: mol_type = other DNA
ccctggtgtgccctctgatgttttttaggggattctacagcagtcctgtacagtgccgtgatcagcaatcaaacgtatgatggcagcattgggatacagtgtgaagagcagca

SEQ ID NO: 14 - DNA, 113 bp, synthetic construct
  source 1..113: mol_type = other DNA
ccctggtgtgccctctgatgtttttatccccattcgcctgaggtactgaccaatcccgtagttagcctaggctgcaatgatggcagcattgggatacagtgtgaagagcagca

SEQ ID NO: 15 - DNA, 113 bp, synthetic construct
  source 1..113: mol_type = other DNA
ccctggtgtgccctctgatgtttttctacagattctacagcagtacagaggactgccgaggtcaccaatctttgcaataacctgagcattgggatacagtgtgaagagcagca

SEQ ID NO: 16 - DNA, 113 bp, synthetic construct
  source 1..113: mol_type = other DNA
ccctggtgtgccctctgatgttttttaggggattctacagcagtacagaggactgccgaggtcaccaatctttgcatactacgcagcattgggatacagtgtgaagagcagca

SEQ ID NO: 17 - DNA, 113 bp, synthetic construct
  source 1..113: mol_type = other DNA
ccctggtgtgccctctgatgtttttcgggagattctacagcagtacagaggactgccgaggtcaccaatcttgtctaggatggcagcattgggatacagtgtgaagagcagca

SEQ ID NO: 18 - DNA, 113 bp, synthetic construct
  source 1..113: mol_type = other DNA
ccctggtgtgccctctgatgtatgttcccccattctacagcagtacagaggactgccgaggtcaccaacgctaacaatgatggcagcattgggatacagtgtgaagagcagca

SEQ ID NO: 19 - DNA, 113 bp, synthetic construct
  source 1..113: mol_type = other DNA
ccctggtgtgccctctgatgtttttatggaaagtctacagcagtacagaggactgccgaggtcactttgatttgcaatgatggcagcattgggatacagtgtgaagagcagca

SEQ ID NO: 20 - DNA, 113 bp, synthetic construct
  source 1..113: mol_type = other DNA
ccctggtgtgccctctgatgtttttcgaagaattctacagtaggactgagcactgccgagctgggcaatctttgcaatcaaaaaagcattgggatacagtgtgaagagcagca

SEQ ID NO: 21 - DNA, 113 bp, synthetic construct
  source 1..113: mol_type = other DNA
ccctggtgtgccctctgatgttttttaggggattctacagtaggactgagcactgccgagctgggcaatctttgcatactacgcagcattgggatacagtgtgaagagcagca

SEQ ID NO: 22 - DNA, 113 bp, synthetic construct
  source 1..113: mol_type = other DNA
ccctggtgtgccctctgatgtttttctacagattctacagtaggactgagcactgccgagctgggcaatcttcatttggatggcagcattgggatacagtgtgaagagcagca

SEQ ID NO: 23 - DNA, 113 bp, synthetic construct
  source 1..113: mol_type = other DNA
ccctggtgtgccctctgatgtttttatggggtgtctacagtaggactgagcactgccgagctgggcaatcttgagtcggatggcagcattgggatacagtgtgaagagcagca

SEQ ID NO: 24 - DNA, 113 bp, synthetic construct
  source 1..113: mol_type = other DNA
ccctggtgtgccctctgatgtttttatccgaaacatacagtaggactgagcactgccgagctgggcaatcttctcccagatggcagcattgggatacagtgtgaagagcagca

SEQ ID NO: 25 - DNA, 113 bp, synthetic construct
  source 1..113: mol_type = other DNA
ccctggtgtgccctctgatgtttttcatgagattctacagtaggactgagcactgccgagctgggcctcaggtgcaatgatggcagcattgggatacagtgtgaagagcagca

SEQ ID NO: 26 - DNA, 113 bp, synthetic construct
  source 1..113: mol_type = other DNA
ccctggtgtgccctctgatgttttttacagaattctacagtaggactgagcactgccgagctggggttcctttgcaatgatggcagcattgggatacagtgtgaagagcagca

SEQ ID NO: 27 - DNA, 113 bp, synthetic construct
  source 1..113: mol_type = other DNA
ccctggtgtgccctctgatgttttttaggggattctaccgcagtactacccgatcccgtagtcagcaatctttgcatactacgcagcattgggatacagtgtgaagagcagca

SEQ ID NO: 28 - DNA, 113 bp, synthetic construct
  source 1..113: mol_type = other DNA
ccctggtgtgccctctgatgtttttatccccattctagggcagtagggtgcactgccgtggtgggcaatctttgcaatgatggcagcattgggatacagtgtgaagagcagca

SEQ ID NO: 29 - DNA, 113 bp, synthetic construct
  source 1..113: mol_type = other DNA
ccctggtgtgccctctgatgttttttaggggattctagggcagtagggtgcactgccgtggtgggcaatcaaacgtatgatggcagcattgggatacagtgtgaagagcagca

SEQ ID NO: 30 - DNA, 113 bp, synthetic construct
  source 1..113: mol_type = other DNA
ccctggtgtgccctctgatgtttttatccccattctaccgctgtgctgggcaatcccgtggtcagcaatctttgcaatgatggcagcattgggatacagtgtgaagagcagca

SEQ ID NO: 31 - DNA, 113 bp, synthetic construct
  source 1..113: mol_type = other DNA
ccctggtgtgccctctgatgttttttaggggattctaccgctgtgctgggcaatcccgtggtcagcaatctttgcatactacgcagcattgggatacagtgtgaagagcagca

SEQ ID NO: 32 - DNA, 113 bp, synthetic construct
  source 1..113: mol_type = other DNA
ccctggtgtgccctctgatgttttttaggggattctaccgctgtgctgggcaatcccgtggtcagcaatcaaacgtatgatggcagcattgggatacagtgtgaagagcagca

SEQ ID NO: 33 - DNA, 113 bp, synthetic construct
  source 1..113: mol_type = other DNA
ccctggtgtgccctctgatgtttttatccccattctacaacagtacggtgaagtgccgtggtcagcaatctttgcaatgatggcagcattgggatacagtgtgaagagcagca

SEQ ID NO: 34 - DNA, 113 bp, synthetic construct
  source 1..113: mol_type = other DNA
ccctggtgtgccctctgatgttttttaggggattctacaacagtacggtgaagtgccgtggtcagcaatcaaacgtatgatggcagcattgggatacagtgtgaagagcagca

SEQ ID NO: 35 - DNA, 113 bp, synthetic construct
  source 1..113: mol_type = other DNA
ccctggtgtgccctctgatgtttttctagatattctacggcagttcatagcaatcccgtagtcaacaatctttgccattatttgagcattgggatacagtgtgaagagcagca

SEQ ID NO: 36 - DNA, 113 bp, synthetic construct
  source 1..113: mol_type = other DNA
ccctggtgtgccctctgatgtttttcggaggattctacggcagttcatagcaatcccgtagtcaacaagggtccccatgatggcagcattgggatacagtgtgaagagcagca

SEQ ID NO: 37 - DNA, 113 bp, synthetic construct
  source 1..113: mol_type = other DNA
ccctggtgtgccctctgatgtttttatccccatcaccctgcagttcatagcaatcccgtagtcaacaacaatatccatgatggcagcattgggatacagtgtgaagagcagca

SEQ ID NO: 38 - DNA, 113 bp, synthetic construct
  source 1..113: mol_type = other DNA
ccctggtgtgccctctgatgtttttctggtgattctacaacagtactgagctatcccgaattcaacaatctttgcaataagcgaagcattgggatacagtgtgaagagcagca

SEQ ID NO: 39 - DNA, 113 bp, synthetic construct
  source 1..113: mol_type = other DNA
ccctggtgtgccctctgatgtttttatccccattcgaaaaaagtactgagctatcccgaattcaacaatctttgcaatcaatcaagcattgggatacagtgtgaagagcagca

SEQ ID NO: 40 - DNA, 113 bp, synthetic construct
  source 1..113: mol_type = other DNA
ccctggtgtgccctctgatgtttttatccccattcgaataaagtactgagctatcccgaattcaacaatcttctattagatggcagcattgggatacagtgtgaagagcagca

SEQ ID NO: 41 - DNA, 113 bp, synthetic construct
  source 1..113: mol_type = other DNA
ccctggtgtgccctctgatgtttttatccccattctacagcagtagtgtgcagtgccgtggtcatcaatctttgcaatgatggcagcattgggatacagtgtgaagagcagca

SEQ ID NO: 42 - DNA, 113 bp, synthetic construct
  source 1..113: mol_type = other DNA
ccctggtgtgccctctgatgttttttaggggattctacagcagtagtgtgcagtgccgtggtcatcaatcaaacgtatgatggcagcattgggatacagtgtgaagagcagca

SEQ ID NO: 43 - DNA, 113 bp, synthetic construct
  source 1..113: mol_type = other DNA
ccctggtgtgccctctgatgtttttcagtagcttctacagcagttcggaggaatcccgaggtcagcaatctttgcaataagagaagcattgggatacagtgtgaagagcagca

SEQ ID NO: 44 - DNA, 113 bp, synthetic construct
  source 1..113: mol_type = other DNA
ccctggtgtgccctctgatgttttttaggggcttctacagcagttcggaggaatcccgaggtcagcaatctttgcatactacgcagcattgggatacagtgtgaagagcagca

SEQ ID NO: 45 - DNA, 113 bp, synthetic construct
  source 1..113: mol_type = other DNA
ccctggtgtgccctctgatgtttttatggaacgtctacagcagttcggaggaatcccgaggtcagcaatctttggtagcttggcagcattgggatacagtgtgaagagcagca

SEQ ID NO: 46 - DNA, 113 bp, synthetic construct
  source 1..113: mol_type = other DNA
ccctggtgtgccctctgatgtttttatccagcgggtacagcagttcggaggaatcccgaggtcagcaatctttggtcttctggcagcattgggatacagtgtgaagagcagca

SEQ ID NO: 47 - DNA, 113 bp, synthetic construct
  source 1..113: mol_type = other DNA
ccctggtgtgccctctgatgtttcaacggcccttctacagcagttcggaggaatcccgaggtcagcaatcttaccctggatggcagcattgggatacagtgtgaagagcagca

SEQ ID NO: 48 - DNA, 113 bp, synthetic construct
  source 1..113: mol_type = other DNA
ccctggtgtgccctctgatgtttttattgagcctctacagcagttcggaggaatcccgaggtcagcaatcttaaattagatggcagcattgggatacagtgtgaagagcagca

SEQ ID NO: 49 - DNA, 113 bp, synthetic construct
  source 1..113: mol_type = other DNA
ccctggtgtgccctctgatgttttttaggggcttctacagcagttcggaggaatcccgaggtcagcaatcaaacgtatgatggcagcattgggatacagtgtgaagagcagca

SEQ ID NO: 50 - DNA, 113 bp, synthetic construct
  source 1..113: mol_type = other DNA
ccctggtgtgccctctgatgtttttatgaggcgtctacagcagttcggaggaatcccgaggtcagcaagactaacaatgatggcagcattgggatacagtgtgaagagcagca

SEQ ID NO: 51 - DNA, 113 bp, synthetic construct
  source 1..113: mol_type = other DNA
ccctggtgtgccctctgatgtttttattgagcctctacagcagttcggaggaatcccgaggtcagcttagcctgcaatgatggcagcattgggatacagtgtgaagagcagca

SEQ ID NO: 52 - DNA, 45 bp, synthetic construct
  source 1..45: mol_type = other DNA
attctacggcagtactgggacatcccgtggtcgtgaatctttgca

SEQ ID NO: 53 - DNA, 45 bp, synthetic construct
  source 1..45: mol_type = other DNA
attctacagcaggactgttcagtcccgtagtcggaaatctttgca

SEQ ID NO: 54 - DNA, 45 bp, synthetic construct
  source 1..45: mol_type = other DNA
attctacagcagggctcagacatcccgtggtcgccaatctttgca

SEQ ID NO: 55 - DNA, 45 bp, synthetic construct
  source 1..45: mol_type = other DNA
attctacagcaagactgggacatcccgtggtcggcattctttgca

SEQ ID NO: 56 - DNA, 45 bp, synthetic construct
  source 1..45: mol_type = other DNA
attctacagcagtactcaggaaatccgtggtcggatatctttgca

SEQ ID NO: 57 - DNA, 45 bp, synthetic construct
  source 1..45: mol_type = other DNA
attctacagcagtactgttcagttccgaggtcataaatctttgca

SEQ ID NO: 58 - DNA, 45 bp, synthetic construct
  source 1..45: mol_type = other DNA
attctacagcaggactgggctctcccgtggtcatcaatcgttgca

SEQ ID NO: 59 - DNA, 45 bp, synthetic construct
  source 1..45: mol_type = other DNA
attctacagcggtactgggacatcccgtggtcggaaaactttgca

SEQ ID NO: 60 - DNA, 45 bp, synthetic construct
  source 1..45: mol_type = other DNA
attctatcgcagtattggggaatcccgtggtcttcaatctttgca

SEQ ID NO: 61 - DNA, 45 bp, synthetic construct
  source 1..45: mol_type = other DNA
attctacagcagtactgggctttcccgtgttaaccaatctttgca

SEQ ID NO: 62 - DNA, 45 bp, synthetic construct
  source 1..45: mol_type = other DNA
attctacagcagtactgtggaaatccgtggttgtcaatctttgca

SEQ ID NO: 63 - DNA, 45 bp, synthetic construct
  source 1..45: mol_type = other DNA
attctacagcaggactcagcacagccgaagttgccaatctttgca

SEQ ID NO: 64 - DNA, 45 bp, synthetic construct
  source 1..45: mol_type = other DNA
gttctagagcaggactgtccagtcccgtagtcggcaatctttgca

SEQ ID NO: 65 - DNA, 45 bp, synthetic construct
  source 1..45: mol_type = other DNA
attctacggcagtactggggaaatccgtggtcggcaatctttgca

SEQ ID NO: 66 - DNA, 45 bp, synthetic construct
  source 1..45: mol_type = other DNA
attctaccgcagtactgtgacatcccgtggtcttcaatctttgca

SEQ ID NO: 67 - DNA, 45 bp, synthetic construct
  source 1..45: mol_type = other DNA
attctacagcaggagtgtgcagtcccgaagacggcaatctttgca

SEQ ID NO: 68 - DNA, 45 bp, synthetic construct
  source 1..45: mol_type = other DNA
attctacggcaggactggggaaatccgtggtcggccatctttgca

SEQ ID NO: 69 - DNA, 45 bp, synthetic construct
  source 1..45: mol_type = other DNA
attctacagcggtactcaccagtcccgaagtcatcaatctttgcc

SEQ ID NO: 70 - DNA, 45 bp, synthetic construct
  source 1..45: mol_type = other DNA
attctaccgcagtactgcgaaatcccgaggtcggcaatctttgca

SEQ ID NO: 71 - DNA, 45 bp, synthetic construct
  source 1..45: mol_type = other DNA
attctactgcaggacttaggagtcccgtggtcggcattctttgca

SEQ ID NO: 72 - DNA, 45 bp, synthetic construct
  source 1..45: mol_type = other DNA
attctagggcagtactgggacatcccgtggtcggcaatctttgca

SEQ ID NO: 73 - DNA, 45 bp, synthetic construct
  source 1..45: mol_type = other DNA
attctacagcagtactcaccagtcccgtggtgggcaatctttgca

SEQ ID NO: 74 - DNA, 45 bp, synthetic construct
  source 1..45: mol_type = other DNA
attctacagcagtgctcaccagtcccgtggtcggcaatctttgca

SEQ ID NO: 75 - DNA, 45 bp, synthetic construct
  source 1..45: mol_type = other DNA
attctagagcagtactggggagttccgtggtcgtcaatctttgca

SEQ ID NO: 76 - DNA, 45 bp, synthetic construct
  source 1..45: mol_type = other DNA
attctacagcaggagtgggcagtcccgtggtcggcaatctttgca

SEQ ID NO: 77 - DNA, 45 bp, synthetic construct
  source 1..45: mol_type = other DNA
attctacggcaggactggggaaatccgtggtcgggaatctttgca

SEQ ID NO: 78 - DNA, 45 bp, synthetic construct
  source 1..45: mol_type = other DNA
attctacagcaggagtgtgcagtcccgtggtcggcaatctttgca

SEQ ID NO: 79 - DNA, 45 bp, synthetic construct
  source 1..45: mol_type = other DNA
attctacatctgtattgggacatcccgtggtcggcaatctttgca

SEQ ID NO: 80 - DNA, 45 bp, synthetic construct
  source 1..45: mol_type = other DNA
attctacagcaagactgggcagtcccgtagacggcaatctttgca

SEQ ID NO: 81 - DNA, 45 bp, synthetic construct
  source 1..45: mol_type = other DNA
attctagagcagtactgtgacatcccgtggtcggcaatctttgca

SEQ ID NO: 82 - DNA, 45 bp, synthetic construct
  source 1..45: mol_type = other DNA
attctatagcaggactgagcactcccgtggtcgacaatctttgca

SEQ ID NO: 83 - DNA, 45 bp, synthetic construct
  source 1..45: mol_type = other DNA
attctaccgcagtacttatcagtcccgtggtcggcaatctttgca

SEQ ID NO: 84 - DNA, 45 bp, synthetic construct
  source 1..45: mol_type = other DNA
gttctacagcaggcttgggcagtcccgtggtcggcaatctttgca

SEQ ID NO: 85 - DNA, 45 bp, synthetic construct
  source 1..45: mol_type = other DNA
attctacagcagtactcagctttcccgtggtcgacaatctttgca

SEQ ID NO: 86 - DNA, 45 bp, synthetic construct
  source 1..45: mol_type = other DNA
attctacggtaggacggagcagtcccgtggtcggcaatctttgca

SEQ ID NO: 87 - DNA, 45 bp, synthetic construct
  source 1..45: mol_type = other DNA
attctacagcagtactcaccagtcccgtggtcggcaatctttgca

SEQ ID NO: 88 - DNA, 45 bp, synthetic construct
  source 1..45: mol_type = other DNA
attctacagccgtacagagcagtcccgtggacggcaatctttgca

SEQ ID NO: 89 - DNA, 45 bp, synthetic construct
  source 1..45: mol_type = other DNA
attctacggcaggactgggcagtcccgtagtcggcaatctttgca

SEQ ID NO: 90 - DNA, 45 bp, synthetic construct
  source 1..45: mol_type = other DNA
attctagtgcaggactgggacatcccgtagtcggcaatctttgca

SEQ ID NO: 91 - DNA, 45 bp, synthetic construct
  source 1..45: mol_type = other DNA
attctacagtggtactcaccagtcccgtggtcggcaatctttgca

SEQ ID NO: 92 - DNA, 45 bp, synthetic construct
  source 1..45: mol_type = other DNA
attctacagcagtactcagaagttccgtggtcgccaatctttgca

SEQ ID NO: 93 - DNA, 45 bp, synthetic construct
  source 1..45: mol_type = other DNA
attctacagcggtactgtttaatcccgtggtcggcaatcgttgca

SEQ ID NO: 94 - DNA, 45 bp, synthetic construct
  source 1..45: mol_type = other DNA
attctatggcagtactgtgcagtcccgtggtcgacaatctttgca

SEQ ID NO: 95 - DNA, 45 bp, synthetic construct
  source 1..45: mol_type = other DNA
attctacaggaggactgagcagtcccgagttgggcaatctttgca

SEQ ID NO: 96 - DNA, 45 bp, synthetic construct
  source 1..45: mol_type = other DNA
attctagagcagtactgcgcaaatccgtggtcggcaatctttgca

SEQ ID NO: 97 - DNA, 45 bp, synthetic construct
  source 1..45: mol_type = other DNA
attctacagcaggactgaggagtcccgaggccggcaatctttgca

SEQ ID NO: 98 - DNA, 45 bp, synthetic construct
  source 1..45: mol_type = other DNA
attctacaggagtactgattaaatccgtggttgggaatctttgca

SEQ ID NO: 99 - DNA, 45 bp, synthetic construct
  source 1..45: mol_type = other DNA
attctacaggaggactgggacatcccgtggtcgctaatctttgca

SEQ ID NO: 100 - DNA, 45 bp, synthetic construct
  source 1..45: mol_type = other DNA
attctacagccgtacagggcagtcccgtggacggcaatctttgca

SEQ ID NO: 101 - DNA, 45 bp, synthetic construct
  source 1..45: mol_type = other DNA
attctacagcagtacggtccagtcccgtggtcggcaatctttgca

SEQ ID NO: 102 - DNA, 45 bp, synthetic construct
  source 1..45: mol_type = other DNA
attctacagcagtactgagcaatgccgtagtcagcaatctttgca

SEQ ID NO: 103 - DNA, 45 bp, synthetic construct
  source 1..45: mol_type = other DNA
attctacggcggtactgaccaatcccgtagttagcaatctttgca

SEQ ID NO: 104 - DNA, 45 bp, synthetic construct
  source 1..45: mol_type = other DNA
attctacagtaggactgagcactgccgagctgggcaatctttgca

SEQ ID NO: 105 - DNA, 45 bp, synthetic construct
  source 1..45: mol_type = other DNA
cttctacagcagttcggaggaatcccgaggtcagcaatctttgca

SEQ ID NO: 106 - RNA, 113 nt, synthetic construct
  source 1..113: mol_type = other RNA
ccctggtgtgccctctgatgttttttaggggattctacagcaggactgagcaatcttgtggtcagcaatatttgcatactacgcagcattgggatacagtgtgaaaagcagca

SEQ ID NO: 107 - RNA, 113 nt, synthetic construct
  source 1..113: mol_type = other RNA
ccctggtgtgccctctgatgttttttaggggattctacagcaggactgagcaatggagtggtcagcaatatttgcatactacgcagcattgggatacagtgtgaaaagcagca

SEQ ID NO: 108 - RNA, 113 nt, synthetic construct
  source 1..113: mol_type = other RNA
ccctggtgtgccctctgatgttttttaggggattctacagcacgactgagcagtgcgttagtcagccatctttgcatactacgcagcattgggatacagtgtgaaaagcagca

SEQ ID NO: 109 - RNA, 113 nt, synthetic construct
  source 1..113: mol_type = other RNA
ccctggtgtgccctctgatgttttttaggggattctacagcattactcagcagtgcgttagtcagcactctttgcatactacgcagcattgggatacagtgtgaaaagcagca

SEQ ID NO: 110 - RNA, 113 nt, synthetic construct
  source 1..113: mol_type = other RNA
ccctggtgtgccctctgatgttttttaggggattctacagcacgactgagcagtgcgttagtcggcaatctttgcatactacgcagcattgggatacagtgtgaaaagcagca

SEQ ID NO: 111 - RNA, 113 nt, synthetic construct
  source 1..113: mol_type = other RNA
ccctggtgtgccctctgatgttttttaggggattctacagcaatactcagcagtgcgtgagtcagcaatctttgcatactacgcagcattgggatacagtgtgaaaagcagca

SEQ ID NO: 112 - RNA, 113 nt, synthetic construct
  source 1..113: mol_type = other RNA
ccctggtgtgccctctgatgttttttaggggattctacagcacgactgagcagtgcgctagtcggcaatctttgcatactacgcagcattgggatacagtgtgaaaagcagca

SEQ ID NO: 113 - RNA, 113 nt, synthetic construct
  source 1..113: mol_type = other RNA
ccctggtgtgccctctgatgttttttaggggattctacagcacgactgagcagtgcgctagtcagcaatatttgcatactacgcagcattgggatacagtgtgaaaagcagca

SEQ ID NO: 114 - RNA, 113 nt, synthetic construct
  source 1..113: mol_type = other RNA
ccctggtgtgccctctgatgttttttaggggattctacagcaatactcagcagtgcgctagtcagccatctttgcatactacgcagcattgggatacagtgtgaaaagcagca

SEQ ID NO: 115 - RNA, 113 nt, synthetic construct
  source 1..113: mol_type = other RNA
ccctggtgtgccctctgatgttttttaggggattctacagcatgactgagcagtcccgtagtcggcaatctttgcatactacgcagcattgggatacagtgtgaaaagcagca

SEQ ID NO: 116 - RNA, 113 nt, synthetic construct
  source 1..113: mol_type = other RNA
ccctggtgtgccctctgatgttttttaggggattctacagcaatactcagcagttccgtagtcagcaatatttgcatactacgcagcattgggatacagtgtgaaaagcagca

SEQ ID NO: 117 - RNA, 113 nt, synthetic construct
  source 1..113: mol_type = other RNA
ccctggtgtgccctctgatgttttttaggggattctacagcattactcagcagtcccgtagtcagcaatatttgcatactacgcagcattgggatacagtgtgaaaagcagca

SEQ ID NO: 118 - RNA, 113 nt, synthetic construct
  source 1..113: mol_type = other RNA
ccctggtgtgccctctgatgttttttaggggattctacagcattactcagcaaatccgtagtcagaaatctttgcatactacgcagcattgggatacagtgtgaaaagcagca

SEQ ID NO: 119 - RNA, 113 nt, synthetic construct
  source 1..113: mol_type = other RNA
ccctggtgtgccctctgatgttttttaggggattctagagaggtactgtgccatcccgtggtcggccatctttgcatactacgcagcattgggatacagtgtgaaaagcagca

SEQ ID NO: 120 - RNA, 113 nt, synthetic construct
  source 1..113: mol_type = other RNA
ccctggtgtgccctctgatgttttttaggggattctagagcaggacggtgcagtcccgtggtcggcaatctttgcatactacgcagcattgggatacagtgtgaaaagcagca

SEQ ID NO: 121 - RNA, 113 nt, synthetic construct
  source 1..113: mol_type = other RNA
ccctggtgtgccctctgatgttttttaggggattctacaggaggactgggcagtcccgtggtcgcccttctttgcatactacgcagcattgggatacagtgtgaaaagcagca

SEQ ID NO: 122 - RNA, 113 nt, synthetic construct
  source 1..113: mol_type = other RNA
ccctggtgtgccctctgatgttttttaggggattctacagcggtactgtgcaaatccgtggtcggtcatctttgcatactacgcagcattgggatacagtgtgaaaagcagca

SEQ ID NO: 123 - RNA, 113 nt, synthetic construct
  source 1..113: mol_type = other RNA
ccctggtgtgccctctgatgttttttaggggattctacggcaggactggggaaatccgtggtcggccatctttgcatactacgcagcattgggatacagtgtgaaaagcagca

SEQ ID NO: 124 - RNA, 113 nt, synthetic construct
  source 1..113: mol_type = other RNA
ccctggtgtgccctctgatgttttttaggggattctacagcggtactgcgcagtcccgtggtggtccatctttgcatactacgcagcattgggatacagtgtgaaaagcagca

SEQ ID NO: 125 - RNA, 113 nt, synthetic construct
  source 1..113: mol_type = other RNA
ccctggtgtgccctctgatgttttttaggggattctatagcaggaatgaggaaatccgtggtcggcaatctttgcatactacgcagcattgggatacagtgtgaaaagcagca

SEQ ID NO: 126 - RNA, 113 nt, synthetic construct
  source 1..113: mol_type = other RNA
ccctggtgtgccctctgatgttttttaggggattctacagcagtactcaggaaatccgtggtcggatatctttgcatactacgcagcattgggatacagtgtgaaaagcagca

SEQ ID NO: 127 - RNA, 113 nt, synthetic construct
  source 1..113: mol_type = other RNA
ccctggtgtgccctctgatgttttttaggggattctaaagcaggagtgagcagatccgtggtcggcaatctttgcatactacgcagcattgggatacagtgtgaaaagcagca

SEQ ID NO: 128 - RNA, 113 nt, synthetic construct
  source 1..113: mol_type = other RNA
ccctggtgtgccctctgatgttttttaggggattctacaggtgtactgtgaaatcccgtggtcataaatctttgcatactacgcagcattgggatacagtgtgaaaagcagca

SEQ ID NO: 129 - RNA, 113 nt, synthetic construct
  source 1..113: mol_type = other RNA
ccctggtgtgccctctgatgttttttaggggattctacagcagtactgtccagtcccgtggtcgtaaatctttgcatactacgcagcattgggatacagtgtgaaaagcagca

SEQ ID NO: 130 - RNA, 113 nt, synthetic construct
  source 1..113: mol_type = other RNA
ccctggtgtgccctctgatgttttttaggggattctacatcaggactgagcttttccgaggtcggctatctttgcatactacgcagcattgggatacagtgtgaaaagcagca

SEQ ID NO: 131 - RNA, 113 nt, synthetic construct
  source 1..113: mol_type = other RNA
ccctggtgtgccctctgatgttttttaggggattctacatcagtactgggcagttccgtagtcggcaatctttgcatactacgcagcattgggatacagtgtgaaaagcagca

SEQ ID NO: 132 - RNA, 113 nt, synthetic construct
  source 1..113: mol_type = other RNA
ccctggtgtgccctctgatgttttttaggggattctacagcagtactgggcagtgcgctagtcggcactctttgcatactacgcagcattgggatacagtgtgaaaagcagca

SEQ ID NO: 133 - RNA, 113 nt, synthetic construct
  source 1..113: mol_type = other RNA
ccctggtgtgccctctgatgttttttaggggattctacagcaatactcaccagtcccgatgtaagcaatctttgcatactacgcagcattgggatacagtgtgaaaagcagca

SEQ ID NO: 134 - RNA, 113 nt, synthetic construct
  source 1..113: mol_type = other RNA
ccctggtgtgccctctgatgttttttaggggattctacggcagtactgtgaaatcccgatgtcggcaatctttgcatactacgcagcattgggatacagtgtgaaaagcagca

SEQ ID NO: 135 - RNA, 113 nt, synthetic construct
  source 1..113: mol_type = other RNA
ccctggtgtgccctctgatgttttttaggggattctaccgcaggacggagctatcccgagttaggcaatctttgcatactacgcagcattgggatacagtgtgaaaagcagca

SEQ ID NO: 136 - RNA, 113 nt, synthetic construct
  source 1..113: mol_type = other RNA
ccctggtgtgccctctgatgttttttaggggattctacggcatgactgatcagtcccgtggtcggaccccgatgcatactacgcagcattgggatacagtgtgaaaagcagca

SEQ ID NO: 137 - RNA, 113 nt, synthetic construct
  source 1..113: mol_type = other RNA
ccctggtgtgccctctgatgttttttaggggattctacggcaagactgggcagtcccgtggtcggacccccgtgcatactacgcagcattgggatacagtgtgaaaagcagca

SEQ ID NO: 138 - RNA, 113 nt, synthetic construct
  source 1..113: mol_type = other RNA
ccctggtgtgccctctgatgttttttaggggattctacggcaggactgggctttcccgtggtcggacctccgtgcatactacgcagcattgggatacagtgtgaaaagcagca

SEQ ID NO: 139 - RNA, 113 nt, synthetic construct
  source 1..113: mol_type = other RNA
ccctggtgtgccctctgatgttttttaggggattctacggtaggactgatcagtcccgtggtcgaaccacgttgcatactacgcagcattgggatacagtgtgaaaagcagca

SEQ ID NO: 140 - RNA, 113 nt, synthetic construct
  source 1..113: mol_type = other RNA
ccctggtgtgccctctgatgttttttaggggattctacggcaagactgagcagtcccgtggtggaaccccgttgcatactacgcagcattgggatacagtgtgaaaagcagca

SEQ ID NO: 141 - RNA, 113 nt, synthetic construct
  source 1..113: mol_type = other RNA
ccctggtgtgccctctgatgttttttaggggatcctacggcagtactgatcaaatccgtggtggcaccccgttgcatactacgcagcattgggatacagtgtgaaaagcagca

SEQ ID NO: 142 - RNA, 113 nt, synthetic construct
  source 1..113: mol_type = other RNA
ccctggtgtgccctctgatgttttttaggggattctacagcaggactgaccagttccgtggtggcacctccgtgcatactacgcagcattgggatacagtgtgaaaagcagca

SEQ ID NO: 143 - RNA, 113 nt, synthetic construct
  source 1..113: mol_type = other RNA
ccctggtgtgccctctgatgttttttaggggattctacggcagtactgagaaatcccgtggtggcaccccgttgcatactacgcagcattgggatacagtgtgaaaagcagca

SEQ ID NO: 144 - RNA, 113 nt, synthetic construct
  source 1..113: mol_type = other RNA
ccctggtgtgccctctgatgttttttaggggattctacagcagtcctgaccagtcccgtggtcgtacctccgtgcatactacgcagcattgggatacagtgtgaaaagcagca

SEQ ID NO: 145 - RNA, 113 nt, synthetic construct
  source 1..113: mol_type = other RNA
ccctggtgtgccctctgatgttttttaggggcttctacagcaggactgatcaaatccgtggtggaaccccgttgcatactacgcagcattgggatacagtgtgaaaagcagca

SEQ ID NO: 146 - RNA, 113 nt, synthetic construct
  source 1..113: mol_type = other RNA
ccctggtgtgccctctgatgttttttaggggattctacggcagtactgagacatcccgtggtggcacccccgtgcatactacgcagcattgggatacagtgtgaaaagcagca

SEQ ID NO: 147 - RNA, 113 nt, synthetic construct
  source 1..113: mol_type = other RNA
ccctggtgtgccctctgatgttttttaggggattctaccgcagtactcaccagtcgccgtgtcgtcaatctttgcatactacgcagcattgggatacagtgtgaaaagcagca

SEQ ID NO: 148 - RNA, 113 nt, synthetic construct
  source 1..113: mol_type = other RNA
ccctggtgtgccctctgatgttttttaggggattctaccgcagtactcaccagtcgccgtgtcggcaatctttgcatactacgcagcattgggatacagtgtgaaaagcagca

SEQ ID NO: 149 - DNA, 113 bp, synthetic construct
  source 1..113: mol_type = other DNA
ccctggtgtgccctctgatgttttttaggggattctacagcagtactgagcaatgccgtagtcagcaatctttgcatactacgcagcattgggatacagtgtgaaaagcagca

SEQ ID NO: 150 - DNA, 113 bp, synthetic construct
  source 1..113: mol_type = other DNA
ccctggtgtgccctctgatgttttttaggggattctacaacagtactgagctatcccgaattcaacaatctttgcatactacgcagcattgggatacagtgtgaaaagcagca

SEQ ID NO: 151 - DNA, 113 bp, synthetic construct
  source 1..113: mol_type = other DNA
ccctggtgtgccctctgatgttttttaggggattctacatctgtagtgagcaattccgtgctcagcaatctttgcatactacgcagcattgggatacagtgtgaaaagcagca

SEQ ID NO: 152 - DNA, 113 bp, synthetic construct; identical to SEQ ID NO: 149
  source 1..113: mol_type = other DNA

SEQ ID NO: 153 - DNA, 113 bp, synthetic construct
  source 1..113: mol_type = other DNA
ccctggtgtgccctctgatgttttttaggggcttctacagcagttcggaggaatcccgaggtcagcaatctttgcatactacgcagcattgggatacagtgtgaaaagcagca

SEQ ID NO: 154 - DNA, 113 bp, synthetic construct; identical to SEQ ID NO: 150
  source 1..113: mol_type = other DNA

SEQ ID NO: 155 - DNA, 113 bp, synthetic construct
  source 1..113: mol_type = other DNA
ccctggtgtgccctctgatgttttttaggggattctaccgctgtgctgggcaatcccgtggtcagcaatctttgcatactacgcagcattgggatacagtgtgaaaagcagca

SEQ ID NO: 156 - DNA, 113 bp, synthetic construct
  source 1..113: mol_type = other DNA
ccctggtgtgccctctgatgttttttaggggattctacagtaggactgagcactgccgagctgggcaatctttgcatactacgcagcattgggatacagtgtgaaaagcagca

SEQ ID NO: 157 - DNA, 113 bp, synthetic construct; identical to SEQ ID NO: 151
  source 1..113: mol_type = other DNA

SEQ ID NO: 158 - DNA, 113 bp, synthetic construct
  source 1..113: mol_type = other DNA
ccctggtgtgccctctgatgttttttaggggattctacggtaggactgatcagtcccgtggtcgaaccacgttgcatactacgcagcattgggatacagtgtgaaaagcagca

SEQ ID NO: 159 - DNA, 113 bp, synthetic construct
  source 1..113: mol_type = other DNA
ccctggtgtgccctctgatgttttttaggggattctacggcaagactgagcagtcccgtggtggaaccccgttgcatactacgcagcattgggatacagtgtgaaaagcagca

SEQ ID NO: 160 - DNA, 113 bp, synthetic construct
  source 1..113: mol_type = other DNA
ccctggtgtgccctctgatgttttttaggggatcctacggcagtactgatcaaatccgtggtggcaccccgttgcatactacgcagcattgggatacagtgtgaaaagcagca

SEQ ID NO: 161 - DNA, 113 bp, synthetic construct
  source 1..113: mol_type = other DNA
ccctggtgtgccctctgatgttttttaggggattctacagcaggactgaccagttccgtggtggcacctccgtgcatactacgcagcattgggatacagtgtgaaaagcagca

SEQ ID NO: 162 - DNA, 113 bp, synthetic construct
  source 1..113: mol_type = other DNA
ccctggtgtgccctctgatgttttttaggggattctacggcagtactgagaaatcccgtggtggcaccccgttgcatactacgcagcattgggatacagtgtgaaaagcagca

SEQ ID NO: 163 - DNA, 113 bp, synthetic construct
  source 1..113: mol_type = other DNA
ccctggtgtgccctctgatgttttttaggggattctacagcagtcctgaccagtcccgtggtcgtacctccgtgcatactacgcagcattgggatacagtgtgaaaagcagca

SEQ ID NO: 164 - DNA, 113 bp, synthetic construct
  source 1..113: mol_type = other DNA
ccctggtgtgccctctgatgttttttaggggcttctacagcaggactgatcaaatccgtggtggaaccccgttgcatactacgcagcattgggatacagtgtgaaaagcagca

SEQ ID NO: 165 - DNA, 113 bp, synthetic construct
  source 1..113: mol_type = other DNA
ccctggtgtgccctctgatgttttttaggggattctacggcagtactgagacatcccgtggtggcacccccgtgcatactacgcagcattgggatacagtgtgaaaagcagca

SEQ ID NO: 166 - DNA, 113 bp, synthetic construct
  source 1..113: mol_type = other DNA
ccctggtgtgccctctgatgttttttaggggattctaccgcagtactcaccagtcgccgtgtcgtcaatctttgcatactacgcagcattgggatacagtgtgaaaagcagca

SEQ ID NO: 167 - DNA, 113 bp, synthetic construct
  source 1..113: mol_type = other DNA
ccctggtgtgccctctgatgttttttaggggattctaccgcagtactcaccagtcgccgtgtcggcaatctttgcatactacgcagcattgggatacagtgtgaaaagcagca

SEQ ID NO: 168 - DNA, 113 bp, synthetic construct
  source 1..113: mol_type = other DNA
ccctggtgtgccctctgatgttttttaggggattctacagcattactcagcagtgcgttagtcagcactctttgcatactacgcagcattgggatacagtgtgaaaagcagca

SEQ ID NO: 169 - DNA, 113 bp, synthetic construct
  source 1..113: mol_type = other DNA
ccctggtgtgccctctgatgttttttaggggattctacagcacgactgagcagtgcgttagtcggcaatctttgcatactacgcagcattgggatacagtgtgaaaagcagca

SEQ ID NO: 170 - DNA, 113 bp, synthetic construct
  source 1..113: mol_type = other DNA
ccctggtgtgccctctgatgttttttaggggattctacagcaatactcagcagtgcgtgagtcagcaatctttgcatactacgcagcattgggatacagtgtgaaaagcagca

SEQ ID NO: 171 - DNA, 113 bp, synthetic construct
  source 1..113: mol_type = other DNA
ccctggtgtgccctctgatgttttttaggggattctacagcacgactgagcagtgcgctagtcggcaatctttgcatactacgcagcattgggatacagtgtgaaaagcagca

SEQ ID NO: 172 - DNA, 113 bp, synthetic construct
  source 1..113: mol_type = other DNA
ccctggtgtgccctctgatgttttttaggggattctacagcagtactgtccagtcccgtggtcgtaaatctttgcatactacgcagcattgggatacagtgtgaaaagcagca

SEQ ID NO: 173 - DNA, 113 bp, synthetic construct
  source 1..113: mol_type = other DNA
ccctggtgtgccctctgatgttttttaggggattctacatcaggactgagcttttccgaggtcggctatctttgcatactacgcagcattgggatacagtgtgaaaagcagca

SEQ ID NO: 174 - DNA, 113 bp, synthetic construct
  source 1..113: mol_type = other DNA
ccctggtgtgccctctgatgttttttaggggattctacatcagtactgggcagttccgtagtcggcaatctttgcatactacgcagcattgggatacagtgtgaaaagcagca

SEQ ID NO: 175 - DNA, 113 bp, synthetic construct
  source 1..113: mol_type = other DNA
ccctggtgtgccctctgatgttttttaggggattctacagcagtactgggcagtgcgctagtcggcactctttgcatactacgcagcattgggatacagtgtgaaaagcagca

SEQ ID NO: 176 - DNA, 113 bp, synthetic construct
  source 1..113: mol_type = other DNA
ccctggtgtgccctctgatgttttttaggggattctacagcaatactcaccagtcccgatgtaagcaatctttgcatactacgcagcattgggatacagtgtgaaaagcagca

SEQ ID NO: 177 - DNA, 113 bp, synthetic construct
  source 1..113: mol_type = other DNA
ccctggtgtgccctctgatgttttttaggggattctacggcagtactgtgaaatcccgatgtcggcaatctttgcatactacgcagcattgggatacagtgtgaaaagcagca

SEQ ID NO: 178 - DNA, 113 bp, synthetic construct
  source 1..113: mol_type = other DNA
ccctggtgtgccctctgatgttttttaggggattctaccgcaggacggagctatcccgagttaggcaatctttgcatactacgcagcattgggatacagtgtgaaaagcagca

SEQ ID NO: 179 - DNA, 113 bp, synthetic construct
  source 1..113: mol_type = other DNA
ccctggtgtgccctctgatgttttttaggggattctacggcatgactgatcagtcccgtggtcggaccccgatgcatactacgcagcattgggatacagtgtgaaaagcagca

SEQ ID NO: 180 - DNA, 113 bp, synthetic construct
  source 1..113: mol_type = other DNA
ccctggtgtgccctctgatgttttttaggggattctacggcaagactgggcagtcccgtggtcggacccccgtgcatactacgcagcattgggatacagtgtgaaaagcagca

SEQ ID NO: 181 - DNA, 113 bp, synthetic construct
  source 1..113: mol_type = other DNA
ccctggtgtgccctctgatgttttttaggggattctacggcaggactgggctttcccgtggtcggacctccgtgcatactacgcagcattgggatacagtgtgaaaagcagca

SEQ ID NO: 182 - DNA, 113 bp, synthetic construct
  source 1..113: mol_type = other DNA
ccctggtgtgccctctgatgttttttaggggattctacagcacgactgagcagtgcgctagtcagcaatatttgcatactacgcagcattgggatacagtgtgaaaagcagca

SEQ ID NO: 183 - DNA, 113 bp, synthetic construct
  source 1..113: mol_type = other DNA
ccctggtgtgccctctgatgttttttaggggattctacagcaatactcagcagtgcgctagtcagccatctttgcatactacgcagcattgggatacagtgtgaaaagcagca

SEQ ID NO: 184 - DNA, 113 bp, synthetic construct
  source 1..113: mol_type = other DNA
ccctggtgtgccctctgatgttttttaggggattctacagcatgactgagcagtcccgtagtcggcaatctttgcatactacgcagcattgggatacagtgtgaaaagcagca

SEQ ID NO: 185 - DNA, 113 bp, synthetic construct
  source 1..113: mol_type = other DNA
ccctggtgtgccctctgatgttttttaggggattctacagcaatactcagcagttccgtagtcagcaatatttgcatactacgcagcattgggatacagtgtgaaaagcagca

SEQ ID NO: 186 - DNA, 113 bp, synthetic construct
  source 1..113: mol_type = other DNA
ccctggtgtgccctctgatgttttttaggggattctagagaggtactgtgccatcccgtggtcggccatctttgcatactacgcagcattgggatacagtgtgaaaagcagca

SEQ ID NO: 187 - DNA, 113 bp, synthetic construct
  source 1..113: mol_type = other DNA
ccctggtgtgccctctgatgttttttaggggattctagagcaggacggtgcagtcccgtggtcggcaatctttgcatactacgcagcattgggatacagtgtgaaaagcagca

SEQ ID NO: 188 - DNA, 113 bp, synthetic construct
  source 1..113: mol_type = other DNA
ccctggtgtgccctctgatgttttttaggggattctacaggaggactgggcagtcccgtggtcgcccttctttgcatactacgcagcattgggatacagtgtgaaaagcagca

SEQ ID NO: 189 - DNA, 113 bp, synthetic construct
  source 1..113: mol_type = other DNA
ccctggtgtgccctctgatgttttttaggggattctacagcggtactgtgcaaatccgtggtcggtcatctttgcatactacgcagcattgggatacagtgtgaaaagcagca

SEQ ID NO: 190 - DNA, 113 bp, synthetic construct
  source 1..113: mol_type = other DNA
ccctggtgtgccctctgatgttttttaggggattctacggcaggactggggaaatccgtggtcggccatctttgcatactacgcagcattgggatacagtgtgaaaagcagca

SEQ ID NO: 191 - DNA, 113 bp, synthetic construct
  source 1..113: mol_type = other DNA
ccctggtgtgccctctgatgttttttaggggattctacagcggtactgcgcagtcccgtggtggtccatctttgcatactacgcagcattgggatacagtgtgaaaagcagca

SEQ ID NO: 192 - DNA, 113 bp, synthetic construct
  source 1..113: mol_type = other DNA
ccctggtgtgccctctgatgttttttaggggattctatagcaggaatgaggaaatccgtggtcggcaatctttgcatactacgcagcattgggatacagtgtgaaaagcagca

SEQ ID NO: 193 - DNA, 113 bp, synthetic construct
  source 1..113: mol_type = other DNA
ccctggtgtgccctctgatgttttttaggggattctacagcagtactcaggaaatccgtggtcggatatctttgcatactacgcagcattgggatacagtgtgaaaagcagca

SEQ ID NO: 194 - DNA, 113 bp, synthetic construct
  source 1..113: mol_type = other DNA
ccctggtgtgccctctgatgttttttaggggattctaaagcaggagtgagcagatccgtggtcggcaatctttgcatactacgcagcattgggatacagtgtgaaaagcagca

SEQ ID NO: 195 - DNA, 113 bp, synthetic construct
  source 1..113: mol_type = other DNA
ccctggtgtgccctctgatgttttttaggggattctacaggtgtactgtgaaatcccgtggtcataaatctttgcatactacgcagcattgggatacagtgtgaaaagcagca

SEQ ID NO: 196 - DNA, 113 bp, synthetic construct
  source 1..113: mol_type = other DNA
ccctggtgtgccctctgatgttttttaggggattctacagcattactcagcagtcccgtagtcagcaatatttgcatactacgcagcattgggatacagtgtgaaaagcagca

SEQ ID NO: 197 - DNA, 113 bp, synthetic construct
  source 1..113: mol_type = other DNA
ccctggtgtgccctctgatgttttttaggggattctacagcattactcagcaaatccgtagtcagaaatctttgcatactacgcagcattgggatacagtgtgaaaagcagca

SEQ ID NO: 198 - DNA, 113 bp, synthetic construct
  source 1..113: mol_type = other DNA
ccctggtgtgccctctgatgttttttaggggattctacagcaggactgagcaatcttgtggtcagcaatatttgcatactacgcagcattgggatacagtgtgaaaagcagca

SEQ ID NO: 199 - DNA, 113 bp, synthetic construct
  source 1..113: mol_type = other DNA
ccctggtgtgccctctgatgttttttaggggattctacagcaggactgagcaatggagtggtcagcaatatttgcatactacgcagcattgggatacagtgtgaaaagcagca

SEQ ID NO: 200 - DNA, 113 bp, synthetic construct
  source 1..113: mol_type = other DNA
ccctggtgtgccctctgatgttttttaggggattctacagcacgactgagcagtgcgttagtcagccatctttgcatactacgcagcattgggatacagtgtgaaaagcagca

SEQ ID NO: 201 - DNA, 113 bp, synthetic construct; identical to SEQ ID NO: 188
  source 1..113: mol_type = other DNA

SEQ ID NO: 202 - DNA, 113 bp, synthetic construct; identical to SEQ ID NO: 172
  source 1..113: mol_type = other DNA

SEQ ID NO: 203 - DNA, 113 bp, synthetic construct; identical to SEQ ID NO: 169
  source 1..113: mol_type = other DNA

SEQ ID NO: 204 - DNA, 100 bp, synthetic construct
  source 1..100: mol_type = other DNA
aacttcaggtgcacgaaaccctggtgtgccctctgatgttttttaggggattctacatctgtagtgagcaattccgtgctcagcaatcaaacgtatgatg

SEQ ID NO: 205 - DNA, 100 bp, synthetic construct
  source 1..100: mol_type = other DNA
caggtgcacgaaaccctggtgtgccctctgatgttttttaggggattctacatctgtagtgagcaattccgtgctcagcaatcaaacgtatgatggcagc

SEQ ID NO: 206 - DNA, 100 bp, synthetic construct
  source 1..100: mol_type = other DNA
gcacgaaaccctggtgtgccctctgatgttttttaggggattctacatctgtagtgagcaattccgtgctcagcaatcaaacgtatgatggcagcattgg

SEQ ID NO: 207 - DNA, 100 bp, synthetic construct
  source 1..100: mol_type = other DNA
aaaccctggtgtgccctctgatgttttttaggggattctacatctgtagtgagcaattccgtgctcagcaatcaaacgtatgatggcagcattgggatac

SEQ ID NO: 208 - DNA, 100 bp, synthetic construct
  source 1..100: mol_type = other DNA
ctggtgtgccctctgatgttttttaggggattctacatctgtagtgagcaattccgtgctcagcaatcaaacgtatgatggcagcattgggatacagtgt

SEQ ID NO: 209 - DNA, 100 bp, synthetic construct
  source 1..100: mol_type = other DNA
gtgccctctgatgttttttaggggattctacatctgtagtgagcaattccgtgctcagcaatcaaacgtatgatggcagcattgggatacagtgtgaaaa

SEQ ID NO: 210 - DNA, 100 bp, synthetic construct
  source 1..100: mol_type = other DNA
aaaccctggtgtgccctctgtccaaattatccccattctacatctgtagtgagcaattccgtgctcagcaatcaaacgtatgatggcagcattgggatac

SEQ ID NO: 211 - DNA, 100 bp, synthetic construct
  source 1..100: mol_type = other DNA
aaaccctggtgtgccctctgatcaaaacatccccattctacatctgtagtgagcaattccgtgctcagcaatcaaacgtatgatggcagcattgggatac

SEQ ID NO: 212 - DNA, 100 bp, synthetic construct
  source 1..100: mol_type = other DNA
aaaccctggtgtgccctctgatgtaaactaccccattctacatctgtagtgagcaattccgtgctcagcaatcaaacgtatgatggcagcattgggatac

SEQ ID NO: 213 - DNA, 100 bp, synthetic construct
  source 1..100: mol_type = other DNA
aaaccctggtgtgccctctgatgtttactaggccattctacatctgtagtgagcaattccgtgctcagcaatcaaacgtatgatggcagcattgggatac

SEQ ID NO: 214 - DNA, 100 bp, synthetic construct; identical to SEQ ID NO: 207
  source 1..100: mol_type = other DNA

SEQ ID NO: 215 - DNA, 100 bp, synthetic construct
  source 1..100: mol_type = other DNA
aaaccctggtgtgccctctgatgtttttaaggggtttctacatctgtagtgagcaattccgtgctcagcaatcaaacgtatgatggcagcattgggatac

SEQ ID NO: 216 - DNA, 100 bp, synthetic construct
  source 1..100: mol_type = other DNA
aaaccctggtgtgccctctgatgtttttatggggtctctacatctgtagtgagcaattccgtgctcagcaatcaaacgtatgatggcagcattgggatac

SEQ ID NO: 217 - DNA, 100 bp, synthetic construct
  source 1..100: mol_type = other DNA
aaaccctggtgtgccctctgatgtttttatcgggtcactacatctgtagtgagcaattccgtgctcagcaatcaaacgtatgatggcagcattgggatac

SEQ ID NO: 218 - DNA, 100 bp, synthetic construct
  source 1..100: mol_type = other DNA
aaaccctggtgtgccctctgatgtttttatccggtcagtacatctgtagtgagcaattccgtgctcagcaatcaaacgtatgatggcagcattgggatac

SEQ ID NO: 219 - DNA, 100 bp, synthetic construct
  source 1..100: mol_type = other DNA
aaaccctggtgtgccctctgtccaaattatccccattctacatctgtagtgagcaattccgtgctcagcaatctttgcaatgaactgtccattgggatac

SEQ ID NO: 220 - DNA, 100 bp, synthetic construct
  source 1..100: mol_type = other DNA
aaaccctggtgtgccctctgtccaaattatccccattctacatctgtagtgagcaattccgtgctcagcaatctttgcaatctactgagcattgggatac

SEQ ID NO: 221 - DNA, 100 bp, synthetic construct
  source 1..100: mol_type = other DNA
aaaccctggtgtgccctctgtccaaattatccccattctacatctgtagtgagcaattccgtgctcagcaatctttgcaccctacgcagcattgggatac

SEQ ID NO: 222 - DNA, 100 bp, synthetic construct
  source 1..100: mol_type = other DNA
aaaccctggtgtgccctctgtccaaattatccccattctacatctgtagtgagcaattccgtgctcagcaatctttggtcccttggcagcattgggatac

SEQ ID NO: 223 - DNA, 100 bp, synthetic construct
  source 1..100: mol_type = other DNA
aaaccctggtgtgccctctgtccaaattatccccattctacatctgtagtgagcaattccgtgctcagcaatcttacgtccgatggcagcattgggatac

SEQ ID NO: 224 - DNA, 100 bp, synthetic construct; identical to SEQ ID NO: 210
  source 1..100: mol_type = other DNA

SEQ ID NO: 225 - DNA, 100 bp, synthetic construct
  source 1..100: mol_type = other DNA
aaaccctggtgtgccctctgtccaaattatccccattctacatctgtagtgagcaattccgtgctcagcaaagaaaccaatgatggcagcattgggatac

SEQ ID NO: 226 - DNA, 100 bp, synthetic construct
  source 1..100: mol_type = other DNA
aaaccctggtgtgccctctgtccaaattatccccattctacatctgtagtgagcaattccgtgctcagctcagaatgcaatgatggcagcattgggatac

SEQ ID NO: 227 - DNA, 90 bp, synthetic construct
  source 1..90: mol_type = other DNA
aaaccctggtgtgccctctgtccaaattatccccattctacatctgtagtgagcaattccgtgctcagctcagaatgcaatgatggcagc

SEQ ID NO: 228 - DNA, 92 bp, synthetic construct
  source 1..92: mol_type = other DNA
aaaccctggtgtgccctctgtccaaattatccccattctacatctgtagtgagcaattccgtgctcagctcagaatgcaatgatggcagcat

SEQ ID NO: 229 - DNA, 94 bp, synthetic construct
  source 1..94: mol_type = other DNA
aaaccctggtgtgccctctgtccaaattatccccattctacatctgtagtgagcaattccgtgctcagctcagaatgcaatgatggcagcattg

SEQ ID NO: 230 - DNA, 96 bp, synthetic construct
  source 1..96: mol_type = other DNA
aaaccctggtgtgccctctgtccaaattatccccattctacatctgtagtgagcaattccgtgctcagctcagaatgcaatgatggcagcattggg

SEQ ID NO: 231 - DNA, 98 bp, synthetic construct
  source 1..98: mol_type = other DNA
aaaccctggtgtgccctctgtccaaattatccccattctacatctgtagtgagcaattccgtgctcagctcagaatgcaatgatggcagcattgggat

SEQ ID NO: 232 - DNA, 90 bp, synthetic construct
  source 1..90: mol_type = other DNA
gtgccctctgtccaaattatccccattctacatctgtagtgagcaattccgtgctcagctcagaatgcaatgatggcagcattgggatac

SEQ ID NO: 233 - DNA, 92 bp, synthetic construct
  source 1..92: mol_type = other DNA
gtgtgccctctgtccaaattatccccattctacatctgtagtgagcaattccgtgctcagctcagaatgcaatgatggcagcattgggatac

SEQ ID NO: 234 - DNA, 94 bp, synthetic construct
  source 1..94: mol_type = other DNA
tggtgtgccctctgtccaaattatccccattctacatctgtagtgagcaattccgtgctcagctcagaatgcaatgatggcagcattgggatac

SEQ ID NO: 235 - DNA, 96 bp, synthetic construct
  source 1..96: mol_type = other DNA
cctggtgtgccctctgtccaaattatccccattctacatctgtagtgagcaattccgtgctcagctcagaatgcaatgatggcagcattgggatac

SEQ ID NO: 236 - DNA, 98 bp, synthetic construct
  source 1..98: mol_type = other DNA
accctggtgtgccctctgtccaaattatccccattctacatctgtagtgagcaattccgtgctcagctcagaatgcaatgatggcagcattgggatac

SEQ ID NO: 237 - DNA, 100 bp, synthetic construct; identical to SEQ ID NO: 226
  source 1..100: mol_type = other DNA

SEQ ID NO: 238 - DNA, 113 bp, synthetic construct; identical to SEQ ID NO: 33
  source 1..113: mol_type = other DNA

SEQ ID NO: 239 - DNA, 113 bp, synthetic construct; identical to SEQ ID NO: 34
  source 1..113: mol_type = other DNA

SEQ ID NO: 240 - DNA, 113 bp, synthetic construct; identical to SEQ ID NO: 30
  source 1..113: mol_type = other DNA

SEQ ID NO: 241 - DNA, 113 bp, synthetic construct; identical to SEQ ID NO: 32
  source 1..113: mol_type = other DNA

SEQ ID NO: 242 - DNA, 113 bp, synthetic construct; identical to SEQ ID NO: 31
  source 1..113: mol_type = other DNA

SEQ ID NO: 243 - DNA, 100 bp, synthetic construct
  source 1..100: mol_type = other DNA
aacttcaggtgcacgaaaccctggtgtgccctctgatgttttttaggggattctaccgctgtgctgggcaatcccgtggtcagcaatcaaacgtatgatg

SEQ ID NO: 244 - DNA, 100 bp, synthetic construct
  source 1..100: mol_type = other DNA
caggtgcacgaaaccctggtgtgccctctgatgttttttaggggattctaccgctgtgctgggcaatcccgtggtcagcaatcaaacgtatgatggcagc

SEQ ID NO: 245 - DNA, 100 bp, synthetic construct
  source 1..100: mol_type = other DNA
gcacgaaaccctggtgtgccctctgatgttttttaggggattctaccgctgtgctgggcaatcccgtggtcagcaatcaaacgtatgatggcagcattgg

SEQ ID NO: 246 - DNA, 100 bp, synthetic construct
  source 1..100: mol_type = other DNA
aaaccctggtgtgccctctgatgttttttaggggattctaccgctgtgctgggcaatcccgtggtcagcaatcaaacgtatgatggcagcattgggatac

SEQ ID NO: 247 - DNA, 100 bp, synthetic construct
  source 1..100: mol_type = other DNA
ctggtgtgccctctgatgttttttaggggattctaccgctgtgctgggcaatcccgtggtcagcaatcaaacgtatgatggcagcattgggatacagtgt

SEQ ID NO: 248 - DNA, 100 bp, synthetic construct
  source 1..100: mol_type = other DNA
gtgccctctgatgttttttaggggattctaccgctgtgctgggcaatcccgtggtcagcaatcaaacgtatgatggcagcattgggatacagtgtgaaaa

SEQ ID NO: 249 - DNA, 113 bp, synthetic construct; identical to SEQ ID NO: 32
  source 1..113: mol_type = other DNA

SEQ ID NO: 250 - DNA, 100 bp, synthetic construct
  source 1..100: mol_type = other DNA
gtgccctctgatgtaaaataccccattctaccgctgtgctgggcaatcccgtggtcagcaatcaaacgtatgatggcagcattgggatacagtgtgaaaa

SEQ ID NO: 251 - DNA, 100 bp, synthetic construct
  source 1..100: mol_type = other DNA
gtgccctctgatgtttaataggccattctaccgctgtgctgggcaatcccgtggtcagcaatcaaacgtatgatggcagcattgggatacagtgtgaaaa

SEQ ID NO: 252 - DNA, 100 bp, synthetic construct; identical to SEQ ID NO: 248
  source 1..100: mol_type = other DNA

SEQ ID NO: 253 - DNA, 100 bp, synthetic construct
  source 1..100: mol_type = other DNA
gtgccctctgatgtttttaaggggtttctaccgctgtgctgggcaatcccgtggtcagcaatcaaacgtatgatggcagcattgggatacagtgtgaaaa

SEQ ID NO: 254 - DNA, 100 bp, synthetic construct
  source 1..100: mol_type = other DNA
gtgccctctgatgtttttatcgggtcactaccgctgtgctgggcaatcccgtggtcagcaatcaaacgtatgatggcagcattgggatacagtgtgaaaa

SEQ ID NO: 255 - DNA, 100 bp, synthetic construct
  source 1..100: mol_type = other DNA
gtgccctctgatgtttttatccggtcagtaccgctgtgctgggcaatcccgtggtcagcaatcaaacgtatgatggcagcattgggatacagtgtgaaaa

SEQ ID NO: 256 - DNA, 100 bp, synthetic construct
  source 1..100: mol_type = other DNA
gtgccctctgatgtttttatcccgtcagaaccgctgtgctgggcaatcccgtggtcagcaatcaaacgtatgatggcagcattgggatacagtgtgaaaa

SEQ ID NO: 257 - DNA, 100 bp, synthetic construct
  source 1..100: mol_type = other DNA
gtgccctctgatgtttaataggccattctaccgctgtgctgggcaatcccgtggtcagcaatctttgcaatgaaccgtccattgggatacagtgtgaaaa

SEQ ID NO: 258 - DNA, 100 bp, synthetic construct
  source 1..100: mol_type = other DNA
gtgccctctgatgtttaataggccattctaccgctgtgctgggcaatcccgtggtcagcaatctttgcaatctaccgagcattgggatacagtgtgaaaa

SEQ ID NO: 259 - DNA, 100 bp, synthetic construct
  source 1..100: mol_type = other DNA
gtgccctctgatgtttaataggccattctaccgctgtgctgggcaatcccgtggtcagcaatctttgcatactacgcagcattgggatacagtgtgaaaa

SEQ ID NO: 260 - DNA, 100 bp, synthetic construct
  source 1..100: mol_type = other DNA
gtgccctctgatgtttaataggccattctaccgctgtgctgggcaatcccgtggtcagcaatctttggttacttggcagcattgggatacagtgtgaaaa

SEQ ID NO: 261 - DNA, 100 bp, synthetic construct
  source 1..100: mol_type = other DNA
gtgccctctgatgtttaataggccattctaccgctgtgctgggcaatcccgtggtcagcaatcttacgttagatggcagcattgggatacagtgtgaaaa

SEQ ID NO: 262 - DNA, 100 bp, synthetic construct; identical to SEQ ID NO: 251
  source 1..100: mol_type = other DNA

SEQ ID NO: 263 - DNA, 100 bp, synthetic construct
  source 1..100: mol_type = other DNA
gtgccctctgatgtttaataggccattctaccgctgtgctgggcaatcccgtggtcagcaaagaaaccaatgatggcagcattgggatacagtgtgaaaa

SEQ ID NO: 264 - DNA, 100 bp, synthetic construct
  source 1..100: mol_type = other DNA
gtgccctctgatgtttaataggccattctaccgctgtgctgggcaatcccgtggtcagctcagaatgcaatgatggcagcattgggatacagtgtgaaaa

SEQ ID NO: 265 - DNA, 100 bp, synthetic construct
  source 1..100: mol_type = other DNA
gtgccctctgatgtttaataggccattctaccgctgtgctgggcaatcccgtggtcaggtcagattgcaatgatggcagcattgggatacagtgtgaaaa

SEQ ID NO: 266 - DNA, 100 bp, synthetic construct
  source 1..100: mol_type = other DNA
gtgccctctgatgtttaataggccattctaccgctgtgctgggcaatcccgtggtctcgtcactttgcaatgatggcagcattgggatacagtgtgaaaa

SEQ ID NO: 267 - DNA, 113 bp, synthetic construct; identical to SEQ ID NO: 28
  source 1..113: mol_type = other DNA

SEQ ID NO: 268 - DNA, 113 bp, synthetic construct; identical to SEQ ID NO: 29
  source 1..113: mol_type = other DNA

SEQ ID NO: 269 - DNA, 113 bp, synthetic construct; identical to SEQ ID NO: 12
  source 1..113: mol_type = other DNA

SEQ ID NO: 270 - DNA, 113 bp, synthetic construct; identical to SEQ ID NO: 13
  source 1..113: mol_type = other DNA

SEQ ID NO: 271 - DNA, 113 bp, synthetic construct; identical to SEQ ID NO: 10
  source 1..113: mol_type = other DNA

SEQ ID NO: 272 - DNA, 113 bp, synthetic construct; identical to SEQ ID NO: 11
  source 1..113: mol_type = other DNA

SEQ ID NO: 273 - DNA, 113 bp, synthetic construct; identical to SEQ ID NO: 8
  source 1..113: mol_type = other DNA

SEQ ID NO: 274 - DNA, 113 bp, synthetic construct; identical to SEQ ID NO: 9
  source 1..113: mol_type = other DNA

SEQ ID NO: 275 - DNA, 113 bp, synthetic construct; identical to SEQ ID NO: 2
  source 1..113: mol_type = other DNA

SEQ ID NO: 276 - DNA, 113 bp, synthetic construct; identical to SEQ ID NO: 3
  source 1..113: mol_type = other DNA

SEQ ID NO: 277 - DNA, 113 bp, synthetic construct; identical to SEQ ID NO: 6
  source 1..113: mol_type = other DNA

SEQ ID NO: 278 - DNA, 113 bp, synthetic construct; identical to SEQ ID NO: 7
  source 1..113: mol_type = other DNA

SEQ ID NO: 279 - DNA, 113 bp, synthetic construct; identical to SEQ ID NO: 20
  source 1..113: mol_type = other DNA

SEQ ID NO: 280 - DNA, 113 bp, synthetic construct; identical to SEQ ID NO: 24
  source 1..113: mol_type = other DNA

SEQ ID NO: 281 - DNA, 113 bp, synthetic construct; identical to SEQ ID NO: 26
  source 1..113: mol_type = other DNA

SEQ ID NO: 282 - DNA, 113 bp, synthetic construct; identical to SEQ ID NO: 25
  source 1..113: mol_type = other DNA

SEQ ID NO: 283 - DNA, 113 bp, synthetic construct; identical to SEQ ID NO: 22
  source 1..113: mol_type = other DNA

SEQ ID NO: 284 - DNA, 113 bp, synthetic construct
  source 1..113: mol_type = other DNA
ccctggtgtgccctctgatgttctttaggggattctacagtaggactgagcactgccgagctgggcaatctttgcatactacgcagcattgggatacagtgtgaagagcagca

SEQ ID NO: 285 - DNA, 100 bp, synthetic construct
  source 1..100: mol_type = other DNA
aacttcaggtgcacgaaaccctggtgtgccctctgatgtttttatggggtgtctacagtaggactgagcactgccgagctgggcaatcttgagtcggatg

SEQ ID NO: 286 - DNA, 100 bp, synthetic construct
  source 1..100: mol_type = other DNA
caggtgcacgaaaccctggtgtgccctctgatgtttttatggggtgtctacagtaggactgagcactgccgagctgggcaatcttgagtcggatggcagc

SEQ ID NO: 287 - DNA, 100 bp, synthetic construct
  source 1..100: mol_type = other DNA
gcacgaaaccctggtgtgccctctgatgtttttatggggtgtctacagtaggactgagcactgccgagctgggcaatcttgagtcggatggcagcattgg

SEQ ID NO: 288 - DNA, 100 bp, synthetic construct
  source 1..100: mol_type = other DNA
aaaccctggtgtgccctctgatgtttttatggggtgtctacagtaggactgagcactgccgagctgggcaatcttgagtcggatggcagcattgggatac

SEQ ID NO: 289 - DNA, 100 bp, synthetic construct
  source 1..100: mol_type = other DNA
ctggtgtgccctctgatgtttttatggggtgtctacagtaggactgagcactgccgagctgggcaatcttgagtcggatggcagcattgggatacagtgt

SEQ ID NO: 290 - DNA, 100 bp, synthetic construct
  source 1..100: mol_type = other DNA
gtgccctctgatgtttttatggggtgtctacagtaggactgagcactgccgagctgggcaatcttgagtcggatggcagcattgggatacagtgtgaaaa

SEQ ID NO: 291 - DNA, 100 bp, synthetic construct
  source 1..100: mol_type = other DNA
ctggtgtgccctctgtaccaattatccccattctacagtaggactgagcactgccgagctgggcaatcttgagtcggatggcagcattgggatacagtgt

SEQ ID NO: 292 - DNA, 100 bp, synthetic construct
  source 1..100: mol_type = other DNA
ctggtgtgccctctgatccaagcatccccattctacagtaggactgagcactgccgagctgggcaatcttgagtcggatggcagcattgggatacagtgt

SEQ ID NO: 293 - DNA, 100 bp, synthetic construct
  source 1..100: mol_type = other DNA
ctggtgtgccctctgatgtaagctaccccattctacagtaggactgagcactgccgagctgggcaatcttgagtcggatggcagcattgggatacagtgt

SEQ ID NO: 294 - DNA, 100 bp, synthetic construct
  source 1..100: mol_type = other DNA
ctggtgtgccctctgatgtttgctaggccattctacagtaggactgagcactgccgagctgggcaatcttgagtcggatggcagcattgggatacagtgt

SEQ ID NO: 295 - DNA, 100 bp, synthetic construct
  source 1..100: mol_type = other DNA
ctggtgtgccctctgatgttttttaggggattctacagtaggactgagcactgccgagctgggcaatcttgagtcggatggcagcattgggatacagtgt

SEQ ID NO: 296 - DNA, 100 bp, synthetic construct; identical to SEQ ID NO: 289
  source 1..100: mol_type = other DNA

SEQ ID NO: 297 - DNA, 100 bp, synthetic construct
  source 1..100: mol_type = other DNA
ctggtgtgccctctgatgtttttatccggtgagtacagtaggactgagcactgccgagctgggcaatcttgagtcggatggcagcattgggatacagtgt

SEQ ID NO: 298 - DNA, 100 bp, synthetic construct
  source 1..100: mol_type = other DNA
ctggtgtgccctctgatgtttttatcccctgagatcagtaggactgagcactgccgagctgggcaatcttgagtcggatggcagcattgggatacagtgt

SEQ ID NO: 299 - DNA, 100 bp, synthetic construct
  source 1..100: mol_type = other DNA
ctggtgtgccctctgatgtttttatccccatagatgtgtaggactgagcactgccgagctgggcaatcttgagtcggatggcagcattgggatacagtgt

SEQ ID NO: 300 - DNA, 100 bp, synthetic construct
  source 1..100: mol_type = other DNA
ctggtgtgccctctgatccaagcatccccattctacagtaggactgagcactgccgagctgggcaatctttgcaatgatggcaggtgaccgatacagtgt

SEQ ID NO: 301 - DNA, 100 bp, synthetic construct
  source 1..100: mol_type = other DNA
ctggtgtgccctctgatccaagcatccccattctacagtaggactgagcactgccgagctgggcaatctttgcaatgatgggtcgtgtgggatacagtgt

SEQ ID NO: 302 - DNA, 100 bp, synthetic construct
  source 1..100: mol_type = other DNA
ctggtgtgccctctgatccaagcatccccattctacagtaggactgagcactgccgagctgggcaatctttgcaatgaaccgtccattgggatacagtgt

SEQ ID NO: 303 - DNA, 100 bp, synthetic construct
  source 1..100: mol_type = other DNA
ctggtgtgccctctgatccaagcatccccattctacagtaggactgagcactgccgagctgggcaatctttgcaatctaccgagcattgggatacagtgt

SEQ ID NO: 304 - DNA, 100 bp, synthetic construct
  source 1..100: mol_type = other DNA
ctggtgtgccctctgatccaagcatccccattctacagtaggactgagcactgccgagctgggcaatctttgcacgctacgcagcattgggatacagtgt

SEQ ID NO: 305 - DNA, 100 bp, synthetic construct
  source 1..100: mol_type = other DNA
ctggtgtgccctctgatccaagcatccccattctacagtaggactgagcactgccgagctgggcaatctttggtcgcttggcagcattgggatacagtgt

SEQ ID NO: 306 - DNA, 100 bp, synthetic construct; identical to SEQ ID NO: 292
  source 1..100: mol_type = other DNA

SEQ ID NO: 307 - DNA, 100 bp, synthetic construct
  source 1..100: mol_type = other DNA
ctggtgtgccctctgatccaagcatccccattctacagtaggactgagcactgccgagctgggcaatgaagagaatgatggcagcattgggatacagtgt

SEQ ID NO: 308 - DNA, 90 bp, synthetic construct
  source 1..90: mol_type = other DNA
ctggtgtgccctctgatccaagcatccccattctacagtaggactgagcactgccgagctgggcaatcttgagtcggatggcagcattgg

SEQ ID NO: 309 - DNA, 92 bp, synthetic construct
  source 1..92: mol_type = other DNA
ctggtgtgccctctgatccaagcatccccattctacagtaggactgagcactgccgagctgggcaatcttgagtcggatggcagcattggga

SEQ ID NO: 310 - DNA, 94 bp, synthetic construct
  source 1..94: mol_type = other DNA
ctggtgtgccctctgatccaagcatccccattctacagtaggactgagcactgccgagctgggcaatcttgagtcggatggcagcattgggata

SEQ ID NO: 311 - DNA, 96 bp, synthetic construct
  source 1..96: mol_type = other DNA
ctggtgtgccctctgatccaagcatccccattctacagtaggactgagcactgccgagctgggcaatcttgagtcggatggcagcattgggataca

SEQ ID NO: 312 - DNA, 98 bp, synthetic construct
  source 1..98: mol_type = other DNA
ctggtgtgccctctgatccaagcatccccattctacagtaggactgagcactgccgagctgggcaatcttgagtcggatggcagcattgggatacagt

SEQ ID NO: 313 - DNA, 90 bp, synthetic construct
  source 1..90: mol_type = other DNA
ctctgatccaagcatccccattctacagtaggactgagcactgccgagctgggcaatcttgagtcggatggcagcattgggatacagtgt

SEQ ID NO: 314 - DNA, 92 bp, synthetic construct
  source 1..92: mol_type = other DNA
ccctctgatccaagcatccccattctacagtaggactgagcactgccgagctgggcaatcttgagtcggatggcagcattgggatacagtgt

SEQ ID NO: 315 - DNA, 94 bp, synthetic construct
  source 1..94: mol_type = other DNA
tgccctctgatccaagcatccccattctacagtaggactgagcactgccgagctgggcaatcttgagtcggatggcagcattgggatacagtgt

SEQ ID NO: 316 - DNA, 96 bp, synthetic construct
  source 1..96: mol_type = other DNA
tgtgccctctgatccaagcatccccattctacagtaggactgagcactgccgagctgggcaatcttgagtcggatggcagcattgggatacagtgt

SEQ ID NO: 317 - DNA, 98 bp, synthetic construct
  source 1..98: mol_type = other DNA
ggtgtgccctctgatccaagcatccccattctacagtaggactgagcactgccgagctgggcaatcttgagtcggatggcagcattgggatacagtgt

SEQ ID NO: 318 - DNA, 100 bp, synthetic construct; identical to SEQ ID NO: 292
  source 1..100: mol_type = other DNA

SEQ ID NO: 319 - DNA, 113 bp, synthetic construct; identical to SEQ ID NO: 43
  source 1..113: mol_type = other DNA

SEQ ID NO: 320 - DNA, 113 bp, synthetic construct; identical to SEQ ID NO: 44
  source 1..113: mol_type = other DNA

SEQ ID NO: 321 - DNA, 113 bp, synthetic construct; identical to SEQ ID NO: 45
  source 1..113: mol_type = other DNA

SEQ ID NO: 322 - DNA, 113 bp, synthetic construct; identical to SEQ ID NO: 46
  source 1..113: mol_type = other DNA

SEQ ID NO: 323 - DNA, 113 bp, synthetic construct; identical to SEQ ID NO: 47
  source 1..113: mol_type = other DNA

SEQ ID NO: 324 - DNA, 113 bp, synthetic construct; identical to SEQ ID NO: 48
  source 1..113: mol_type = other DNA

SEQ ID NO: 325 - DNA, 113 bp, synthetic construct; identical to SEQ ID NO: 49
  source 1..113: mol_type = other DNA

SEQ ID NO: 326 - DNA, 113 bp, synthetic construct; identical to SEQ ID NO: 51
  source 1..113: mol_type = other DNA

SEQ ID NO: 327 - DNA, 100 bp, synthetic construct
  source 1..100: mol_type = other DNA
aacttcaggtgcacgaaaccctggtgtgccctctgatgtttttatgaggcgtctacagcagttcggaggaatcccgaggtcagcaagactaacaatgatg

SEQ ID NO: 328 - DNA, 100 bp, synthetic construct
  source 1..100: mol_type = other DNA
caggtgcacgaaaccctggtgtgccctctgatgtttttatgaggcgtctacagcagttcggaggaatcccgaggtcagcaagactaacaatgatggcagc

SEQ ID NO: 329 - DNA, 100 bp, synthetic construct
  source 1..100: mol_type = other DNA
gcacgaaaccctggtgtgccctctgatgtttttatgaggcgtctacagcagttcggaggaatcccgaggtcagcaagactaacaatgatggcagcattgg

SEQ ID NO: 330 - DNA, 100 bp, synthetic construct
  source 1..100: mol_type = other DNA
aaaccctggtgtgccctctgatgtttttatgaggcgtctacagcagttcggaggaatcccgaggtcagcaagactaacaatgatggcagcattgggatac

SEQ ID NO: 331 - DNA, 100 bp, synthetic construct
  source 1..100: mol_type = other DNA
ctggtgtgccctctgatgtttttatgaggcgtctacagcagttcggaggaatcccgaggtcagcaagactaacaatgatggcagcattgggatacagtgt

SEQ ID NO: 332 - DNA, 100 bp, synthetic construct
  source 1..100: mol_type = other DNA
gtgccctctgatgtttttatgaggcgtctacagcagttcggaggaatcccgaggtcagcaagactaacaatgatggcagcattgggatacagtgtgaaaa

SEQ ID NO: 333 - DNA, 100 bp, synthetic construct
  source 1..100: mol_type = other DNA
gtgccctctgtacaaattatccccattctacagcagttcggaggaatcccgaggtcagcaagactaacaatgatggcagcattgggatacagtgtgaaaa

SEQ ID NO: 334 - DNA, 100 bp, synthetic construct
  source 1..100: mol_type = other DNA
gtgccctctgatcaaagcatccccattctacagcagttcggaggaatcccgaggtcagcaagactaacaatgatggcagcattgggatacagtgtgaaaa

SEQ ID NO: 335 - DNA, 100 bp, synthetic construct
  source 1..100: mol_type = other DNA
gtgccctctgatgtaagctaccccattctacagcagttcggaggaatcccgaggtcagcaagactaacaatgatggcagcattgggatacagtgtgaaaa

SEQ ID NO: 336 - DNA, 100 bp, synthetic construct
  source 1..100: mol_type = other DNA
gtgccctctgatgtttgctagaccattctacagcagttcggaggaatcccgaggtcagcaagactaacaatgatggcagcattgggatacagtgtgaaaa

SEQ ID NO: 337 - DNA, 100 bp, synthetic construct
  source 1..100: mol_type = other DNA
gtgccctctgatgttttttagaggattctacagcagttcggaggaatcccgaggtcagcaagactaacaatgatggcagcattgggatacagtgtgaaaa

SEQ ID NO: 338 - DNA, 100 bp, synthetic construct; identical to SEQ ID NO: 332
  source 1..100: mol_type = other DNA

SEQ ID NO: 339 - DNA, 100 bp, synthetic construct
  source 1..100: mol_type = other DNA
gtgccctctgatgtttttatccggcgagtacagcagttcggaggaatcccgaggtcagcaagactaacaatgatggcagcattgggatacagtgtgaaaa

SEQ ID NO: 340 - DNA, 100 bp, synthetic construct
  source 1..100: mol_type = other DNA
gtgccctctgatgtttttatcccccgagatcagcagttcggaggaatcccgaggtcagcaagactaacaatgatggcagcattgggatacagtgtgaaaa

SEQ ID NO: 341 - DNA, 100 bp, synthetic construct
  source 1..100: mol_type = other DNA
gtgccctctgatgtttttatccccatagatgtgcagttcggaggaatcccgaggtcagcaagactaacaatgatggcagcattgggatacagtgtgaaaa

SEQ ID NO: 342 - DNA, 100 bp, synthetic construct
  source 1..100: mol_type = other DNA
gtgccctctgatgtttttatccggcgagtacagcagttcggaggaatcccgaggtcagcaatctttgcaatgaaccgtccattgggatacagtgtgaaaa

SEQ ID NO: 343 - DNA, 100 bp, synthetic construct
  source 1..100: mol_type = other DNA
gtgccctctgatgtttttatccggcgagtacagcagttcggaggaatcccgaggtcagcaatctttgcaatctaccgagcattgggatacagtgtgaaaa

SEQ ID NO: 344 - DNA, 100 bp, synthetic construct
  source 1..100: mol_type = other DNA
gtgccctctgatgtttttatccggcgagtacagcagttcggaggaatcccgaggtcagcaatctttgcatactacgcagcattgggatacagtgtgaaaa

SEQ ID NO: 345 - DNA, 100 bp, synthetic construct
  source 1..100: mol_type = other DNA
gtgccctctgatgtttttatccggcgagtacagcagttcggaggaatcccgaggtcagcaatctttggttacttggcagcattgggatacagtgtgaaaa

SEQ ID NO: 346 - DNA, 100 bp, synthetic construct
  source 1..100: mol_type = other DNA
gtgccctctgatgtttttatccggcgagtacagcagttcggaggaatcccgaggtcagcaatcttaagttagatggcagcattgggatacagtgtgaaaa

SEQ ID NO: 347 - DNA, 100 bp, synthetic construct
  source 1..100: mol_type = other DNA
gtgccctctgatgtttttatccggcgagtacagcagttcggaggaatcccgaggtcagcaatcctaagtatgatggcagcattgggatacagtgtgaaaa

SEQ ID NO: 348 - DNA, 100 bp, synthetic construct; identical to SEQ ID NO: 339
  source 1..100: mol_type = other DNA

SEQ ID NO: 349 - DNA, 100 bp, synthetic construct
  source 1..100: mol_type = other DNA
gtgccctctgatgtttttatccggcgagtacagcagttcggaggaatcccgaggtcaggttgacttgcaatgatggcagcattgggatacagtgtgaaaa

SEQ ID NO: 350 - DNA, 100 bp, synthetic construct
  source 1..100: mol_type = other DNA
gtgccctctgatgtttttatccggcgagtacagcagttcggaggaatcccgaggtctcgttgctttgcaatgatggcagcattgggatacagtgtgaaaa

SEQ ID NO: 351 - DNA, 90 bp, synthetic construct
  source 1..90: mol_type = other DNA
gtgccctctgatgtttttatccggcgagtacagcagttcggaggaatcccgaggtcagcaatcctaagtatgatggcagcattgggatac

SEQ ID NO: 352 - DNA, 92 bp, synthetic construct
  source 1..92: mol_type = other DNA
gtgccctctgatgtttttatccggcgagtacagcagttcggaggaatcccgaggtcagcaatcctaagtatgatggcagcattgggatacag

SEQ ID NO: 353 - DNA, 94 bp, synthetic construct
  source 1..94: mol_type = other DNA
gtgccctctgatgtttttatccggcgagtacagcagttcggaggaatcccgaggtcagcaatcctaagtatgatggcagcattgggatacagtg

SEQ ID NO: 354 - DNA, 96 bp, synthetic construct
  source 1..96: mol_type = other DNA
gtgccctctgatgtttttatccggcgagtacagcagttcggaggaatcccgaggtcagcaatcctaagtatgatggcagcattgggatacagtgtg

SEQ ID NO: 355 - DNA, 98 bp, synthetic construct
  source 1..98: mol_type = other DNA
gtgccctctgatgtttttatccggcgagtacagcagttcggaggaatcccgaggtcagcaatcctaagtatgatggcagcattgggatacagtgtgaa

SEQ ID NO: 356 - DNA, 90 bp, synthetic construct
  source 1..90: mol_type = other DNA
atgtttttatccggcgagtacagcagttcggaggaatcccgaggtcagcaatcctaagtatgatggcagcattgggatacagtgtgaaaa

SEQ ID NO: 357 - DNA, 92 bp, synthetic construct
  source 1..92: mol_type = other DNA
tgatgtttttatccggcgagtacagcagttcggaggaatcccgaggtcagcaatcctaagtatgatggcagcattgggatacagtgtgaaaa

SEQ ID NO: 358 - DNA, 94 bp, synthetic construct
  source 1..94: mol_type = other DNA
tctgatgtttttatccggcgagtacagcagttcggaggaatcccgaggtcagcaatcctaagtatgatggcagcattgggatacagtgtgaaaa

SEQ ID NO: 359 - DNA, 96 bp, synthetic construct
  source 1..96: mol_type = other DNA
cctctgatgtttttatccggcgagtacagcagttcggaggaatcccgaggtcagcaatcctaagtatgatggcagcattgggatacagtgtgaaaa

SEQ ID NO: 360 - DNA, 98 bp, synthetic construct
  source 1..98: mol_type = other DNA
gccctctgatgtttttatccggcgagtacagcagttcggaggaatcccgaggtcagcaatcctaagtatgatggcagcattgggatacagtgtgaaaa

SEQ ID NO: 361 - DNA, 100 bp, synthetic construct; identical to SEQ ID NO: 347
  source 1..100: mol_type = other DNA

SEQ ID NO: 362 - DNA, 113 bp, synthetic construct; identical to SEQ ID NO: 37
  source 1..113: mol_type = other DNA

SEQ ID NO: 363 - DNA, 113 bp, synthetic construct; identical to SEQ ID NO: 36
  source 1..113: mol_type = other DNA

SEQ ID NO: 364 - DNA, 113 bp, synthetic construct; identical to SEQ ID NO: 35
  source 1..113: mol_type = other DNA

SEQ ID NO: 365 - DNA, 113 bp, synthetic construct; identical to SEQ ID NO: 40
  source 1..113: mol_type = other DNA

SEQ ID NO: 366 - DNA, 113 bp, synthetic construct; identical to SEQ ID NO: 39
  source 1..113: mol_type = other DNA

SEQ ID NO: 367 - DNA, 113 bp, synthetic construct; identical to SEQ ID NO: 38
  source 1..113: mol_type = other DNA

SEQ ID NO: 368 - DNA, 100 bp, synthetic construct
  source 1..100: mol_type = other DNA
caggtgcacgaaaccctggtgtgccctctgatgtttttctggtgattctacaacagtactgagctatcccgaattcaacaatctttgcaataagcgaagc

SEQ ID NO: 369 - DNA, 100 bp, synthetic construct
  source 1..100: mol_type = other DNA
gcacgaaaccctggtgtgccctctgatgtttttctggtgattctacaacagtactgagctatcccgaattcaacaatctttgcaataagcgaagcattgg

SEQ ID NO: 370 - DNA, 100 bp, synthetic construct
  source 1..100: mol_type = other DNA
aaaccctggtgtgccctctgatgtttttctggtgattctacaacagtactgagctatcccgaattcaacaatctttgcaataagcgaagcattgggatac

SEQ ID NO: 371 - DNA, 100 bp, synthetic construct
  source 1..100: mol_type = other DNA
ctggtgtgccctctgatgtttttctggtgattctacaacagtactgagctatcccgaattcaacaatctttgcaataagcgaagcattgggatacagtgt

SEQ ID NO: 372 - DNA, 100 bp, synthetic construct
  source 1..100: mol_type = other DNA
gtgccctctgatgtttttctggtgattctacaacagtactgagctatcccgaattcaacaatctttgcaataagcgaagcattgggatacagtgtgaaaa

SEQ ID NO: 373 - DNA, 113 bp, synthetic construct; identical to SEQ ID NO: 38
  source 1..113: mol_type = other DNA

SEQ ID NO: 374 - DNA, 100 bp, synthetic construct
  source 1..100: mol_type = other DNA
gcacgaaaccctggtgtgccctctgtacaaattatccccattctacaacagtactgagctatcccgaattcaacaatctttgcaataagcgaagcattgg

SEQ ID NO: 375 - DNA, 100 bp, synthetic construct
  source 1..100: mol_type = other DNA
gcacgaaaccctggtgtgccctctgatcaaaaaatccccattctacaacagtactgagctatcccgaattcaacaatctttgcaataagcgaagcattgg

SEQ ID NO: 376 - DNA, 100 bp, synthetic construct
  source 1..100: mol_type = other DNA
gcacgaaaccctggtgtgccctctgatgtaaaatgccccattctacaacagtactgagctatcccgaattcaacaatctttgcaataagcgaagcattgg

SEQ ID NO: 377 - DNA, 100 bp, synthetic construct
  source 1..100: mol_type = other DNA
gcacgaaaccctggtgtgccctctgatgtttaatgggccattctacaacagtactgagctatcccgaattcaacaatctttgcaataagcgaagcattgg

SEQ ID NO: 378 - DNA, 100 bp, synthetic construct
  source 1..100: mol_type = other DNA
gcacgaaaccctggtgtgccctctgatgttttttgggtgattctacaacagtactgagctatcccgaattcaacaatctttgcaataagcgaagcattgg

SEQ ID NO: 379 - DNA, 100 bp, synthetic construct; identical to SEQ ID NO: 369
  source 1..100: mol_type = other DNA

SEQ ID NO: 380 - DNA, 100 bp, synthetic construct
  source 1..100: mol_type = other DNA
gcacgaaaccctggtgtgccctctgatgtttttatggtgtatctacaacagtactgagctatcccgaattcaacaatctttgcaataagcgaagcattgg

SEQ ID NO: 381 - DNA, 100 bp, synthetic construct
  source 1..100: mol_type = other DNA
gcacgaaaccctggtgtgccctctgatgtttttatccggtaggtacaacagtactgagctatcccgaattcaacaatctttgcaataagcgaagcattgg

SEQ ID NO: 382 - DNA, 100 bp, synthetic construct
  source 1..100: mol_type = other DNA
gcacgaaaccctggtgtgccctctgatgtttttatcccctaggatcaacagtactgagctatcccgaattcaacaatctttgcaataagcgaagcattgg

SEQ ID NO: 383 - DNA, 100 bp, synthetic construct
  source 1..100: mol_type = other DNA
gcacgaaaccctggtgtgccctctgatgttttttgggtgattctacaacagtactgagctatcccgaattcaacaatctttgcaatgatcaatcaattgg

SEQ ID NO: 384 - DNA, 100 bp, synthetic construct; identical to SEQ ID NO: 369
  source 1..100: mol_type = other DNA

SEQ ID NO: 385 - DNA, 100 bp, synthetic construct
  source 1..100: mol_type = other DNA
gcacgaaaccctggtgtgccctctgatgttttttgggtgattctacaacagtactgagctatcccgaattcaacaatctttgcacaaagcgcagcattgg

SEQ ID NO: 386 - DNA, 100 bp, synthetic construct
  source 1..100: mol_type = other DNA
gcacgaaaccctggtgtgccctctgatgttttttgggtgattctacaacagtactgagctatcccgaattcaacaatctttggtcaattggcagcattgg

SEQ ID NO: 387 - DNA, 100 bp, synthetic construct
  source 1..100: mol_type = other DNA
gcacgaaaccctggtgtgccctctgatgttttttgggtgattctacaacagtactgagctatcccgaattcaacaatcttaagtcagatggcagcattgg

SEQ ID NO: 388 - DNA, 100 bp, synthetic construct
  source 1..100: mol_type = other DNA
gcacgaaaccctggtgtgccctctgatgttttttgggtgattctacaacagtactgagctatcccgaattcaacaatcaaaagtatgatggcagcattgg

SEQ ID NO: 389 - DNA, 113 bp, synthetic construct; identical to SEQ ID NO: 27
  source 1..113: mol_type = other DNA

SEQ ID NO: 390 - DNA, 113 bp, synthetic construct; identical to SEQ ID NO: 19
  source 1..113: mol_type = other DNA

SEQ ID NO: 391 - DNA, 113 bp, synthetic construct; identical to SEQ ID NO: 18
  source 1..113: mol_type = other DNA

SEQ ID NO: 392 - DNA, 113 bp, synthetic construct; identical to SEQ ID NO: 17
  source 1..113: mol_type = other DNA

SEQ ID NO: 393 - DNA, 113 bp, synthetic construct; identical to SEQ ID NO: 16
  source 1..113: mol_type = other DNA

SEQ ID NO: 394 - DNA, 113 bp, synthetic construct; identical to SEQ ID NO: 15
  source 1..113: mol_type = other DNA

SEQ ID NO: 395 - DNA, 113 bp, synthetic construct; identical to SEQ ID NO: 14
  source 1..113: mol_type = other DNA

SEQ ID NO: 396 - DNA, 393 bp, synthetic construct
  source 1..393: mol_type = other DNA
taaggaccagcttctttgggagagaacagacgcaggggcgggagggaaaaagggagaggcagacgtcacttcctcttggcgactctggcagcagattggtcggttgagtggcagaaaggcagacggggactgggcaaggcactgtcggtgacatcacggacagggcgacttctatgtagatgaggcagcgcagaggctgctgcttcgccacttgctgcttcgccacgaagggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagagcccgaagatctc

SEQ ID NO: 397 - DNA, 11 bp, synthetic construct
  source 1..11: mol_type = other DNA
aatttttggag

SEQ ID NO: 398 - DNA, 100 bp, synthetic construct
  source 1..100: mol_type = other DNA
gccttcgggcatggcggacttgaagaagtcgtgctgcttcatgtggtcggggtagcggctgaagcactgcactccgtaggtcagggtcgtcacgagggtc

SEQ ID NO: 399 - DNA, 100 bp, synthetic construct
  source 1..100: mol_type = other DNA
aagtcgtgctgcttcatgtggtcggggtagcggctgaagcactgcactccgtaggtcagggtcgtcacgagggtcggccagggcacgggcagcttgccgg

SEQ ID NO: 400 - DNA, 100 bp, synthetic construct
  source 1..100: mol_type = other DNA
ggtagcggctgaagcactgcactccgtaggtcagggtcgtcacgagggtcggccagggcacgggcagcttgccggtggtgcagatgaacttcagggtcag

SEQ ID NO: 401 - DNA, 100 bp, synthetic construct
  source 1..100: mol_type = other DNA
gccttcgggcatggcggacttgaagaagtcgtgctgcttcatgtggtcggggtagcgcctgaagcactggacagccctcgtctgggtcgtcacgagggtc

SEQ ID NO: 402 - DNA, 100 bp, synthetic construct
  source 1..100: mol_type = other DNA
gccttcgggcatggcggacttgaagaagtcgtgctgcttcatgtggtcggggtagcggctgatcaactccacagccaaggtcagggtcgtcacgagggtc

SEQ ID NO: 403 - DNA, 100 bp, synthetic construct
  source 1..100: mol_type = other DNA
gccttcgggcatggcggacttgaagaagtcgtgctgcttcatgtggtcggggtagcggctgtagcactgccctcccatccacagggtcgtcacgagggtc

SEQ ID NO: 404 - DNA, 100 bp, synthetic construct
  source 1..100: mol_type = other DNA
gccttcgggcatggcggacttgaagaagtcgtgctgcttcatgtggtcggggtagcggctgaaggactcgtctcccaaggtctgggtcgtcacgagggtc

SEQ ID NO: 405 - DNA, 100 bp, synthetic construct
  source 1..100: mol_type = other DNA
aagtcgtgctgcttcatgtggtcggggtagcgcctgaagcactggacagccctcgtctgggtcgtcacgagggtcggccagggcacgggcagcttgccgg

SEQ ID NO: 406 - DNA, 100 bp, synthetic construct
  source 1..100: mol_type = other DNA
aagtcgtgctgcttcatgtggtcggggtagcggctgatcaactccacagccaaggtcagggtcgtcacgagggtcggccagggcacgggcagcttgccgg

SEQ ID NO: 407 - DNA, 100 bp, synthetic construct
  source 1..100: mol_type = other DNA
aagtcgtgctgcttcatgtggtcggggtagcggctgtagcactgccctcccatccacagggtcgtcacgagggtcggccagggcacgggcagcttgccgg

SEQ ID NO: 408 - DNA, 100 bp, synthetic construct
  source 1..100: mol_type = other DNA
aagtcgtgctgcttcatgtggtcggggtagcggctgaaggactcgtctcccaaggtctgggtcgtcacgagggtcggccagggcacgggcagcttgccgg

SEQ ID NO: 409 - DNA, 100 bp, synthetic construct
  source 1..100: mol_type = other DNA
ggtagcggctgaacgtgaccactccgtaggtcagggtcgtcacgagggtcggcctcccgtcgggcagcttgccggtggtgcagatgaacttcagggtcag

SEQ ID NO: 410 - DNA, 100 bp, synthetic construct
  source 1..100: mol_type = other DNA
ggtagcggctgaaggtgacgactccgtaggtcagggtcgtcacgagggtcggcctcccgtcgggcagcttgccggtggtgcagatgaacttcagggtcag

SEQ ID NO: 411 - DNA, 100 bp, synthetic construct
  source 1..100: mol_type = other DNA
gccttcgggcatggcggacttgaagaagtcgtgctgcttgtacacgtcggggtagcggctgaagcactgcactccgtaggagtccctcgtcacgagggtc

SEQ ID NO: 412 - DNA, 100 bp, synthetic construct
  source 1..100: mol_type = other DNA
aagtcgtgctgcttgtacacgtcggggtagcggctgaagcactgcactccgtaggagtccctcgtcacgagggtcggccagggcacgggcagcttgccgg

SEQ ID NO: 413 - DNA, 100 bp, synthetic construct
  source 1..100: mol_type = other DNA
gccttcgggcatggcggacttgaagaagtcgtgctgcttgtacacgtcggggtagcggctgaagcactgcactccgtagtaatccgtcgtcacgagggtc

SEQ ID NO: 414 - RNA, 98 nt, synthetic construct
  source 1..98: mol_type = other RNA
ctggtgtgccctctgatccaagcatccccattctacagtaggactgagcactgccgagctgggcaatcttgagtcggatggcagcattgggatacagt

SEQ ID NO: 415 - RNA, 274 nt, synthetic construct
  source 1..274: mol_type = other RNA
ggccatcagtcgccggtcccaagcccggataaaatgggagggggcgggaaaccgcctaaccatgccgactgatggcagataatctggtgtgccctctgatccaagcatccccattctacagtaggactgagcactgccgagctgggcaatcttgagtcggatggcagcattgggatacagtatatactgccatcagtcggcgtggactgtagaacactgccaatgccggtcccaagcccggataaaagtggagggtacagtccacgcttttttt

SEQ ID NO: 416 - RNA, 96 nt, synthetic construct
  source 1..96: mol_type = other RNA
gtgccctctgatgtttttatccggcgagtacagcagttcggaggaatcccgaggtcagcaatcctaagtatgatggcagcattgggatacagtgtg

SEQ ID NO: 417 - RNA, 272 nt, synthetic construct
  source 1..272: mol_type = other RNA
ggccatcagtcgccggtcccaagcccggataaaatgggagggggcgggaaaccgcctaaccatgccgactgatggcagataatgtgccctctgatgtttttatccggcgagtacagcagttcggaggaatcccgaggtcagcaatcctaagtatgatggcagcattgggatacagtgtgatatactgccatcagtcggcgtggactgtagaacactgccaatgccggtcccaagcccggataaaagtggagggtacagtccacgcttttttt

SEQ ID NO: 418 - RNA, 374 nt, synthetic construct
  source 1..374: mol_type = other RNA
ggccatcagtcgccggtcccaagcccggataaaatgggagggggcgggaaaccgcctaaccatgccgactgatggcagataatctgttggttataaatgacatttcctctggcaacttcaggtgcacgaaaccctggtgtgccctctgatccaagcatccccattctacagtaggactgagcactgccgagctgggcaatcttgagtcggatggcagcattgggatacagtgtgaaaagcagcacattgtggggtttcaggtatcggtatataatcatggcatatactgccatcagtcggcgtggactgtagaacactgccaatgccggtcccaagcccggataaaagtggagggtacagtccacgcttttttt

SEQ ID NO: 419 - RNA, 374 nt, synthetic construct
  source 1..374: mol_type = other RNA
ggccatcagtcgccggtcccaagcccggataaaatgggagggggcgggaaaccgcctaaccatgccgactgatggcagataatctgttggttataaatgacatttcctgaccgtacttcaggtgcacgattgggaggtgtgccctctgatccaagcatccccattctacagtaggactgagcactgccgagctgggcaatcttgagtcggatggcagcattgggtatgtctgtgaaaagcagcactaacacgggtttcaggtatcggtatataatcatggcatatactgccatcagtcggcgtggactgtagaacactgccaatgccggtcccaagcccggataaaagtggagggtacagtccacgcttttttt

SEQ ID NO: 420 - RNA, 372 nt, synthetic construct
  source 1..372: mol_type = other RNA
ggccatcagtcgccggtcccaagcccggataaaatgggagggggcgggaaaccgcctaaccatgccgactgatggcagataatggttataaatgacatttcctctggcaacttcaggtgcacgaaaccctggtgtgccctctgatgttgttatccggcgagtacagcagttcggaggaatcccgaggtcagcaatcctaagtatgatggcagcattgggatacagtgtgaaaagcagcacattgtggggtttcaggtatcggtatataatcatggctgaatatactgccatcagtcggcgtggactgtagaacactgccaatgccggtcccaagcccggataaaagtggagggtacagtccacgcttttttt

SEQ ID NO: 421 - RNA, 372 nt, synthetic construct
  source 1..372: mol_type = other RNA
ggccatcagtcgccggtcccaagcccggataaaatgggagggggcgggaaaccgcctaaccatgccgactgatggcagataatggttataaatgacatttcctctggcaacttgtccaccacgaaaccctggtgacgggactgatgttgttatccggcgagtacagcagttcggaggaatcccgaggtcagcaatcctaagtatgatggcagcattgcctatgagtgtgaaaagcagctgtaactggggtttcaggtatcggtatataatcatggctgaatatactgccatcagtcggcgtggactgtagaacactgccaatgccggtcccaagcccggataaaagtggagggtacagtccacgcttttttt

SEQ ID NO: 422 - RNA, 304 nt, synthetic construct
  source 1..304: mol_type = other RNA
ggccatcagtcgccggtcccaagcccggataaaatgggagggggcgggaaaccgcctaaccatgccgactgatggcagataatcaggtgcacgaaaccctggtgtgccctctgatccaagcatccccattctacagtaggactgagcactgccgagctgggcaatcttgagtcggatggcagcattgggatacagtgtgaaaagcagcacaatatactgccatcagtcggcgtggactgtagaacactgccaatgccggtcccaagcccggataaaagtggagggtacagtccacgcttttttt

SEQ ID NO: 423 - RNA, 334 nt, synthetic construct
  source 1..334: mol_type = other RNA
ggccatcagtcgccggtcccaagcccggataaaatgggagggggcgggaaaccgcctaaccatgccgactgatggcagataatttcctctggcaacttcaggtgcacgaaaccctggtgtgccctctgatccaagcatccccattctacagtaggactgagcactgccgagctgggcaatcttgagtcggatggcagcattgggatacagtgtgaaaagcagcacattgtggggtttcaggatatactgccatcagtcggcgtggactgtagaacactgccaatgccggtcccaagcccggataaaagtggagggtacagtccacgcttttttt

SEQ ID NO: 424 - RNA, 302 nt, synthetic construct
  source 1..302: mol_type = other RNA
ggccatcagtcgccggtcccaagcccggataaaatgggagggggcgggaaaccgcctaaccatgccgactgatggcagataatgcacgaaaccctggtgtgccctctgatgttgttatccggcgagtacagcagttcggaggaatcccgaggtcagcaatcctaagtatgatggcagcattgggatacagtgtgaaaagcagcacattgatatactgccatcagtcggcgtggactgtagaacactgccaatgccggtcccaagcccggataaaagtggagggtacagtccacgcttttttt

SEQ ID NO: 425 - RNA, 332 nt, synthetic construct
  source 1..332: mol_type = other RNA
ggccatcagtcgccggtcccaagcccggataaaatgggagggggcgggaaaccgcctaaccatgccgactgatggcagataatctggcaacttcaggtgcacgaaaccctggtgtgccctctgatgttgttatccggcgagtacagcagttcggaggaatcccgaggtcagcaatcctaagtatgatggcagcattgggatacagtgtgaaaagcagcacattgtggggtttcaggtctatatactgccatcagtcggcgtggactgtagaacactgccaatgccggtcccaagcccggataaaagtggagggtacagtccacgcttttttt

SEQ ID NO: 426 - RNA, 372 nt, synthetic construct
  source 1..372: mol_type = other RNA
ggccatcagtcgccggtcccaagcccggataaaatgggagggggcgggaaaccgcctaaccatgccgactgatggcagataatggttataaatgacatttcctctggcaacttcaggtgcacgaaaccctggtgacgggactgatgttgttatccggcgagtacagcagttcggaggaatcccgaggtcagcaatcctaagtatgatggcagcattgcctatgagtgtgaaaagcagcacattgtggggtttcaggtatcggtatataatcatggctgaatatactgccatcagtcggcgtggactgtagaacactgccaatgccggtcccaagcccggataaaagtggagggtacagtccacgcttttttt

SEQ ID NO: 427 - RNA, 364 nt, synthetic construct
  source 1..364: mol_type = other RNA
ggccatcagtcgccggtcccaagcccggataaaatgggagggggcgggaaaccgcctaaccatgccgactgatggcagataatggttataaatgacatttcctctggcaaccaggtgcacgaaaccctggtgacgggactgatgttgttatccggcgagtacagcagttcggaggaatcccgaggtcagcaatcctaagtatgatggcagcattgcctatgagtgtgaaaagcagcacattgtggggcaggatcggataaatcatggctgaatatactgccatcagtcggcgtggactgtagaacactgccaatgccggtcccaagcccggataaaagtggagggtacagtccacgcttttttt

SEQ ID NO: 428 - DNA, 20 bp, synthetic construct
  source 1..20: mol_type = other DNA
acaaagccagcctcactaga

SEQ ID NO: 429 - DNA, 20 bp, synthetic construct
  source 1..20: mol_type = other DNA
tcaaagacctgggcagaagt

SEQ ID NO: 430 - DNA, 21 bp, synthetic construct
  source 1..21: mol_type = other DNA
gtgtgccctctgatgtttttc

SEQ ID NO: 431 - DNA, 20 bp, synthetic construct
  source 1..20: mol_type = other DNA
cccaatgcttcgcttattgc

SEQ ID NO: 432 - DNA, 13 bp, synthetic construct
  source 1..13: mol_type = other DNA
  modified_base 1: mod_base = OTHER; note = 5' 6-Fluorescein-acaacagta-ZEN quencher attached to cytidine
  modified_base 13: mod_base = OTHER; note = guanosine-3' Iowa Black FQ
ctgagctatcccg

SEQ ID NO: 433 - DNA, 22 bp, synthetic construct
  source 1..22: mol_type = other DNA
tgtagttgaggtcaatgaaggg

SEQ ID NO: 434 - DNA, 19 bp, synthetic construct
  source 1..19: mol_type = other DNA
acatcgctcagacaccatg

SEQ ID NO: 435 - DNA, 16 bp, synthetic construct
  source 1..16: mol_type = other DNA
  modified_base 1: mod_base = OTHER; note = 5' hexachlorofluorescein-aaggtcgga-ZEN quencher attached to guanosine
  modified_base 16: mod_base = OTHER; note = cytidine-3' Iowa Black FQ
gtcaacggatttggtc

SEQ ID NO: 436 - RNA, 10 nt, synthetic construct
  source 1..10: mol_type = other RNA
ctacagcatt

SEQ ID NO: 437 - RNA, 10 nt, synthetic construct
  source 1..10: mol_type = other RNA
tatcccgaat

SEQ ID NO: 438 - RNA, 10 nt, synthetic construct
  source 1..10: mol_type = other RNA
tacagcattg

SEQ ID NO: 439 - RNA, 10 nt, synthetic construct
  source 1..10: mol_type = other RNA
aaatcccgat

SEQ ID NO: 440 - DNA, 26 bp, synthetic construct
  source 1..26: mol_type = other DNA
cagaaaacctgctccaaaaattccac

SEQ ID NO: 441 - DNA, 25 bp, synthetic construct
  source 1..25: mol_type = other DNA
  misc_difference 25: note = a, g, c, or t
tttttttttttttttttttttttvn